(19) United States
(12) Patent Application Publication
(10) Pub. No.: US 2024/0175006 A1
(43) Pub. Date: May 30, 2024

(54) COMPACT PROMOTERS FOR GENE EDITING

(71) Applicant: HUNTERIAN MEDICINE LLC, Cambridge, MA (US)

(72) Inventor: Vinod Jaskula-Ranga, Cambridge, MA (US)

(21) Appl. No.: 18/285,370

(22) PCT Filed: Mar. 31, 2022
(86) PCT No.: PCT/US22/22923

§ 371 Date: Oct. 2, 2023

Related U.S. Application Data
(60) Provisional application No. 63/168,769, filed on Mar. 31, 2021.

(51) Int. Cl. C12N 15/10; C12N 9/22; C12N 15/11; C12N 15/86

(52) U.S. Cl. CPC C12N 15/1024; C12N 9/22; C12N 15/111; C12N 15/86; C12N 2750/14143; C12N 2800/22; C12N 2830/15; C12N 2830/42

(57) ABSTRACT

The invention relates generally to compact promoters and their use in gene editing e.g., for treating disease. The disclosure is based, in part, upon the discovery of compact, bidirectional promoters that can be used to express both a nuclease (e.g., a Cas9 nuclease) and a guide RNA (gRNA). For example, in certain embodiments disclosed herein, a compact, bidirectional promoter can comprise at least one regulatory element that directs expression of a gRNA in one direction and at least one regulatory element that directs expression of a nuclease in the other direction. Accordingly, the promoters disclosed herein use less space than prior art promoters, allowing both a nuclease and a gRNA to be packaged in a single vector (e.g., a plasmid or an AAV).

### CROSS-REFERENCE TO RELATED APPLICATIONS

This application claims the benefit of and priority to U.S. Provisional Application No. 63/168,769, filed Mar. 31, 2021, the entire contents of which are incorporated by reference herein.

### FIELD OF THE INVENTION

The invention relates generally to compact promoters and their use in expressing gene editing systems, e.g., for treating disease.

### BACKGROUND

The development of CRISPR/Cas9 technology has revolutionized the field of gene editing. The CRISPR/Cas9 system is composed of a guide RNA (gRNA) that targets the Cas9 nuclease to sequence-specific DNA. Generating constructs for the CRISPR/Cas9 system is simple and fast, and targets can be multiplexed. Cleavage by the CRISPR system requires complementary base pairing of the gRNA to a 20-nucleotide DNA sequence and the requisite protospacer-adjacent motif (PAM), a short nucleotide motif found 3′ to the target site.

For in vivo gene targeting, the required CRISPR/Cas9 effector molecules are delivered to target cells by administration of appropriately engineered vectors, such as AAV vectors. For example, serotype 5 vector (AAV5) has been shown to be very efficient at transducing both nonhuman primate (Mancuso et al. (2009) NATURE 461, 784-787) and canine (Beltran et al. (2012) PROCEEDINGS OF THE NATIONAL ACADEMY OF SCIENCES OF THE UNITED STATES OF AMERICA 109, 2132-2137) photoreceptors and to be capable of mediating retinal therapy.

An important challenge in delivering Cas9 and guide RNAs via AAV is that the DNA required to express both components exceeds the packaging limit of AAV, approximately 4.7-4.9 kb, while the DNA required to express Cas9 and the gRNA, by conventional methods, exceeds 5 kb (promoter, ˜500 bp: spCas9, 4.140 bp: Pol II terminator, ˜250 bp: U6 promoter, ˜315 bp: and the gRNA, ˜100 bp). Swiech et al. (2015, NATURE BIOTECHNOLOGY 33, 102-106) addressed this challenge by using a two-vector approach: one AAV vector to deliver the Cas9 and another AAV vector for the delivery of gRNA. However, the double AAV approach in this study took advantage of a particularly small promoter, the murine Mecp2 promoter, which although expressed in retinal cells is not expressed in rods (Song et al. (2014) EPIGENETICS & CHROMATIN 7, 17: Jain et al. (2010) PEDIATRIC NEUROLOGY 43, 35-40). Thus this system as constructed would be suitable only for therapeutic interventions in certain areas of the retina, not including the rods.

Accordingly, there is a need in the art for constructs that allow for the production of gene editing systems including both a nuclease and gRNA that fit in a single vector, e.g., an AAV vector, and can drive expression in a variety of cell and tissue types.

### SUMMARY OF THE INVENTION

The disclosure is based, in part, upon the discovery of compact, bidirectional promoters that can be used to express both a nuclease (e.g., a Cas9 nuclease) and a guide RNA (gRNA). For example, in certain embodiments disclosed herein, a compact, bidirectional promoter can comprise at least one regulatory element that directs expression of a gRNA in one direction and at least one regulatory element that directs expression of a nuclease in the other direction. Accordingly, the promoters disclosed herein use less space than prior art promoters, allowing both a nuclease and a gRNA to be packaged in a single vector (e.g., a plasmid or an AAV).

In one aspect, the disclosure relates to a non-naturally occurring nuclease system including a vector including a compact bidirectional promoter, wherein the compact bidirectional promoter comprises: a) at least one regulatory element that provides for transcription in one direction of at least one nucleotide sequence encoding a guide RNA (gRNA), wherein the gRNA hybridizes with a target sequence of a nucleic acid: and b) at least one regulatory element that provides for transcription in the opposite direction of a nucleotide sequence encoding a nuclease, wherein the gRNA targets and hybridizes with the target sequence and directs the nuclease to the nucleic acid, wherein the bidirectional promoter is not one or more of SEQ ID NO: 70-SEQ ID NO: 106 and SEQ ID NO: 241-SEQ ID NO: 255).

In another aspect, the disclosure relates to a non-naturally occurring nuclease system including a vector including a compact bidirectional promoter, wherein the compact bidirectional promoter comprises both RNA pol II and RNA pol III activity, wherein a) the promoter provides for transcription of at least one nucleotide sequence encoding a guide RNA (gRNA), wherein the gRNA hybridizes with a target sequence of a nucleic acid: and b) the promoter provides for transcription of a nucleotide sequence encoding a nuclease, wherein the gRNA targets and hybridizes with the target sequence and directs the nuclease to the nucleic acid, wherein the bidirectional promoter is not one or more of SEQ ID NO: 70-SEQ ID NO: 106 and SEQ ID NO: 241-SEQ ID NO: 255.

In certain embodiments, the compact bidirectional promoter is between 50 and 225 bp. In certain embodiments, the compact bidirectional promoter is between 50 and 200 bp. In certain embodiments, the compact bidirectional promoter is between 50 and 180 bp.

In certain embodiments, the compact bidirectional promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

In certain embodiments, the compact bidirectional promoter comprises an H1 promoter. In certain embodiments, the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

In certain embodiments, the compact bidirectional promoter comprises a Gar1 promoter. In certain embodiments, the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto. In certain embodiments, the Gar1 promoter is a human Gar1 promoter.

In certain embodiments, the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

In certain embodiments, the compact promoter comprises at least 95%, 98%, 99%, 99.5% or 100% identity to a naturally-occurring mammalian promoter.

In certain embodiments, the target sequence comprises the nucleotide sequence

AN₁₉NGG,
GN₁₉NGG,
CN₁₉NGG,
or
TN₁₉NGG.

In certain embodiments, the nuclease is an RNA-directed nuclease. In certain embodiments, the RNA-directed nuclease is a Cas protein. In certain embodiments, the Cas protein is codon optimized for expression in the cell and/or is a Type-II Cas protein or a Type-V Cas protein. In certain embodiments, the cell is a eukaryotic cell. In certain embodiments, the eukaryotic cell is a mammalian cell. In certain embodiments, the eukaryotic cell is a human cell.

In certain embodiments, the system is packaged into a single vector.

In another aspect, the disclosure relates to an expression construct including a nuclease system as described herein.

In another aspect, the disclosure relates to a vector including an expression construct as described herein. In certain embodiments, the vector comprises an adeno-associated viral (AAV) vector. In certain embodiments, the AAV vector comprises an AAV-6 vector.

In another aspect, the disclosure relates to a method that includes introducing into a cell a non-naturally occurring nuclease system including a vector including a compact bidirectional promoter, wherein the compact bidirectional promoter comprises: a) at least one regulatory element that provides for transcription in one direction of at least one nucleotide sequence encoding a guide RNA (gRNA), wherein the gRNA hybridizes with a target sequence of a nucleic acid molecule: and b) at least one regulatory element that provides for transcription in the opposite direction of a nucleotide sequence encoding a nuclease, wherein the gRNA targets and hybridizes with the target sequence and directs the nuclease to the nucleic acid molecule, wherein the bidirectional promoter is not one or more of SEQ ID NO: 70-SEQ ID NO: 106 and SEQ ID NO: 241-SEQ ID NO: 255.

In another aspect, the disclosure relates to a method including introducing into a cell a non-naturally occurring nuclease system including a vector including a compact bidirectional promoter, wherein the compact bidirectional promoter comprises both RNA pol II and RNA pol III activity, wherein a) the promoter provides for transcription of at least one nucleotide sequence encoding a guide RNA (gRNA), wherein the gRNA hybridizes with a target sequence of a nucleic acid: and b) the promoter provides for transcription of a nucleotide sequence encoding a nuclease, wherein the gRNA targets and hybridizes with the target sequence and directs the nuclease to the nucleic acid, wherein the bidirectional promoter is not one or more of SEQ ID NO: 70-SEQ ID NO: 106 and SEQ ID NO: 241-SEQ ID NO: 255.

In certain embodiments, the compact bidirectional promoter is between 50 and 225 bp. In certain embodiments, the compact bidirectional promoter is between 50 and 200 bp. In certain embodiments, the compact bidirectional promoter is between 50 and 180 bp.

In certain embodiments, the bidirectional promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

In certain embodiments, the compact bidirectional promoter comprises an H1 promoter. In certain embodiments, the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

In certain embodiments, the compact bidirectional promoter comprises a Gar1 promoter. In certain embodiments, the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto. In certain embodiments, the Gar1 promoter is a human Gar1 promoter.

In certain embodiments, the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

In certain embodiments, the compact promoter does not comprise a viral promoter and/or a synthetic promoter.

In certain embodiments, the compact promoter comprises at least 95%, 98%, 99%, 99.5% or 100% identity to a naturally-occurring mammalian promoter.

In certain embodiments, the target sequence comprises the nucleotide sequence

AN₁₉NGG,
GN₁₉NGG,
CN₁₉NGG,
or
TN₁₉NGG.

In certain embodiments, the nuclease is an RNA-directed nuclease. In certain embodiments, the RNA-directed nuclease is a Cas9 protein. In certain embodiments, the Cas9 protein is codon optimized for expression in the cell and/or is a Type-II Cas9 protein.

In certain embodiments, the cell is a eukaryotic cell optionally selected from the group consisting of (i) a mammalian cell, (ii) a human cell, and/or (iii) a retinal photoreceptor cell.

In certain embodiments, the system is packaged into a single adeno-associated virus (AAV) particle.

These and other aspects and features of the invention are described in the following detailed description and claims.

### DESCRIPTION OF THE DRAWINGS

The invention can be more completely understood with reference to the following drawings.

FIG. 1 is a schematic showing the region in which the H1 promoter is located, between the start of the H1RNA gene (left) to the start of the PARP-2 gene (right). Transcription factor binding sites including Staf, DSE, PSE, c-REL, GATA-1, GATA-2, and CREB are shown. In addition, the B recognition sequence (BRE) and TATA box are shown.

FIG. 2 provides Hidden Markov model (HMM) used to identify H1 promoter sequences.

FIG. 3 provides an alignment of Artiodactyla, Carnivora, Cetacea, Chiroptera, Insectivore, Lagomorpha, Marsupial, Pangolin, Perissodactyla, Primate, Rodent, and Xenartha H1 promoters.

FIG. 4 provides an alignment of human and Orycteropus afer H1 promoters, showing the 132 bp insertion and 12 bp insertion found in the Orycteropus afer H1 promoter. The human H1 promoter corresponds to SEQ ID NO: 87 and the Orycteropus afer H1 promoter corresponds to SEQ ID NO: 25. The consensus sequence corresponds to SEQ ID NO: 1808.

FIG. 5 provides an alignment of H1 promoter sequences from Artiodactyla species.

FIG. 6 provides an alignment of H1 promoter sequences from Carnivora species.

FIG. 7 provides an alignment of H1 promoter sequences from Cetacea species.

FIG. 8 provides an alignment of H1 promoter sequences from Chiroptera species.

FIG. 9 provides an alignment of H1 promoter sequences from Dermoptera species.

FIG. 10 provides an alignment of H1 promoter sequences from Hyracoidae species.

FIG. 11 provides an alignment of H1 promoter sequences from Insectivora species.

FIG. 12 provides an alignment of H1 promoter sequences from Lagomorpha species.

FIG. 13 provides an alignment of H1 promoter sequences from Marsupial species.

FIG. 14 provides an alignment of H1 promoter sequences from Pangolin species.

FIG. 15 provides an alignment of H1 promoter sequences from Perissodactyla species.

FIG. 16 provides an alignment of H1 promoter sequences from Primate species.

FIG. 17 provides an second alignment of H1 promoter sequences from Primate species showing the TATA box, PSE, Staf, and DSE binding sites.

FIG. 18 provides an alignment of H1 promoter sequences from Rodent species.

FIG. 19 provides an alignment of H1 promoter sequences from Xenartha species.

FIG. 20A depicts DNA alignment and conservation of the H1 bidirectional promoter, from the start of the H1RNA gene (left) to the start of the PARP-2 gene (right). FIG. 20B depicts RNA polymerase II-driven promoter activity in Hela cells. Also depicted is the length of each promoter shown in the red bars, plotted against the right Y axis.

FIG. 21 provides a schematic representation of mouse H1 promoter deletion constructs evaluated as described in Example 2.

FIG. 22 shows an alignment of mouse H1 promoter deletion constructs evaluated as described in Example 2.

FIG. 23 shows a bar graph showing normalized firefly to nanoluc luciferase signal for each mouse H1 promoter deletion constructs described in Example 2.

FIG. 24 provides a schematic representation of 17 mouse H1 promoter mutation constructs that were designed by walking across the promoter in 10 bp increments and replacing the sequence with its reverse complement.

FIG. 25 provides a sequence alignment of the mouse H1 promoter mutation constructs provided in FIG. 24.

FIG. 26 shows a bar graph showing normalized firefly to nanoluc luciferase signal for each mouse H1 promoter mutation constructs described in Example 3.

FIG. 27 provides a schematic representation of 12 constructs designed to incorporate introns into the mouse H1 promoter region.

FIG. 28 shows a bar graph showing normalized firefly to nanoluc luciferase signal for each mouse H1 intron constructs described in Example 4.

FIG. 29 provides a schematic showing the design of human H1 promoter and variant constructs. As shown in FIG. 29, a construct carrying a human H1 promoter alone (p144), a human H1 promoter with a 9 bp Kozak sequence (GCCGCCACC) (SEQ ID NO: 256) (p145), a human H1 promoter with a beta-globin 5′UTR (p146), and a human H1 promoter with a TATA box mutation (TATAA->TCGAA) (p147) were designed.

FIG. 30 provides a sequence alignment of the constructs provided in FIG. 29.

FIG. 31 shows a bar graph showing normalized firefly to nanoluc luciferase signal for each human H1 wt and 5′UTR construct described in Example 5.

FIG. 32 provides a schematic showing the design of mouse H1 promoter and 5′UTR variant constructs.

FIG. 33 provides a sequence alignment of the constructs provided in FIG. 32.

FIG. 34 shows a bar graph showing normalized firefly to nanoluc luciferase signal for each mouse H1 wt and 5′UTR construct described in Example 5.

FIG. 35 shows a bar graph showing normalized firefly to nanoluc luciferase signal for each bidirectional promoter construct described in Example 6. The promoters were human H1 (p144: SEQ ID NO: 87), mouse H1 (p148: SEQ ID NO: 93), human 7sk-1 (p199: SEQ ID NO: 242), mouse 7sk-1 (p203: SEQ ID NO: 204), human ALOXE3 (p204: SEQ ID NO: 246), human CGB1 (p206: SEQ ID NO: 247), human CGB2 (p207: SEQ ID NO: 248), human GAR1-1 (p216; SEQ ID NO: 107), human Med16-1 (p222: SEQ ID NO: 249), human Med16-2 (p223: SEQ ID NO: 250), human SRP (p242: SEQ ID NO: 233).

FIG. 36 is a graph showing the optimization of a luciferase reporter assay. HEK293 cells were co-transfected with firefly luciferase and NANOLUCR® reporter plasmids under the control of standard promoters p006 (EF1a), p323 (PGK), and p322 (TK). Normalized luciferase expression (firefly:NANOLUCR) was quantified for transfection ratios of 90:10 ng, 99: 1 ng, and 100:0.1 ng.

FIG. 37 is a bar graph showing normalized luciferase signal (firefly: NANOLUCR) for a library of H1 promoters including p095, p127, p110, p109, p088, p094, p060, p071, p077, p103, p100, p102, p092, p073, p100, p102, p092, p073, p083, p130, p066, p089, p112, p101, p099, p116, p098, p069, p106, p131, p081, p107, p074, p072, p082, p097, p108, p065, p122, p114, p070, p091, p062, p119, p113, p063, p064, p090, p079, p105, p067, p128, p124, p084, p126, p078, p086, p093, p059, p058, p087, p061, p085, p129, p096, p111, p125, p115, p068, p118, p117, p076, p120, p123, and p104 in CFBE410-cells. Control TK promoter normalized luciferase activity is shown as p322.

FIG. 38 is a bar graph showing normalized luciferase signal (firefly: NANOLUCR) for a library of H1 promoters including p095, p127, p088, p094, p087, p1 10, p109, p083, p100, p073, p116, p092, p077, p066, p130, p101, p079, p071, p081, p119, p065, p098, p097, p060, p061, p089, p078, p070, p102, p084, p086, p059, p099, p106, p069, p125, p117, p058, p067, p129, p126, p107, p122, p064, p112, p062, p085, p091, p082, p072, p131, p090, p093, p063, p068, p114, p120, p115, p074, p076, p108, p113, p096, p124, p105, p103, p118, p128, p111, p123, and p104 in A549 cells. Control TK promoter normalized luciferase activity is shown as p322.

FIG. 39 is a bar graph showing normalized luciferase signal (firefly: NANOLUCR) for a library of H1 promoters including p095, p127, p094, p110, p107, p109, p102, p084, p071, p087, p101, p088, p097, p092, p066, p077, p106, p065, p099, p078, p116, p081, p119, p083, p098, p131, p073, p112, p100, p062, p103, p091, p061, p072, p129, p068, p114, p120, p060, p070, p118, p059, p113, p089, p108, p069, p067, p122, p124, p058, p079, p115, p093, p130, p086, p074, p125, p063, p126, p117, p090, p076, p096, p128, p105, p111, p123, p085, p082, p064, and p104 in Calu3 cells. Control TK promoter normalized luciferase activity is shown as p322.

FIG. 40A is a violin plot showing log-scale expression of a library of H1 promoters in three lung cell types (CFBE410-, A549, and Calu3). Vertical axis represents relative luminescence units.

FIG. 40B is a violin plot showing log-scale expression of a library of H1 promoters in Calu-3 cells compared to the expression activity of standard promoters TK, PGK, and EF1a.

FIG. 41 is a series of graphs showing linear regression analysis to compare the expression activity of each of the promoters in the library (each dot on represents a promoter) in different cell types.

FIG. 42 is a plot showing hierarchical clustering of a library of H1 promoters segregated by activity in three lung cell types (CFBE410-marked with a*, A549 marked with a †, and Calu3 marked with a ‡ and one control cell type (HeLa marked with a ♦)

### DETAILED DESCRIPTION

Various features and aspects of the invention are discussed in more detail below.

The disclosure is based, in part, upon the discovery of compact, bidirectional promoters that can be used to express both a nuclease (e.g., a Cas9 nuclease) and a guide RNA (gRNA). For example, in certain embodiments disclosed herein, a compact, bidirectional promoter can comprise at least one regulatory element that directs expression of a gRNA in one direction and at least one regulatory element that directs expression of a nuclease in the other direction.

Accordingly, the disclosure provides nucleic acids, expression constructs, and vectors comprising a compact bidirectional promoter and a gene editing system, wherein the compact promoter is small enough to allow for the inclusion of both a nuclease and a guide RNA (gRNA) in a single vector, such as an AAV vector, which has a size limit that makes expression of both nuclease and gRNA difficult using conventional promoters.

Unless otherwise defined herein, scientific and technical terms used in this application shall have the meanings that are commonly understood by those of ordinary skill in the art.

Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), 0) microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press: Cell Biology: A Laboratory Notebook (J. E. Cellis, ed., 1998) Academic Press: Animal Cell Culture (R. I. Freshney, ed., 1987): Introduction to Cell and Tissue Culture (J. P. Mather and P. E. Roberts, 1998) Plenum Press: Cell and Tissue Culture: Laboratory Procedures (A. Doyle, J. B. Griffiths, and D. G. Newell, eds., 1993-1998) J. Wiley and Sons: Methods in Enzymology (Academic Press, Inc.): Gene Transfer Vectors for Mammalian Cells (J. M. Miller and M. P. Calos, eds., 1987): Current Protocols in Molecular Biology (F. M. Ausubel et al., eds., 1987): PCR: The Polymerase Chain Reaction, (Mullis et al., eds., 1994): Sambrook and Russell, Molecular Cloning: A Laboratory Manual, 3rd. ed., Cold Spring Harbor Laboratory Press, Cold Spring Harbor, NY (2001): Ausubel et al., Current Protocols in Molecular Biology, John Wiley & Sons, NY (2002): Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003): Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Throughout this specification and embodiments, the word “comprise,” or variations such as “comprises” or “comprising.” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10: that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in an embodiment of the disclosure.

Exemplary methods and materials are described herein, although methods and materials similar or equivalent to those described herein can also be used in the practice or testing of the present disclosure. The materials, methods, and examples are illustrative only and not intended to be limiting.

### I. Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5 ‘ and 3’ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

IUPAC nucleotide code is used throughout. IUPAC nucleotide code is provided in TABLE 1.

TABLE 1

| | |
|---|---|
| A | Adenine |
| C | Cytosine |
| G | Guanine |
| T (or U) | Thymine (or Uracil) |
| R | A or G |
| Y | C or T |
| S | G or C |
| W | A or T |
| K | G or T |
| M | A or C |
| B | C or G or T |
| D | A or G or T |
| H | A or C or T |
| V | A or C or G |
| N | any base |
| . or - | gap |

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention: for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As used herein, the term “functional fragment” refers to a fragment of (a) a promoter or (b) a gene or coding sequence (e.g., an mRNA) that encodes a protein (e.g., a nuclease) that retains, for example, at least 10%, at least 20%, at least 30%, at least 40%, at least 50%, at least 60%, at least 70%, at least 80%, at least 90%, at least 95%, or 100% of at least one activity of the corresponding full-length, naturally occurring promoter or protein.

As used herein, the term “variant” refers to a variant of (a) a promoter or (b) a gene or coding sequence (e.g., an mRNA) that encodes a protein (e.g., a nuclease) that retains, for example, at least 10%, at least 20%, at least 30%, at least 40%, at least 50%, at least 60%, at least 70%, at least 80%, at least 90%, at least 95%, or 100% of at least one activity of the corresponding full-length, naturally occurring promoter or protein. For example, a variant can comprise a splice variant or a gene comprising a mutation such as an insertion, deletion, or substitution.

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions.

However, in common usage and in the instant application, the term “homologous,” when modified with an adverb such as “highly,” may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

Within a recombinant expression vector, “operably linked” is intended to mean that the nucleotide sequence of interest is linked to the regulatory element(s) in a manner that allows for expression of the nucleotide sequence: (e.g., in an in vitro transcription/translation system or in a host cell when the vector is introduced into the host cell).

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel (1990) Gene Expression Technology: Methods in Enzymology 185, Academic Press, San Diego Calif. Regulatory elements include those that direct constitutive expression. Of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may not also be tissue or cell-type specific.

In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to the retroviral Rous sarcoma virus (RSV) LTR promoter (optionally with the RSV enhancer), the cytomegalovirus (CMV) promoter (optionally with the CMV enhancer) (e.g., Boshart et al. (1985) Cell 41:521-530), the SV40 promoter, the dihydrofolate reductase promoter, the B-actin promoter, the phosphoglycerol kinase (PGK) promoter, and the EF1a promoter.

Also encompassed by the term “regulatory element” are enhancer elements, such as WPRE: CMV enhancers: the R-US' segment in LTR of HTLV-I (Takebe et al. (1988) MOL. CELL. BIOL. 8:466-472): SV40 enhancer: and the intron sequence between exons 2 and 3 of rabbit.beta.- globin (O'Hare et al. (1981) PROC. NATL. ACAD. SCI. USA. 78(3):1527-31). It will be appreciated by those skilled in the art that the design of the expression vector can depend on such factors as the choice of the host cell to be transformed, the level of expression desired, etc.

A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.). Advantageous vectors include lentiviruses and adeno-associated viruses, and types of such vectors can also be selected for targeting particular types of cells.

In aspects of the presently disclosed subject matter the terms “chimeric RNA,” “chimeric guide RNA,” “guide RNA,” “single guide RNA” and “synthetic guide RNA” are used interchangeably and refer to the polynucleotide sequence comprising the guide sequence. The term “guide sequence” refers to the about 20 bp sequence within the guide RNA that specifies the target site and may be used interchangeably with the terms “guide” or “spacer”.

As used herein the term “wild type” is a term of the art understood by skilled persons and means the typical form of an organism, strain, gene or characteristic as it occurs in nature as distinguished from mutant or variant forms.

The terms “non-naturally occurring” and “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, a “host cell” includes an individual cell or cell culture that can be or has been a recipient for vector(s) for incorporation of polynucleotide inserts. The term host cell may refer to the packaging cell line in which the rAAV is produced from the plasmid. In the alternative, the term “host cell” may refer to the target cell in which expression of the transgene is desired.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e. a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

A “recombinant AAV vector (rAAV vector)” refers to a polynucleotide vector based on an adeno-associated virus comprising one or more heterologous sequences (i.e., nucleic acid sequence not of AAV origin) that are flanked by at least one AAV inverted terminal repeat sequence (ITR). Such rAAV vectors can be replicated and packaged into infectious viral particles when present in a host cell that has been infected with a suitable helper virus (or that is expressing suitable helper functions) and that is expressing AAV rep and cap gene products (i.e. AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. An rAAV vector can be in any of a number of forms, including, but not limited to, plasmids, linear artificial chromosomes, complexed with lipids, encapsulated within liposomes, and encapsidated in a viral particle, e.g., an AAV particle. An rAAV vector can be packaged into an AAV virus capsid to generate a “recombinant adeno-associated viral particle (rAAV particle)”.

An “TAAV virus” or “rAAV viral particle” refers to a viral particle composed of at least one AAV capsid protein and an encapsidated rAAV vector genome.

The term “transgene” refers to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

An “inverted terminal repeat” or “ITR” sequence is a term well understood in the art and refers to relatively short sequences found at the termini of viral genomes which are in opposite orientation.

An “AAV inverted terminal repeat (ITR)” sequence, a term well-understood in the art, is an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C and D regions), allowing intrastrand base-pairing to occur within this portion of the ITR. A “helper virus” for AAV refers to a virus that allows AAV (which is a defective parvovirus) to be replicated and packaged by a host cell. A number of such helper viruses are known in the art.

As used herein, “expression control sequence” means a nucleic acid sequence that directs transcription of a nucleic acid. An expression control sequence can be a promoter, such as a constitutive promoter, or an enhancer. The expression control sequence is operably linked to the nucleic acid sequence to be transcribed.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity (ies) in the composition).

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure.

The terms “patient,” “subject,” or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some embodiments, the subject is a human that is at least 40, 45, 50, 55, 60, 65, 70, 75, 80, 85, 90 or 95 years of age.

As used herein, the terms “prevent,” “preventing” and “prevention” refer to the prevention of the recurrence or onset of, or a reduction in one or more symptoms of a disease or condition in a subject as result of the administration of a therapy (e.g., a prophylactic or therapeutic agent). For example, in the context of the administration of a therapy to a subject for an infection, “prevent,” “preventing” and “prevention” refer to the inhibition or a reduction in the development or onset of a disease or condition, or the prevention of the recurrence, onset, or development of one or more symptoms of a disease or condition, in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition, treatment refers to the reduction or amelioration of the progression, severity, and/or duration of one or more symptoms of the disease, or the amelioration of one or more symptoms resulting from the administration of one or more therapies (including, but not limited to, the administration of one or more prophylactic or therapeutic agents).

“Administering” or “administration of a substance, a compound or an agent to a subject can be carried out using one of a variety of methods known to those skilled in the art. In some embodiments, administration may be local. In other embodiments, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Each embodiment described herein may be used individually or in combination with any other embodiment described herein.

### II. Compact Promoters

The disclosure is based, in part, upon the discovery that compact promoters can effectively drive expression of nuclease systems, for example, those including both a nuclease and a guide RNA (gRNA). The size limitations of AAV and other vectors (e.g., plasmids) make it difficult to package both a gRNA and a nuclease into a single vector. However, this problem can be overcome by using a compact promoter, as described herein, to deliver sufficient expression of a nuclease system via a single vector.

A compact promoter provided herein can be selected to express the selected nuclease system in a desired target cell. In some embodiments, the target cell is a retinal cell, lung cell, a pancreatic cell, a liver cell, or a neuronal cell. The promoter may be derived from any species, including human. In one embodiment, the promoter is “cell specific”. The term “cell-specific” means that the particular promoter selected for the recombinant vector can direct expression of the selected transgene in a particular cell.

In certain embodiments, the promoter is of a small size, e.g., less than about 500 bp, due to the size limitations of the AAV vector. In certain embodiments, the promoter is less than about 300 bp, less than about 200 bp, between about 50 bp and about 400 bp, between about 75 bp and about 400 bp, between about 99 bp and about 400 bp, between about 100 bp and about 400 bp, between about 150 bp and about 400 bp, between about between about 200 bp and about 400 bp, between about 250 bp and about 400 bp, between about 300 bp and about 400 bp, about 50 bp and about 300 bp, about 75 bp and about 300 bp, about 100 bp and about 300 bp, about 150 bp and about 300 bp, between about 200 bp and about 300 bp, about 50 bp and about 250 bp, about 75 bp and about 250 bp, between about 100 bp and about 250 bp, between about 150 bp and about 250 bp, between about 200 bp and about 250 bp, between about 50 bp and about 200 bp, between about 75 bp and about 200 bp, between about 100 bp and about 200 bp, between about 150 bp and about 200 bp, between about 50 bp and about 150 bp, between about 100 bp and about 150 bp, between about 50 bp and about 150 bp, and between about 100 bp and about 150 bp in size.

In certain embodiments, the promoter is a bidirectional promoter. In certain embodiments, the bidirectional promoter is less than about 500 bp. In certain embodiments, the bidirectional promoter is less than about 300 bp, less than about 200 bp, between about 50 bp and about 400 bp, between about 75 bp and about 400 bp, between about 99 bp and about 400 bp, between about 100 bp and about 400 bp, between about 150 bp and about 400 bp, between about between about 200 bp and about 400 bp, between about 250 bp and about 400 bp, between about 300 bp and about 400 bp, between about 50 bp and about 300 bp, between about 75 bp and about 300 bp, between about 100 bp and about 300 bp, between about 150 bp and about 300 bp, between about 200 bp and about 300 bp, between about 50 bp and about 250 bp, between about 75 bp and about 250 bp, between about 100 bp and about 250 bp, between about 150 bp and about 250 bp, between about 200 bp and about 250 bp, between about 50 bp and about 200 bp, between about 75 bp and about 200 bp, between about 100 bp and about 200 bp, between about 150 bp and about 200 bp, between about 50 bp and about 150 bp, between about 100 bp and about 150 bp, between about 50 bp and about 150 bp, and between about 100 bp and about 150 bp in size.

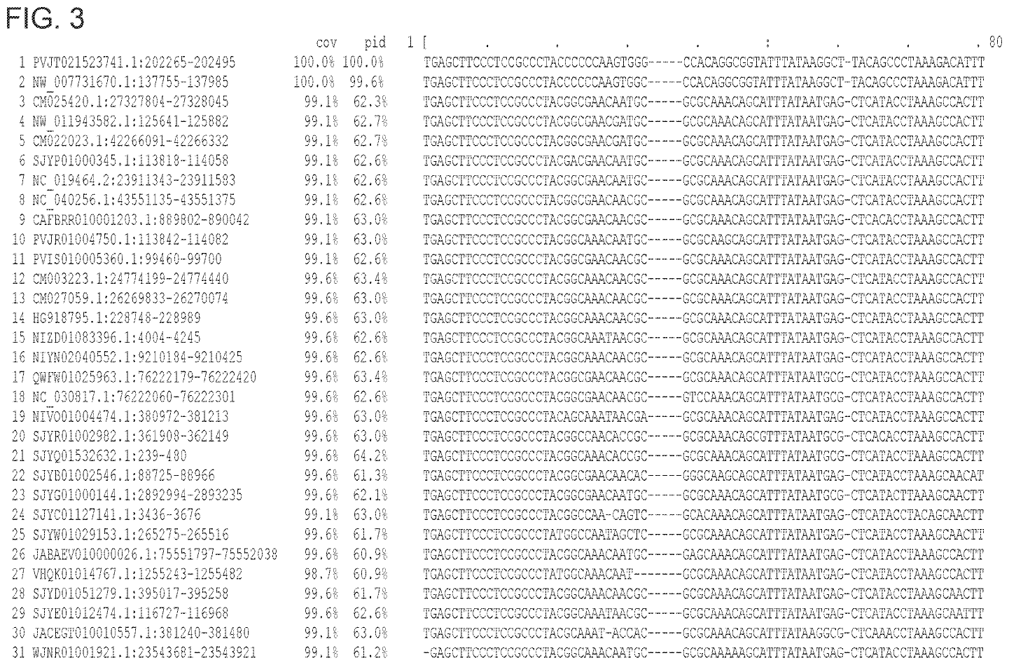

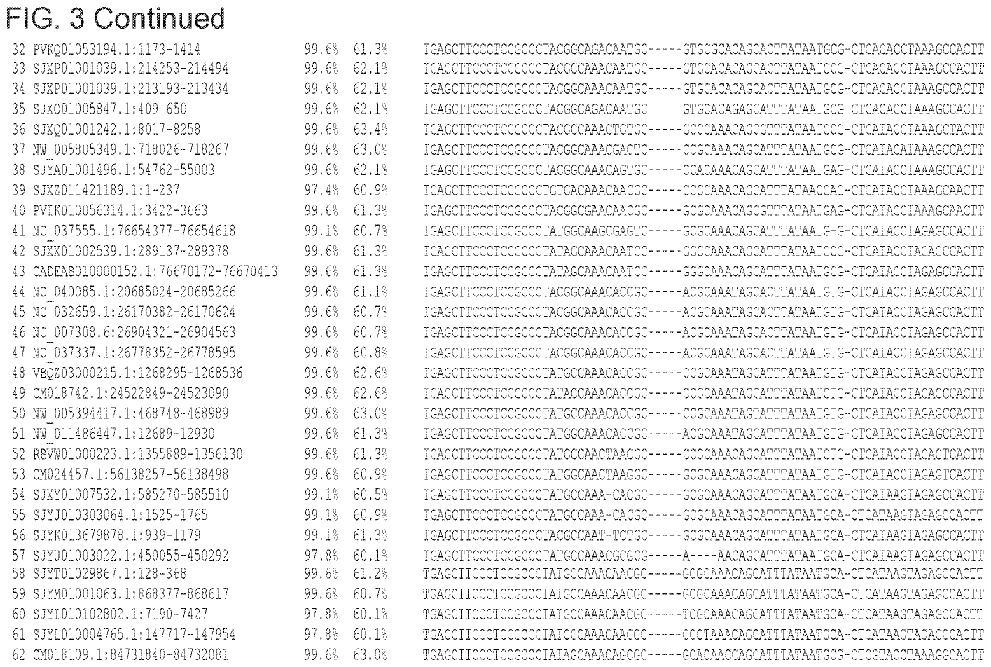

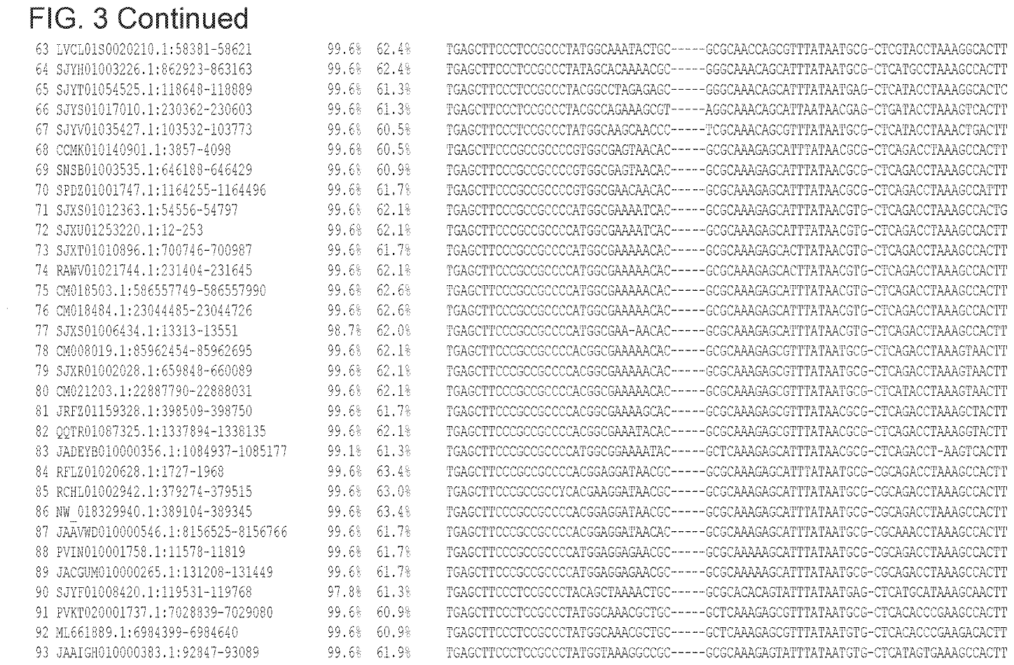

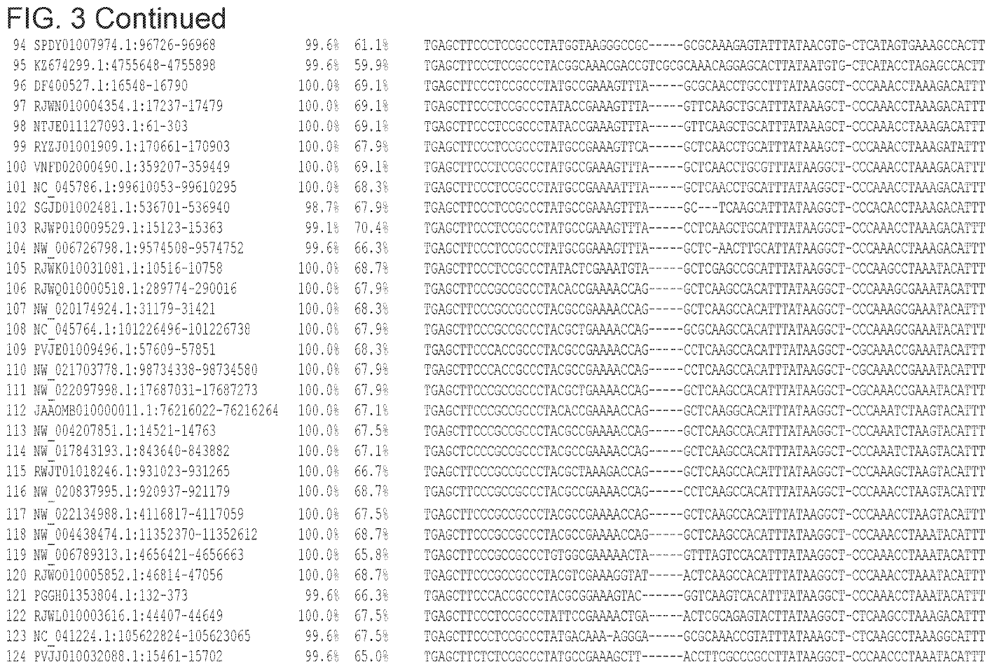

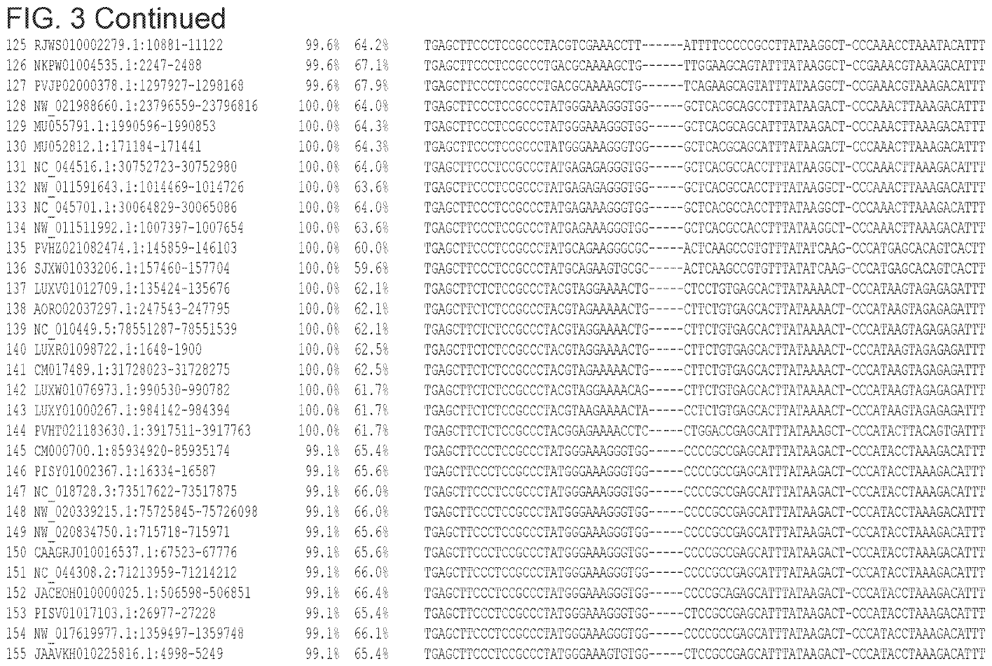

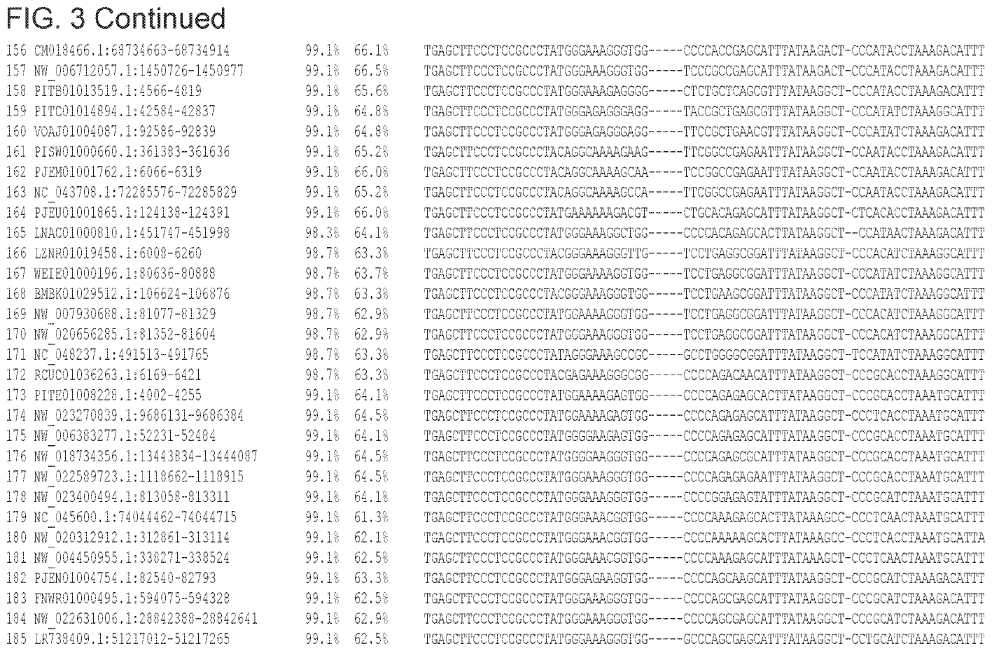

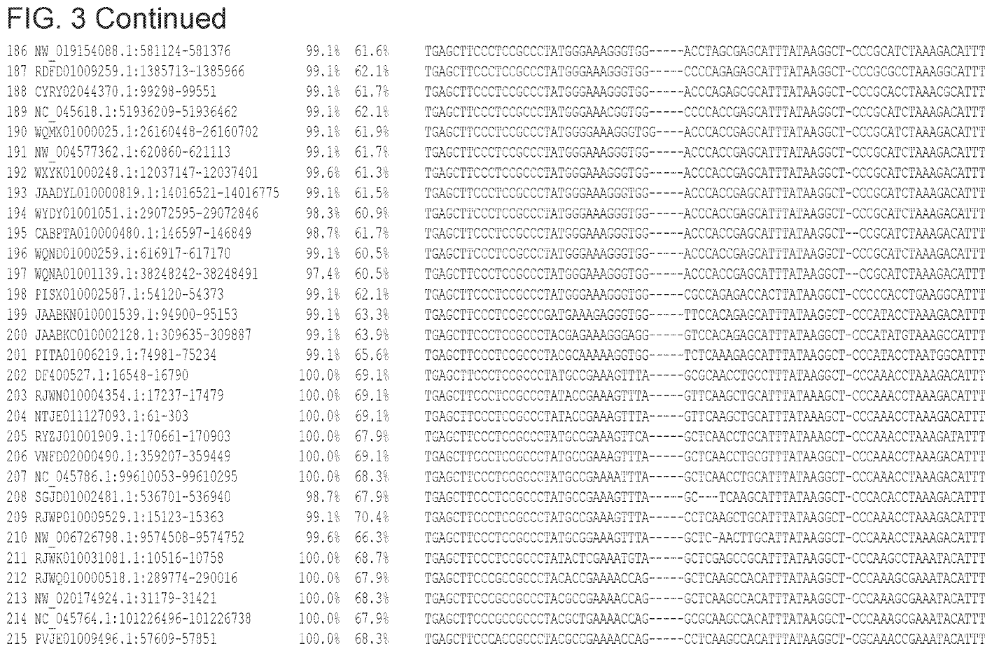

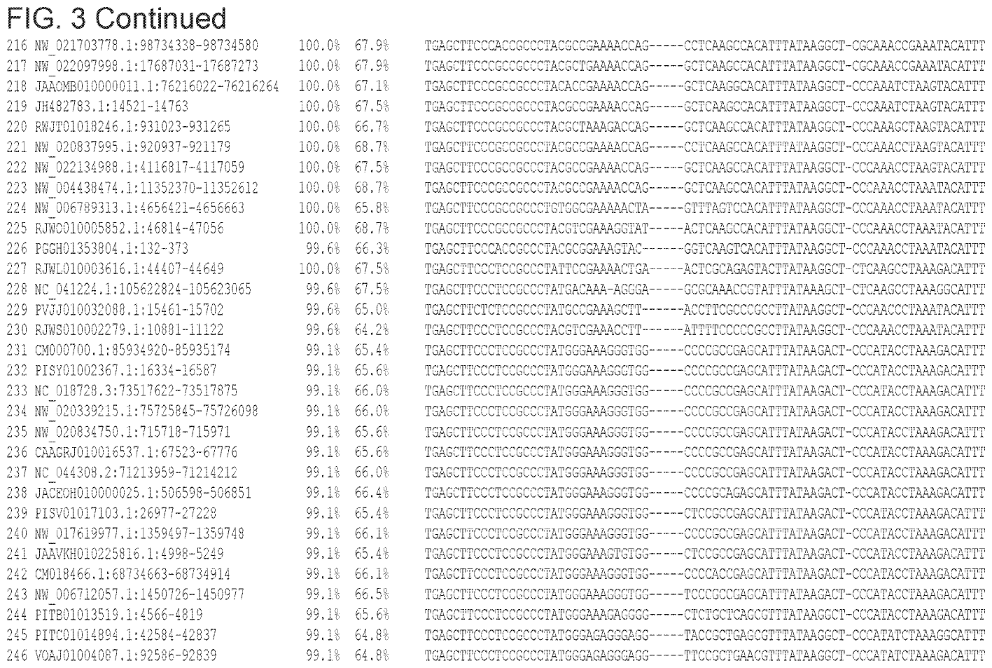

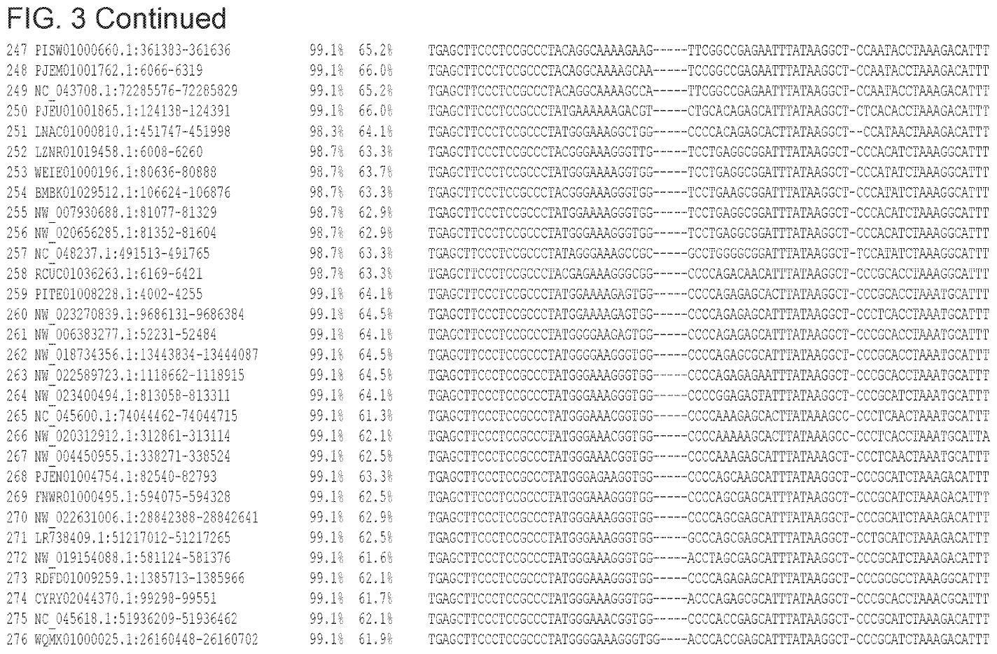

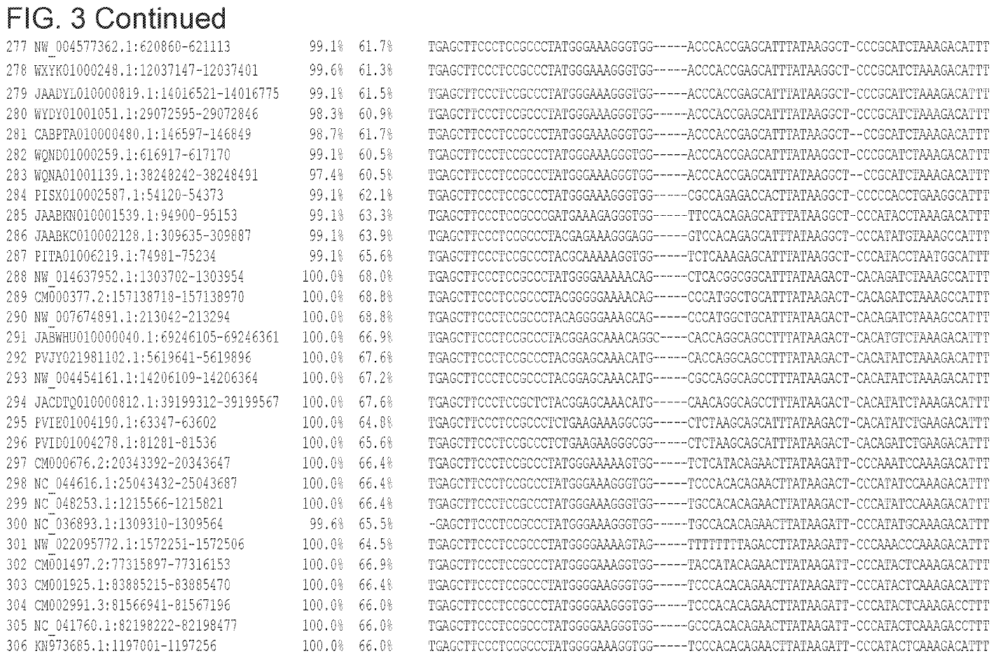

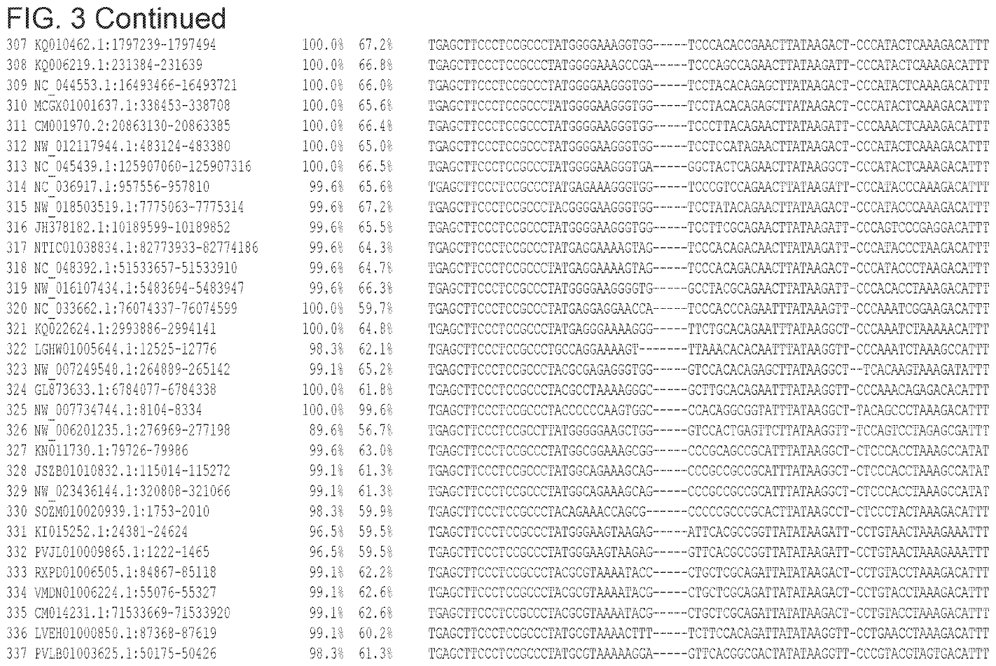

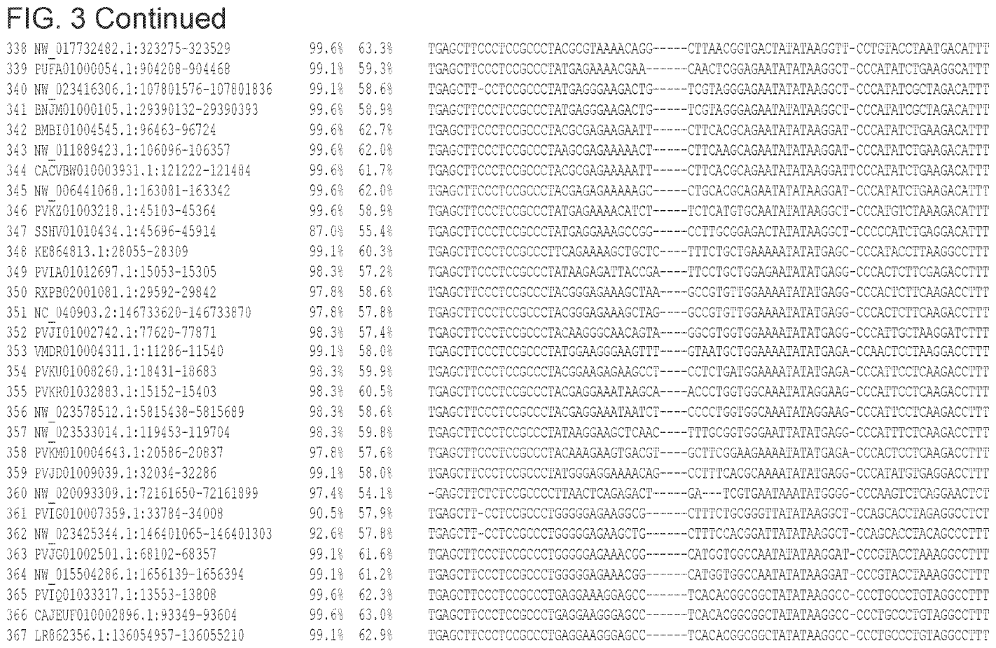

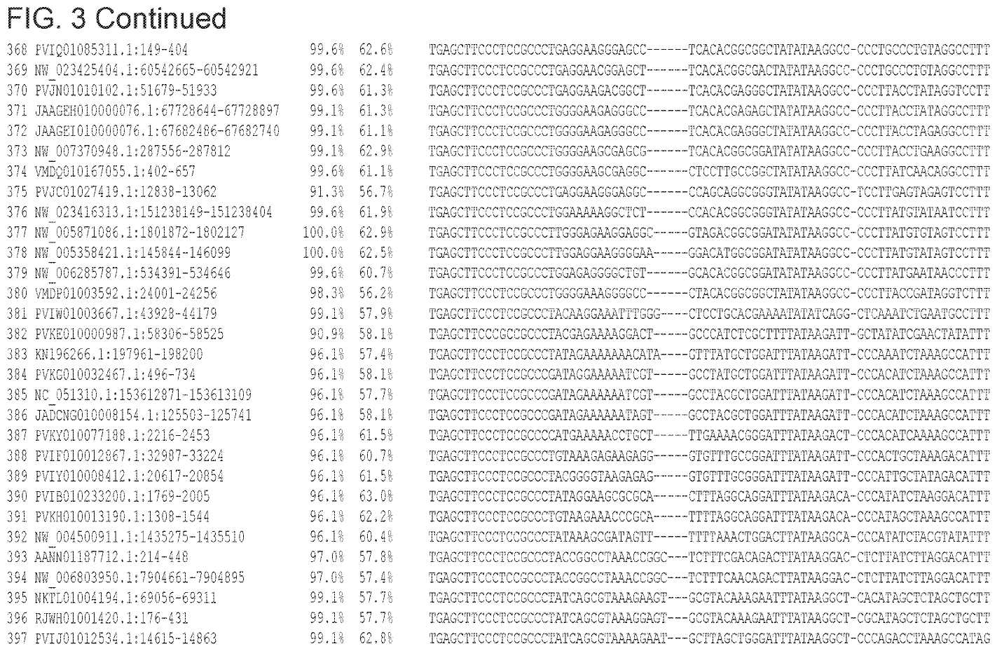

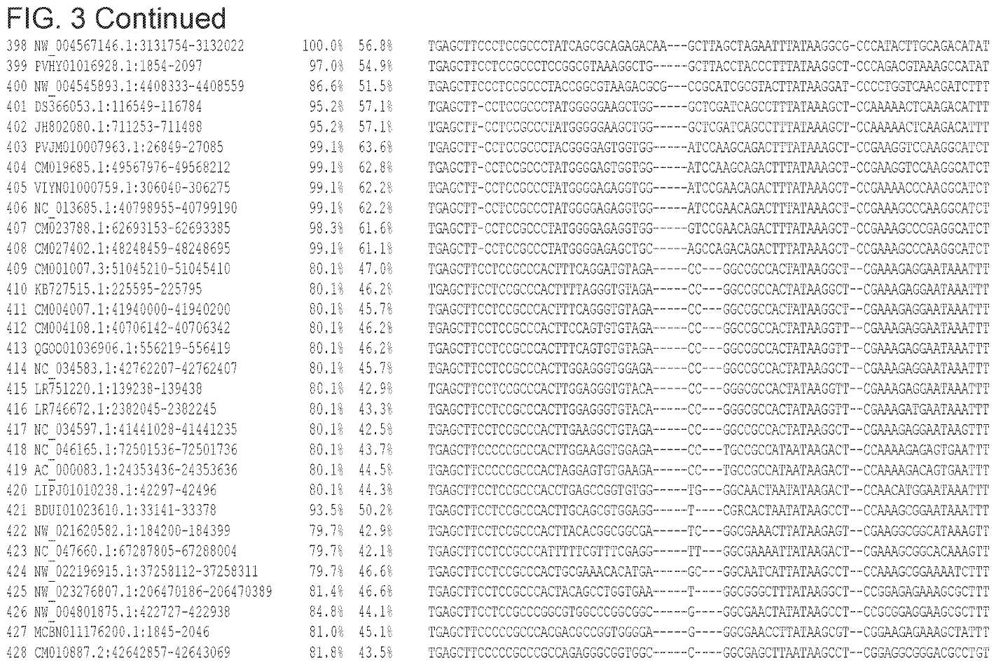

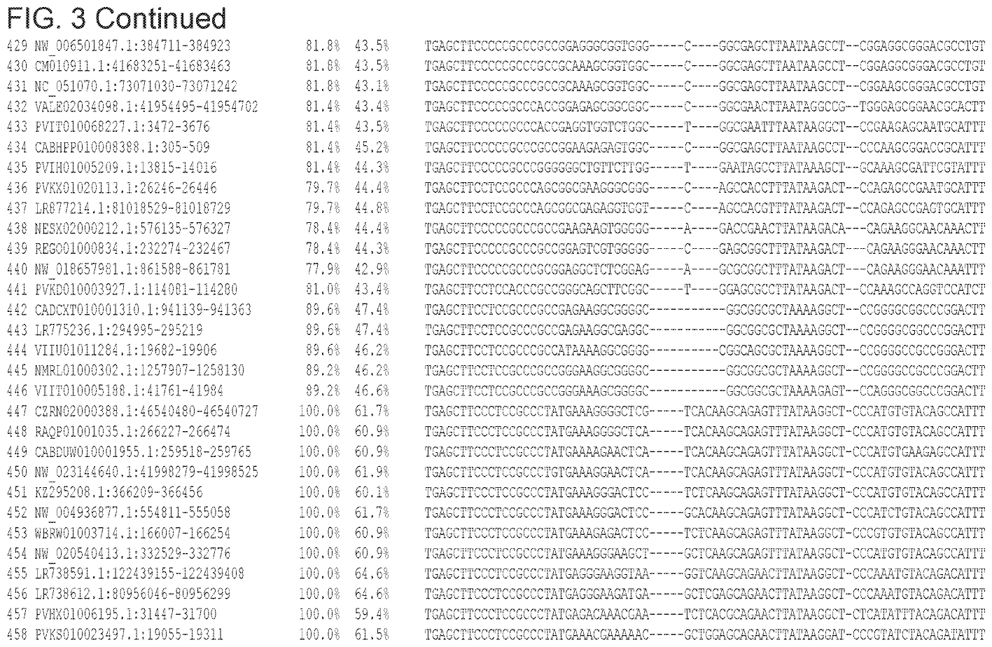

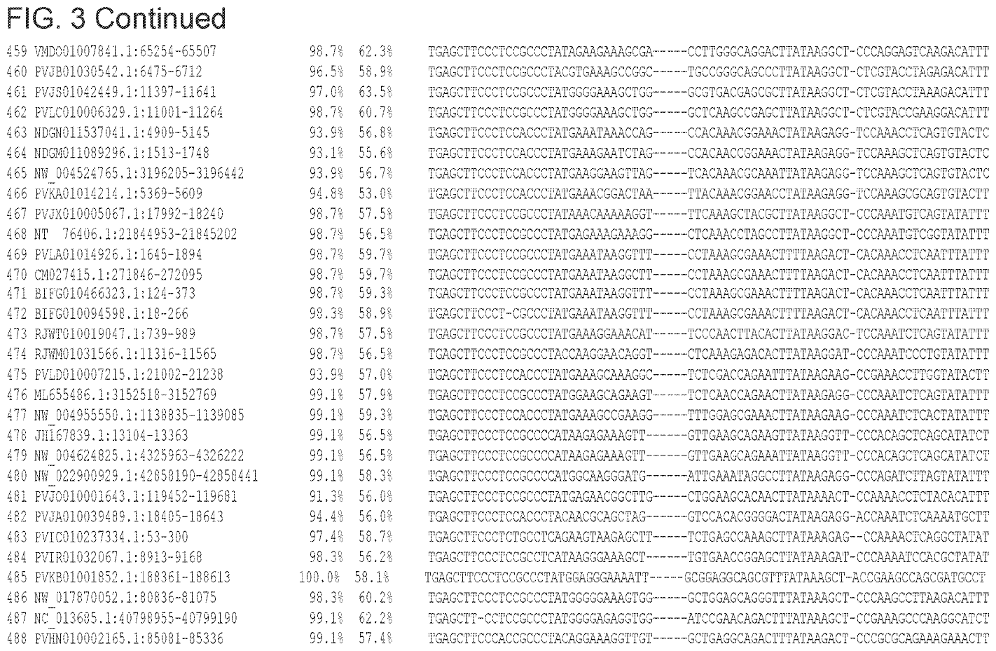

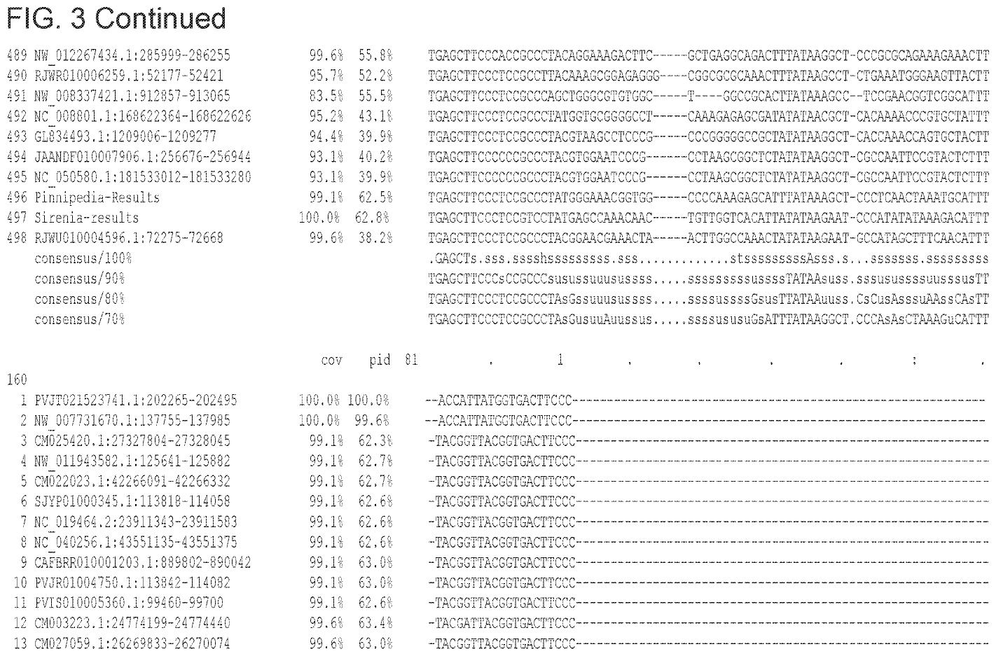

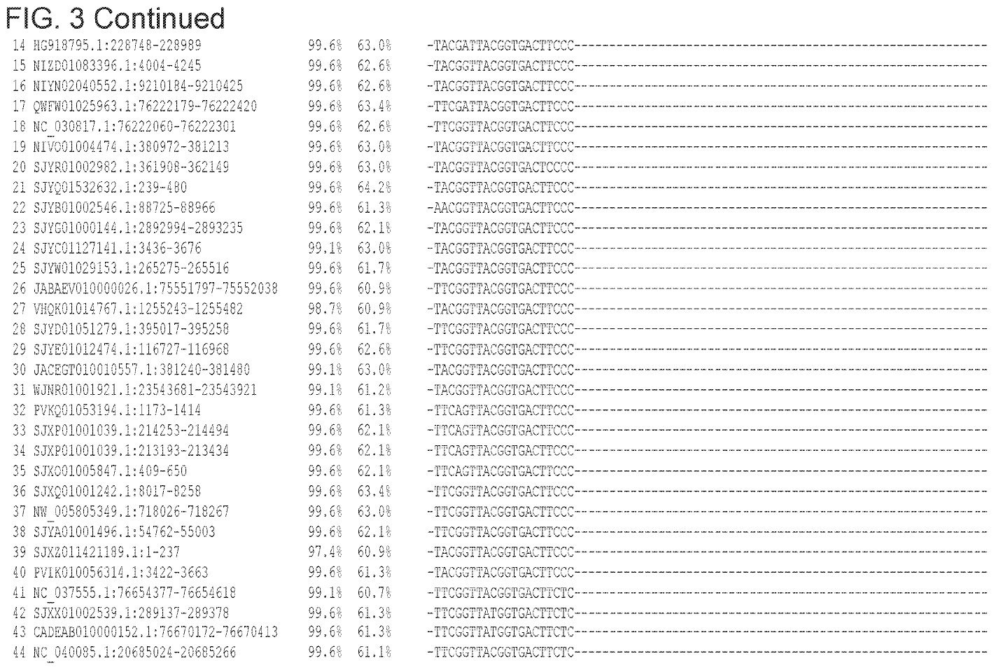

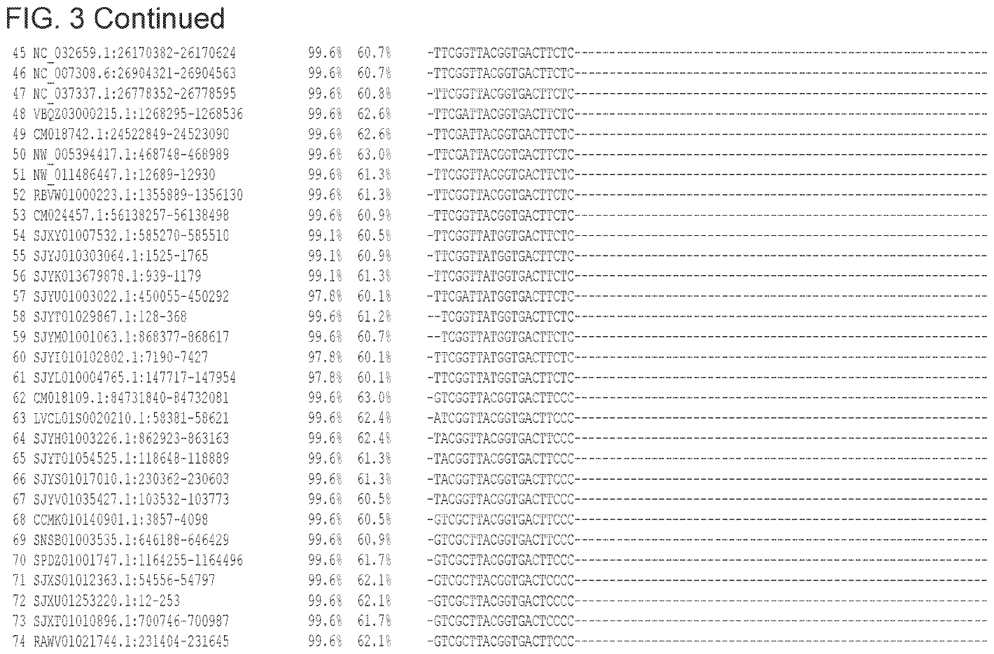

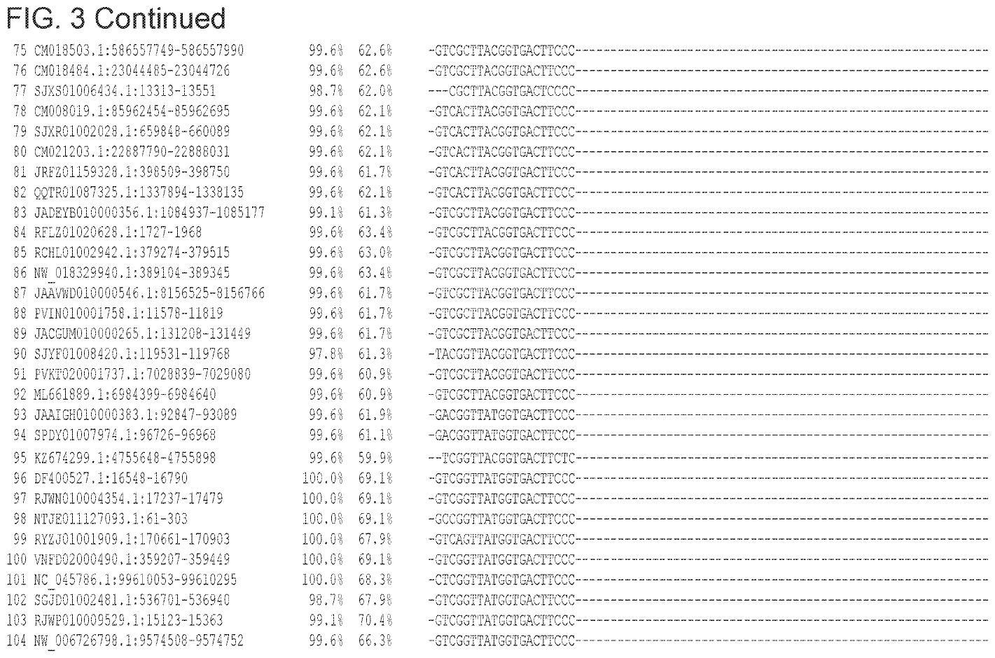

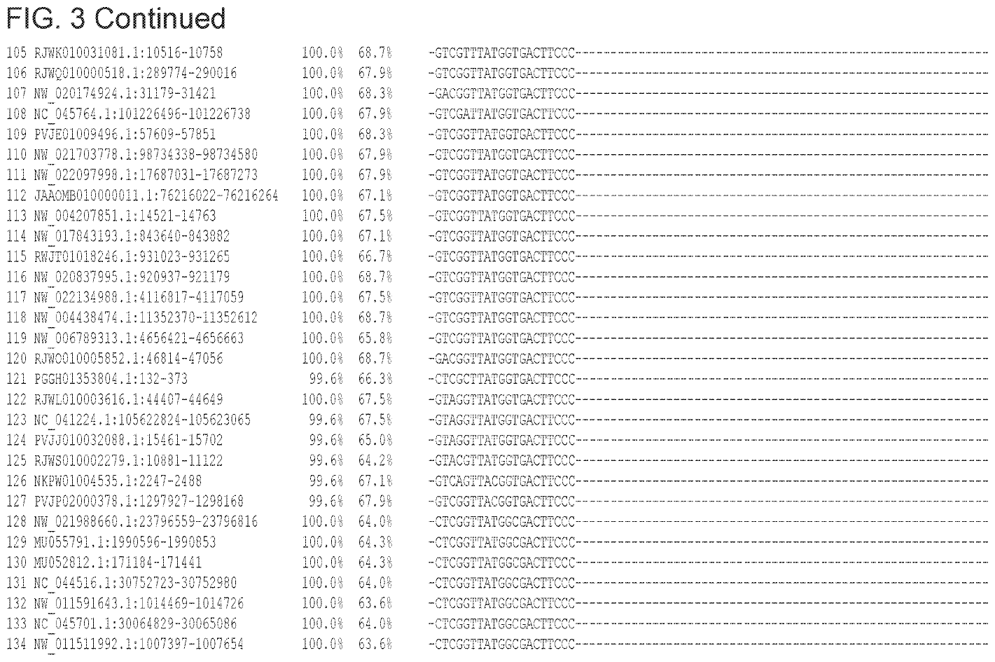

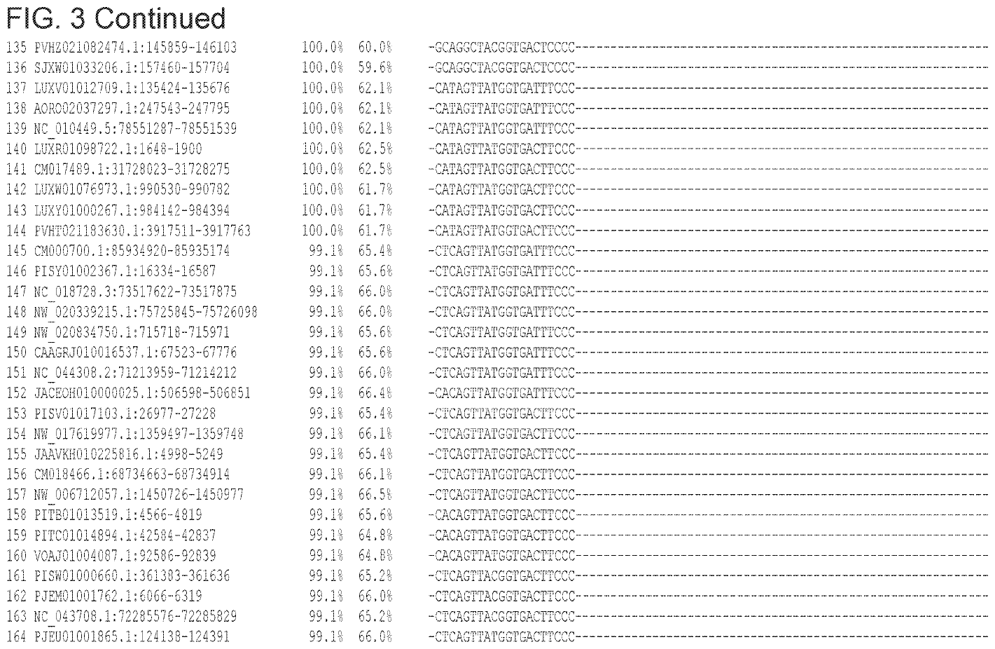

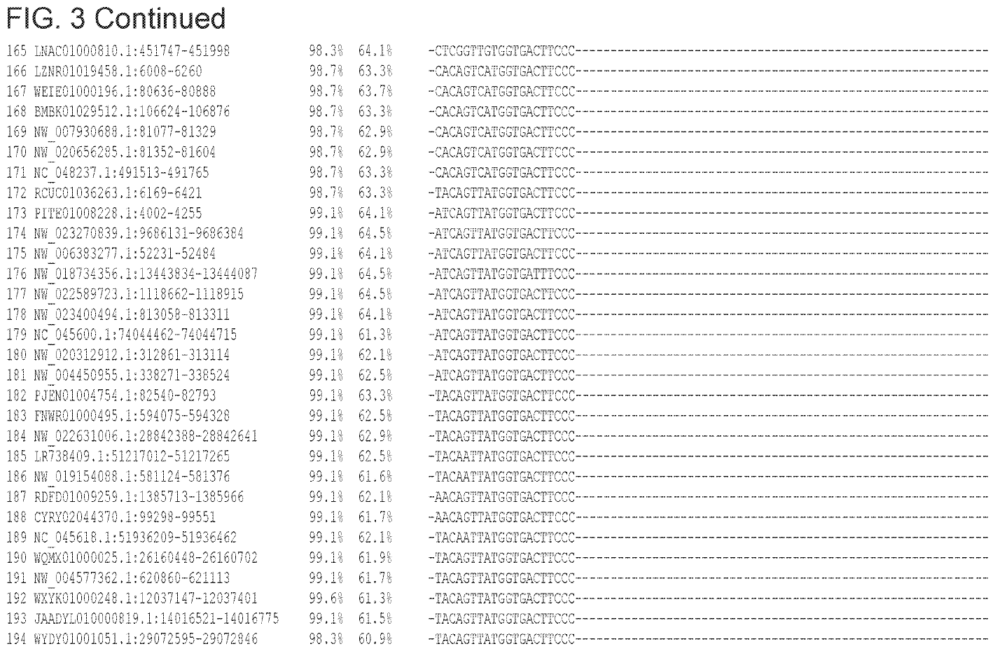

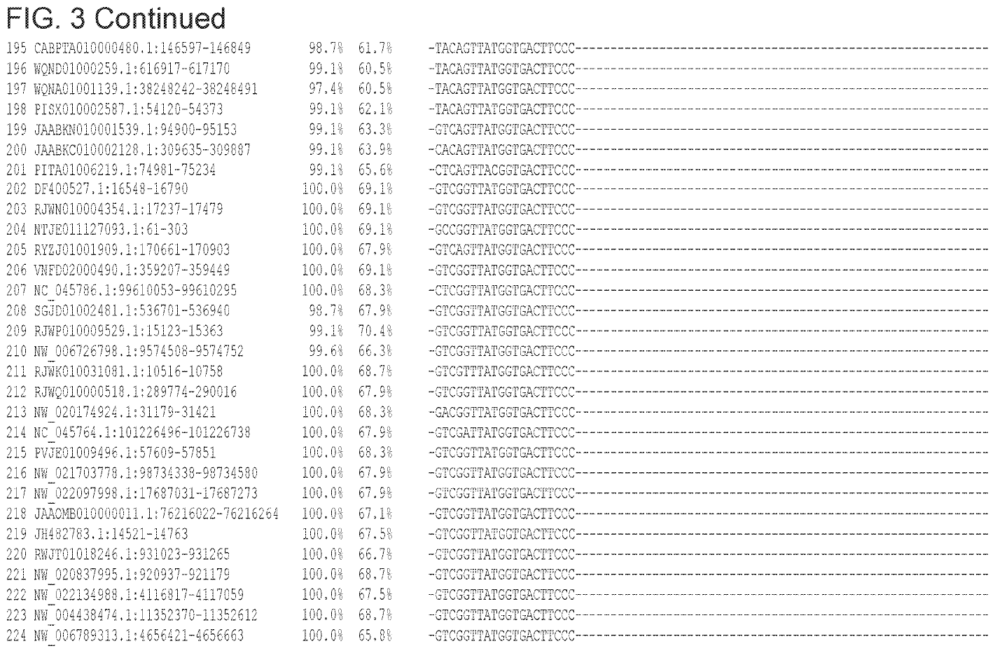

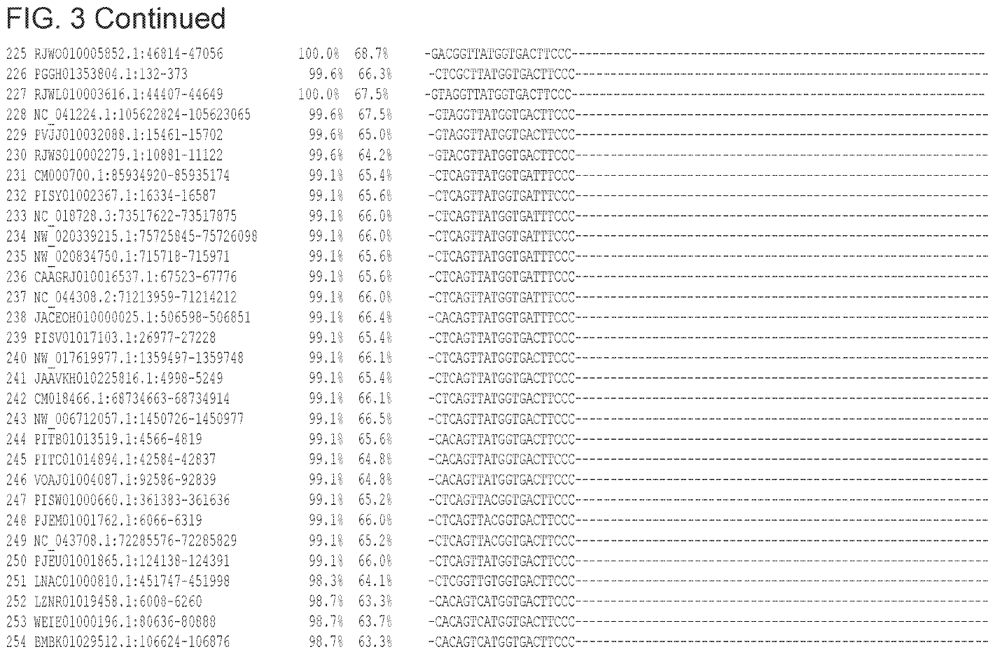

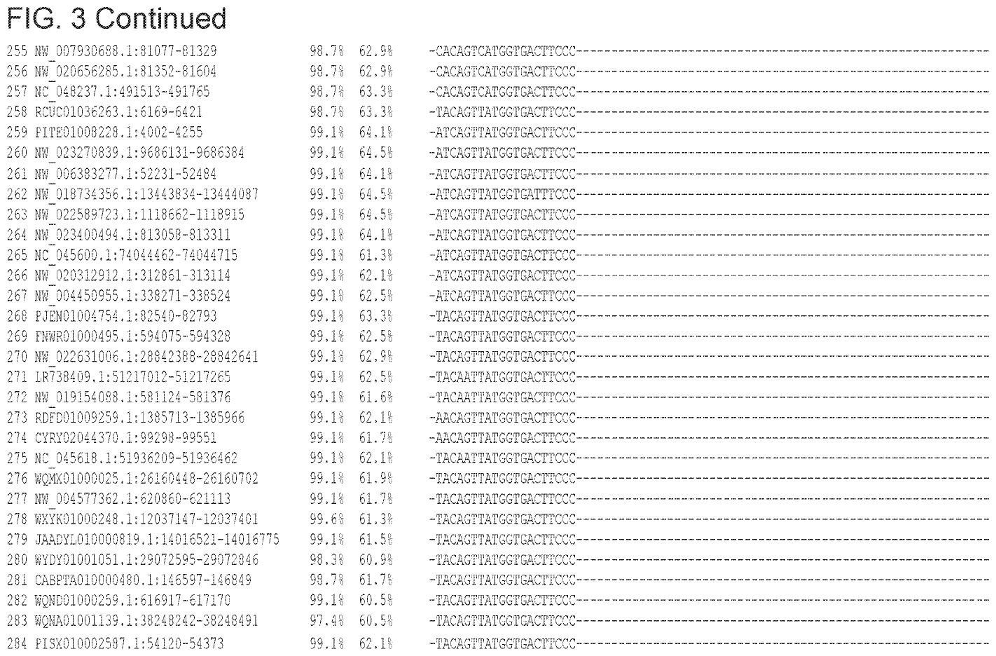

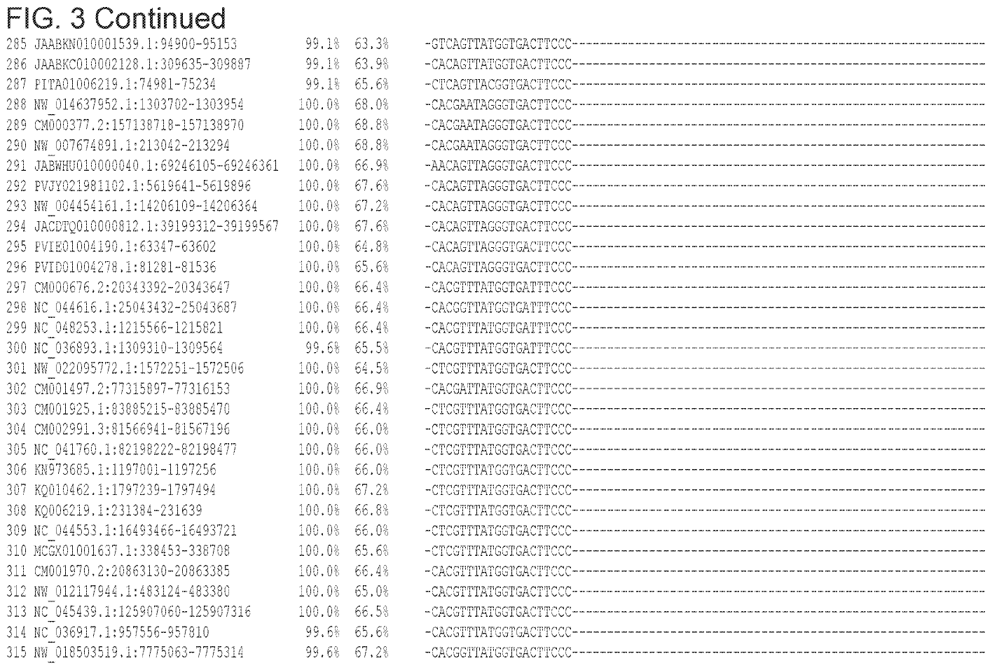

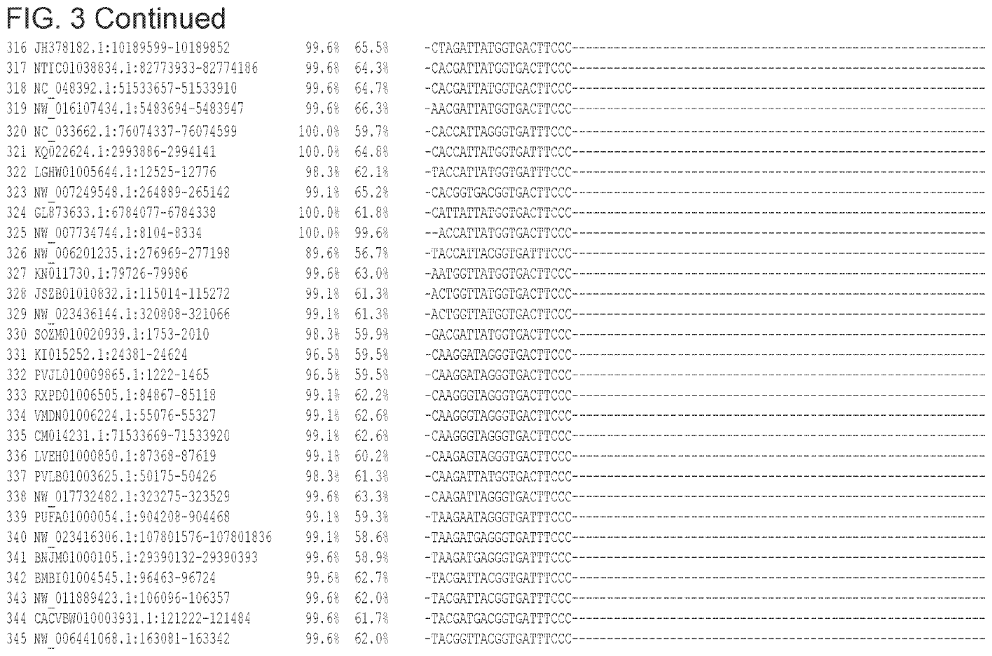

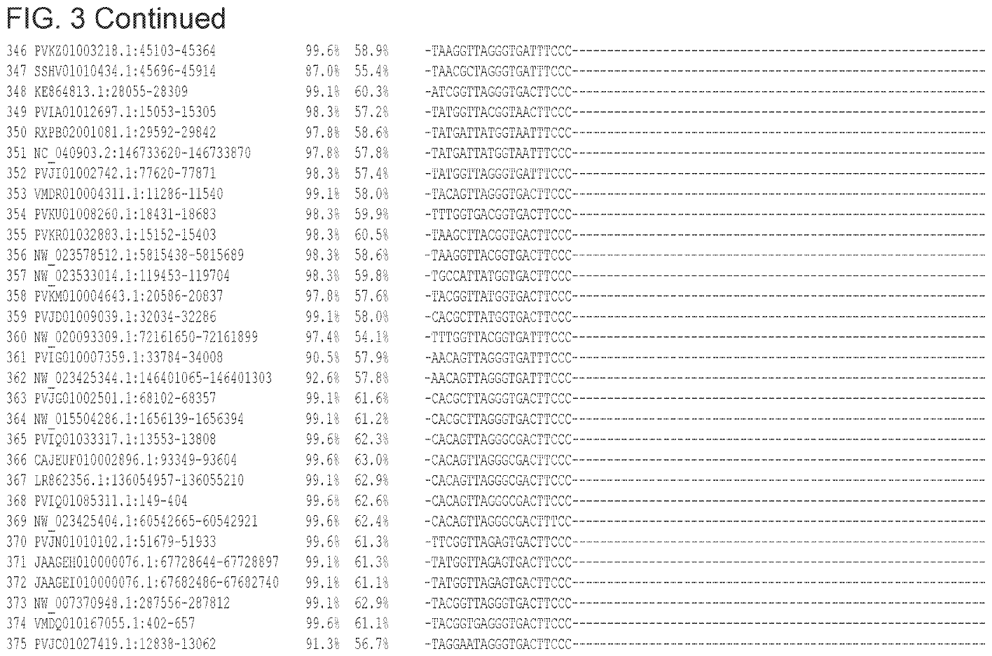

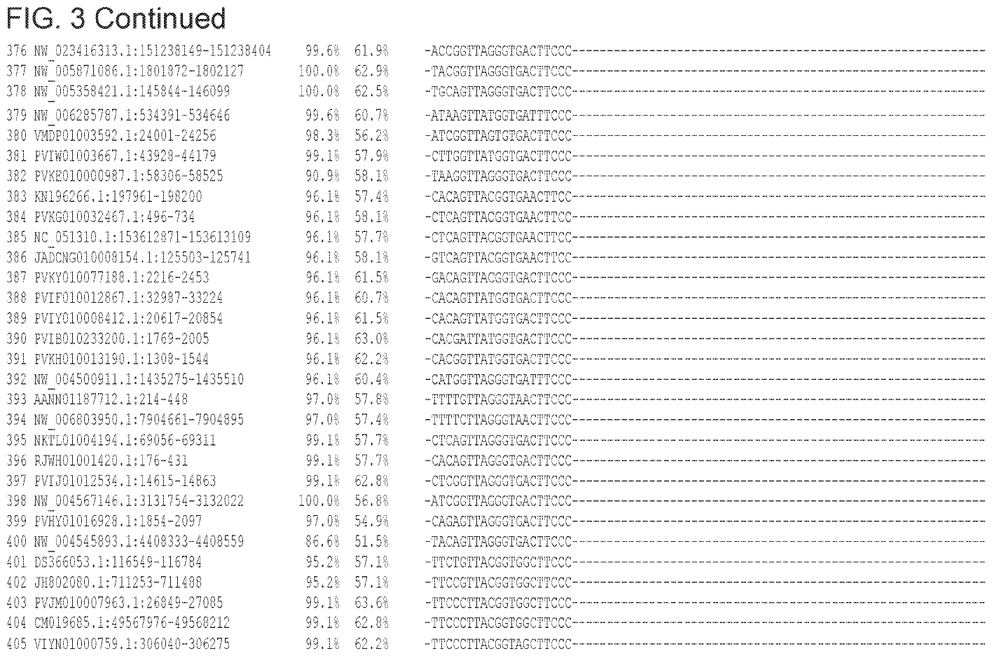

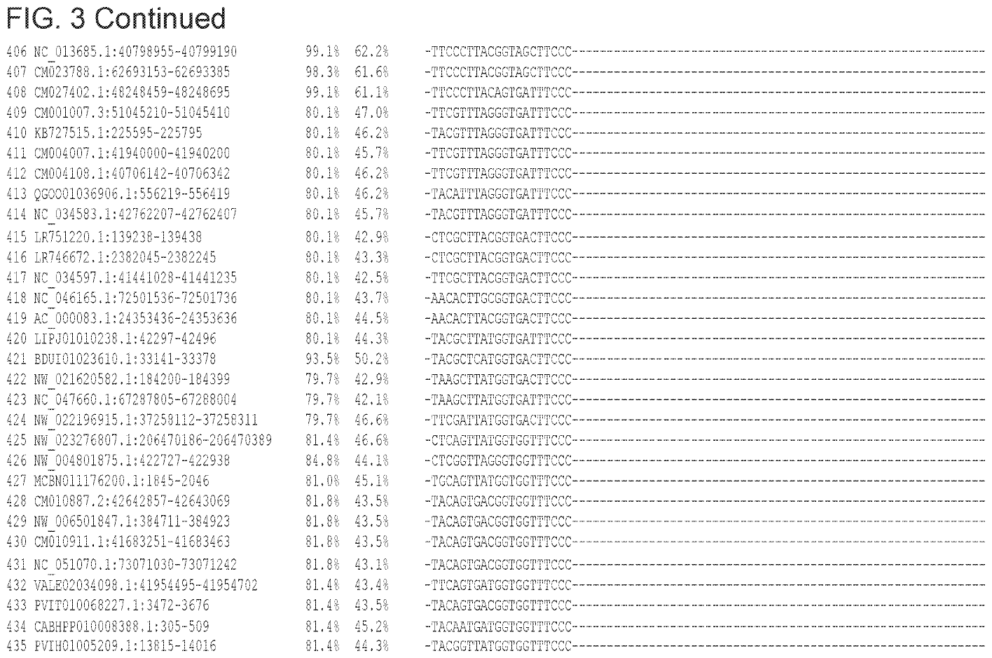

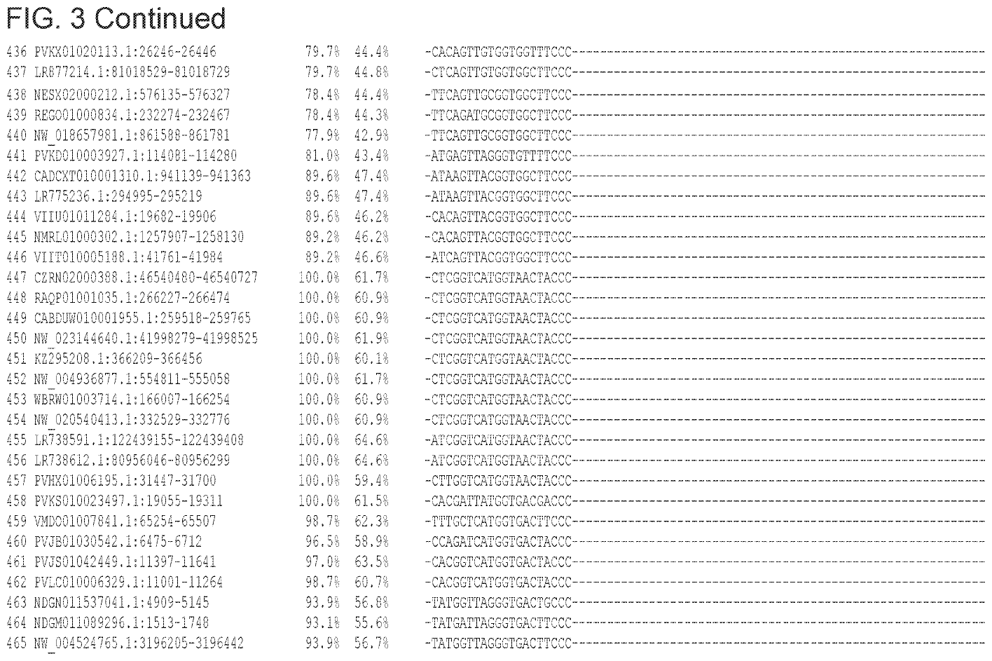

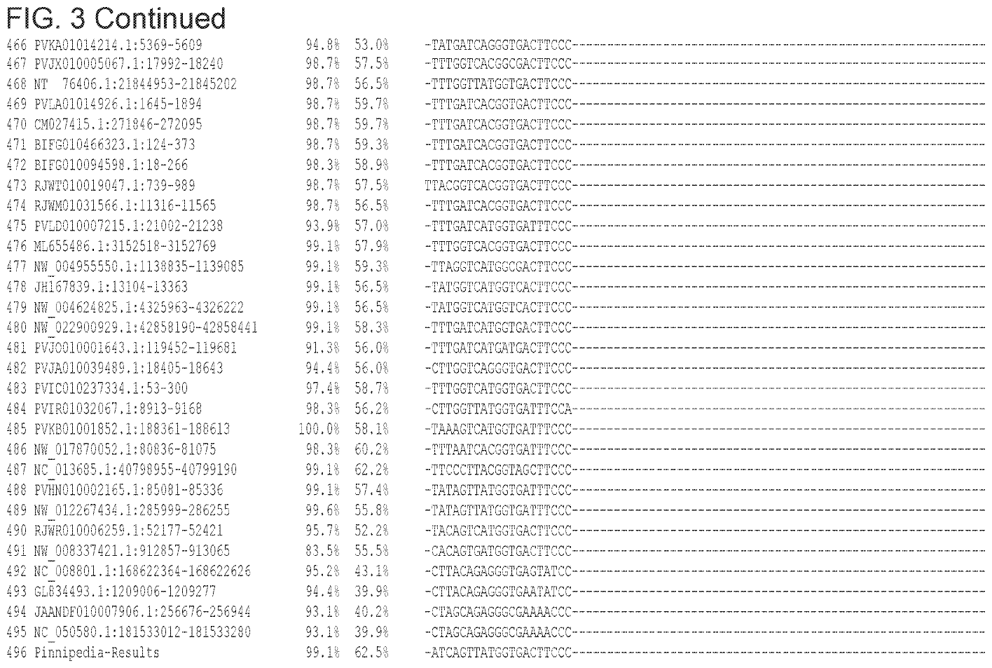

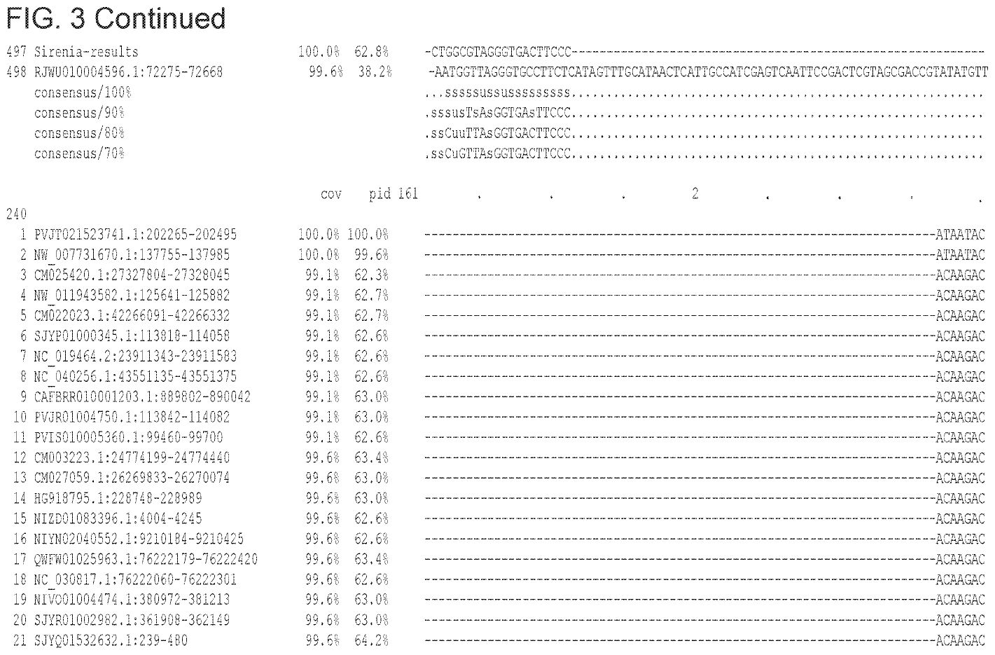

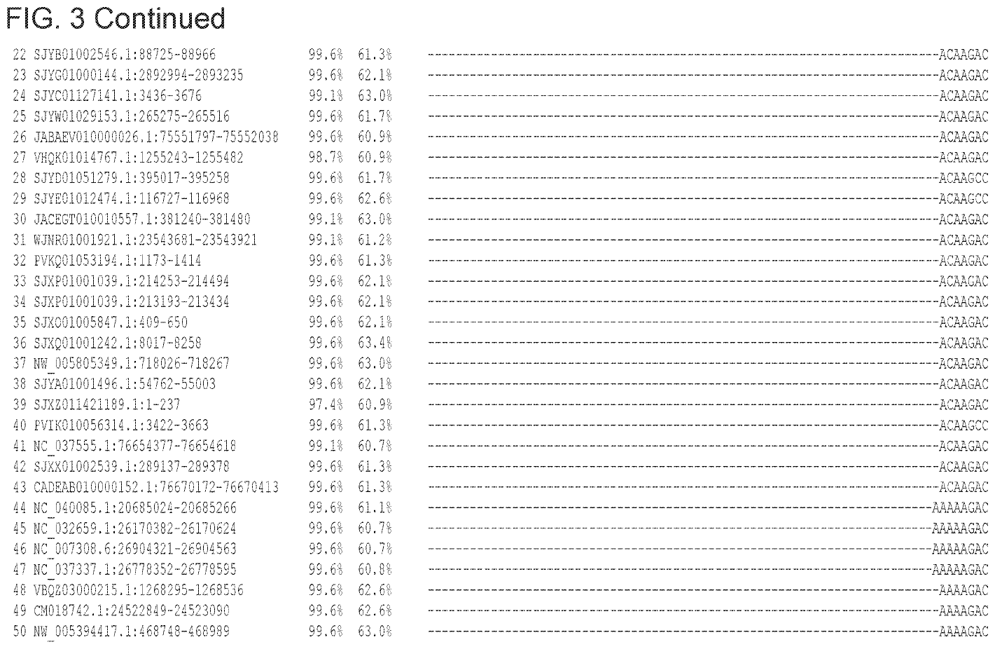

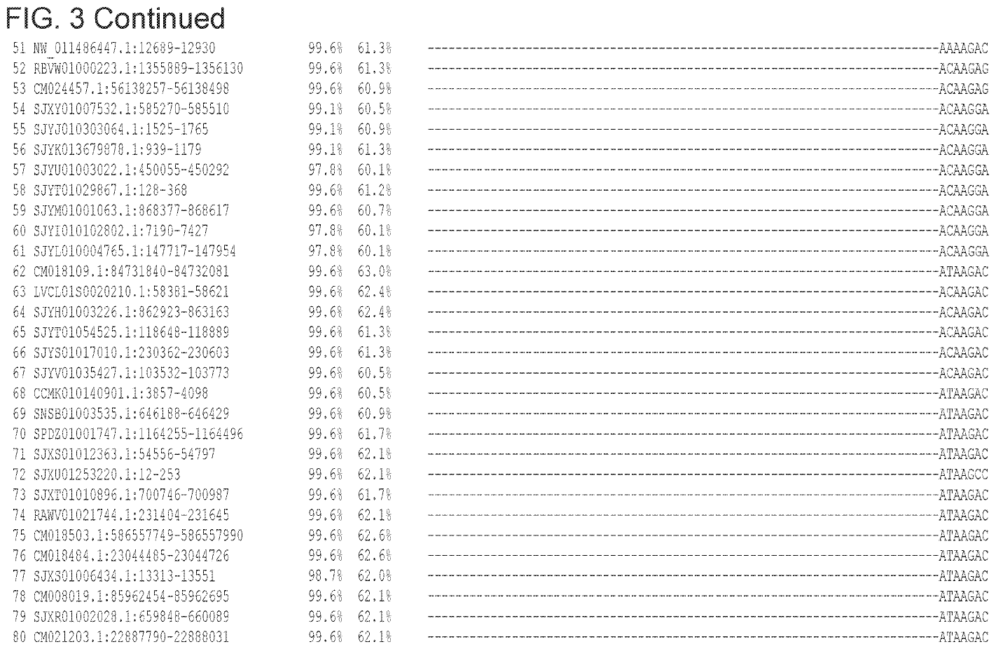

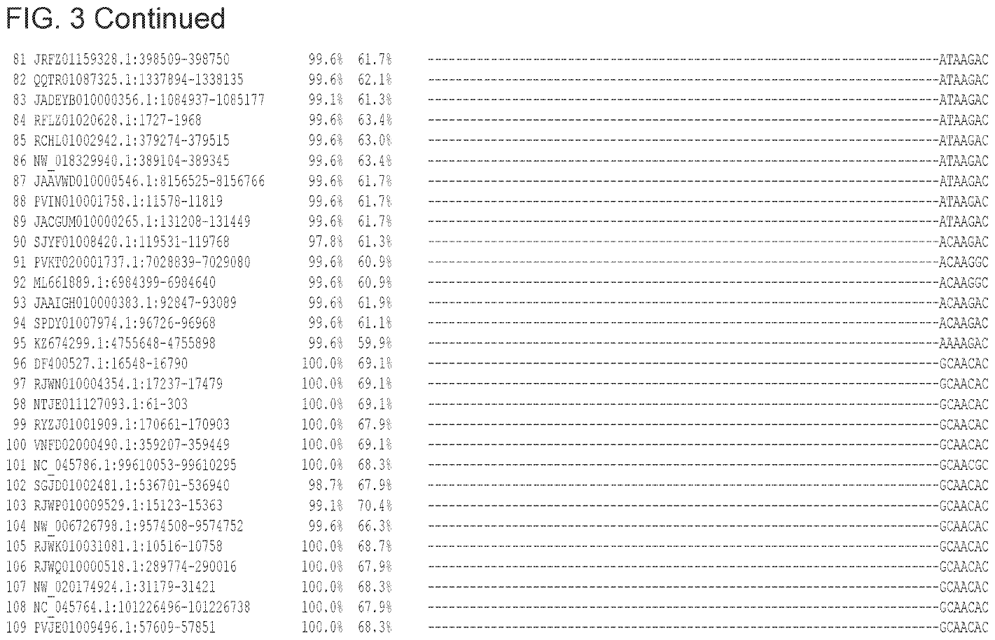

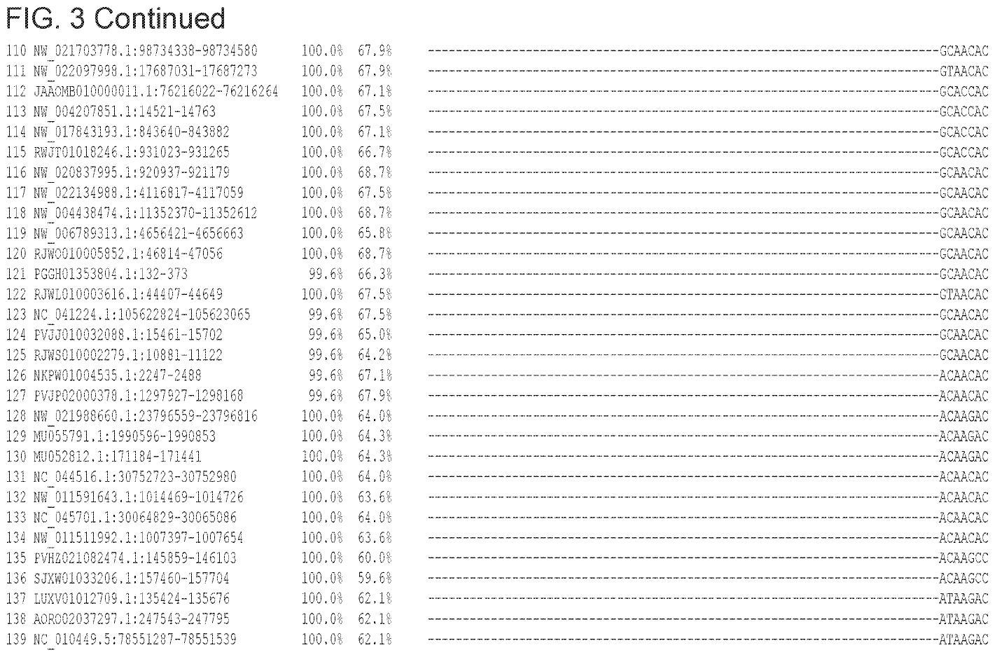

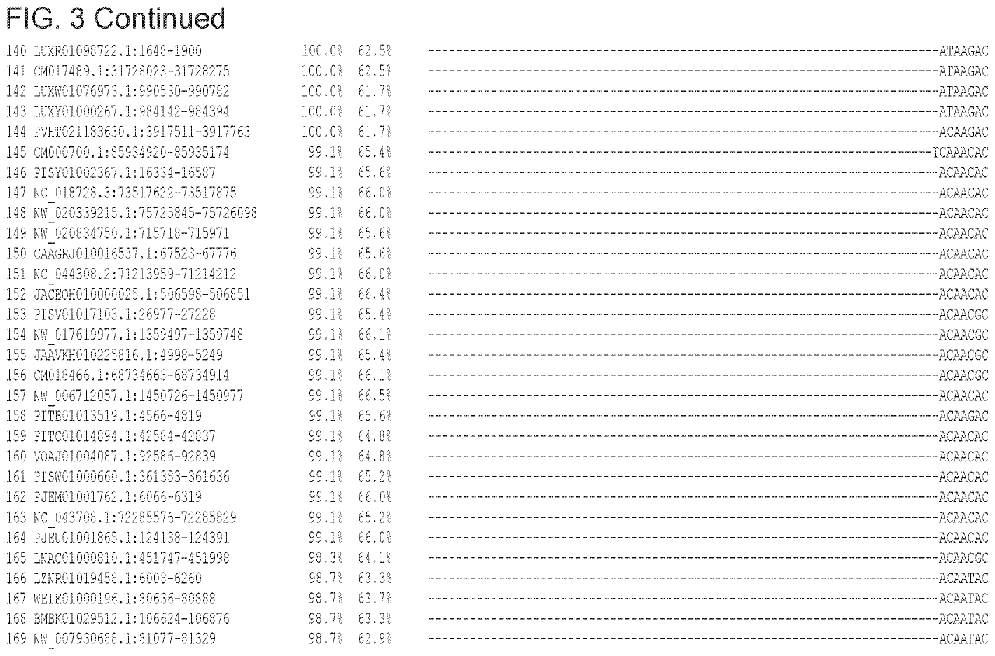

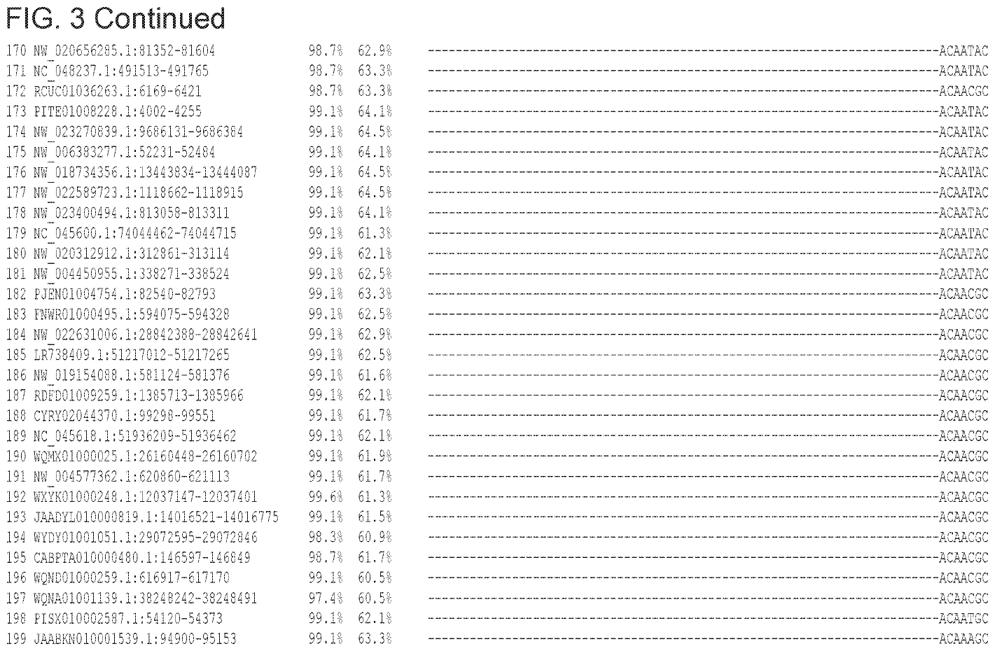

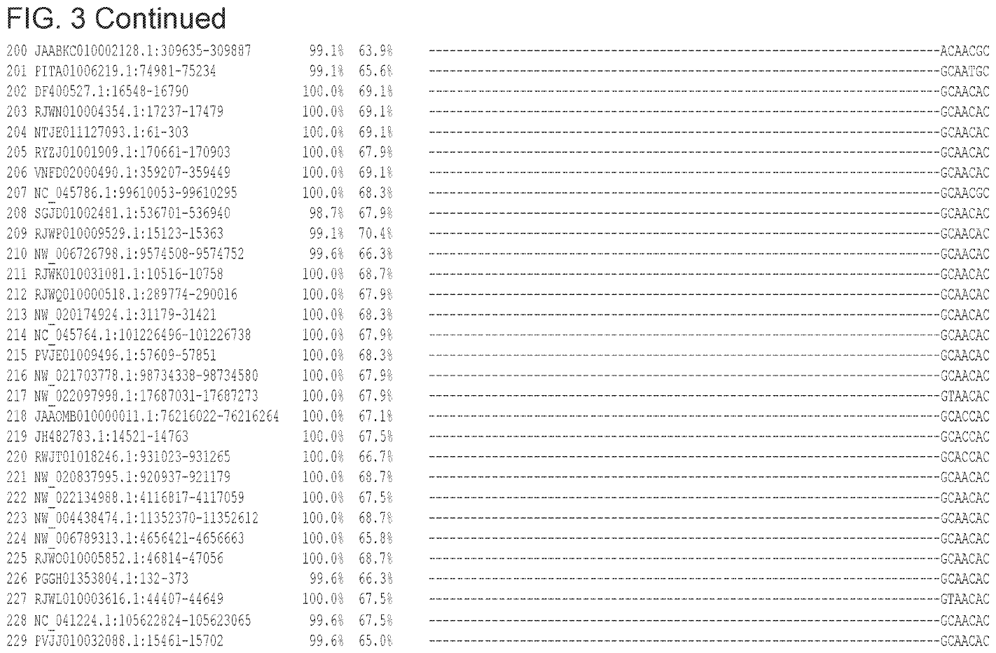

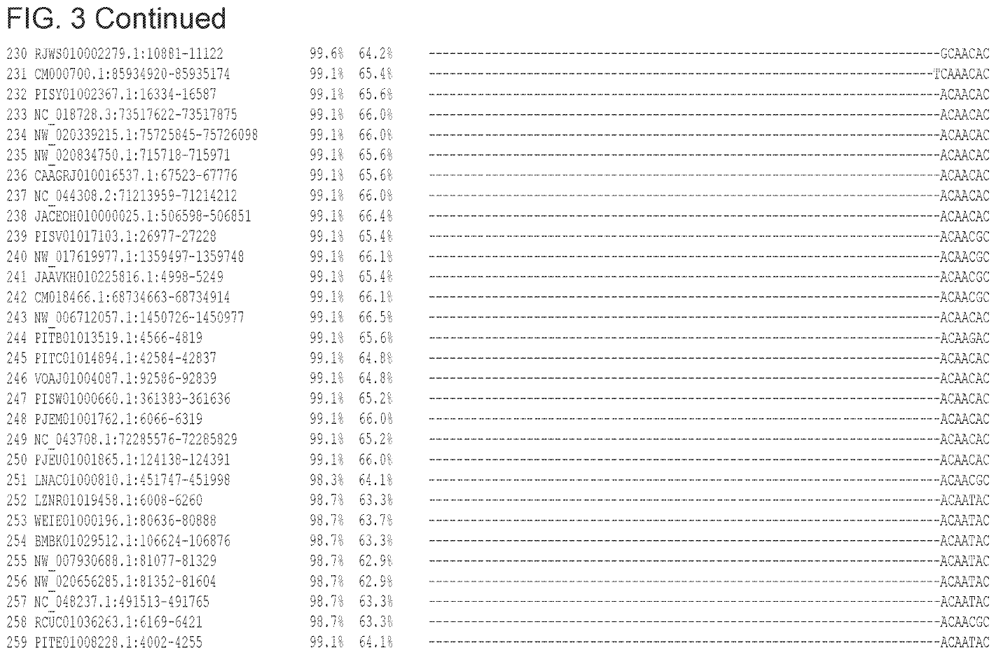

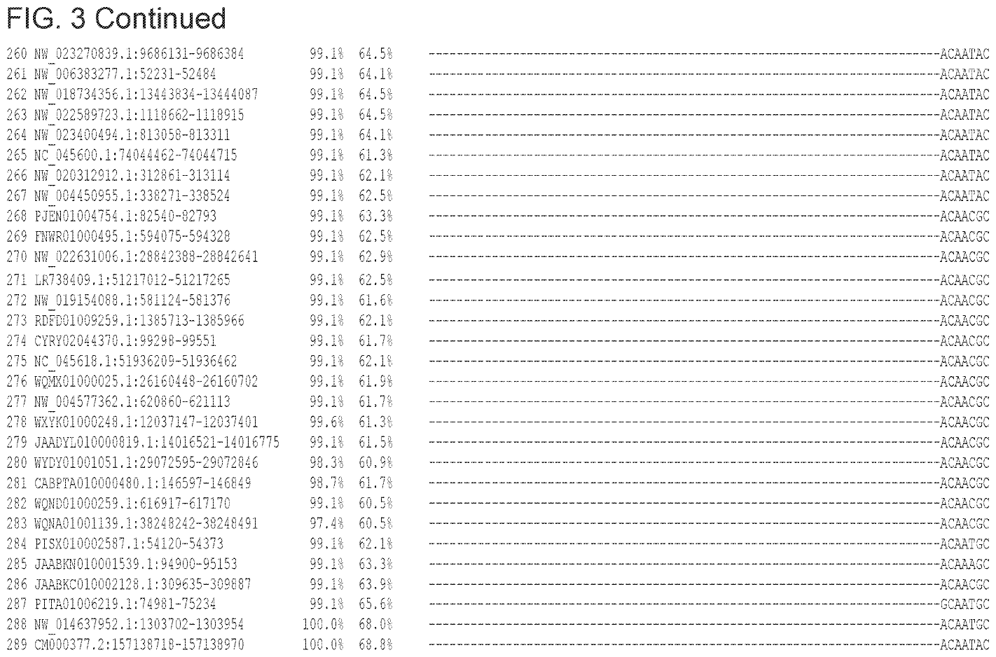

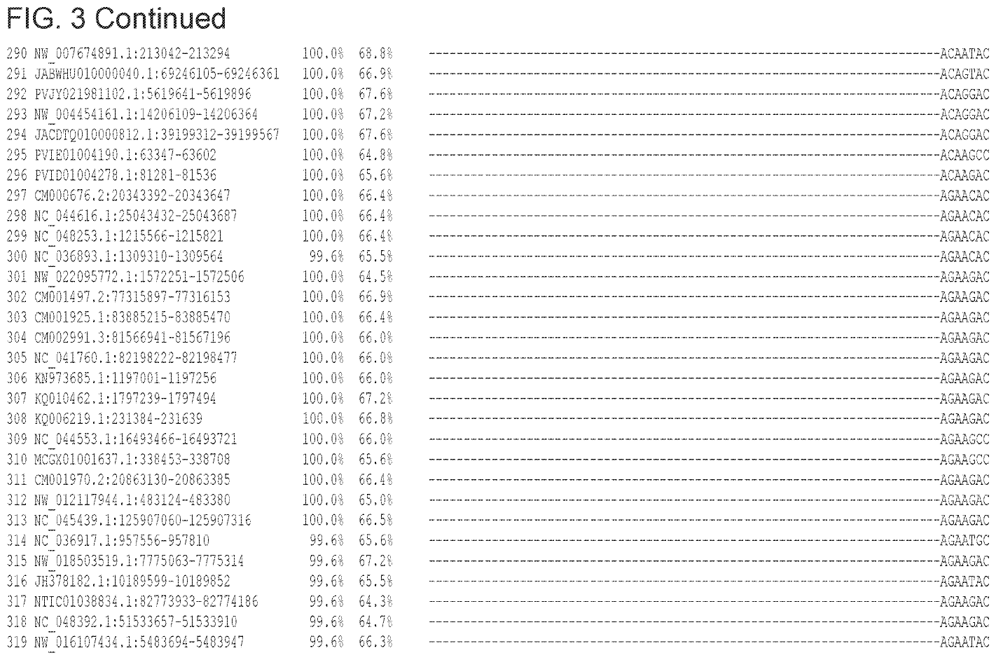

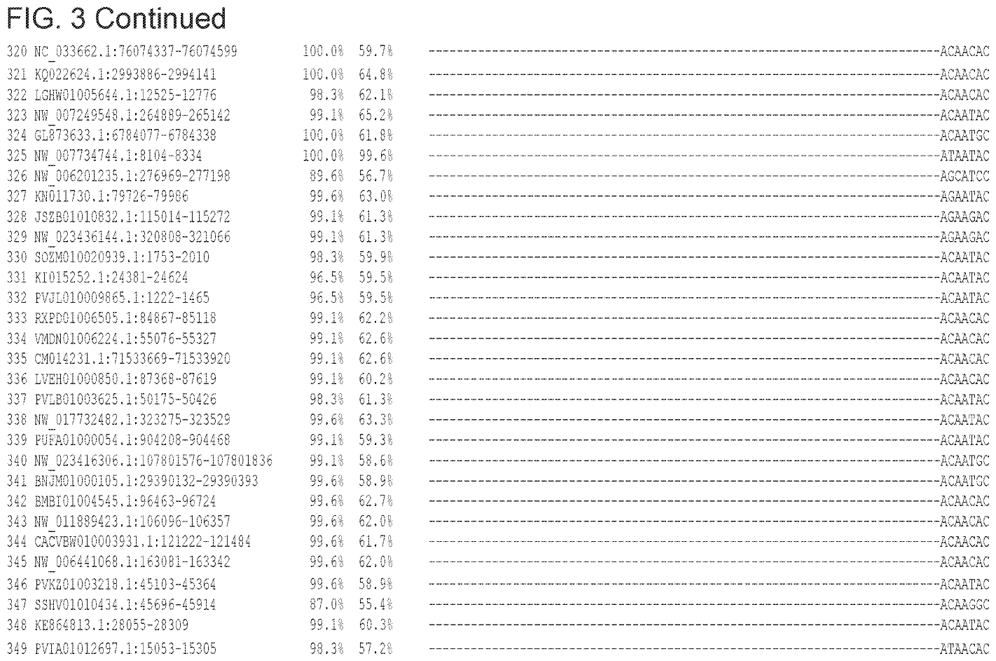

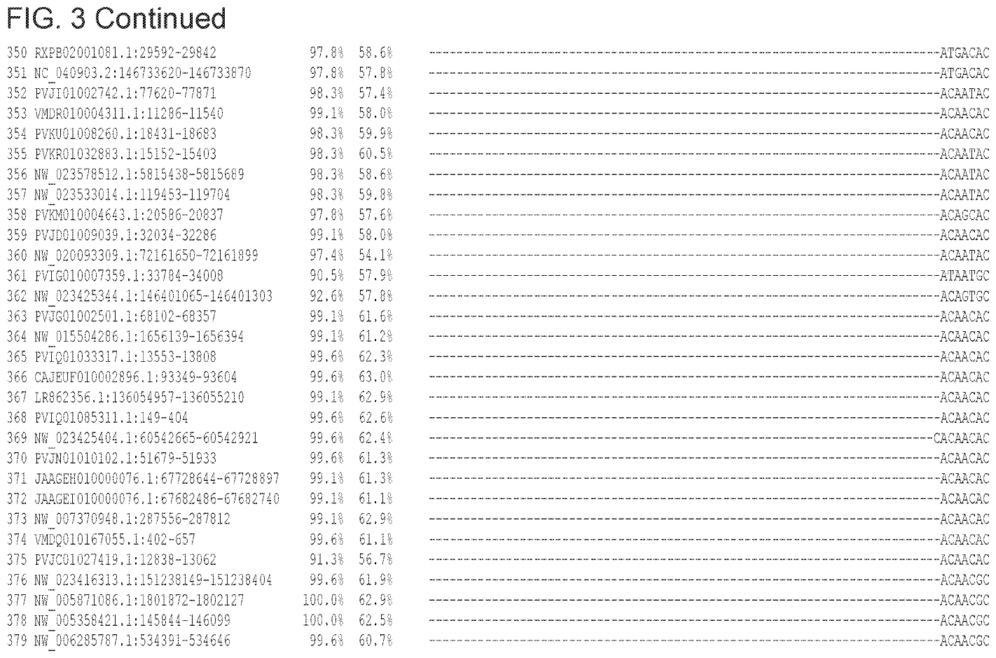

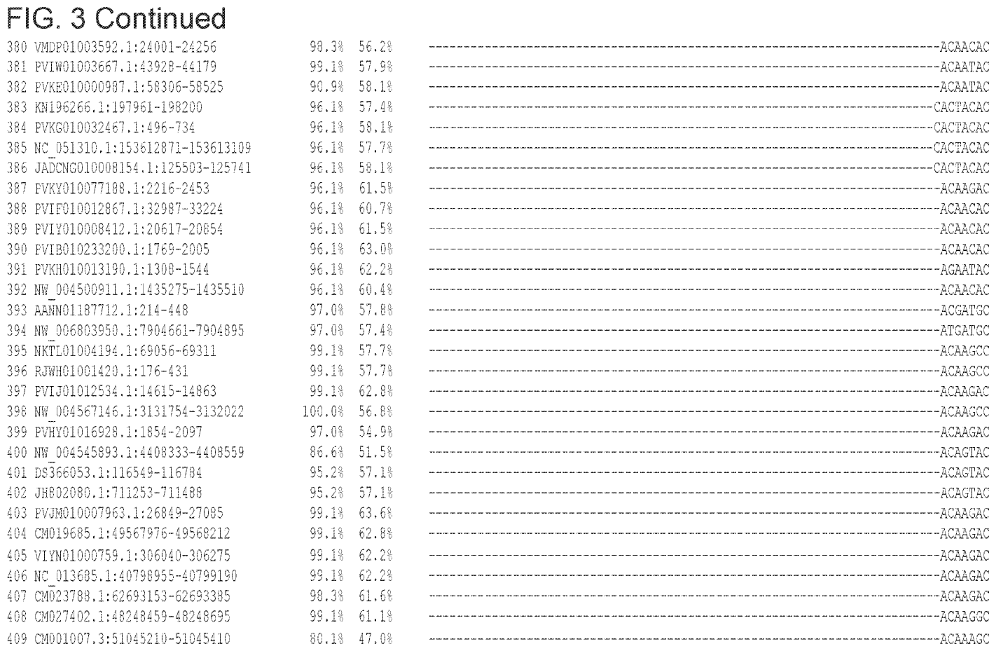

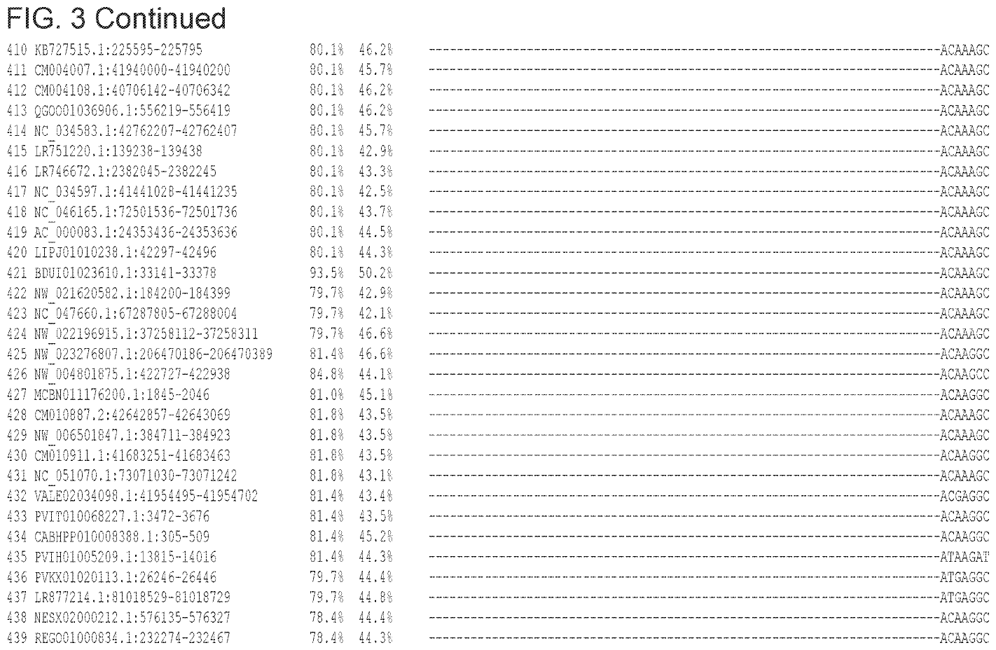

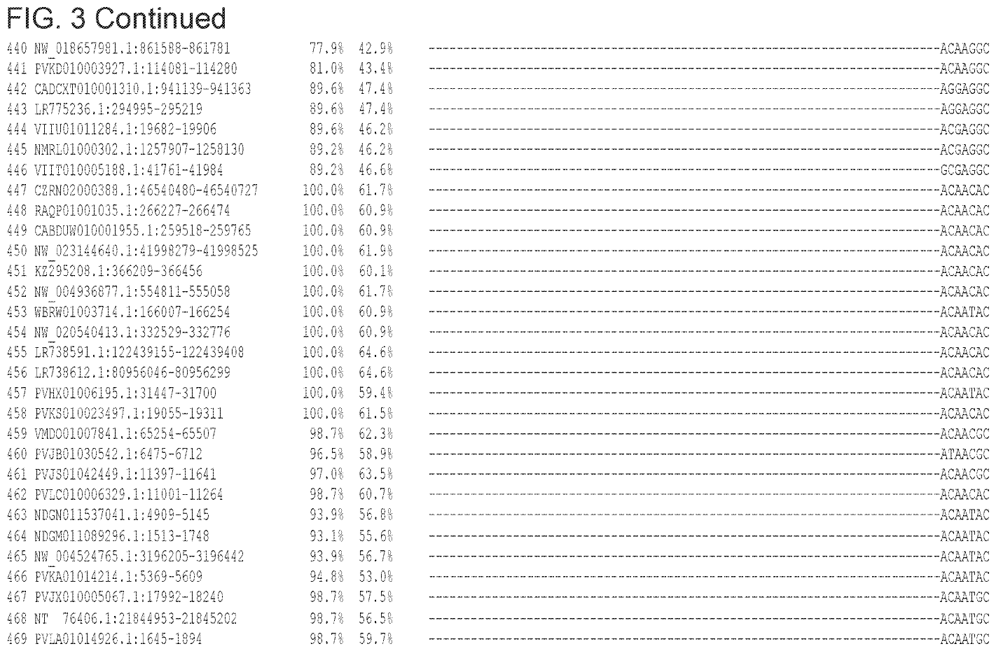

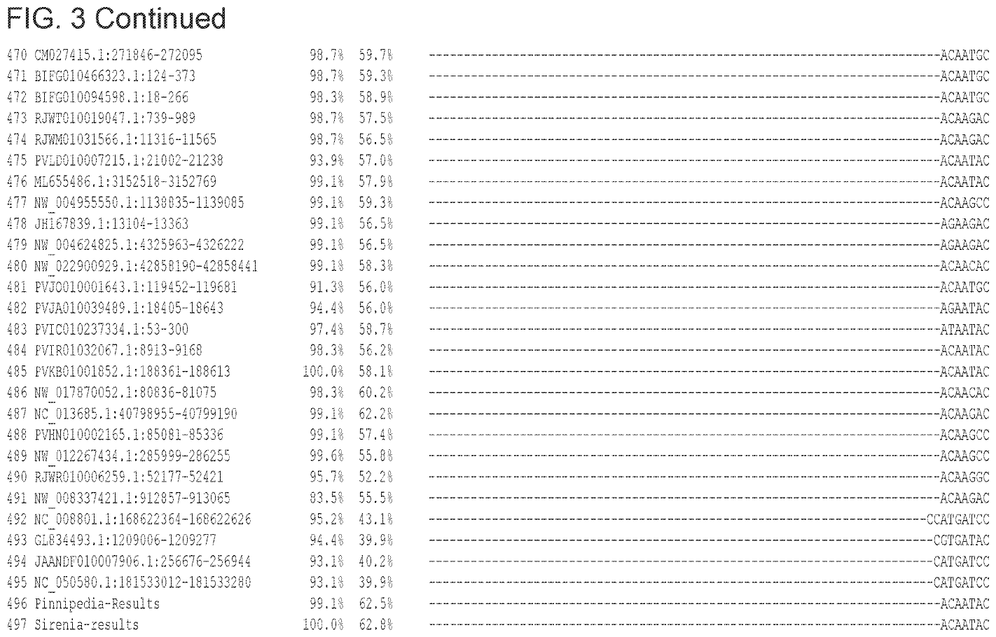

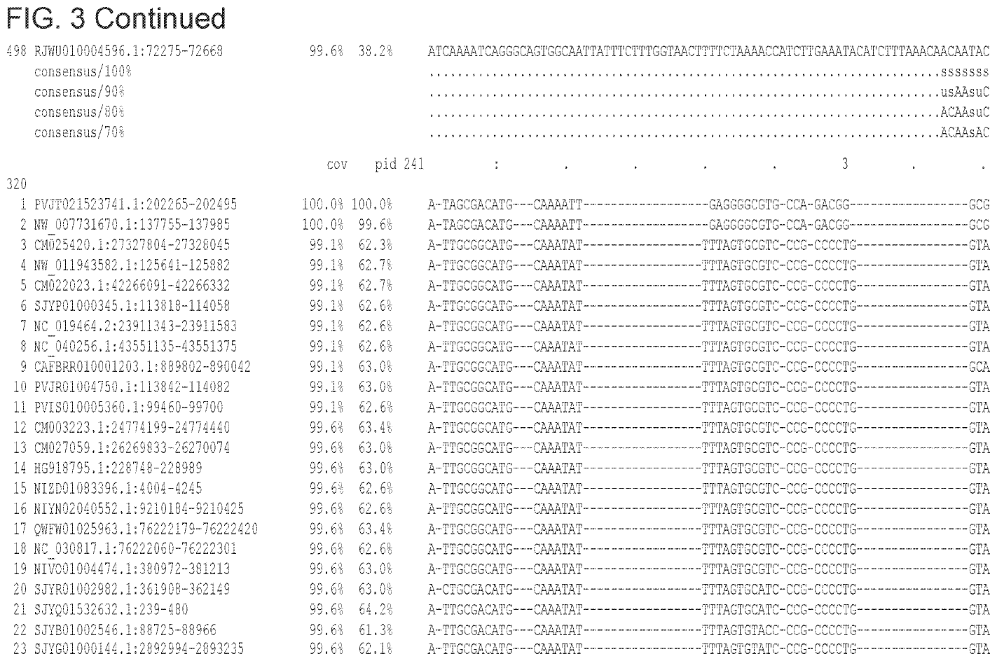

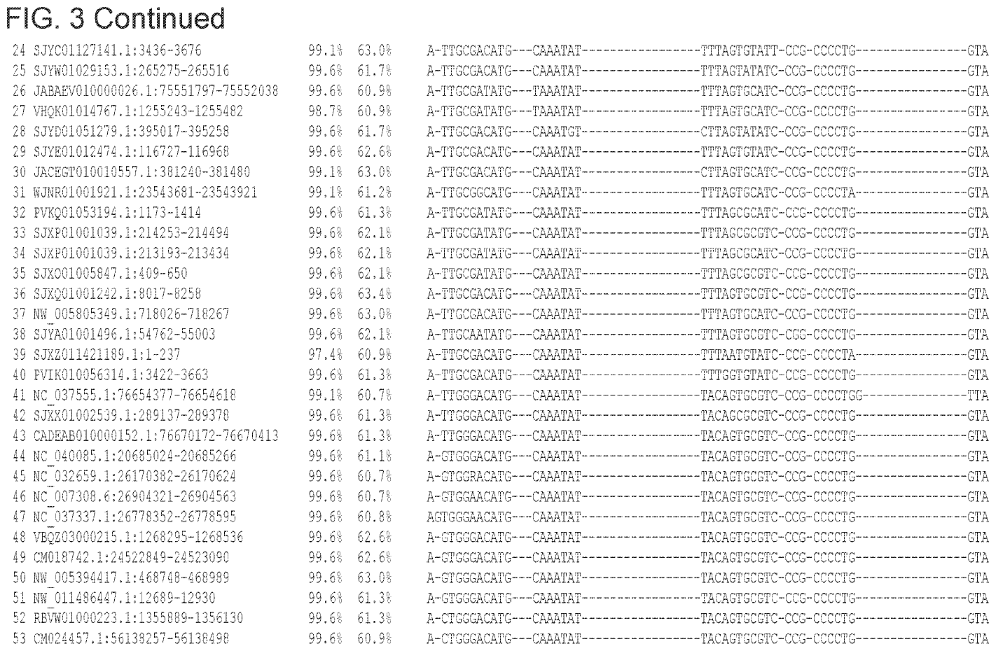

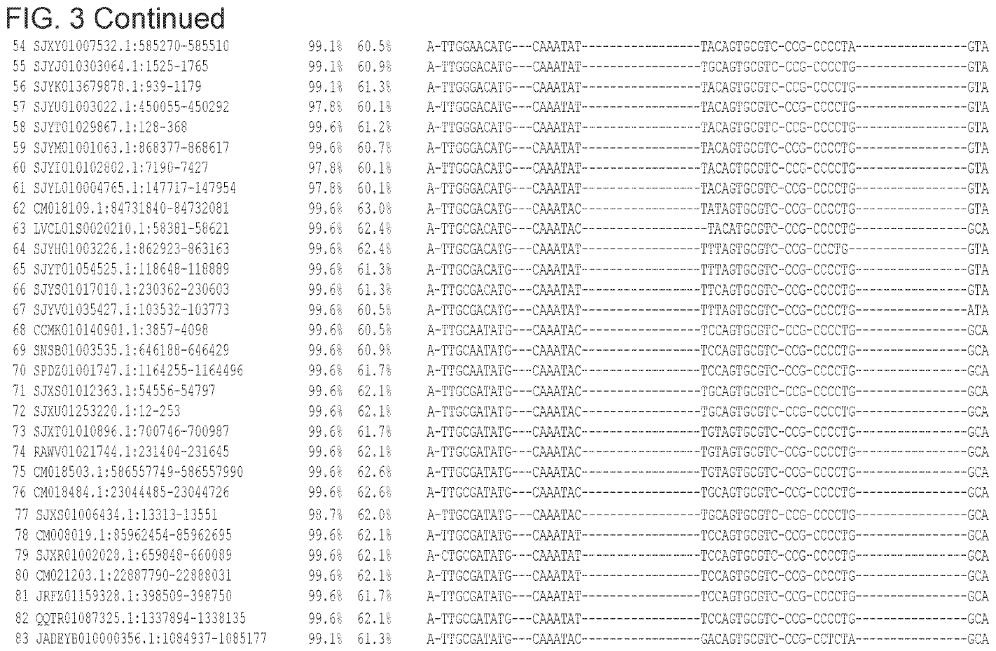

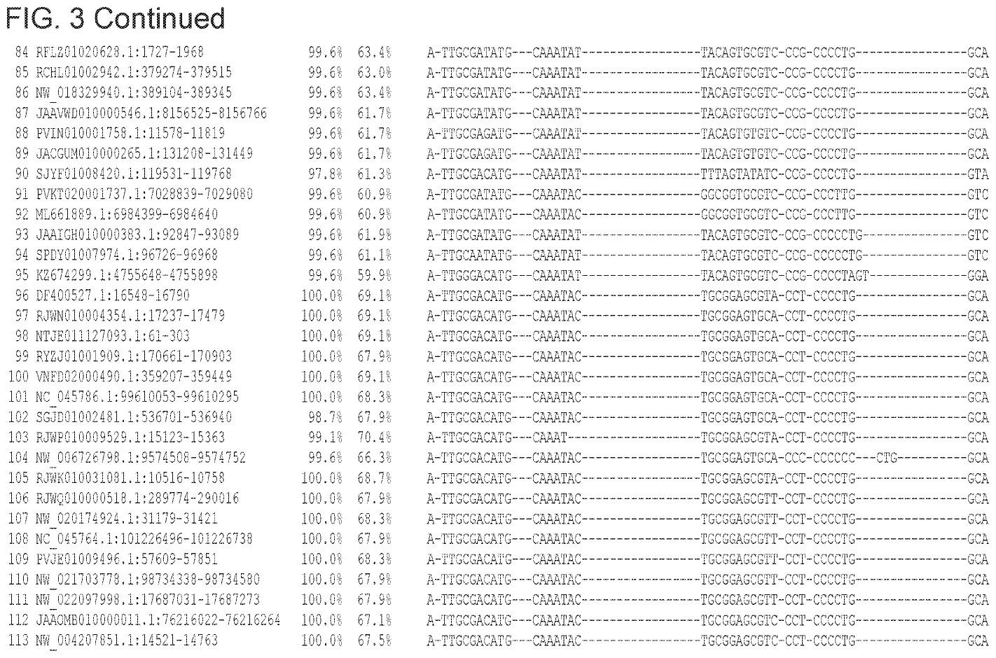

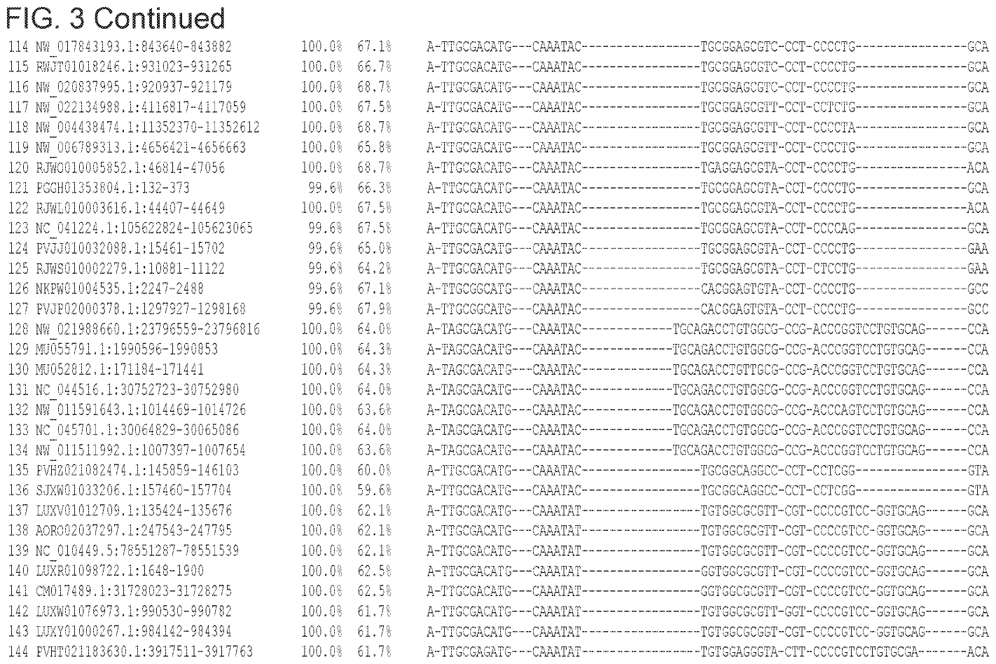

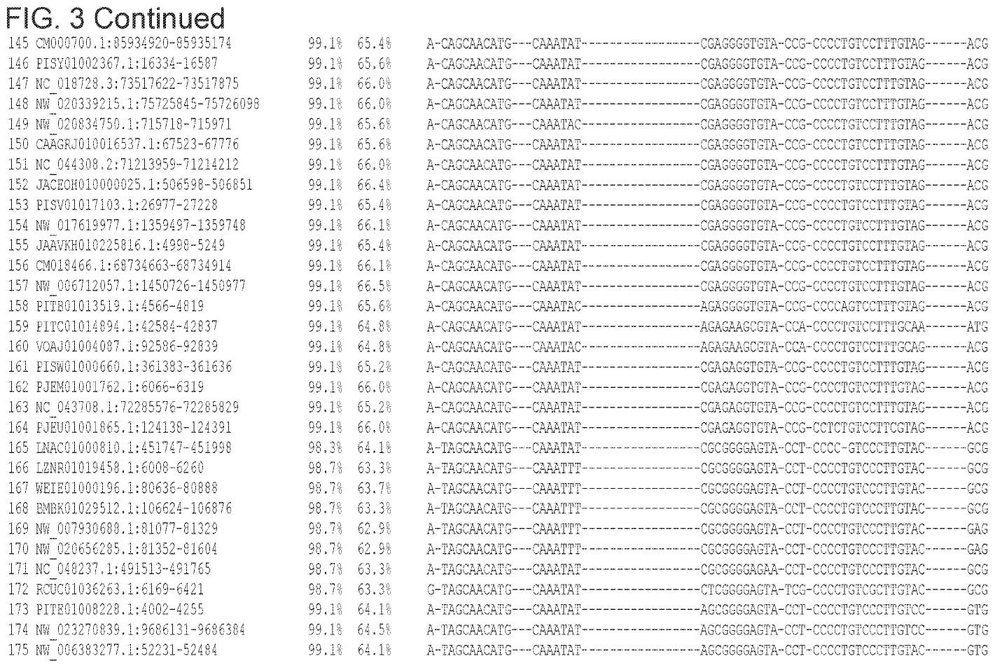

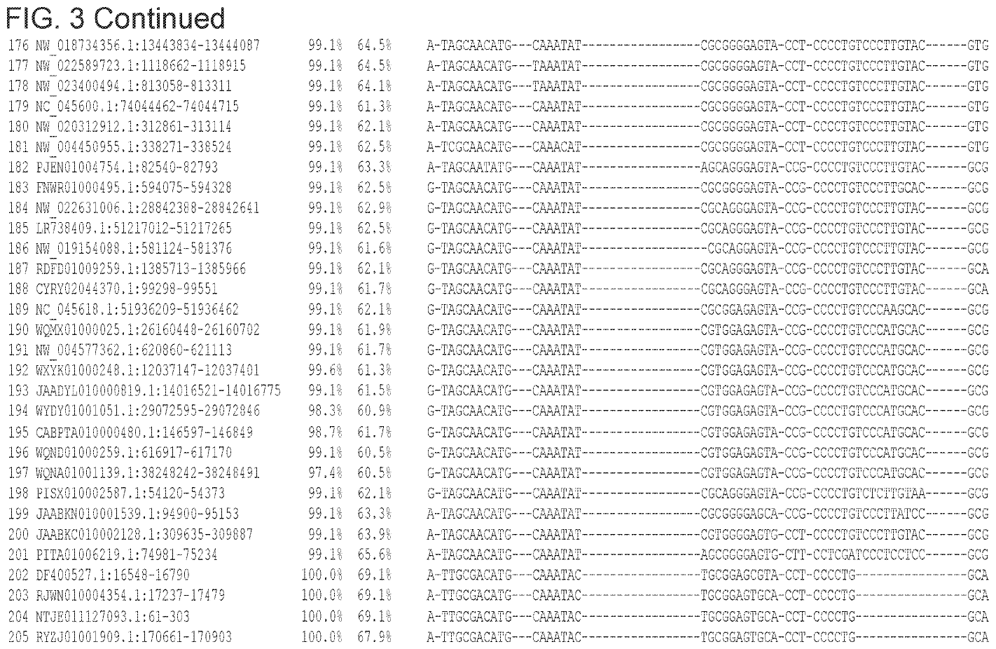

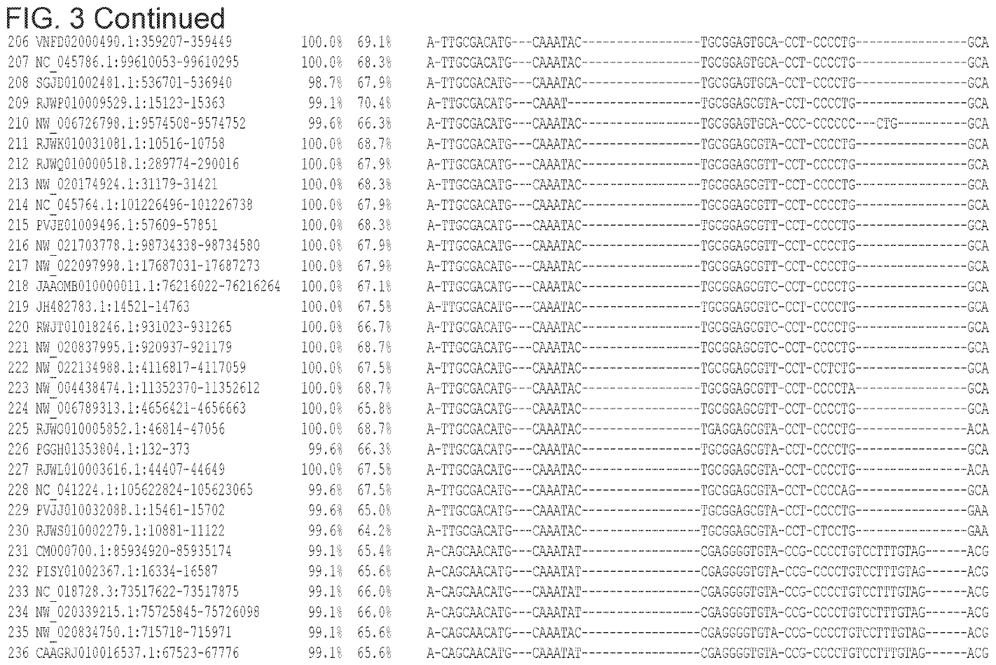

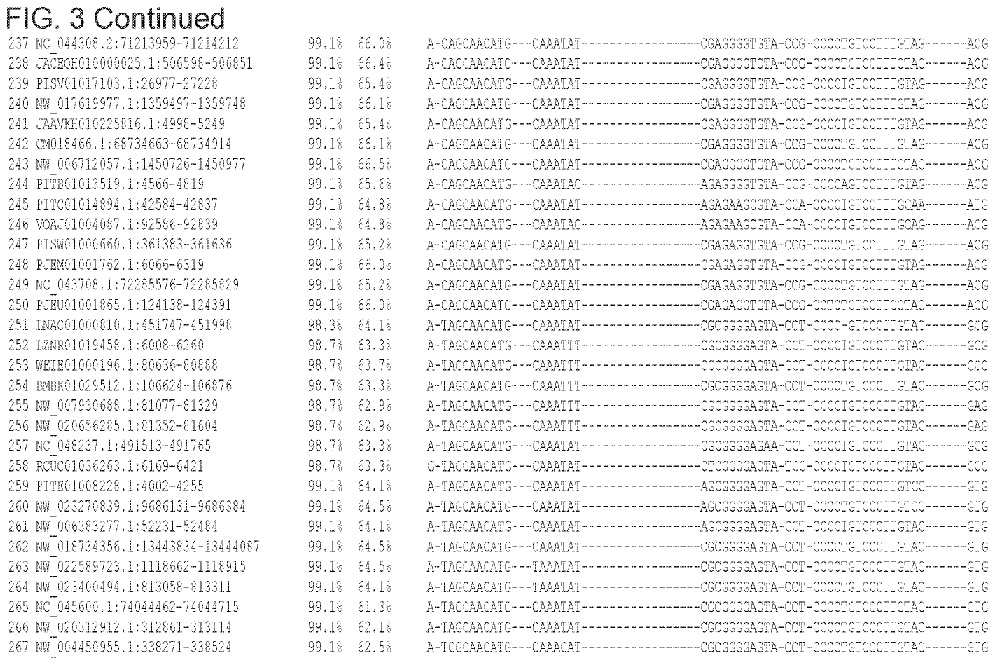

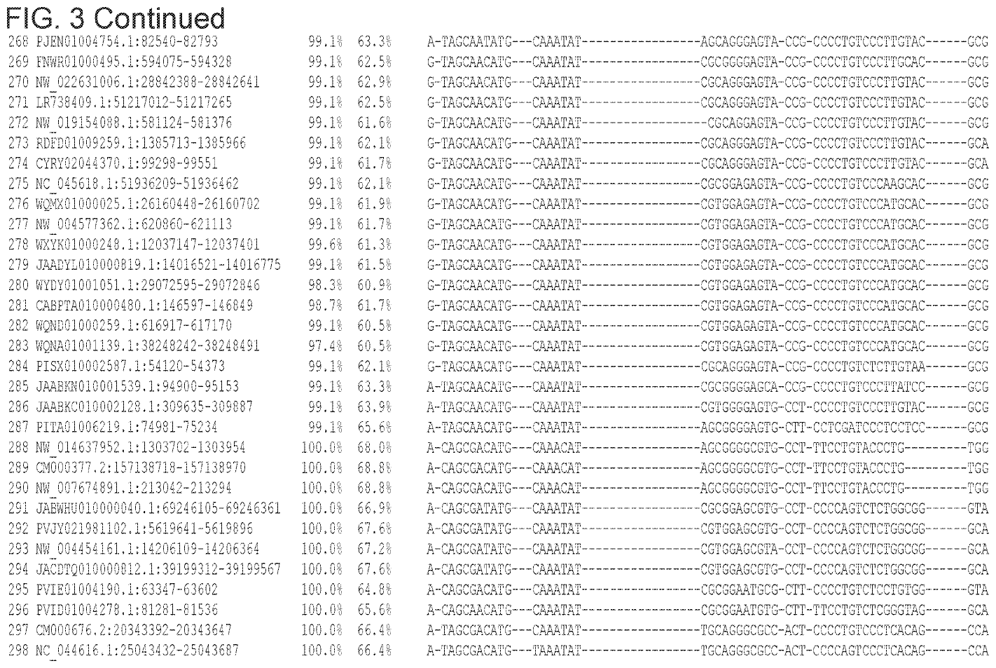

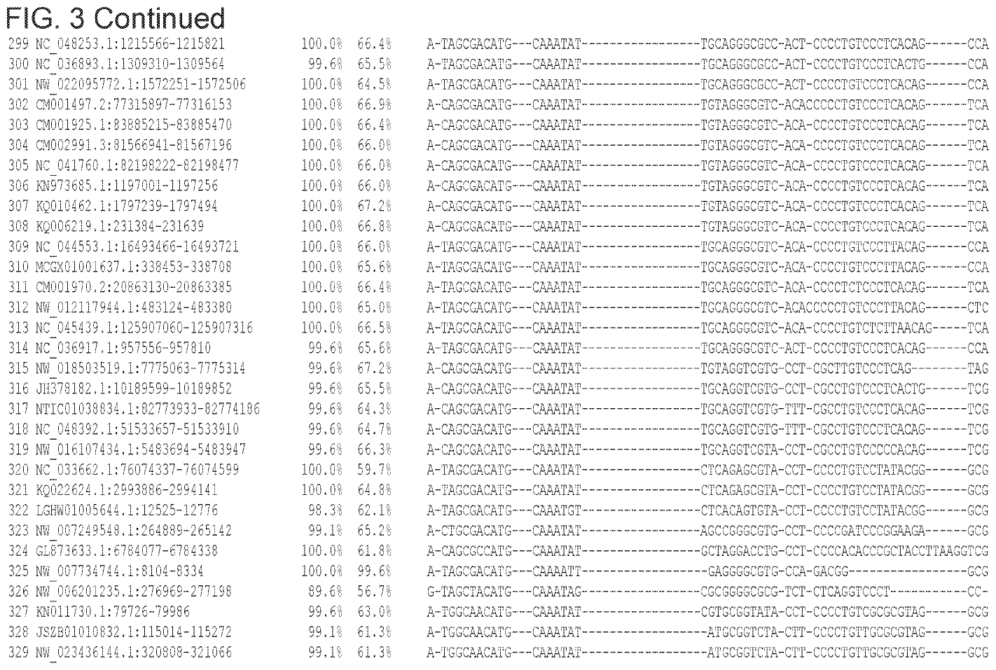

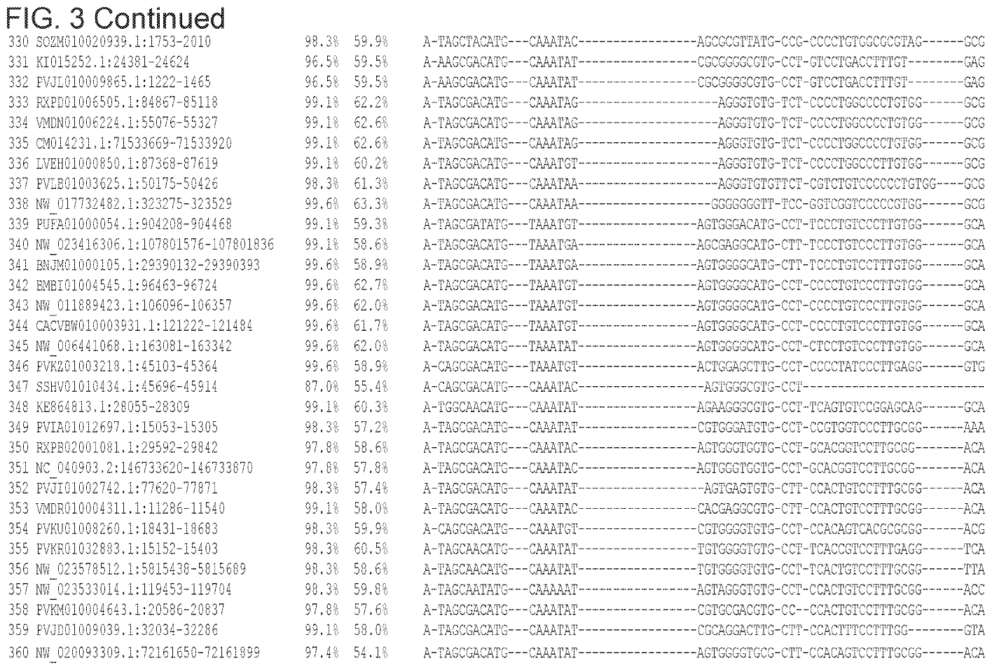

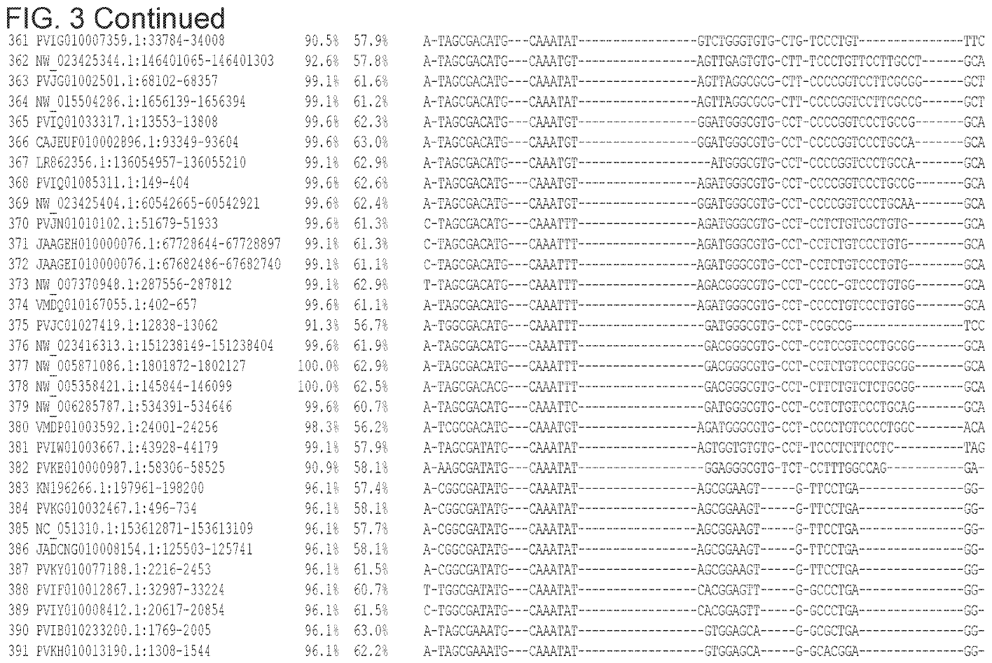

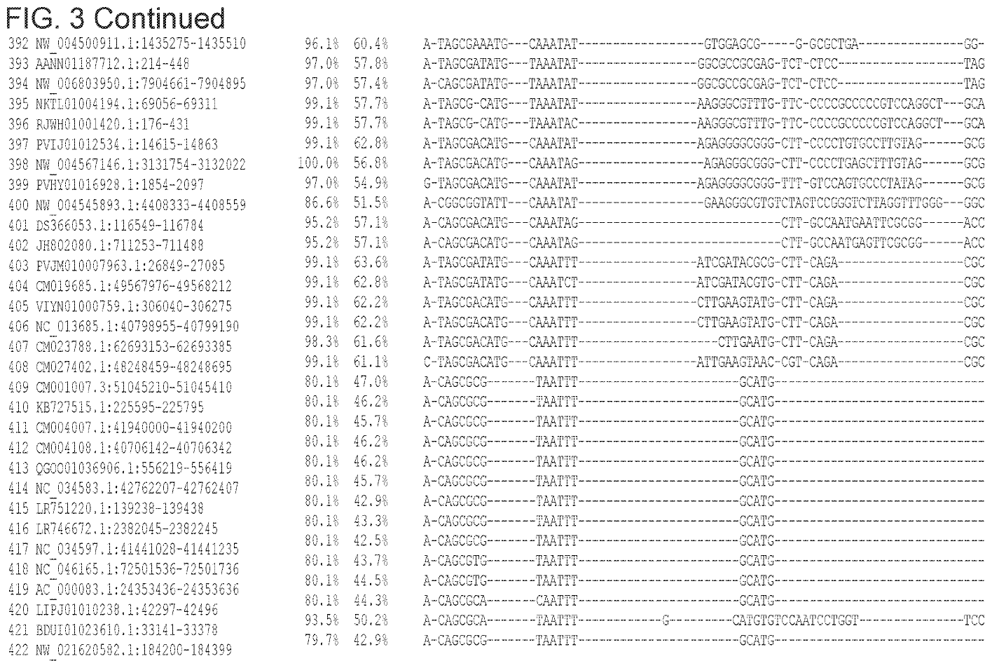

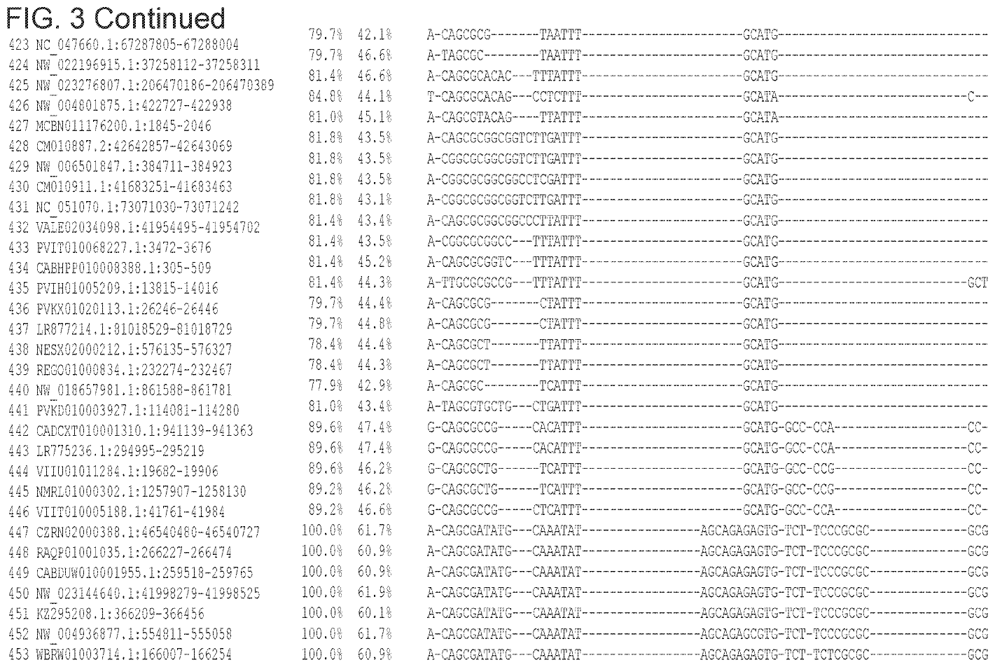

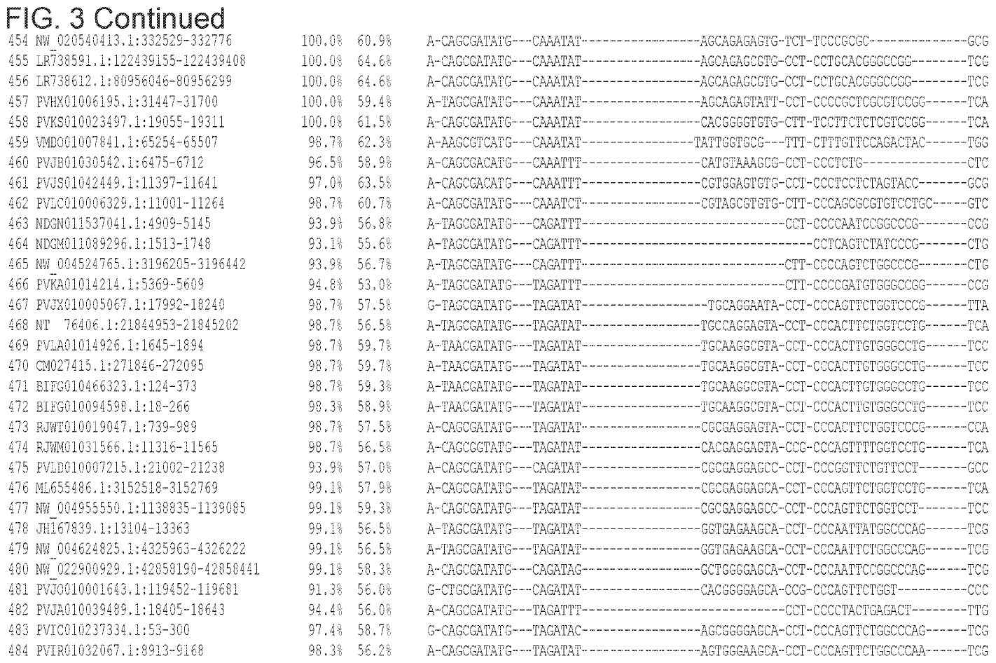

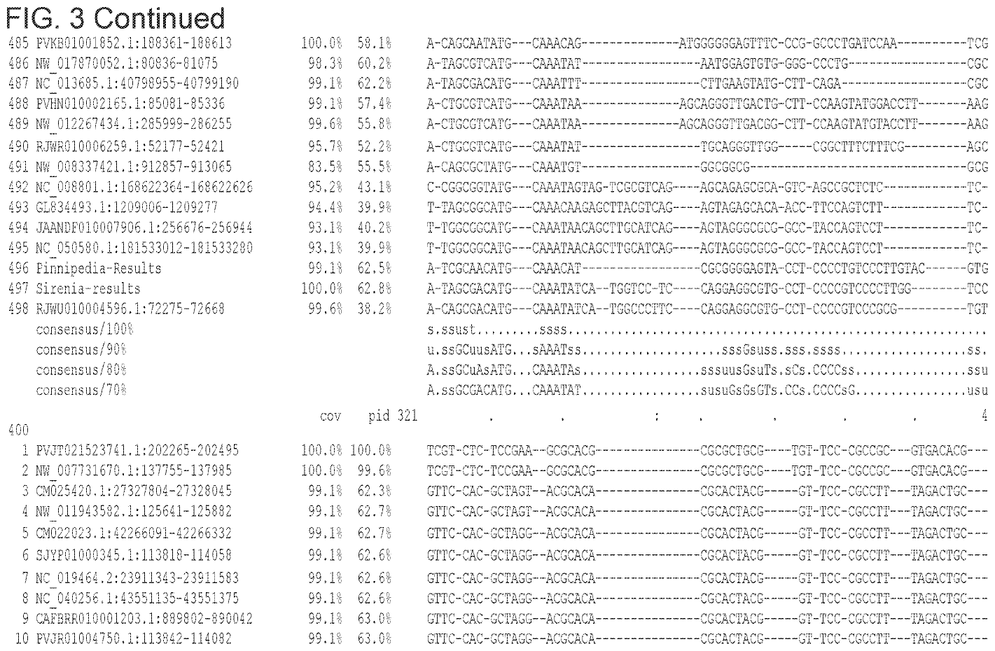

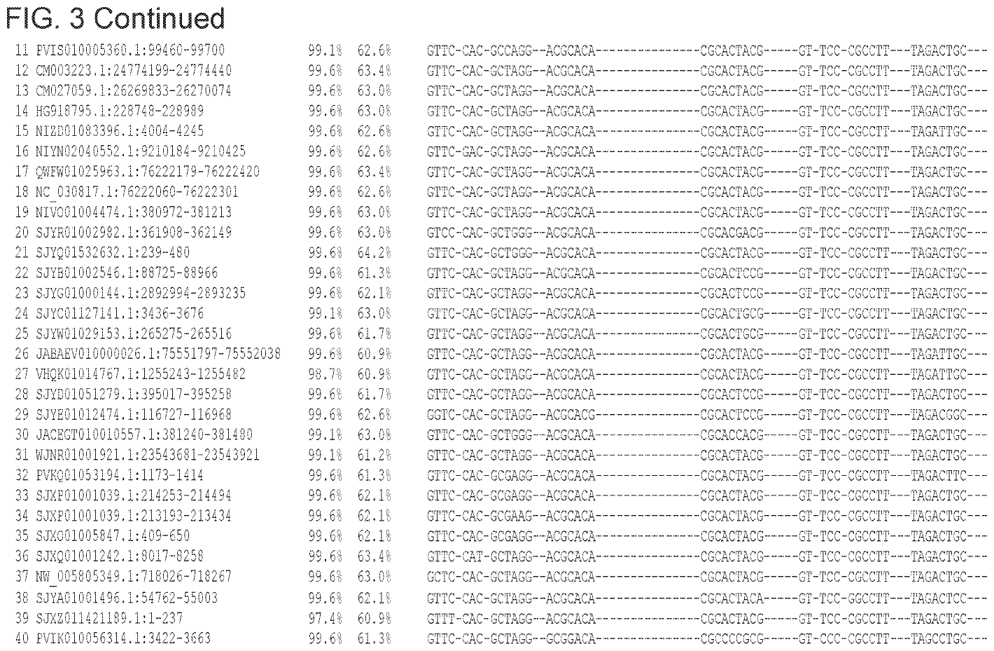

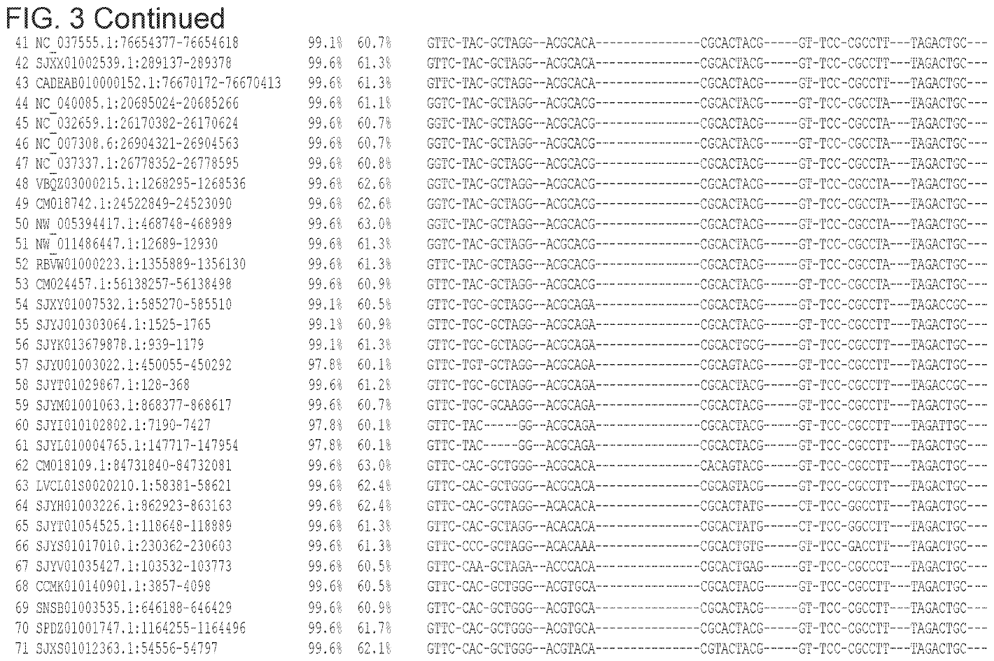

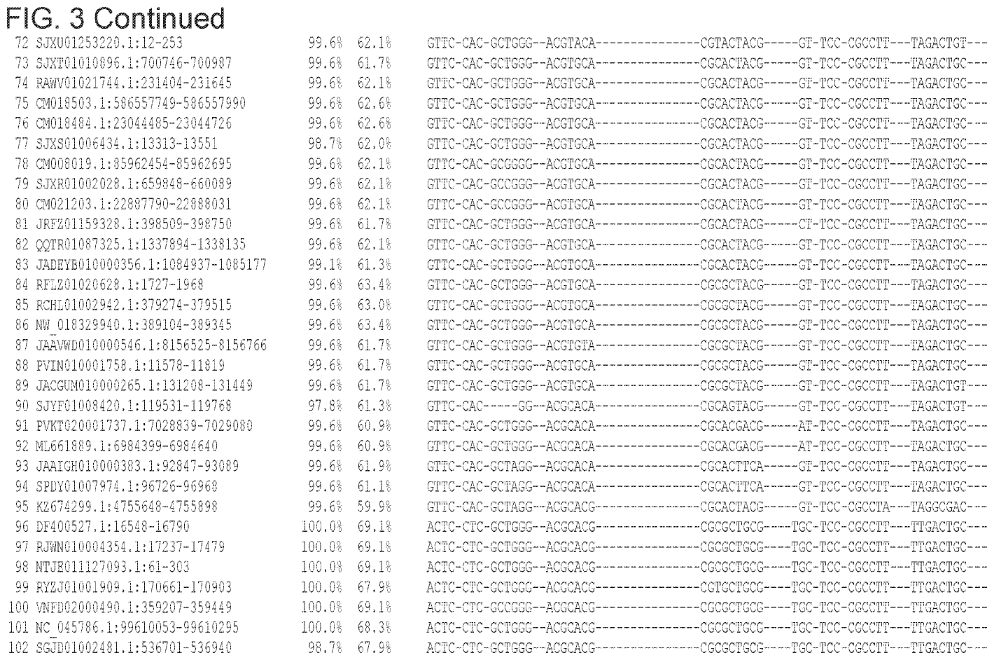

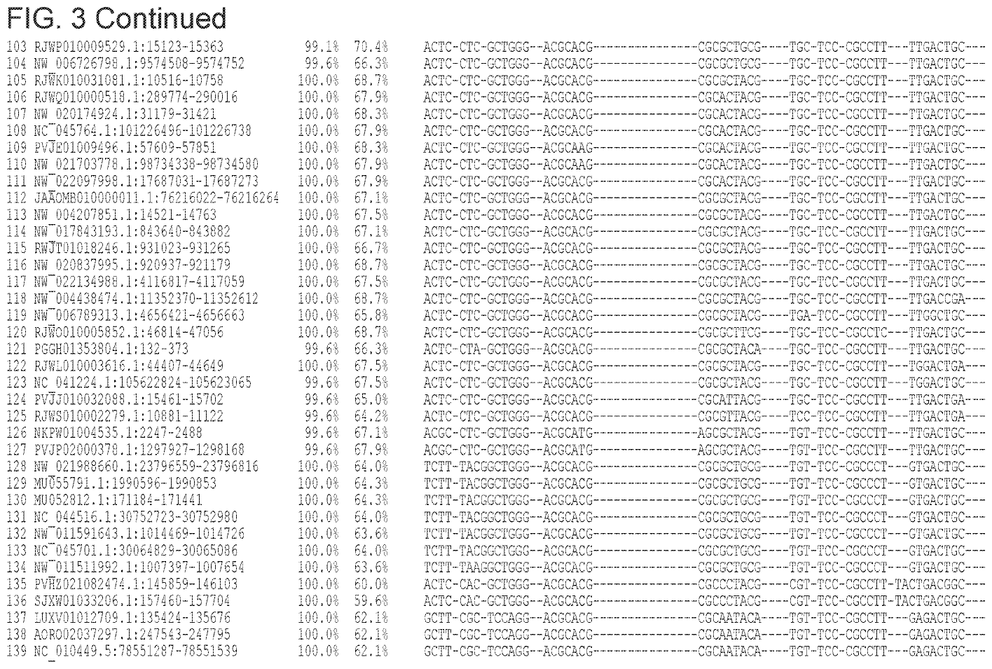

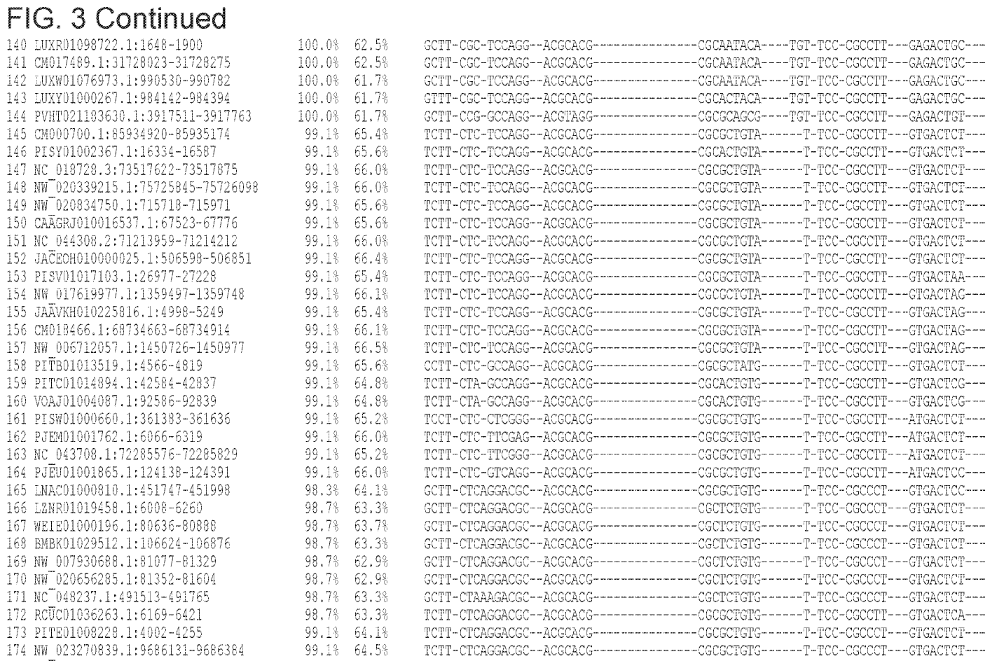

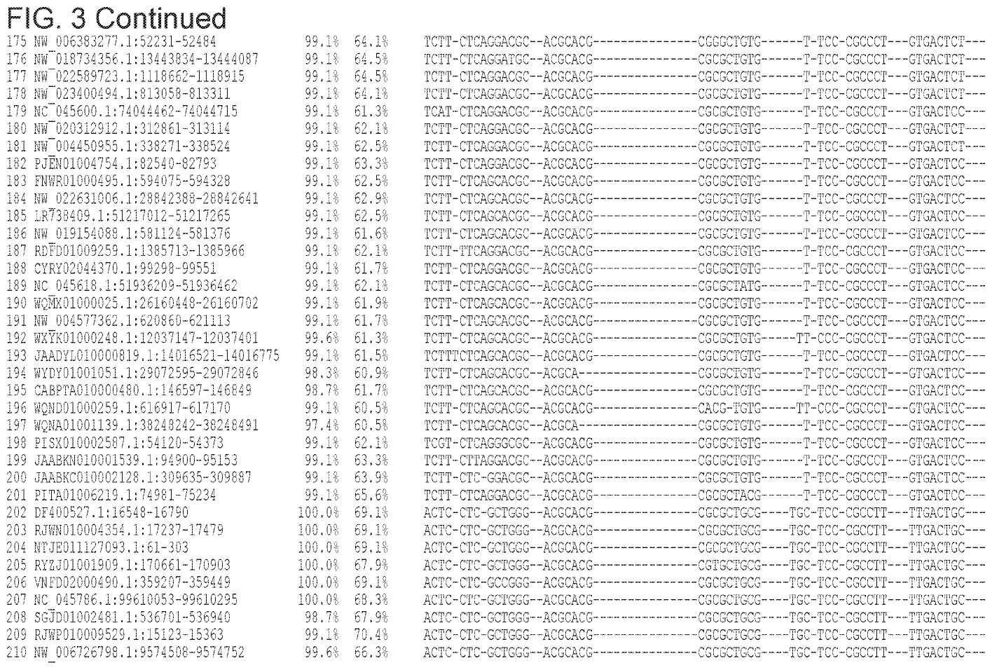

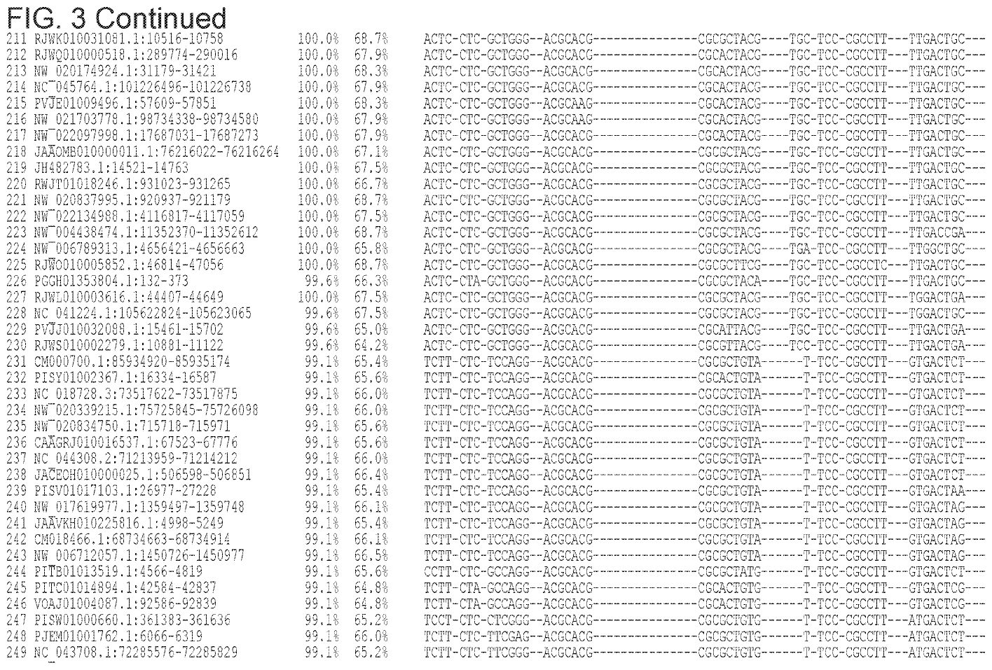

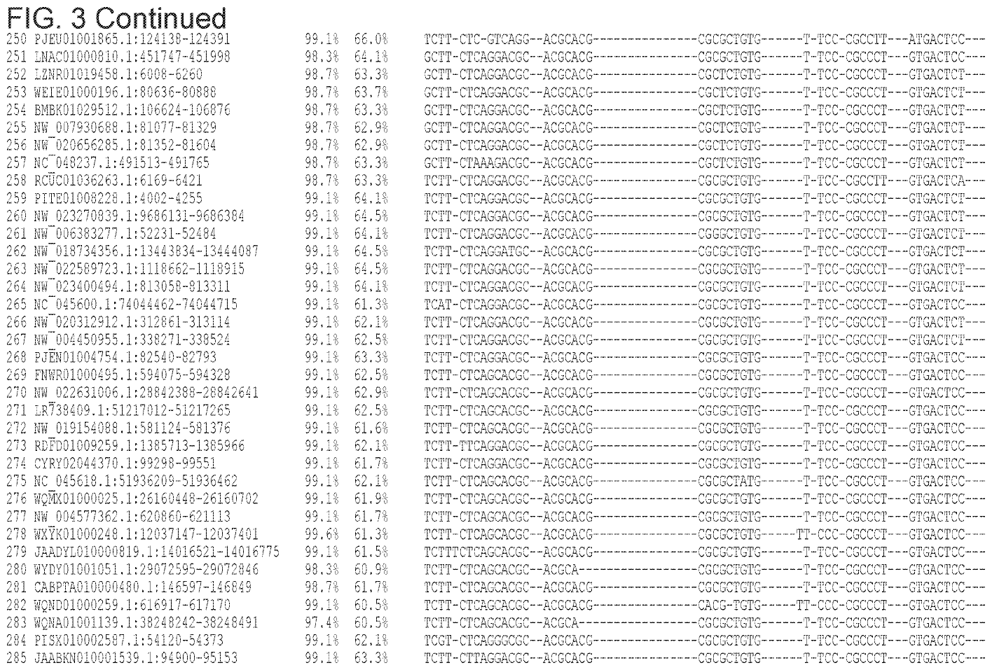

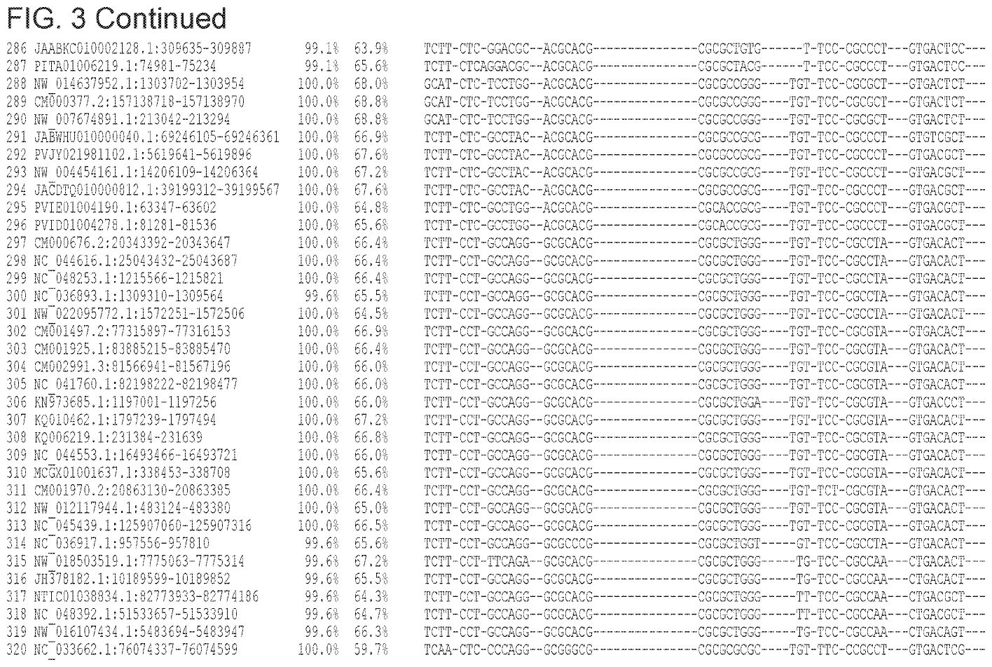

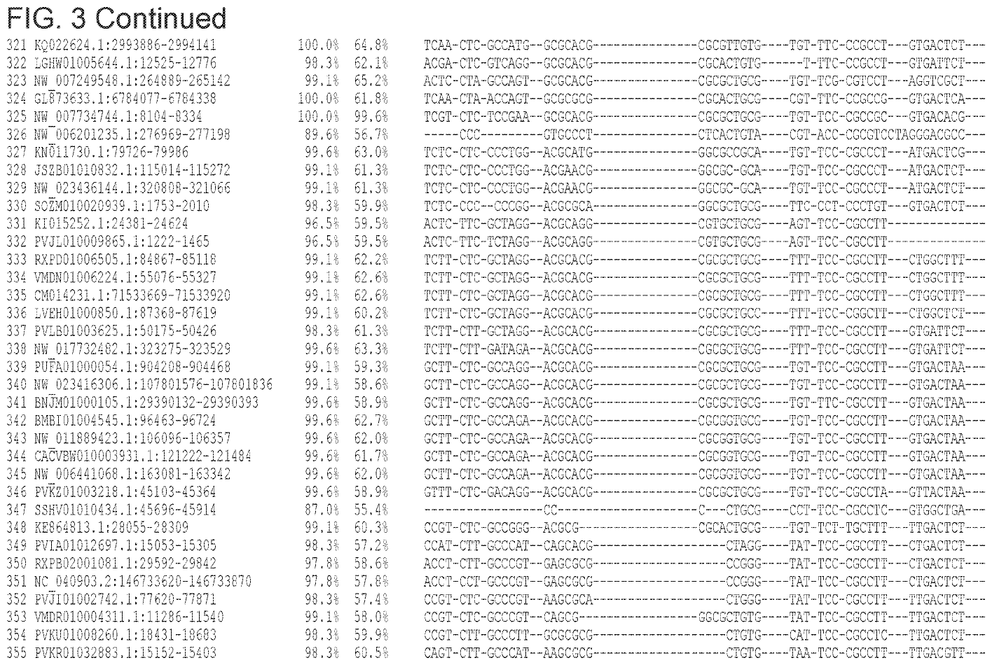

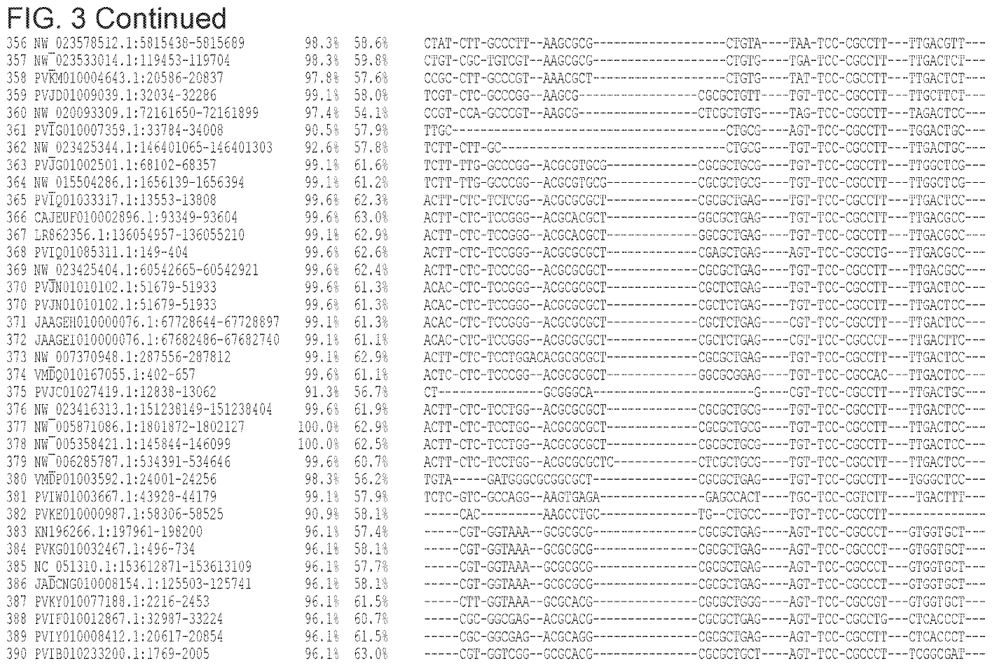

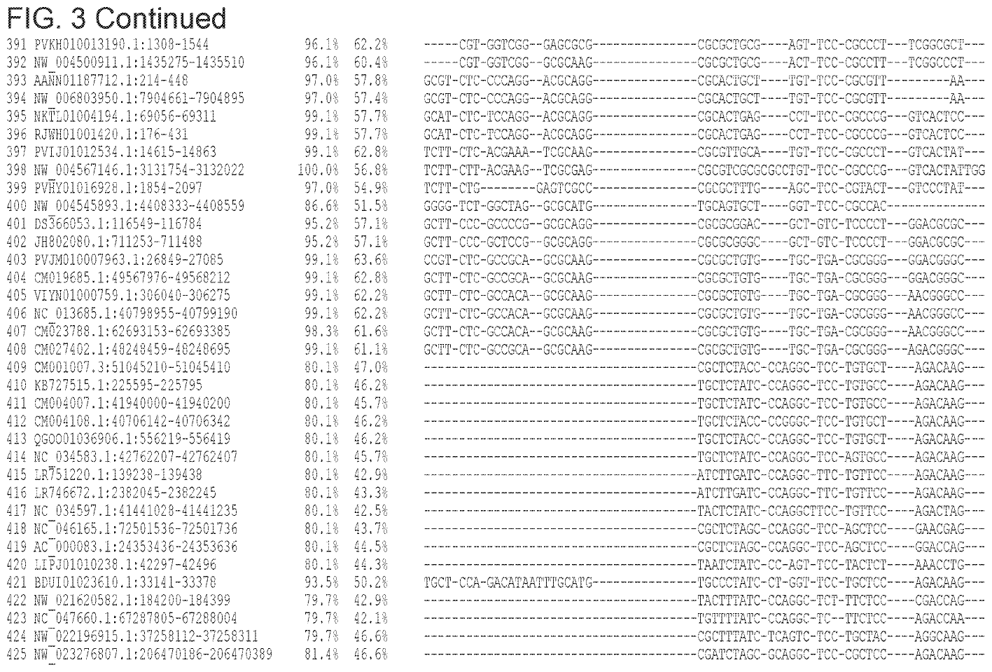

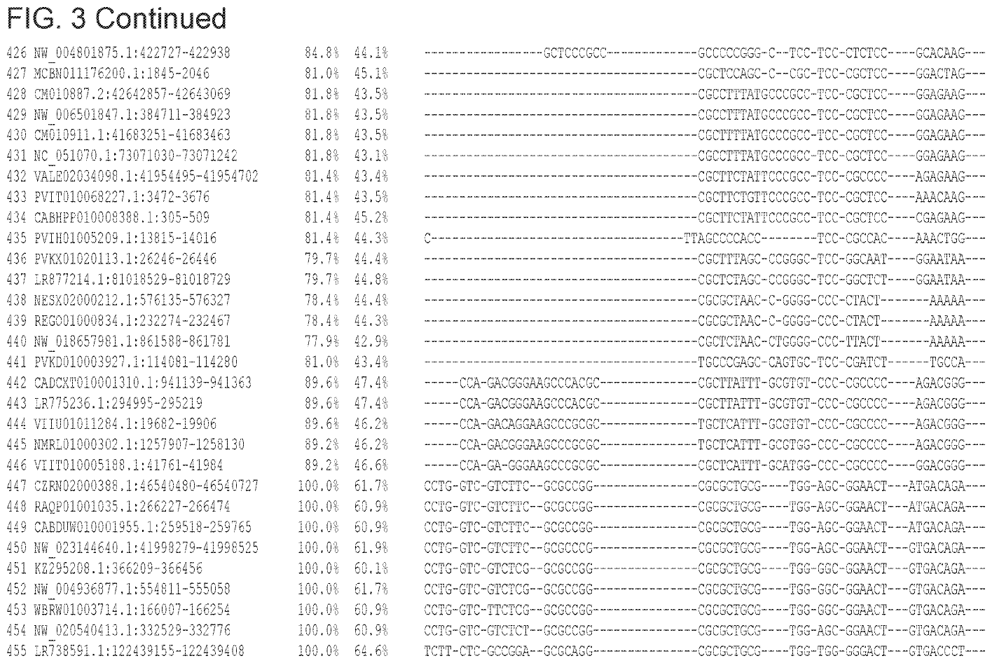

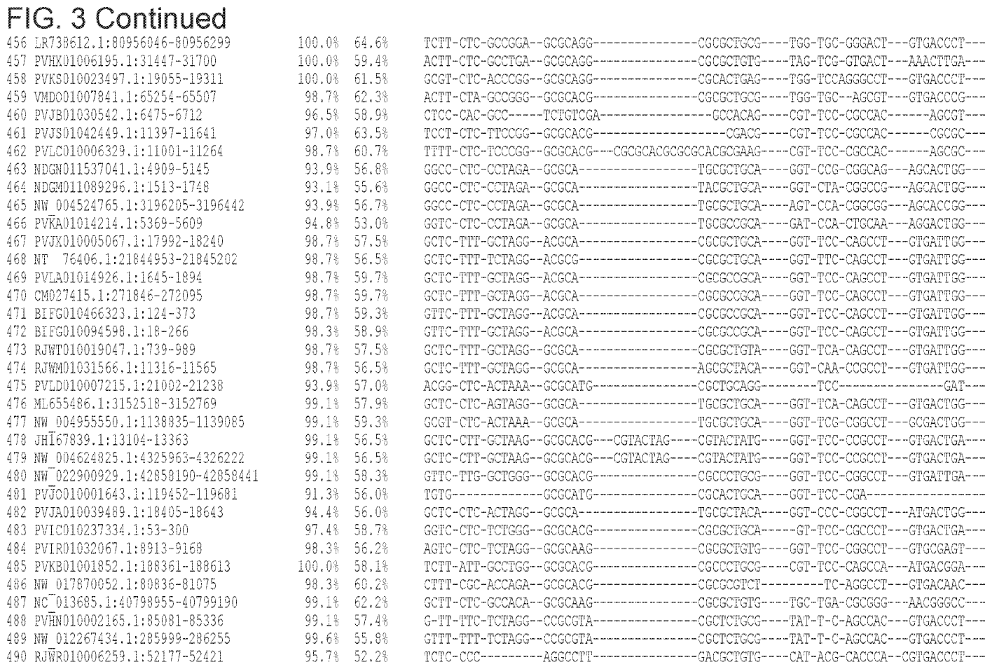

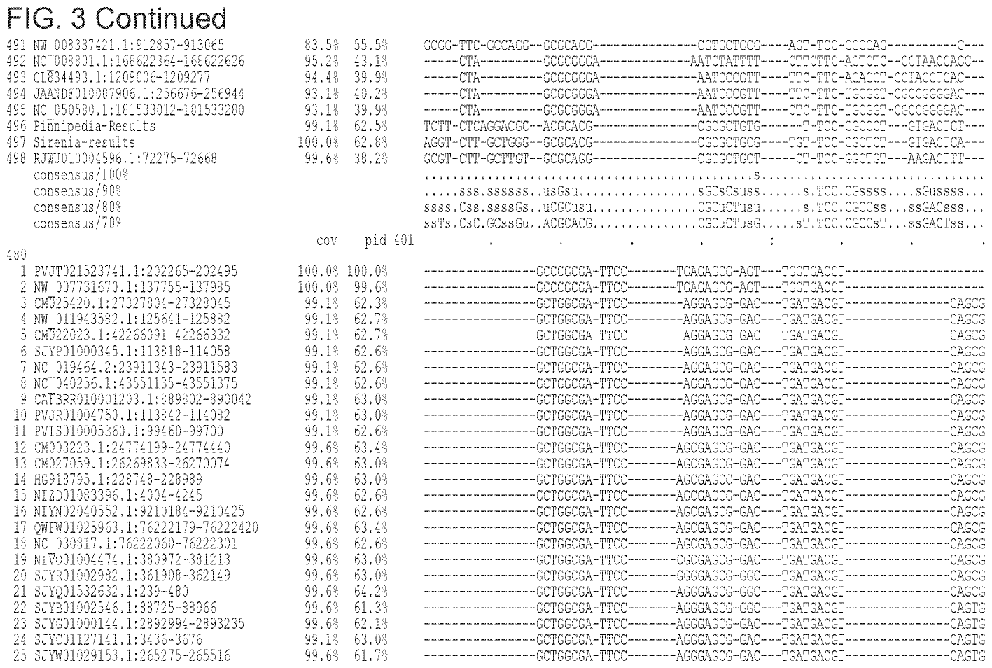

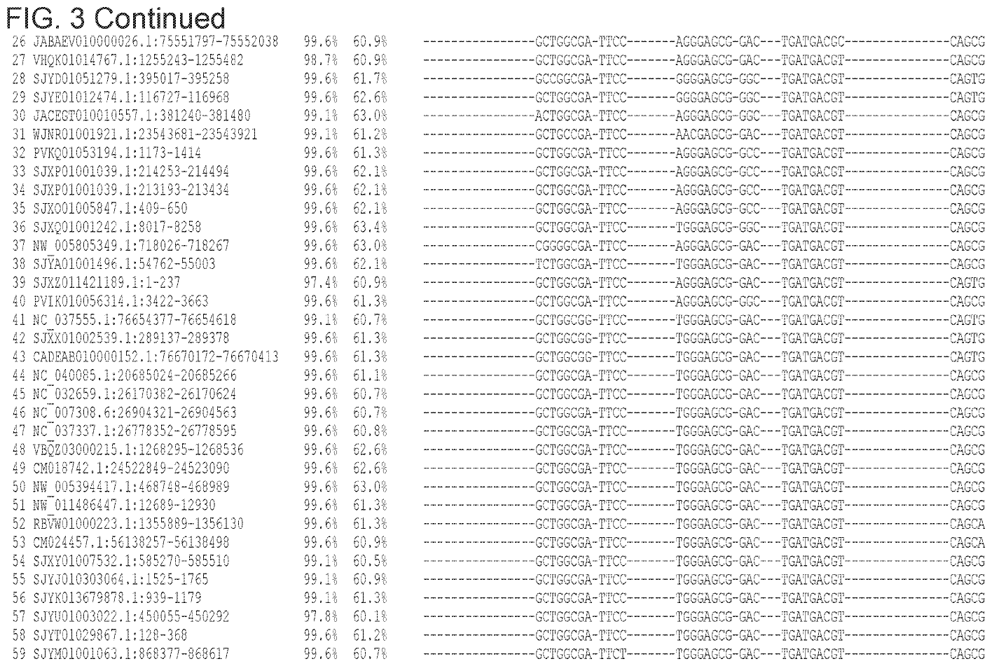

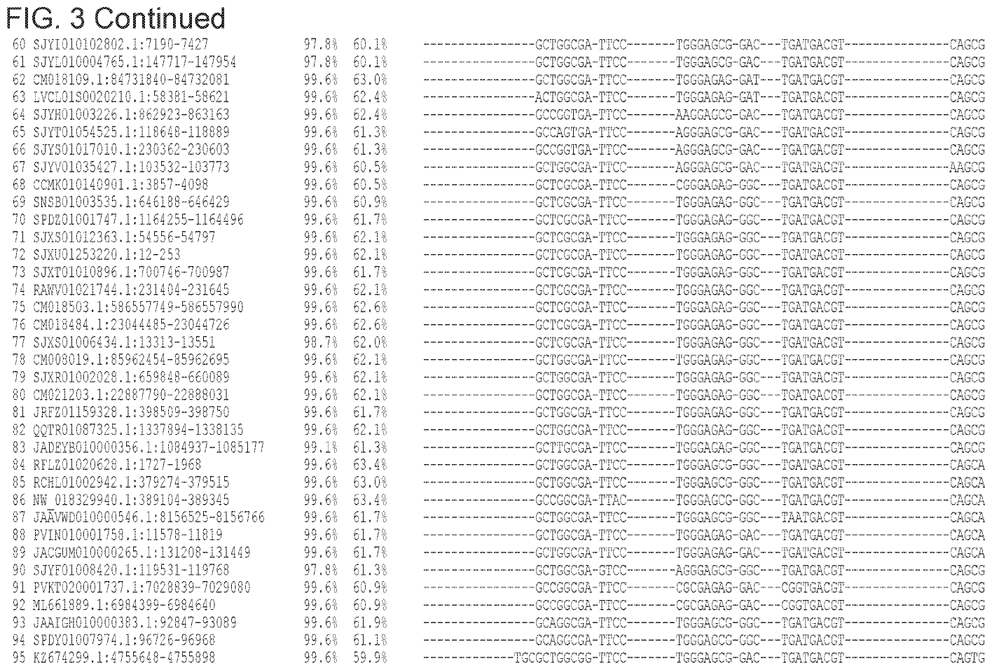

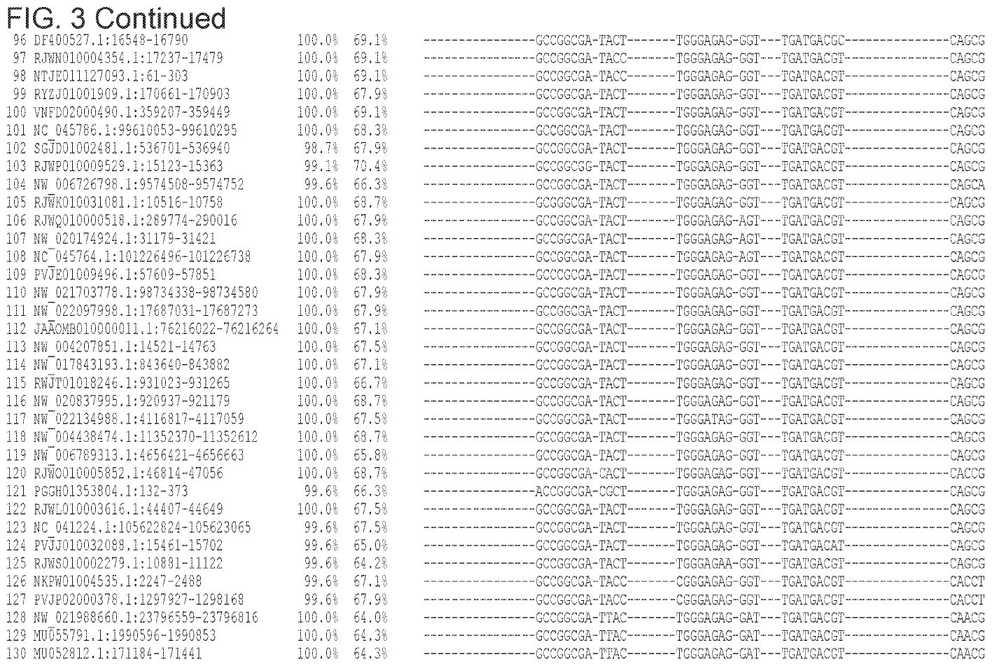

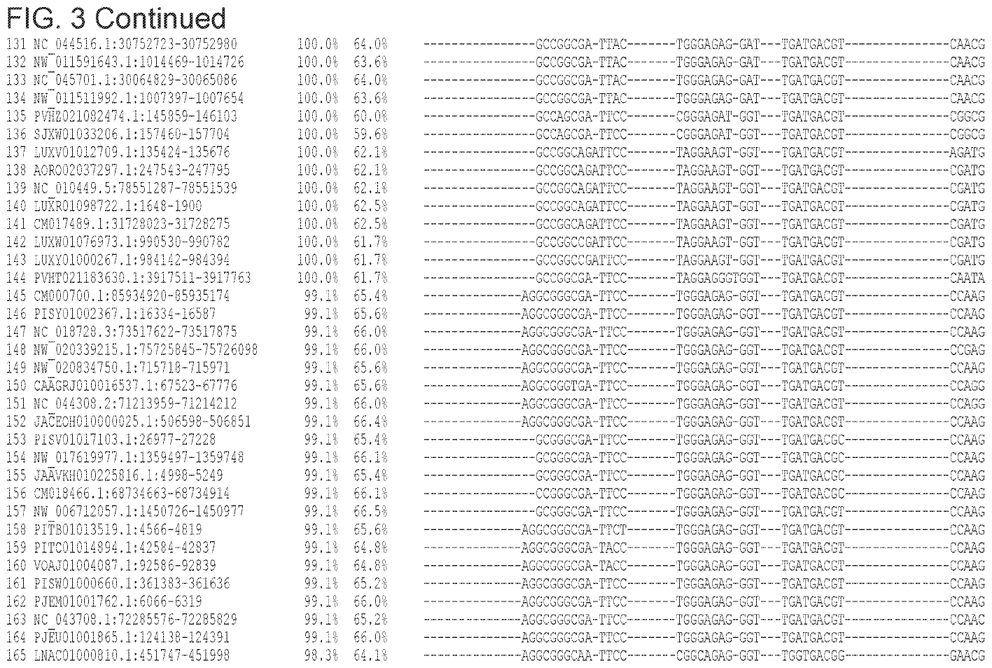

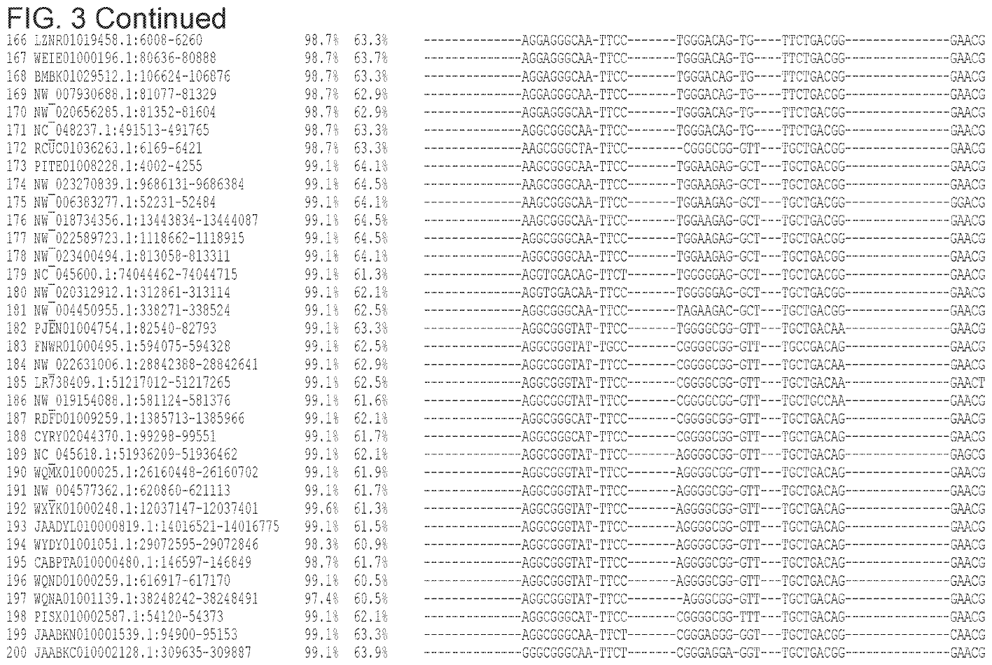

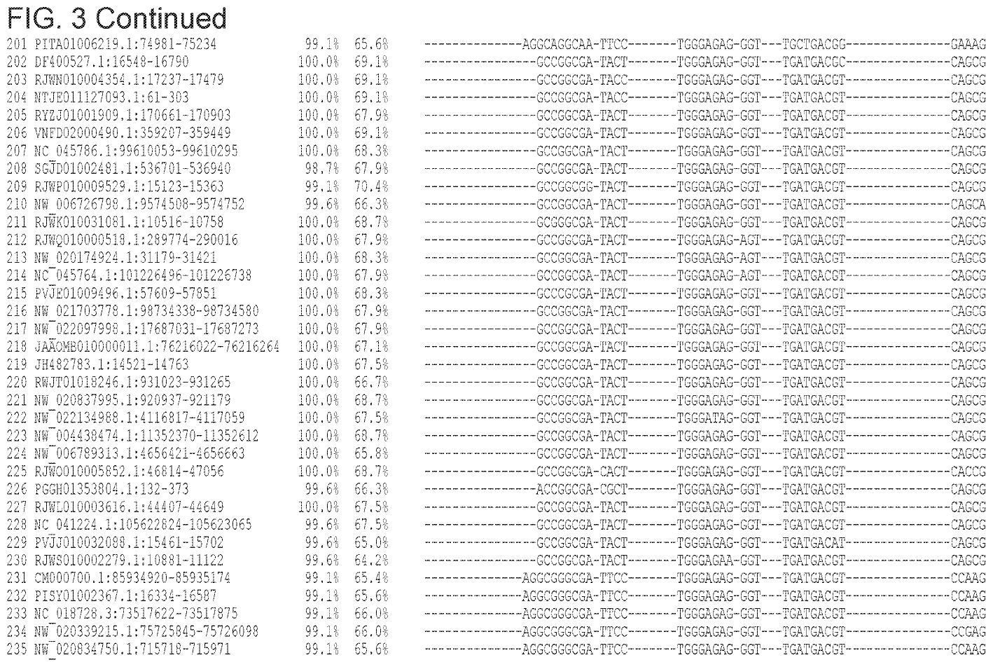

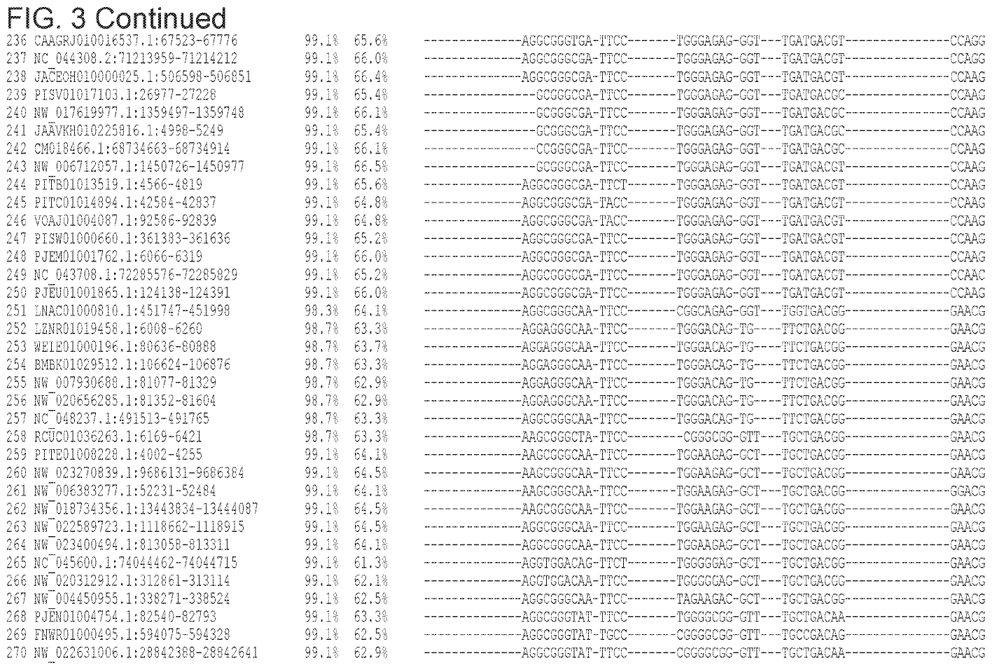

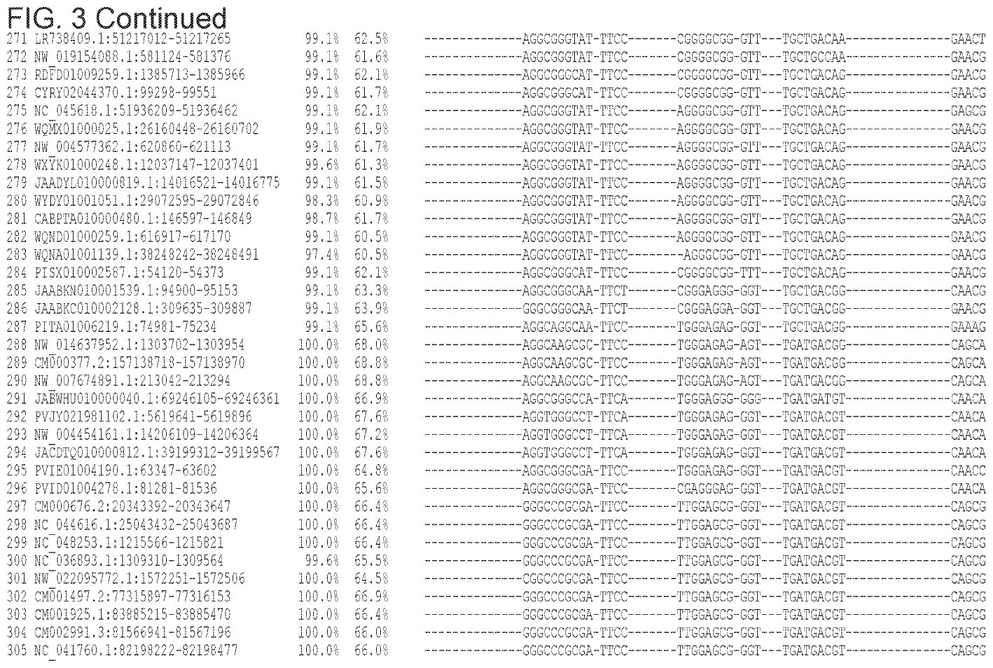

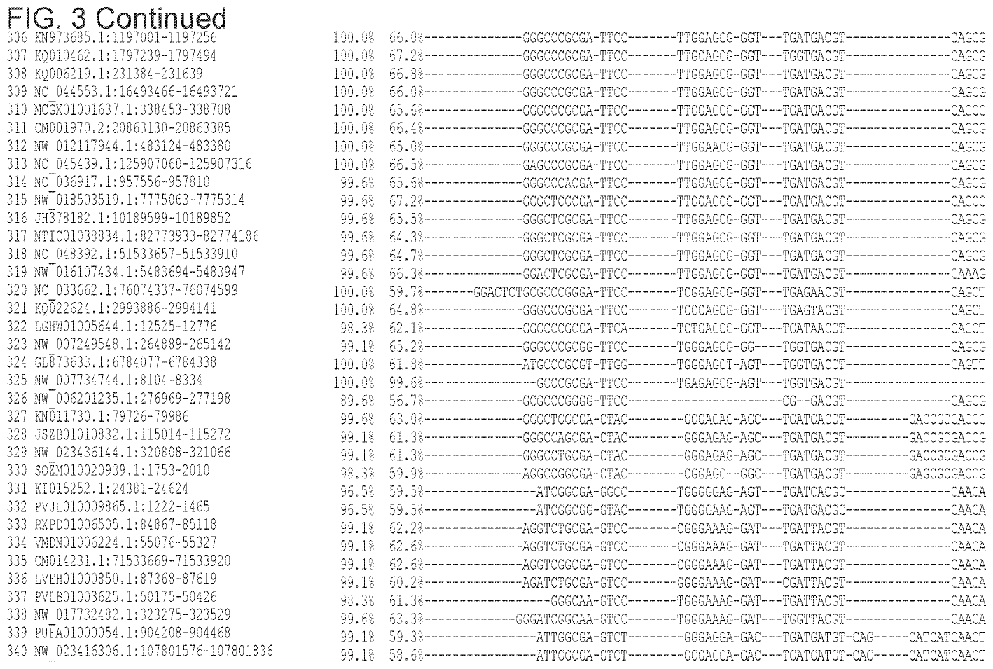

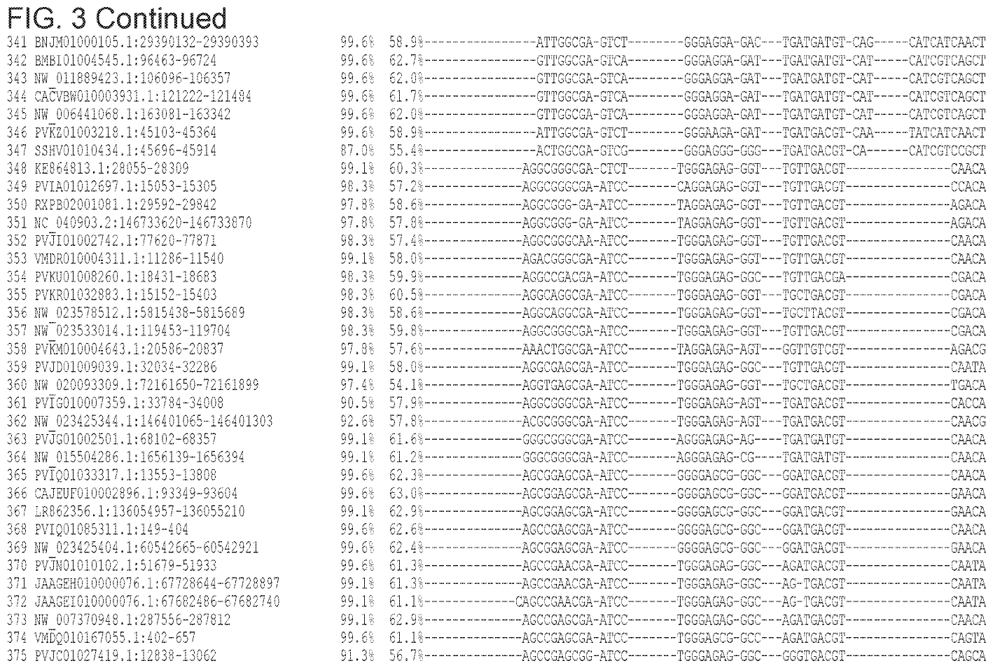

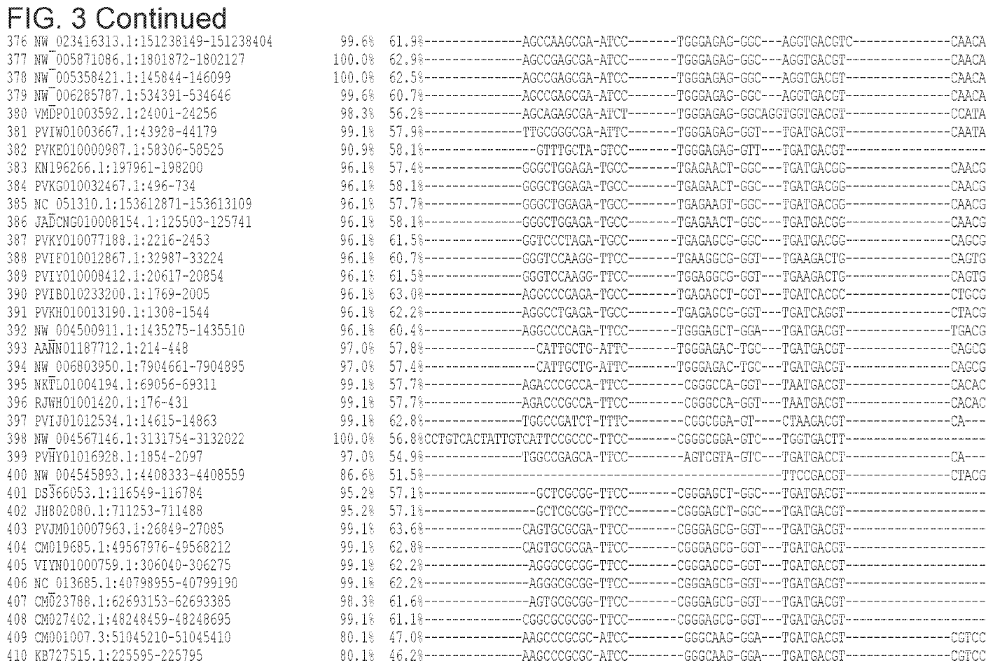

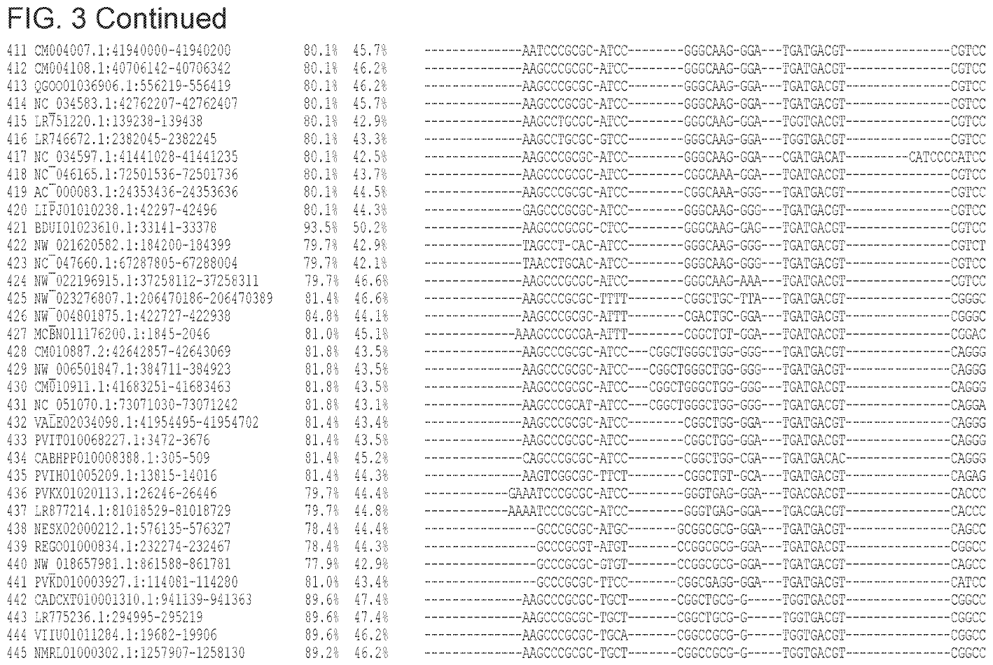

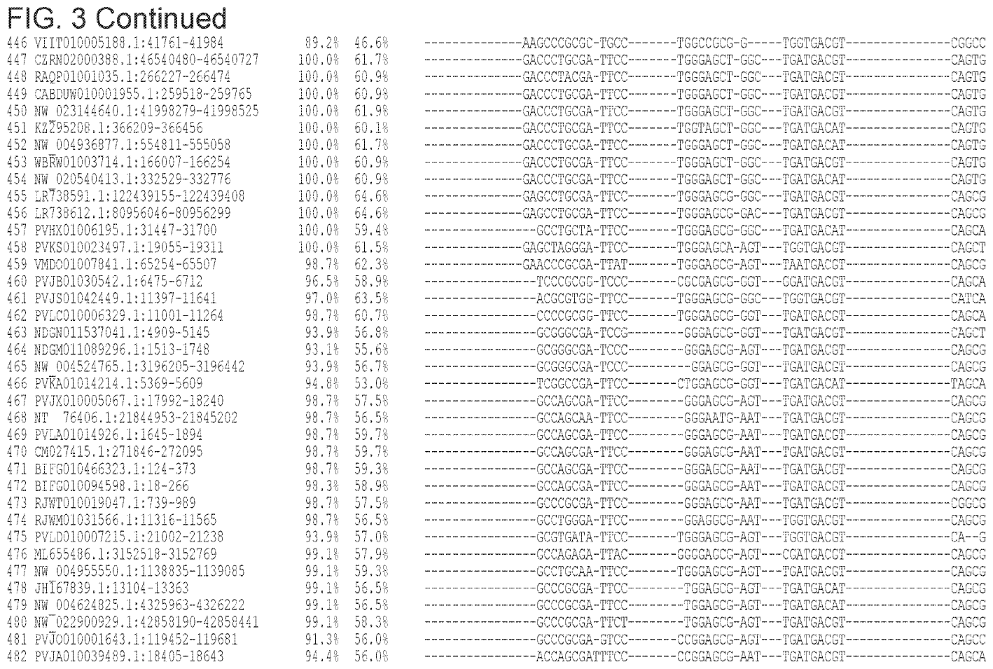

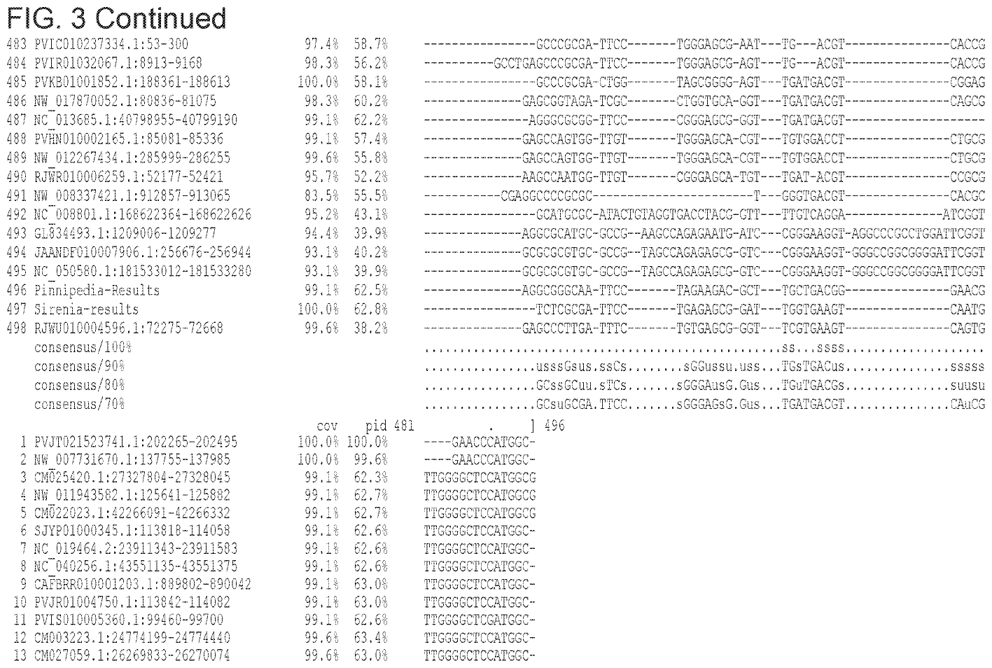

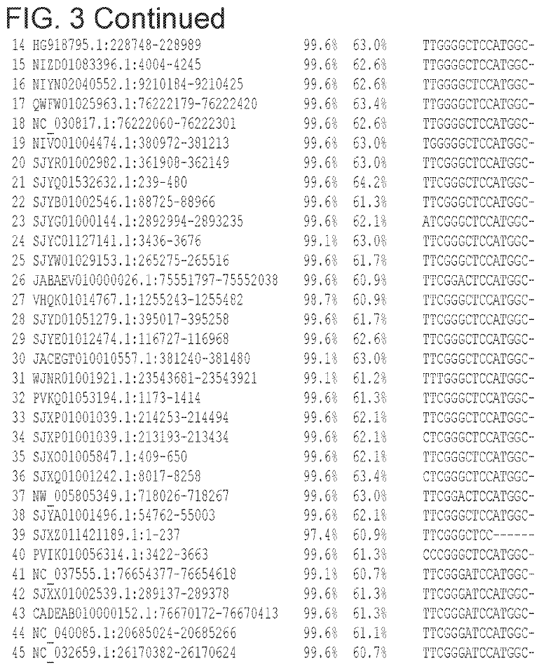

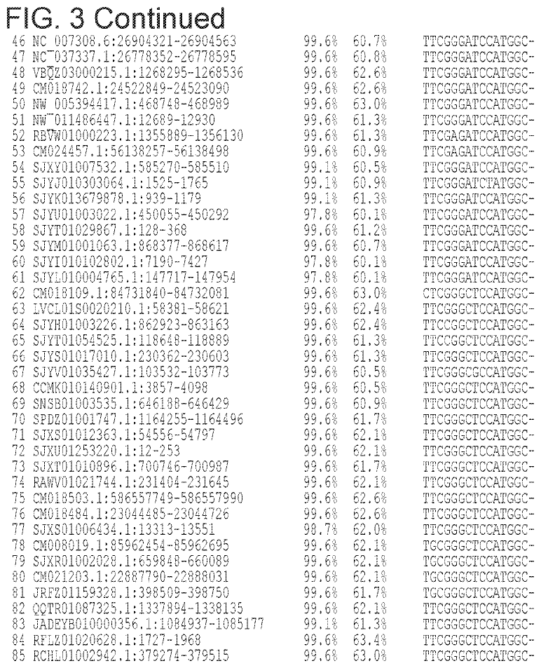

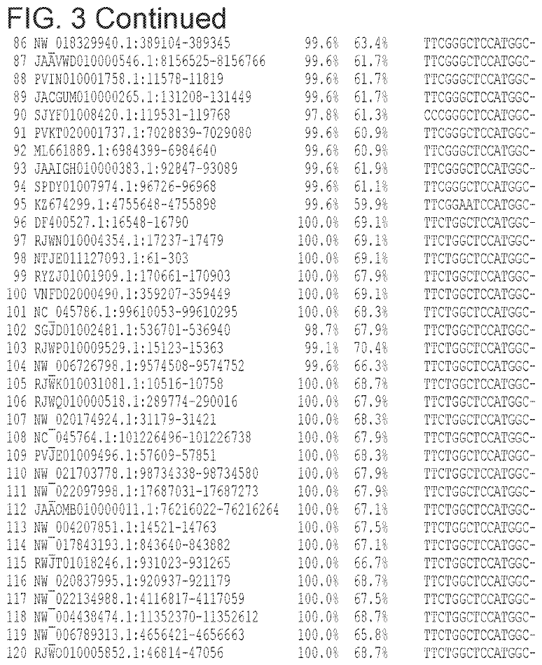

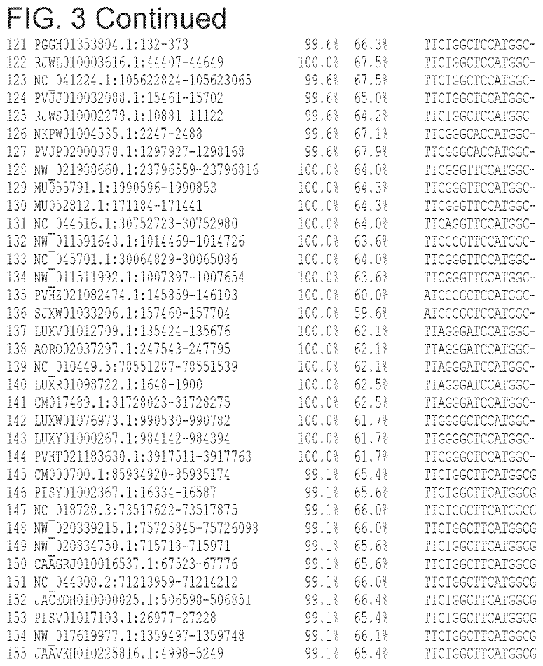

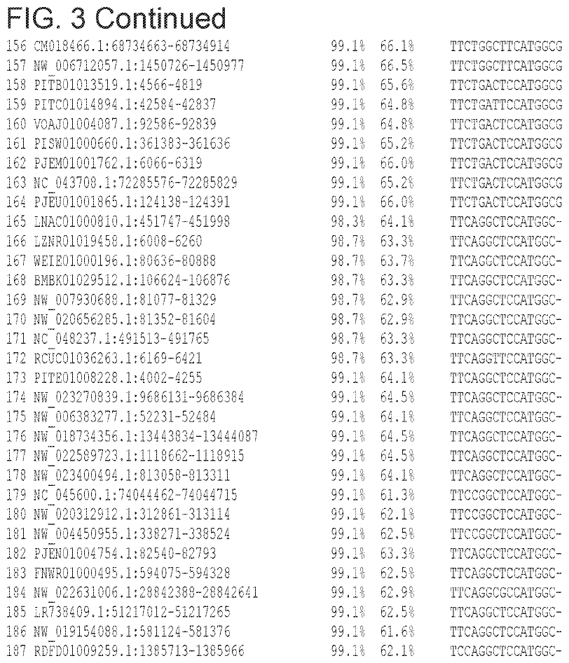

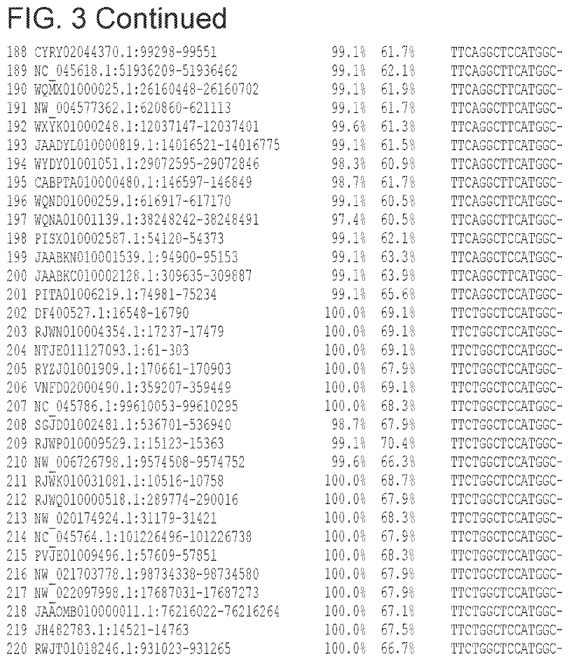

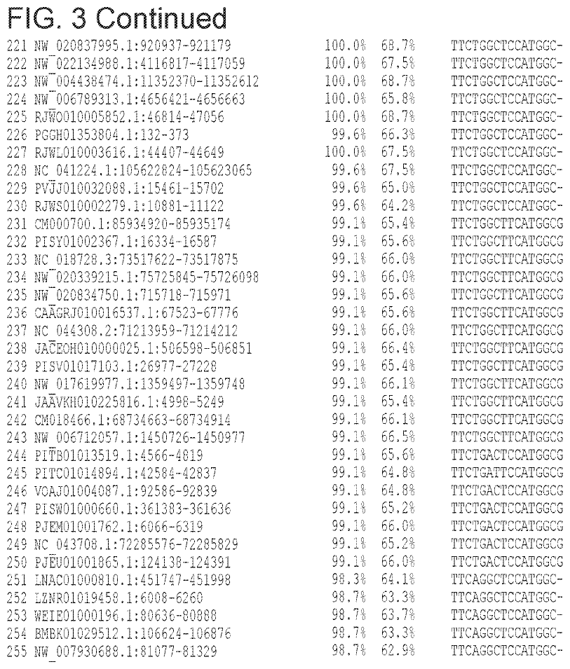

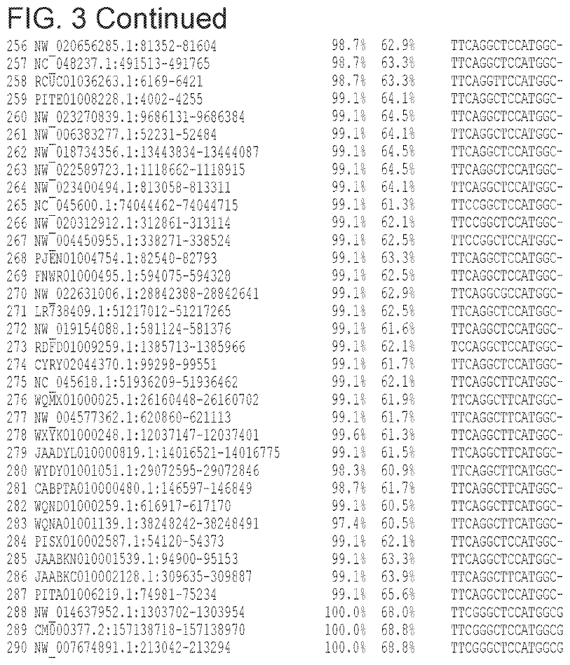

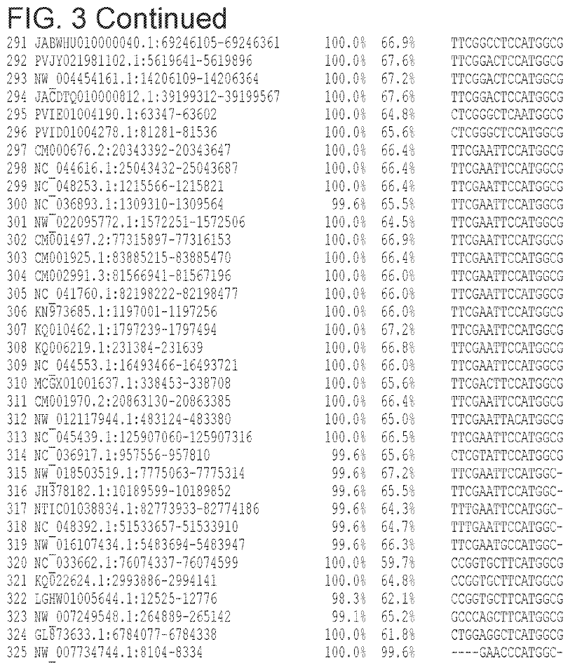

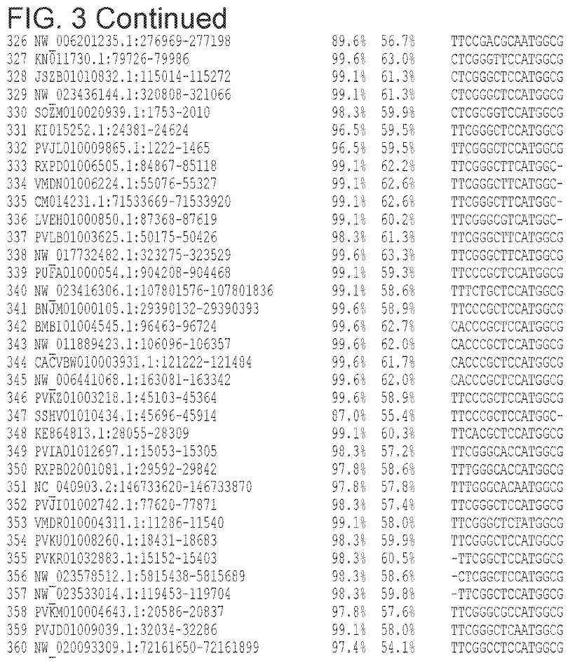

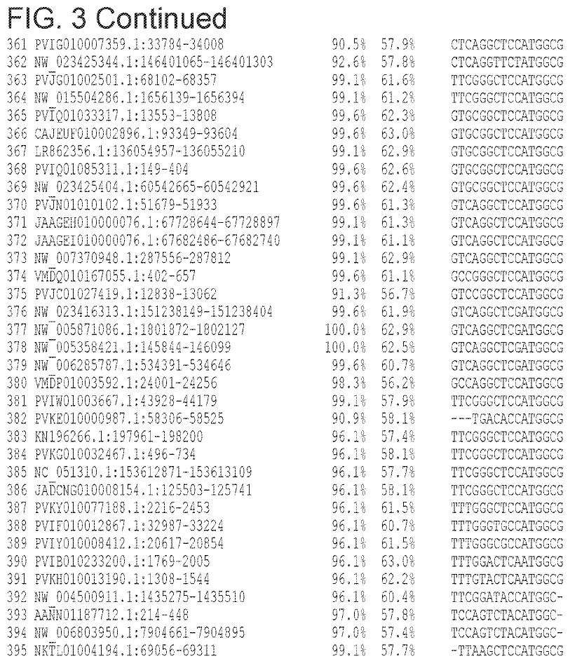

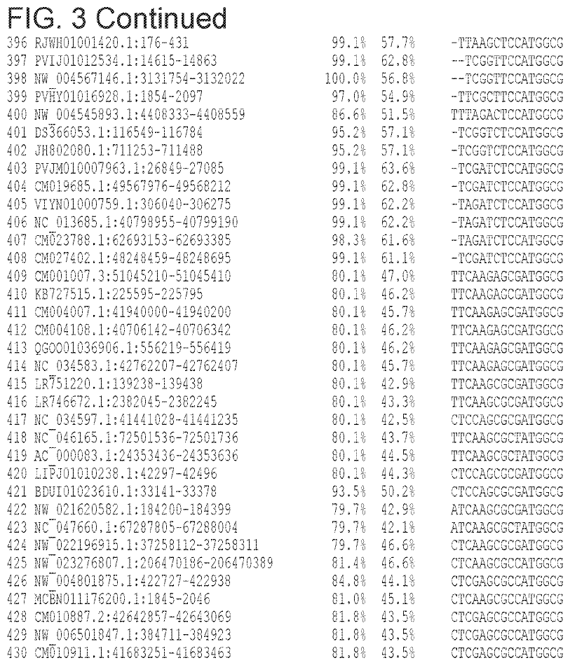

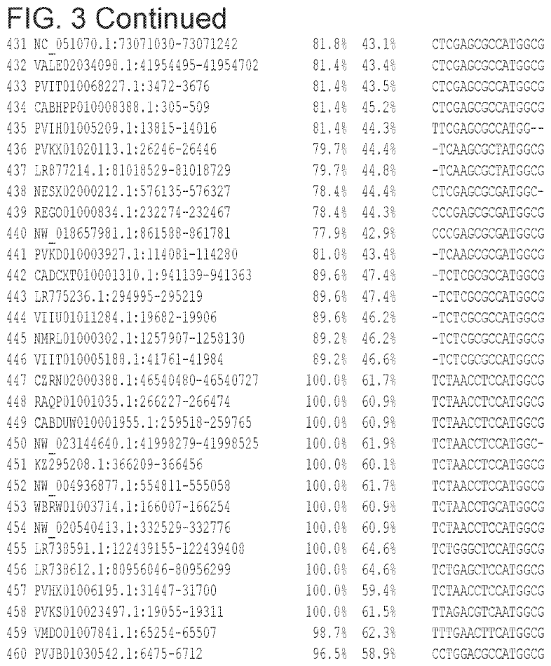

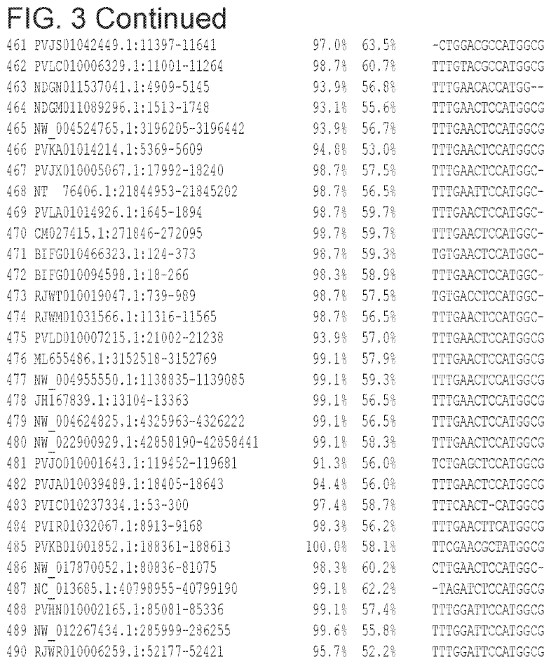

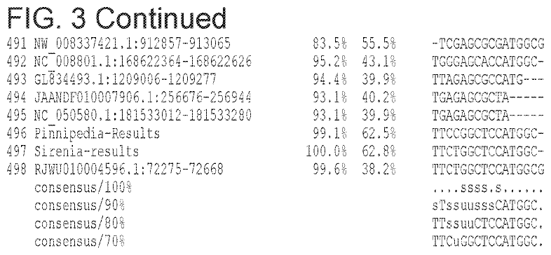

In certain embodiments, the promoter comprises a nucleotide sequence having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a functional fragment or variant (e.g., codon optimized) thereof. In some embodiments, the promoter comprises the nucleotide sequence of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a functional fragment or variant (e.g., codon optimized) thereof.

In certain embodiments, a functional fragment comprises a truncation of from about 10 bases to about 70 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 10 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 20 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 30 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490) as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490) as numbered in FIG. 3)). In certain embodiments, a functional fragment comprises a truncation of about 40 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490) as numbered in FIG. 3)). In certain embodiments, a functional fragment comprises a truncation of about 50 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 60 bases at the 5′ end, the 3′ end, or both the 5′ and 3′ ends of any one of SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)). In certain embodiments, a functional fragment comprises a truncation of about 70 bases at the 5′ end, the 3′ end, or both the 5′ and 3′ ends of any one of S SEQ ID NO: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3) or a variant thereof (e.g., a variant having at least 85%, 90%, 95%, 96%, 97%, 98%, 99%, or 100% identity to any one of SEQ ID NO: 107-255 or the portion of any one of SEQ ID NOS: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3)).

In certain embodiments, the functional fragment comprise at least a transcription factor binding site. Identification of transcription factor binding sites can be determined by consensus, or by using a differential distance matrix or multidimensional scaling (De Bleser P. et al. (2007) GENOME BIOL 8(5):R83. In certain embodiments, a functional fragment comprises at least a transcription factor binding sites selected from Staf, DSE, PSE, c-REL, GATA-1, GATA-2, and CREB. A functional fragment can comprise the B recognition sequence (BRE) or TATA box.

In certain embodiments, the promoter comprises a TATA mutation. In certain embodiments, the TATA mutation is a TATAA→TCGAA mutation.

In certain embodiments, the promoter is not one or more of an alpaca H1 promoter (SEQ ID NO: 70), an armadillo H1 promoter (SEQ ID NO: 71), a baboon H1 promoter (SEQ ID NO: 72), a bottlenose dolphin H1 promoter (SEQ ID NO: 73), a bushbaby H1 promoter (SEQ ID NO: 74), a cat H1 promoter (SEQ ID NO: 75), a chimp H1 promoter (SEQ ID NO: 76), a cow H1 promoter (SEQ ID NO: 77), a crab-eating macaque H1 promoter (SEQ ID NO: 78), a dog H1 promoter (SEQ ID NO: 79), an elephant H1 promoter (SEQ ID NO: 80), a European hedgehog H1 promoter (SEQ ID NO: 81), a ferret H1 promoter (SEQ ID NO: 82), a gorilla H1 promoter (SEQ ID NO: 83), a green monkey H1 promoter (SEQ ID NO: 84), a guinea pig H1 promoter (SEQ ID NO: 85), a horse H1 promoter (SEQ ID NO: 86), a human H1 promoter (SEQ ID NO: 87), a kangaroo rat H1 promoter (SEQ ID NO: 88), a large flying fox H1 promoter (SEQ ID NO: 89), a little brown bat H1 promoter (SEQ ID NO: 90), a marmoset H1 promoter (SEQ ID NO: 91), a mouse H1 promoter (SEQ ID NO: 92 or SEQ ID NO: 93), a northern treeshrew H1 promoter (SEQ ID NO: 94), an orangutan H1 promoter (SEQ ID NO: 95), a panda H1 promoter (SEQ ID NO: 96), a pig H1 promoter (SEQ ID NO: 97), a pika H1 promoter (SEQ ID NO: 98), a rabbit H1 promoter (SEQ ID NO: 99), a rat H1 promoter (SEQ ID NO: 100), a rock hyax H1 promoter (SEQ ID NO: 101), a sheep H1 promoter (SEQ ID NO: 102), a squirrel H1 promoter (SEQ ID NO: 103), a tarsier H1 promoter (SEQ ID NO: 104), a two-toed sloth H1 promoter (SEQ ID NO: 105), or a white cheeked gibbon H1 promoter (SEQ ID NO: 106). In certain embodiments, the promoter is not one or more of an SRP-RPS29 promoter (SEQ ID NO: 241), a 7sk1 promoter (SEQ ID NO: 242), a 7sk2 promoter (SEQ ID NO: 243), a 7sk3 promoter (SEQ ID NO: 244), an RMRP-CCDC107 promoter (SEQ ID NO: 245), an SRP-ALOXE3 promoter (SEQ ID NO: 246), a CGB1 promoter (SEQ ID NO: 247), a CGB2 promoter (SEQ ID NO: 248), a Med16-1 promoter (SEQ ID NO: 249), a Med16-2 promoter (SEQ ID NO: 250), a DPP9-1 promoter (SEQ ID NO: 251), a DPP9-2 promoter (SEQ ID NO: 252), a DPP93 promoter (SEQ ID NO: 253), a SNORD13-C8orf41 promoter (SEQ ID NO: 254), and a THEM259 promoter (SEQ ID NO: 255).

In certain embodiments, a nucleic acid comprising a promoter described herein further comprises a 5′UTR including at least a portion of a beta-globin 5′UTR sequence or a Kozak sequence. In certain embodiments, the 5′UTR includes the nucleotide sequence 5″-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a promoter described herein further comprises a terminator sequence. In certain embodiments, the terminator sequence comprises one of the terminator sequences in TABLE 2.

TABLE 2

| | |
|---|---|
| a synthetic | AATAAAATATCTTTATTTTCATTAC |
| poly(A) | ATCTGTGTGTTGGTTTTTT |
| sequence (SPA) | GTGTG (SEQ ID NO: 258) |
| SPA and Pause | AATAAAATATCTTTATTTTCATTAC |
| | ATCTGTGTGTTGGTTTTTTGTGTGA |
| | ATCGATAGTACTAACATACGCTCTC |
| | CATCAAAACAAAACGAAACAAAACA |
| | AACTAGCAAAATAGGCTGTCCCCAG |
| | TGCAAGTGCAGGTGCCAGAACATTT |
| | CTCT (SEQ ID NO: 259); |
| SV40 (240 bp) | ATCTAGATAACTGATCATAATCAGC |
| | CATACCACATTTGTAGAGGTTTTAC |
| | TTGCTTTAAAAAACCTCCCACACCT |
| | CCCCCTGAACCTGAAACATAAAATG |
| | AATGCAATTGTTGTTGTTAACTTGT |
| | TTATTGCAGCTTATAATGGTTACAA |
| | ATAAAGCAATAGCATCACAAATTTC |
| | ACAAATAAAGCATTTTTTTCACTGC |
| | ATTCTAGTTGTGGTTTGTCCAAACT |
| | CATCAATGTATCTTA |
| | (SEQ ID NO: 260) |
| SV 40-mini | TTGTTTATTGCAGCTTATAATGGTT |
| (120 bp) | ACAAATAAAGCAATAGCATCACAAA |
| | TTTCACAAATAAAGCATTTTTTTCA |
| | CTGCATTCTAGTTGTGGTTTGTCCA |
| | AACTCATCAATGTATCTTAT |
| | (SEQ ID NO: 261) |
| bGH poly A | CGACTGTGCCTTCTAGTTGCCAGCC |
| | ATCTGTTGTTTGCCCCTCCCCCGTG |
| | CCTTCCTTGACCCTGGAAGGTGCCA |
| | CTCCCACTGTCCTTTCCTAATAAAA |
| | TGAGGAAATTGCATCGCATTGTCTG |
| | AGTAGGTGTCATTCTATTCTGGGGG |
| | GTGGGGTGGGGCAGGACAGCAAGGG |
| | GGAGGATTGGGAAGACAATAGCAGG |
| | CATGCTGGGGATGCGGTGGGCTCTA |
| | TGG (SEQ ID NO: 262) |
| TKpoly A | GGGGGAGGCTAACTGAAACACGGAA |
| | GGAGACAATACCGGAAGGAACCCGC |
| | GCTATGACGGCAATAAAAAGACAGA |
| | ATAAAACGCACGGGTGTTGGGTCGT |
| | TTGTTCATAAACGCGGGGTTCGGTC |
| | CCAGGGCTGGCACTCTGTCGATACC |
| | CCACCGAGACCCCATTGGGGCCAAT |
| | ACGCCCGCGTTTCTTCCTTTTCCCC |
| | ACCCCACCCCCCAAGTTCGGGTGAA |
| | GGCCCAGGGCTCGCAGCCAACGTCG |
| | GGGCGGCAGGCCCTGCCATAG |
| | (SEQ ID NO: 263) |
| SNRP1 | GGTATCAAATAAAATACGAAATGTG |
| | ACAGATT (SEQ ID NO: 264) |
| SNRP1a | AAATAAAATACGAAATGTGACAGAT |
| | T (SEQ ID NO: 265) |
| Histone H4B | GGTTGCTGATTTCTCCACAGCTTGC |
| | ATTTCTGAACCAAAGGCCCTTTTCA |
| | GGGCCGCCCAACTAAACAAAAGAAG |
| | AGCTGTATCCATTAAGTCAAGAAGC |
| | (SEQ ID NO: 266) |
| MALAT-1 | GATTCGTCAGTAGGGTTGTAAAGGT |
| | TTTTCTTTTCCTGAGAAAACAACCT |
| | TTTGTTTTCTCAGGTTTTGCTTTTT |
| | GGCCTTTCCCTAGCTTTAAAAAAAA |
| | AAAAGCAAAAGACGCTGGTGGCTGG |
| | CACTCCTGGTTTCCAGGACGGGGTT |
| | CAAGTCCCTGCGGTGTCTTTGCTT |
| | (SEQ ID NO: 267) |
| MALAT-comp14 | AAAGGTTTTTCTTTTCCTGAGAAAT |
| | TTCTCAGGTTTTGCTTTTTAAAAAA |
| | AAAGCAAAAGACGCTGGTGGCTGGC |
| | ACTCCTGGTTTCCAGGACGGGGTTC |
| | AAGTCCCTGCGGTGTCTTTGCTT |
| | (SEQ ID NO: 268) |

In certain embodiments, the compact promoter is coupled with a viral intron (e.g., an SV40i intron, a MVM intron, a Mv2 intron, an HNRNPH1 intron, chimeric introns or synthetic introns).

In certain embodiments, the compact promoter does not comprise a viral promoter and/or a synthetic promoter.

In certain embodiments, the compact promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a compact promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### H1 Promoters

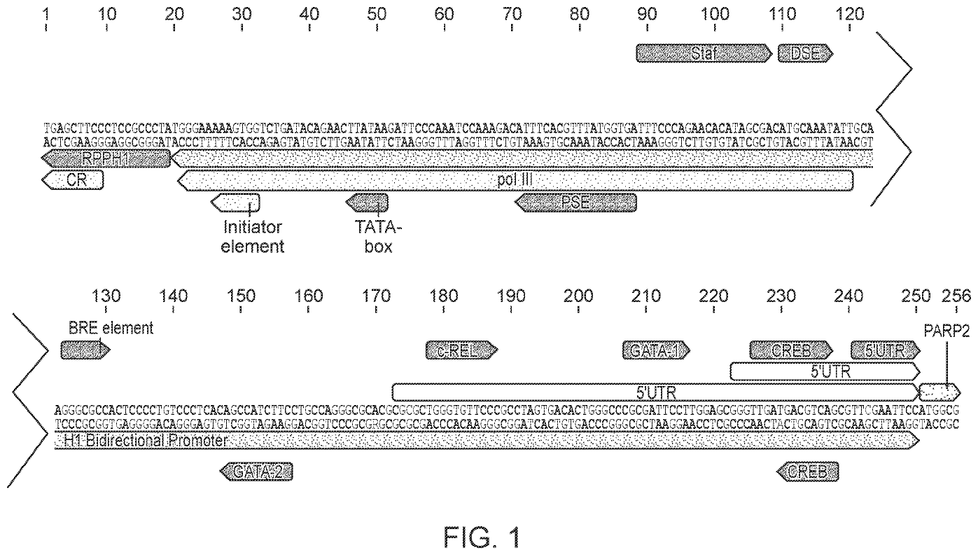

In certain embodiments, the promoter is comprises an H1 promoter. The H1 promoter is a bidirectional promoter having both pol II and pol III activity. The disclosure provides previously unidentified H1 promoters that Applicant identified by generating a Hidden Markov model (HMM) profile from a multispecies alignment of known H1 promoters (see, e.g., International Patent Publication No. WO2015/195621 and WO2018/009534). Regions flanking the H1 promoter region that were conserved throughout mammals were identified. As shown in FIG. 1., the region comprising the H1 promoter is located between the RPPH1 (H1 RNA) gene located on the minus strand to the left, and the beginning (i.e., the ATG(GCG)) of the protein coding gene, PARP2, located to the right. The RPPH1 gene comprises a highly conserved region in the H1 RNA gene (5′-GGAAGCTCA-3′) that is conserved throughout all mammals. Accordingly, in certain embodiments, the H1 promoter comprises or consists of a region between the ATG(GCG) of PARP2, and the highly conserved region in the H1 RNA gene (5′-GGAAGCTCA-3′). Also shown in FIG. 1 is the position of the pol III portion of the H1 promoter. Additional conserved regions present in the H1 promoter are shown, including, for example, conserved transcription factor binding sites, like a TATA box.

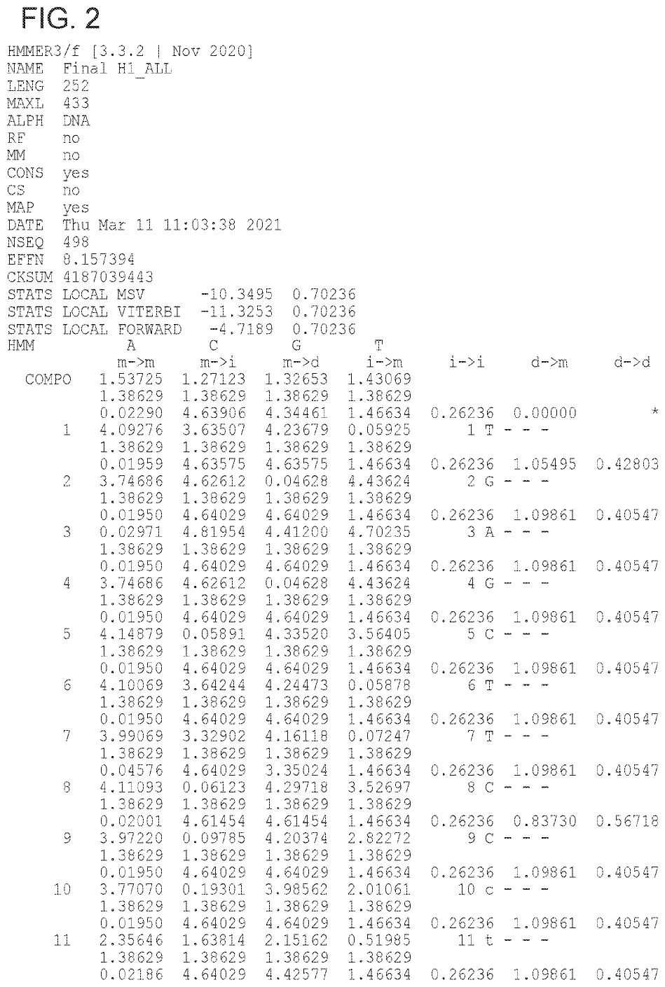

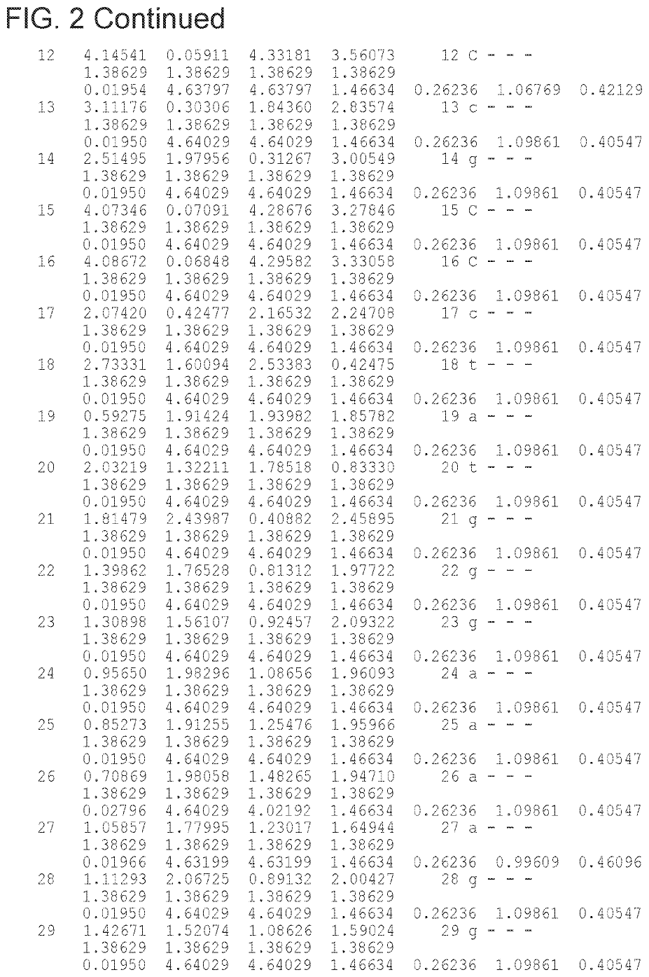

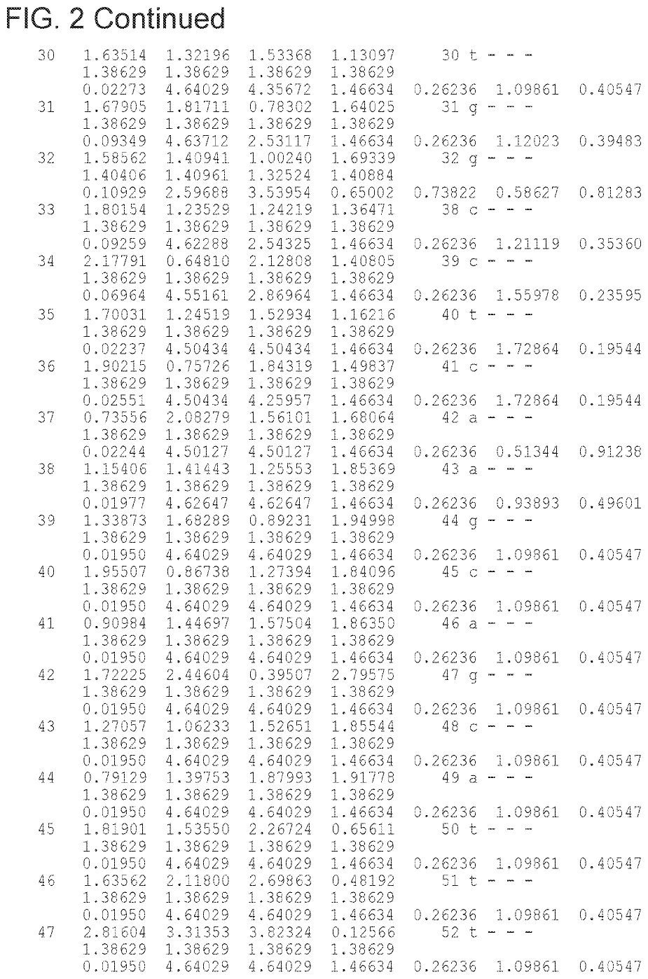

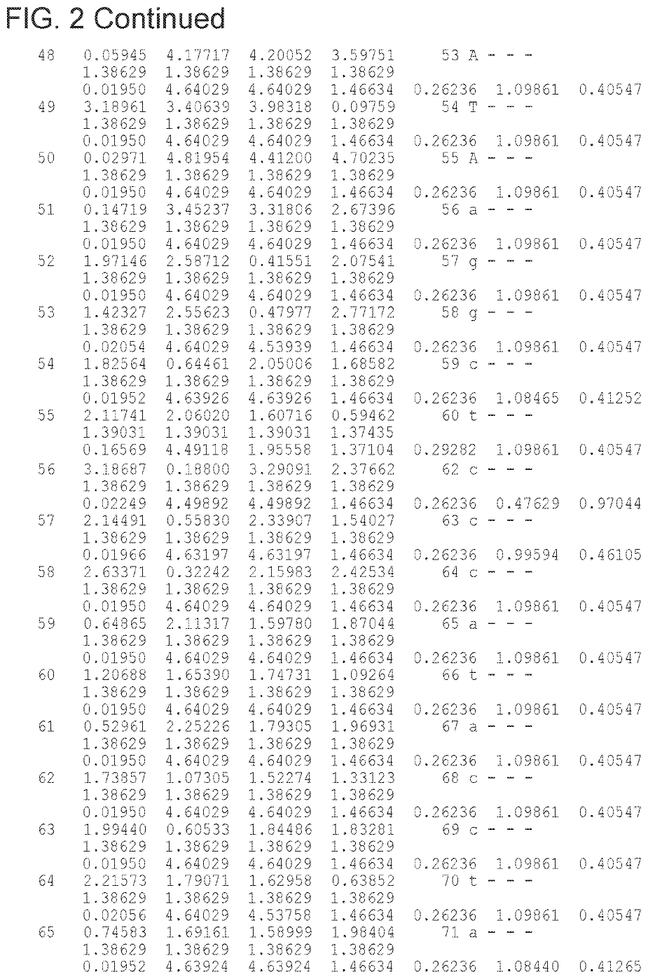

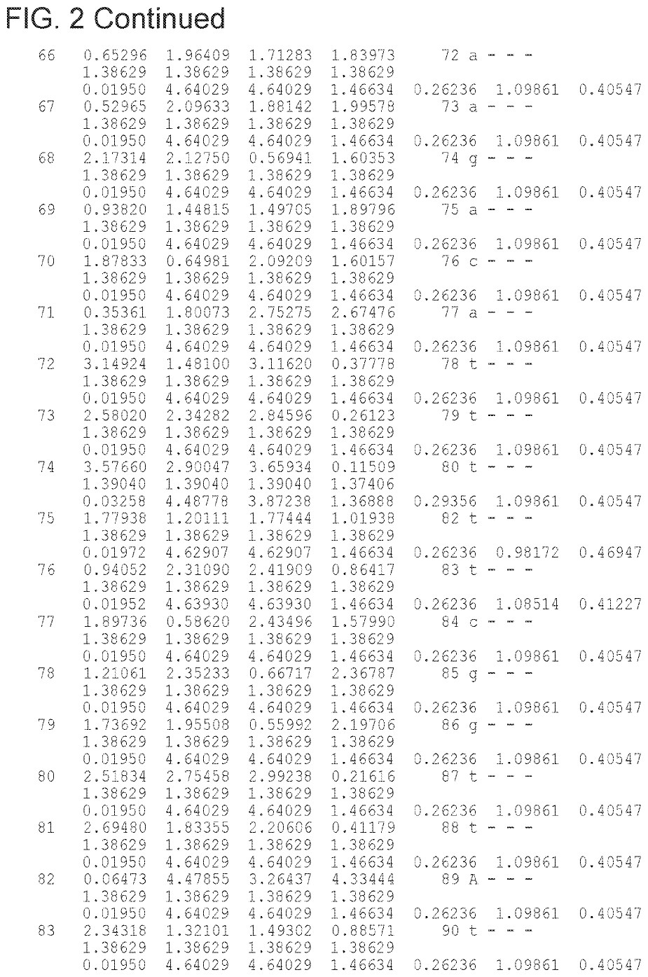

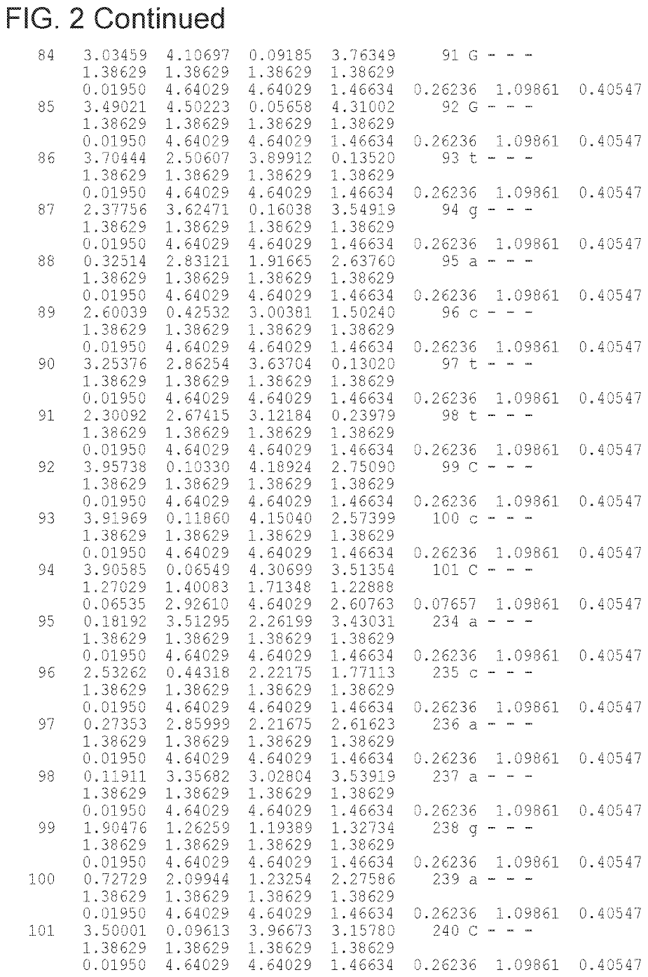

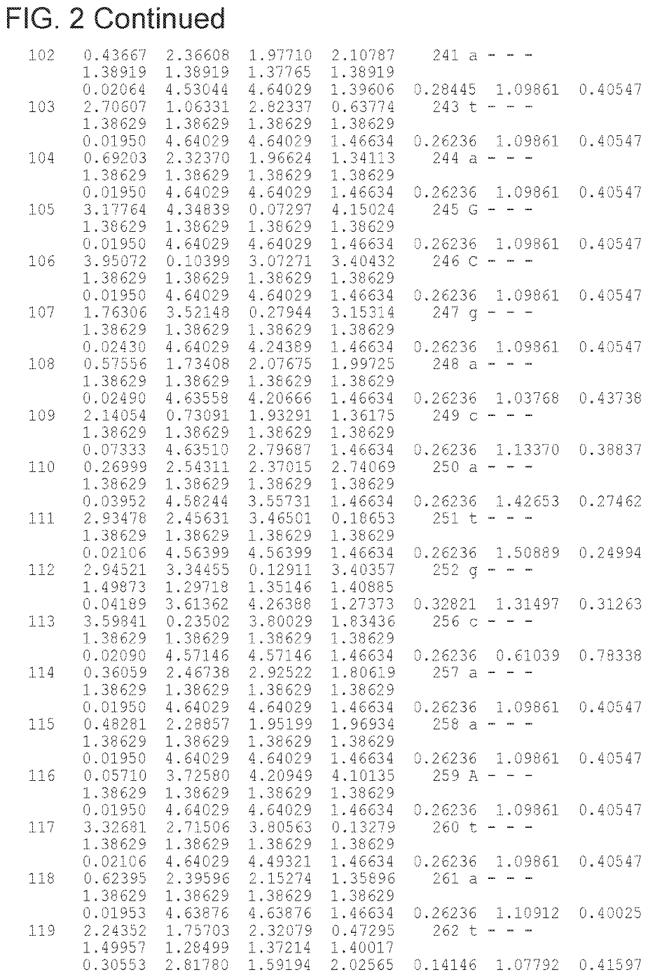

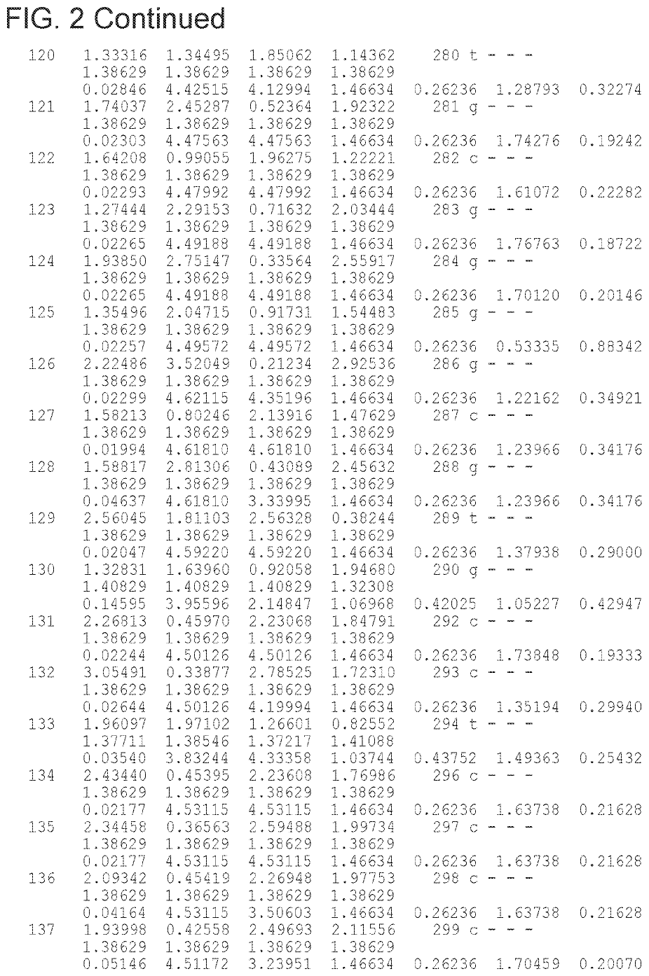

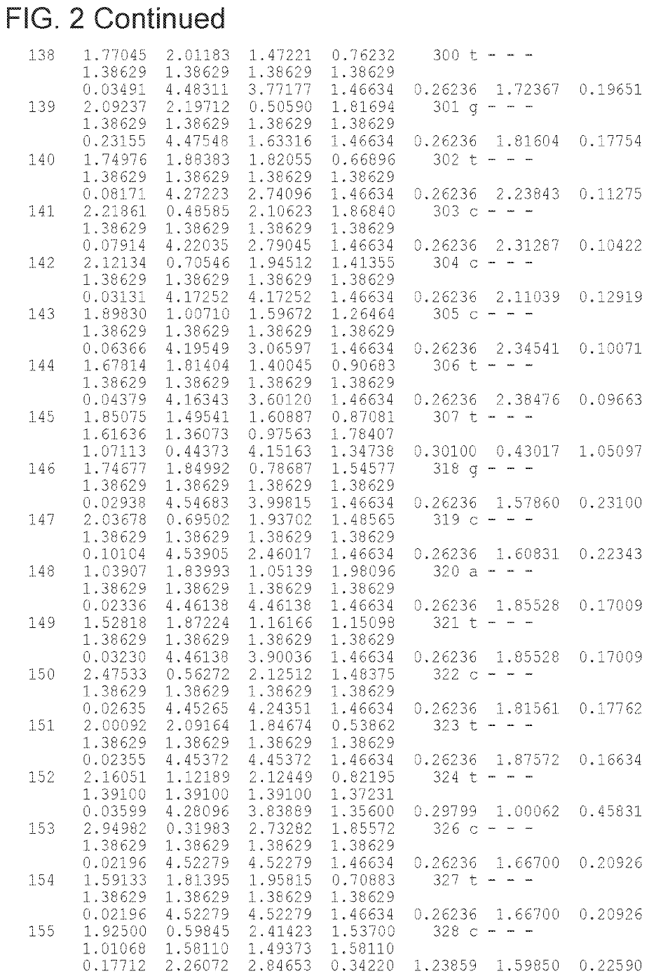

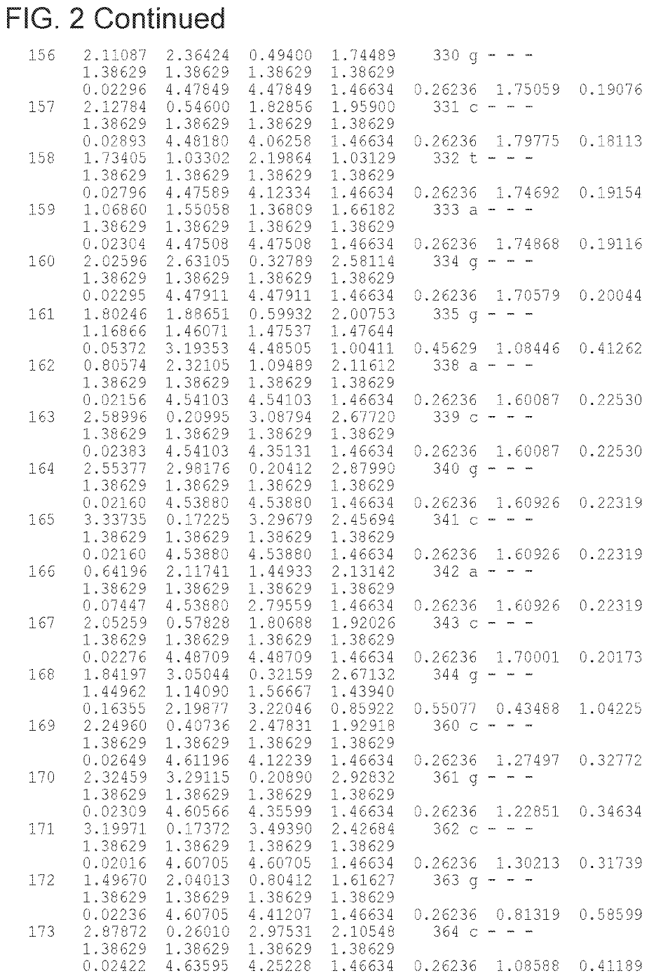

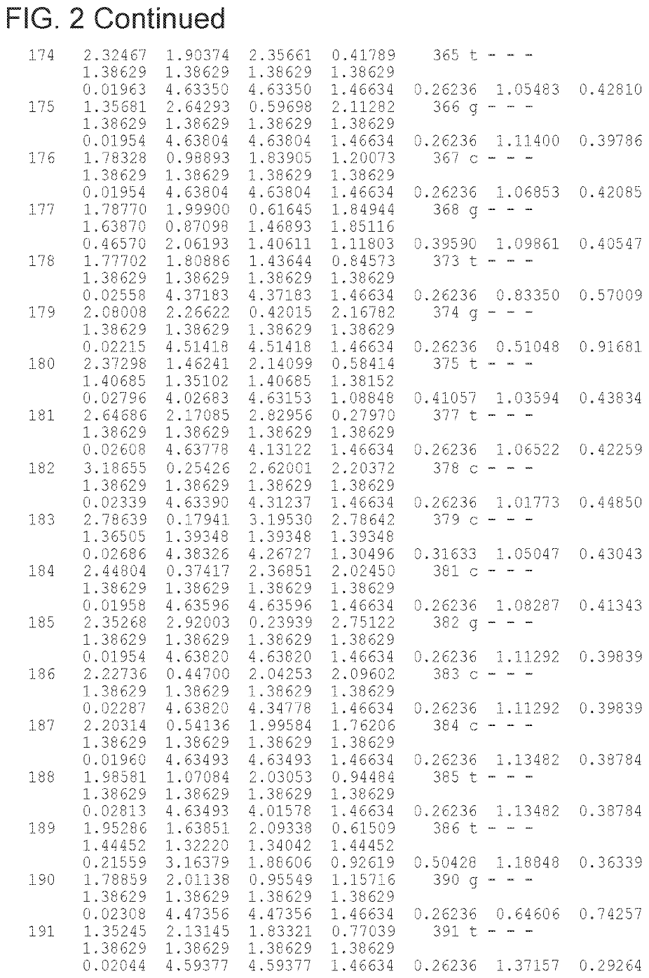

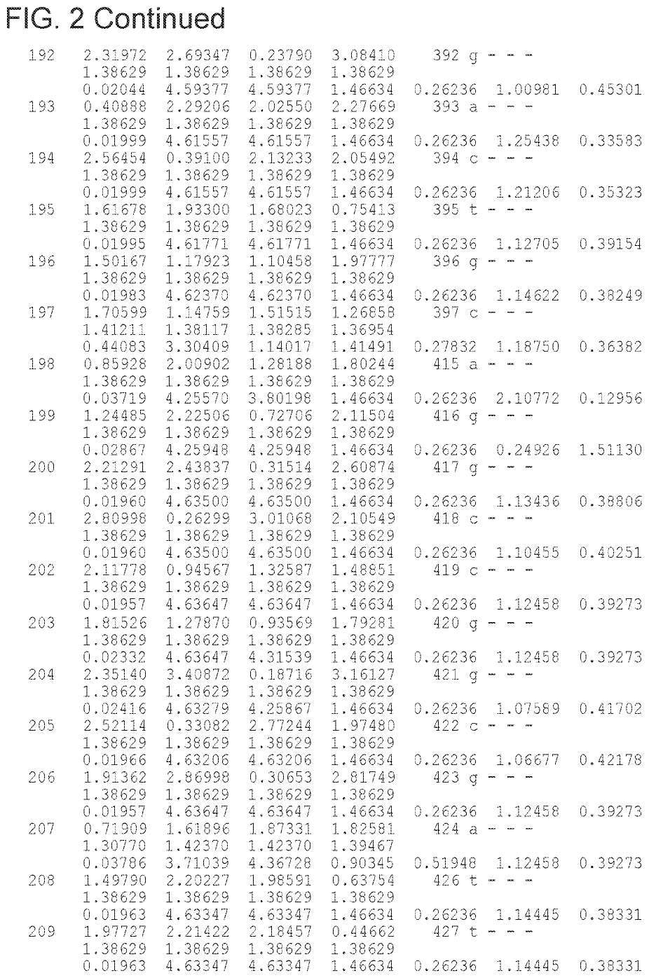

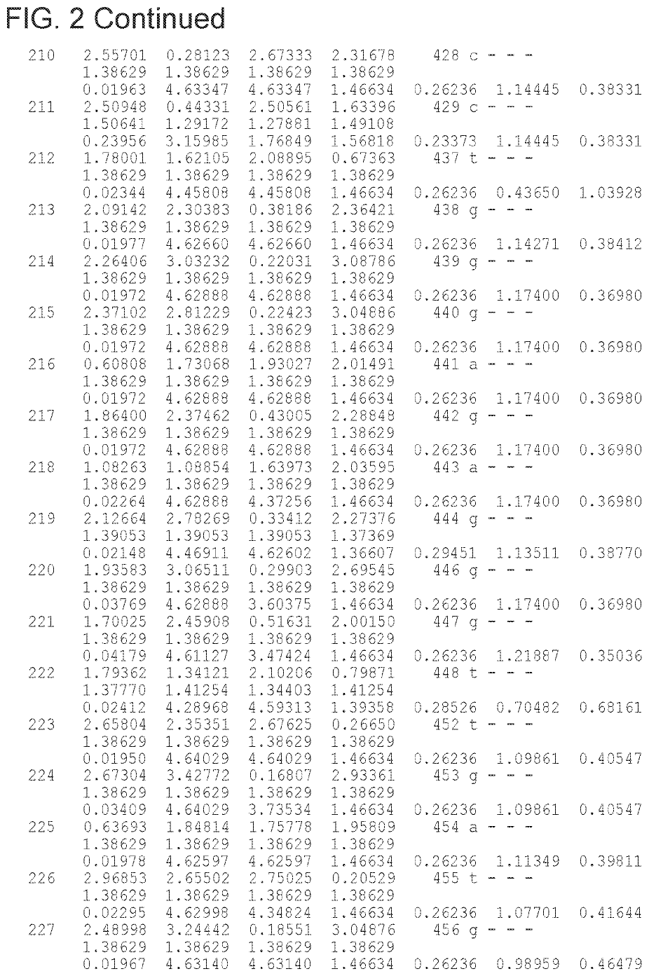

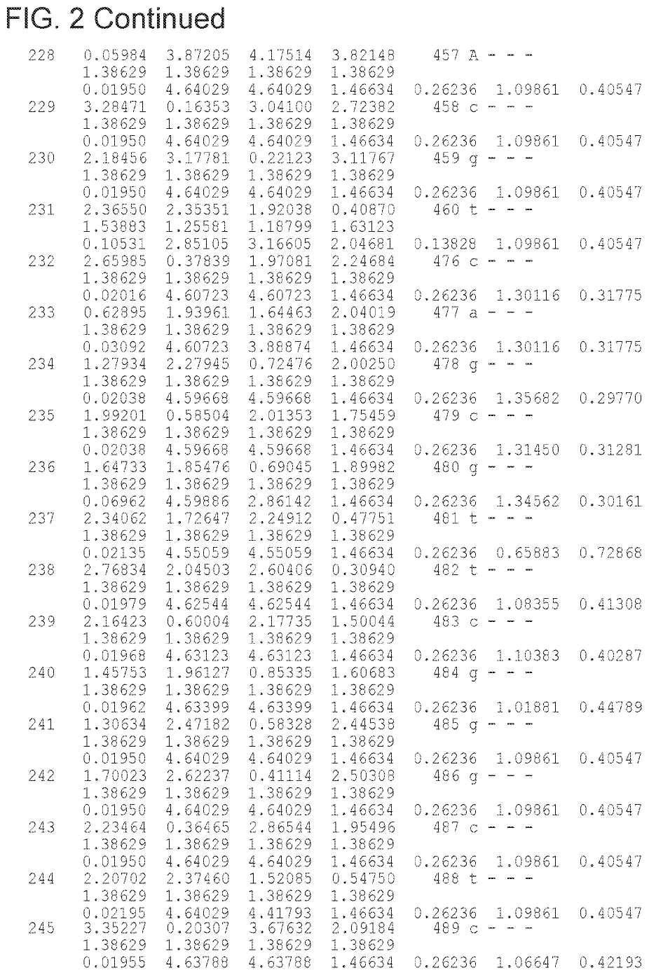

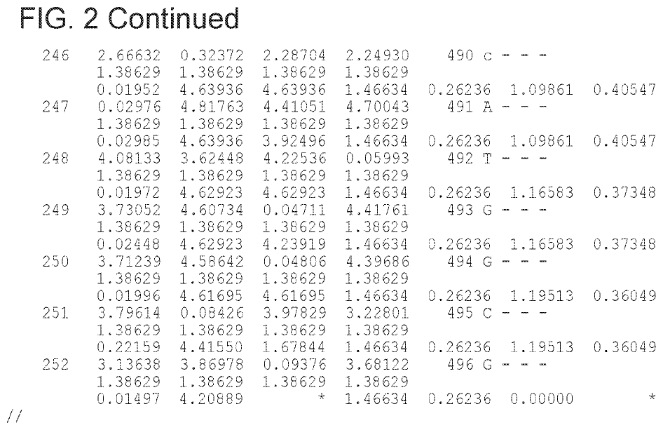

A Hidden Markov model (HMM) profile for identifying H1 promoters is provided in FIG. 2.

An alignment of naturally-occurring H1 promoters and consensus sequences is provided in FIG. 3 (wherein sequences numbered 1-498 in FIG. 3 correspond to SEQ ID NOs: 1304-1803 and consensus/100%, consensus/90%, consensus/80% and consensus/70% correspond to SEQ ID NOs: 1804-1807, respectively). Nucleotides 1-19 (as numbered in the alignment) form part of the H1 RNA gene and nucleotides 491 and above (as numbered in the alignment) form part of the PARP2 gene. Accordingly, nucleotides 20-490 correspond to the H1 promoter as used herein. Thus, in certain embodiments, the H1 promoter comprises nucleotides 20-490, as numbered in the alignment (or corresponding to the numbering in the alignment of FIG. 3 for a given H1 promoter sequence not present in the alignment of FIG. 3) of any one of SEQ ID NOs: 25-106 or a sequence provided in FIGS. 3-19. In addition, nucleotides 19-280, as numbered in the alignment (or corresponding to the numbering in the alignment of FIG. 3 for a given H1 promoter sequence not present in the alignment of FIG. 3)) of any one of SEQ ID NOs: 25-106 or a sequence provided in FIGS. 3-19 correspond with the pol III portion of the H1 promoter.

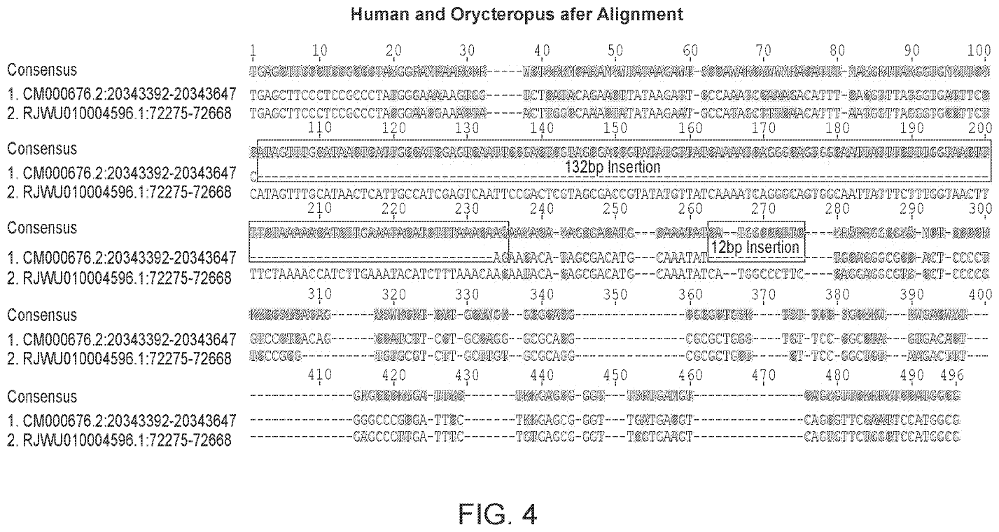

An alignment of human and Orycteropus afer (Aardvark) H1 promoter sequences provided in FIG. 4 shows a 132 bp and a 12 bp insertion found in the Orycteropus afer H1 promoter sequence. Without wishing to be bound by theory, it is noted that the 144 bp insertion corresponds closely to the length of DNA required to wrap around a nucleosome (147 bp). Therefore, given the context of DNA found in eukaryotic cells, binding site distances are maintained and conserved.

In certain embodiments, the promoter is selected from a promoter in TABLE 3.

TABLE 3

| Promoter | | SEQ |
|---|---|---|
| Designation | Promoter Name | ID NO: |
| p095 | Marmoset H1 Bidirectional Promoter | 91 |
| p127 | Big brown bat H1 Bidirectional Promoter | 27 |
| p094 | Microbat H1 Bidirectional Promoter | 49 |
| p071 | Synthetic-2 H1 Bidirectional Promoter | 63 |
| p110 | Elephant H1 Bidirectional Promoter | 80 |
| p101 | Opossum H1 Bidirectional Promoter | 50 |
| p109 | David's myotis H1 Bidirectional Promoter | 38 |
| p116 | Bushbaby H1 Bidirectional Promoter | 74 |
| p066 | Star-nosed mole H1 Bidirectional Promoter | 61 |
| p060 | Tree Shrew H1 Bidirectional Promoter | 66 |
| p099 | Guinea pig H1 Bidirectional Promoter | 85 |
| p131 | Aardvark H1 Bidirectional Promoter | 25 |
| p100 | Goat H1 Bidirectional Promoter | 41 |
| p098 | Ferret H1 Bidirectional Promoter | 82 |
| p097 | Horse H1 Bidirectional Promoter | 86 |
| p092 | Killer whale H1 Bidirectional Promoter | 45 |
| p073 | Shrew H1 Bidirectional Promoter | 56 |
| p112 | Chinese tree shrew H1 Bidirectional Promoter | 36 |
| p081 | Sooty mangabey H1 Bidirectional Promoter | 59 |
| p078 | Shrew mouse H1 Bidirectional Promoter | 57 |
| p079 | Sheep H1 Bidirectional Promoter | 102 |
| p077 | Sifaka H1 Bidirectional Promoter | 58 |
| p065 | White-faced sapajou H1 Bidirectional Promoter | 69 |
| p130 | Angolan colobus H1 Bidirectional Promoter | 26 |
| p084 | Rat H1 Bidirectional Promoter | 100 |
| p106 | Cape golden mole H1 Bidirectional Promoter | 33 |
| p088 | Orangutan H1 Bidirectional Promoter | 95 |
| p091 | Mas night monkey H1 Bidirectional Promoter | 48 |
| p103 | Manatee H1 Bidirectional Promoter | 47 |
| p102 | Large flying fox H1 Bidirectional Promoter | 89 |
| p087 | Golden hamster H1 Bidirectional Promoter | 42 |
| p083 | Squirrel monkey H1 Bidirectional Promoter | 60 |
| p063 | Weddell seal H1 Bidirectional Promoter | 67 |
| p064 | Tenrec H1 Bidirectional Promoter | 64 |
| p072 | Pig H1 Bidirectional Promoter | 97 |
| p070 | Ryukyu mouse H1 Bidirectional Promoter | 55 |
| p119 | Cat H1 Bidirectional Promoter | 75 |
| p082 | Tarsier H1 Bidirectional Promoter | 104 |
| p059 | Mouse H1 Bidirectional Promoter | 92 |
| p058 | Panda H1 Bidirectional Promoter | 96 |
| p085 | Rhesus H1 Bidirectional Promoter | 54 |
| p062 | White rhinoceros H1 Bidirectional Promoter | 68 |
| p067 | Pig-tailed macaque H1 Bidirectional Promoter | 52 |
| p107 | Black flying-fox H1 Bidirectional Promoter | 28 |
| p061 | Tibetan antelope H1 Bidirectional Promoter | 65 |
| p086 | Gorilla H1 Bidirectional Promoter | 83 |
| p105 | Hedgehog H1 Bidirectional Promoter | 44 |
| p089 | Golden snub-nosed monkey H1 Bidirectional | 43 |
| | Promoter | |
| p096 | Human H1 Bidirectional Promoter | 87 |
| p090 | Gibbon H1 Bidirectional Promoter | 40 |
| p076 | Pacific walrus H1 Bidirectional Promoter | 51 |
| p113 | Crab-eating macaque H1 Bidirectional Promoter | 78 |
| p069 | Synthetic-1 H1 Bidirectional Promoter | 62 |
| p068 | Squirrel H1 Bidirectional Promoter | 103 |
| p093 | Lesser Egyptian jerboa H1 Bidirectional Promoter | 46 |
| p074 | Rabbit H1 Bidirectional Promoter | 99 |
| p125 | Chimp H1 Bidirectional Promoter | 76 |
| p124 | Brush-tailed rat H1 Bidirectional Promoter | 31 |
| p117 | Chinese hamster H1 Bidirectional Promoter | 35 |
| p114 | Drill H1 Bidirectional Promoter | 39 |
| p108 | Camel H1 Bidirectional Promoter | 32 |
| p118 | Consensus-1 H1 Bidirectional Promoter | 37 |
| p126 | Baboon H1 Bidirectional Promoter | 72 |
| p129 | Armadillo H1 Bidirectional Promoter | 71 |
| p111 | Black snub-nosed monkey H1 Bidirectional | 29 |
| | Promoter | |
| p122 | Bonobo H1 Bidirectional Promoter | 30 |
| p120 | Bottlenose dolphin H1 Bidirectional Promoter | 73 |
| p128 | Alpaca H1 Bidirectional Promoter | 70 |
| p104 | Green monkey H1 Bidirectional Promoter | 84 |
| p123 | Chinchilla H1 Bidirectional Promoter | 34 |
| p115 | Cow H1 Bidirectional Promoter | 77 |

In certain embodiments, the H1 promoter is a mammalian promoter, e.g., an artiodactyla H1 promoter, a carnivora H1 promoter, a cetacea H1 promoter, a chiroptera H1 promoter, an insectivora H1 promoter, a lagomorpha H1 promoter, a marsupial H1 promoter, a pangolin H1 promoter, a perissodactyla H1 promoter, a primate H1 promoter, a rodent H1 promoter, or a xenartha promoter. In certain embodiments, the H1 promoter is an ancestral promoter (e.g., selected from SEQ ID NOs: 936-1303). In some embodiments, the promoter comprises a nucleotide sequence having at least 85%, 90%, 95%, 96%, 97%, 98%, 99%, or 100% identity to the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490 as numbered in FIG. 3), or a functional fragment or variant (e.g., codon optimized) thereof. In some embodiments, the promoter comprises the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or the portion of any sequence in FIGS. 3-19 that corresponds to the H1 promoter (e.g., from about nucleotide 20 to about nucleotide 490) as numbered in FIG. 3), or a functional fragment or variant (e.g., codon optimized) thereof.

In certain embodiments, the promoter is not one or more of an alpaca H1 promoter (SEQ ID NO: 70), an armadillo H1 promoter (SEQ ID NO: 71), a baboon H1 promoter (SEQ ID NO: 72), a bottlenose dolphin H1 promoter (SEQ ID NO: 73), a bushbaby H1 promoter (SEQ ID NO: 74), a cat H1 promoter (SEQ ID NO: 75), a chimp H1 promoter (SEQ ID NO: 76), a cow H1 promoter (SEQ ID NO: 77), a crab-eating macaque H1 promoter (SEQ ID NO: 78), a dog H1 promoter (SEQ ID NO: 79), an elephant H1 promoter (SEQ ID NO: 80), a European hedgehog H1 promoter (SEQ ID NO: 81), a ferret H1 promoter (SEQ ID NO: 82), a gorilla H1 promoter (SEQ ID NO: 83), a green monkey H1 promoter (SEQ ID NO: 84), a guinea pig H1 promoter (SEQ ID NO: 85), a horse H1 promoter (SEQ ID NO: 86), a human H1 promoter (SEQ ID NO: 87), a kangaroo rat H1 promoter (SEQ ID NO: 88), a large flying fox H1 promoter (SEQ ID NO: 89), a little brown bat H1 promoter (SEQ ID NO: 90), a marmoset H1 promoter (SEQ ID NO: 91), a mouse H1 promoter (SEQ ID NO: 92 or SEQ ID NO: 93), a northern treeshrew H1 promoter (SEQ ID NO: 94), an orangutan H1 promoter (SEQ ID NO: 95), a panda H1 promoter (SEQ ID NO: 96), a pig H1 promoter (SEQ ID NO: 97), a pika H1 promoter (SEQ ID NO: 98), a rabbit H1 promoter (SEQ ID NO: 99), a rat H1 promoter (SEQ ID NO: 100), a rock hyax H1 promoter (SEQ ID NO: 101), a sheep H1 promoter (SEQ ID NO: 102), a squirrel H1 promoter (SEQ ID NO: 103), a tarsier H1 promoter (SEQ ID NO: 104), a two-toed sloth H1 promoter (SEQ ID NO: 105), or a white cheeked gibbon H1 promoter (SEQ ID NO: 106).

In certain embodiments, a functional fragment comprises a truncation of from about 10 bases to about 40 bases at the 5′ end, the 3′ end, or both the 5′ and 3′ ends of any one of SEQ ID NOs: 25-106 or a sequence provided in FIGS. 3-19, or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 15 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 20 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 0) 96%, 97%, 98%, 99%, or 100% identity to any one of SEQ ID NOs: 25-106 or a sequence provided in FIGS. 3-19). In certain embodiments, a functional fragment comprises a truncation of about 25 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 30 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19). In certain embodiments, a functional fragment comprises a truncation of about 35 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of any one of SEQ ID NOs: 25-106 or a sequence provided in FIGS. 3-19). In certain embodiments, a functional fragment comprises a truncation of about 40 bases at the 5′ end, the 3′ end, or both the 5′ and 3′ ends of any one of SEQ ID NOs: 25-106 or a sequence provided in FIGS. 3-19 or a variant thereof (e.g., a variant having at least 85%, 90%, 95%, 96%, 97%, 98%, 99%, or 100% identity to any one of SEQ ID NOs: 25-106 or a sequence provided in FIGS. 3-19).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83.

In certain embodiments, the promoter comprises a TATA mutation. In certain embodiments, the TATA mutation is a TATAA→TCGAA mutation.

In certain embodiments, a nucleic acid comprising a promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a promoter described herein further comprises a terminator sequence. In certain embodiments, the terminator sequence comprises one of the terminator sequences in TABLE 4.

TABLE 4

| | |
|---|---|
| a synthetic | AATAAAATATCTTTATTTTCATTAC |
| poly(A) | ATCTGTGTGTTGGTTTTTTGTGTG |
| sequence (SPA) | (SEQ ID NO: 258) |
| SPA and Pause | AATAAAATATCTTTATTTTCATTAC |
| | ATCTGTGTGTTGGTTTTTTGTGTGA |
| | ATCGATAGTACTAACATACGCTCTC |
| | CATCAAAACAAAACGAAACAAAACA |
| | AACTAGCAAAATAGGCTGTCCCCAG |
| | TGCAAGTGCAGGTGCCAGAACATTT |
| | CTCT (SEQ ID NO: 259); |
| SV40 (240bp) | ATCTAGATAACTGATCATAATCAGC |
| | CATACCACATTTGTAGAGGTTTTAC |
| | TTGCTTTAAAAAACCTCCCACACCT |
| | CCCCCTGAACCTGAAACATAAAATG |
| | AATGCAATTGTTGTTGTTAACTTGT |
| | TTATTGCAGCTTATAATGGTTACAA |
| | ATAAAGCAATAGCATCACAAATTTC |
| | ACAAATAAAGCATTTTTTTCACTGC |
| | ATTCTAGTTGTGGTTTGTCCAAACT |
| | CATCAATGTATCTTA |
| | (SEQ ID NO: 260) |
| SV 40-mini | TTGTTTATTGCAGCTTATAATGGTT |
| (120bp) | ACAAATAAAGCAATAGCATCACAAA |
| | TTTCACAAATAAAGCATTTTTTTCA |
| | CTGCATTCTAGTTGTGGTTTGTCCA |
| | AACTCATCAATGTATCTTAT |
| | (SEQ ID NO: 261) |
| bGH poly A | CGACTGTGCCTTCTAGTTGCCAGCC |
| | ATCTGTTGTTTGCCCCTCCCCCGTG |
| | CCTTCCTTGACCCTGGAAGGTGCCA |
| | CTCCCACTGTCCTTTCCTAATAAAA |
| | TGAGGAAATTGCATCGCATTGTCTG |
| | AGTAGGTGTCATTCTATTCTGGGGG |
| | GTGGGGTGGGGCAGGACAGCAAGGG |
| | GGAGGATTGGGAAGACAATAGCAGG |
| | CATGCTGGGGATGCGGTGGGCTCTA |
| | TGG (SEQ ID NO: 262) |
| TKpoly A | GGGGGAGGCTAACTGAAACACGGAA |
| | GGAGACAATACCGGAAGGAACCCGC |
| | GCTATGACGGCAATAAAAAGACAGA |
| | ATAAAACGCACGGGTGTTGGGTCGT |
| | TTGTTCATAAACGCGGGGTTCGGTC |
| | CCAGGGCTGGCACTCTGTCGATACC |
| | CCACCGAGACCCCATTGGGGCCAAT |
| | ACGCCCGCGTTTCTTCCTTTTCCCC |
| | ACCCCACCCCCCAAGTTCGGGTGAA |
| | GGCCCAGGGCTCGCAGCCAACGTCG |
| | GGGCGGCAGGCCCTGCCATAG |
| | (SEQ ID NO: 263) |
| sNRP1 | GGTATCAAATAAAATACGAAATGTG |
| | ACAGATT (SEQ ID NO: 264) |
| sNRP1a | AAATAAAATACGAAATGTGACAGAT |
| | T (SEQ ID NO: 265) |
| Histone H4B | GGTTGCTGATTTCTCCACAGCTTGC |
| | ATTTCTGAACCAAAGGCCCTTTTCA |
| | GGGCCGCCCAACTAAACAAAAGAAG |
| | AGCTGTATCCATTAAGTCAAGAAGC |
| | (SEQ ID NO: 266) |
| MALAT-1 | GATTCGTCAGTAGGGTTGTAAAGGT |
| | TTTTCTTTTCCTGAGAAAACAACCT |
| | TTTGTTTTCTCAGGTTTTGCTTTTT |
| | GGCCTTTCCCTAGCTTTAAAAAAAA |
| | AAAAGCAAAAGACGCTGGTGGCTGG |
| | CACTCCTGGTTTCCAGGACGGGGTT |
| | CAAGTCCCTGCGGTGTCTTTGCTT |
| | (SEQ ID NO: 267) |
| MALAT-comp14 | AAAGGTTTTTCTTTTCCTGAGAAAT |
| | TTCTCAGGTTTTGCTTTTTAAAAAA |
| | AAAGCAAAAGACGCTGGTGGCTGGC |
| | ACTCCTGGTTTCCAGGACGGGGTTC |
| | AAGTCCCTGCGGTGTCTTTGCTT |
| | (SEQ ID NO: 268) |

In certain embodiments, the compact promoter is coupled with a viral intron (e.g., an SV40i intron, a MVM intron, a Mv2 intron, an HNRNPH1 intron, chimeric introns or synthetic introns.).

In certain embodiments, the compact promoter does not comprise a viral promoter and/or a synthetic promoter.

In certain embodiments, the compact promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a compact promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### Artiodactyla H1 Promoters

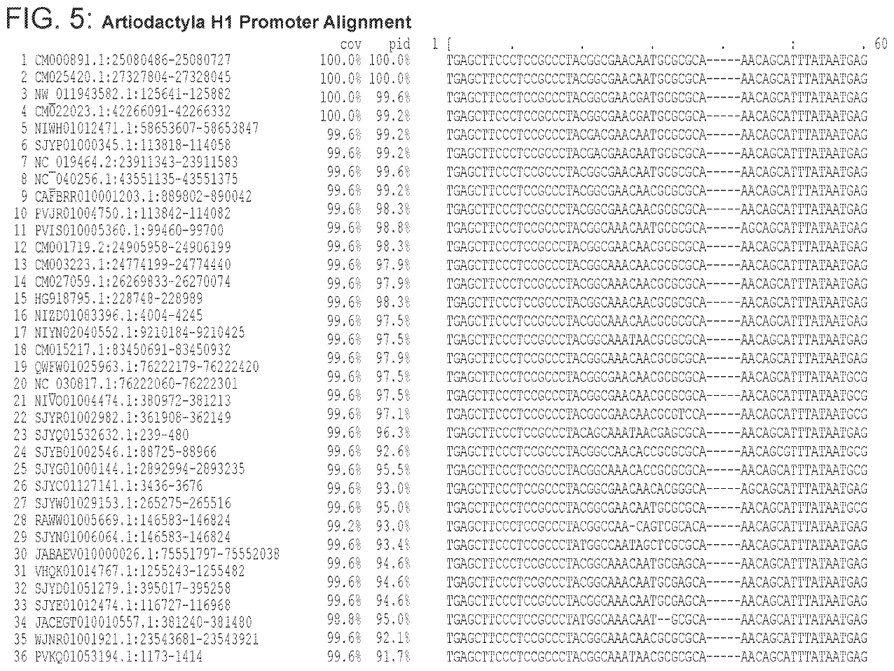

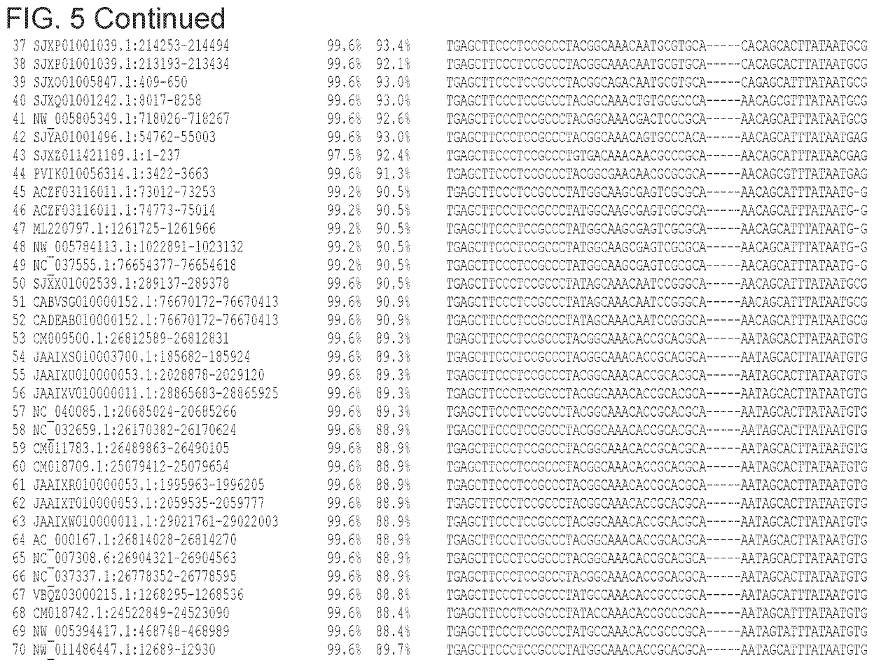

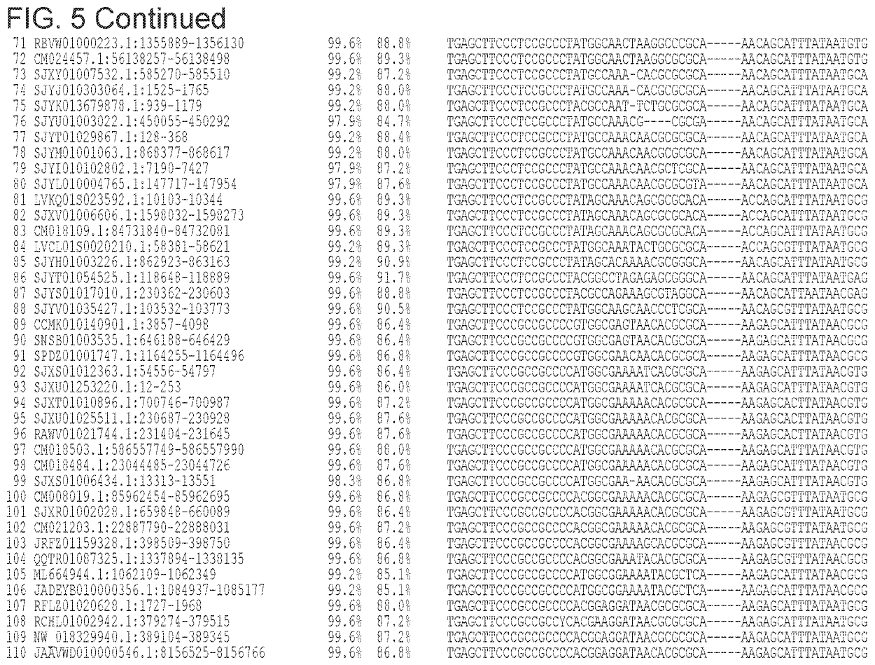

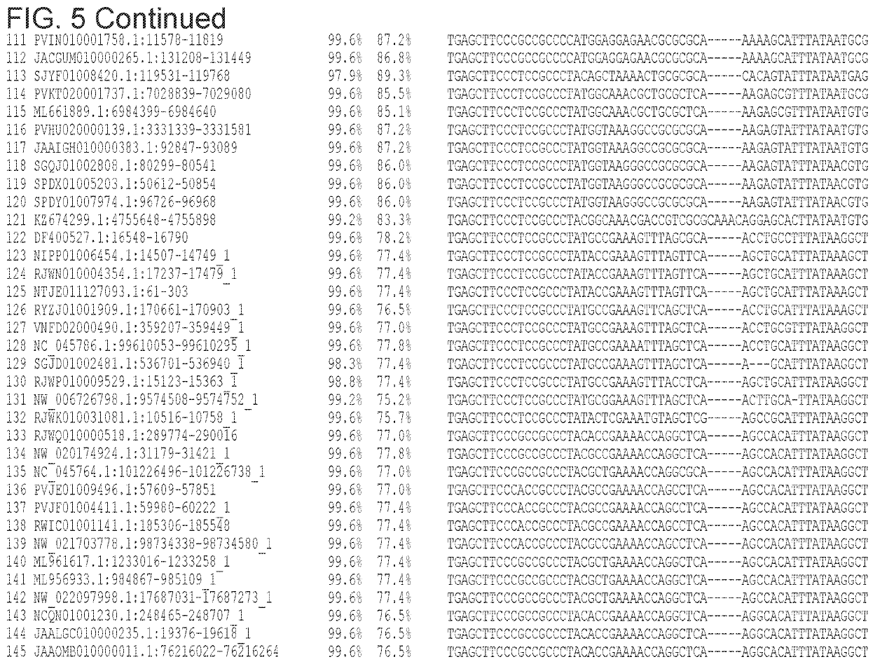

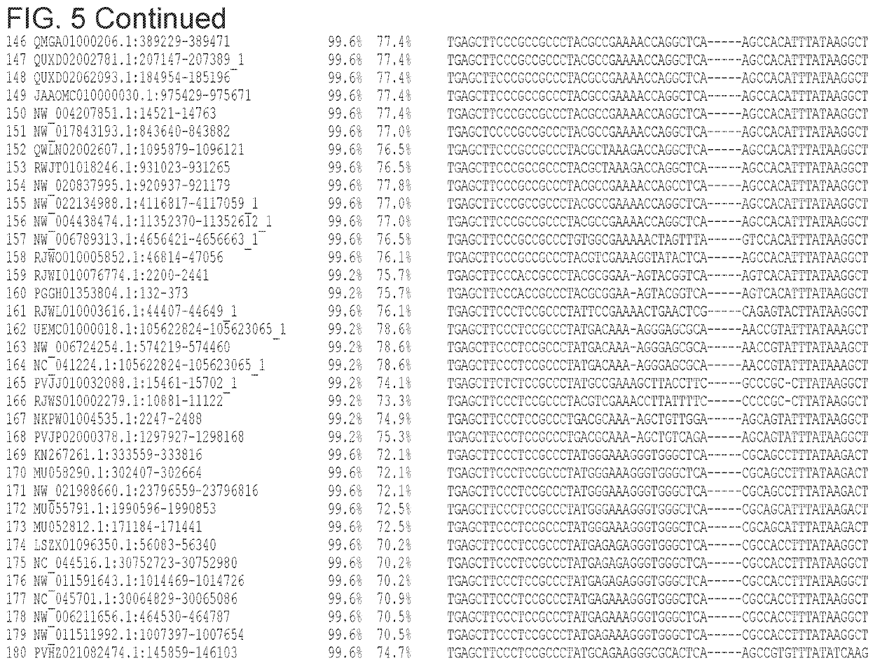

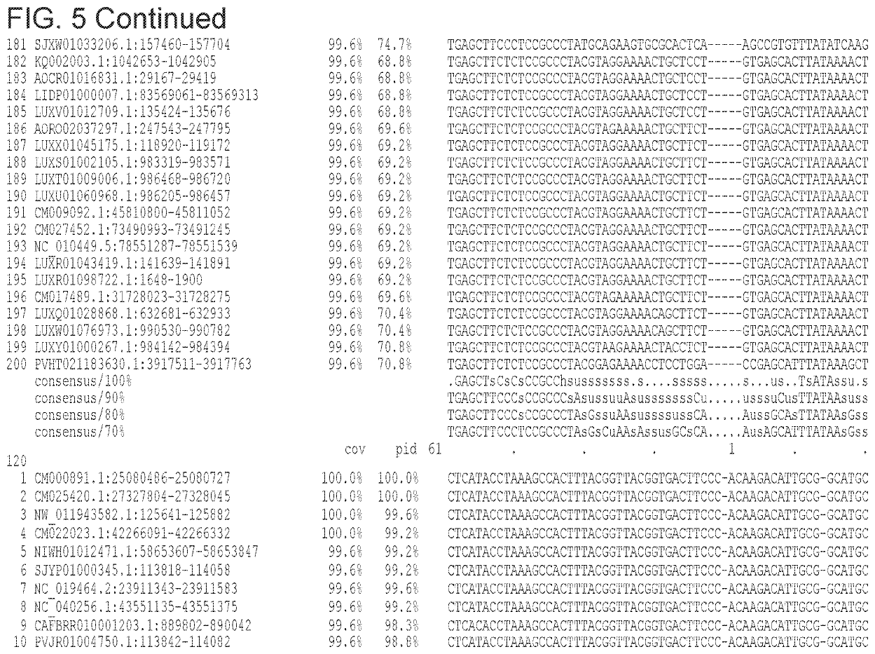

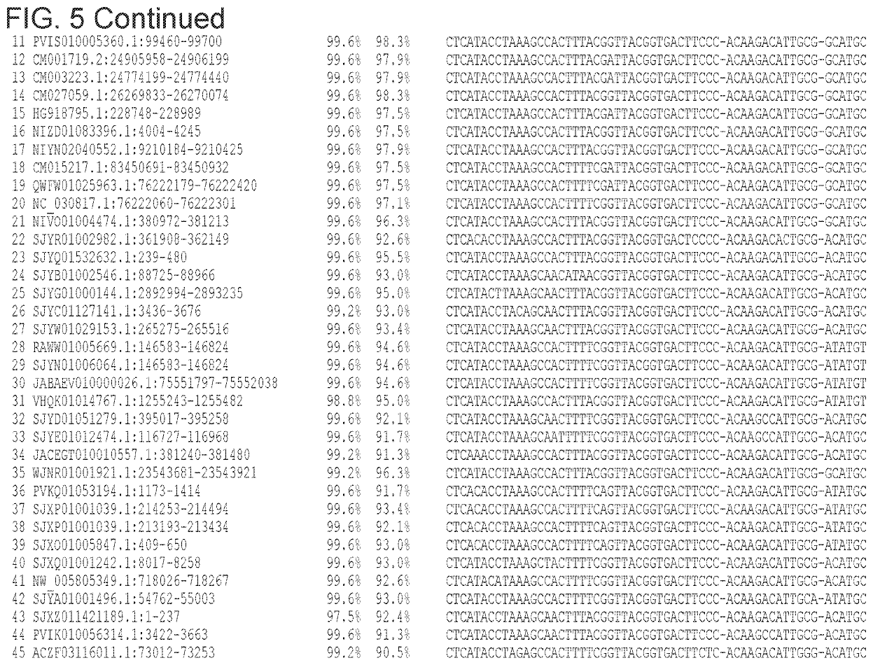

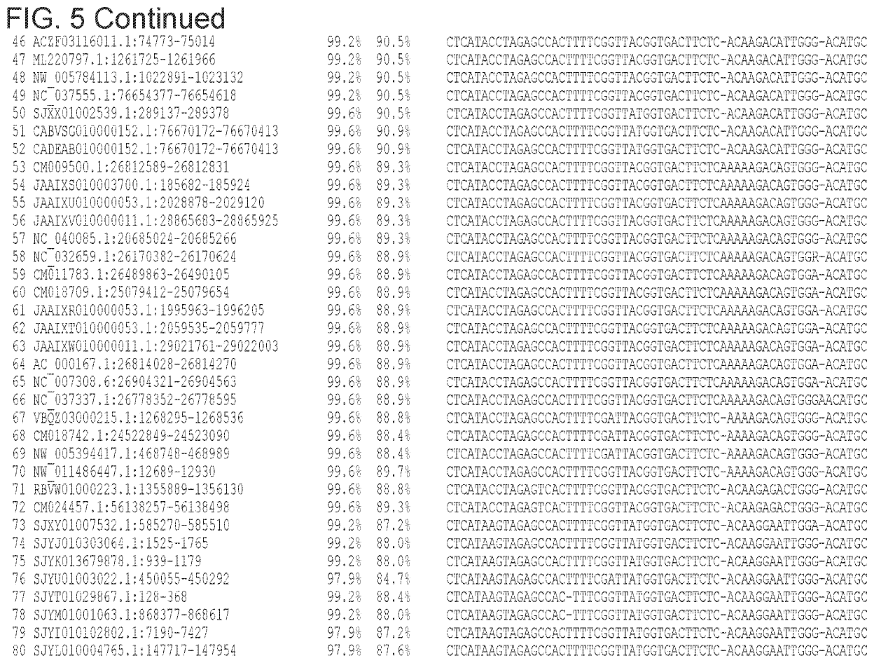

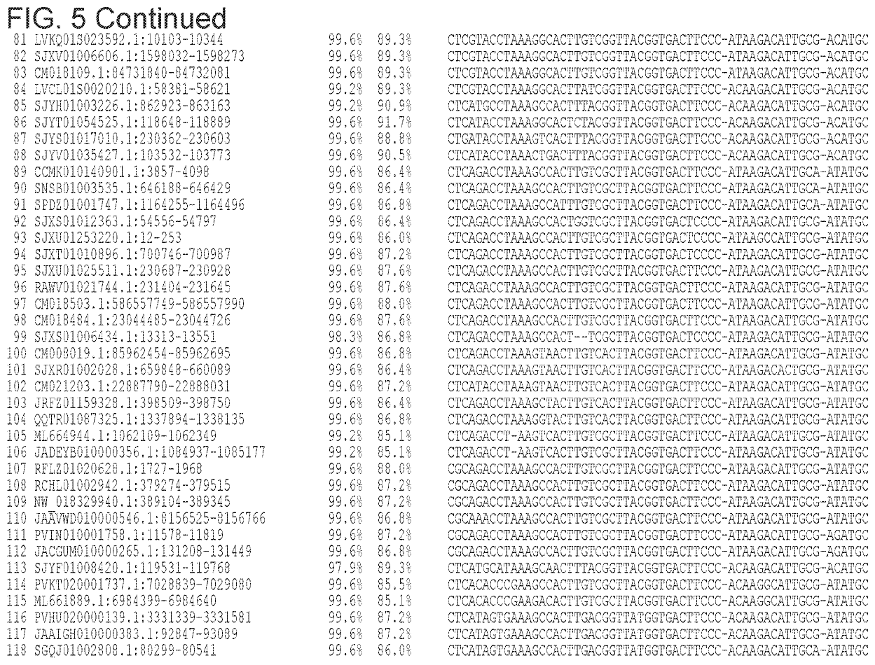

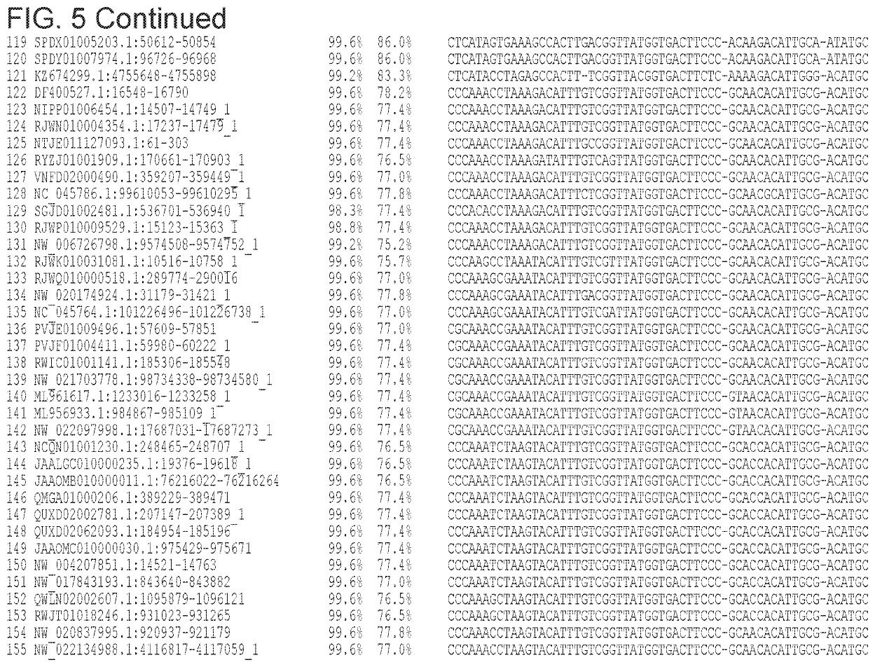

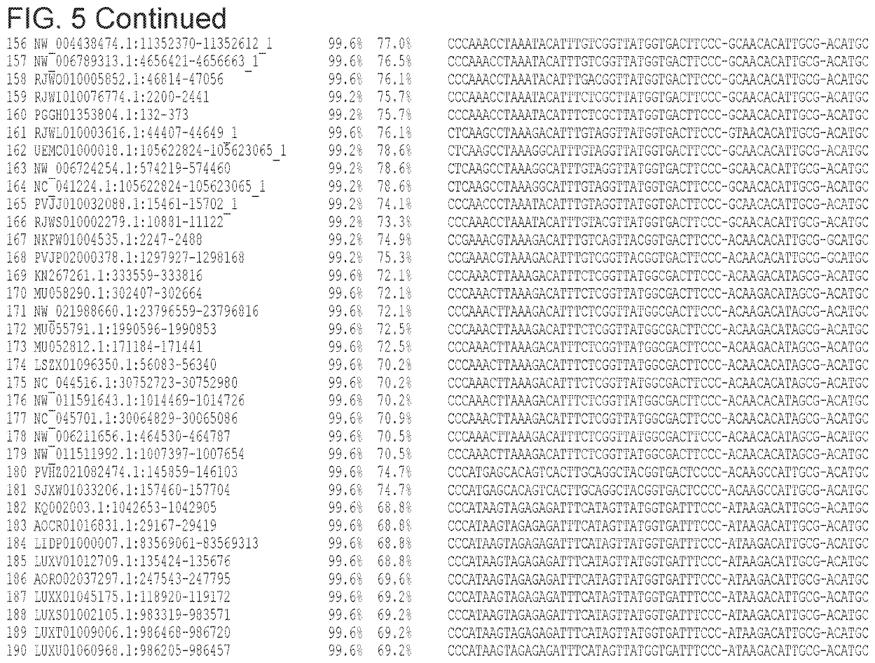

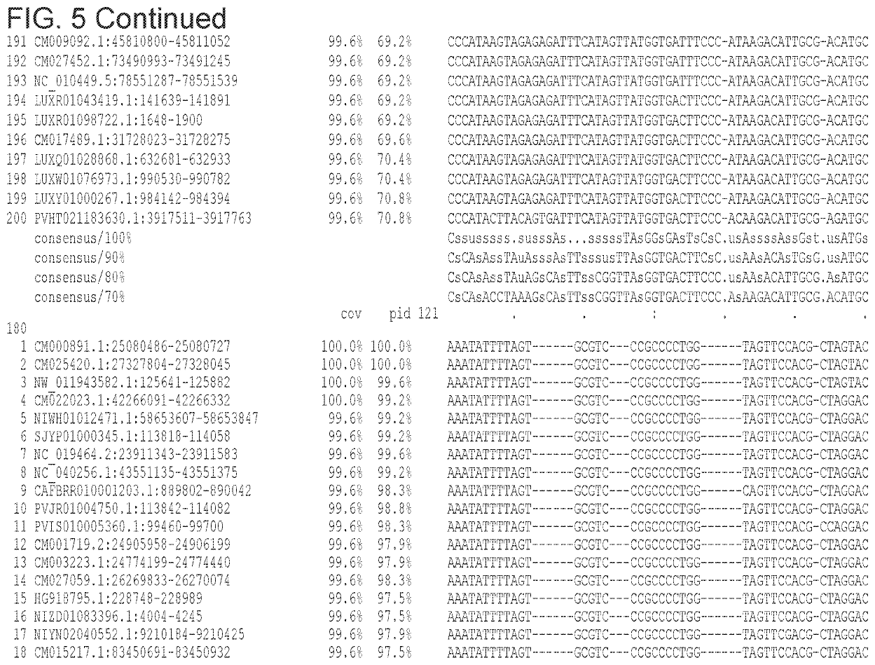

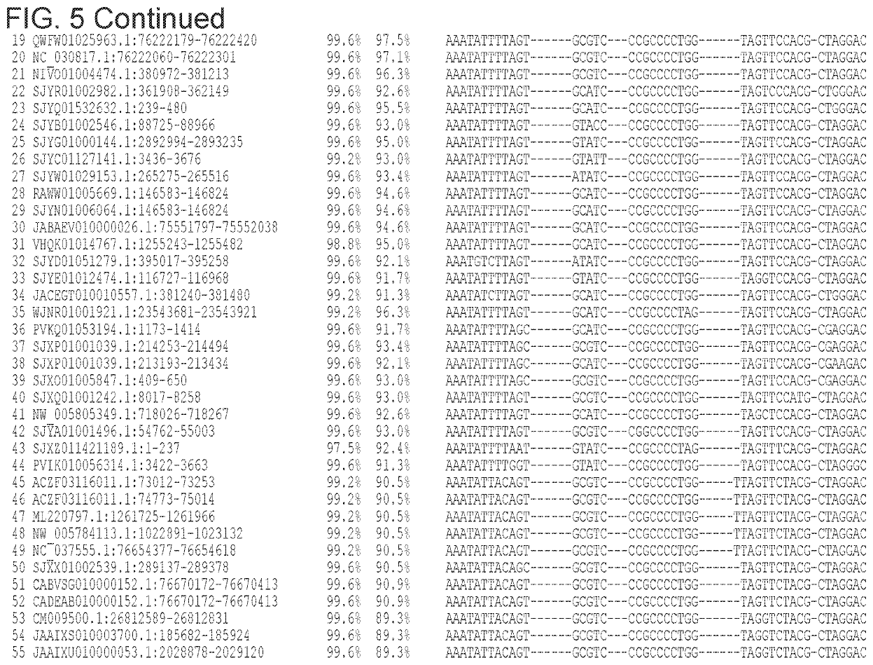

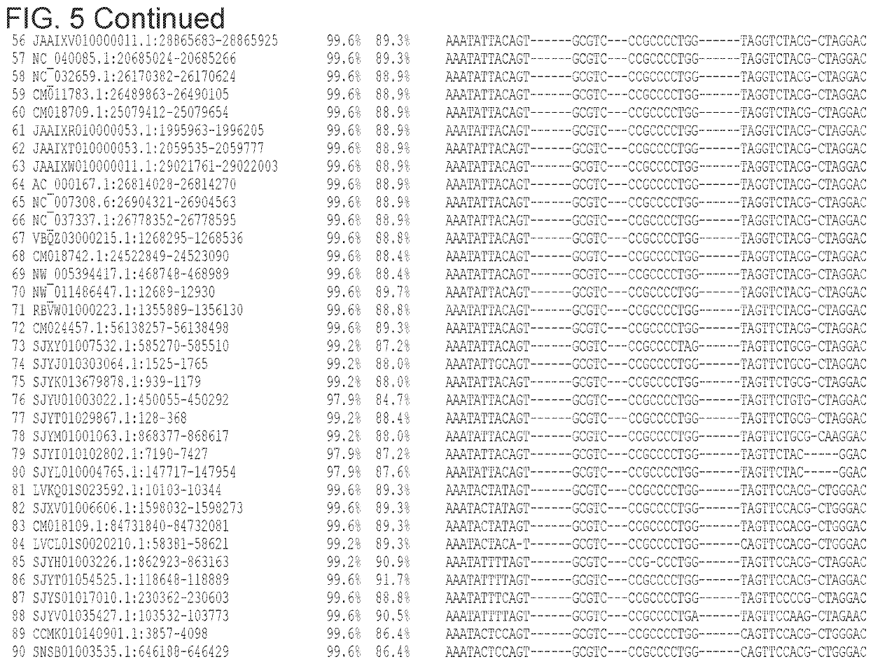

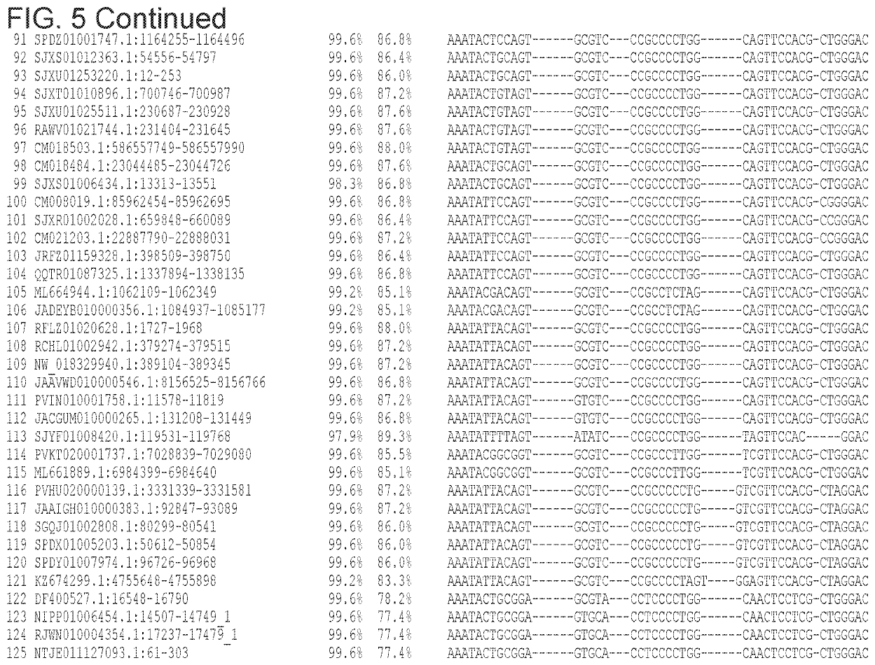

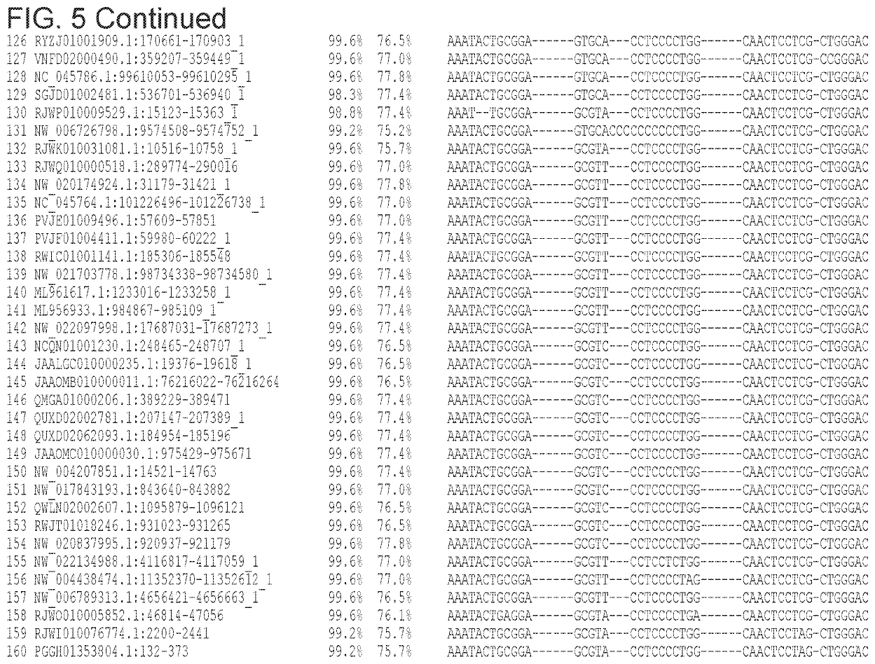

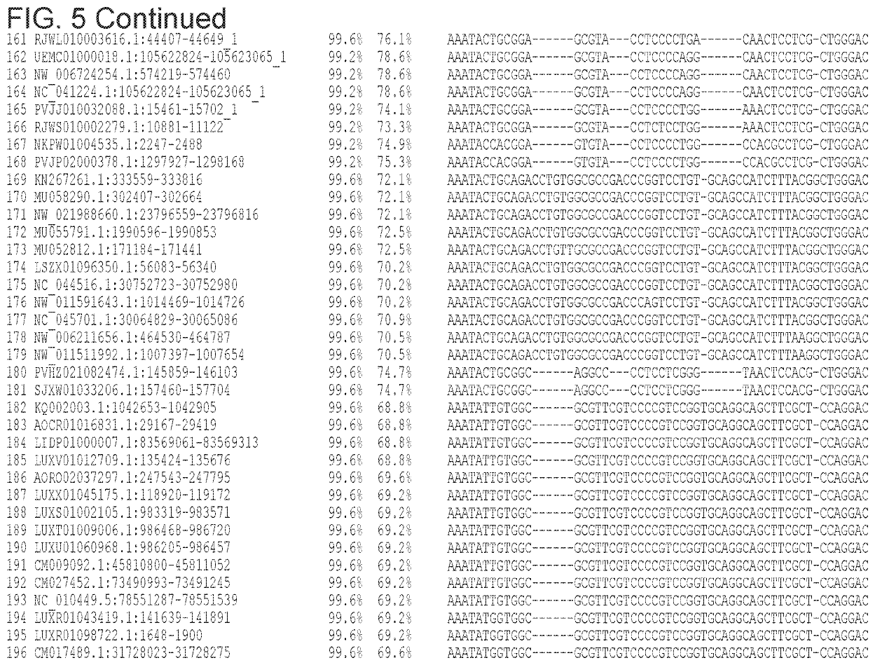

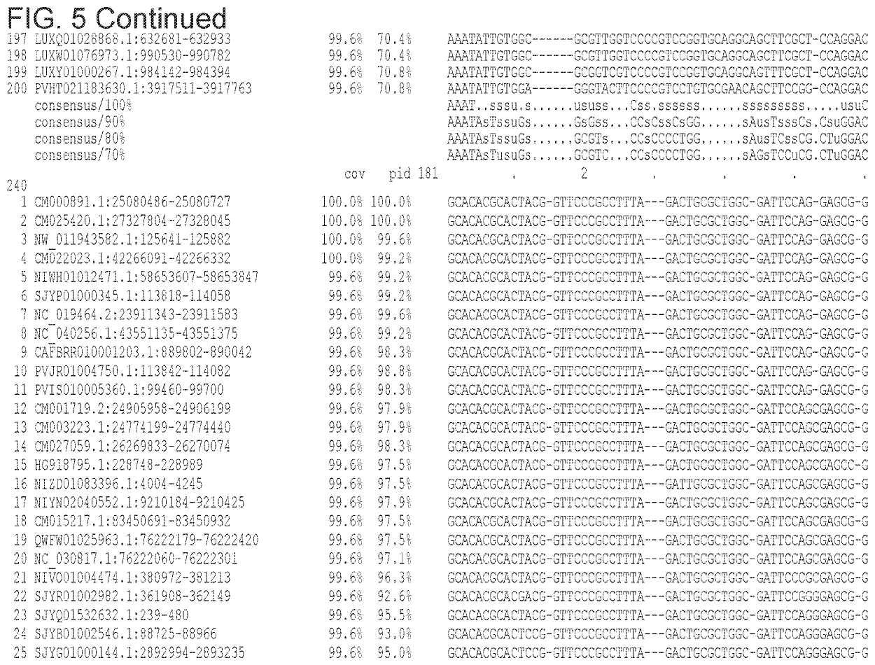

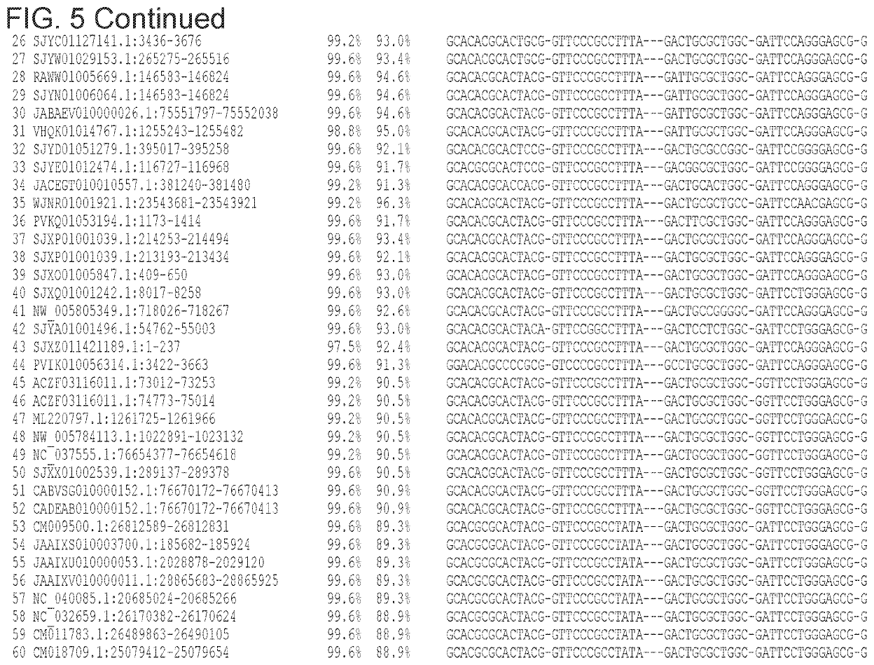

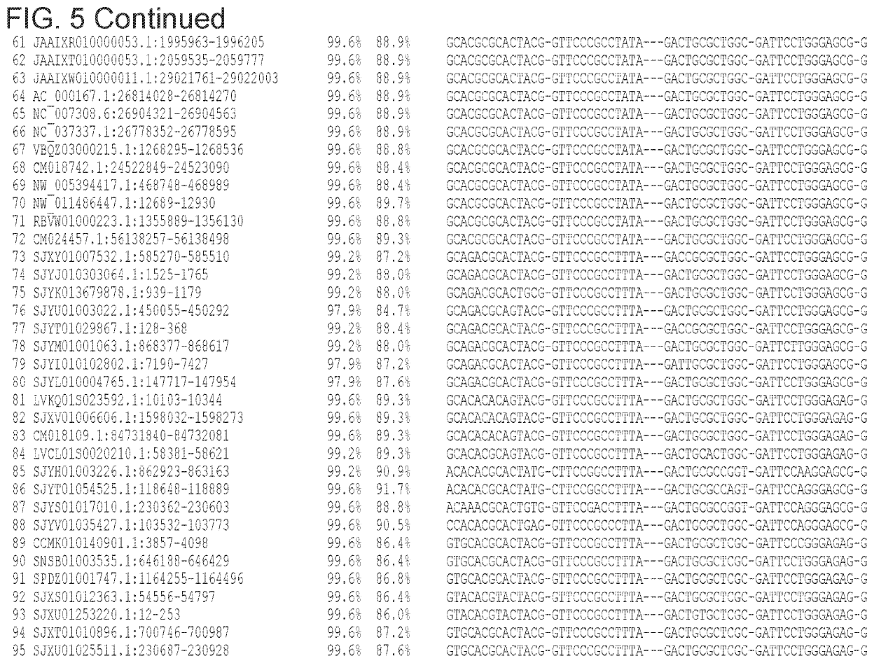

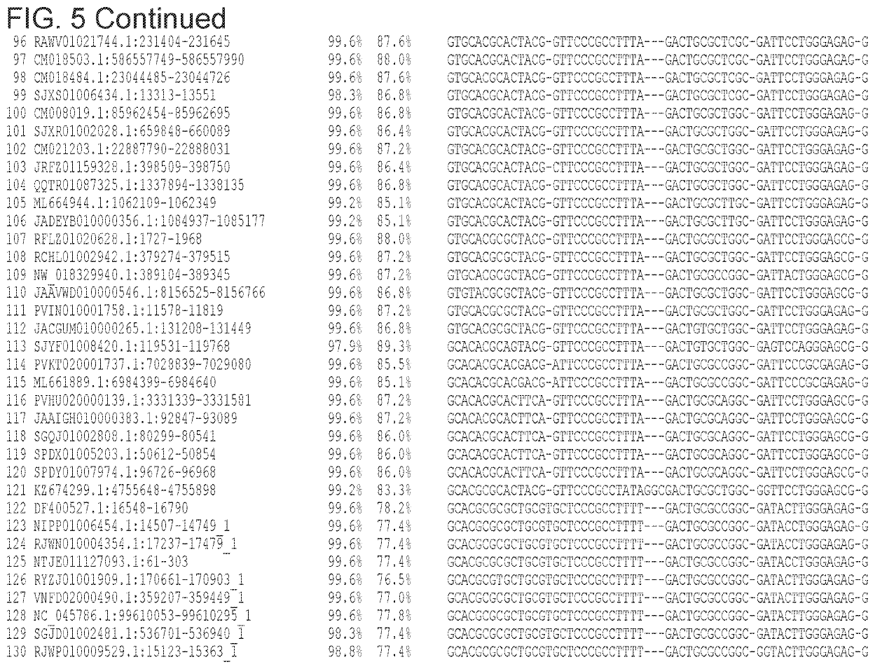

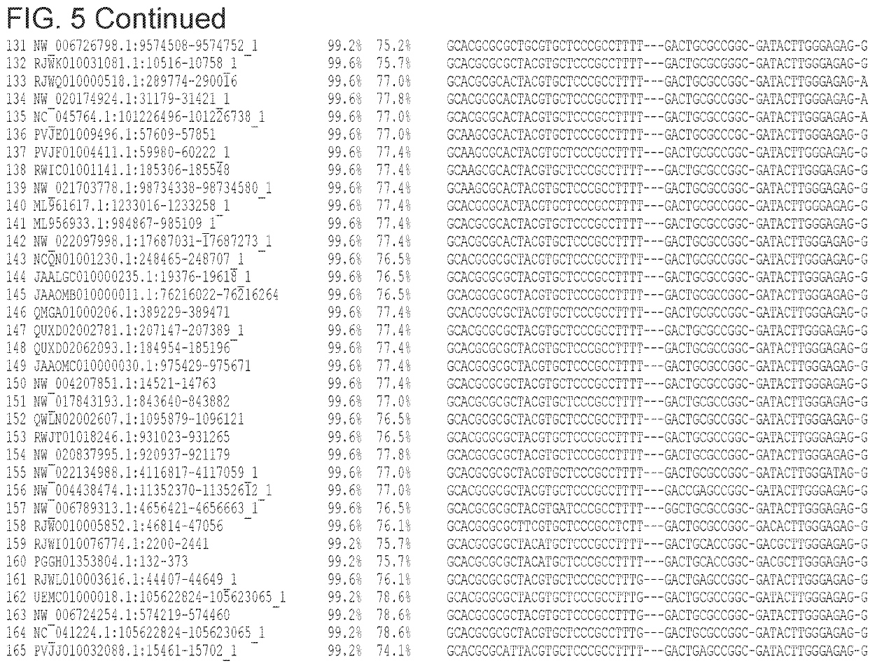

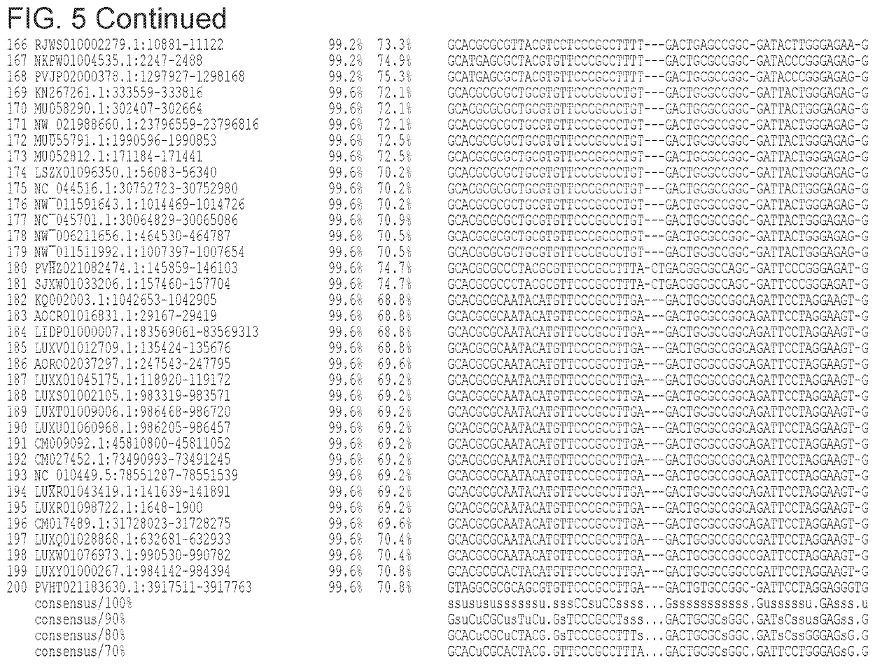

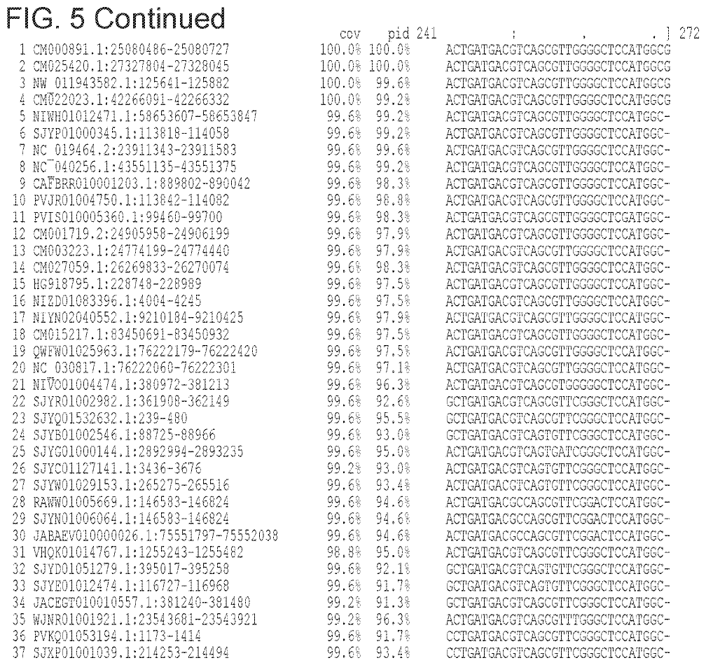

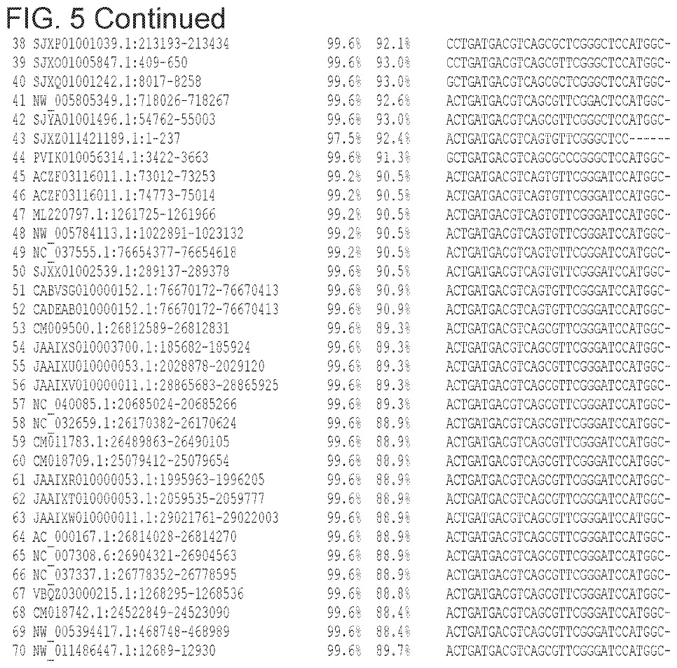

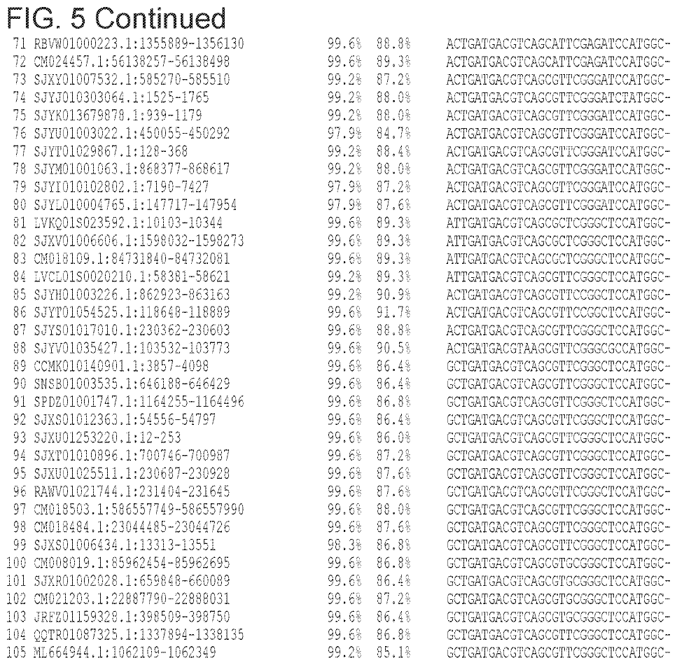

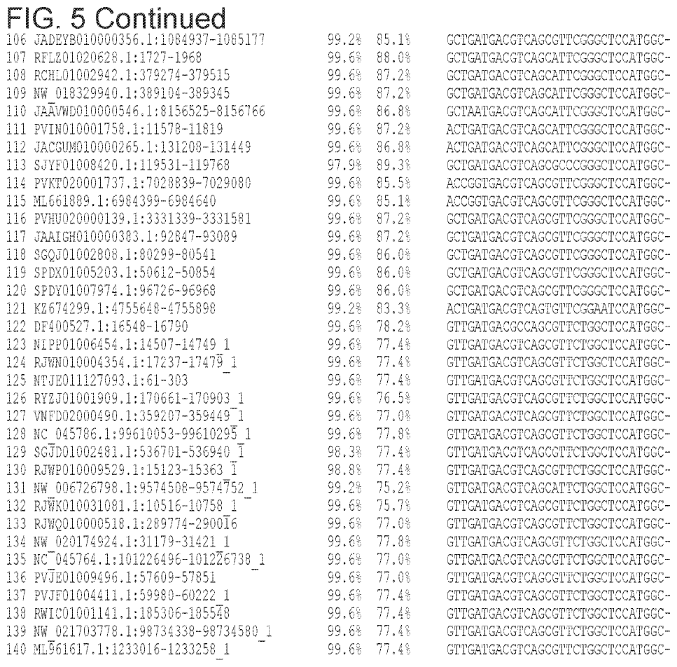

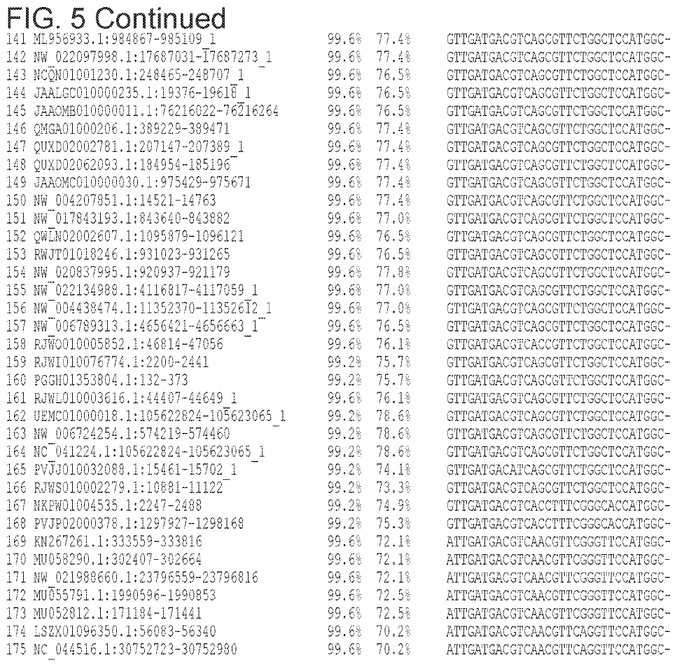

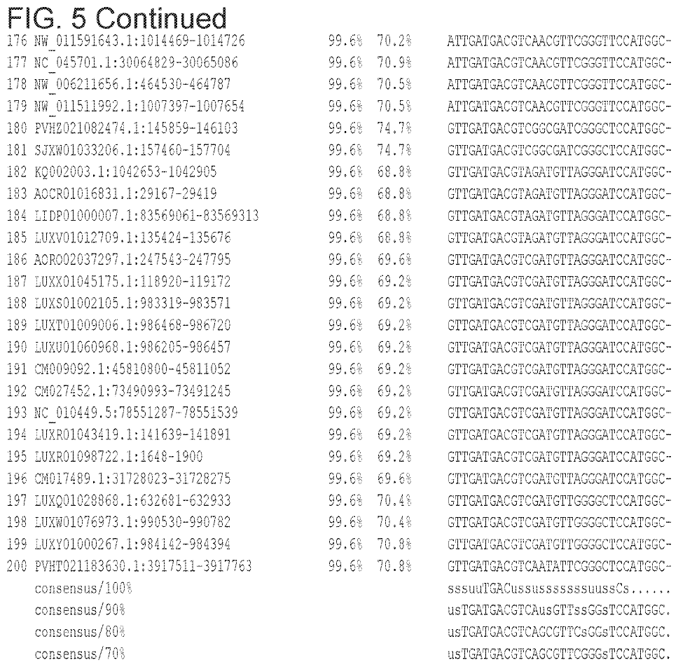

In certain embodiments, the promoter comprises an Artiodactyla H1 promoter. An alignment of Artiodactyla H1 promoter sequences is provided in FIG. 5 (wherein sequences numbered 1-200 in FIG. 5 correspond to SEQ ID NOs: 269-468 and consensus/100%, consensus/90%, consensus/80% and consensus/70% correspond to SEQ ID NOs 1811-1814, respectively). In some embodiments, the promoter comprises a nucleotide sequence having at least 85%. 90%, 95%. 96%, 97%. 98%. 99%, or 100% identity to nucleotides 20-266 of any one of the sequences in FIG. 5 or a functional fragment or variant (e.g., codon optimized) thereof.

In certain embodiments, the Artiodactyla H1 promoter comprises a sequence selected from the sequences in TABLE 5:

TABLE 5

| | |
|---|---|
| Artiodactyl | TGAGCTTCCCKCCGCCCTAYGSMRA |
| Alignment | AMAMYRSSCKCAARSMGCATTTATA |
| consensus | AKGMKCYCAWACCTARAGMCAYTTK |
| sequence | WCGGTTAYGGTGACTTCCCAYAASA |
| 75%_Identity | CATTGCGACATGCAAATAYTDYRGW |
| | GCGTYCCKCCCCTGGYARYTCCWCG |
| | CTRGGACGCACRCGCRCTACGNGTT |
| | CCCGCCTTTWGACTGCGCYGGCGAT |
| | TCCWGGGAGMGGRYTGATGACGTCA |
| | GCGTTCGGGMTCCATGGCG |
| | (SEQ ID NO: 469) |
| Artiodactyl | TGAGCTTCCCKCCGCCCTAYGBMRR |
| Alignment | AVRVYDSSYKCARDSMRCAYTTATA |
| consensus | ADGHKCYCADAMSTARAKMSAYTTB |
| sequence | WCRSTTAYGGTGACTTCYCRYAASA |
| 85%_Identity | CATTGSGAYATGCAAATAYTDYRGW |
| | GCGTYNNNCCKCSCCTGGNYARYTY |
| | YWCGCYRGGACGCACRCGCRCTRCG |
| | NGYTCCCGCCTTTWGACTGCGCYGG |
| | CGATWCYWGGGAGMGGRYTGATGAC |
| | GTCARYGTTSKGGMTCCATGGCG |
| | (SEQ ID NO: 470) |
| Artiodactyl | TGAGCTTCCCKCCGCCCYAYRBVRR |
| Alignment | ANRVYDVVYKCWRDBMRCRYTTATA |
| consensus | ANRHKCYCADAMSTARAKHSAYTTB |
| sequence | WYRSTTAYGGTGACTTCYCRYAASA |
| 90%_Identity | CAKTGSGRYATGCAAATAYTDYRGH |
| | GYGYHNNNCCBCSYCYGGNNNNNYA |
| | RYTYYDCKCYRGGACGYRCRCGCRM |
| | TRCRNGYTCCCGCCTWKWGACTGCG |
| | CYGGCGATWCYWRSGAGMKGRYTGA |
| | TGACGTCARYGTTSKGGMTCCATGG |
| | CG |
| | (SEQ ID NO: 471) |
| Artiodactyl | TGAGCTTCYCKCCGCCCYAYRNNRR |
| Alignment | RNRNBDVVBBCWVNBMRYVYTTATA |
| consensus | ANRHKCBCADAVBKARRKHVAYTTB |
| sequence | WYRVTTAYGGYGAYTTCYCNRHAMS |
| 95%_Identity | RCAKWGSRRYATGCAAATAYKDYRG |
| | HNNNNNNGYRYHNNNCCBSBYCYRK |
| | NNNNNNYADBTYYDCKNCYRGGACG |
| | YRSRCGCRMTRCRNGYTCCCGCCYW |
| | KWGACTGCGCYSGCNGATWMYHRNG |
| | ARVKGRYTGATGACGTCRRYRTTVK |
| | GGHTCCATGGCG |
| | (SEQ ID NO: 472) |
| Artiodactyl | TGAGCTTCYCDCCGCCCYRYVNNVR |
| Alignment | NNNNBNNNNNBDVNNHRYVYTTATA |
| consensus | ANRNDCBSRNRNBBNVRKNNAYNNN |
| sequence | HHRVTTAYGGYGAYTYCYCNRHAMS |
| 99% Identity | VMABWGSRRBATGYAAATAYBNYRG |
| | HNNNNNNRBRYHNNNCCBSBYCHDD |
| | NNNNNNHMDBKYYDHNNNNNGKACR |
| | YRNRCRYVVBNYRNSYTCCSGCCYW |
| | KDNNGAYBGHRCHVGYNGRYWMYNR |
| | NGARVKRVYTGATGACGYMRVYRHK |
| | VNGRHWCCATGGCG |
| | (SEQ ID NO: 473) |
| Artiodactyl | TGAGCTYCYCDCCGCCYYRHNNNNN |
| Alignment | NNNNNNNNNNBNNNNNNVNNNRYNN |
| consensus | TWATAWNRNDCBSRNVNNBNVRBNN |
| sequence | AYNNNHHVNYTAYGGYGAYTYCYCN |
| 100%_Identity | RHAMSVVABWGSRNRBATGYAAATN |
| | NBNHRNHNNNNNNRBRBHNNNCSNN |
| | BYYNDDNNNNNNNMDBBYBNNNNNN |
| | NRDRCVBRNRMRYVNNNHRNVHYCC |
| | SRCCYHKDNNNGVYBBHNSNNSYNG |
| | RBDMYNRNGADVNNRVYYRRTGACR |
| | YMRVYDHBNNRRHDCBATGGCG |
| | (SEQ ID NO: 474) |

In some embodiments, the promoter comprises a nucleotide sequence having at least 85%, 90%, 95%, 96%, 97%, 98%, 99%, or 100% identity to nucleotides 20-238 of any one of SEQ ID NOs: 469-474 or a functional fragment or variant (e.g., codon optimized) thereof.

### Carnivora H1 Promoters

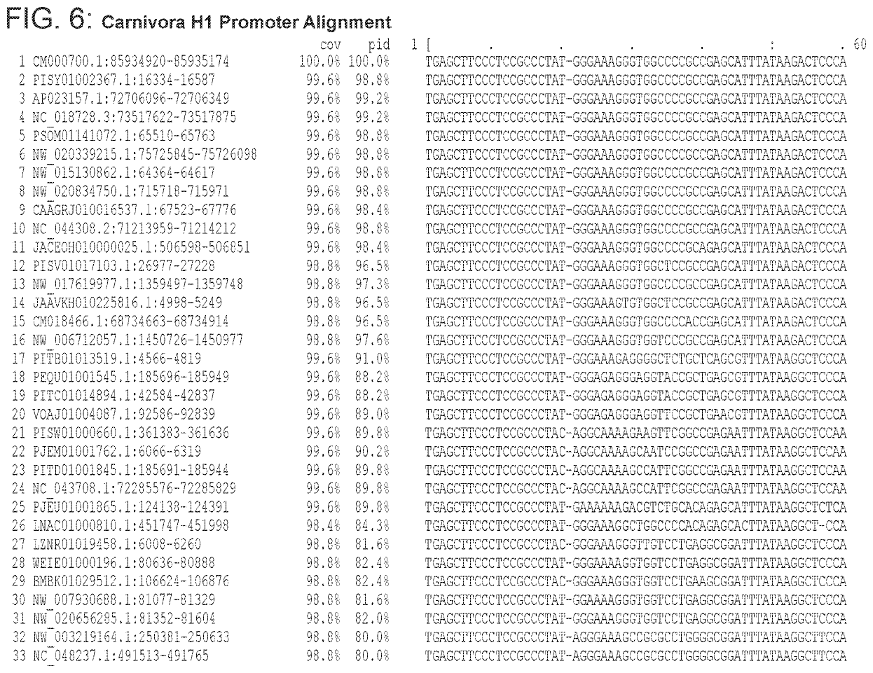

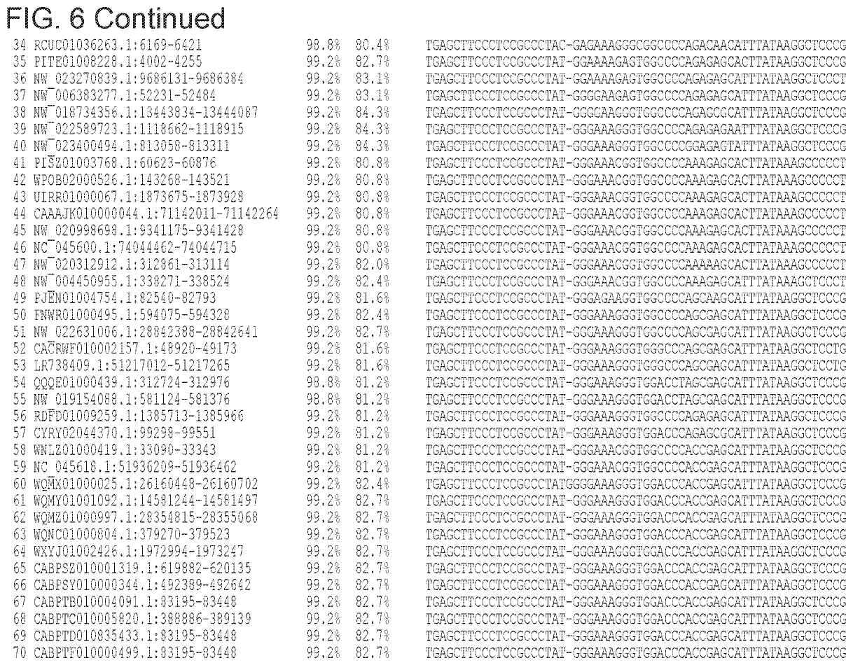

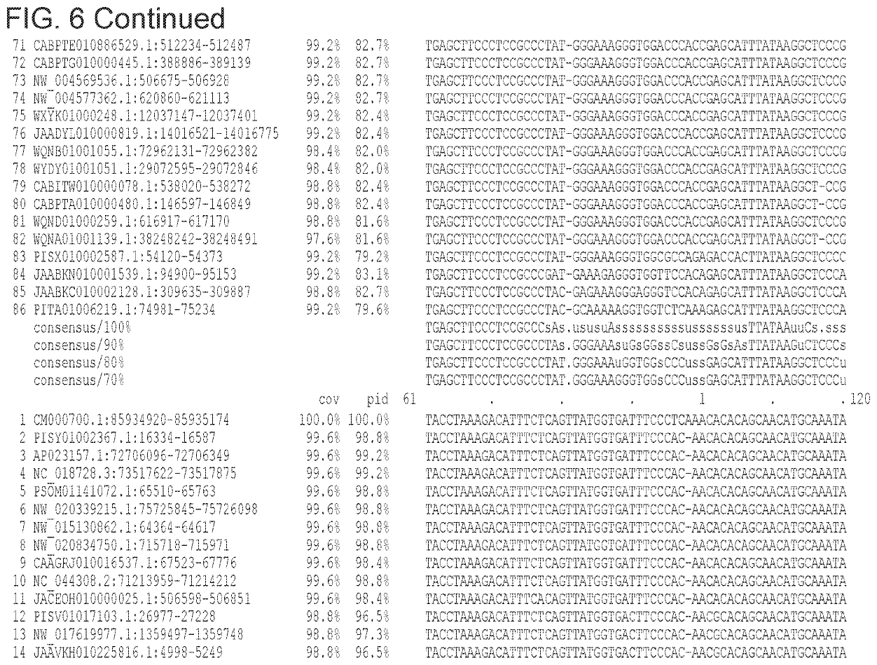

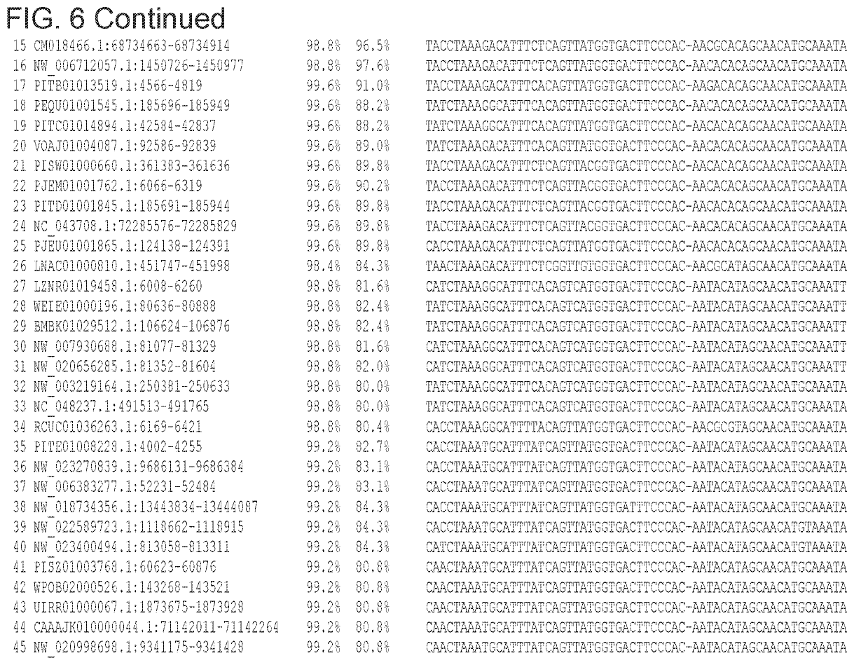

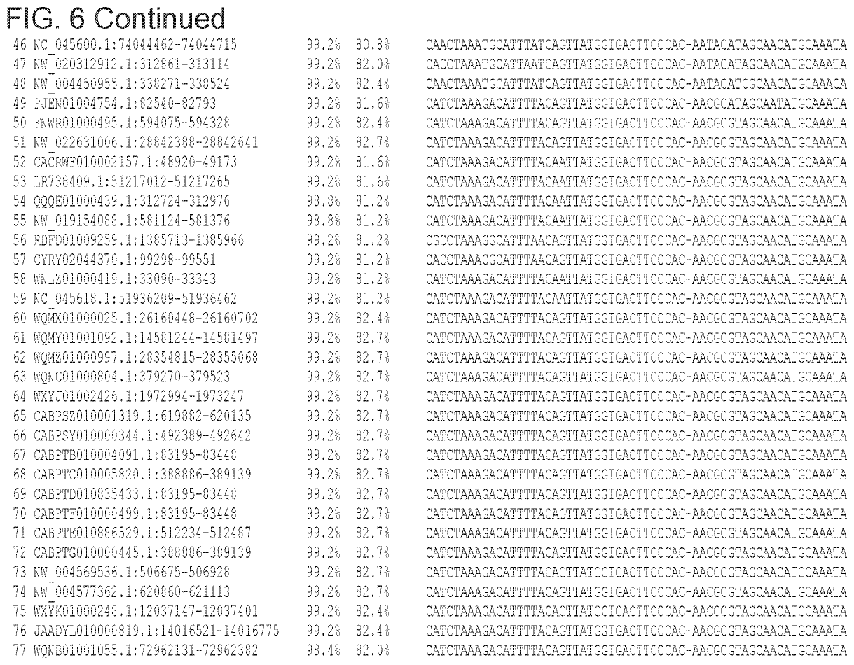

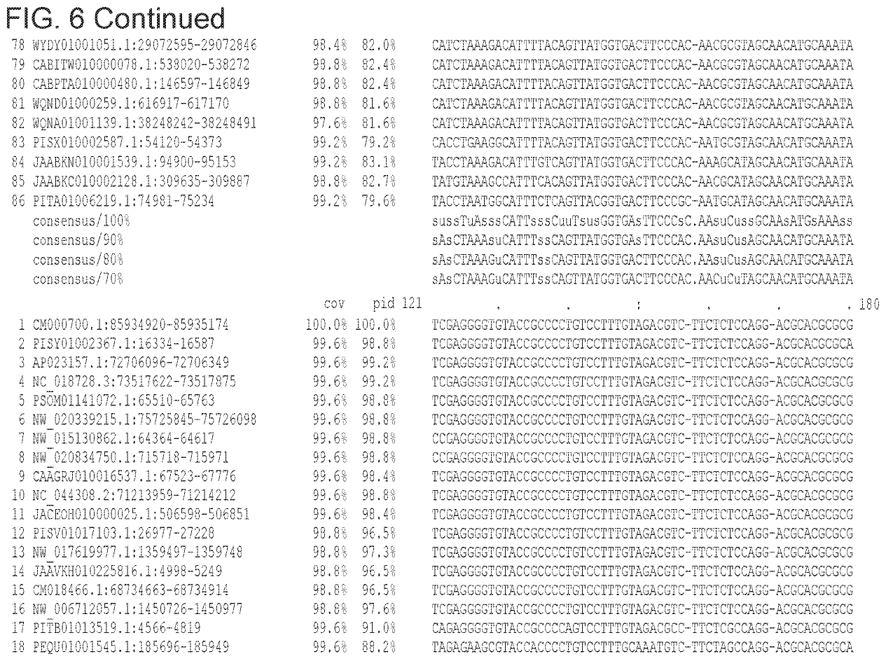

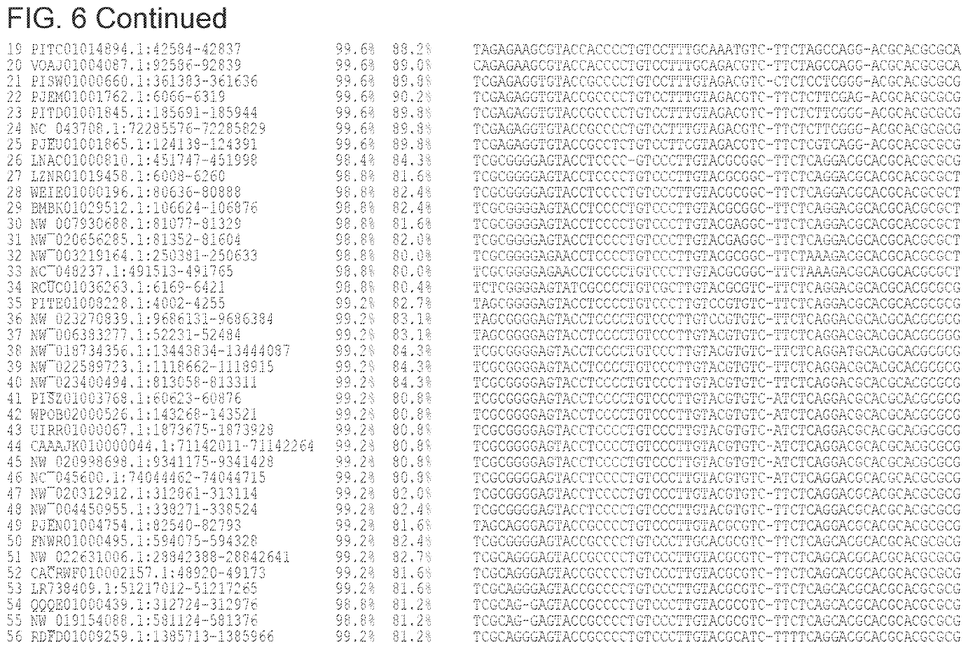

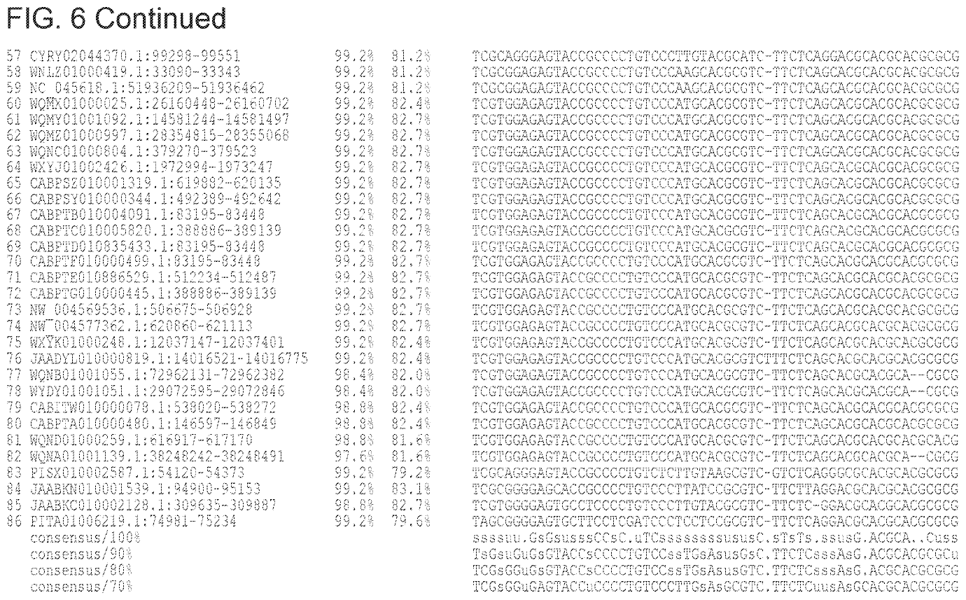

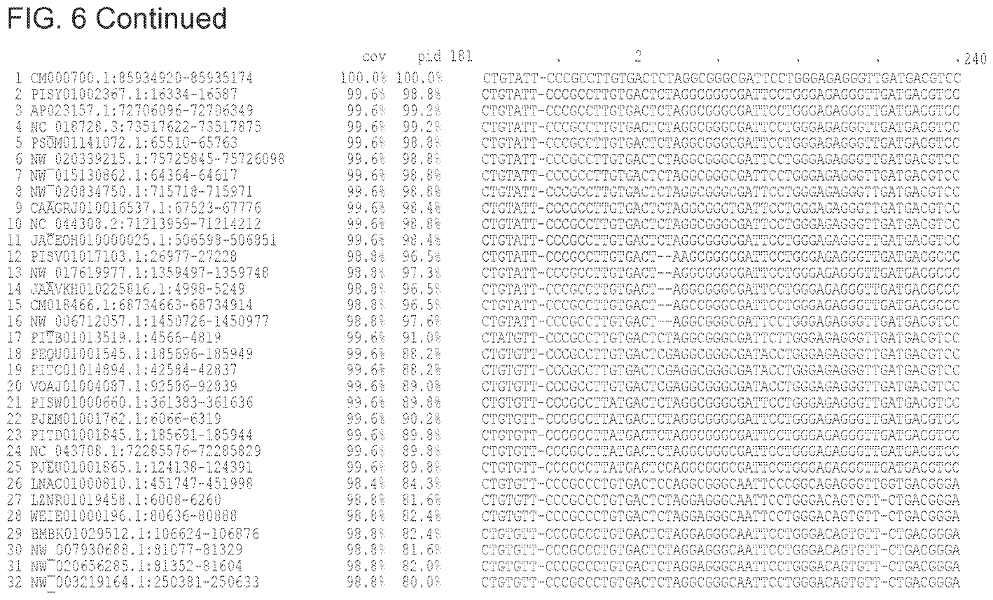

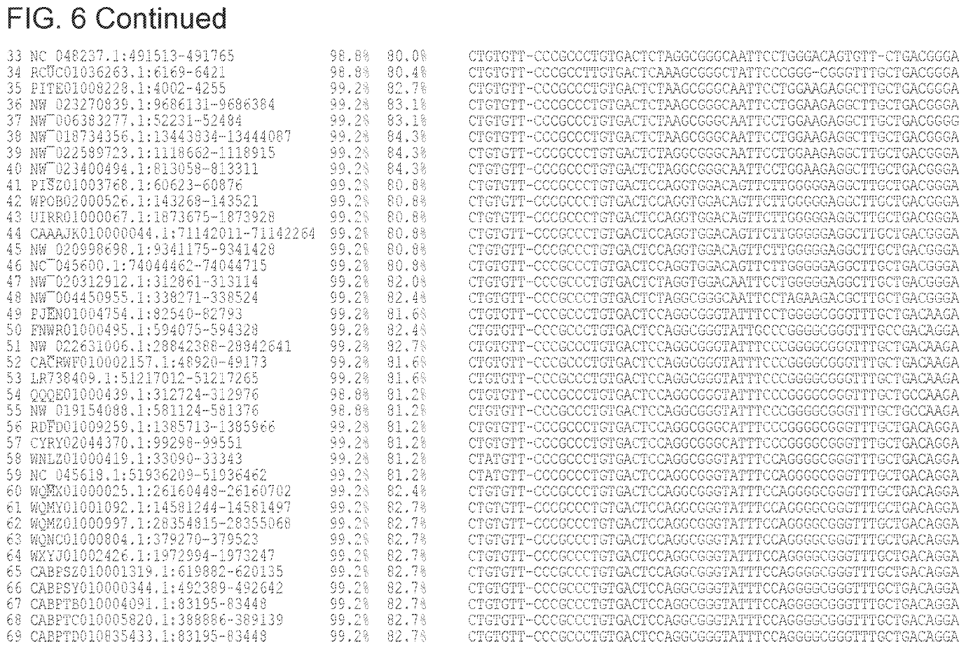

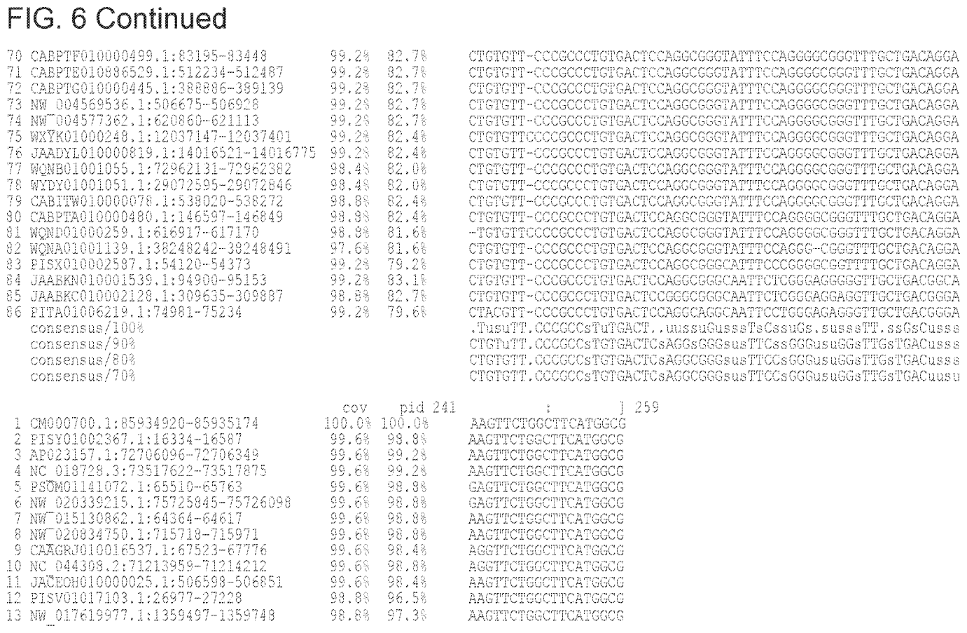

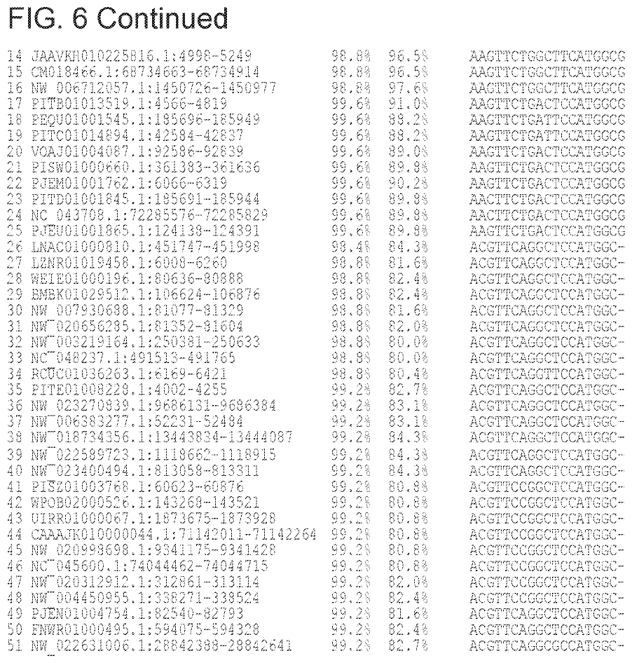

In certain embodiments, the promoter comprises a Carnivora H1 promoter. An alignment of Carnivora H1 promoter sequences is provided in FIG. 6 (wherein sequences numbered 1-86 in FIG. 6 correspond to SEQ ID NOs: 475-558 and SEQ ID NOs: 1809-1810, and consensus/100%, consensus/90%, consensus/80% and consensus/70% correspond to SEQ ID NOs: 1815-1818, respectively). In some embodiments, the promoter comprises a nucleotide sequence having at least 85%, 90%, 95%, 96%, 97%, 98%, 99%, or 100% identity to nucleotides 20 to 253 any one of the sequences in FIG. 6 or a functional fragment or variant (e.g., codon optimized) thereof.

In certain embodiments, the Carnivora H1 promoter comprises a sequence selected from those in TABLE 6.

TABLE 6

| | |
|---|---|
| Carnivora | TGAGCTTCCCTCCGCCCTATGGGGA |
| Alignment | AAGGGTGGMCCCRSMGAGCATTTAT |
| consensus | AAGGCTCCCRYAYCTAAAGRCATTT |
| sequence | YWCAGTTATGGTGACTTCCCACAAA |
| 75%_Identity | YRCRYAGCAACATGCAAATATCGHG |
| | GRGWGTACCKCCCCTGTCCYWTGYA |
| | SRCGTCTTTCTCWSSASGCACGCAC |
| | GCGCGCTGTGTTCCCCGCCYTGTGA |
| | CTCYAGGCGGGYRWTTCCWGGGRSR |
| | GGKTTGMTGACRKSMAMGTTCWGGC |
| | TYCATGGCG (SEQ ID NO: 559) |
| Carnivora | TGAGCTTCCCTCCGCCCTATGGGGA |
| Alignment | AAVGGYGGHYCYRVMGAGSATTTAT |
| consensus | AAGRCTCCCRYAYCTAAAKRCATTT |
| sequence | HWCAGTTATGGTGACTTCCCACAAA |
| 85%_Identity | YRCRYAGCAACATGCAAATATCGHG |
| | GRGWGTACCKCCCCTGTCCYWTGYA |
| | SRYGTCTTTCTCWSSASGCACGCAC |
| | GCGCGCTGTRTTCCCCGCCYTGTGA |
| | CTCYAGGCGGGYRWTTCCHGGGRSR |
| | GGBTTGMTGACRKSMAMGTTCWGGC |
| | TYCATGGCG (SEQ ID NO: 560) |
| Carnivora | TGAGCTTCCCTCCGCCCTAYGGGGA |
| Alignment | AAVRGYGGHYCYRVVGMGSAYTTAT |
| consensus | AAGRCTCCCDYAYCTAAAKRCATTT |
| sequence | HWCAGTTATGGTGAYTTCCCACAAA |
| 90% Identity | YRCRYAGCAACATGCAAATATMGHR |
| | GRGWGTACCKCCCCTGTCCYWTGYA |
| | SRYGKCTTTCTCWSSASGCACGCAC |
| | GCGCKCTGTRTTCCCCGCCYTGTGA |
| | CTCYAGGYGGGYRWTTCYHGGGRSR |
| | GGBTTGMTGACRDSMAMGTTCWGRC |
| | TYCATGGCG (SEQ ID NO: 561) |
| Carnivora | TGAGCTTCCCTCCGCCCTAYGRRRV |
| Alignment | RAVRGHVRNYCYRVVGMGVAYTTAT |
| consensus | AARRCYCCMDYAHCTAAAKRCATTT |
| sequence | HWCARTYAYGGTGAYTTCCCACAAA |
| 95%_Identity | YRCRYAGCAACATGCAAATWTMGHR |
| | RRGWGTACCKCCCCTGTCCYWTGYA |
| | SRYGKCTWTCTMDBSRSGCACGCAC |
| | GCGCKCTGTRTTCCCCGCCYTRTGA |
| | CTCYARGHGGRYRDTTCYHGGRRSR |
| | GKBTTGMTGACRDSMAMGTTCHGRC |
| | TYCATGGCG (SEQ ID NO: 562) |
| Carnivora | TGAGCTTCCCTCCGCCCKAYGRVRV |
| Alignment | RAVDVNNNNNBBRVNVMVNRYTTAT |
| consensus | AARRCYYYHNYRHSTRAWBVCATTW |
| sequence | NWCRRTYRYGGTGAYTTCCCDCAAA |
| 99%_Identity | NRCRYMGCAAYATGYAAAYWYMKHR |
| | RRGHGHRYYDCCYCDRTCBYWHVYM |
| | VRHRBCTNTYTHNNSRNGCACGCAC |
| | GCRSDCTRYRTTCCCCGCCYTRTGA |
| | CTCNRRSHRGRYDDTDCYHRGVRSR |
| | VKBTTGVYGMCRNSVRVBTYCHGRY |
| | KYCATGGCG (SEQ ID NO: 563) |
| Carnivora | TGAGCTTCCCTCCGCCCKAYGRVRV |
| Alignment | RAVDVNNNNNBBRVNVMVNRYTTAT |
| consensus | AARRCYYYHNYRHSTRAWBVCATTW |
| sequence | NWCRRTYRYGGTGAYTTCCCDCAAA |
| 100%_Identity | NRCRYMGCAAYATGYAAAYWYMKHR |
| | RRGHGHRYYDCCYCDRTCBYWHVYM |
| | VRHRBCTNTYTHNNSRNGCACGCAC |
| | GCRSDCTRYRTTCCCCGCCYTRTGA |
| | CTCNRRSHRGRYDDTDCYHRGVRSR |
| | VKBTTGVYGMCRNSVRVBTYCHGRY |
| | KYCATGGCG (SEQ ID NO: 564) |

In some embodiments, the promoter comprises a nucleotide sequence having at least 85%, 90%, 95%, 96%, 97%, 98%, 99%, or 100% identity to nucleotides 20-253 of any one of SEQ ID NOs: 559-564 or a functional fragment or variant (e.g., codon optimized) thereof.

### Cetacea H1 Promoters

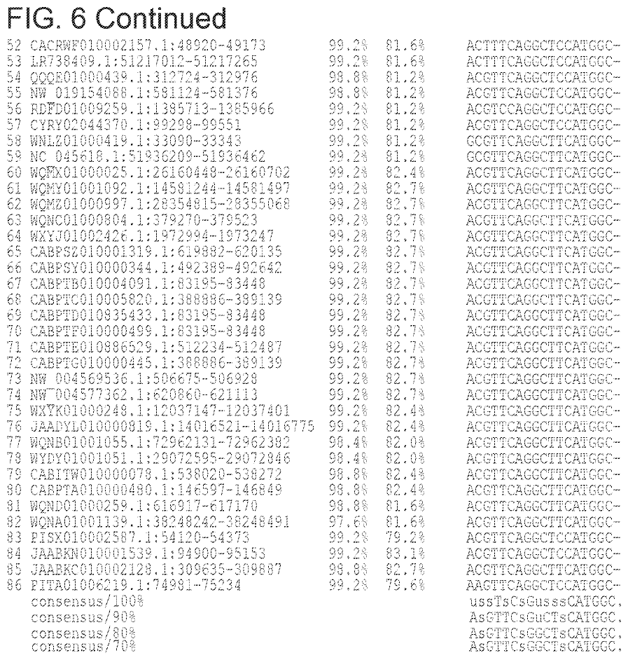

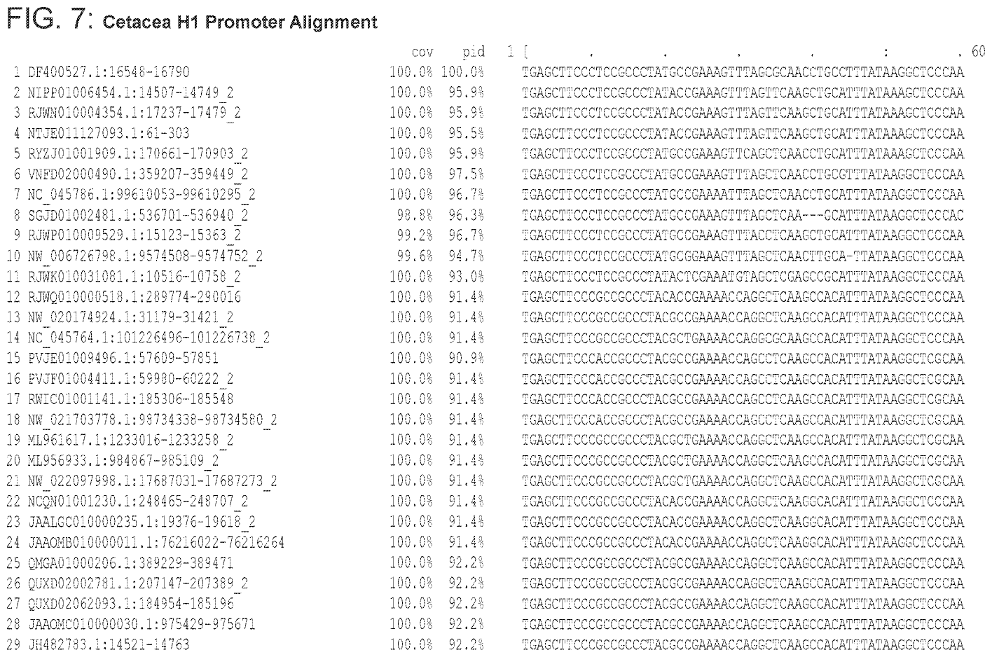

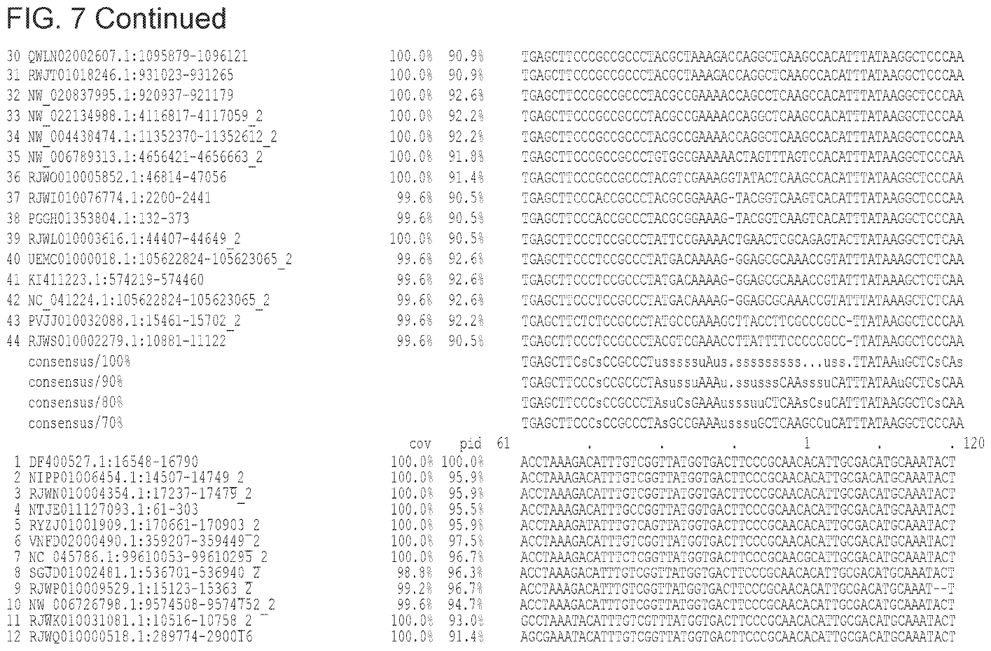

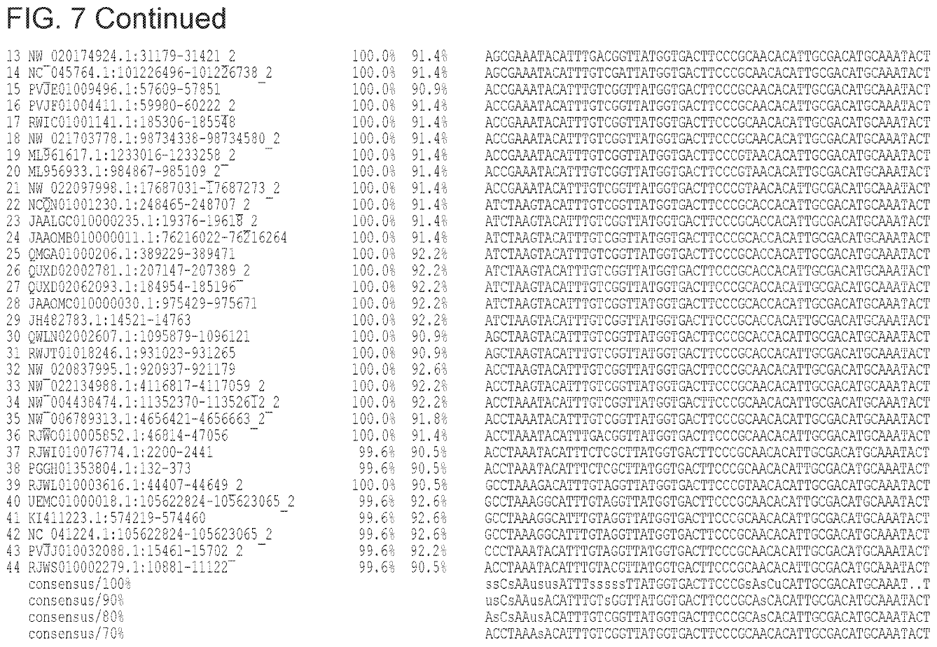

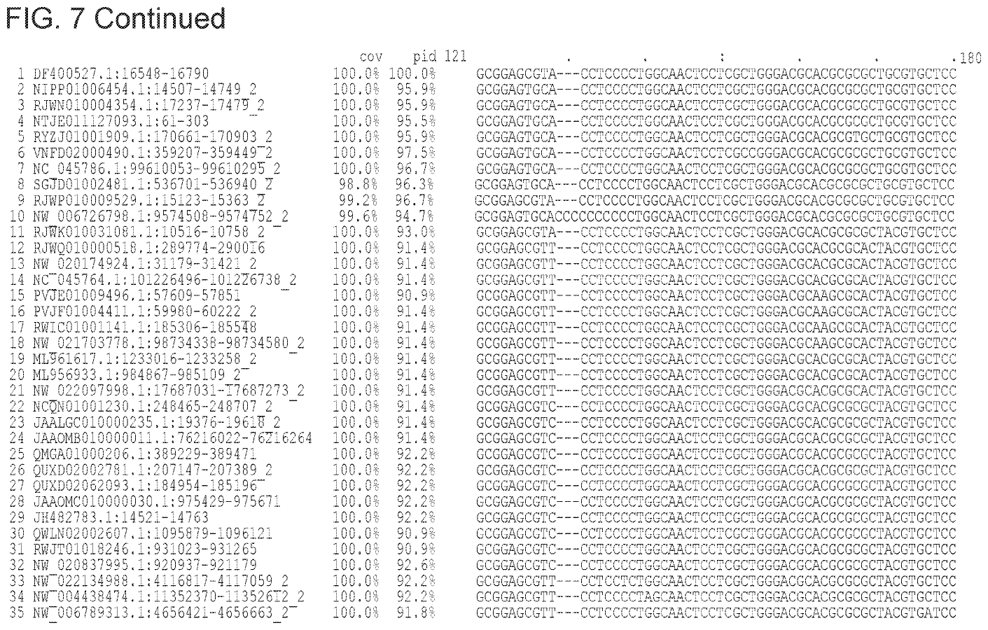

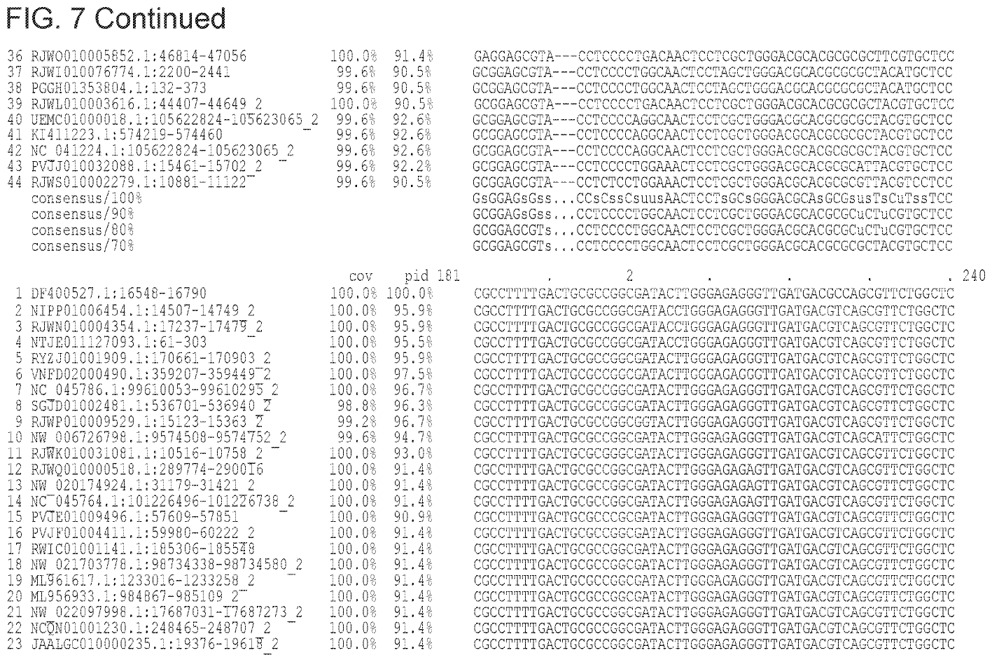

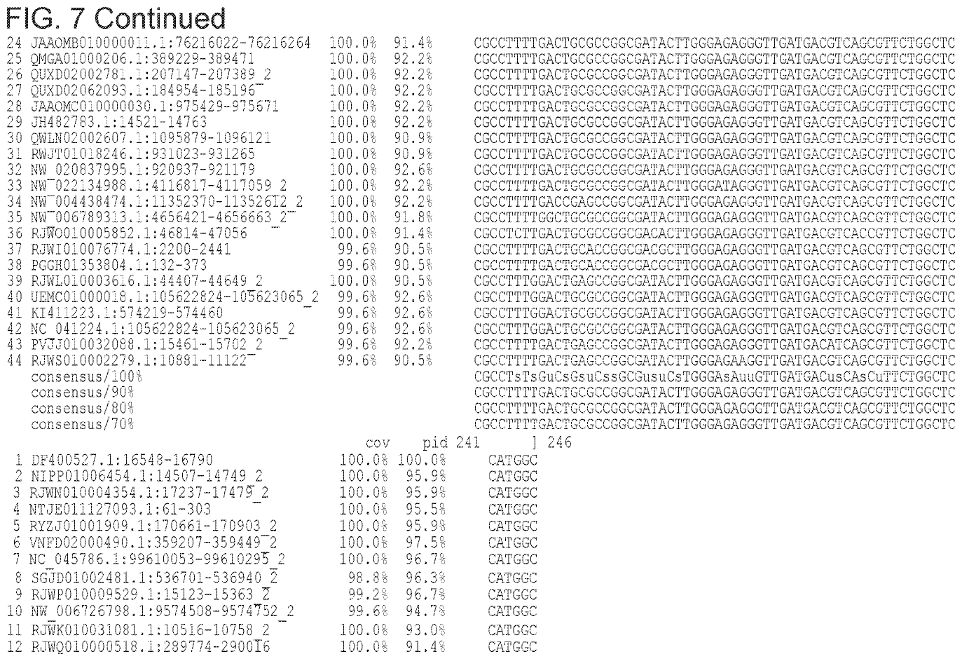

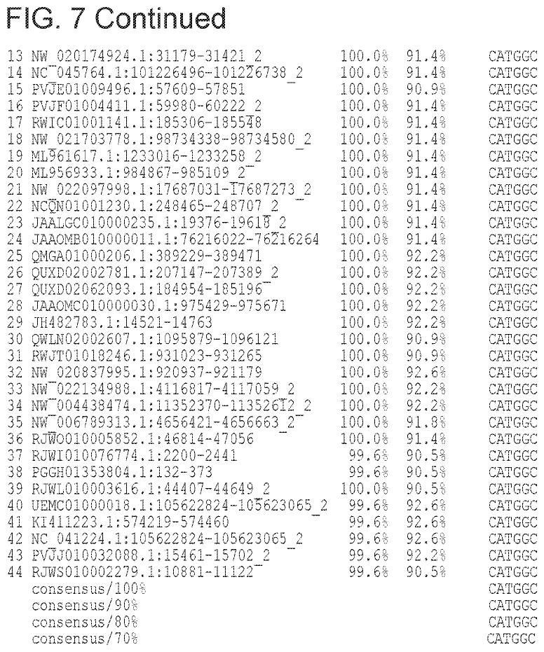

In certain embodiments, the promoter comprises a Cetacea H1 promoter. An alignment of Cetacea H1 promoter sequences is provided in FIG. 7 (wherein sequences numbered 1-44 in FIG. 7 correspond to SEQ ID NOs: 565-608, and consensus/100%, consensus/90%, consensus/80% and consensus/70% correspond to SEQ ID NOs: 1819-1822, respectively). In some embodiments, the promoter comprises a nucleotide sequence having at least 85%, 90%, 95%, 96%, 97%, 98%, 99%, or 100% identity to nucleotides 20-241 of any one of the sequences in FIG. 7 or a functional fragment or variant (e.g., codon optimized) thereof.

In certain embodiments, the Cetacea H1 promoter comprises a sequence selected from those in TABLE 7.

TABLE 7

| | |
|---|---|
| Cetacea | TGAGCTTCCCKCCGCCCTAYGCCGA |
| Alignment | AARYYWRGCTCAASCCRCATTTATA |
| consensus | AGGCTCCCAAAYCTAARKACATTTG |
| sequence | TCGGTTATGGTGACTTCCCGCAACA |
| 75%_Identity | CATTGCGACATGCAAATACTGCGGA |
| | GCGTWCCTCCCCTGGCAACTCCTCG |
| | CTGGGACGCACGCGCGCTACGTGCT |
| | CCCGCCTTTTGACTGCGCCGGCGAT |
| | ACTTGGGAGAGGGTTGATGACGTCA |
| | GCGTTCTGGCTCCATGGC |
| | (SEQ ID NO: 609) |
| Cetacea | TGAGCTTCCCKCCGCCCTAYRCYGA |
| Alignment | AARNYWRSYTCAASSYRCATTTATA |
| consensus | ARGCTCSCAAAYCKAARKACATTTG |
| sequence | TCGGTTATGGTGACTTCCCGCAMCA |
| 85%_Identity | CATTGCGACATGCAAATACTGCGGA |
| | GYGYHCCTCCCCTGGCAACTCCTCG |
| | CTGGGACGCACGCGCRCTRCGTGCT |
| | CCCGCCTTTTGACTGCGCCGGCGAT |
| | ACTTGGGAGAGGGTTGATGACGTCA |
| | GCGTTCTGGCTCCATGGC |
| | (SEQ ID NO: 610) |
| Cetacea | TGAGCTTCCCDCCGCCCTAYRMYRA |
| Alignment | AARNYDRSYKCAAVSYRCATTTATA |
| consensus | ARGCTCSCAARBCKAARKACATTTG |
| sequence | TMGGTTATGGTGACTTCCCGCAMCA |
| 90%_Identity | CATTGCGACATGCAAATACTGCGGA |
| | GYGYHCCTCCCCTGGCAACTCCTCG |
| | CTGGGACGCACGCGCRCTRCGTGCT |
| | CCCGCCTTTTGACTGCGCCGGCGAT |
| | ACTTGGGAGAGGGTTGATGACGTCA |
| | GCGTTCTGGCTCCATGGC |
| | (SEQ ID NO: 611) |
| Cetacea | TGAGCTTCCCDCCGCCCTAYRHBRA |
| Alignment | AARNBDVVYKYVVVBYRYMNTTATA |
| consensus | ARGCTCBCAARBCKAARKRCATTTS |
| sequence | WMGSTTATGGTGACTTCCCGYAMCA |
| 95%_Identity | CATTGCGACATGCAAATACTGCGGA |
| | GYGYHCCTCCCCWGGCAACTCCTCG |
| | CTGGGACGCAMGCGCRCTRCGTGCT |
| | CCCGCCTTTKGACTGMGCCGGCGAY |
| | ACYTGGGAGAGRGTTGATGACGTCA |
| | GCGTTCTGGCTCCATGGC |
| | (SEQ ID NO: 612) |
| Cetacea | TGAGCTTCYCDCCGCCCTRYDNBVR |
| Alignment | ARVNBNNNBKYVVNNNRYVNTTATA |
| consensus | ARGCTCBCAMVBCKAARKRYATTTS |
| sequence | HMVNTTATGGTGACTTCCCGYAMCR |
| 99%_Identity | CATTGCGACATGCAAATNNTGMGGA |
| | GYGYHNNNCCYCYYCWRRMAACTCC |
| | TMGCYGGGACGCAMGCGYRYTDCRT |
| | SMTCCCGCCTYTKGRCYGMRCSSGC |
| | GRYRCYTGGGAKARRGTTGATGACR |
| | YCASCRTTCTGGCTCCATGGC |
| | (SEQ ID NO: 613) |
| Cetacea | TGAGCTTCYCDCCGCCCTRYDNBVR |
| Alignment | ARVNBNNNBKYVVNNNRYVNTTATA |
| consensus | ARGCTCBCAMVBCKAARKRYATTTS |
| sequence | HMVNTTATGGTGACTTCCCGYAMCR |
| 100%_Identity | CATTGCGACATGCAAATNNTGMGGA |
| | GYGYHNNNCCYCYYCWRRMAACTCC |
| | TMGCYGGGACGCAMGCGYRYTDCRT |
| | SMTCCCGCCTYTKGRCYGMRCSSGC |
| | GRYRCYTGGGAKARRGTTGATGACR |
| | YCASCRTTCTGGCTCCATGGC |
| | (SEQ ID NO: 614) |

In some embodiments, the promoter comprises a nucleotide sequence having at least 85%, 90%, 95%, 96%, 97%, 98%, 99%, or 100% identity to nucleotides 20-238 of any one of SEQ ID NOs: 609-614 or a functional fragment or variant (e.g., codon optimized) thereof.

### Chiroptera H1 Promoters

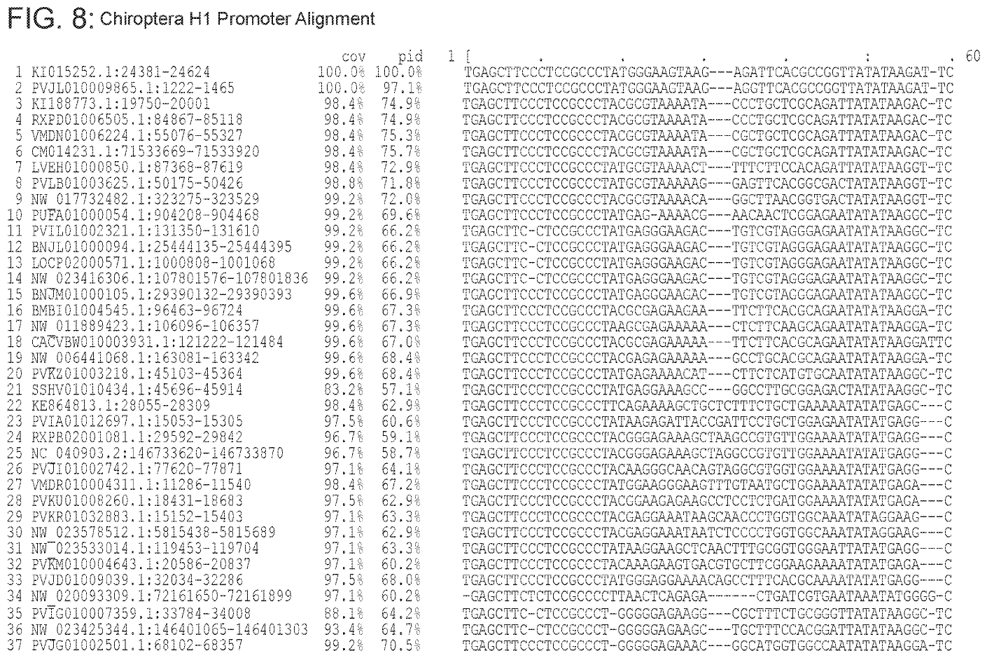

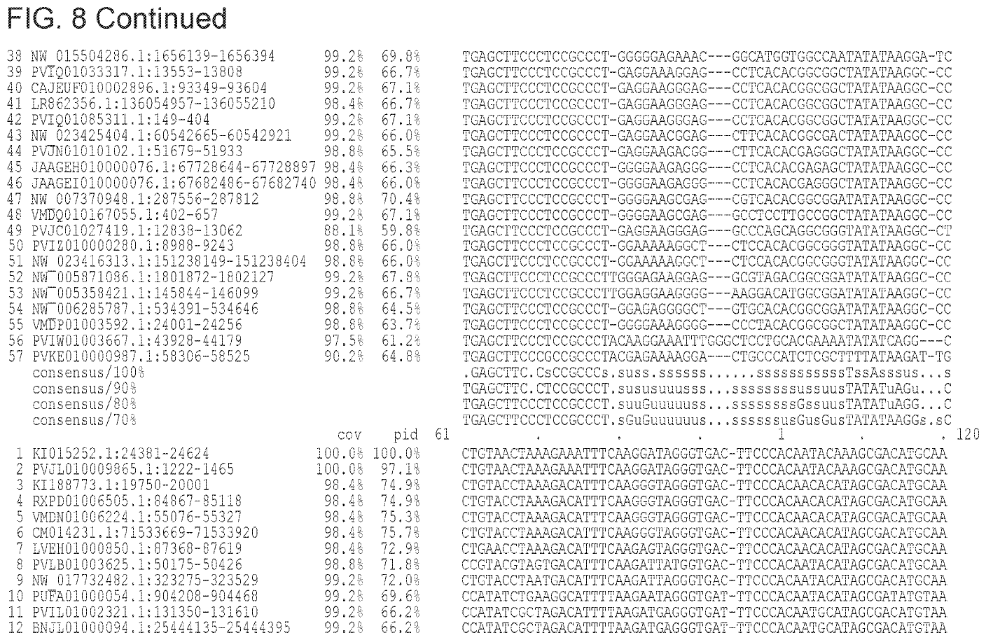

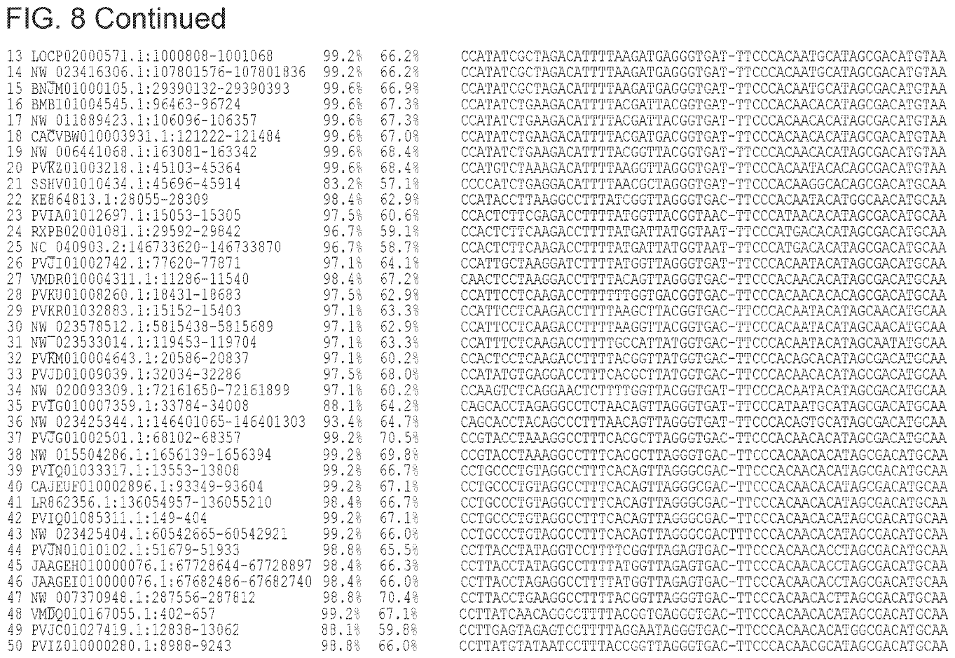

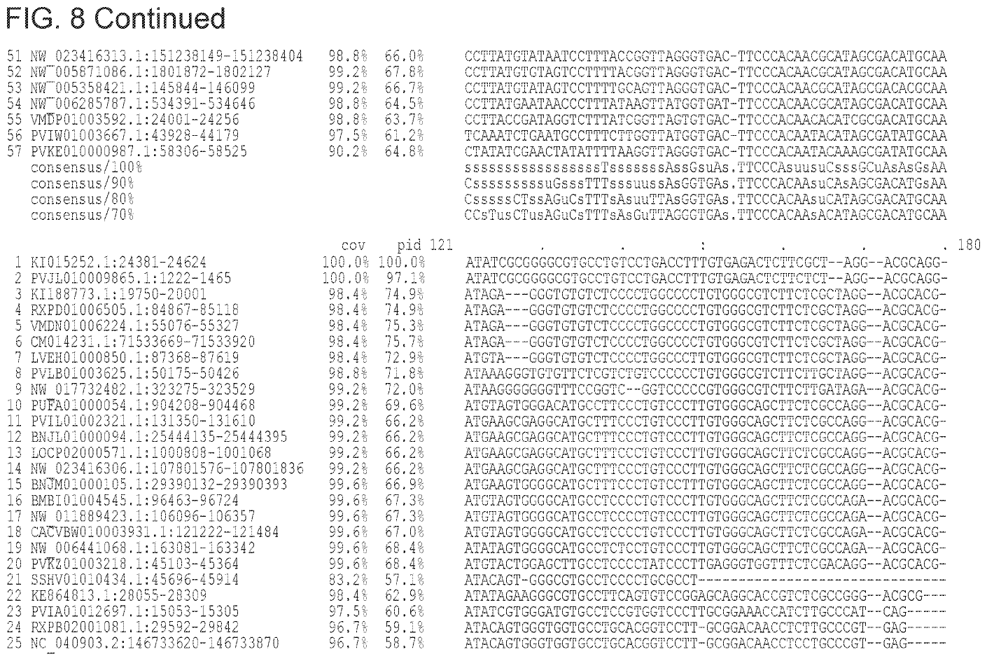

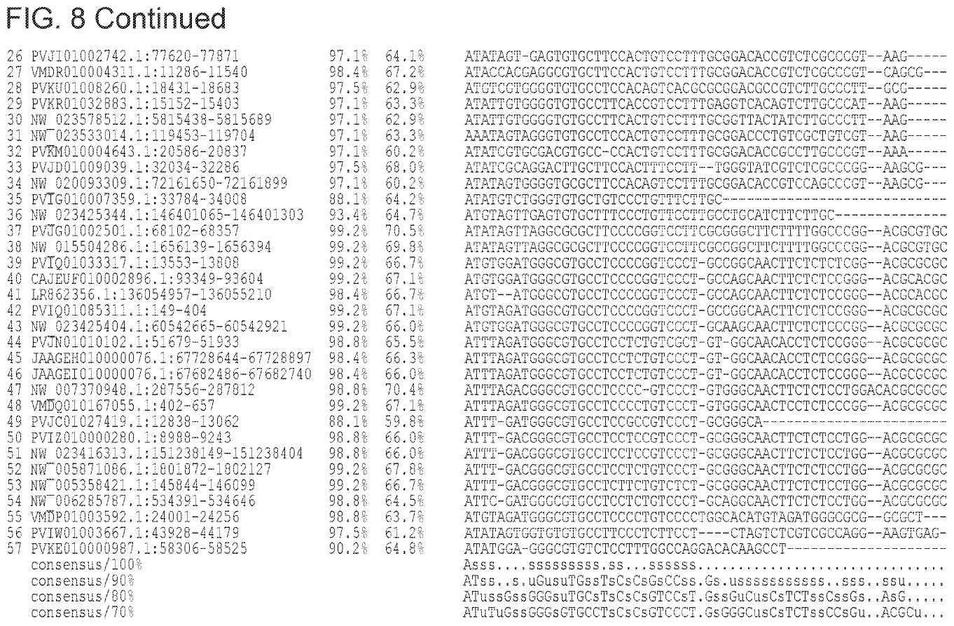

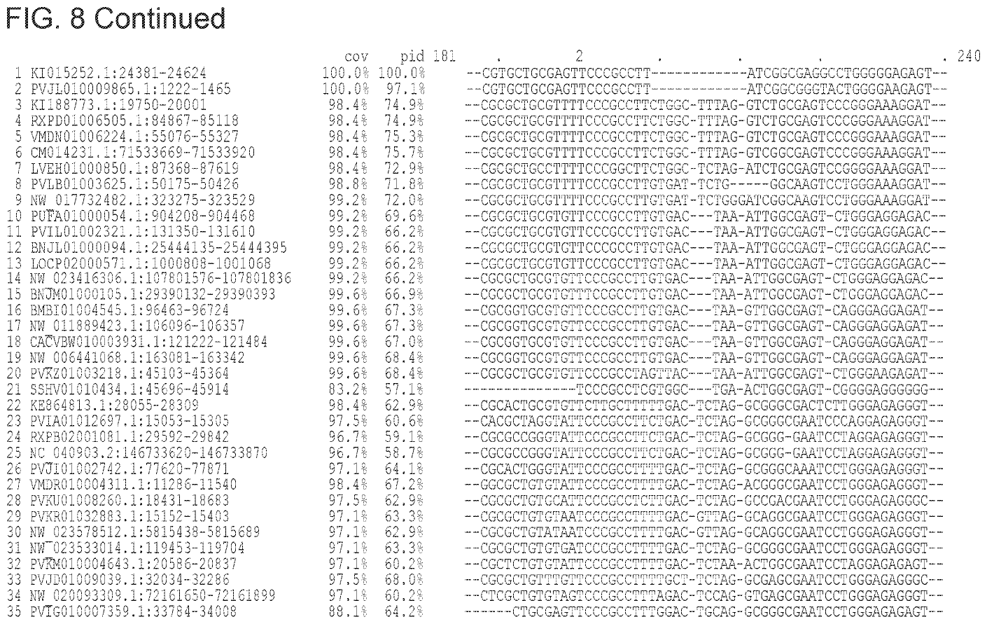

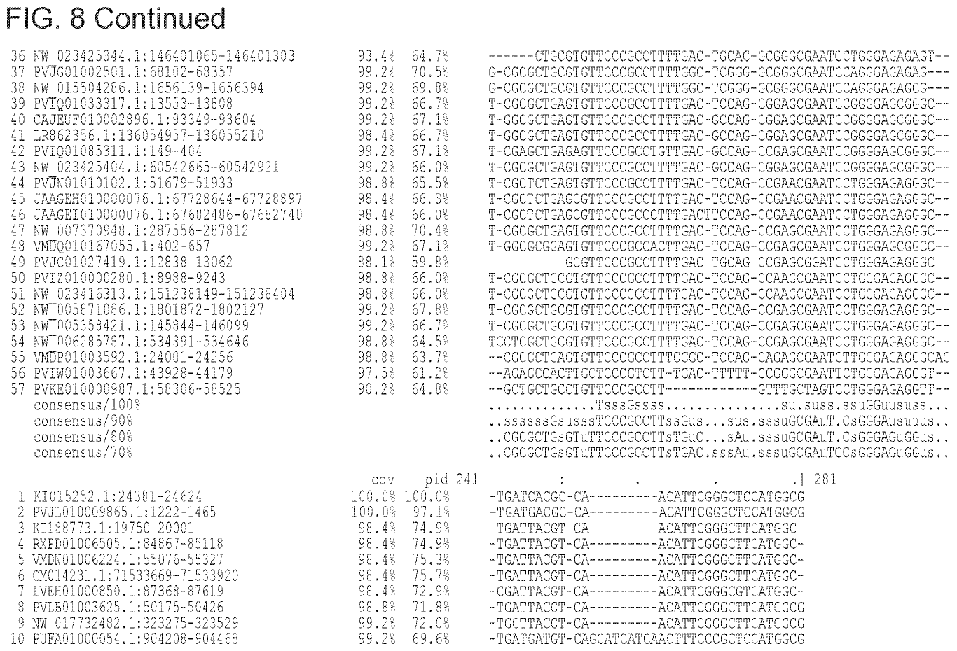

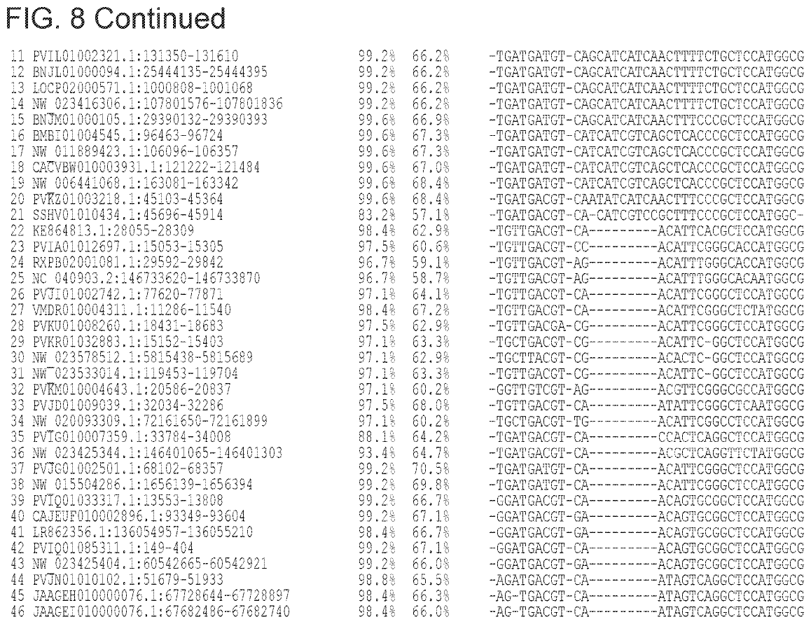

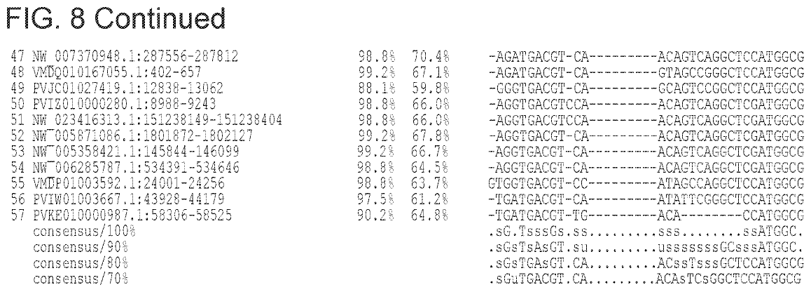

In certain embodiments, the promoter comprises a Chiroptera H1 promoter. An alignment of Chiroptera H1 promoter sequences is provided in FIG. 8 (wherein sequences numbered 1-57 in FIG. 8 correspond to SEQ ID NOs: 615-671, and consensus/100%, consensus/90%, consensus/80% and consensus/70% correspond to SEQ ID NOs: 1823-1826, respectively). In some embodiments, the promoter comprises a nucleotide sequence having at least 85%, 90%, 95%, 96%, 97%, 98%, 99%, or 100% identity to nucleotides 20-276 of any one of the sequences in FIG. 8 or a functional fragment or variant (e.g., codon optimized) thereof.

In certain embodiments, the Chiroptera H1 promoter comprises a sequence selected from those in TABLE 8.

TABLE 8

| | |
|---|---|
| Chiroptera | TGAGCTTCCCTCCGCCCTNBGRGRR |
| Alignment | RRRVVBBYYWSNYGSMRRMTATATA |
| consensus | AGGNYCCCWYWYCTVWAGRCMTTTY |
| sequence | AMGRTTASGGTGAYTTCCCACAAYA |
| 75% Identity | CATAGCGACATGCAAATRWNGHNGG |
| | GYGTGCCTYCMCKGTCCYTNGYSGR |
| | CRDCKTCTYKCYVGKAMGNNNNNNC |
| | GCGCTGMGTRTTCCCGCCTTKTGAC |
| | NNYARVYKRGCGARTCCKGGGAGRG |
| | GRYWGWTGACGTCAACAKTCVGGCT |
| | CCATGGCG (SEQ ID NO: 673) |
| Chiroptera | TGAGCTTCCCTCCGCCCTNBRVGDR |
| Alignment | RRDVVNNNBBBBDBNBGSVRRHTAT |
| consensus | ATRAGRNNCCYDYWYSKVWAGRCMT |
| sequence | TTYWHRRKTASGGTGAYTTCCCACA |
| 85% Identity | AYRCATAGCGACATGYAAATDHNNH |
| | NRGGYRTGCYTYCHCKGKCCYYNGY |
| | NRRMRNCDYCTYKNYNNNNMGNNNN |
| | NNSGNNCTGHGHRTTCCCGCCTTBT |
| | GRCNNYRRVYBRGCGARTNCDGGGA |
| | RRRGRYWGDTKAYGTCRNNNNNNNN |
| | NACWKTYVSGCTCSATGGCG |
| | (SEQ ID NO: 674) |
| Chiroptera | TGAGCTTCNCTCCGCCCTNBRVRDR |
| Alignment | RRDNNNNNNBBBDBNBVVVRRHTAT |
| consensus | ATRAGRNNCCYDBHYSKVDRGDYMT |
| sequence | TTHWHRRKKABGGTGAYTTCCCACA |
| 90%_Identity | AYRCAHAGCGACATGYAAATDHNNN |
| | NRGRYRTGYYTYCHCBGKCCYYNGY |
| | NRDMNNYDYNNNKNNNNNNMNNNNN |
| | NNSNNNSYGNBHDWTCCCGCCTTBN |
| | GRNNNYRNVBBRGCGARTNCDGGGA |
| | RVRRRYDGDTKAYGTVRNNNNNNNN |
| | NRYWBWBVSGCWYSATGGCG |
| | (SEQ ID NO: 675) |
| Chiroptera | TGAGCTTCNCTCCGCCCTNBRVRDR |
| Alignment | RDNNNNNNNNNNNBNNVVVVRNTAT |
| consensus | ATRAGRNNCCHDNNHBKVDDRDHMT |
| sequence | TTHNHRVDKABRGYRAYTTCCCAYA |
| 95%_Identity | AYRCMHRGCRAYATGYAAATDNNNN |
| | NRRDBDYGYYKBYNBNSNYYYBNNN |
| | NNNHNNNNNNNNNNNNNNNNNNNNN |
| | NNNNNNSNNNBHDNTCCCGCCTYNN |
| | NNNNNNNNVBNDRCRARTNCNRGGA |
| | RVRRRNDGNTKAYGYVRNNNNNNNN |
| | NRYWBHBNBGCDYNATGGCG |
| | (SEQ ID NO: 676) |
| Chiroptera | TGAGCTTCNCKCCGCCCYNNRVVNV |
| Alignment | VNNNNNNNNNNNNNNNVNNVVNTWW |
| consensus | AKVWRVNNNBYHNNNNBDNNNDNHM |
| sequence | YYTHNNVVNKABDGYRAYNTTCCCA |
| 99%_Identity | YRRBRCHHVGCRAYAYGYAAAWDNN |
| | NNNNDDBDYSYBNBYNNNNNBNNBN |
| | NNNNNNNNNNNNNNNNNNNNNNNNN |
| | NNNNNNNNNNNNNNNNNNNTYYYGB |
| | YHNNNNNNNNNNNNNNNNDRNDRVK |
| | NYNRGGRRVRVNNNNNNGNTBWYGH |
| | NNVNNNNNNNNNVYDNNNNNNNNYN |
| | ATGGCG (SEQ ID NO: 677) |
| Chiroptera | NVVNKABDGYRAYNTTCCCAYRRBR |
| Alignment | CHHVGCRAYAYGYAAAWDNNNNNND |
| consensus | DBDYSYBNBYNNNNNBNNBNNNNNN |
| sequence | NNNNNNNNNNNNNNNNNNNNNNNNN |
| 100%_Identity | NNNNNNNNNNNNNNTYYYGBYHNNN |
| | NNNNNNNNNNNNNDRNDRVKNYNRG |
| | GRRVRVNNNNNNGNTBWYGHNNVNN |
| | NNNNNNNVYDNNNNNNNNYNATGGC |
| | G(SEQ ID NO: 678) |

In some embodiments, the promoter comprises a nucleotide sequence having at least 85%, 90%, 95%, 96%, 97%, 98%, 99%, or 100% identity to nucleotides 20-253 of any one of SEQ ID NOs: 673-678 or a functional fragment or variant (e.g., codon optimized) thereof.

### Dermoptera H1 Promoters

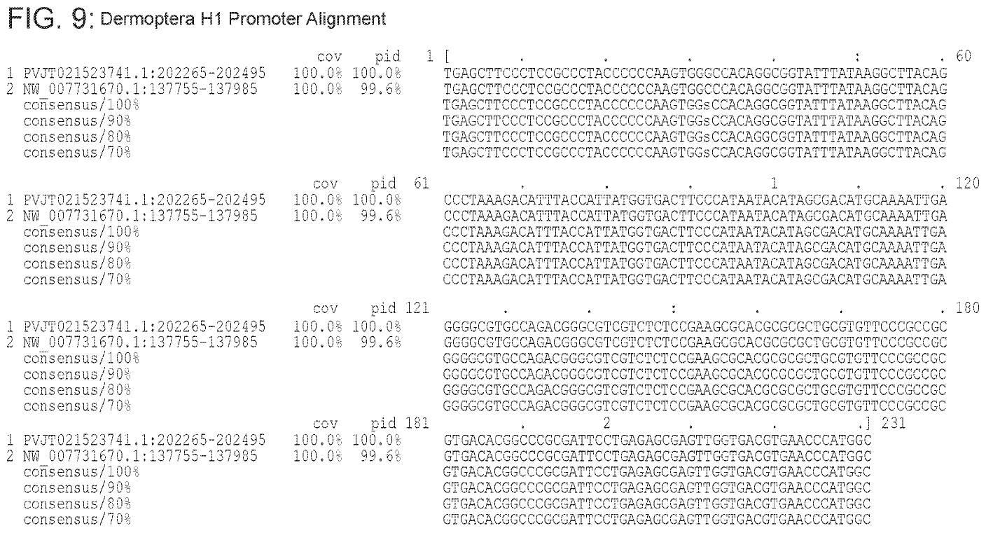

In certain embodiments, the promoter comprises a Dermoptera H1 promoter. An alignment of Dermoptera H1 promoter sequences is provided in FIG. 9 (wherein sequences numbered 1-2 in FIG. 9 correspond to SEQ ID NOs: 679 and 680, and consensus/100%, consensus/90%, consensus/80% and consensus/70% correspond to SEQ ID NOs: 1827-1830, respectively). In some embodiments, the promoter comprises a nucleotide sequence having at least 85%, 90%, 95%, 96%, 97%, 98%, 99%, or 100% identity to nucleotides 20-227 of any one of the sequences in FIG. 9 or a functional fragment or variant (e.g., codon optimized) thereof.

In certain embodiments, the Dermoptera H1 promoter comprises

TGAGCTTCCCTCCGCCCTACCCCCCAAGTGGSCCACAGG
CGGTATTTATAAGGCTTACAGCCCTAAAGACATTTACCA
TTATGGTGACTTCCCATAATACATAGCGACATGCAAAAT
TGAGGGGCGTGCCAGACGGGCGTCGTCTCTCCGAAGCGC
ACGCGCGCTGCGTGTTCCCGCCGCGTGACACGGCCCGCG
ATTCCTGAGAGCGAGTTGGTGACGTGAACCCATGGC
(SEQ ID NO: 681; Dermoptera Alignment
consensus sequence 100%_Identity)

In some embodiments, the promoter comprises a nucleotide sequence having at least 85%, 90%, 95%, 96%, 97%, 98%, 99%, or 100% identity to nucleotides 20-227 of SEQ ID NO: 681 or a functional fragment or variant (e.g., codon optimized) thereof.

### Hyracoidae H1 Promoters

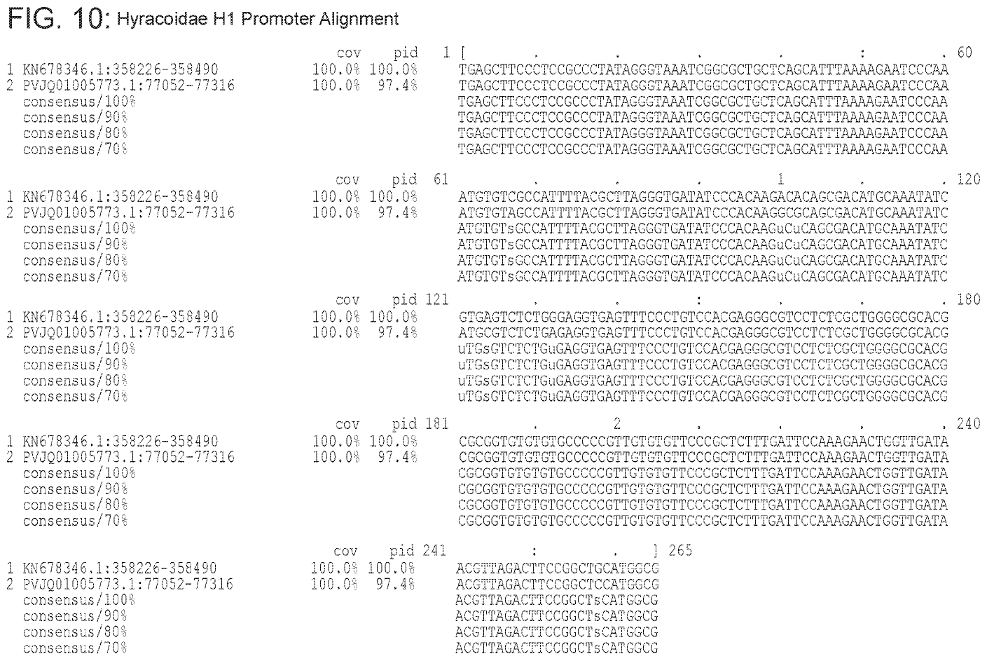

In certain embodiments, the promoter comprises an Hyracoidae H1 promoter. An alignment of Hyracoidae H1 promoter sequences is provided in FIG. 10 (wherein sequences numbered 1-2 in FIG. 10 correspond to SEQ ID NOs: 682 and 683, and consensus/100%, consensus/90%, consensus/80% and consensus/70% correspond to SEQ ID NOs: 1831-1834, respectively). In some embodiments, the promoter comprises a nucleotide sequence having at least 85%, 90%, 95%, 96%, 97%, 98%, 99%, or 100% identity to nucleotides 20-259 of any one of the sequences in FIG. 10 or a functional fragment or variant (e.g., codon optimized) thereof.

### Insectavora H1 Promoters

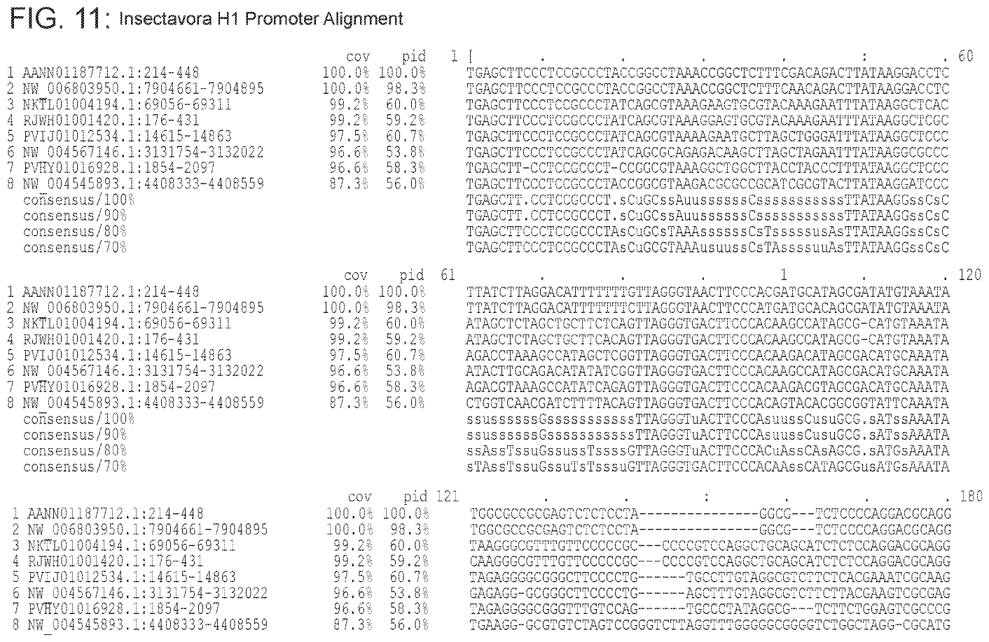

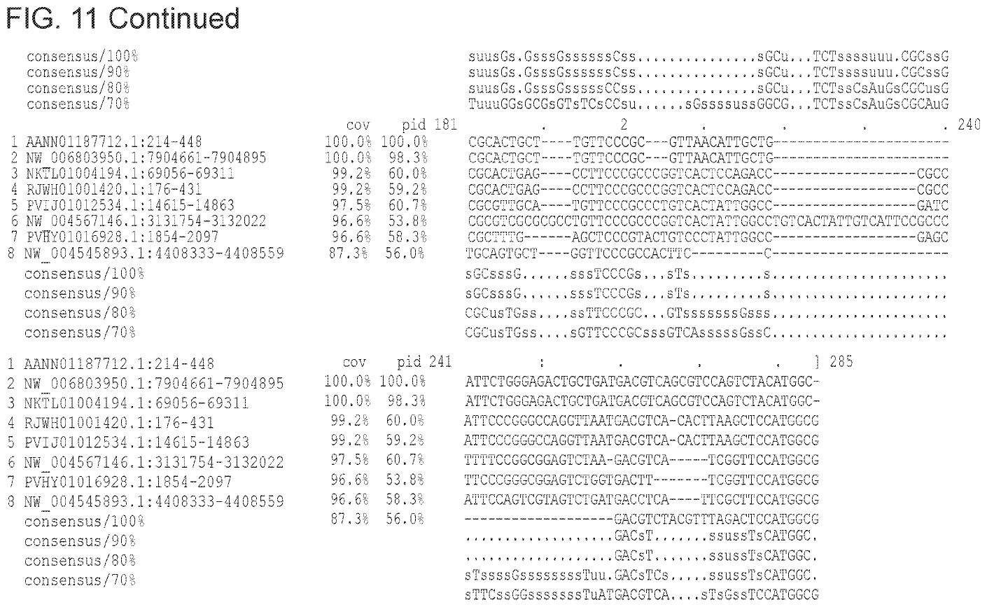

In certain embodiments, the promoter comprises an Insectavora H1 promoter. An alignment of Insectavora H1 promoter sequences is provided in FIG. 11 (wherein sequences numbered 1-8 in FIG. 11 correspond to SEQ ID NOs: 684-691, and consensus/100%, consensus/90%, consensus/80% and consensus/70% correspond to SEQ ID NOs: 1835-1838, respectively). In some embodiments, the promoter comprises a nucleotide sequence having at least 85%, 90%, 95%, 96%, 97%, 98%, 99%, or 100% identity to nucleotides 20-279 of any one of the sequences in FIG. 11 or a functional fragment or variant (e.g., codon optimized) thereof.

In certain embodiments, the Insectavora H1 promoter comprises a sequence selected from those in TABLE 9.

TABLE 9

| | |
|---|---|
| Insectavora | TGAGCTTCCCTCCGCCCTAYCRGCG |
| Alignment | TAAAVSRRBKCKTASMWMRRAYTTA |
| consensus | TAAGGMYCYCWTASYTHWRGMYRTW |
| sequence | TYWYDGTTAGGGTGACTTCCCACAA |
| 75%_Identity | KMCATAGCGAYATGYAAATATRRVG |
| | GSGCGKGTYTCYCCKVGGTCYYHGY |
| | YYWGKMGGCGKCWTCTYHCSARGWC |
| | GCARGCGCRYTGMKCGCCYGTTCCC |
| | GCCCKGTCAMYMYWGVYCTGTCACT |
| | ATTGTCATTCCSRBCWTTCYSGGVS |
| | VMKKYTRATGACGTCARCRYYTMGK |
| | YTCCATGGCG |
| | (SEQ ID NO: 692) |
| Insectavora | TGAGCTTCCCTCCGCCCTAYCRGCS |
| Alignment | TAAAVVVNBKCKTWSMWMRNAYTTA |
| consensus | TAAGGMYCNCWKABYTHWRGMYRYW |
| sequence | TYWYDGTTAGGGTRACTTCCCACRA |
| 85%_Identity | KVCAYAGCGRYATGYAAATABRRVG |
| | SSGYKDGYYYVYCCNVGGTCYYHGB |
| | YYWRKVKGCRKSDTCTYHCSARGWC |
| | GCVNGCGCRYTGMKCGCCNSTTCCC |
| | GCMMBGTYAMYMYWGVYSTGTCACT |
| | ATTGTCATTCCSVBCWTTCYSGGVS |
| | VMKKYTRATGACBTCARCRYYYMRN |
| | YTMCATGGCG |
| | (SEQ ID NO: 693) |
| Insectavora | TGAGCTTCCCTCCGCCCTAYCRGCS |
| Alignment | YARRVVVNNBCKYWBVDVVNMYTTA |
| consensus | TAAGGMBCNCHKRBBYNHVGMYVYW |
| sequence | KHWBDSTTAGGGTRACTTCCCAYRR |
| 90%_Identity | KVCRYRGCGRYATKYAAATABRRVG |
| | SSGYKDGYYYVBYCNVGGTCYYHGB |
| | YYWRKVKGCRKSDTCTBNYBRRRWC |
| | GCVNGYGCDBYGMDCGCCNSYTCCC |
| | GYMMBKTYMMYMYWGVYSTGTCACT |
| | ATTGTCATTCCSVBCWTYYYVGKVS |
| | NMKKYTRRTGACBTCWRCRYYYMRN |
| | YTMCATGGCG |
| | (SEQ ID NO: 694) |
| Insectavora | TGAGCTTCCCTCCGCCCTAYCRGCS |
| Alignment | YARRVVVNNBCKYWBVDVVNMYTTA |
| consensus | TAAGGMBCNCHKRBBYNHVGMYVYW |
| sequence | KHWBDSTTAGGGTRACTTCCCAYRR |
| 95% Identity | KVCRYRGCGRYATKYAAATABRRVG |
| | SSGYKDGYYYVBYCNVGGTCYYHGB |
| | YYWRKVKGCRKSDTCTBNYBRRRWC |
| | GCVNGYGCDBYGMDCGCCNSYTCCC |
| | GYMMBKTYMMYMYWGVYSTGTCACT |
| | ATTGTCATTCCSVBCWTYYYVGKVS |
| | NMKKYTRRTGACBTCWRCRYYYMRN |
| | YTMCATGGCG |
| | (SEQ ID NO: 695) |
| Insectavora | TGAGCTTCCCTCCGCCCTAYCRGCS |
| Alignment | YARRVVVNNBCKYWBVDVVNMYTTA |
| consensus | TAAGGMBCNCHKRBBYNHVGMYVYW |
| sequence | KHWBDSTTAGGGTRACTTCCCAYRR |
| 99%_Identity | KVCRYRGCGRYATKYAAATABRRVG |
| | SSGYKDGYYYVBYCNVGGTCYYHGB |
| | YYWRKVKGCRKSDTCTBNYBRRRWC |
| | GCVNGYGCDBYGMDCGCCNSYTCCC |
| | GYMMBKTYMMYMYWGVYSTGTCACT |
| | ATTGTCATTCCSVBCWTYYYVGKVS |
| | NMKKYTRRTGACBTCWRCRYYYMRN |
| | YTMCATGGCG |
| | (SEQ ID NO: 696) |
| Insectavora | TGAGCTTCCCTCCGCCCTAYCRGCS |
| Alignment | YARRVVVNNBCKYWBVDVVNMYTTA |
| consensus | TAAGGMBCNCHKRBBYNHVGMYVYW |
| sequence | KHWBDSTTAGGGTRACTTCCCAYRR |
| 100%_Identity | KVCRYRGCGRYATKYAAATABRRVG |
| | SSGYKDGYYYVBYCNVGGTCYYHGB |
| | YYWRKVKGCRKSDTCTBNYBRRRWC |
| | GCVNGYGCDBYGMDCGCCNSYTCCC |
| | GYMMBKTYMMYMYWGVYSTGTCACT |
| | ATTGTCATTCCSVBCWTYYYVGKVS |
| | NMKKYTRRTGACBTCWRCRYYYMRN |
| | YTMCATGGCG |
| | (SEQ ID NO: 697) |

In some embodiments, the promoter comprises a nucleotide sequence having at least 85%, 90%, 95%, 96%, 97%, 98%, 99%, or 100% identity to nucleotides 20-278 of any one of SEQ ID NOs: 692-697 or a functional fragment or variant (e.g., codon optimized) thereof.

### Lagomorpha H1 Promoters

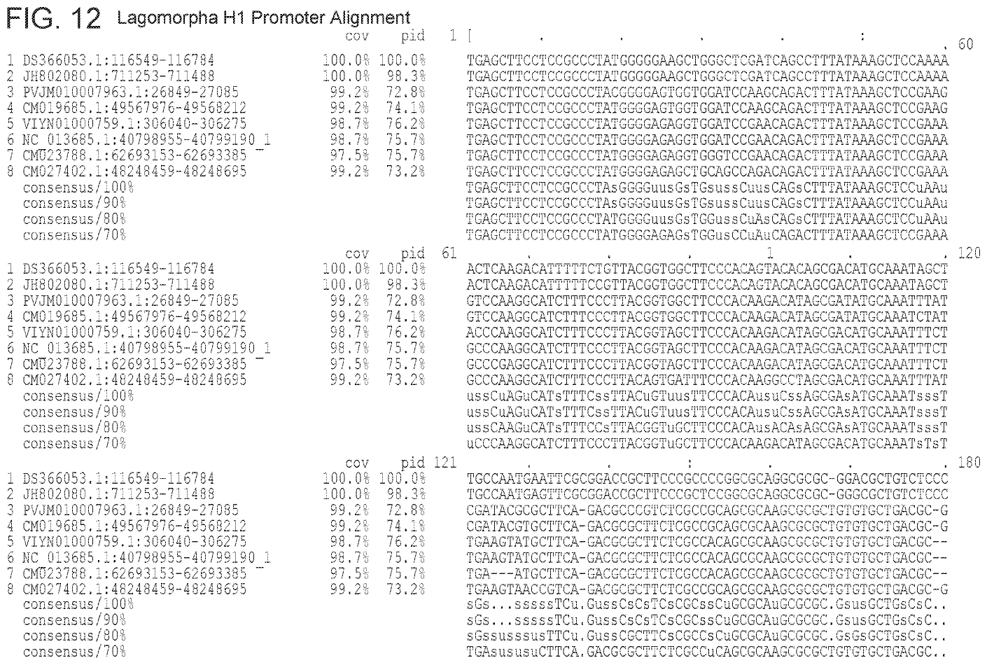

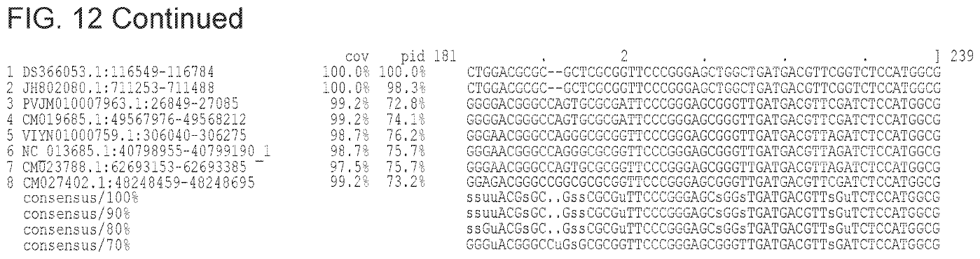

In certain embodiments, the promoter comprises a Lagomorpha H1 promoter. An alignment of Lagomorpha H1 promoter sequences is provided in FIG. 12 (wherein sequences numbered 1-8 in FIG. 12 correspond to SEQ ID NOs: 698-705, and consensus/100%, consensus/90%, consensus/80% and consensus/70% correspond to SEQ ID NOs: 1839-1842, respectively). In some embodiments, the promoter comprises a nucleotide sequence having at least 85%, 90%, 95%, 96%, 97%, 98%, 99%, or 100% identity to nucleotides 20-233 of any one of the sequences in FIG. 12 or a functional fragment or variant (e.g., codon optimized) thereof.

In certain embodiments, the Lagomorpha H1 promoter comprises a sequence selected from those in TABLE 10.

TABLE 10

| | |
|---|---|
| Lagomorpha | TGAGCTTCCTCCGCCCTATGGGGAG |
| Alignment | AGSTGGRYCCRADCAGACTTTATAA |
| consensus | AGCTCCGAAARCCCAAGGCATCTTT |
| sequence | CCCTTACGGTRGCTTCCCACAAGAC |
| 75%_Identity | ATAGCGACATGCAAATWTMTTGAHR |
| | HDKRCTTCACGACGCGCTTCTCGCC |
| | RCAGCGCAAGCGCGCTGTGTGCTGA |
| | CGCCSGGGRACGGGCCAGYGCGCGG |
| | TTCCCGGGAGCGGGTTGATGACGTT |
| | MGATCTCCATGGCG |
| | (SEQ ID NO: 706) |
| Lagomorpha | TGAGCTTCCTCCGCCCTATGGGGRR |
| Alignment | WGSTGGRYYCRADCAGMCTTTATAA |
| consensus | AGCTCCRAARRYYCAAGRCATYTTT |
| sequence | CCSTTACGGTRGCTTCCCACARKAC |
| 85% Identity | AYAGCGAYATGCAAATWKMTYGMHR |
| | HDNRVTTCRCGRMSCGCTTCYCGCC |
| | VCRGCGCARGCGCGCTGKGYGCTGW |
| | CKCCSSKGRACGSGCCRGBKCGCGR |
| | TTCCCGGGAGCKGGYTGATGACGTT |
| | MGRTCTCCATGGCG |
| | (SEQ ID NO: 707) |
| Lagomorpha | TGAGCTTCCTCCGCCCTAYGGGGRR |
| Alignment | WGSTGSRBYCRRDCAGMCTTTATAA |
| consensus | AGCTCCRAARRYYCRAGRCATYTTT |
| sequence | CYSTTACRGTRRYTTCCCACARKRC |
| 90% Identity | MYAGCGAYATGCAAATHKMTYGMHR |
| | HDNVVKTCRCGRMSCSCKTCYCGCY |
| | VCRGCGCARGCGCGCTGKRYGCTGW |
| | CKCCSSKRRACGSGCCRGBKCGCGR |
| | TTCCCGGGAGCKGGYTGATGACGTT |
| | MGRTCTCCATGGCG |
| | (SEQ ID NO: 708) |
| Lagomorpha | TGAGCTTCCTCCGCCCTAYGGGGRR |
| Alignment | WGSTGSRBYCRRDCAGMCTTTATAA |
| consensus | AGCTCCRAARRYYCRAGRCATYTTT |
| sequence | CYSTTACRGTRRYTTCCCACARKRC |
| 95%_Identity | MYAGCGAYATGCAAATHKMTYGMHR |
| | HDNVVKTCRCGRMSCSCKTCYCGCY |
| | VCRGCGCARGCGCGCTGKRYGCTGW |
| | CKCCSSKRRACGSGCCRGBKCGCGR |
| | TTCCCGGGAGCKGGYTGATGACGTT |
| | MGRTCTCCATGGCG |
| | (SEQ ID NO: 709) |
| Lagomorpha | TGAGCTTCCTCCGCCCTAYGGGGRR |
| Alignment | WGSTGSRBYCRRDCAGMCTTTATAA |
| consensus | AGCTCCRAARRYYCRAGRCATYTTT |
| sequence | CYSTTACRGTRRYTTCCCACARKRC |
| 99%_Identity | MYAGCGAYATGCAAATHKMTYGMHR |
| | HDNVVKTCRCGRMSCSCKTCYCGCY |
| | VCRGCGCARGCGCGCTGKRYGCTGW |
| | CKCCSSKRRACGSGCCRGBKCGCGR |
| | TTCCCGGGAGCKGGYTGATGACGTT |
| | MGRTCTCCATGGCG |
| | (SEQ ID NO: 710) |
| Lagomorpha | TGAGCTTCCTCCGCCCTAYGGGGRR |
| Alignment | WGSTGSRBYCRRDCAGMCTTTATAA |
| consensus | AGCTCCRAARRYYCRAGRCATYTTT |
| sequence | CYSTTACRGTRRYTTCCCACARKRC |
| 100%_Identity | MYAGCGAYATGCAAATHKMTYGMHR |
| | HDNVVKTCRCGRMSCSCKTCYCGCY |
| | VCRGCGCARGCGCGCTGKRYGCTGW |
| | CKCCSSKRRACGSGCCRGBKCGCGR |
| | TTCCCGGGAGCKGGYTGATGACGTT |
| | MGRTCTCCATGGCG |
| | (SEQ ID NO: 711) |

In some embodiments, the promoter comprises a nucleotide sequence having at least 85%, 90%, 95%, 96%, 97%, 98%, 99%, or 100% identity to nucleotides 20-233 of any one of SEQ ID NOs: 706-711 or a functional fragment or variant (e.g., codon optimized) thereof.

### Marsupial H1 Promoters

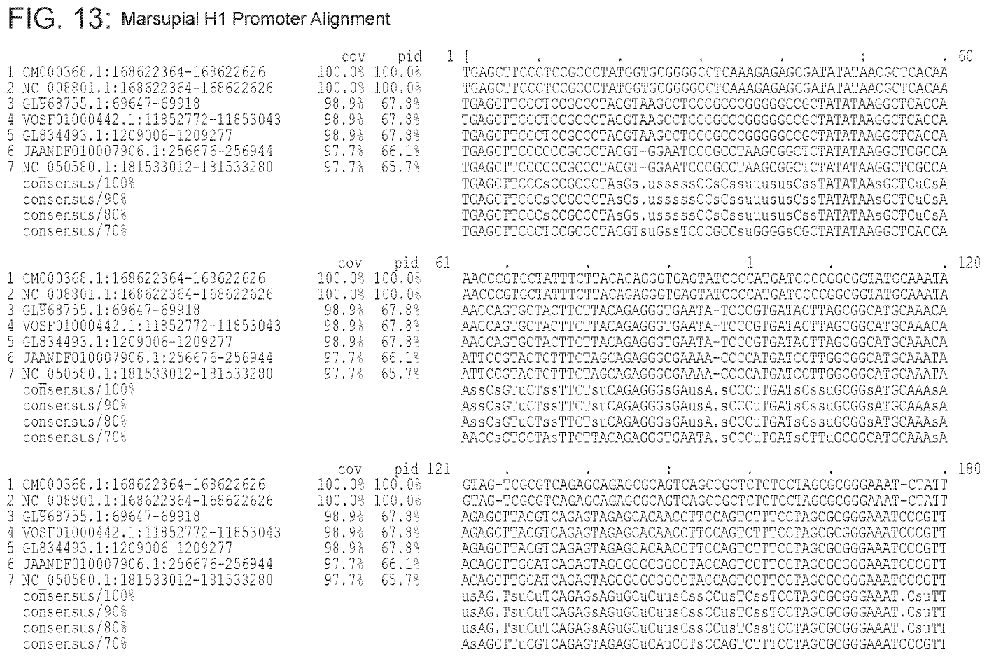

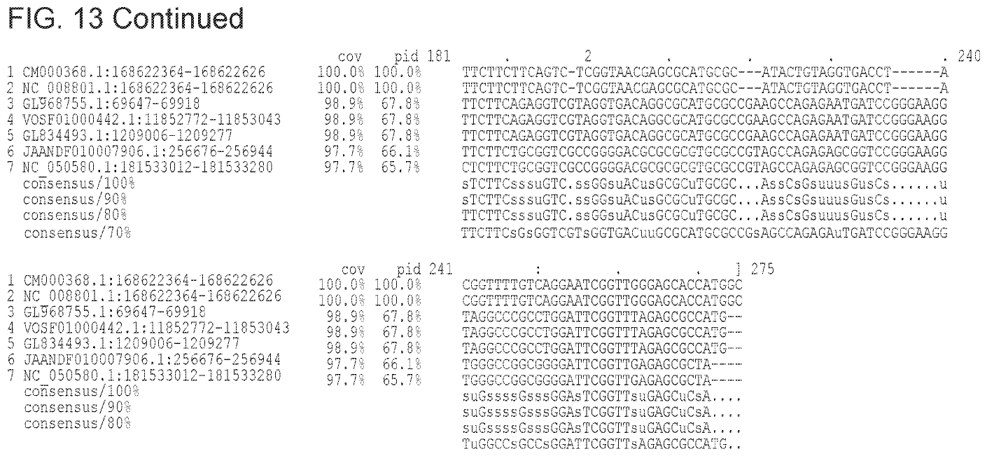

In certain embodiments, the promoter comprises a Marsupial H1 promoter. An alignment of Marsupial H1 promoter sequences is provided in FIG. 13 (wherein sequences numbered 1-7 in FIG. 13 correspond to SEQ ID NOs: 712-718, and consensus/100%, consensus/90%, consensus/80% and consensus/70% correspond to SEQ ID NOs: 1843-1846, respectively). In some embodiments, the promoter comprises a nucleotide sequence having at least 85%, 90%, 95%, 96%, 97%, 98%, 99%, or 100% identity to nucleotides 20-270 of any one of the sequences in FIG. 13 or a functional fragment or variant (e.g., codon optimized) thereof.

In certain embodiments, the Marsupial H1 promoter comprises a sequence selected from those in TABLE 11.

TABLE 11

| | |
|---|---|
| Marsupial | TGAGCTTCCCYCCGCCCTAYGKNRS |
| Alignment | VVKSCCKCMHRRRSRSCKMTATATA |
| consensus | ASGCTCRCMAAWYCMGTRCTMYTTC |
| sequence | TWRCAGAGGGYGARWANYCCCRTGA |
| 75%_Identity | TMCYYRGCGGYATGCAAAYARBAGN |
| | TYRCRTCAGAGYAGRGCRCRRYCWD |
| | CCRSTCYYTCCTAGCGCGGGAAATN |
| | CYRTTTTCTTCWKMRGTCNYMGGKR |
| | ACRVGCGCRTGCGCNNNAKMCWGWR |
| | RRYGRYCYNNNNNNRYRGKYYBGYS |
| | DGGAWTCGGTTKRGAGCRCYATGGC |
| | (SEQ ID NO: 719) |
| Marsupial | TGAGCTTCCCYCCGCCCTAYGKNRS |
| Alignment | VVKSCCKCMHRRRSRSCKMTATATA |
| consensus | ASGCTCRCMAAWYCMGTRCTMYTTC |
| sequence | TWRCAGAGGGYGARWANYCCCRTGA |
| | TMCYYRGCGGYATGCAAAYARBAGN |
| | TYRCRTCAGAGYAGRGCRCRRYCWD |
| | CCRSTCYYTCCTAGCGCGGGAAATN |
| | CYRTTTTCTTCWKMRGTCNYMGGKR |
| | ACRVGCGCRTGCGCNNNAKMCWGWR |
| | RRYGRYCYNNNNNNRYRGKYYBGYS |
| | DGGAWTCGGTTKRGAGCRCYATGGC |
| | (SEQ ID NO: 720) |
| 85%_Identity | |
| Marsupial | TGAGCTTCCCYCCGCCCTAYGKNRS |
| Alignment | VVKSCCKCMHRRRSRSCKMTATATA |
| consensus | ASGCTCRCMAAWYCMGTRCTMYTTC |
| sequence | TWRCAGAGGGYGARWANYCCCRTGA |
| 90% Identity | TMCYYRGCGGYATGCAAAYARBAGN |
| | TYRCRTCAGAGYAGRGCRCRRYCWD |
| | CCRSTCYYTCCTAGCGCGGGAAATN |
| | CYRTTYTCTTCWKMRGTCNYMGGKR |
| | ACRVGCGCRTGCGCNNNAKMCWGWR |
| | RRYGRYCYNNNNNNRYRGKYYBGYS |
| | DGGAWTCGGTTKRGAGCRCYATGGC |
| | (SEQ ID NO: 721) |
| Marsupial | TGAGCTTCCCYCCGCCCTAYGKNRS |
| Alignment | VVKSCCKCMHRRRSRSCKMTATATA |
| consensus | ASGCTCRCMAAWYCMGTRCTMYTTC |
| sequence | TWRCAGAGGGYGARWANYCCCRTGA |
| 95%_Identity | TMCYYRGCGGYATGCAAAYARBAGN |
| | TYRCRTCAGAGYAGRGCRCRRYCWD |
| | CCRSTCYYTCCTAGCGCGGGAAATN |
| | CYRTTYTCTTCWKMRGTCNYMGGKR |
| | ACRVGCGCRTGCGCNNNAKMCWGWR |
| | RRYGRYCYNNNNNNRYRGKYYBGYS |
| | DGGAWTCGGTTKRGAGCRCYATGGC |
| | (SEQ ID NO: 722) |
| Marsupial | TGAGCTTCCCYCCGCCCTAYGKNRS |
| Alignment | VVKSCCKCMHRRRSRSCKMTATATA |
| consensus | ASGCTCRCMAAWYCMGTRCTMYTTC |
| sequence | TWRCAGAGGGYGARWANYCCCRTGA |
| 99%_Identity | TMCYYRGCGGYATGCAAAYARBAGN |
| | TYRCRTCAGAGYAGRGCRCRRYCWD |
| | CCRSTCYYTCCTAGCGCGGGAAATN |
| | CYRTTYTCTTCWKMRGTCNYMGGKR |
| | ACRVGCGCRTGCGCNNNAKMCWGWR |
| | RRYGRYCYNNNNNNRYRGKYYBGYS |
| | DGGAWTCGGTTKRGAGCRCYATGGC |
| | (SEQ ID NO: 723) |
| Marsupial | TGAGCTTCCCYCCGCCCTAYGKNRS |
| Alignment | VVKSCCKCMHRRRSRSCKMTATATA |
| consensus | ASGCTCRCMAAWYCMGTRCTMYTTC |
| sequence | TWRCAGAGGGYGARWANYCCCRTGA |
| 100%_Identity | TMCYYRGCGGYATGCAAAYARBAGN |
| | TYRCRTCAGAGYAGRGCRCRRYCWD |
| | CCRSTCYYTCCTAGCGCGGGAAATN |
| | CYRTTYTCTTCWKMRGTCNYMGGKR |
| | ACRVGCGCRTGCGCNNNAKMCWGWR |
| | RRYGRYCYNNNNNNRYRGKYYBGYS |
| | DGGAWTCGGTTKRGAGCRCYATGGC |
| | (SEQ ID NO: 724) |

In some embodiments, the promoter comprises a nucleotide sequence having at least 85%, 90%, 95%, 96%, 97%, 98%, 99%, or 100% identity to nucleotides 20-270 of any one of SEQ ID NOs: 719-724 or a functional fragment or variant (e.g., codon optimized) thereof.

### Pangolin H1 Promoters

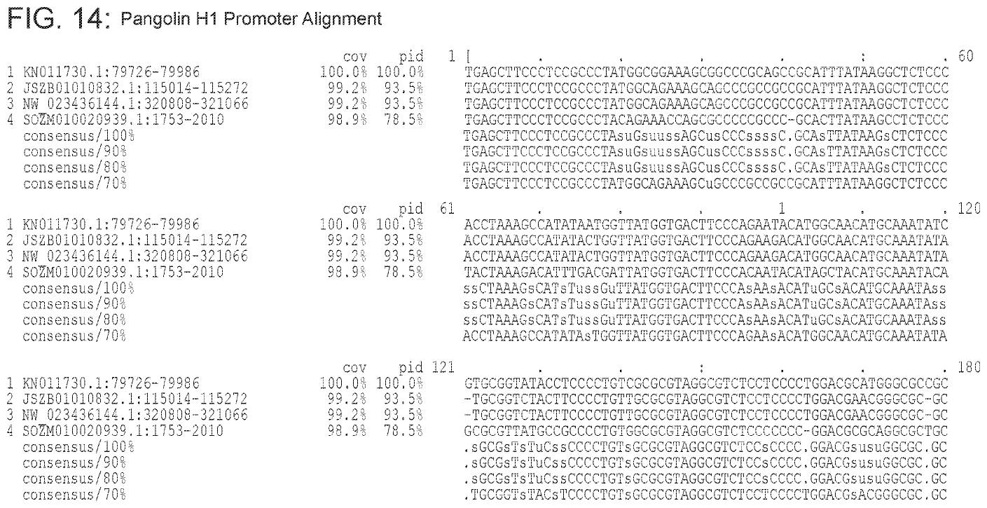

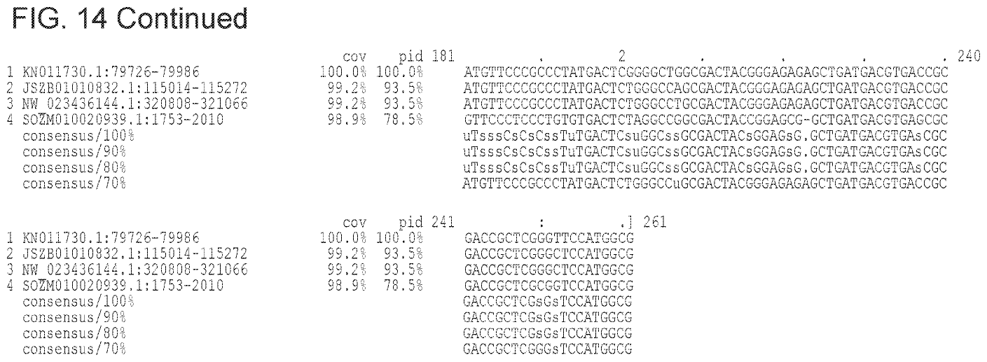

In certain embodiments, the promoter comprises an Pangolin H1 promoter. An alignment of Pangolin H1 promoter sequences is provided in FIG. 14 (wherein sequences numbered 1-4 in FIG. 14 correspond to SEQ ID NOs: 725-728, and consensus/100%, consensus/90%, consensus/80% and consensus/70% correspond to SEQ ID NOs: 1847-1850, respectively). In some embodiments, the promoter comprises a nucleotide sequence having at least 85%, 90%, 95%, 96%, 97%, 98%, 99%, or 100% identity to nucleotides 20-255 of any one of the sequences in FIG. 14 or a functional fragment or variant (e.g., codon optimized) thereof.

In certain embodiments, the Pangolin H1 promoter comprises a sequence selected from those in TABLE 12.

TABLE 12

| | |
|---|---|
| Pangolin | TGAGCTTCCCTCCGCCCTATGGCAG |
| Alignment | AAAGCRGCCCGCCGCCGCATTTATA |
| consensus | AGGCTCTCCCACCTAAAGCCATATA |
| sequence | MTGGTTATGGTGACTTCCCAGAAKA |
| 75% Identity | CATGGCAACATGCAAATATANTGCG |
| | GTMTACYTCCCCTGTBGCGCGTAGG |
| | CGTCTCCTCCCCTGGACGMACGGGC |
| | GCNGCATGTTCCCGCCCTATGACTC |
| | TGGGCCDGCGACTACGGGAGAGAGC |
| | TGATGACGTGACCGCGACCGCTCGG |
| | GBTCCATGGCG |
| | (SEQ ID NO: 729) |
| Pangolin | TGAGCTTCCCTCCGCCCTAYRGMRR |
| Alignment | MMAGCRSCCCSSMSCNGCAYTTATA |
| consensus | AGSCTCTCCCWMCTAAAGMCATWTR |
| sequence | MYGRTTATGGTGACTTCCCASAAKA |
| 85%_Identity | CATRGCWACATGCAAATAYMNYGCG |
| | KTMTRCYKCCCCTGTBGCGCGTAGG |
| | CGTCTCCYCCCCNGGACGMRYRGGC |
| | GCNGCRTKYYCYCSCYSTRTGACTC |
| | KRGGCYDGCGACTACSGGAGMGNGC |
| | TGATGACGTGASCGCGACCGCTCGS |
| | GBTCCATGGCG |
| | (SEQ ID NO: 730) |
| Pangolin | TGAGCTTCCCTCCGCCCTAYRGMRR |
| Alignment | MMAGCRSCCCSSMSCNGCAYTTATA |
| consensus | AGSCTCTCCCWMCTAAAGMCATWTR |
| sequence | MYGRTTATGGTGACTTCCCASAAKA |
| 90%_Identity | CATRGCWACATGCAAATAYMNYGCG |
| | KTMTRCYKCCCCTGTBGCGCGTAGG |
| | CGTCTCCYCCCCNGGACGMRYRGGC |
| | GCNGCRTKYYCYCSCYSTRTGACTC |
| | KRGGCYDGCGACTACSGGAGMGNGC |
| | TGATGACGTGASCGCGACCGCTCGS |
| | GBTCCATGGCG |
| | (SEQ ID NO: 731) |
| Pangolin | TGAGCTTCCCTCCGCCCTAYRGMRR |
| Alignment | MMAGCRSCCCSSMSCNGCAYTTATA |
| consensus | AGSCTCTCCCWMCTAAAGMCATWTR |
| sequence | MYGRTTATGGTGACTTCCCASAAKA |
| 95%_Identity | CATRGCWACATGCAAATAYMNYGCG |
| | KTMTRCYKCCCCTGTBGCGCGTAGG |
| | CGTCTCCYCCCCNGGACGMRYRGGC |
| | GCNGCRTKYYCYCSCYSTRTGACTC |
| | KRGGCYDGCGACTACSGGAGMGNGC |
| | TGATGACGTGASCGCGACCGCTCGS |
| | GBTCCATGGCG |
| | (SEQ ID NO: 732) |
| Pangolin | TGAGCTTCCCTCCGCCCTAYRGMRR |
| Alignment | MMAGCRSCCCSSMSCNGCAYTTATA |
| consensus | AGSCTCTCCCWMCTAAAGMCATWTR |
| sequence | MYGRTTATGGTGACTTCCCASAAKA |
| 99%_Identity | CATRGCWACATGCAAATAYMNYGCG |
| | KTMTRCYKCCCCTGTBGCGCGTAGG |
| | CGTCTCCYCCCCNGGACGMRYRGGC |
| | GCNGCRTKYYCYCSCYSTRTGACTC |
| | KRGGCYDGCGACTACSGGAGMGNGC |
| | TGATGACGTGASCGCGACCGCTCGS |
| | GBTCCATGGCG |
| | (SEQ ID NO: 733) |
| Pangolin | TGAGCTTCCCTCCGCCCTAYRGMRR |
| Alignment | MMAGCRSCCCSSMSCNGCAYTTATA |
| consensus | AGSCTCTCCCWMCTAAAGMCATWTR |
| sequence | MYGRTTATGGTGACTTCCCASAAKA |
| 100% Identity | CATRGCWACATGCAAATAYMNYGCG |
| | KTMTRCYKCCCCTGTBGCGCGTAGG |
| | CGTCTCCYCCCCNGGACGMRYRGGC |
| | GCNGCRTKYYCYCSCYSTRTGACTC |
| | KRGGCYDGCGACTACSGGAGMGNGC |
| | TGATGACGTGASCGCGACCGCTCGS |
| | GBTCCATGGCG |
| | (SEQ ID NO: 734) |

In some embodiments, the promoter comprises a nucleotide sequence having at least 85%, 90%, 95%, 96%, 97%, 98%, 99%, or 100% identity to nucleotides 20-255 of any one of SEQ ID NOs: 729-734 or a functional fragment or variant (e.g., codon optimized) thereof.

### Perissodactyla H1 Promoters

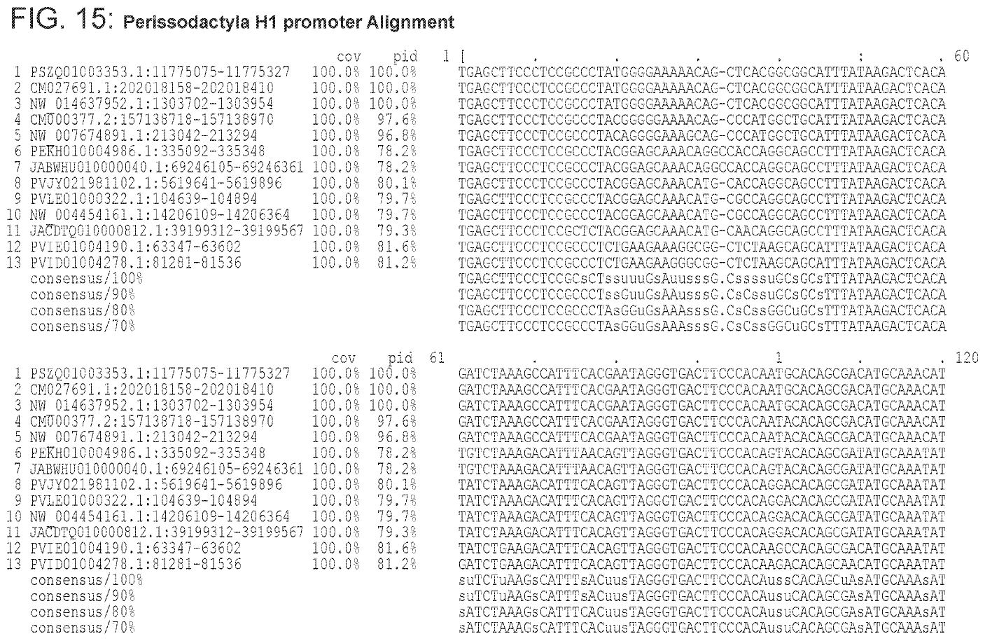

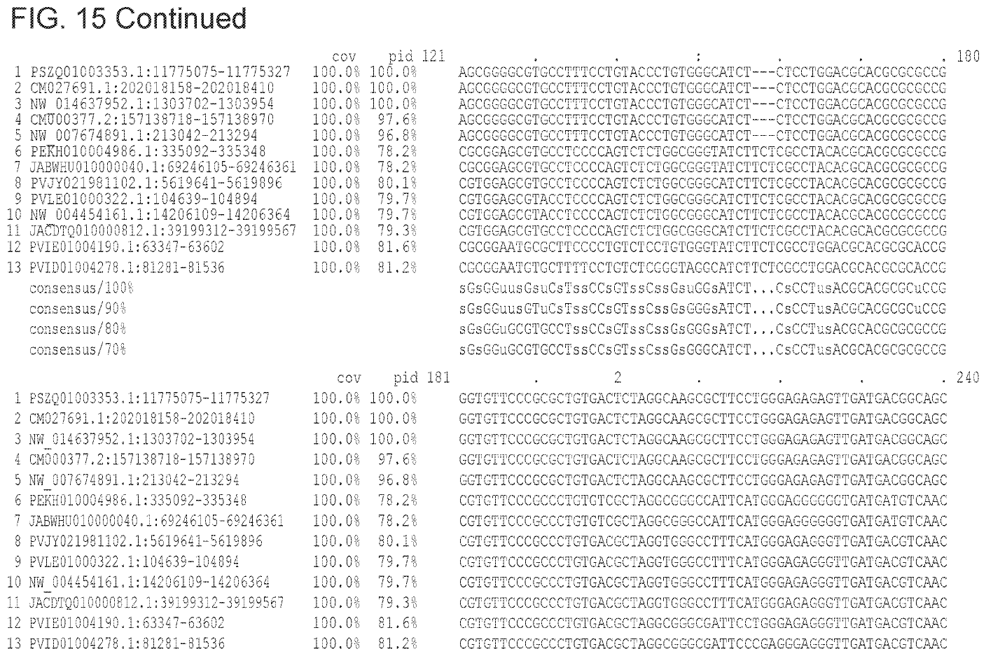

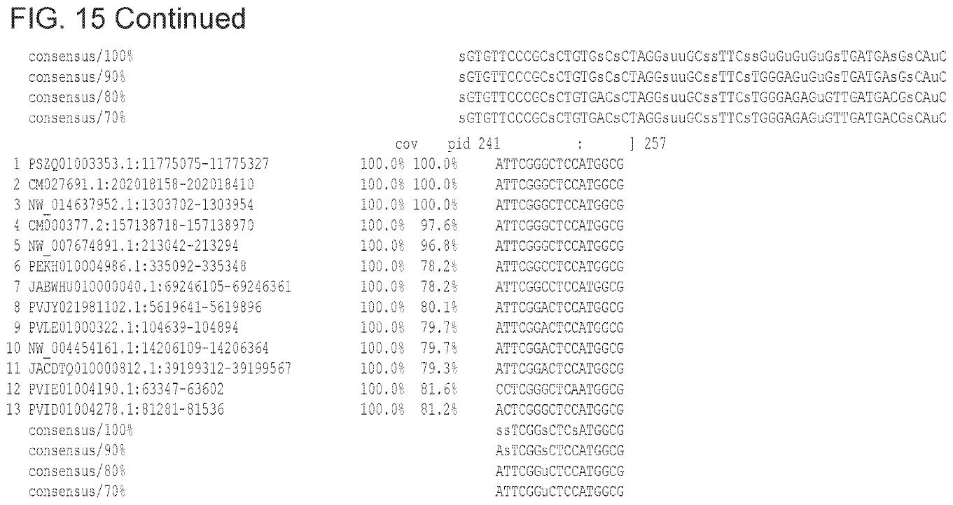

In certain embodiments, the promoter comprises an Perissodactyla H1 promoter. An alignment of Perissodactyla H1 promoter sequences is provided in FIG. 15 (wherein sequences numbered 1-13 in FIG. 15 correspond to SEQ ID NOs: 735-747, and consensus/100%, consensus/90%, consensus/80% and consensus/70% correspond to SEQ ID NOs: 1851-1854, respectively). In some embodiments, the promoter comprises a nucleotide sequence having at least 85%, 90%, 95%, 96%, 97%, 98%, 99%, or 100% identity to nucleotides 20-251 of any one of the sequences in FIG. 15 or a functional fragment or variant (e.g., codon optimized) thereof.

In certain embodiments, the Perissodactyla H1 promoter comprises a sequence selected from those in TABLE 13.

TABLE 13

| | |
|---|---|
| Perissodactyla | TGAGCTTCCCTCCGCCCTAYGGRGM |
| Alignment | AAAMMDGCNCMMGGCRGCMTTTATA |
| consensus | AGACTCACAKATCTAAAGMCATTTC |
| sequence | ACRRWTAGGGTGACTTCCCACARKR |
| 75% Identity | CACAGCGAYATGCAAAYATMGYGGR |
| | GCGTGCCTYYCCWGTMYCYKGYGGG |
| | CATCTNNNCKCCTRSACGCACGCGC |
| | GCCGSGTGTTCCCGCSCTGTGACKC |
| | TAGGYRRGCSHTTCMTGGGAGAGRG |
| | TTGATGACGKCARCATTCGGRCTCC |
| | ATGGCG |
| | (SEQ ID NO: 748) |
| Perissodactyla | TGAGCTTCCCTCCGCCCTAYGGRGM |
| Alignment | AAAVMDGCNCMMGGCRGCMTTTATA |
| consensus | AGACTCACAKATCTAAAGMCATTTC |
| sequence | ACRRWTAGGGTGACTTCCCACARKR |
| 85%_Identity | CACAGCGAYATGCAAAYATMGYGGR |
| | GCGTGCCTYYCCWGTMYCYKGYGGG |
| | YATCTNNNCKCCTRSACGCACGCGC |
| | GCCGSGTGTTCCCGCSCTGTGACKC |
| | TAGGYRRGCSHTTCMTGGGAGAGRG |
| | TTGATGACGKCARCATTCGGRCTCC |
| | ATGGCG |
| | (SEQ ID NO: 749) |
| Perissodactyla | TGAGCTTCCCTCCGCCCTMYGRRGV |
| Alignment | AARVMDGNCNCHHRGCDGCMTTTAT |
| consensus | AAGACTCACAKRTCTRAAGMCATTT |
| sequence | MACRRWTAGGGTGACTTCCCACARK |
| 90%_Identity | RCACAGCGAYATGCAAAYATMGYGG |
| | RRYGTRCYTYYCCWGTMYCYKGYGG |
| | GYATCTNNNCKCCTRSACGCACGCG |
| | CRCCGSGTGTTCCCGCSCTGTGWCK |
| | CTAGGYRRGCSHTTCMTGGGAGRGR |
| | GKTGATGAYGKCARCAYTCGGVCTC |
| | CATGGCG |
| | (SEQ ID NO: 750) |
| Perissodactyla | TGAGCTTCCCTCCGCYCTMYRRRGV |
| Alignment | ARRVMDGNCNMHHRGCDGCMTTTAT |
| consensus | AAGACTCACAKRTCTRAAGMCATTT |
| sequence | MACRRWTAGGGTGACTTCCCACARK |
| 95%_Identity | VCACAGCRAYATGCAAAYATMGYGG |
| | RRYGYRCYTYYCCWGTMYCBKGYRG |
| | GYATCTNNNCKCCTRSACGCACGCG |
| | CRCCGSGTGTTCCCGCSCTGTGWCK |
| | CTAGGYRRGCSHTTCMYGRGRGRGR |
| | GKTGATGAYGKCARCMYTCGGVCTC |
| | MATGGCG |
| | (SEQ ID NO: 751) |
| Perissodactyla | TGAGCTTCCCTCCGCYCTMYRRRGV |
| Alignment | ARRVMDGNCNMHHRGCDGCMTTTAT |
| consensus | AAGACTCACAKRTCTRAAGMCATTT |
| sequence | MACRRWTAGGGTGACTTCCCACARK |
| 99% Identity | VCACAGCRAYATGCAAAYATMGYGG |
| | RRYGYRCYTYYCCWGTMYCBKGYRG |
| | GYATCTNNNCKCCTRSACGCACGCG |
| | CRCCGSGTGTTCCCGCSCTGTGWCK |
| | CTAGGYRRGCSHTTCMYGRGRGRGR |
| | GKTGATGAYGKCARCMYTCGGVCTC |
| | MATGGCG |
| | (SEQ ID NO: 752) |
| Perissodactyla | TGAGCTTCCCTCCGCYCTMYRRRGV |
| Alignment | ARRVMDGNCNMHHRGCDGCMTTTAT |
| consensus | AAGACTCACAKRTCTRAAGMCATTT |
| sequence | MACRRWTAGGGTGACTTCCCACARK |
| 100%_Identity | VCACAGCRAYATGCAAAYATMGYGG |
| | RRYGYRCYTYYCCWGTMYCBKGYRG |
| | GYATCTNNNCKCCTRSACGCACGCG |
| | CRCCGSGTGTTCCCGCSCTGTGWCK |
| | CTAGGYRRGCSHTTCMYGRGRGRGR |
| | GKTGATGAYGKCARCMYTCGGVCTC |
| | MATGGCG |
| | (SEQ ID NO: 753) |

In some embodiments, the promoter comprises a nucleotide sequence having at least 85%, 90%, 95%, 96%, 97%, 98%, 99%, or 100% identity to nucleotides 20-250 of any one of SEQ ID NOs: 748-753 or a functional fragment or variant (e.g., codon optimized) thereof.

### Primate H1 Promoters

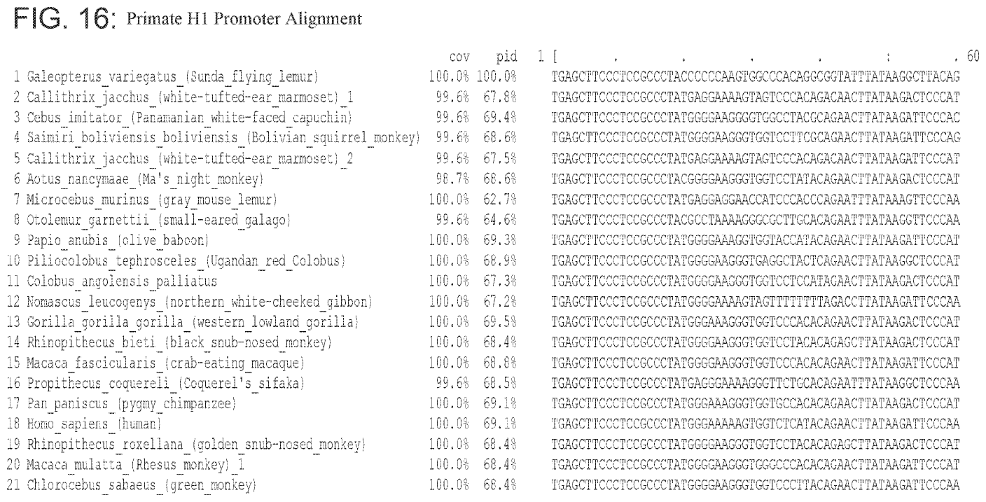

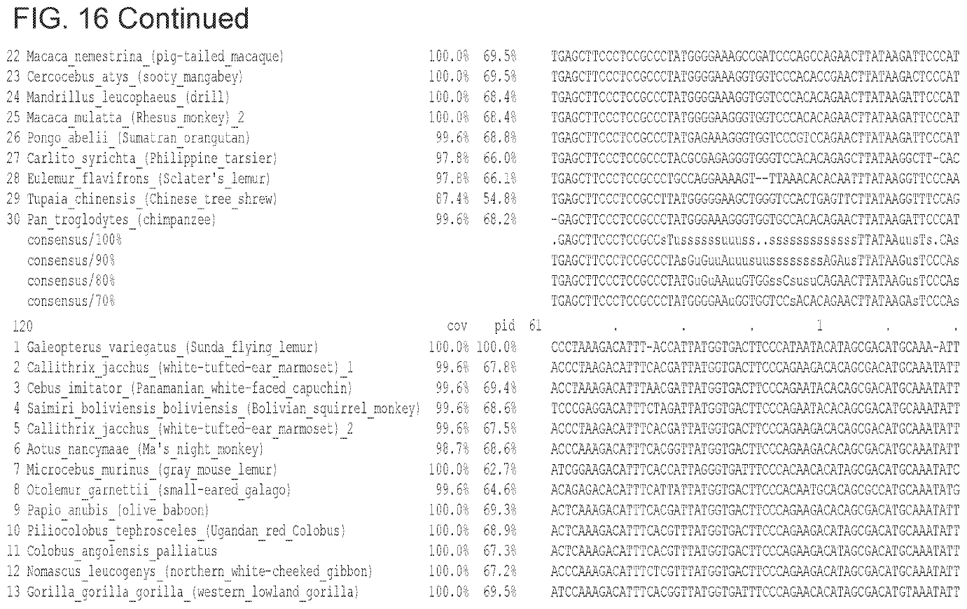

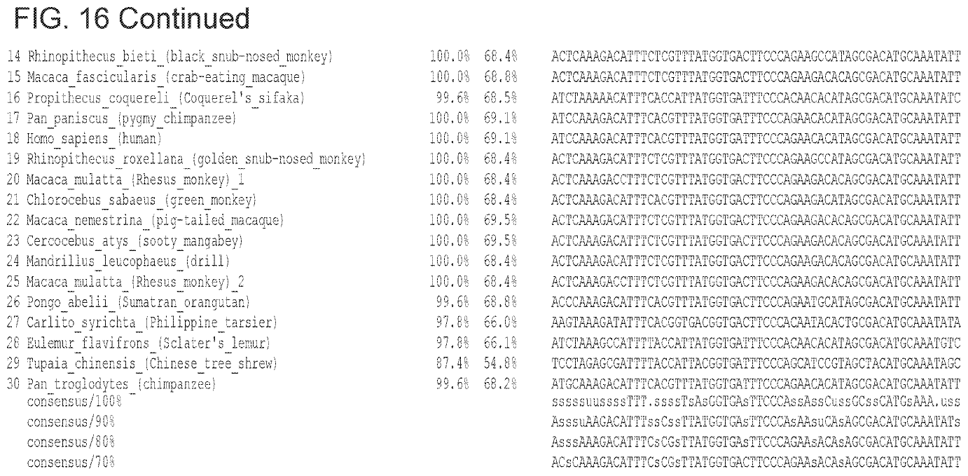

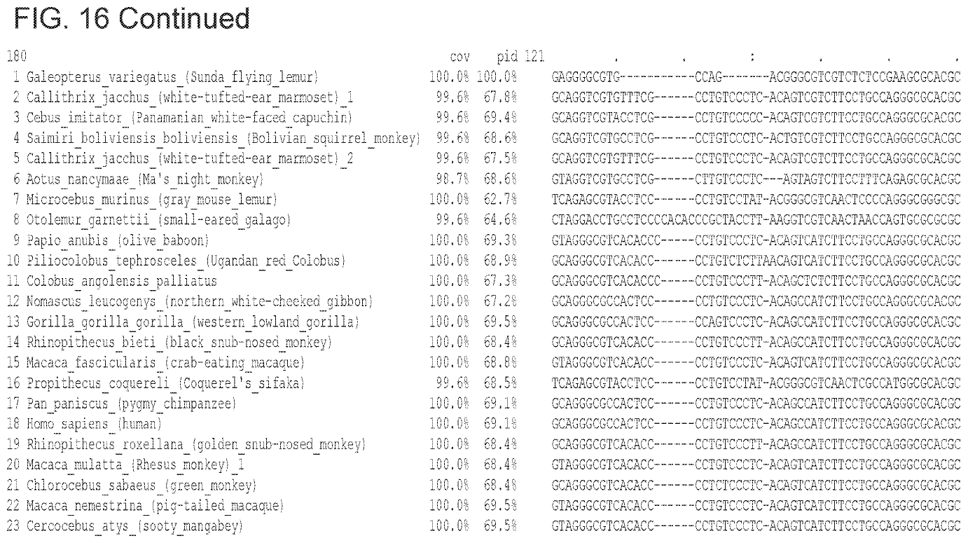

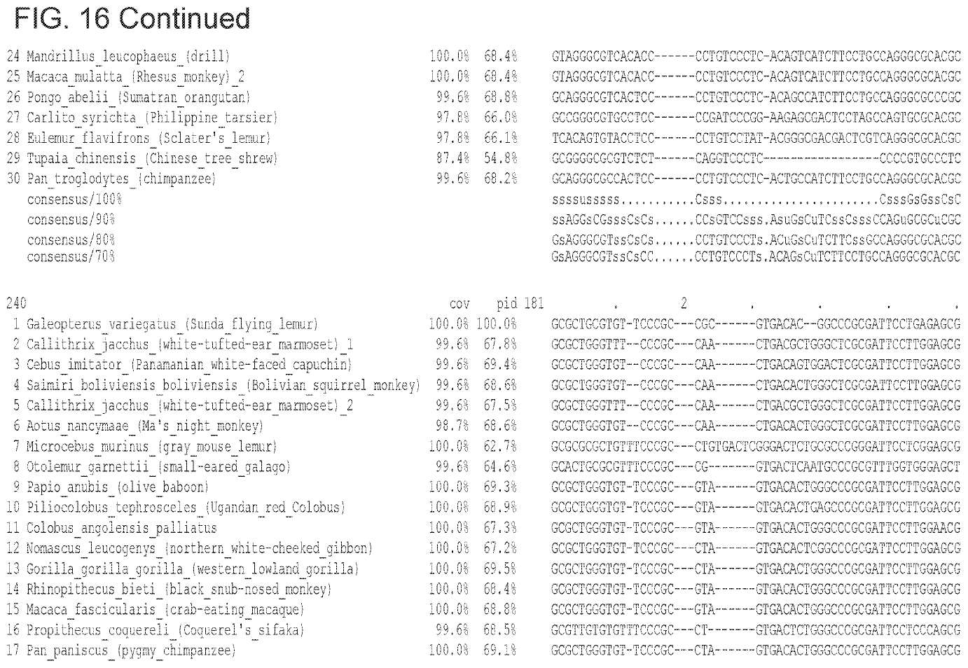

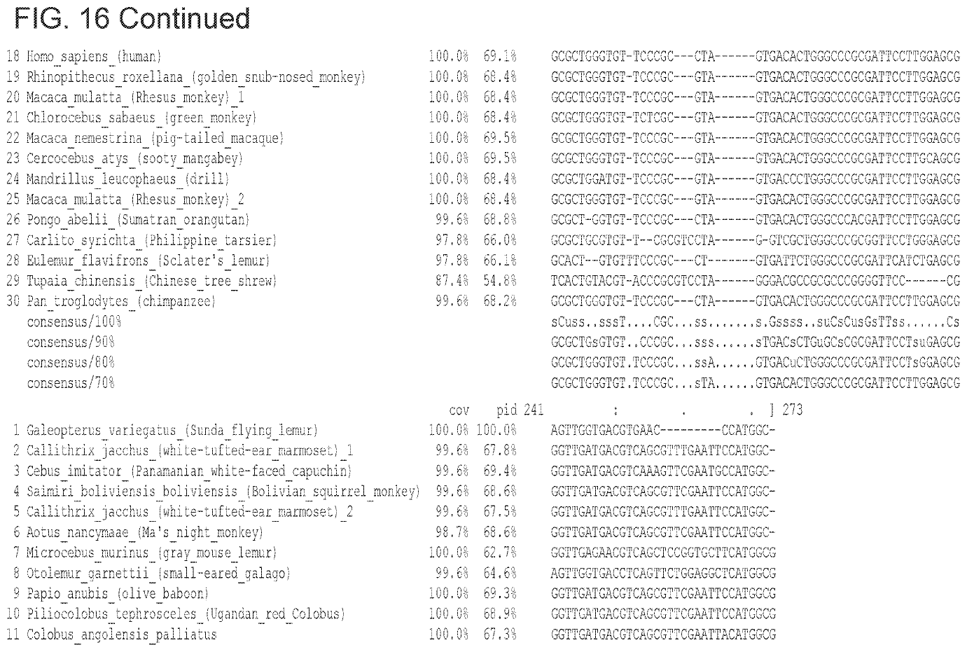

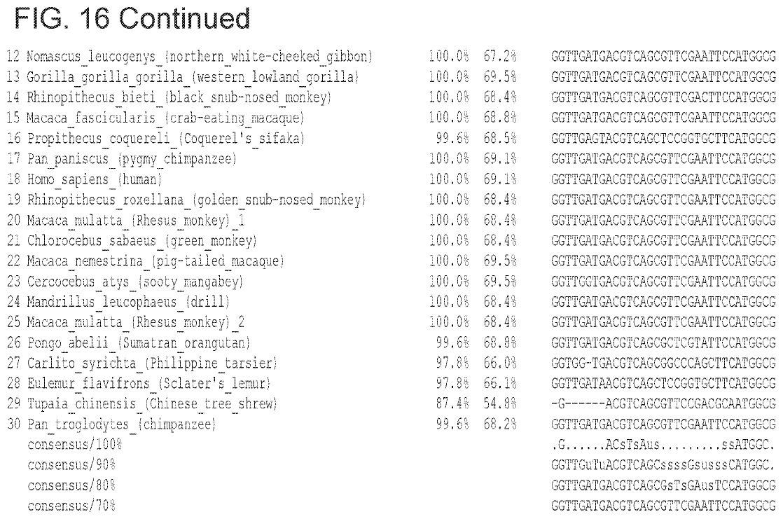

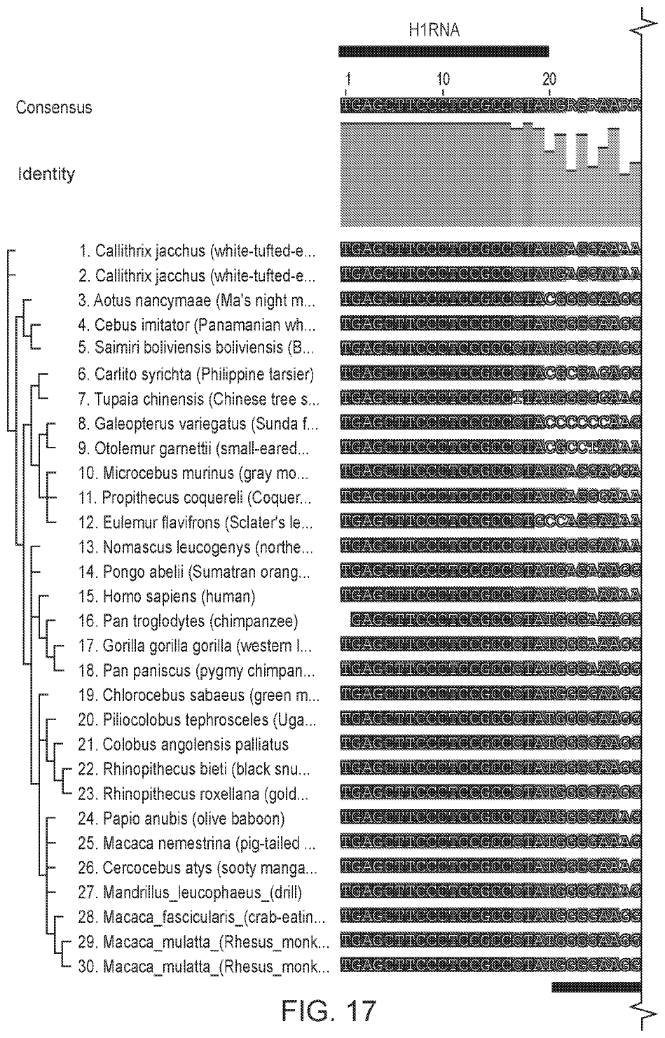

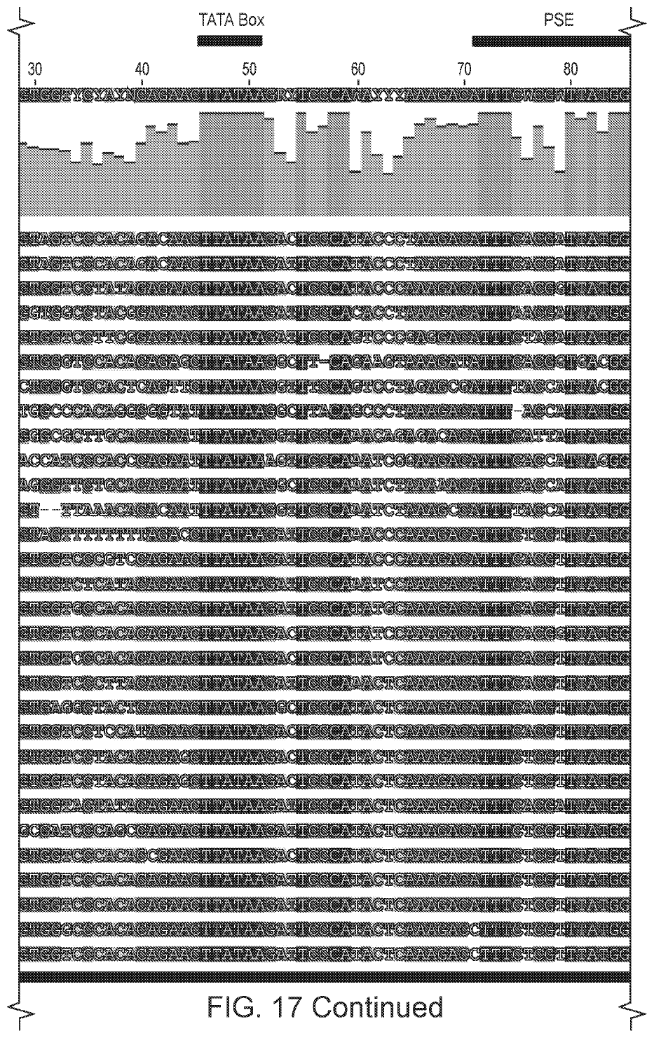

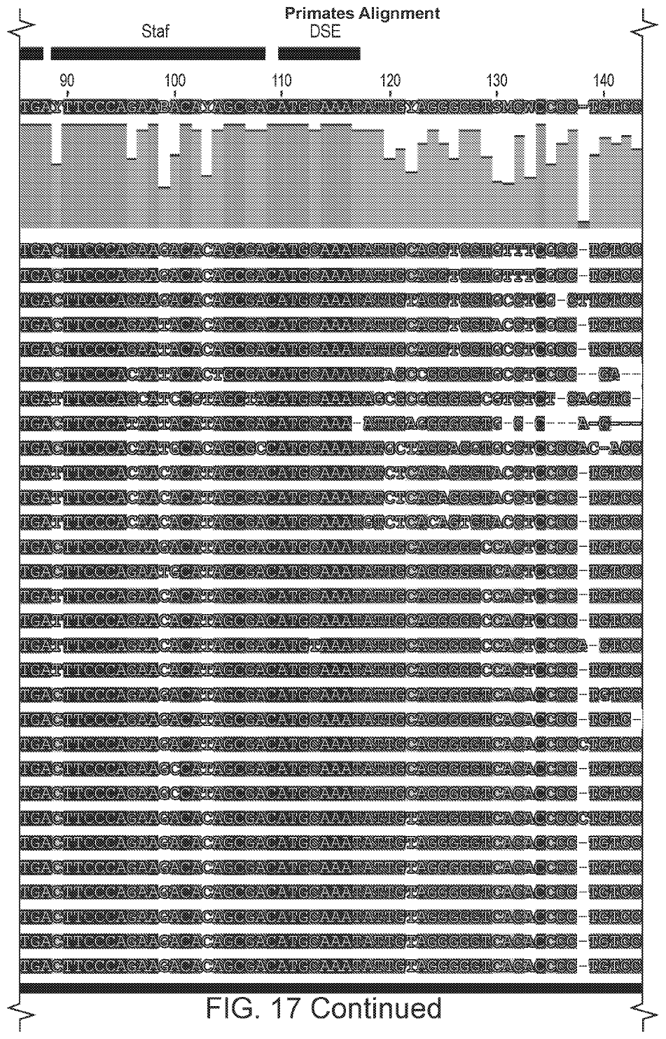

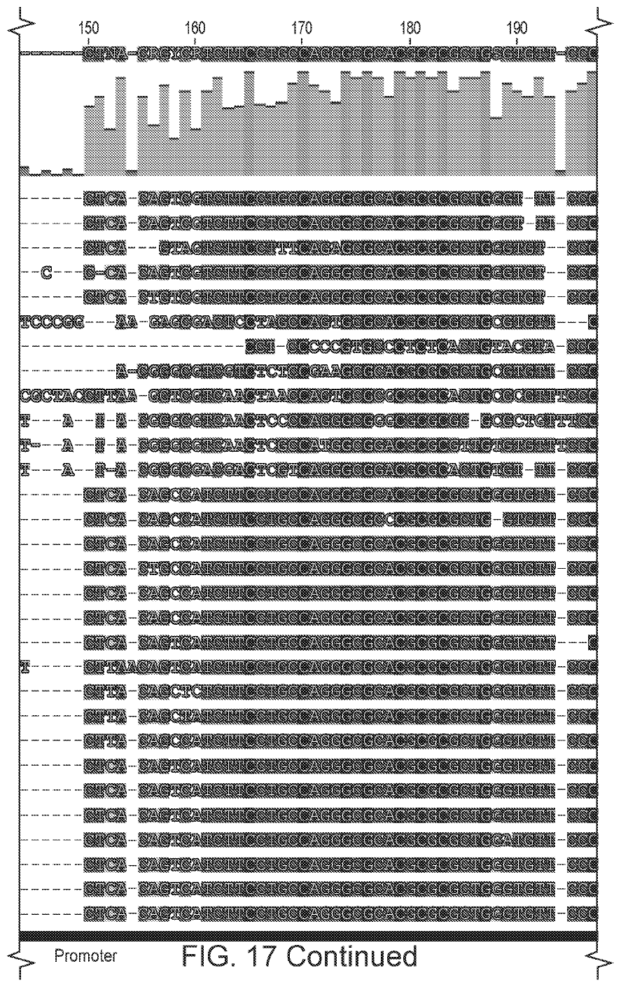

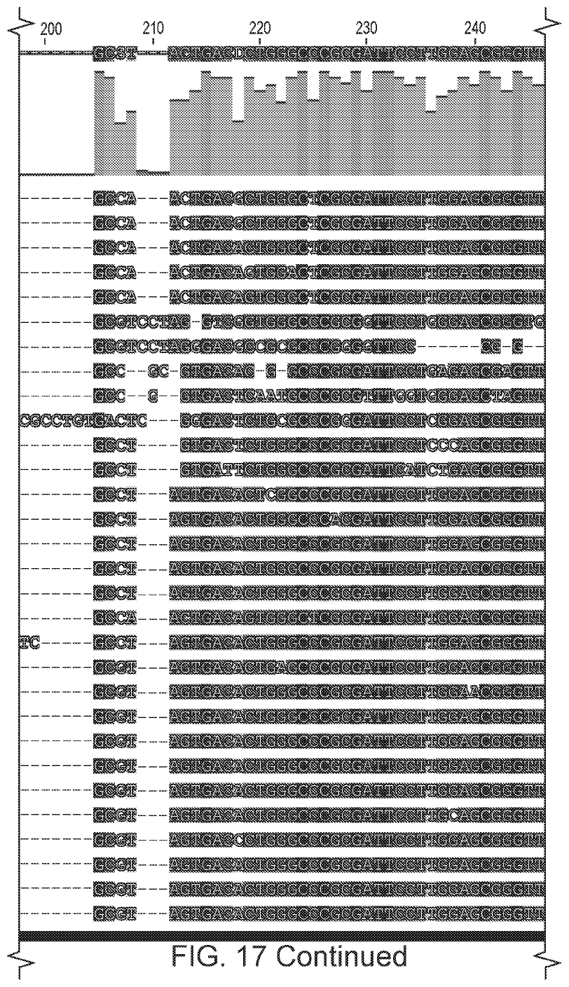

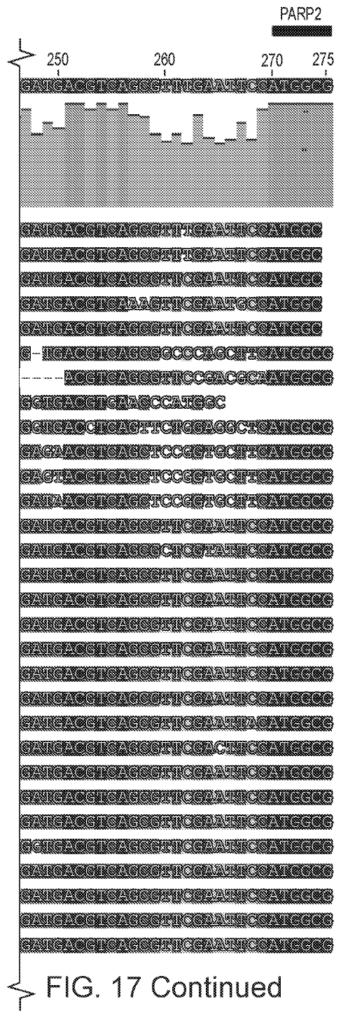

In certain embodiments, the promoter comprises a Primate H1 promoter. An alignment of Primate H1 promoter sequences is provided in FIG. 16 (wherein sequences numbered 1-30 in FIG. 16 correspond to SEQ ID NOs: 754-783, and consensus/100%, consensus/90%, consensus/80% and consensus/70% correspond to SEQ ID NOs: 1855-1858, respectively). FIG. 17 provides an second alignment of H1 promoter sequences from Primate species showing the TATA box, PSE, Staf, and DSE binding sites. Sequences numbered 1-30 in the alignment correspond to SEQ ID NOs: 755, 758, 759, 756, 757, 780, 783, 754, 761, 760, 769, 781, 765, 779, 771, 783, 766, 770, 774, 763, 764, 767, 772, 762, 775, 776, 777, 768, 773, and 788, respectively. The consensus sequence shown in FIG. 17 corresponds to SEQ ID NO: 1868. In some embodiments, the promoter comprises a nucleotide sequence having at least 85%, 90%, 95%, 96%, 97%, 98%, 99%, or 100% identity to nucleotides 20-267 of any one of the sequences in FIG. 16 or FIG. 17 or a functional fragment or variant (e.g., codon optimized) thereof. In certain embodiments, a functional fragment of a primate H1 promoter comprises at least a TATA box, or a PSE, Staf, or DSE binding site.

In certain embodiments, the Primate H1 promoter comprises a sequence selected from those in TABLE 14.

TABLE 14

| | |
|---|---|
| Primate | TGAGCTTCCCTCCGCCCTATGRGRA |
| Alignment | ARRGTGGTYCYAYNCAGAACTTATA |
| consensus | AGRYTCCCAWAYYYAAAGACATTTC |
| sequence | WCGWTTATGGTGAYTTCCCAGAABA |
| 75%_Identity | CAYAGCGACATGCAAATATTGYAGG |
| | GCGTSMCWCCCCTGTCCCTYACRGY |
| | CRTCTTCCTGCCAGGGCGCACGCGC |
| | GCTGGGTGTTCCCGCSTAGTGACDC |
| | TGGGCCCGCGATTCCTTGGAGCGGG |
| | TTGATGACGTCAGCGTTCGAATTCC |
| | ATGGCG |
| | (SEQ ID NO: 784) |
| Primate | TGAGCTTCCCTCCGCCCTAYGRGRA |
| Alignment | ARRVKRRKYYYDYNSAGARYTTATA |
| consensus | AGRYTCCCADAYYYAAAGACATTTC |
| sequence | WCSWTTATGGTGAYTTCCCASAABM |
| 85%_Identity | CAYAGCGACATGCAAATATYGYAGG |
| | KCGYSMCWCSCCKGTCCCWYACRGB |
| | CRTCWWCYYKCCAGDGCGCACGCGC |
| | GCTGSGTGTNCCCGCSWNSTGACDC |
| | TGGGCYCGCGATTCCTBGGAGCGGG |
| | TTGRTGACGTCAGCKYYSGWRYTYC |
| | ATGGCG |
| | (SEQ ID NO: 785) |
| Primate | TGAGCTTCCCTCCGCCCTAYGRGRR |
| Alignment | ARRVKRRKBYYDYNSAGARYTTATA |
| consensus | AGRYTCCCADAYYYDAAGACATTTY |
| sequence | WCSWTTATGGTGAYTTCCCASAABM |
| 90%_Identity | CAYAGCGACATGCAAATATYKYAGG |
| | KCGYVHCWCSCCKGTCCYWYANRGB |
| | CRTCWWCYYKCCAGDGCGCVCGCGC |
| | GCTGSGTGTNNCCCGCSWNSTGACD |
| | CTGSGCYCGCGATTCCTBNGAGCGG |
| | GTTGRTRACGTCAGCKYYSGWRYKY |
| | CATGGCG |
| | (SEQ ID NO: 786) |
| Primate | TGAGCTTCCCTCCGCCCTAYSVSNR |
| Alignment | ARRVBNVKBHYDBNBVSWNYTTATA |
| consensus | AGRYTYNCANWYBBDRAVMBMTTTN |
| sequence | WHSDTTAYGGTGAYTTCCCASAABV |
| 95%_Identity | CAYAGCGACATGCAAATATNKYRGR |
| | KCGYVHYWCNNCHDSTNNYNNNNDN |
| | BNNWCDNCYHNYCVNDGCGCVCGCG |
| | CRCTNBRYKTNNCNCGCNNNSDNSK |
| | GACDCNNNGCYCGSGRTTCVTBNSA |
| | NCGRGTNGNKNACGTCARHKNYBSN |
| | NNNYCATGGCG |
| | (SEQ ID NO: 787) |
| Primate | TGAGCTTCCCTCCGCCYTRYSVSNV |
| Alignment | RRRNBNNBNHHNBNBVSWNYTTATA |
| consensus | ARRYTYNCANHHNBDRRVMBMTTTN |
| sequence | WHBDTKABGGTGAYTTCCCABMABV |
| 99%_Identity | CRYWGCKMCATGYAAANRKNBHVSR |
| | DYSYVNNNNNNNNNNNCHDVNNNNN |
| | NNNNNNNNNNNNNNNNNNCVNNGYG |
| | SVCKCKCRYKNNVYKTNNNNCGCNN |
| | NSDNNNNNNNSNGWYNSNNNRCYCR |
| | SGDTTSVNNNNNNCKNGNNNNNNAC |
| | STSARHNNNNNNNNNHMATGGCG |
| | (SEQ ID NO: 788) |
| Primate | TGAGCTTCCCTCCGCCYTRYSVSNV |
| Alignment | RRRNBNNBNHHNBNBVSWNYTTATA |
| consensus | ARRYTYNCANHHNBDRRVMBMTTTN |
| sequence | WHBDTKABGGTGAYTTCCCABMABV |
| 100%_Identity | CRYWGCKMCATGYAAANRKNBHVSR |
| | DYSYVNNNNNNNNNNNCHDVNNNNN |
| | NNNNNNNNNNNNNNNNNNCVNNGYG |
| | SVCKCKCRYKNNVYKTNNNNCGCNN |
| | NSDNNNNNNNSNGWYNSNNNRCYCR |
| | SGDTTSVNNNNNNCKNGNNNNNNAC |
| | STSARHNNNNNNNNNHMATGGCG |
| | (SEQ ID NO: 789) |

In some embodiments, the promoter comprises a nucleotide sequence having at least 85%, 90%, 95%, 96%, 97%, 98%, 99%, or 100% identity to nucleotides 20-250 of any one of SEQ ID NOs: 784-789 or a functional fragment or variant (e.g., codon optimized) thereof.

### Rodent H1 Promoters

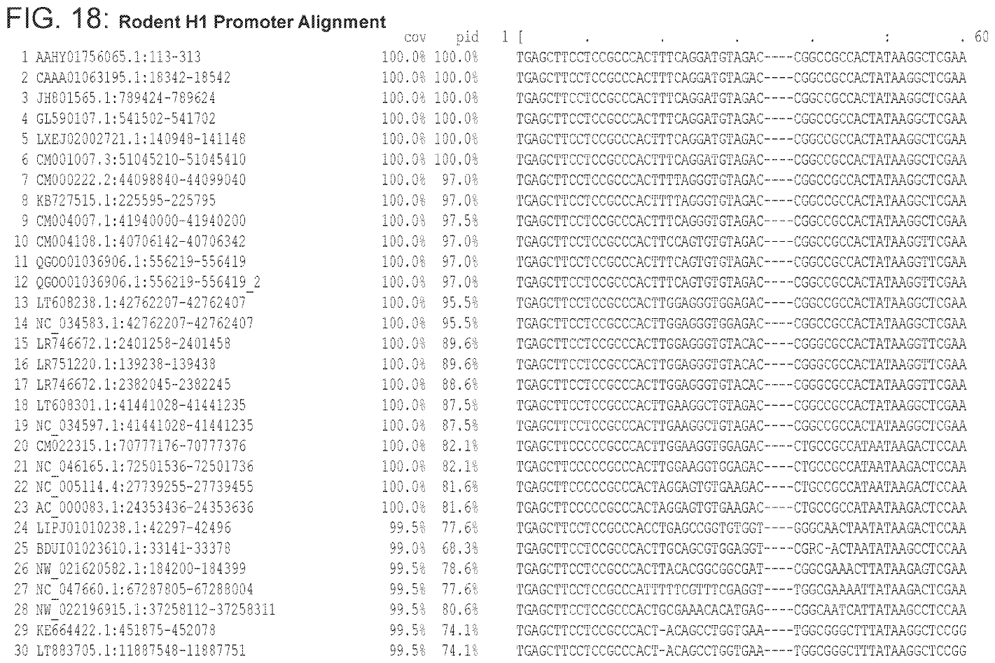

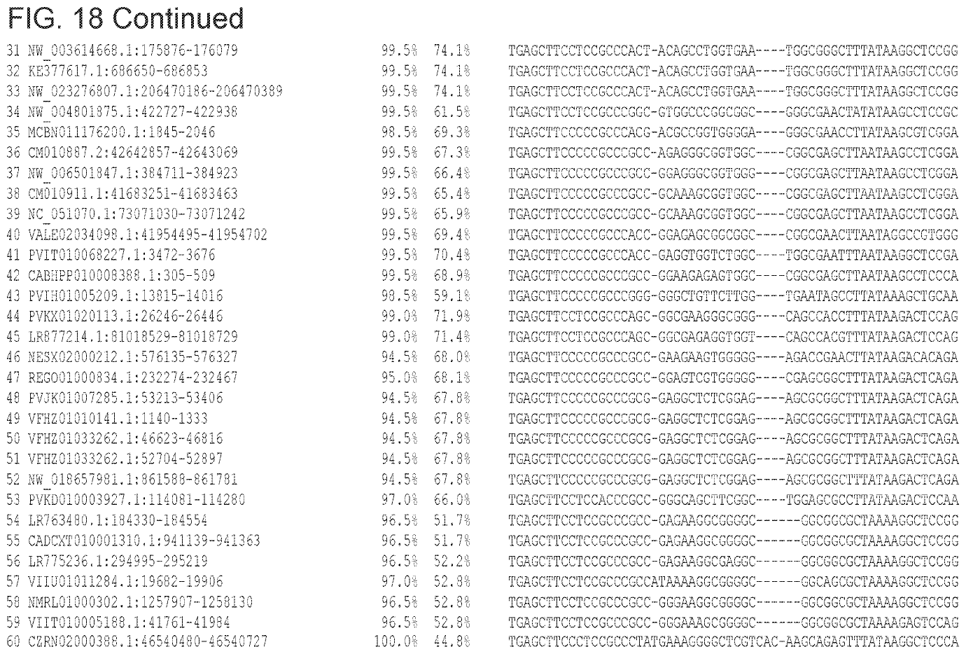

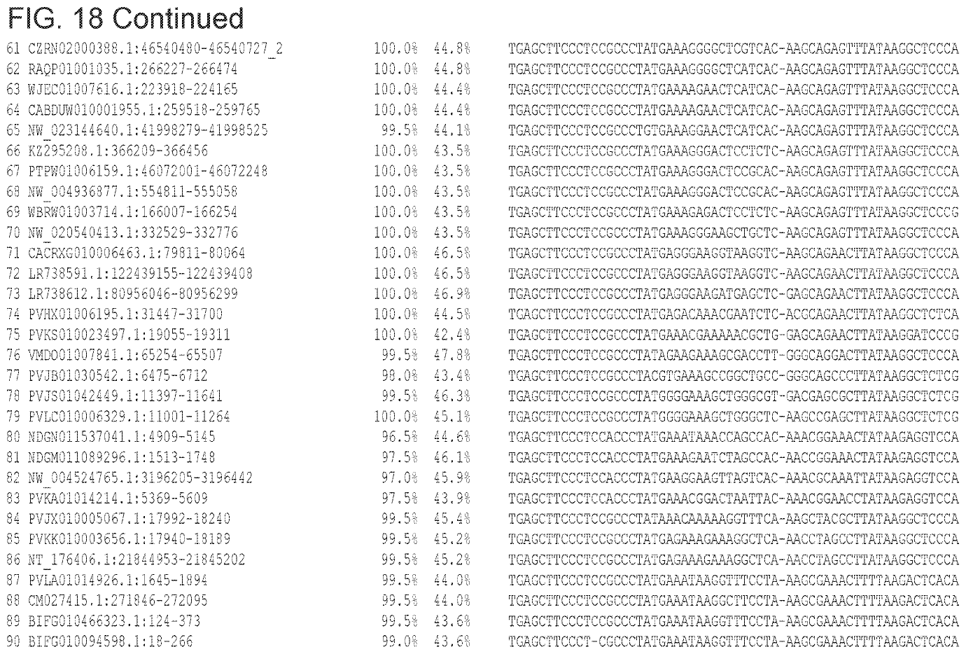

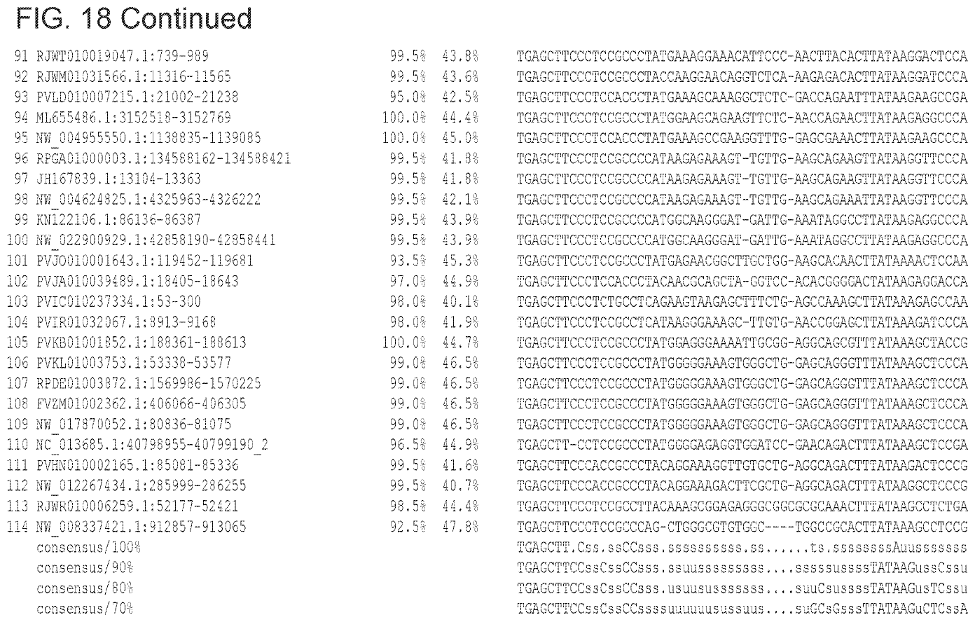

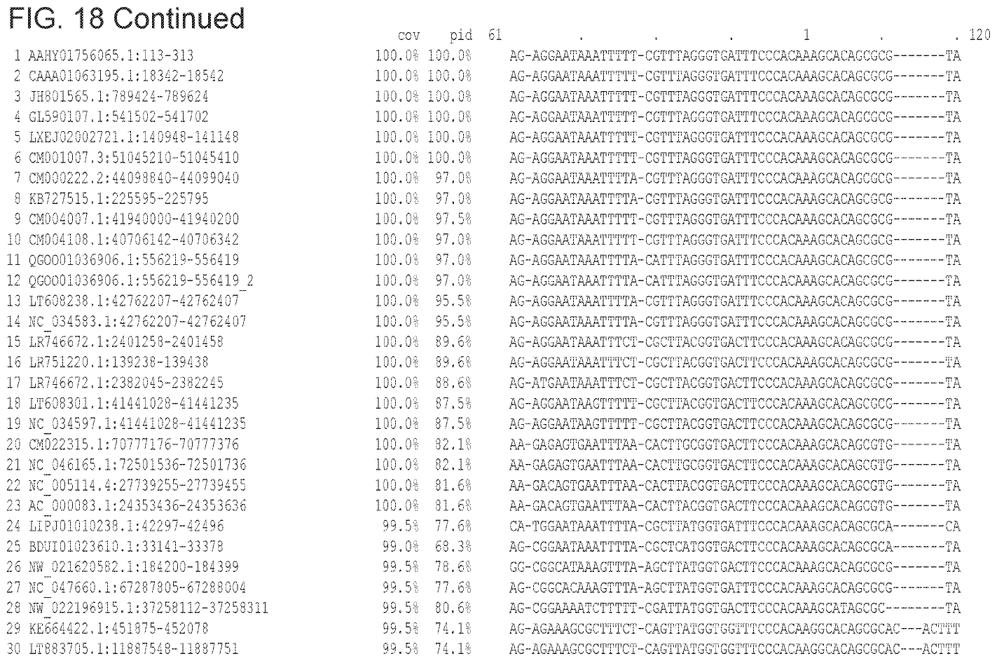

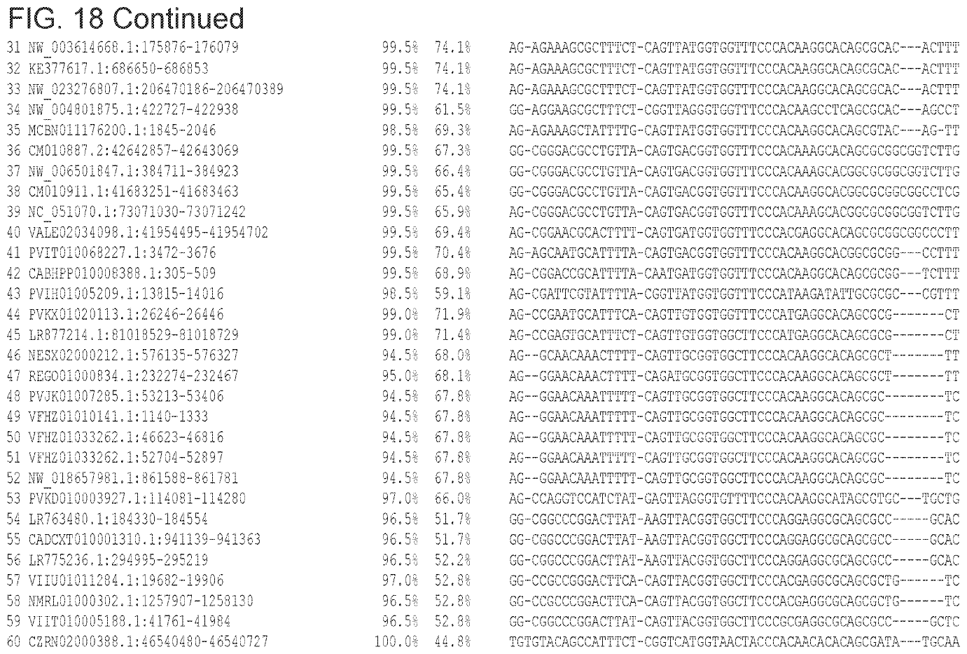

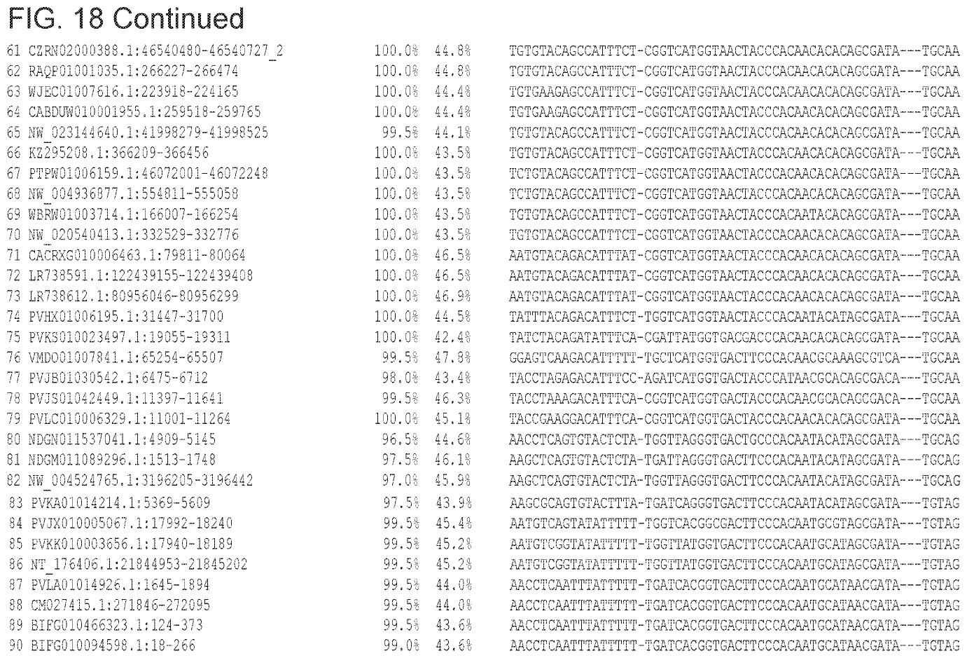

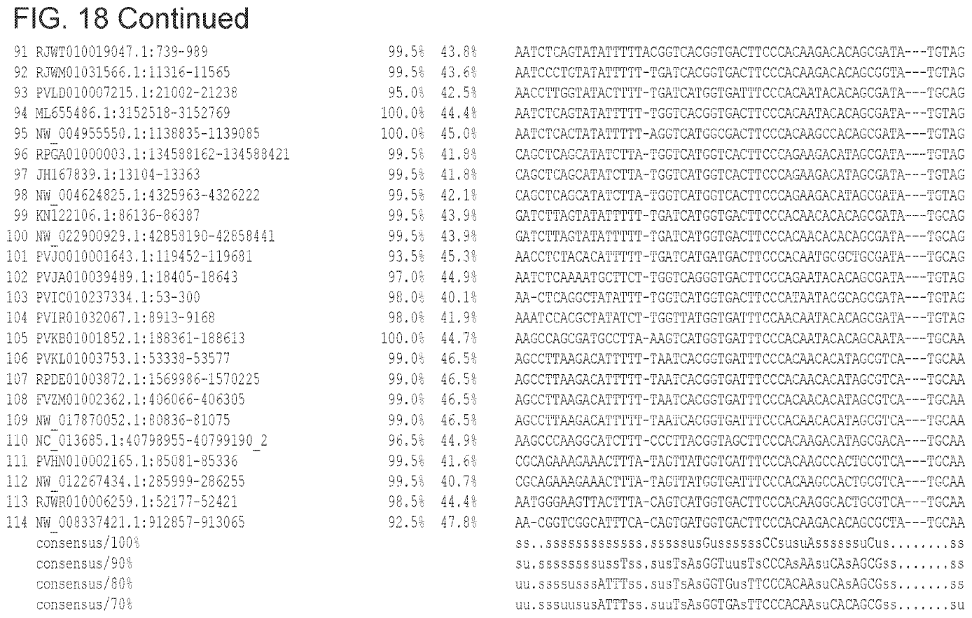

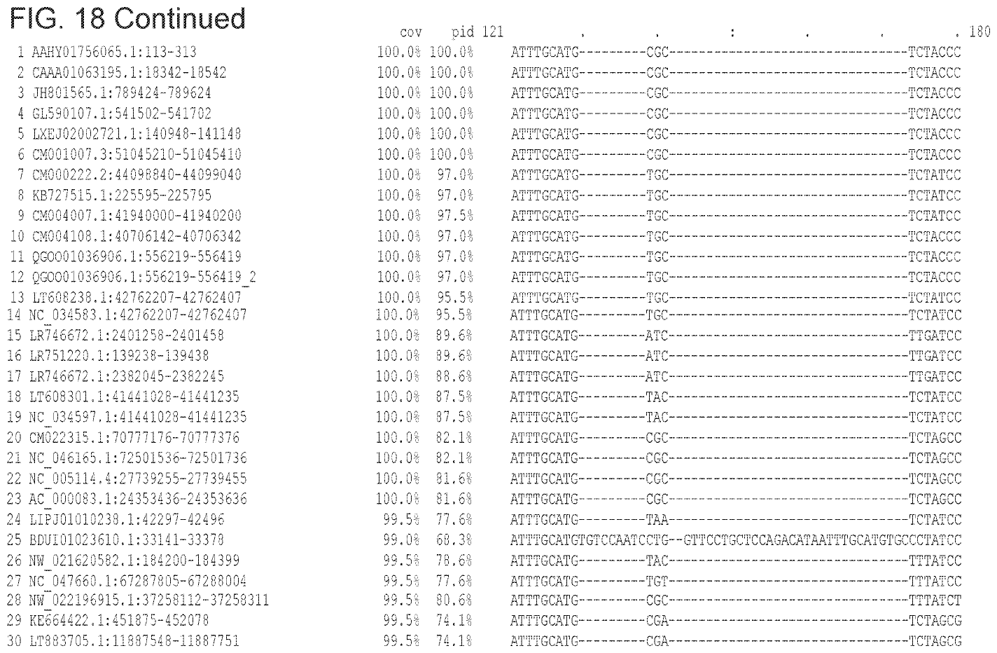

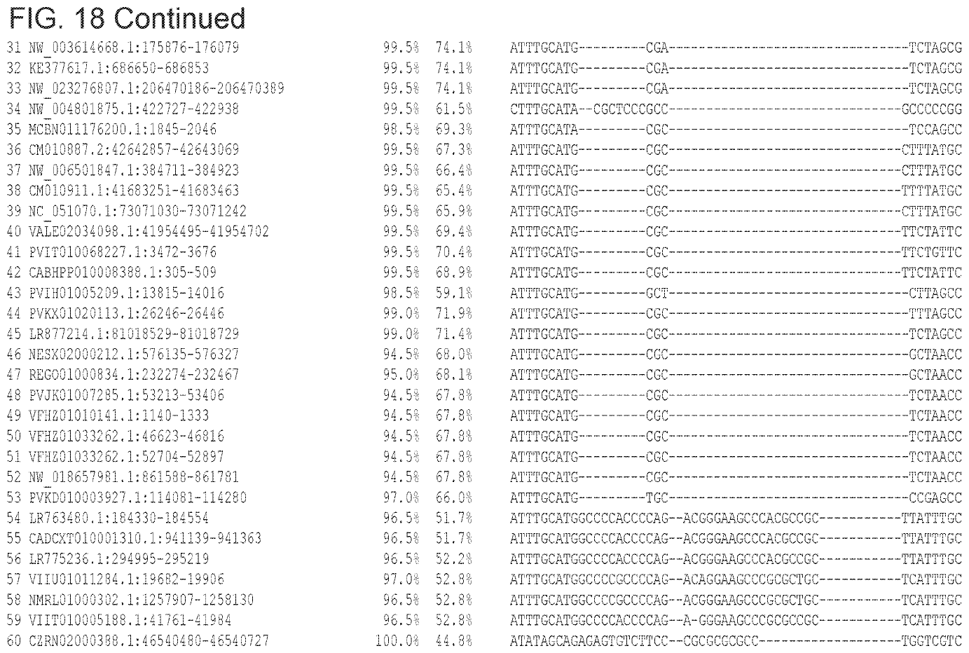

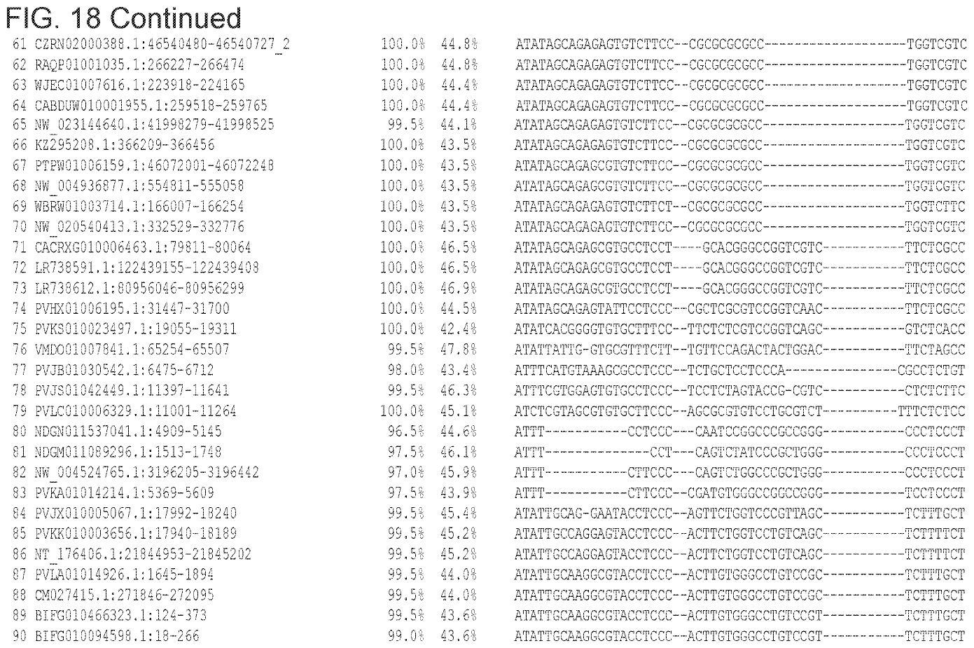

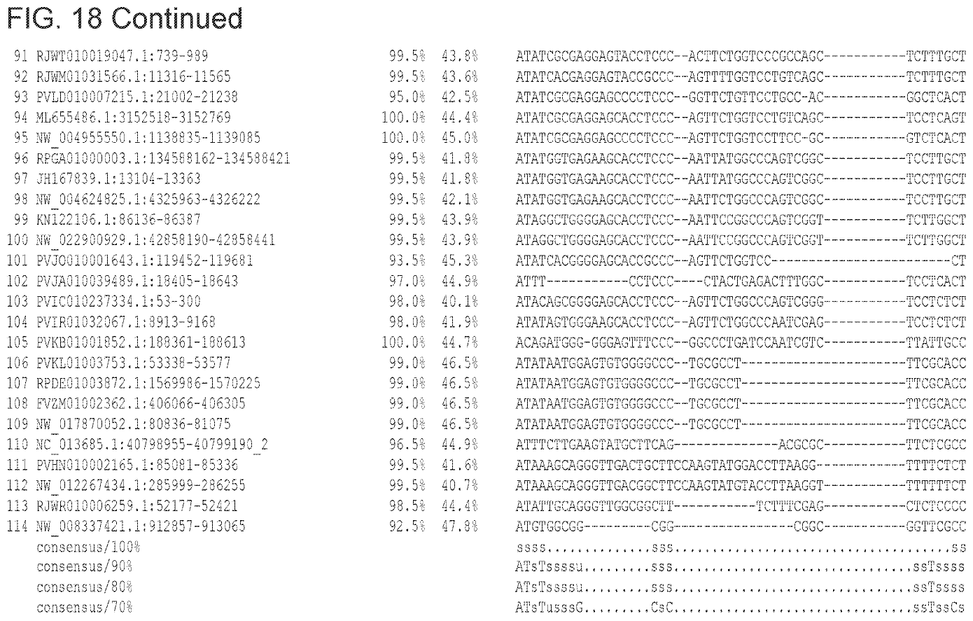

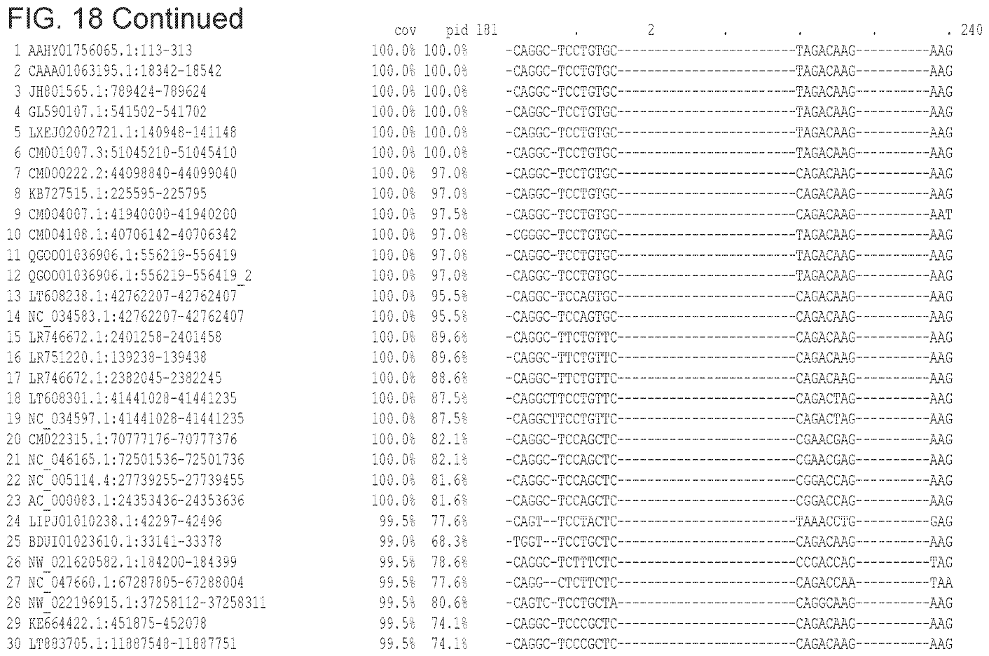

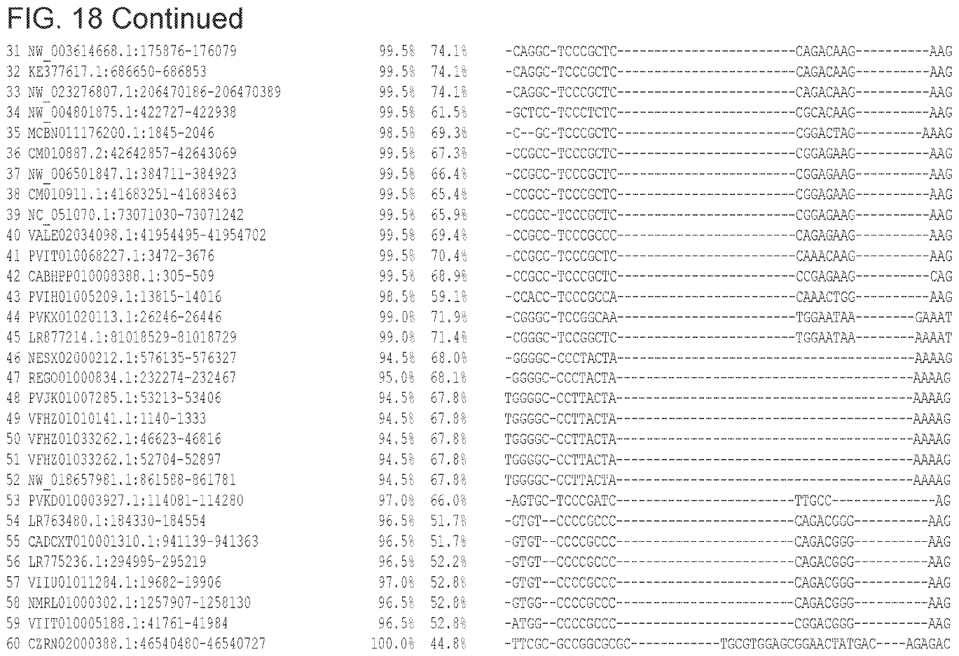

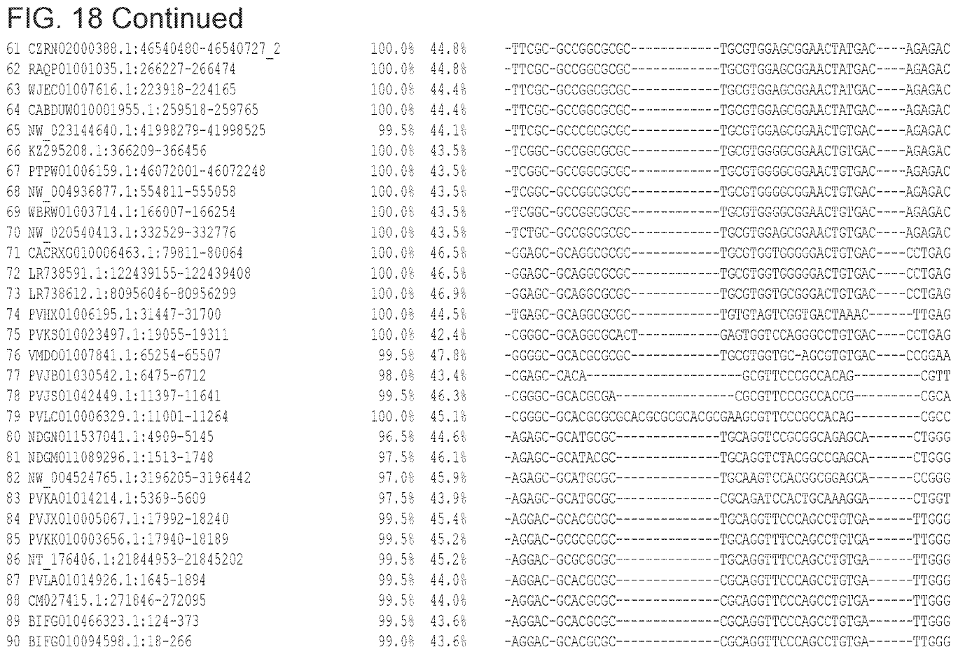

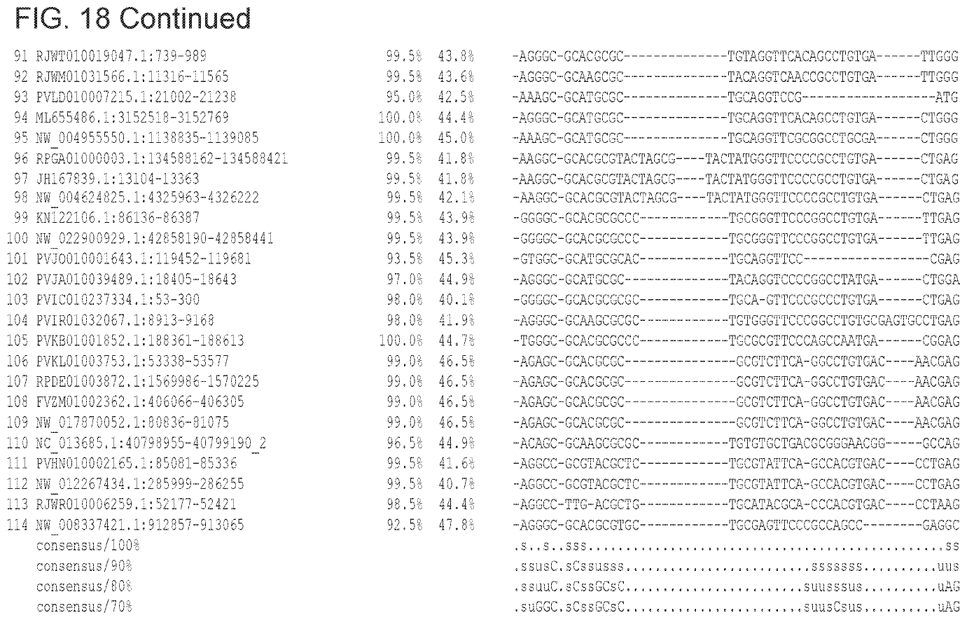

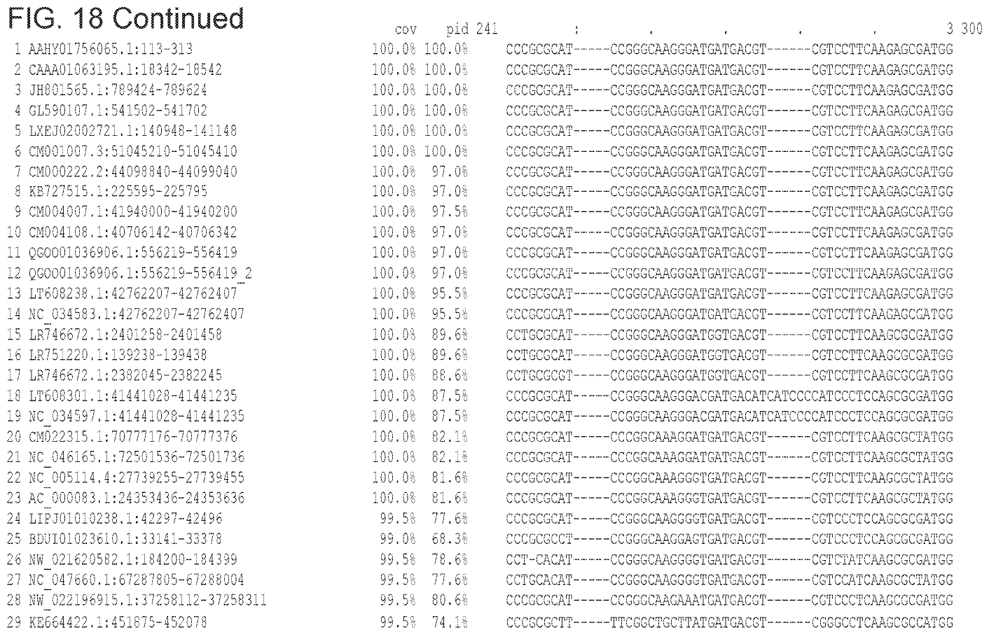

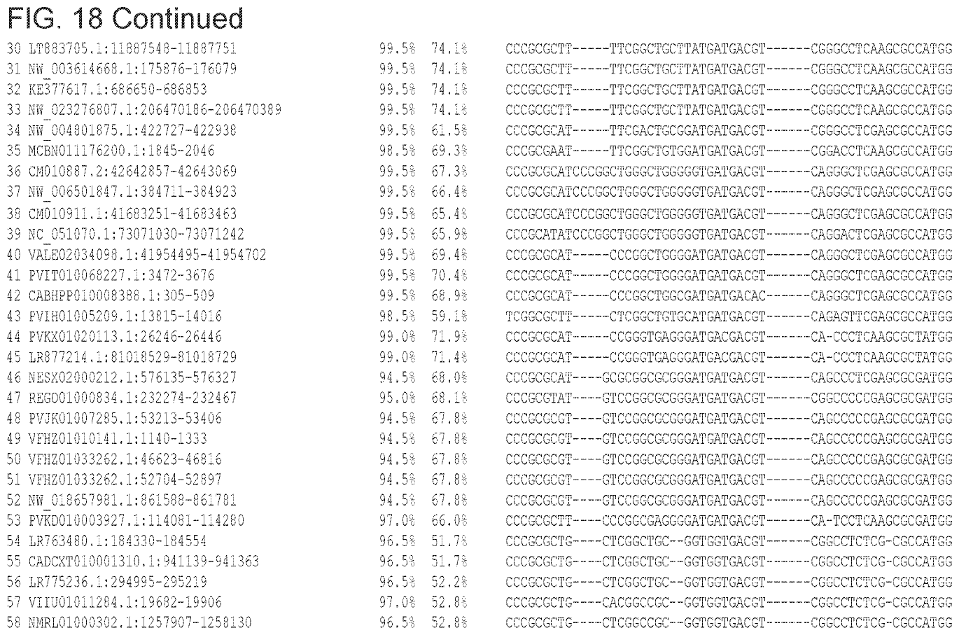

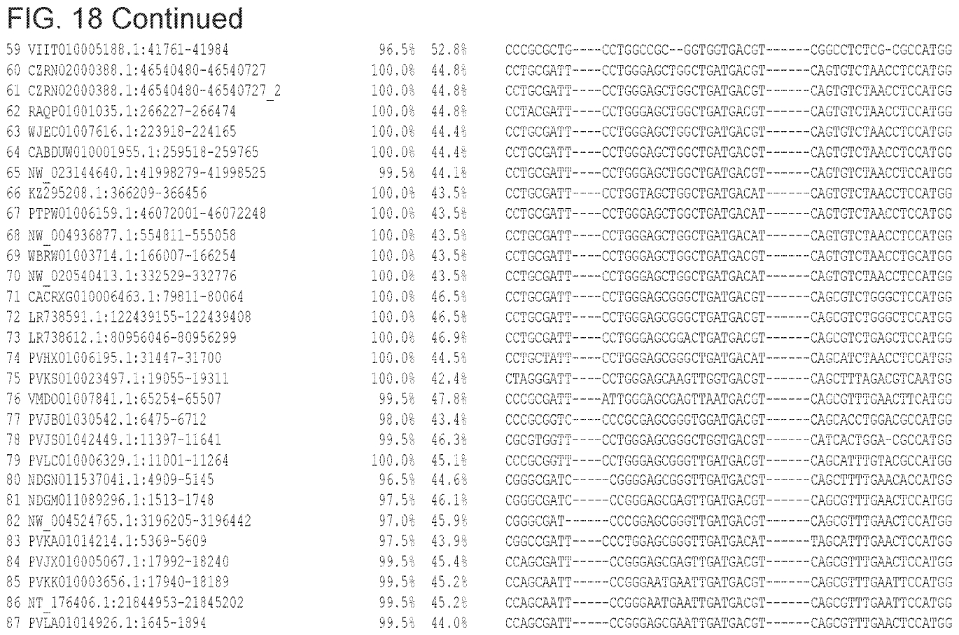

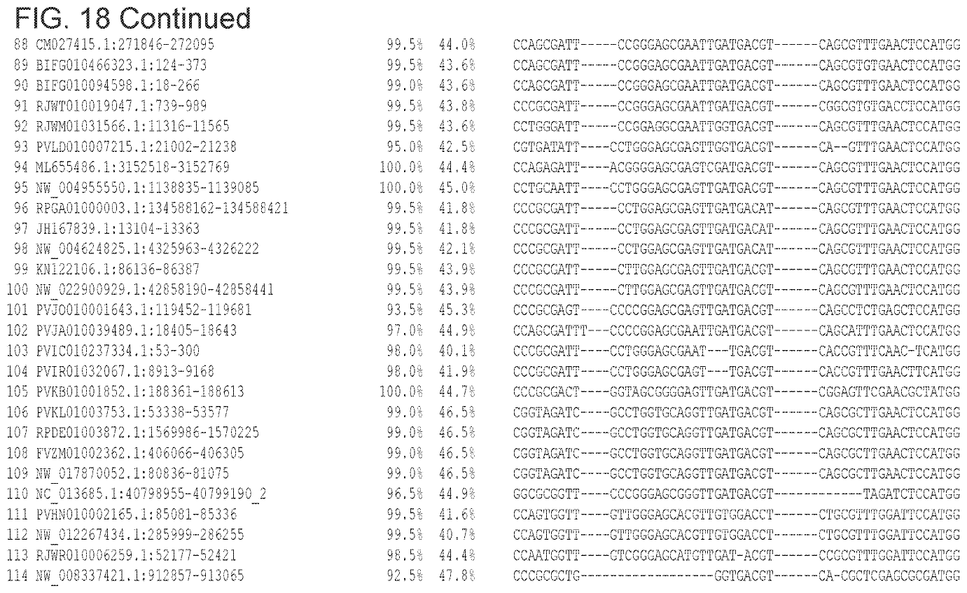

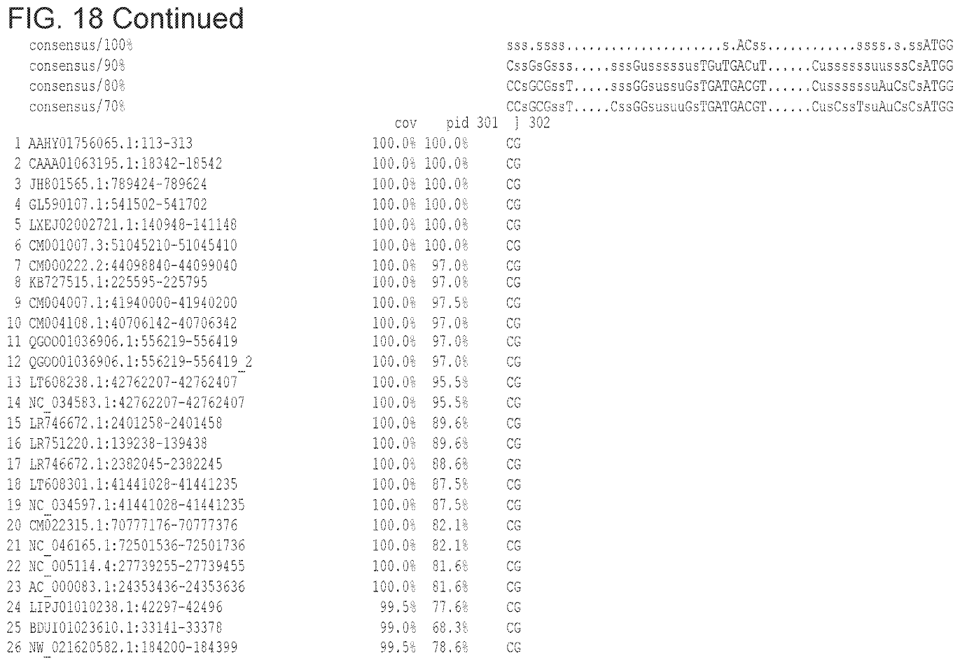

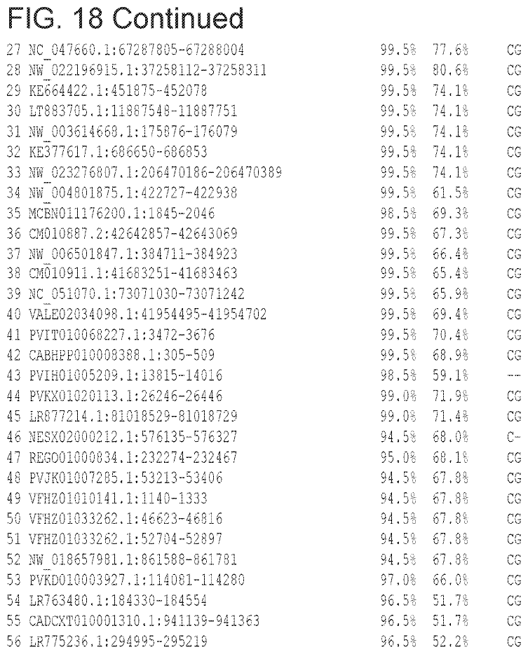

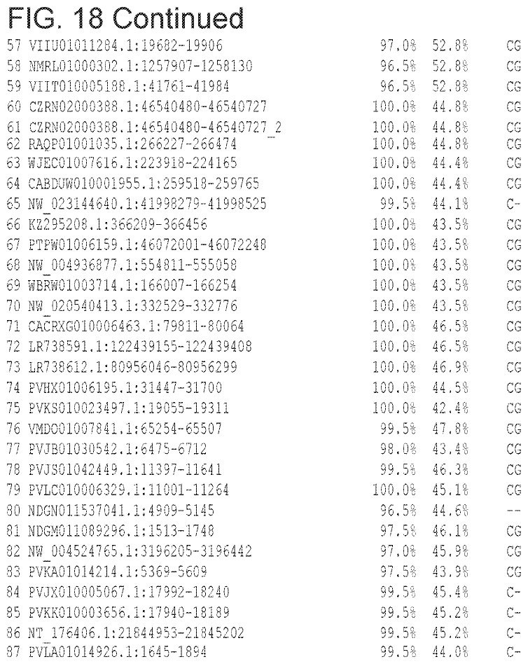

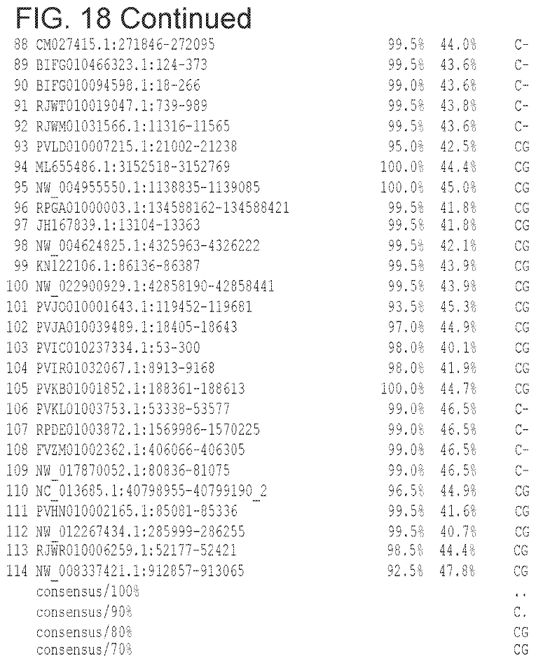

In certain embodiments, the promoter comprises a Rodent H1 promoter. An alignment of Rodent H1 promoter sequences is provided in FIG. 18 (wherein sequences numbered 1-114 in FIG. 18 correspond to SEQ ID NOs: 790-903 or 1859, and consensus/100%, consensus/90%, consensus/80% and consensus/70% correspond to SEQ ID NOs: 1860-1863, respectively). In some embodiments, the promoter comprises a nucleotide sequence having at least 85%, 90%, 95%, 96%, 97%, 98%, 99%, or 100% identity to nucleotides 20-296 any one of the sequences in FIG. 18 or a functional fragment or variant (e.g., codon optimized) thereof.

In certain embodiments, the Rodent H1 promoter a sequence selected from those in TABLE 15.

TABLE 15

| | |
|---|---|
| Rodent | TGAGCTTCCYYCSSCCMYHTRRRRV |
| Alignment | RDRBDSRBYWSCMRGCVRVMHYTAT |
| consensus | AAGRCTCSMAWRYMKVMRKRHATTT |
| sequence | YWAYRVTYAYGGTGRYTTCCCACAA |
| 75%_Identity | VRCACAGCGMKACGGTGYWRATWTR |
| | SMWGRGHGYRYCKYSCCCMSBKSBN |
| | GBCCDSYCVKSATTTGCATGTBTYY |
| | TMDCYTVRGGCTKCMYGCKCRCTAG |
| | CGCGCATACTGCRKGKYSMSRGMCW |
| | RKGACAGTGMNWRAGCCYGCGMWTC |
| | CCGSCYSGGMRMKRGNTGATGACGT |
| | CATCCCCRKCSYYYRARCKCSATGG |
| | CG |
| | (SEQ ID NO: 904) |
| Rodent | TGAGCTTCCYYCSSCCVYHTRVRRV |
| Alignment | VDDBDNDBYHVCVRSSVRVVHYTAT |
| consensus | AAGRSTCSVRDRBVKVMRBVHAYTT |
| sequence | YWAYRVTYABGGTRRYTWCCCACAA |
| 85%_Identity | NRCAYAGCGMBVCGGWSYWDATWTV |
| | SMDRRSHSYRYYKYVYCCHVBKVBN |
| | GBCCNBBYVKBATTTGCATGTBYYB |
| | THDYYTVVRSCTKCMBGYKCNCWMG |
| | CGCGCAYRCTGYRKRKHSMSRRMMD |
| | RKGACAGTGMNHRRSCCHGCGMWTY |
| | CCGSYYSGGMRVDRRNTGATGACGT |
| | CATCCCCRKSSYYYRARMKCSATGG |
| | CG |
| | (SEQ ID NO: 905) |
| Rodent | TGAGCTTCCYYCSSCCVYHYDVRRN |
| Alignment | VNDNDNDBYHVCVRSSVRVVHYTAT |
| consensus | AAGRBKCVVRDRBVBVVVBVNMYYT |
| sequence | HWAYRNTYABGGTRRYTWCCCASAA |
| 90% Identity | NRCAYAGCGHBVCGGWSYWDATWTV |
| | VHDRRSHNYRYYBYVBCCHVBBVNN |
| | NBCCNBBBVDBATTTGCATGTBYBB |
| | THNBYTNNRNCTBCMBRYKMNCWMG |
| | CGCGCAYRCYRYRBRKHSVBRRMMN |
| | RKSACAGTGMNHRRSCSHGMGMWBY |
| | CCGSYYSGGHDVDRRNTGRTGACRT |
| | CATCCCCRKBSYYYRRVMKCSATGG |
| | CG |
| | (SEQ ID NO: 906) |
| Rodent | TGAGCTTCCYYCSVCCVYNHDNVVN |
| Alignment | NNNNNNNNBNVCNDVNVRVVNYWAW |
| consensus | AARVNKYVVRNRBVNNVVBVNMYBT |
| sequence | HWAHRNTBRBGGTRRYTWCCCASRA |
| 95%_Identity | NRCRYWGCGHNVCGGHSYWNATWKN |
| | VHDRRVHNBNBBBYNNCCNVNBNNN |
| | NNNCNNNBNDBATTTGCATGTBBBN |
| | KHNBBTNNVNCTBYHNRYBMNCWMG |
| | CGCGCAYRCYRYRBVKNBVBVVMVN |
| | RDSMSAGTGMNHRRBCSNKHRVDBY |
| | CCGSYYBGSHDVNDDNTGRTGACRT |
| | CATCCCCRKBVYYYVRVHKCBATGG |
| | CG |
| | (SEQ ID NO: 907) |
| Rodent | TGAGCTTCCYHCNVCCNBNNNNVVN |
| Alignment | NNNNNNNNBNNCNNVNNVVNNHWWW |
| consensus | AARVNBHNVRNVNNNNNVNNNVBNY |
| sequence | HNAHRNTBRBGGYVRYTWCCCABRA |
| 99%_Identity | NVCRYDRCGHNVCGGHSYHNATNDN |
| | NHNRNVNNNNNBBNNNCCNNNNNNN |
| | NNNHNNNNNNNATTTGCATGTBBBN |
| | BNNBBTNNNNCTBYNNDYBHNSWMG |
| | CGCGCAYRCBRNDNVBNNVBNVVVN |
| | VNVVSAGTGMNNNNNBSNDNDNNBY |
| | CCGVNBBGVNDNNNDNYGDBGACVT |
| | CATCCCCDBNNHBHVRVHKYBATGG |
| | CG |
| | (SEQ ID NO: 908) |
| Rodent | TGAGCTTCCYHCNVCCNNNNNNVNN |
| Alignment | NNNNNNNNBNNCNNVNNVNNNHWWW |
| consensus | ARRVNNNNVVNVNNNNNNNNNVBNY |
| sequence | HNANVNWBRBGRYVDYKDCCMRBRA |
| 100%_Identity | NVYDHDRCRNNVCGGHSYHNMYNNN |
| | NNNDNVNNNNNBBNNNCCNNNNNNN |
| | NNNHNNNNNNNATTTGCATGTBBBN |
| | BNNBBTNNNNCTBHNNDHNHNSWMG |
| | CGCGCAYRCBRNDNVBNNVBNVVVN |
| | NNVVSAGTGMNNNNNBBNNNDNNBY |
| | CCGVNBNSNNDNNNNNBRDBGACVY |
| | CATCCCYNBNNHBNVDNNDBNATGG |
| | CG |
| | (SEQ ID NO: 909) |

In some embodiments, the promoter comprises a nucleotide sequence having at least 85%, 90%, 95%, 96%, 97%, 98%, 99%, or 100% identity to nucleotides 20-296 of any one of SEQ ID NOs: 904-909 or a functional fragment or variant (e.g., codon optimized) thereof.

### Xenarthra H1 Promoters

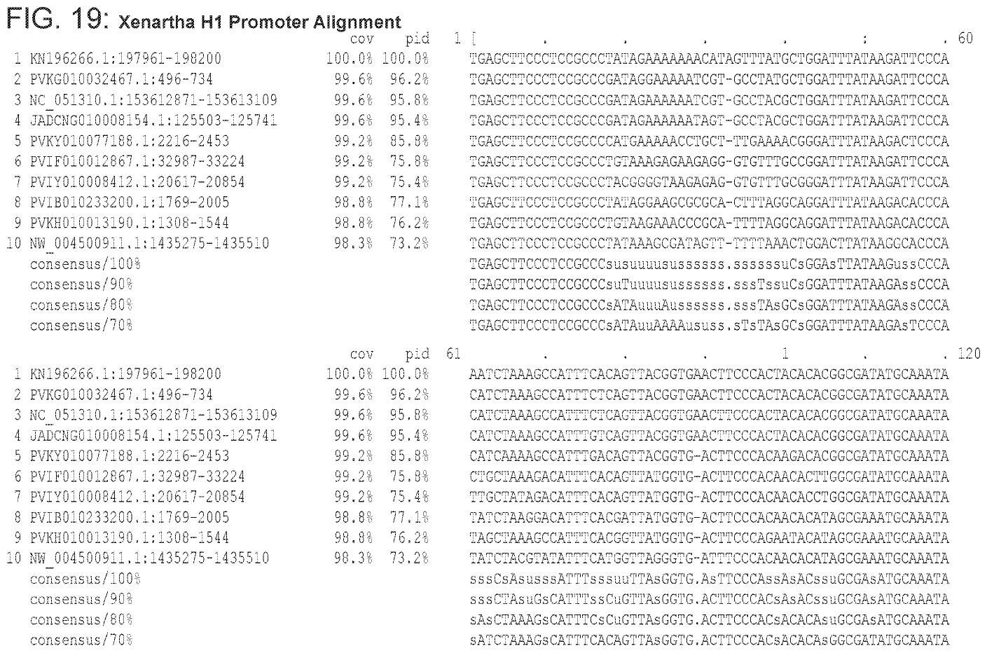

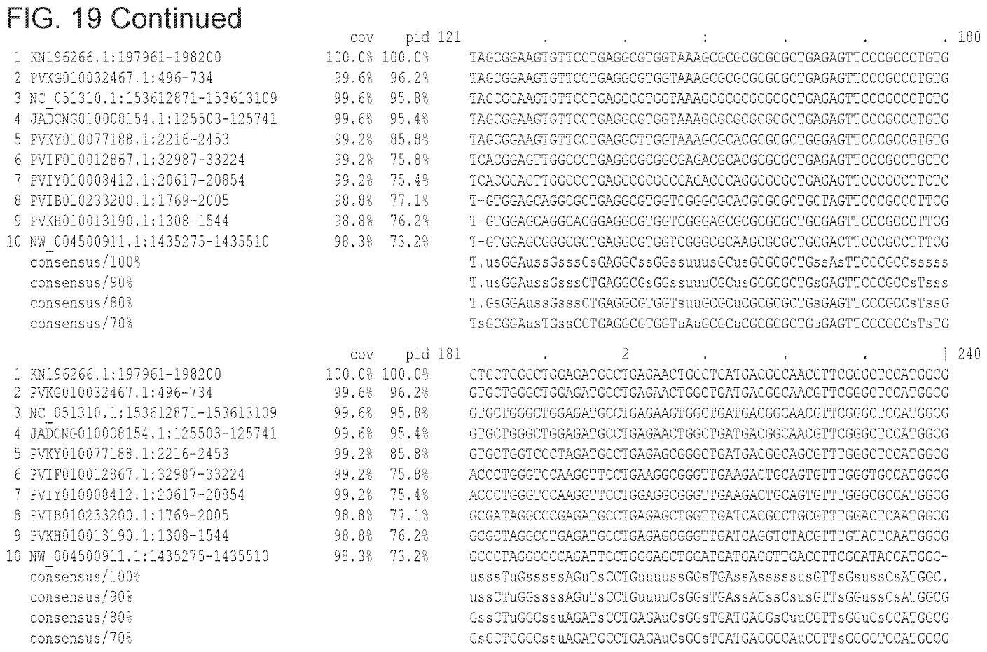

In certain embodiments, the promoter comprises an Xenarthra H1 promoter. An alignment of Xenarthra H1 promoter sequences is provided in FIG. 19 (wherein sequences numbered 1-10 in FIG. 19 correspond to SEQ ID NOs: 910-919, and consensus/100%, consensus/90%, consensus/80% and consensus/70% correspond to SEQ ID NOs: 1864-1867, respectively) In some embodiments, the promoter comprises a nucleotide sequence having at least 85%, 90%, 95%, 96%, 97%, 98%, 99%, or 100% identity to nucleotides 20-234 of any one of the sequences in FIG. 19 or a functional fragment or variant (e.g., codon optimized) thereof.

In certain embodiments, the Xenarthra H1 promoter comprises a sequence selected from those in TABLE 16.

TABLE 16

| | |
|---|---|
| Xenarthra | TGAGCTTCCCTCCGCCCKATARRRA |
| Alignment | RMVHSVDKYBTANGCDGGATTTATA |
| consensus | AGAYWCCCAYAKCTAAAGMCATTTC |
| sequence | WCRGTTAYGGTGNACTTCCCACWAC |
| 75% Identity | ACAYRGCGAWATGCAAATATNGYGG |
| | ARSWGKYSCTGAGGCGTGGTMRRGC |
| | GCRCGCGCGCTGMGAGTTCCCGCCY |
| | TKYGGYSCTRGGCYSRAGATKCCTG |
| | AGARCKGGYTGATGACGKCWRCGTT |
| | YGGRCKCCATGGCG |
| | (SEQ ID NO: 920) |
| Xenarthra | TGAGCTTCCCTCCGCCCKRTRRRRH |
| Alignment | RMVHVVDKYBTWNRCDGGATTTATA |
| consensus | AGAYWCCCAYWKCTAHRGMCATTTS |
| sequence | WCRGTTAYGGTGNACTTCCCACWAB |
| 85%_Identity | ACHYRGCGAWATGCAAATATNRYGG |
| | ARBWGKYSCTGAGGCGYGGYVRRRC |
| | GCR |
| | VGCGCGCTGMGAGTTCCCGCCYTBY |
| | SRYSCTRGGYYSNAGRTKCCTGRRR |
| | RCKGGYTGAWSACKKCWRYGTTYGG |
| | RYKCMATGGCG |
| | (SEQ ID NO: 921) |
| Xenarthra | TGAGCTTCCCTCCGCCCKRTRRRRH |
| Alignment | RMVHVVDKYBTWNRCDGGATTTATA |
| consensus | AGAYWCCCAYWKCTAHRGMCATTTS |
| sequence | WCRGTTAYGGTGNACTTCCCACWAB |
| 90%_Identity | ACHYRGCGAWATGCAAATATNRYGG |
| | ARBWGKYSCTGAGGCGYGGYVRRRC |
| | GCRVGCGCGCTGMGAGTTCCCGCCY |
| | TBYSRYSCTRGGYYSNAGRTKCCTG |
| | RRRRCKGGYTGAWSACKKCWRYGTT |
| | YGGRYKCMATGGCG |
| | (SEQ ID NO: 922) |
| Xenarthra | TGAGCTTCCCTCCGCCCBRYRRRRH |
| Alignment | RMNNVNDNBYBWWNRCNGGAYTTAT |
| consensus | AAGRYWCCCAHWKCWAHRKMYATTT |
| sequence | SWYRRTTABGGTGNAYTTCCCASWA |
| 95%_Identity | BACHYRGCGAWATGCAAATATNRYG |
| | GARBDGKYVCKGAGGCKYGGYVRRR |
| | MGCRVGCGCGCTGVKASTTCCCGCC |
| | BKBYSRYSMTRGKYYBNAGRTKCCT |
| | GRRRRSKGGHTGAWSASKBYDRYGT |
| | TYGKRYDCMATGGCG |
| | (SEQ ID NO: 923) |
| Xenarthra | TGAGCTTCCCTCCGCCCBRYRRRRH |
| Alignment | RMNNVNDNBYBWWNRCNGGAYTTAT |
| consensus | AAGRYWCCCAHWKCWAHRKMYATTT |
| sequence | SWYRRTTABGGTGNAYTTCCCASWA |
| 99% Identity | BACHYRGCGAWATGCAAATATNRYG |
| | GARBDGKYVCKGAGGCKYGGYVRRR |
| | MGCRVGCGCGCTGVKASTTCCCGCC |
| | BKBYSRYSMTRGKYYBNAGRTKCCT |
| | GRRRRSKGGHTGAWSASKBYDRYGT |
| | TYGKRYDCMATGGCG |
| | (SEQ ID NO: 924) |
| Xenarthra | TGAGCTTCCCTCCGCCCBRYRRRRH |
| Alignment | RMNNVNDNBYBWWNRCNGGAYTTAT |
| consensus | AAGRYWCCCAHWKCWAHRKMYATTT |
| sequence | SWYRRTTABGGTGNAYTTCCCASWA |
| 100%_Identity | BACHYRGCGAWATGCAAATATNRYG |
| | GARBDGKYVCKGAGGCKYGGYVRRR |
| | MGCRVGCGCGCTGVKASTTCCCGCC |
| | BKBYSRYSMTRGKYYBNAGRTKCCT |
| | GRRRRSKGGHTGAWSASKBYDRYGT |
| | TYGKRYDCMATGGCG |
| | (SEQ ID NO: 925) |

In some embodiments, the promoter comprises a nucleotide sequence having at least 85%, 90%, 95%, 96%, 97%, 98%, 99%, or 100% identity to nucleotides 20-233 of any one of SEQ ID NOs: 920-925 or a functional fragment or variant (e.g., codon optimized) thereof.

Gar1 promoters

A custom perl script was developed to compare the 5′ transcriptional start sites of pol III genes with that of pol II genes. The results were filtered for those that are orientated in opposite directions (divergent transcription). One compact bidirectional promoter identified using this method was the Gar1 promoter. On one side, the GAR1 promoter expresses the GAR1 protein, which is involved with snoRNAs, rRNA processing, and telomerase activity. The GAR1 protein appears to be expressed in all tissues, suggesting that the GAR1 promoter can drive expression ubiquitously (https://www.proteinatlas.org/ENSG00000109534-GAR1/tissue). On the other side, it expresses a lncRNA (AC126283.1 or ENSG00000272795) with unknown function, and high expression in the testis.

Accordingly in certain embodiments, the promoter is a Gar1 promoter. In certain embodiments, the Gar1 promoter is a mammalian promoter, e.g., a human Gar1 promoter, a carnivora Gar1 promoter, a primate Gar1 promoter, or a rodent Gar1 promoter. In some embodiments, the Gar1 promoter comprises a nucleotide sequence having at least 85%, 90%, 95%, 96%, 97%, 98%, 99%, or 100% identity of any one of SEQ ID NOs: 107-203 or a codon-optimized variant and/or fragment thereof. In some embodiments, the promoter comprises the nucleotide sequence of any one of SEQ ID NOs: 107-203 or a codon-optimized variant and/or fragment thereof.

In certain embodiments, a functional fragment comprises a truncation of from about 10 bases to about 70 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 10 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 20 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 30 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 40 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 50 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 60 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203). In certain embodiments, a functional fragment comprises a truncation of about 70 bases at the 5′ end, the 3′ end, or both the 5′ and 3′ ends of any one of SEQ ID NOs: 107-203 or a variant thereof (e.g., a variant having at least 85%, 90%, 95%, 96%, 97%, 98%, 99%, or 100% identity to any one of SEQ ID NOs: 107-203).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83.

In certain embodiments, the Gar1 promoter comprises a TATA mutation. In certain embodiments, the TATA mutation is a TATAA→TCGAA mutation.

In certain embodiments, a nucleic acid comprising a Gar1 promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a Gar1 promoter described herein further comprises a terminator sequence. In certain embodiments, the terminator sequence comprises one of the terminator sequences in TABLE 17.

TABLE 17

| | |
|---|---|
| a synthetic | AATAAAATATCTTTATTTTCATTAC |
| poly(A) | ATCTGTGTGTTGGTTTTTTGTGTG |
| sequence (SPA) | (SEQ ID NO: 258) |
| SPA and Pause | AATAAAATATCTTTATTTTCATTAC |
| | ATCTGTGTGTTGGTTTTTTGTGTGA |
| | ATCGATAGTACTAACATACGCTCTC |
| | CATCAAAACAAAACGAAACAAAACA |
| | AACTAGCAAAATAGGCTGTCCCCAG |
| | TGCAAGTGCAGGTGCCAGAACATTT |
| | CTCT |
| | (SEQ ID NO: 259); |
| SV40 (240 bp) | ATCTAGATAACTGATCATAATCAGC |
| | CATACCACATTTGTAGAGGTTTTAC |
| | TTGCTTTAAAAAACCTCCCACACCT |
| | CCCCCTGAACCTGAAACATAAAATG |
| | AATGCAATTGTTGTTGTTAACTTGT |
| | TTATTGCAGCTTATAATGGTTACAA |
| | ATAAAGCAATAGCATCACAAATTTC |
| | ACAAATAAAGCATTTTTTTCACTGC |
| | ATTCTAGTTGTGGTTTGTCCAAACT |
| | CATCAATGTATCTTA |
| | (SEQ ID NO: 260) |
| SV 40-mini | TTGTTTATTGCAGCTTATAATGGTT |
| (120 bp) | ACAAATAAAGCAATAGCATCACAAA |
| | TTTCACAAATAAAGCATTTTTTTCA |
| | CTGCATTCTAGTTGTGGTTTGTCCA |
| | AACTCATCAATGTATCTTAT |
| | (SEQ ID NO: 261) |
| bGH poly A | CGACTGTGCCTTCTAGTTGCCAGCC |
| | ATCTGTTGTTTGCCCCTCCCCCGTG |
| | CCTTCCTTGACCCTGGAAGGTGCCA |
| | CTCCCACTGTCCTTTCCTAATAAAA |
| | TGAGGAAATTGCATCGCATTGTCTG |
| | AGTAGGTGTCATTCTATTCTGGGGG |
| | GTGGGGTGGGGCAGGACAGCAAGGG |
| | GGAGGATTGGGAAGACAATAGCAGG |
| | CATGCTGGGGATGCGGTGGGCTCTA |
| | TGG |
| | (SEQ ID NO: 262) |
| TKpoly A | GGGGGAGGCTAACTGAAACACGGAA |
| | GGAGACAATACCGGAAGGAACCCGC |
| | GCTATGACGGCAATAAAAAGACAGA |
| | ATAAAACGCACGGGTGTTGGGTCGT |
| | TTGTTCATAAACGCGGGGTTCGGTC |
| | CCAGGGCTGGCACTCTGTCGATACC |
| | CCACCGAGACCCCATTGGGGCCAAT |
| | ACGCCCGCGTTTCTTCCTTTTCCCC |
| | ACCCCACCCCCCAAGTTCGGGTGAA |
| | GGCCCAGGGCTCGCAGCCAACGTCG |
| | GGGCGGCAGGCCCTGCCATAG |
| | (SEQ ID NO: 263) |
| SNRPl | GGTATCAAATAAAATACGAAATGTG |
| | ACAGATT |
| | (SEQ ID NO: 264) |
| SNRPla | AAATAAAATACGAAATGTGACAGAT |
| | T |
| | (SEQ ID NO: 265) |
| Histone H4B | GGTTGCTGATTTCTCCACAGCTTGC |
| | ATTTCTGAACCAAAGGCCCTTTTCA |
| | GGGCCGCCCAACTAAACAAAAGAAG |
| | AGCTGTATCCATTAAGTCAAGAAGC |
| | (SEQ ID NO: 266) |
| MALAT-1 | GATTCGTCAGTAGGGTTGTAAAGGT |
| | TTTTCTTTTCCTGAGAAAACAACCT |
| | TTTGTTTTCTCAGGTTTTGCTTTTT |
| | GGCCTTTCCCTAGCTTTAAAAAAAA |
| | AAAAGCAAAAGACGCTGGTGGCTGG |
| | CACTCCTGGTTTCCAGGACGGGGTT |
| | CAAGTCCCTGCGGTGTCTTTGCTT |
| | (SEQ ID NO: 267) |
| MALAT-comp14 | AAAGGTTTTTCTTTTCCTGAGAAAT |
| | TTCTCAGGTTTTGCTTTTTAAAAAA |
| | AAAGCAAAAGACGCTGGTGGCTGGC |
| | ACTCCTGGTTTCCAGGACGGGGTTC |
| | AAGTCCCTGCGGTGTCTTTGCTT |
| | (SEQ ID NO: 268) |

In certain embodiments, the Gar1 promoter is coupled with a viral intron (e.g., an SV40i intron, a MVM intron, a Mv2 intron, an HNRNPH1 intron, chimeric introns or synthetic introns).

In certain embodiments, the Gar1 promoter does not comprise a viral promoter and/or a synthetic promoter.

In certain embodiments, the Gar1 promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a Gar1 promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### Other Bidirectional Promoters

Using the custom perl script described above, additional bidirectional promoters were identified that can be used according to the methods described herein. In certain embodiments, the promoter is a bidirectional promoter comprising a nucleotide sequence having at least 85%, 90%, 95%, 96%, 97%, 98%, 99%, or 100% identity of any one of SEQ ID NOs: 204-255 or a codon-optimized variant and/or fragment thereof. In some embodiments, the bidirectional promoter comprises the nucleotide sequence of any one of SEQ ID NOs: 204-255 or a codon-optimized variant and/or fragment thereof.

In certain embodiments, a functional fragment comprises a truncation of from about 10 bases to about 70 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 10 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 20 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 30 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 40 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 50 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 60 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255). In certain embodiments, a functional fragment comprises a truncation of about 70 bases at the 5′ end, the 3′ end, or both the 5′ and 3′ ends of any one of SEQ ID NOs: 204-255 or a variant thereof (e.g., a variant having at least 85%, 90%, 95%, 96%, 97%, 98%, 99%, or 100% identity to any one of SEQ ID NOs: 204-255).

In certain embodiments, the functional fragment comprise at least a transcription factor binding site. Identification of transcription factor binding sites can be determined by consensus, or by using a differential distance matrix or multidimensional scaling (De Bleser P. et al. (2007) Genome Biol 8(5):R83.

In certain embodiments, the promoter comprises a TATA mutation. In certain embodiments, the TATA mutation is a TATAA→TCGAA mutation.

In certain embodiments, the promoter is not one or more of an SRP-RPS29 promoter (SEQ ID NO: 241), a 7sk1 promoter (SEQ ID NO: 242), a 7sk2 promoter (SEQ ID NO: 243), a 7sk3 promoter (SEQ ID NO: 244), an RMRP-CCDC107 promoter (SEQ ID NO: 245), an ALOXE3 promoter (SEQ ID NO: 246), a CGB1 promoter (SEQ ID NO: 247), a CGB2 promoter (SEQ ID NO: 248), a Med16-1 promoter (SEQ ID NO: 249), a Med16-2 promoter (SEQ ID NO: 250), a DPP9-1 promoter (SEQ ID NO: 251), a DPP9-2 promoter (SEQ ID NO: 252), a DPP9-3 promoter (SEQ ID NO: 253), a SNORD13-C8orf41 promoter (SEQ ID NO: 254), and a THEM259 promoter (SEQ ID NO: 255).

In certain embodiments, a nucleic acid comprising a bidirectional promoter described herein further comprises a 5′UTR including at least a portion of a beta-globin 5′UTR sequence or a Kozak sequence. In certain embodiments, the 5′UTR includes the nucleotide sequence 5′-GCCGCCACC-3′ (SEQ ID NO: 256), or a 6 bp, a 7 bp, or an 8 bp fragment thereof. In certain embodiments, the 6 bp fragment is 5′-GCCACC-3′ (SEQ ID NO: 257).

In certain embodiments, a nucleic acid comprising a bidirectional promoter described herein further comprises a terminator sequence. In certain embodiments, the terminator sequence comprises one of the terminator sequences in TABLE 18.

TABLE 18

| | |
|---|---|
| a synthetic | AATAAAATATCTTTATTTTCATTAC |
| poly(A) | ATCTGTGTGTTGGTTTTTTGTGTG |
| sequence (SPA) | (SEQ ID NO: 258) |
| SPA and Pause | AATAAAATATCTTTATTTTCATTAC |
| | ATCTGTGTGTTGGTTTTTTGTGTGA |
| | ATCGATAGTACTAACATACGCTCTC |
| | CATCAAAACAAAACGAAACAAAACA |
| | AACTAGCAAAATAGGCTGTCCCCAG |
| | TGCAAGTGCAGGTGCCAGAACATTT |
| | CTCT |
| | (SEQ ID NO: 259); |
| SV40 (240 bp) | ATCTAGATAACTGATCATAATCAGC |
| | CATACCACATTTGTAGAGGTTTTAC |
| | TTGCTTTAAAAAACCTCCCACACCT |
| | CCCCCTGAACCTGAAACATAAAATG |
| | AATGCAATTGTTGTTGTTAACTTGT |
| | TTATTGCAGCTTATAATGGTTACAA |
| | ATAAAGCAATAGCATCACAAATTTC |
| | ACAAATAAAGCATTTTTTTCACTGC |
| | ATTCTAGTTGTGGTTTGTCCAAACT |
| | CATCAATGTATCTTA |
| | (SEQ ID NO: 260) |
| SV 40-mini | TTGTTTATTGCAGCTTATAATGGTT |
| (120 bp) | ACAAATAAAGCAATAGCATCACAAA |
| | TTTCACAAATAAAGCATTTTTTTCA |
| | CTGCATTCTAGTTGTGGTTTGTCCA |
| | AACTCATCAATGTATCTTAT |
| | (SEQ ID NO: 261) |
| bGH poly A | CGACTGTGCCTTCTAGTTGCCAGCC |
| | ATCTGTTGTTTGCCCCTCCCCCGTG |
| | CCTTCCTTGACCCTGGAAGGTGCCA |
| | CTCCCACTGTCCTTTCCTAATAAAA |
| | TGAGGAAATTGCATCGCATTGTCTG |
| | AGTAGGTGTCATTCTATTCTGGGGG |
| | GTGGGGTGGGGCAGGACAGCAAGGG |
| | GGAGGATTGGGAAGACAATAGCAGG |
| | CATGCTGGGGATGCGGTGGGCTCTA |
| | TGG |
| | (SEQ ID NO: 262) |
| TKpoly A | GGGGGAGGCTAACTGAAACACGGAA |
| | GGAGACAATACCGGAAGGAACCCGC |
| | GCTATGACGGCAATAAAAAGACAGA |
| | ATAAAACGCACGGGTGTTGGGTCGT |
| | TTGTTCATAAACGCGGGGTTCGGTC |
| | CCAGGGCTGGCACTCTGTCGATACC |
| | CCACCGAGACCCCATTGGGGCCAAT |
| | ACGCCCGCGTTTCTTCCTTTTCCCC |
| | ACCCCACCCCCCAAGTTCGGGTGAA |
| | GGCCCAGGGCTCGCAGCCAACGTCG |
| | GGGCGGCAGGCCCTGCCATAG |
| | (SEQ ID NO: 263) |
| SNRPl | GGTATCAAATAAAATACGAAATGTG |
| | ACAGATT |
| | (SEQ ID NO: 264) |
| SNRPla | AAATAAAATACGAAATGTGACAGAT |
| | T |
| | (SEQ ID NO: 265) |
| Histone H4B | GGTTGCTGATTTCTCCACAGCTTGC |
| | ATTTCTGAACCAAAGGCCCTTTTCA |
| | GGGCCGCCCAACTAAACAAAAGAAG |
| | AGCTGTATCCATTAAGTCAAGAAGC |
| | (SEQ ID NO: 266) |
| MALAT-1 | GATTCGTCAGTAGGGTTGTAAAGGT |
| | TTTTCTTTTCCTGAGAAAACAACCT |
| | TTTGTTTTCTCAGGTTTTGCTTTTT |
| | GGCCTTTCCCTAGCTTTAAAAAAAA |
| | AAAAGCAAAAGACGCTGGTGGCTGG |
| | CACTCCTGGTTTCCAGGACGGGGTT |
| | CAAGTCCCTGCGGTGTCTTTGCTT |
| | (SEQ ID NO: 267) |
| MALAT-comp14 | AAAGGTTTTTCTTTTCCTGAGAAAT |
| | TTCTCAGGTTTTGCTTTTTAAAAAA |
| | AAAGCAAAAGACGCTGGTGGCTGGC |
| | ACTCCTGGTTTCCAGGACGGGGTTC |
| | AAGTCCCTGCGGTGTCTTTGCTT |
| | (SEQ ID NO: 268) |

In certain embodiments, the bidirectional promoter is coupled with a viral intron (e.g., an SV40i intron, a MVM intron, a Mv2 intron, an HNRNPH1 intron, chimeric introns or synthetic introns).

In certain embodiments, the bidirectional promoter does not comprise a viral promoter and/or a synthetic promoter. In certain embodiments, the compact promoter does not comprise F5tg83.

In certain embodiments, the bidirectional promoter comprises at least 95%, 98%, 99%, 99.5% or 100% identity to a naturally-occurring mammalian promoter. In certain embodiments, the compact promoter comprises at least 95%, 98%, 99%, 99.5% or 100% identity to a naturally-occurring human promoter.

The expression level of a bidirectional promoter can be determined by expressing a reporter molecule in a cell, e.g., a human embryonic kidney (HEK) cell line or an N2A cell line. In certain embodiments, the compact promoter is capable of expressing a luciferase reporter at a higher level than is a HSK thymidine kinase (TK) promoter.

### III. Nuclease Systems

In general, a “nuclease system” refers collectively to transcripts and other elements involved in the expression of or directing the activity of a gene encoding a gene-editing nuclease (e.g., a Cas nuclease) and a guide sequence (also referred to as a “spacer” in the context of certain endogenous gene editing systems, e.g., a CRISPR system).

In general, “CRISPR system” refers collectively to transcripts and other elements involved in the expression of or directing the activity of CRISPR-associated (“Cas”) genes, including sequences encoding a Cas gene, a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

As used herein, “target sequence” refers to a sequence to which a guide sequence is designed to have complementarity, where hybridization between a target sequence and a guide sequence promotes the formation of a gene editing nuclease complex (e.g., a CRISPR complex). Full complementarity is not necessarily required, provided there is sufficient complementarity to cause hybridization and promote formation of a gene editing nuclease complex (e.g., a CRISPR complex). A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the presently disclosed subject matter, an exogenous template polynucleotide may be referred to as an editing template. In an aspect of the presently disclosed subject matter the recombination is homologous recombination.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. When multiple different guide sequences are used, a single expression construct may be used to target nuclease activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, more such guide-sequence-containing vectors may be provided, and optionally delivered to a cell.

In some embodiments, a vector comprises a regulatory element operably linked to an enzyme-coding sequence encoding a nuclease, such as a CRISPR enzyme (e.g., a Cas protein). Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known: for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae.

In some embodiments, the nuclease can be any endonuclease that is capable of cleaving DNA to effect a single or double strand break at the intended locus. For example, the nuclease can be a MAD1, MAD2, MAD3, MAD4, MAD5, MAD6, MAD7, MAD8, MAD9 MAD10, MAD11, or MAD11 endonuclease (see, e.g., U.S. Pat. No. 9,982,279). The DNA endonuclease can be a Cpf1 endonuclease: a homolog thereof, a recombinant of the naturally occurring molecule thereof, a codon-optimized version thereof, a modified version thereof (e.g., a mutated variant such as a nickase), and combinations of any of the foregoing. For example, in some embodiments, the DNA endonuclease is a Cas9 or Cpf1 endonuclease that effects a single-strand break (SSB) or double-strand break (DSB) at a locus within or near a target sequence.

In some embodiments, the DNA endonuclease is a Cas9 endonuclease (e.g., a recombinant Cas9, a codon-optimized Cas9, a modified or mutated Cas9). The Cas9 endonuclease can be derived from a variety of bacterial species. For example, in certain embodiments, the Cas9 endonuclease is derived from Streptococcus thermophiles, Streptococcus pyogenes. Neisseria meningitides. Staphylococcus aureus, or Treponema denticola. In a specific embodiment, the Cas9 endonuclease is derived from Staphylococcus aureus (SaCas9). In another specific embodiment, the Cas9) endonuclease is derived from Streptococcus pyogenes (SpCas9). Wild type Cas9 has two active sites (RuvC and HNH nuclease domains) for cleaving DNA, one for each strand of the double helix. However, nickase variants of Cas9 are readily available (e.g., Addgene, plasmid #: 48873) that are only capable of cleaving one strand of the DNA due to catalytic inactivation of the RuvC or HNH nuclease domains. Accordingly, in a specific embodiment, the Cas9 endonuclease is a mutated SpCas9 endonuclease (e.g., a nickase) and/or a codon-optimized version thereof.

In other embodiments, the DNA endonuclease is a Cpf1 endonuclease (e.g., a recombinant Cpf1, a codon-optimized Cpf1, a modified or mutated Cpf1). The Cpf1 endonuclease can be derived from a variety of bacterial species. For example, in certain embodiments, the Cpf1 endonuclease is derived from Acidaminococcus bacteria or Lachnospiraceae bacteria. In a specific embodiment, the Cpf1 endonuclease is a Lachnospiraceae bacterium ND2006 Cpf1.

In other embodiments, the DNA endonuclease is a MAD7 endonuclease (e.g., a recombinant MAD7, a codon-optimized MAD7, a modified or mutated MAD7). MAD7 is a codon optimized endonuclease can be derived from Eubacterium rectale (Inscripta, Boulder, CO.) MAD7 is described in U.S. Pat. No. 9,982,279.

In other embodiments, an RNA-guided nuclease is used. Exemplary RNA-guided nucleases include Cas13a, Cas13b and Cas13d.

In some embodiments, the nuclease (e.g., a CRISPR) directs cleavage of one or both strands at the location of a target sequence, such as within the target sequence and/or within the complement of the target sequence. In some embodiments, the nuclease directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a nuclease that is mutated to with respect to a corresponding wild-type enzyme such that the mutated nuclease lacks the ability to cleave one or both strands of a target polynucleotide containing a target sequence. For example, in certain embodiments, a nuclease system comprises a nuclease-dead version of a nuclease (e.g., Cas9 (dCas9)) (Qi et al. (2013) CELL 152, 1173-1183; Gilbert et al. (2013) CELL 154, 442-451: Larson et al. (2013) NATURE PROTOCOLS 8, 2180-2196: Fuller et al. (2014) ADVANCES IN EXPERIMENTAL MEDICINE AND BIOLOGY 801, 773-781). Instead of inducing cleavage, a nuclease-dead nuclease stays bound tightly to a target sequence. When targeted to an actively transcribed gene, inhibition of pol II progression through a steric hindrance mechanism can lead to efficient transcriptional repression. Thus, use of a nuclease-dead nuclease can achieve therapeutic repression of a target gene without inducing a break in the target nucleotide sequence.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. See Nakamura et al. (2000) NUCL. ACIDS RES. 28:292. Computer algorithms for codon optimizing a particular sequence for expression in a particular host cell are also available, such as Gene Forge (Aptagen: Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length.

The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In an aspect of the presently disclosed subject matter, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the presently disclosed subject matter, the DNA molecule encoding the gene product may be introduced into the cell via a vector. In a preferred embodiment of the presently disclosed subject matter the gene product is luciferase. In a further embodiment of the presently disclosed subject matter the expression of the gene product is decreased.

### IV. Vector Systems

Several aspects of the presently disclosed subject matter relate to vector systems comprising one or more vectors, or vectors as such. Vectors can be designed for expression of CRISPR transcripts (e.g. nucleic acid transcripts, proteins, or enzymes) in prokaryotic or eukaryotic cells. For example, CRISPR transcripts can be expressed in bacterial cells such as Escherichia coli, insect cells (using baculovirus expression vectors), yeast cells, or mammalian cells. Suitable host cells are discussed further in Goeddel (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Vectors may be introduced and propagated in a prokaryote. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion vectors add a number of amino acids to a protein encoded therein, such as to the amino terminus of the recombinant protein. Such fusion vectors may serve one or more purposes, such as: (i) to increase expression of recombinant protein: (ii) to increase the solubility of the recombinant protein: and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc: Smith and Johnson (1988) GENE 67:31-40), pMAL (New England Biolabs, Beverly, Mass.) and pRIT5 (Pharmacia, Piscataway N.J.) that fuse glutathione S-transferase (GST), maltose E binding protein, or protein A, respectively, to the target recombinant protein.

Examples of suitable inducible non-fusion E. coli expression vectors include pTrc (Amrann et al. (1988) GENE 69:301-315) and pET 11d (Studier et al. (1990) Gene Expression Technology: Methods in Enzymology 185, Academic Press, San Diego, Calif.).

In some embodiments, a vector is a yeast expression vector. Examples of vectors for expression in yeast Saccharomyces cerevisiae include pYepSec1 (Baldari, et al. (1987) EMBO J. 6:229-234), pMFa (Kuijan and Herskowitz (1982) CELL 30: 933-943), pJRY88 (Schultz et al. (1987) GENE 54:113-123), pYES2 (Invitrogen Corporation, San Diego, Calif.), and picZ (InVitrogen Corp, San Diego, Calif.).

In some embodiments, a vector is capable of driving expression of one or more sequences in mammalian cells using a mammalian expression vector. Examples of mammalian expression vectors include pCDM8 (Seed (1987) NATURE 329:840) and pMT2PC (Kaufman et al. (1987) EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook et al. (1989) Molecular Cloning: A Laboratory Manual. 2nd ed., Cold Spring Harbor Laboratory, Cold Spring Harbor Laboratory Press, Cold Spring Harbor, N.Y.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific: Pinkert et al. (1987) GENES DEV. 1:268-277), lymphoid-specific promoters (Calame and Eaton (1988) ADV. IMMUNOL. 43:235-275), in particular promoters of T cell receptors (Winoto and Baltimore (1989) EMBO J. 8:729-733) and immunoglobulins (Baneiji et al. (1983) CELL 33:729-740: Queen and Baltimore (1983) CELL 33:741-748) neuron-specific promoters (e.g., the neurofilament promoter: Byrne and Ruddle (1989) PROC. NATL. ACAD. SCI. USA 86:5473-5477), pancreas-specific promoters (Edlund et al. (1985) SCIENCE 230:912-916), and mammary gland-specific promoters (e.g., milk whey promoter: U.S. Pat. No. 4,873,316 and European Application Publication. No. 264,166). Developmentally-regulated promoters are also encompassed, e.g., the murine hox promoters (Kessel and Gruss (1990) SCIENCE 249: 374-379) and the .alpha.-fetoprotein promoter (Campes and Tilghman (1989) GENES DEV. 3:537-546).

In some embodiments, a regulatory element is operably linked to one or more elements of a CRISPR system so as to drive expression of the one or more elements of the CRISPR system. In general, CRISPRs (Clustered Regularly Interspaced Short Palindromic Repeats), also known as SPIDRs (SPacer Interspersed Direct Repeats), constitute a family of DNA loci that are usually specific to a particular bacterial species. The CRISPR locus comprises a distinct class of interspersed short sequence repeats (SSRs) that were recognized in E. coli (Ishino et al. (1987) J. BACTERIOL., 169:5429-5433; and Nakata et al. (1989) J. BACTERIOL., 171:3553-3556), and associated genes. Similar interspersed SSRs have been identified in Haloferax mediterranei, Streptococcus pyogenes, Anabaena, and Mycobacterium tuberculosis (Groenen et al. (1993) MOL. MICROBIOL., 10:1057-1065; Hoe et al. (1999) EMERG. INFECT. DIS., 5:254-263: Masepohl et al. (1996) BIOCHIM. BIOPHYS. ACTA 1307:26-30; and Mojica et al. (1995) MOL. MICROBIOL., 17:85-93). The CRISPR loci typically differ from other SSRs by the structure of the repeats, which have been termed short regularly spaced repeats (SRSRs) (Janssen et al. (2002) OMICS J. INTEG. BIOL., 6:23-33; and Mojica et al. (2000) MOL. MICROBIOL., 36:244-246). In general, the repeats are short elements that occur in clusters that are regularly spaced by unique intervening sequences with a substantially constant length (Mojica et al. (2000) MOL. MICROBIOL., 36:244-246). Although the repeat sequences are highly conserved between strains, the number of interspersed repeats and the sequences of the spacer regions typically differ from strain to strain (van Embden et al. (2000) J. BACTERIOL., 182:2393-2401). CRISPR loci have been identified in more than 40 prokaryotes (e.g., Jansen et al. (2002) MOL. MICROBIOL., 43:1565-1575: and Mojica et al. (2005) J. Mol. Evol. 60:174-82) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thernioplasnia, Corynebacterium, Mycobacterium, Streptomyces, Aquifrx, Porphyromonas, Chlorobium. Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myrococcus, Campylobacter, Wolinella, Acinetobacter, Erwinia, Escherichia, Legionella, Methylococcus, Pasteurella, Photobacterium, Salmonella, Xanthomonuas, Yersinia, Treponema, and Thermotoga.

### V. Construction of rAAV Vectors

The disclosure provides recombinant AAV (rAAV) vectors comprising a nuclease system under the control of a suitable promoter (e.g., a compact bidirectional promoter) to direct the expression of the gRNA and nuclease. The disclosure further provides a therapeutic composition comprising an rAAV vector comprising a nuclease system under the control of a suitable promoter (e.g., a compact bidirectional promoter). A variety of rAAV vectors may be used to deliver the desired complement system gene to the appropriate cells and/or tissues and to direct its expression. More than 30 naturally occurring serotypes of AAV from humans and non-human primates are known. Many natural variants of the AAV capsid exist, and an rAAV vector of the disclosure may be designed based on an AAV with properties specifically suited for expression in the cells and/or tissues relevant for the nuclease system to be expressed.

In general, an rAAV vector is comprised of, in order, a 5′ adeno-associated virus inverted terminal repeat, a transgene or gene of interest encoding a nuclease system operably linked to a sequence which regulates its expression in a target cell, and a 3′ adeno-associated virus inverted terminal repeat. In addition, the rAAV vector may preferably have a polyadenylation sequence. Generally, rAAV vectors should have one copy of the AAV ITR at each end of the transgene or gene of interest, in order to allow replication, packaging, and efficient integration into cell chromosomes. Within preferred embodiments of the disclosure, the transgene sequence encoding a complement system polypeptide (or a functional fragment or variant thereof) or a biologically active fragment thereof will be of about 2 to 5 kb in length (or alternatively, the transgene may additionally contain a “stuffer” or “filler” sequence to bring the total size of the nucleic acid sequence between the two ITRs to between 2 and 5 kb).

Recombinant AAV vectors of the present disclosure may be generated from a variety of adeno-associated viruses. For example, ITRs from any AAV serotype are expected to have similar structures and functions with regard to replication, integration, excision and transcriptional mechanisms. Examples of AAV serotypes include AAV1, AAV2, AAV3, AAV4, AAV5, AAV6, AAV7, AAV8, AAV9, AAV10, AAV11 and AAV12. In some embodiments, the rAAV vector is generated from serotype AAV1, AAV2, AAV4, AAV5, or AAV8. These serotypes are known to target photoreceptor cells or the retinal pigment epithelium. In particular embodiments, the rAAV vector is generated from serotype AAV2. In certain embodiments, the AAV serotypes include AAVrh8, AAVrh8R or AAVrh10. It will also be understood that the rAAV vectors may be chimeras of two or more serotypes selected from serotypes AAV 1 through AAV12. The tropism of the vector may be altered by packaging the recombinant genome of one serotype into capsids derived from another AAV serotype. In some embodiments, the ITRs of the rAAV virus may be based on the ITRs of any one of AAV 1-12 and may be combined with an AAV capsid selected from any one of AAV1-12, AAV-DJ, AAV-DJ8, AAV-DJ9 or other modified serotypes. In certain embodiments, any AAV capsid serotype may be used with the vectors of the disclosure.

Examples of AAV serotypes include AAV1, AAV2, AAV3, AAV4, AAV5, AAV6, AAV7, AAV8, AAV9, AAV10, AAV11, AAV 12, AAV-DJ, AAV-DJ8, AAV-DJ9, AAVrh8, AAVrh8R or AAVrh10. In certain embodiments, the AAV capsid serotype is AAV2.

Desirable AAV fragments for assembly into vectors may include the cap proteins, including the vp 1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAV capsid, a recombinant AAV capsid, or a “humanized” AAV capsid.

Pseudotyped vectors, wherein the capsid of one AAV is replaced with a heterologous capsid protein, are useful in the disclosure. In some embodiments, the AAV is AAV2/5. In another embodiment, the AAV is AAV2/8. When pseudotyping an AAV vector, the sequences encoding each of the essential rep proteins may be supplied by different AAV sources (e.g., AAV1, AAV2, AAV3, AAV4, AAV5, AAV6, AAV7, AAV8). For example, the rep78/68 sequences may be from AAV2, whereas the rep52/40 sequences may be from AAV8.

In one embodiment, the vectors of the disclosure contain, at a minimum, sequences encoding a selected AAV serotype capsid, e.g., an AAV2 capsid or a fragment thereof. In another embodiment, the vectors of the disclosure contain, at a minimum, sequences encoding a selected AAV serotype rep protein, e.g., AAV2 rep protein, or a fragment thereof.

Optionally, such vectors may contain both AAV cap and rep proteins. In vectors in which both AAV rep and cap are provided, the AAV rep and AAV cap sequences can both be of one serotype origin, e.g., all AAV2 origin. In certain embodiments, the vectors may comprise rep sequences from an AAV serotype which differs from that which is providing the cap sequences. In some embodiments, the rep and cap sequences are expressed from separate sources (e.g., separate vectors, or a host cell and a vector). In some embodiments, these rep sequences are fused in frame to cap sequences of a different AAV serotype to form a chimeric AAV vector, such as AAV2/8 described in U.S. Pat. No. 7,282,199, which is incorporated by reference herein. Examples of AAV serotypes include AAV1, AAV2, AAV3, AAV4, AAV5, AAV6, AAV7, AAV8, AAV9, AAV10, AAV11, AAV 12, AAV-DJ, AAV-DJ8, AAV-DJ9, AAVrh8, AAVrh8R or AAVrh10. In some embodiments, the cap is derived from AAV2.

In some embodiments, any of the vectors disclosed herein includes a spacer, i.e., a DNA sequence interposed between the promoter and the rep gene ATG start site. In some embodiments, the spacer may be a random sequence of nucleotides, or alternatively, it may encode a gene product, such as a marker gene. In some embodiments, the spacer may contain genes which typically incorporate start/stop and polyA sites. In some embodiments, the spacer may be a non-coding DNA sequence from a prokaryote or eukaryote, a repetitive non-coding sequence, a coding sequence without transcriptional controls or a coding sequence with transcriptional controls. In some embodiments, the spacer is a phage ladder sequences or a yeast ladder sequence. In some embodiments, the spacer is of a size sufficient to reduce expression of the rep78 and rep68 gene products, leaving the rep52, rep40) and cap gene products expressed at normal levels. In some embodiments, the length of the spacer may therefore range from about 10 bp to about 10.0 kbp, preferably in the range of about 100 bp to about 8.0 kbp. In some embodiments, the spacer is less than 2 kbp in length.

In certain embodiments, the capsid is modified to improve therapy. The capsid may be modified using conventional molecular biology techniques. In certain embodiments, the capsid is modified for minimized immunogenicity, better stability and particle lifetime, efficient degradation, and/or accurate delivery of the nuclease system to the nucleus. In some embodiments, the modification or mutation is an amino acid deletion, insertion, substitution, or any combination thereof in a capsid protein. A modified polypeptide may comprise 1, 2, 3, 4, 5, up to 10, or more amino acid substitutions and/or deletions and/or insertions. A “deletion” may comprise the deletion of individual amino acids, deletion of small groups of amino acids such as 2, 3, 4 or 5 amino acids, or deletion of larger amino acid regions, such as the deletion of specific amino acid domains or other features. An “insertion” may comprise the insertion of individual amino acids, insertion of small groups of amino acids such as 2, 3, 4 or 5 amino acids, or insertion of larger amino acid regions, such as the insertion of specific amino acid domains or other features. A “substitution” comprises replacing a wild type amino acid with another (e.g., a non-wild type amino acid). In some embodiments, the another (e.g., non-wild type) or inserted amino acid is Ala (A), His (H), Lys (K), Phe (F), Met (M), Thr (T), Gin (Q), Asp (D), or Glu (E). In some embodiments, the another (e.g., non-wild type) or inserted amino acid is A. In some embodiments, the another (e.g., non-wild type) amino acid is Arg (R), Asn (N), Cys (C), Gly (G), lie (I), Leu (L), Pro (P), Ser (S), Trp (W), Tyr (Y), or Val (V). Conventional or naturally occurring amino acids are divided into the following basic groups based on common side-chain properties: (1) non-polar: Norleucine, Met, Ala, Val, Leu, He: (2) polar without charge: Cys, Ser, Thr, Asn, Gin: (3) acidic (negatively charged): Asp, Glu: (4) basic (positively charged): Lys, Arg: and (5) residues that influence chain orientation: Gly, Pro; and (6) aromatic: Trp, Tyr, Phe, His. Conventional amino acids include L or D stereochemistry. In some embodiments, the another (e.g., non-wild type) amino acid is a member of a different group (e.g., an aromatic amino acid is substituted for a non-polar amino acid). Substantial modifications in the biological properties of the polypeptide are accomplished by selecting substitutions that differ significantly in their effect on maintaining (a) the structure of the polypeptide backbone in the area of the substitution, for example, as a B-sheet or helical conformation, (b) the charge or hydrophobicity of the molecule at the target site, or (c) the bulk of the side chain. Naturally occurring residues are divided into groups based on common side-chain properties: (1) Non-polar: Norleucine, Met, Ala, Val, Leu, Ile;(2) Polar without charge: Cys, Ser, Thr, Asn, Gln;(3) Acidic (negatively charged): Asp, Glu;(4) Basic (positively charged): Lys. Arg(5) Residues that influence chain orientation: Gly, Pro: and(6) Aromatic: Trp, Tyr, Phe, His. In some embodiments, the another (e.g., non-wild type) amino acid is a member of a different group (e.g., a hydrophobic amino acid for a hydrophilic amino acid, a charged amino acid for a neutral amino acid, an acidic amino acid for a basic amino acid, etc.). In some embodiments, the another (e.g., non-wild type) amino acid is a member of the same group (e.g., another basic amino acid, another acidic amino acid, another neutral amino acid, another charged amino acid, another hydrophilic amino acid, another hydrophobic amino acid, another polar amino acid, another aromatic amino acid or another aliphatic amino acid). In some embodiments, the another (e.g., non-wild type) amino acid is an unconventional amino acid. Unconventional amino acids are non-naturally occurring amino acids. Examples of an unconventional amino acid include, but are not limited to, aminoadipic acid, beta-alanine, beta-aminopropionic acid, aminobutyric acid, piperidinic acid, aminocaprioic acid, aminoheptanoic acid, aminoisobutyric acid, aminopimelic acid, citrulline, diaminobutyric acid, desmosine, diaminopimelic acid, diaminopropionic acid, N-ethylglycine, N-ethylaspargine, hyroxylysine, allo-hydroxylysine, hydroxyproline, isodesmosine, allo-isoleucine, N-methylglycine, sarcosine, N-methylisoleucine, N-methylvaline, norvaline, norleucine, orithine, 4-hydroxyproline, Y-carboxyglutamate, ε-N,N,N-trimethyllysine, ε-N-acetyllysine, O-phosphoserine, N-acetylserine, N-formylmethionine, 3-methylhistidine, 5-hydroxy lysine, o-N-methylarginine, and other similar amino acids and amino acids (e.g., 4-hydroxyproline). In some embodiments, one or more amino acid substitutions are introduced into one or more of VP1, VP2 and VP3. In one aspect, a modified capsid protein comprises 1, 2, 3, 4, 5, 6, 7, 8, 9, 10, 11, 12, 13, 14, or 15 conservative or non-conservative substitutions relative to the wild-type polypeptide. In another aspect, the modified capsid polypeptide of the disclosure comprises modified sequences, wherein such modifications can include both conservative and non-conservative substitutions, deletions, and/or additions, and typically include peptides that share at least 60%, at least 65%, at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to the corresponding wild-type capsid protein.

In some embodiments, the recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors: a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al., 1993 J. VIROL, 70:520-532 and U.S. Pat. No. 5,478,745, among others. These publications are incorporated by reference herein.

In some embodiments, recombinant AAVs may be produced using the triple transfection method (described in detail in U.S. Pat. No. 6,001,650). Typically, the recombinant AAVs are produced by transfecting a host cell with an recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). In some embodiments, vectors suitable for use with the present disclosure may be pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

Cells may also be transfected with a vector (e.g., helper vector) which provides helper functions to the AAV. The vector providing helper functions may provide adenovirus functions, including, e.g., E1a, E1b, E2a, E40RF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

An rAAV vector of the disclosure is generated by introducing a nucleic acid sequence encoding an AAV capsid protein, or fragment thereof: a functional rep gene or a fragment thereof: a minigene composed of, at a minimum, AAV inverted terminal repeats (ITRs) and a transgene: and sufficient helper functions to permit packaging of the minigene into the AAV capsid, into a host cell. The components required for packaging an AAV minigene into an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

In some embodiments, such a stable host cell will contain the required component(s) under the control of an inducible promoter. Alternatively, the required component(s) may be under the control of a constitutive promoter. Examples of suitable inducible and constitutive promoters are provided herein, in the discussion below of regulator elements suitable for use with the transgene, i.e., a nucleic acid comprising a nuclease system. In still another alternative, a selected stable host cell may contain selected components under the control of a constitutive promoter and other selected components under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art.

The minigene, rep sequences, cap sequences, and helper functions required for producing the rAAV of the disclosure may be delivered to the packaging host cell in the form of any genetic element which transfers the sequences. The selected genetic element may be delivered by any suitable method known in the art. See, e.g., Sambrook et al., Molecular Cloning: A Laboratory Manual, Cold Spring Harbor Press, Cold Spring Harbor, NY.

Unless otherwise specified, the AAV ITRs, and other selected AAV components described herein, may be readily selected from among any AAV serotype, including, without limitation, AAV1, AAV2, AAV3, AAV4, AAV5, AAV6, AAV7, AAV8, AAV9, AAV10, AAV10, AAV11, AAV 12, AAV-DJ, AAV-DJ8, AAV-DJ9, AAVrh8, AAVrh8R or AAVrh10 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

The minigene is composed of, at a minimum, a transgene comprising a nuclease system, as described above, and its regulatory sequences, and 5′ and 3′ AAV inverted terminal repeats (ITRs). In one desirable embodiment, the ITRs of AAV serotype 2 are used. However, ITRs from other suitable serotypes may be selected. The minigene is packaged into a capsid protein and delivered to a selected host cell.

In some embodiments, regulatory sequences are operably linked to the transgene comprising a nuclease system. The regulatory sequences may include conventional regulatory elements which are operably linked to the complement system gene, splice variant, or a fragment thereof in a manner which permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences: efficient RNA processing signals such as splicing and polyadenylation (poly A) signals: sequences that stabilize cytoplasmic mRNA: sequences that enhance translation efficiency (i.e., Kozak consensus sequence): sequences that enhance protein stability: and when desired, sequences that enhance secretion of the encoded product. Numerous expression control sequences, including promoters, are known in the art and may be utilized.

The regulatory sequences useful in the constructs of the present disclosure may also contain an intron, desirably located between the promoter/enhancer sequence and the gene. In some embodiments, the intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 PROC. NATL. ACAD. SCI., USA, 96:3906-3910). Poly A signals may be derived from many suitable species, including, without limitation SV-40, human and bovine.

Another regulatory component of the rAAV useful in the method of the disclosure is an internal ribosome entry site (IRES). An IRES sequence, or other suitable systems, may be used to produce more than one polypeptide from a single gene transcript (for example, to produce more than one complement system polypeptides). An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In some embodiments, expression of the transgene comprising a nuclease system is driven by a separate promoter (e.g., a viral promoter). In certain embodiments, any promoters suitable for use in AAV vectors may be used with the vectors of the disclosure. The selection of the transgene promoter to be employed in the rAAV may be made from among a wide number of constitutive or inducible promoters that can express the selected transgene in the desired cell. Examples of suitable promoters are described in detail below.

Other regulatory sequences useful in the disclosure include enhancer sequences. Enhancer sequences useful in the disclosure include the 1RBP enhancer, immediate early cytomegalovirus enhancer, one derived from an immunoglobulin gene or SV40 enhancer, the cis-acting element identified in the mouse proximal promoter, etc.

Selection of these and other common vector and regulatory elements are well-known and many such sequences are available. See, e.g., Sambrook et al., and references cited therein at, for example, pages 3.18-3.26 and 16, 17-16.27 and Ausubel et al., Current Protocols in Molecular Biology, John Wiley & Sons, New York, 1989).

The rAAV vector may also contain additional sequences, for example from an adenovirus, which assist in effecting a desired function for the vector. Such sequences include, for example, those which assist in packaging the rAAV vector in adenovirus-associated virus particles.

The rAAV vector may also contain a reporter sequence for co-expression, such as but not limited to lacZ, GFP, CFP, YFP, RFP, mCherry, tdTomato, etc. In some embodiments, the rAAV vector may comprise a selectable marker. In some embodiments, the selectable marker is an antibiotic-resistance gene. In some embodiments, the antibiotic-resistance gene is an ampicillin-resistance gene. In some embodiments, the ampicillin-resistance gene is beta-lactamase.

In some embodiments, the rAAV particle is an ssAAV. In some embodiments, the rAAV particle is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to −55 kd) and any currently available RNA-based therapy.

The single-stranded nature of the AAV genome may impact the expression of rAAV vectors more than any other biological feature. Rather than rely on potentially variable cellular mechanisms to provide a complementary-strand for rAAV vectors, it has now been found that this problem may be circumvented by packaging both strands as a single DNA molecule. In the studies described herein, an increased efficiency of transduction from duplexed vectors over conventional rAAV was observed in He La cells (5-140 fold). More importantly, unlike conventional single-stranded AAV vectors, inhibitors of DNA replication did not affect transduction from the duplexed vectors of the invention. In addition, the inventive duplexed parvovirus vectors displayed a more rapid onset and a higher level of transgene expression than did rAAV vectors in mouse hepatocytes in vivo. All of these biological attributes support the generation and characterization of a new class of parvovirus vectors (delivering duplex DNA) that significantly contribute to the ongoing development of parvovirus-based gene delivery systems.

Overall, a novel type of parvovirus vector that carries a duplexed genome, which results in co-packaging strands of plus and minus polarity tethered together in a single molecule, has been constructed and characterized by the investigations described herein. Accordingly, the present invention provides a parvovirus particle comprising a parvovirus capsid (e.g., an AAV capsid) and a vector genome encoding a heterologous nucleotide sequence, where the vector genome is self-complementary, i.e., the vector genome is a dimeric inverted repeat. The vector genome is preferably approximately the size of the wild-type parvovirus genome (e.g., the AAV genome) corresponding to the parvovirus capsid into which it will be packaged and comprises an appropriate packaging signal. The present invention further provides the vector genome described above and templates that encode the same.

rAAV vectors useful in the methods of the disclosure are further described in PCT publication No. WO2015168666 and PCT publication no. WO2014011210, the contents of which are incorporated by reference herein.

### VI. Production of rAAV Vectors

Numerous methods are known in the art for production of rAAV vectors, including transfection, stable cell line production, and infectious hybrid virus production systems which include adenovirus-AAV hybrids, herpesvirus-AAV hybrids (Conway, J E et al., (1997). Virology 71(11):8780-8789) and baculovirus-AAV hybrids. rAAV production cultures for the production of rAAV virus particles all require: 1) suitable host cells, including, for example, human-derived cell lines such as HeLa, A549, or 293 cells, or insect-derived cell lines such as SF-9, in the case of baculovirus production systems: 2) suitable helper virus function, provided by wild-type or mutant adenovirus (such as temperature sensitive adenovirus), herpes virus, baculovirus, or a plasmid construct providing helper functions: 3) AAV rep and cap genes and gene products: 4) a transgene (such as a transgene comprising a nuclease system) flanked by at least one AAV ITR sequence: and 5) suitable media and media components to support rAAV production. Suitable media known in the art may be used for the production of rAAV vectors. These media include, without limitation, media produced by Hyclone Laboratories and JRH including Modified Eagle Medium (MEM), Dulbecco's Modified Eagle Medium (DMEM), custom formulations such as those described in U.S. Pat. No. 6,566,118, and Sf-900 II SFM media as described in U.S. Pat. No. 6,723,551, each of which is incorporated herein by reference in its entirety, particularly with respect to custom media formulations for use in production of recombinant AAV vectors.

The rAAV particles can be produced using methods known in the art. See, e.g., U.S. Pat. Nos. 6,566,118; 6,989,264: and 6,995,006. In practicing the disclosure, host cells for producing rAAV particles include mammalian cells, insect cells, plant cells, microorganisms and yeast. Host cells can also be packaging cells in which the AAV rep and cap genes are stably maintained in the host cell or producer cells in which the AAV vector genome is stably maintained. Exemplary packaging and producer cells are derived from 293, A549 or HeLa cells. AAV vectors are purified and formulated using standard techniques known in the art.

Recombinant AAV particles are generated by transfecting producer cells with a plasmid (cis-plasmid) containing a rAAV genome comprising a transgene flanked by the 145 nucleotide-long AAV ITRs and a separate construct expressing the AAV rep and CAP genes in trans. In addition, adenovirus helper factors such as E1A, E1B, E2A, E40RF6 and VA RNAs, etc. may be provided by either adenovirus infection or by transfecting a third plasmid providing adenovirus helper genes into the producer cells. Producer cells may be HEK293 cells. Packaging cell lines suitable for producing adeno-associated viral vectors may be readily accomplished given readily available techniques (see e.g., U.S. Pat. No. 5,872,005). The helper factors provided will vary depending on the producer cells used and whether the producer cells already carry some of these helper factors.

In some embodiments, rAAV particles may be produced by a triple transfection method, such as the exemplary triple transfection method provided infra. Briefly, a plasmid containing a rep gene and a capsid gene, along with a helper adenoviral plasmid, may be transfected (e.g., using the calcium phosphate method) into a cell line (e.g., HEK-293 cells), and virus may be collected and optionally purified.

In some embodiments, rAAV particles may be produced by a producer cell line method, such as the exemplary producer cell line method provided infra (see also (referenced in Martin et al., (2013) HUMAN GENE THERAPY METHODS 24:253-269). Briefly, a cell line (e.g., a HeLa cell line) may be stably transfected with a plasmid containing a rep gene, a capsid gene, and a promoter-transgene sequence. Cell lines may be screened to select a lead clone for rAAV production, which may then be expanded to a production bioreactor and infected with an adenovirus (e.g., a wild-type adenovirus) as helper to initiate rAAV production. Virus may subsequently be harvested, adenovirus may be inactivated (e.g., by heat) and/or removed, and the rAAV particles may be purified.

In some aspects, a method is provided for producing any rAAV particle as disclosed herein comprising (a) culturing a host cell under a condition that rAAV particles are produced, wherein the host cell comprises (i) one or more AAV package genes, wherein each said AAV packaging gene encodes an AAV replication and/or encapsidation protein: (ii) a rAAV pro-vector comprising a nucleic acid encoding a therapeutic polypeptide and/or nucleic acid as described herein flanked by at least one AAV ITR, and (iii) an AAV helper function: and (b) recovering the rAAV particles produced by the host cell. In some embodiments, said at least one AAV ITR is selected from the group consisting of AAV ITRs are AAV1, AAV2, AAV3, AAV4, AAV5, AAV6, AAV7, AAV8, AAVrh8, AAVrh8R, AAV9, AAV10, AAVrh10, AAV11, AAV 12, AAV2R471A, AAV DJ, a goat AAV, bovine AAV, or mouse AAV or the like. In some embodiments, the encapsidation protein is an AAV2 encapsidation protein.

Suitable rAAV production culture media of the present disclosure may be supplemented with serum or serum-derived recombinant proteins at a level of 0.5-20 (v/v or w/v). Alternatively, as is known in the art, rAAV vectors may be produced in serum-free conditions which may also be referred to as media with no animal-derived products. One of ordinary skill in the art may appreciate that commercial or custom media designed to support production of rAAV vectors may also be supplemented with one or more cell culture components know in the art, including without limitation glucose, vitamins, amino acids, and or growth factors, in order to increase the titer of rAAV in production cultures.

rAAV production cultures can be grown under a variety of conditions (over a wide temperature range, for varying lengths of time, and the like) suitable to the particular host cell being utilized. As is known in the art, rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

rAAV vector particles of the disclosure may be harvested from rAAV production cultures by lysis of the host cells of the production culture or by harvest of the spent media from the production culture, provided the cells are cultured under conditions known in the art to cause release of rAAV particles into the media from intact cells, as described more fully in U.S. Pat. No. 6,566,118). Suitable methods of lysing cells are also known in the art and include for example multiple freeze/thaw cycles, sonication, microfluidization, and treatment with chemicals, such as detergents and/or proteases.

In a further embodiment, the rAAV particles are purified. The term “purified” as used herein includes a preparation of rAAV particles devoid of at least some of the other components that may also be present where the rAAV particles naturally occur or are initially prepared from. Thus, for example, isolated rAAV particles may be prepared using a purification technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In some embodiments, the rAAV production culture harvest is clarified to remove host cell debris. In some embodiments, the production culture harvest is clarified by filtration through a series of depth filters including, for example, a grade DOHC Millipore Millistak+HC Pod Filter, a grade AIHC Millipore Millistak+HC Pod Filter, and a 0.2 uvn Filter Opticap XL 10 Millipore Express SHC Hydrophilic Membrane filter. Clarification can also be achieved by a variety of other standard techniques known in the art, such as, centrifugation or filtration through any cellulose acetate filter of 0.2 uvn or greater pore size known in the art.

In some embodiments, the rAAV production culture harvest is further treated with Benzonase R to digest any high molecular weight DNA present in the production culture. In some embodiments, the Benzonase R digestion is performed under standard conditions known in the art including, for example, a final concentration of 1-2.5 units/ml of Benzonase R at a temperature ranging from ambient to 37° ° C. for a period of 30 minutes to several hours.

rAAV particles may be isolated or purified using one or more of the following purification steps: equilibrium centrifugation: flow-through anionic exchange filtration: tangential flow filtration (TFF) for concentrating the rAAV particles: rAAV capture by apatite chromatography: heat inactivation of helper virus: rAAV capture by hydrophobic interaction chromatography: buffer exchange by size exclusion chromatography (SEC): nanofiltration: and rAAV capture by anionic exchange chromatography, cationic exchange chromatography, or affinity chromatography. These steps may be used alone, in various combinations, or in different orders. In some embodiments, the method comprises all the steps in the order as described below. Methods to purify rAAV particles are found, for example, in Xiao et al., (1998) Journal of Virology 72:2224-2232: U.S. Pat. Nos. 6,989,264 and 8,137,948; and WO 2010/148143.

### VII. Pharmaceutical Compositions

Also provided herein are pharmaceutical compositions comprising a nuclease system described herein and a pharmaceutically acceptable carrier. The pharmaceutical compositions may be suitable for any mode of administration described herein.

In some embodiments, the pharmaceutical compositions comprising a nucleic acid described herein and a pharmaceutically acceptable carrier is suitable for administration to a human subject. Such carriers are well known in the art (see, e.g., Remington's Pharmaceutical Sciences, 15th Edition, pp. 1035-1038 and 1570-1580). Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multidosage forms. The compositions are generally formulated as sterile and substantially isotonic solution.

In one embodiment, the nucleic acid comprising the nuclease system and compact bidirectional promoter for use in the target cells as detailed above is formulated into a pharmaceutical composition intended for oral, inhalation, intranasal, intratracheal, intravenous, intramuscular, subcutaneous, intradermal, and other parental routes of administration. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. Publication No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid). Routes of administration may be combined, if desired.

The composition may be delivered in a volume of from about 0.1 μL to about 1 mL, including all numbers within the range, depending on the size of the area to be treated, the viral titer used, the route of administration, and the desired effect of the method. In one embodiment, the volume is about 50 μL. In another embodiment, the volume is about 70 μL. In a preferred embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. An effective concentration of a recombinant adeno-associated virus carrying a nucleic acid sequence encoding the desired transgene under the control of the cell-specific promoter sequence desirably ranges from about 10⁷ and 10¹³ vector genomes per milliliter (vg/mL) (also called genome copies/mL (GC/mL)). The rAAV infectious units are measured as described in S. K. McLaughlin et al., 1988 J. Virol., 62: 1963, which is incorporated herein by reference.

Preferably, the concentration in the target tissue is from about 1.5×10⁹ vg/mL to about 1.5×10¹² vg/mL, and more preferably from about 1.5×10⁹ vg/mL to about 1.5×10¹¹ vg/mL. In certain preferred embodiments, the effective concentration is about 2.5×10¹⁰ vg to about 1.4×10¹¹. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. In one embodiment, the effective dosage (total genome copies delivered) is from about 10⁷ to 10¹³ vector genomes. It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed.

Pharmaceutical compositions useful in the methods of the disclosure are further described in PCT publication No. WO2015168666 and PCT publication no. WO201401 1210, the contents of which are incorporated by reference herein.

### VIII. Kits

In some embodiments, any of the vectors disclosed herein is assembled into a pharmaceutical or diagnostic or research kit to facilitate their use in therapeutic, diagnostic or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a +10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1. Therapeutic Development of Compact Promoters for Expression of Nuclease

### Systems

This Example describes identification and characterization of a promoter that is small, strong, ubiquitous, and endogenous, for adeno-associated virus (AAV) packaging of nuclease systems.

Bioinformatics analysis revealed the H1 bidirectional promoter appears to be ubiquitously expressed, which is logical given the biology and tissue expression data for both H1-driven genes (H1RNA and PARP-2). Endogenously, the H1 bidirectional promoter expresses an essential RNA gene (H1RNA) involved with tRNA processing and a ubiquitously expressed protein gene (PARP2). While a lack of transgene silencing using the H1 bidirectional promoter is not guaranteed, this result would be consistent with other endogenous mammalian promoters.

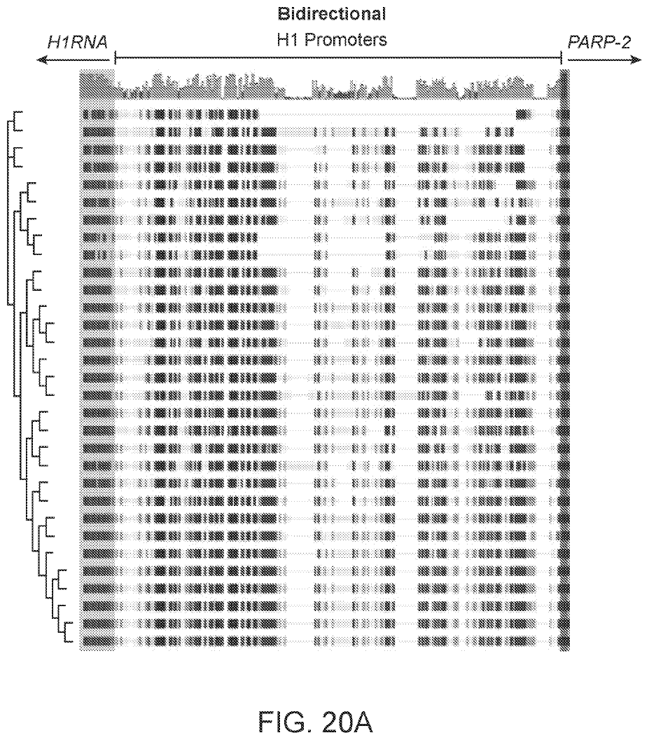

Evolutionary conservation throughout eutherian mammals further supports the presence of a functional genetic regulatory element between the H1RNA and PARP2 genes, and enabled identification of numerous small and compact promoters through gene synteny (FIG. 20A). The orthologous H1 bidirectional promoters tested have all shown promoter activity in human cell lines, as well as cell lines of multiple different species.

To test the relative strength of the numerous promoter orthologs, a luciferase reporter construct that enables quantitation of RNA polymerase II (pol II) promoter activity was designed. In order to reduce any confounding noise and spurious reporter gene transcription, the plasmid constructs contained 5′ and 3′ beta-globin insulators that flank the expression cassette: the H1 promoter, firefly luciferase, and bGH poly(A) signal were found inside the insulators. It was observed that the pol II promoter activity varied significantly between orthologs, and consequently, the analysis was expanded to over 70 promoters, each tested in multiple human cell lines (FIG. 20B). The constructs were fully-synthesized, sequence verified, and amplified by endotoxin-free maxipreps for transfection studies.

In order to benchmark the pol II expression levels of these H1 promoters against known promoters, two commonly used promoters were included, the HSK thymidine kinase (TK) promoter and the phosphoglycerate kinase 1 (PGK1) promoter. The TK promoter is 753 basepairs (bp) and known to be a promoter that drives lower expression levels of regulated genes, while PGK1 is 515 bp and known to drive higher expression of regulated genes. The data in FIG. 20B shows the ranked order of promoter activity in Hela cells with TK (orange, 8th bar from the left) and PGK1 (blue, 1st bar from the right) indicated. FIG. 20B demonstrates a wide range of expression of the H1 promoter orthologs.

Additionally, the promoter lengths were plotted overlaying the same data with red bars and corresponding to the right Y axis (a non-standard Y-axis range of 150 bp to 250 bp was used to depict the sizes for each promoter clearly). In addition to a range of activity, the promoter sizes were small (between about 150-240 bp) and demonstrated no correlation between size and promoter activity. Indeed, multiple promoters were found in the 150-180 bp size range with significant transcriptional activity. Nine of the promoters were 183 bp or smaller.

### Example 2. Mouse H1 Promoter Deletion Analysis

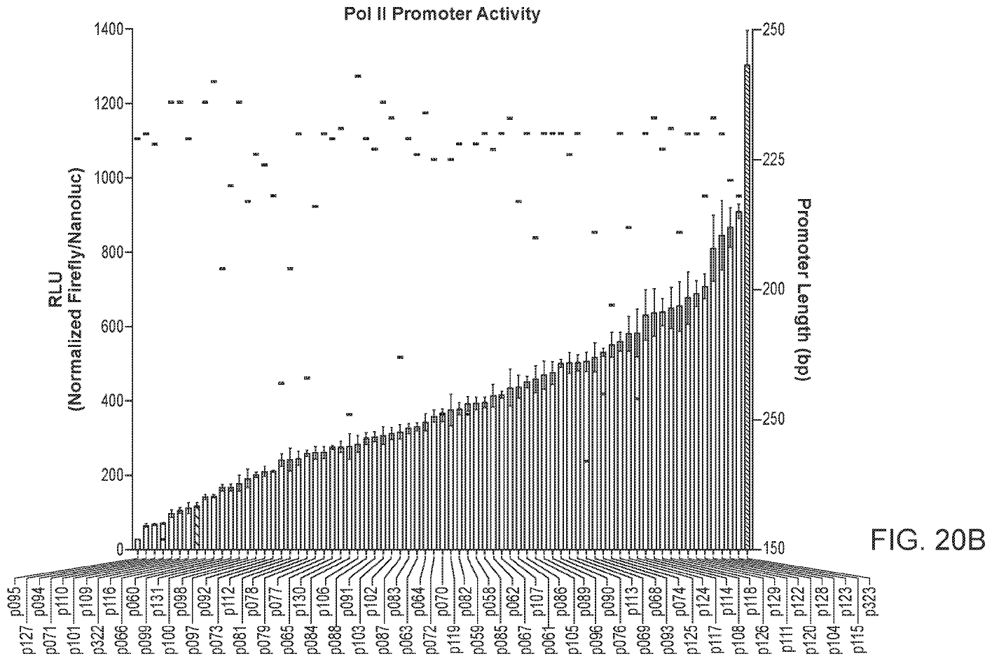

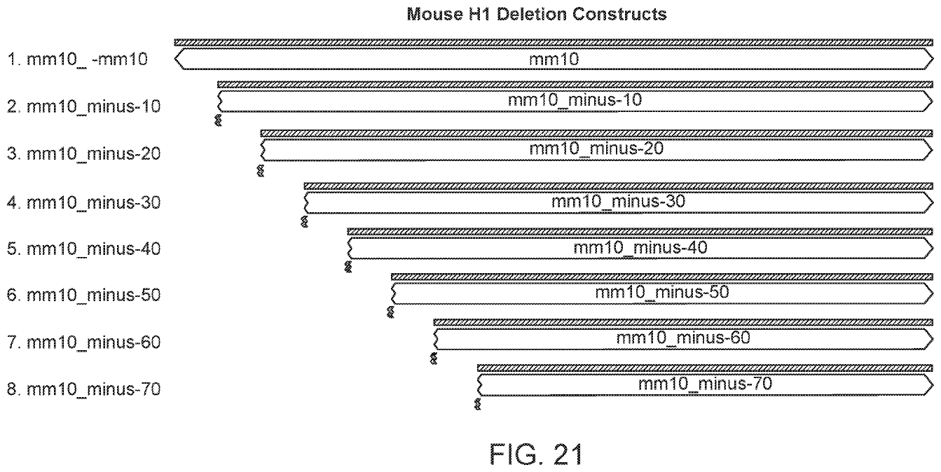

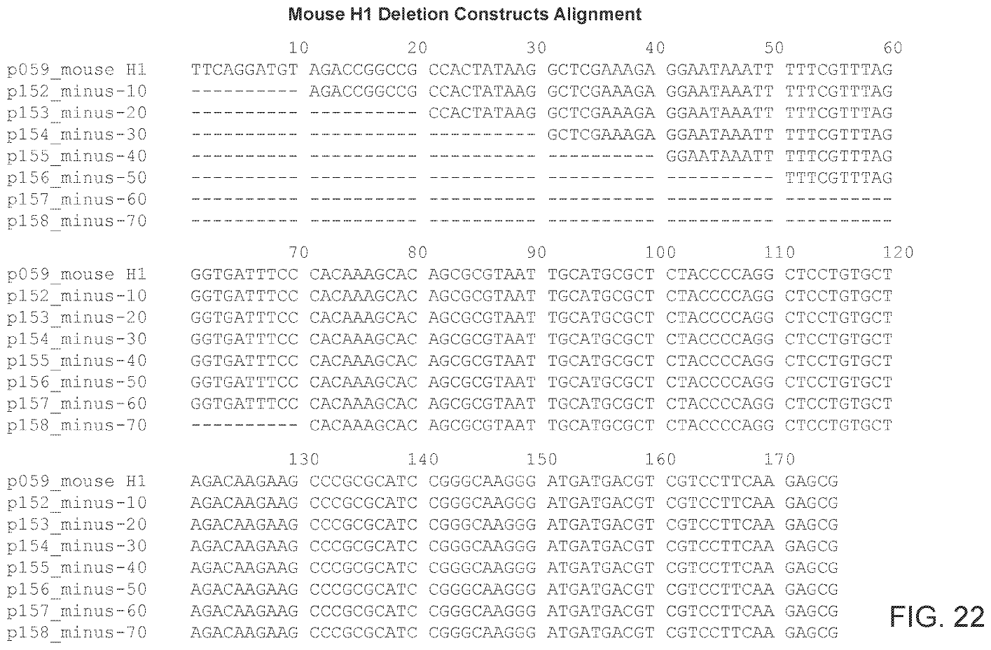

To determine which regions of the mouse H1 promoter were need for activity, a series of mouse H1 promoter constructs were made and tested. A schematic representation of the mouse H1 promoter deletion constructs is shown in FIG. 21, with the wild-type mouse promoter (p059, SEQ ID NO: 93) shown at the top and seven successive 10 bp deletion constructs shown below: An alignment of the various deletion constructs is provided in FIG. 22. These promoters and variants were used to drive reporters and quantitate expression.

To test the relative activity of promoters, luciferase reporter constructs were designed that enable quantitation of the Pol II promoter activity of the promoters. To reduce confounding noise and spurious reporter gene transcription, the plasmid constructs contain 5′ and 3′ beta-globin insulators that flank the expression cassette: the promoter sequence connected to a control guide RNA on one side and firefly luciferase on the other side, and bGH poly(A) signal are found inside the insulators.

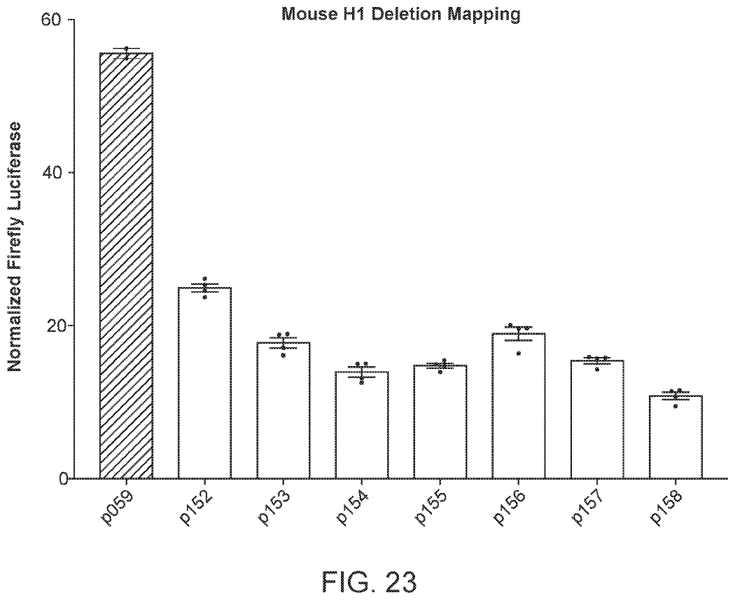

Generally, cell lines were subcultured and seeded into 96-well plates 24 hours prior to transfection. On the day of transfection, the firefly luciferase construct was co-transfected with the NanoLuc control construct using Lipofectamine 3000. At 24 hours post-transfection, plates were sequentially assayed for firefly luciferase and NanoLuc using the Nano-Glo Dual-Luciferase Reporter Assay System (Promega) by imaging for total luminescence on a plate reader (Biotek). For data analysis and plotting, the firefly luminescence signal was normalized to the control Nanoluc signal in each well. Technical replicates within samples were averaged together to produce a single biological replicate value, and the mean values between biological replicates were then plotted with error bars indicating the SEM. Results are shown in FIG. 23 (normalized firefly to nanoluc luciferase signal for each construct).

As shown in FIG. 23, each deletion construct retained a portion of the full-length wild-type H1 promoter activity. It is contemplated that fragments of H1 promoters (e.g., the H1 promoters described herein) that retain activity can be used to express a nuclease system, for example, that includes both a nuclease and a gRNA.

### Example 3. Mouse H1 Promoter Mutation Analysis

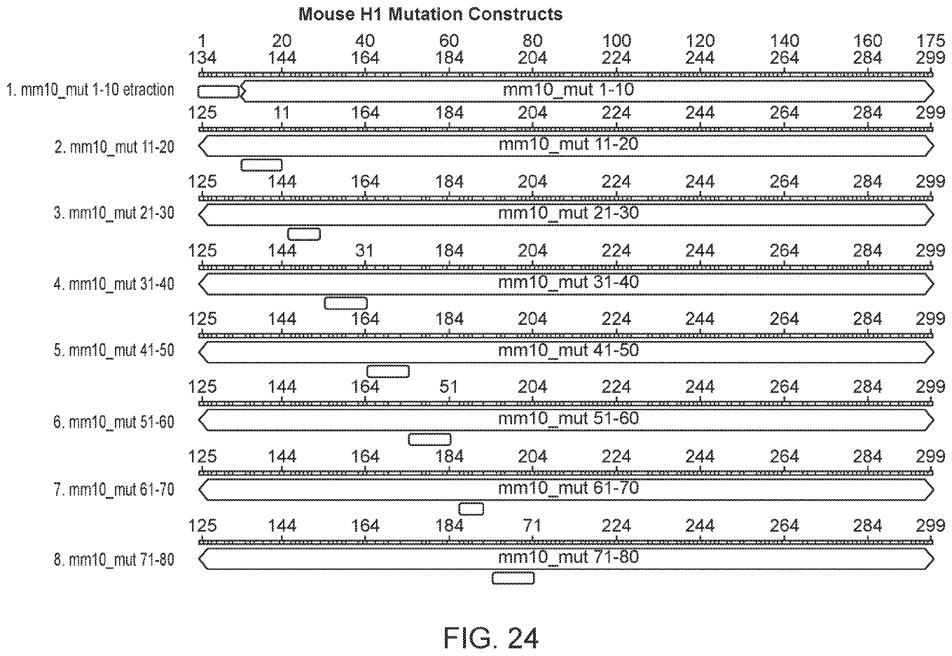

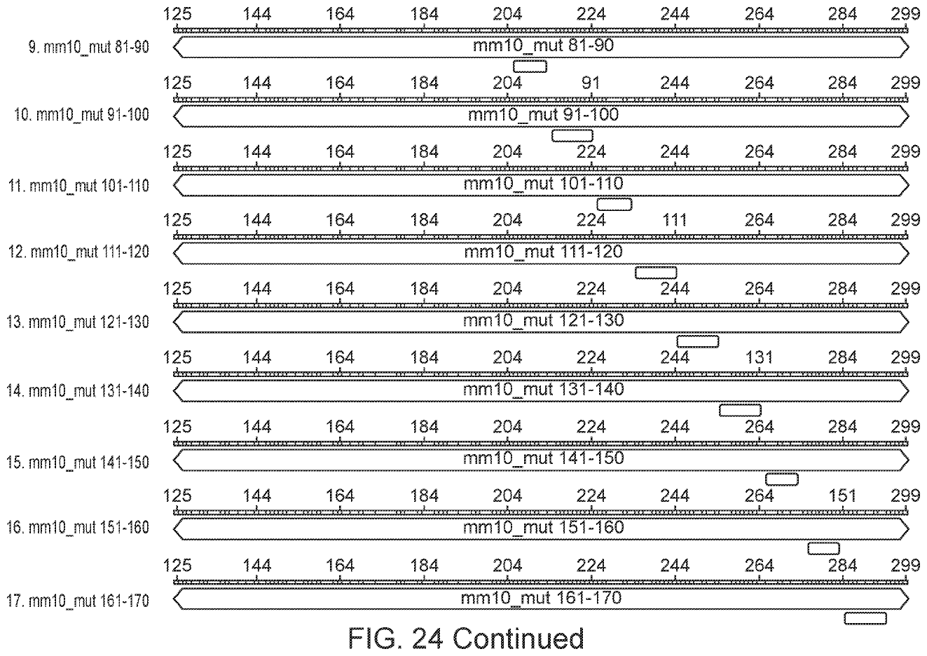

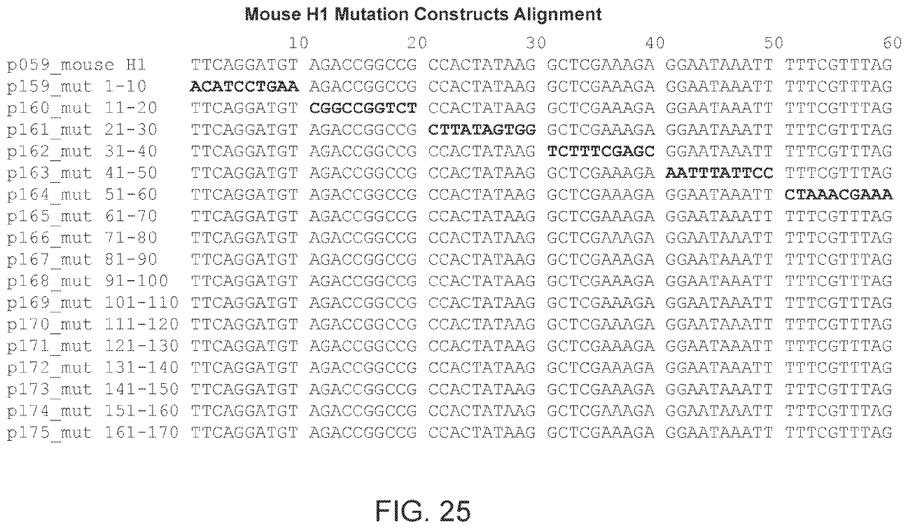

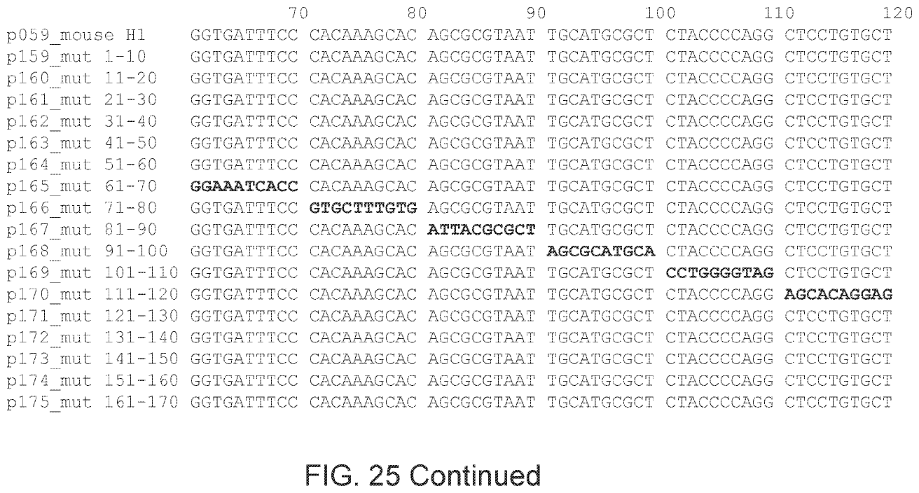

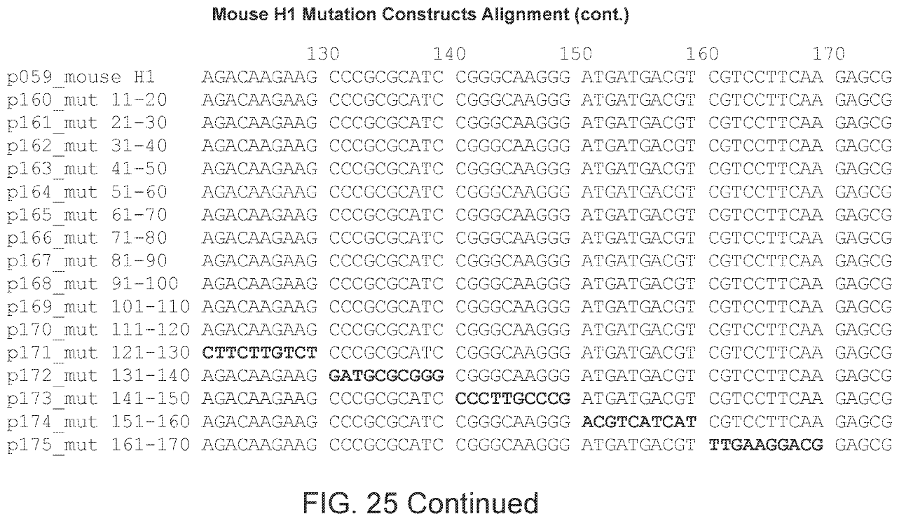

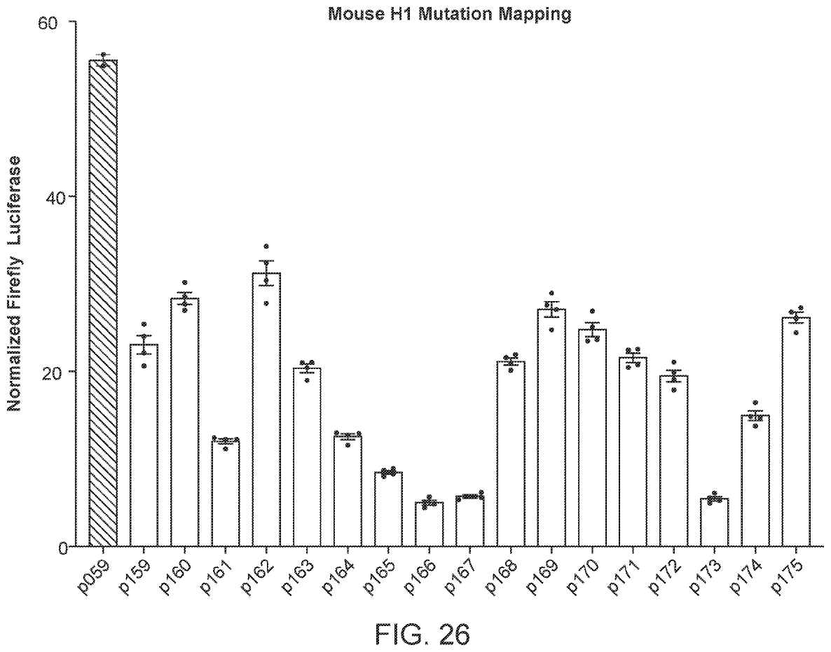

Seventeen (17) mutation constructs were designed by walking across the promoter in 10 bp increments and replacing the sequence with its reverse complement. A schematic representation of the constructs is shown in FIG. 24 and an alignment of the sequences shown in FIG. 25. Constructs were made and tested as described in Example 2. Results are shown in FIG. 26.

As shown in FIG. 26, each mutation construct retained a portion of the full-length wild-type H1 promoter activity. It is contemplated that variants of H1 promoters (e.g., the H1 promoters described herein) that retain activity can be used to express a nuclease system, for example, that includes both a nuclease and a gRNA.

### Example 4. Mouse H1 Promoter with Introns

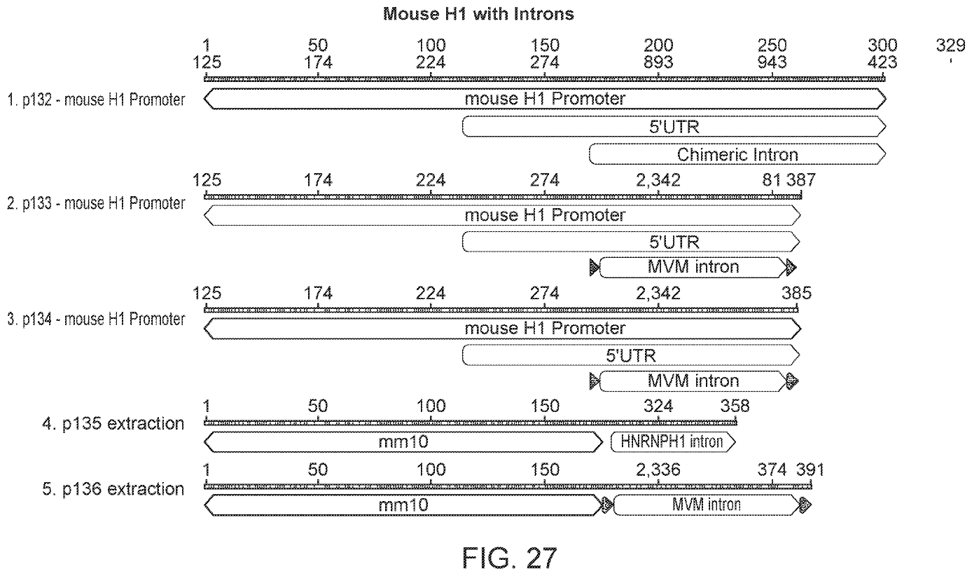

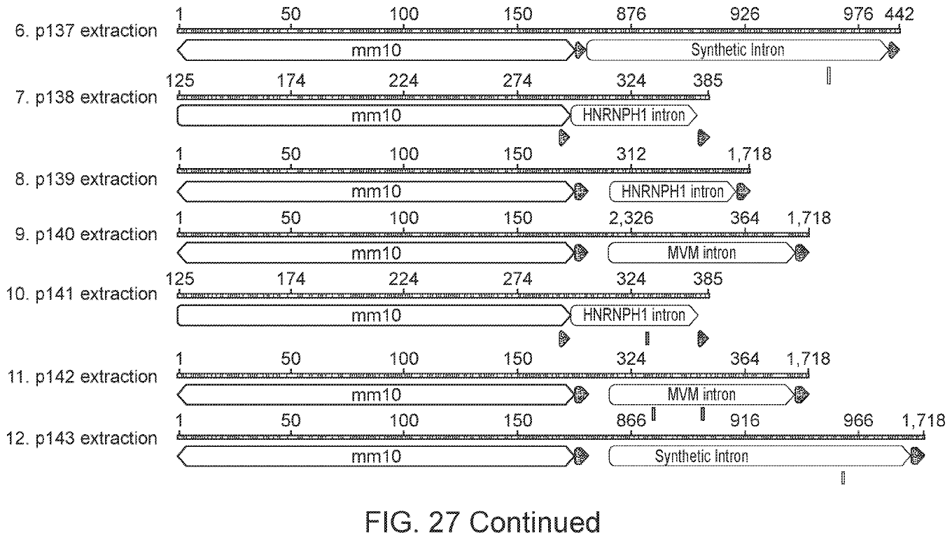

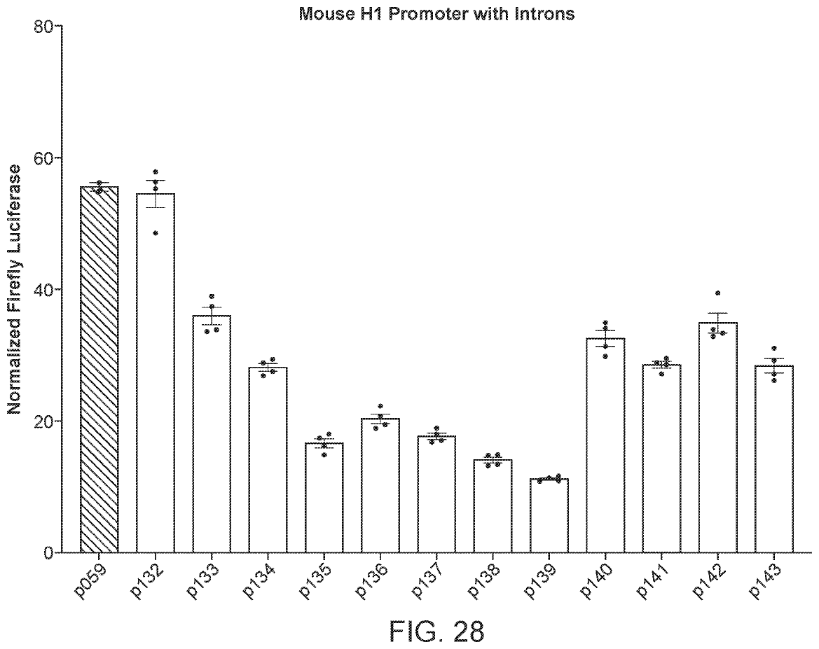

Twelve (12) different constructs were designed to incorporate introns into the mouse H1 promoter region. Different intron sequences and different insertion locations were used as shown in FIG. 27. Constructs were made and tested as described in Example 2. Results are shown in FIG. 28.

As shown in FIG. 28, each intron construct retained at least a portion of the full-length wild-type H1 promoter activity. It is contemplated that variants (e.g., intron-containing variants) of H1 promoters (e.g., the H1 promoters described herein) that retain activity can be used to express a nuclease system, for example, that includes both a nuclease and a gRNA.

### Example 5. Human and Mouse H15′UTR Constructs

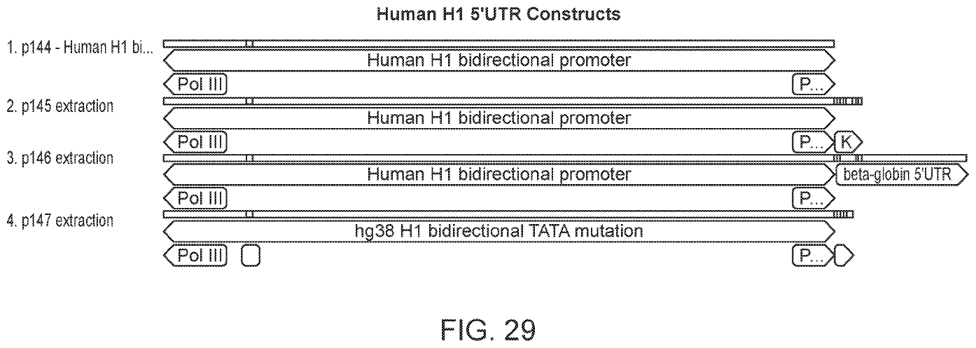

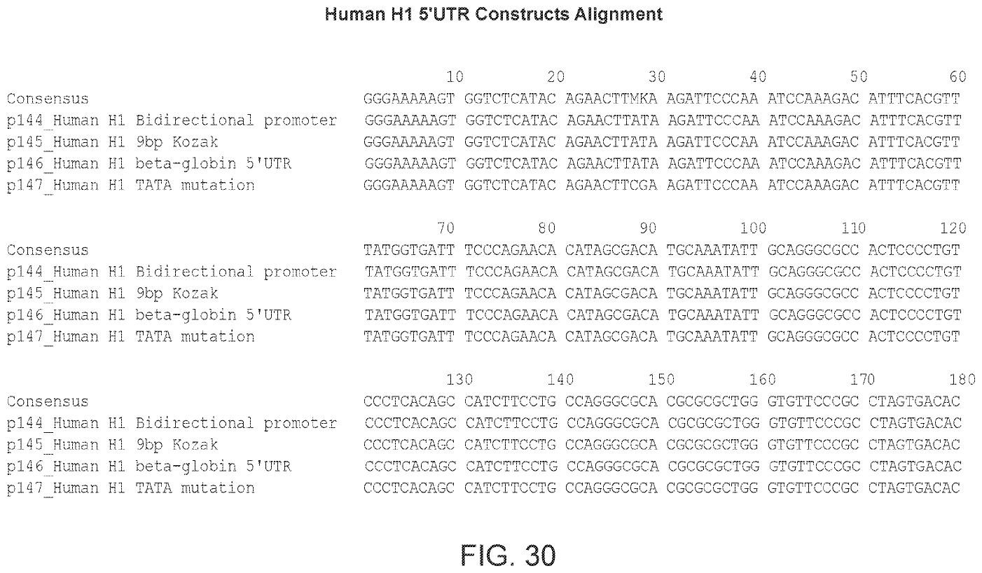

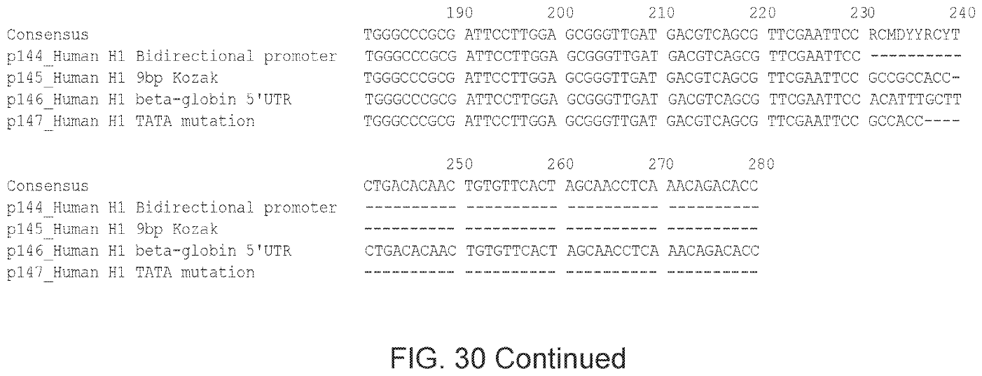

FIG. 29 provides a schematic showing the design of human H1 promoter and variant constructs. As shown in FIG. 29, a construct carrying a human H1 promoter alone, a human H1 promoter with a 9 bp Kozak sequence (GCCGCCACC (SEQ ID NO: 256)), a human H1 promoter with a beta-globin 5′UTR, and a human H1 promoter with a TATA box mutation (TATAA->TCGAA) were designed. An alignment of the sequences is shown in FIG. 30.

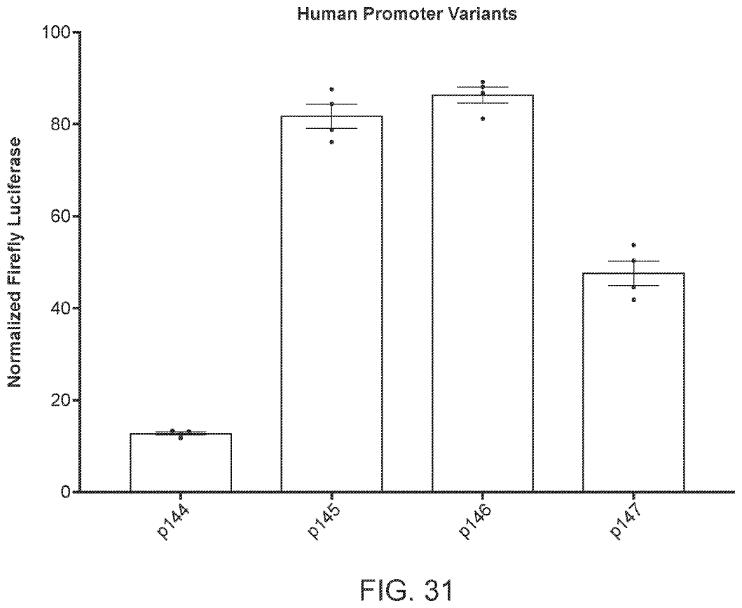

Constructs were made and tested as described in Example 2. Results are shown in FIG. 31.

As shown in FIG. 31 addition of 5′UTR sequences increased expression from an H1 promoter. Accordingly, such 5′UTR sequences can be used to increase expression from a promoter as described herein (e.g., an H1 promoter).

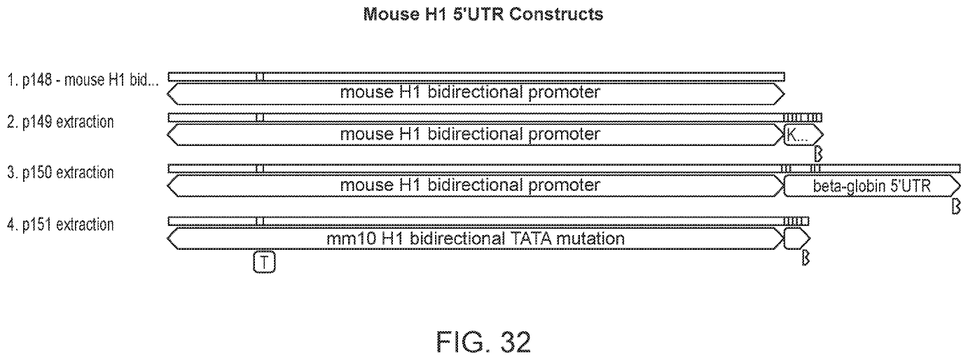

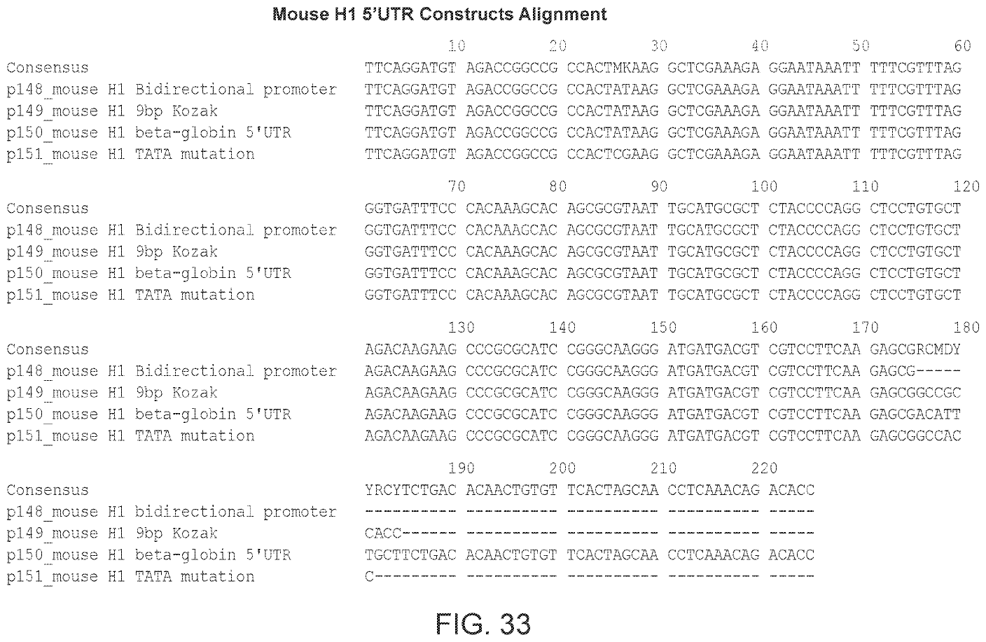

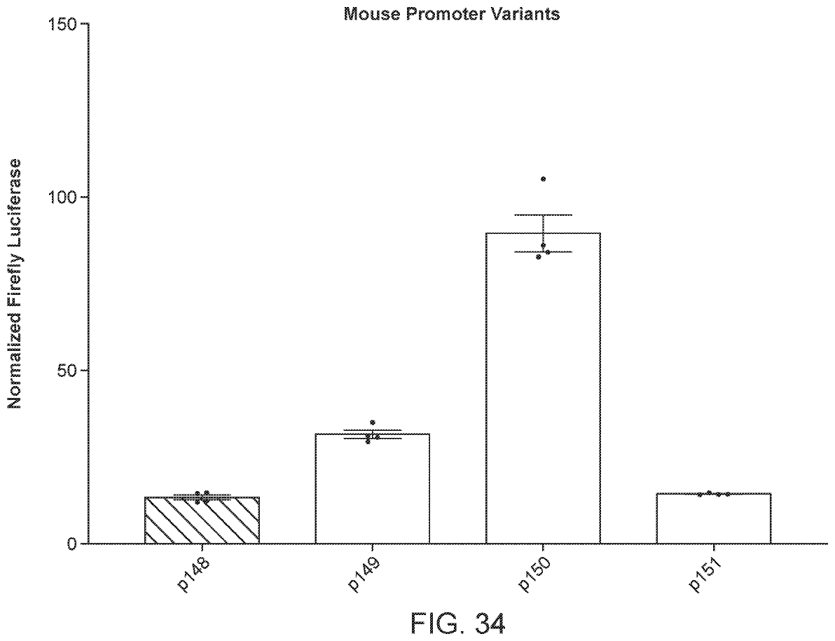

H1 5′UTR constructs also were made and tested using the mouse H1 promoter, as shown in FIGS. 32 and 33. Results are shown in FIG. 34.

As shown in FIG. 34, most of the tested 5′UTR sequences increased expression from a mouse H1 promoter. Accordingly, such 5′UTR sequences can be used to increase expression from a promoter as described herein (e.g., a mouse H1 promoter).

### Example 6. Expression of H1, Gar-1 and Other Bidirectional Promoters

Additional constructs were designed as described above, but using the following promoters: human H1 (p144: SEQ ID NO: 87), mouse H1 (p148: SEQ ID NO: 93), human 7sk-1 (p199: SEQ ID NO: 242), mouse 7sk-1 (p203: SEQ ID NO: 204), human ALOXE3 (p204: SEQ ID NO: 246), human CGB1 (p206: SEQ ID NO: 247), human CGB2 (p207: SEQ ID NO: 248), human GAR1-1 (p216: SEQ ID NO: 107), human Med16-1 (p222: SEQ ID 0 NO: 249), human Med16-2 (p223: SEQ ID NO: 250), human SRP (p242: SEQ ID NO: 233).

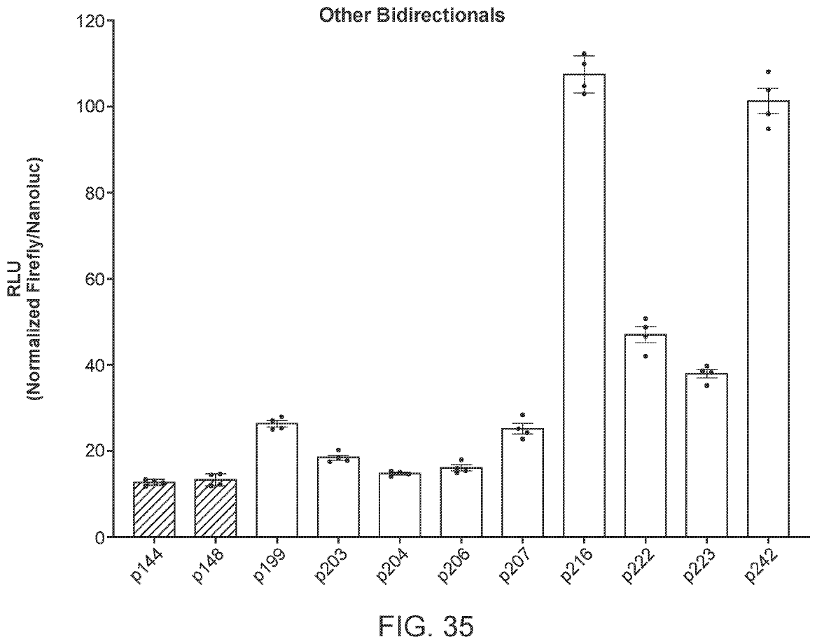

Constructs were made and tested as described above. Results are shown in FIG. 35.

As shown in FIG. 35, most of the tested bidirectional promoters showed increased expression as compared to an H1 promoter. Gar-1 showed the highest level of expression. Accordingly, such compact bidirectional promoters can be used to express a nuclease system using a vector, such as an AAV vector, that has limited space. 15

### Example 7. Assessment of Promoter Activity in Exemplary Cell Lines

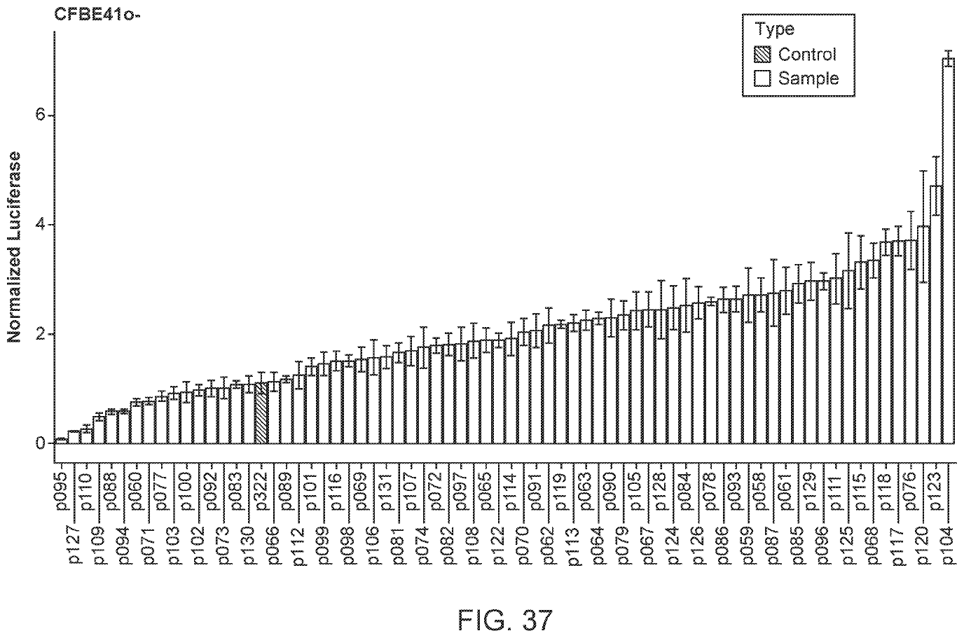

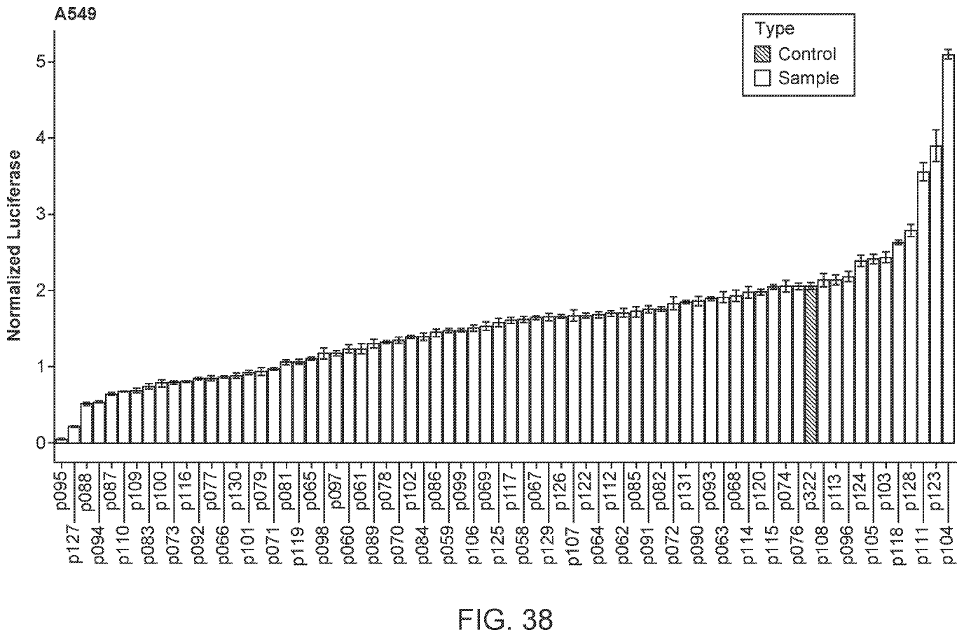

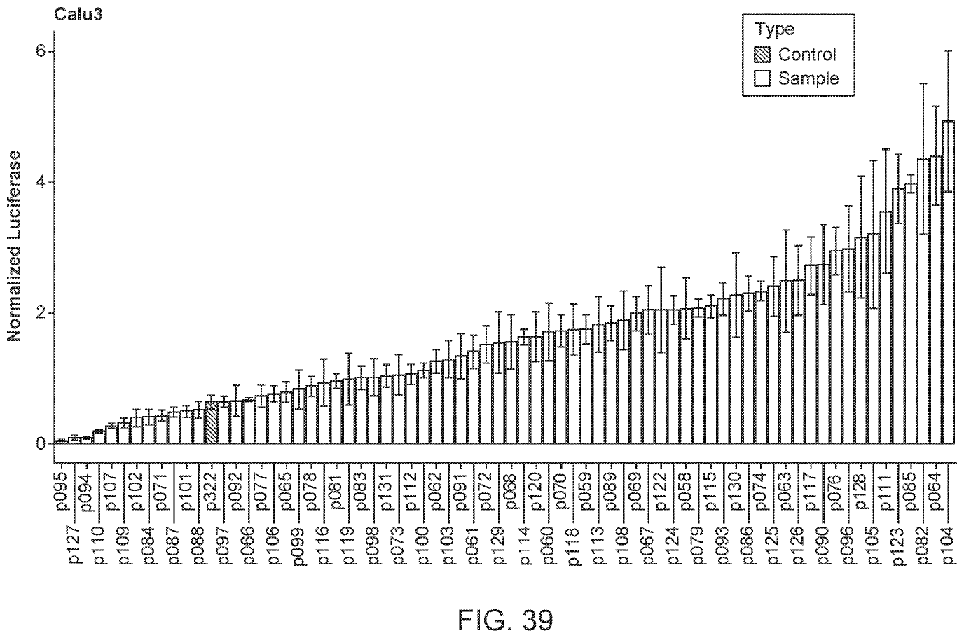

This Example describes the characterization of a library of H1 promoters for their capacity to drive gene expression using luciferase reporters (Firefly luciferase and NANOLUCR) in three lung cell lines (A549, Calu-3, and CFBE410-). Normalized luciferase expression was quantified for 71 H1 promoters and benchmarked against a control thymidine kinase (TK) promoter (FIGS. 37, 38, and 39).

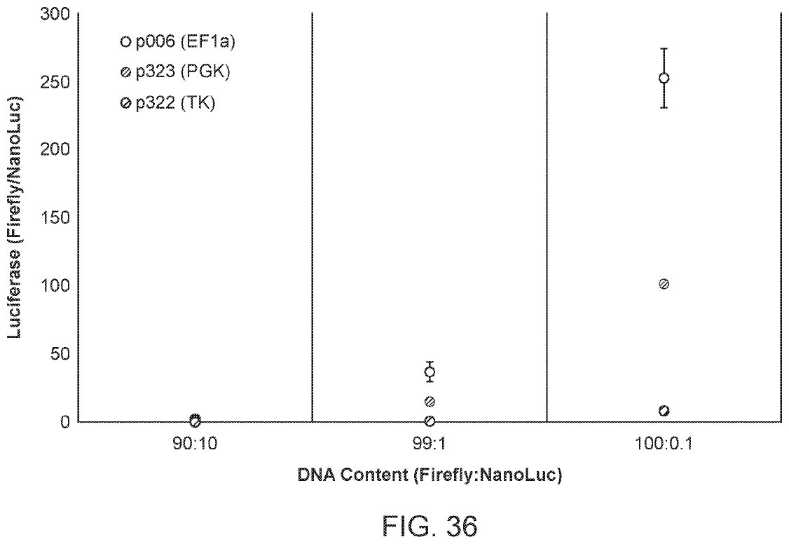

Promoter expression activity was assessed using a luciferase reporter assay. Characterization of the luciferase assay was performed by co-transfecting cells with a plasmid encoding Firefly luciferase and with a plasmid encoding NANOLUCR reporters. The luciferase reporters were under transcriptional control of standard promoters (EF1a, PGK, and TK). A standard curve of the normalized luciferase signal (Firefly signal/NANOLUCR signal) was generated using the following transfection ratios, 90 ng Firefly: 10 ng NANOLUCR, 99 ng Firefly: 1 ng NANOLUCR, and 100 ng Firefly:0. 1 ng NANOLUCR (FIG. 36). Establishing such a ratiometric luciferase reporter assay allowed the determination of promoter expression activity without cross-signal interference.

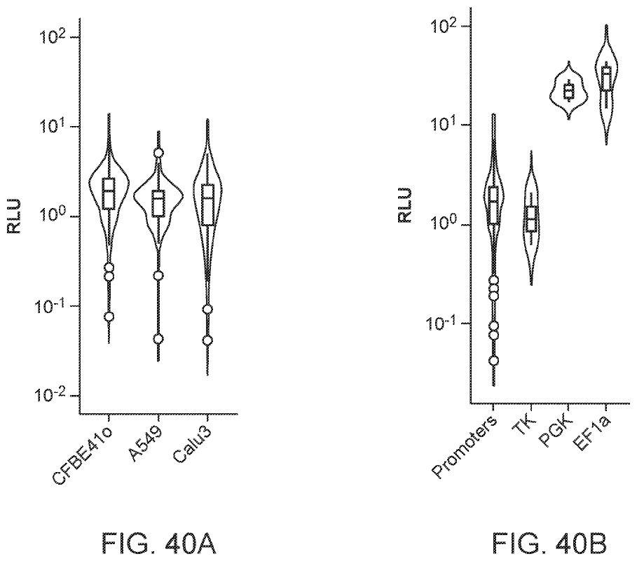

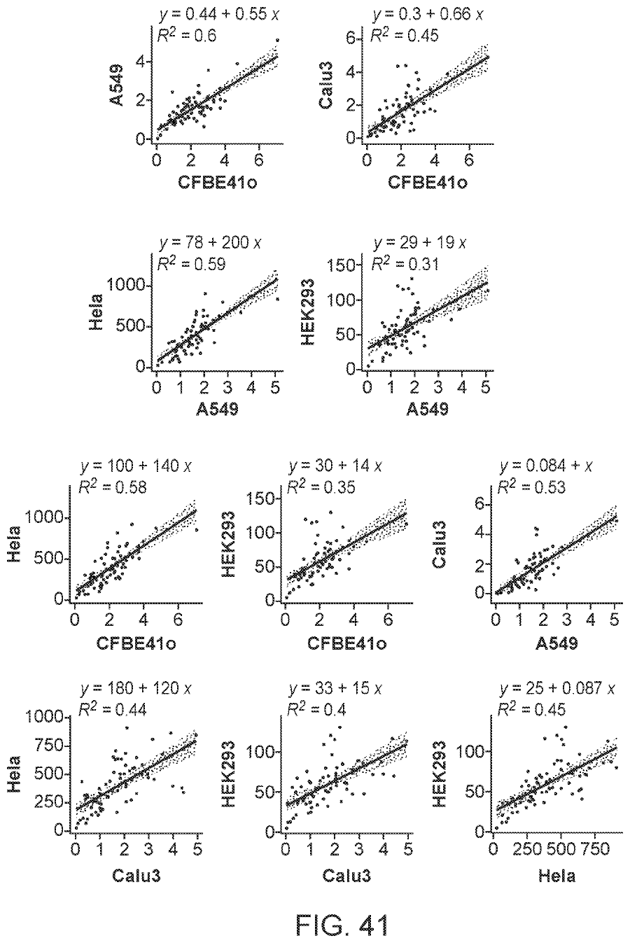

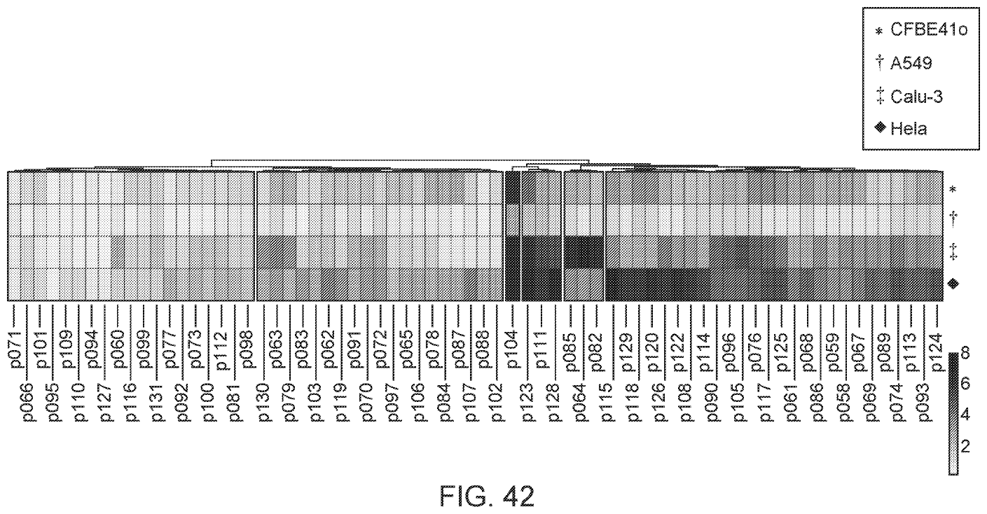

A library of 71 H1 promoters was then evaluated for expression activity in three lung cell types (A549, Calu-3, and CFBE410-) (FIGS. 37, 38, and 39) and two non-lung cell types (HEK293 and HeLa) used as control samples. Rank-order activity of the compact promoters in the library is shown in FIGS. 37, 38, and 39, along with activity of the standard TK promoter is shown (“TK”). Distributions of expression activity across the three lung cell types is shown in FIG. 40A. Of the 71 compact H1 promoters tested, 59 promoters in Calu-3 cells, 55 promoters in CFBE410-cells, and 11 in A549 cells exceeded TK controlled expression of luciferase reporter plasmids. The strongest promoters exceeded TK controlled expression activity by 2.5-8-fold and were only modestly weaker than the two strong standard promoters PGK and EF1a (FIG. 40B). The data suggests that most of the H1 promoters are active in lung cell lines. Furthermore, the promoters in this library do not contain viral or synthetic elements that can have negative consequences stemming from long-range enhancer activity. The data also showed that promoter activity was well-correlated among lung cell lines and across non-lung-cell types (FIG. 41). Hierarchical analysis (complete linkage clustering) was conducted to produce a heatmap as shown in FIG. 42. Through hierarchical analysis, a pattern suggesting that strong promoters in one cell type are likely to be strong promoters in other cell types emerged, enabling the clustering of promoters based on expression activity into six separate clusters (FIG. 42). Cluster 1 included promoters p071, p066, p101, p095, p109, p110, p094, p127, p060, p116, p099, p131, p077, p092, p073, p100, p112, p081, and p098. Cluster 2 included promoters p130, p063, p079, p083, p103, p062, p119, p091, p070, p072, p097, p065, p106, p078, p084, p087, p107, p088, and p102. Cluster 3 included promoter p104. Cluster 4 included promoters p123, p111, and p128. Cluster 5 included promoters p085, p064, and p082. Cluster 6 included promoters p115, p129, p118, p120, p126, p122, p108, p114, p090, p096, p105, p076, p117, p125, p061, p068, p086, p059, p058, p067, p069, p089, p074, p113, p093, and p124. Clusters 3-6 showed higher expression levels above the control TK p322 promoter.

Following clustering based on expression activity, the top five and bottom five promoters in A549 cells were identified, along with their respective ranking in four other cell types, as shown in TABLE 35.

TABLE 35
The top five and bottom five promoters in A549,
CFBE41o-, Calu-3, HeLa, and HEK293 cells.

| | | | | |
|---|---|---|---|---|
| A549 | CFBE41o- | Calu-3 | HeLa | HEK293 |

Top five promoters

| | | | | | |
|---|---|---|---|---|---|
| p104 | 1 | 1 | 1 | 3 | 5 |
| p123 | 2 | 2 | 5 | 2 | 10 |
| p111 | 3 | 10 | 6 | 7 | 20 |
| p128 | 4 | 24 | 8 | 4 | 11 |
| p118 | 5 | 6 | 31 | 10 | 23 |

Bottom five promoters

| | | | | | |
|---|---|---|---|---|---|
| p087 | 67 | 15 | 62 | 41 | 25 |
| p094 | 68 | 66 | 69 | 69 | 60 |
| p088 | 69 | 67 | 60 | 45 | 54 |
| p127 | 70 | 70 | 70 | 70 | 70 |
| p095 | 71 | 71 | 71 | 71 | 71 |

Wild type AAV genomes are ˜4.7 kb in length and recombinant AAV can package up to ˜5.2 kb. Given that AAV packaging efficiency may improve with smaller cassettes, a subset of promoters <200 bp was further analyzed and ranked as shown in TABLE 36.

TABLE 36
Ranked expression for ultra-compact (≤200 bp) promoters.
Ranked Expression

| | CFBE41o- | A549 | Calu-3 | HeLa | HEK293 | Size (bp) |
|---|---|---|---|---|---|---|
| p074 | 43 | 13 | 16 | 16 | 13 | 197 |
| p093 | 18 | 19 | 19 | 17 | 1 | 180 |
| p117 | 5 | 35 | 12 | 13 | 46 | 179 |
| p069 | 48 | 37 | 26 | 19 | 4 | 167 |
| p059 | 17 | 40 | 30 | 33 | 42 | 176 |

The compact promoters described herein are advantageous for their ability to drive expression of a protein and an RNA, such a nuclease and a guide RNA, while allowing packaging in an AAV vector, circumventing long-standing challenges with AAV vector use for gene editing applications. Many of the compact promoters described herein show expression levels at least as strong as a TK promoter (see, e.g., FIG. 40B).

### Example 8. Generation of Ancestral H1 Promoter Sequences

This example describes the generation of synthetic H1 promoters (SEQ ID NOs: 936-1303) by reconstructing ancestral sequences from the H1 promoters herein described (e.g., SEQ ID NOs: 25-106, 469-476, 559-564, 609-614, 673-678, 681, 692-697, 706-711, 719-724, 729-734, 748-753, 784-789, 904-909, and 920-925).

First, a phylogenetic tree was built using RAxML or MEGA, as described in A. Stamatakis: “RAXML Version 8: A tool for Phylogenetic Analysis and Post-Analysis of Large Phylogenies” In Bioinformatics, 2014; Nei M. and Kumar S. (2000) Molecular Evolution and Phylogenetics Oxford University Press, New York: Tamura K., Stecher G., and Kumar S. (2021) MEGA 11: Molecular Evolutionary Genetics Analysis Version 11 Molecular Biology and Evolution https://doi.org/10.1093/molbev/msab120; and Stecher G., Tamura K., and Kumar S (2020) Molecular Evolutionary Genetics Analysis (MEGA) for macOS Molecular Biology and Evolution 37:1237-1239, herein incorporated by reference in their entireties.

For analysis with MEGA, the evolutionary history was inferred by using the Maximum Likelihood method and General Time Reversible model. The tree with the highest log likelihood (-25977.38) was selected. Initial tree(s) for the heuristic search were obtained automatically by applying Neighbor-Join and BioNJ algorithms to a matrix of pairwise distances estimated using the Maximum Composite Likelihood (MCL) approach, and then selecting the topology with superior log likelihood value. A discrete Gamma distribution was used to model evolutionary rate differences among sites (5 categories (+G, parameter=0.9471)). The rate variation model allowed for some sites to be evolutionarily invariable ([+I], 0.30% sites). This analysis involved 408 nucleotide sequences. There were a total of 467 positions in the final dataset. Evolutionary analyses were conducted in MEGA11.

The phyloFit program from PHAST (Phylogenetic Analysis with Space/Time Models) package was used to generate a phylogenetic model by fitting the tree models to the multiple sequence alignment by maximum likelihood using the HKY85 substitution model. The PREQUEL (Probabilistic REconstruction of ancestral seQUEnces, Largely) program from PHAST was used to compute marginal probability distributions for bases at ancestral nodes in the phylogenetic tree, using the tree model defined by phyloFit. Distributions were computed using the sum-product algorithm, assuming independence of sites. The identified sequences (SEQ ID NOs: 936-1303) correspond to nodes in the original tree.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

### SEQUENCE LISTING

H1 Sequences:
>Aardvark_H1_Bidirectional_Promoter
(SEQ ID NO: 25)
GGAACGAAACTAACTTGGCCAAACTATATAAGAATGCCATAGCTTTCAACATTTAATGGTTAGGGTGCCTTCTCA
TAATACACAGCGACATGCAAATATCATGGCCCTTCCAGGAGGCGTGCCTCCCCGTCCCGCGTGTGCGTCTTGCTT
GTGCGCAGGCGCGCTGCTCTTCCGGCTGTAAGACTTTGAGCCCTTGATTTCTGTGAGCGGGTTCGTGAAGTCAGT
GTTCTGGCTCC
>Angolan_colobus_H1_Bidirectional_Promoter
(SEQ ID NO: 26)
GGGGAAGGGTGGTCCTCCATAGAACTTATAAGACTCCCATACTCAAAGACATTTCACGTTTATGGTGACTTCCCA
GAAGACATAGCGACATGCAAATATTGCAGGGCGTCACACCCCCTGTCCCTTACAGCTCTCTTCCTGCCAGGGCGC
ACGCGCGCTGGGTGTTCCCGCGTAGTGACACTGGGCCCGCGATTCCTTGGAACGGGTTGATGACGTCAGCGTTCG
AATTAC
>Big_brown_bat_H1_Bidirectional_Promoter
(SEQ ID NO: 27)
GGGAAGCGAGCGTCACACGGCGGATATATAAGGCCCCCTTACCTGAAGGCCTTTTACGGTTAGGGTGACTTCCCA
CAACACTTAGCGACATGCAAATTTAGACGGGCGTGCCTCCCCGTCCCTGGGCAACTTCTCTCCTGGACACGCGCG
CTCGCGCTGAGTGTTCCCGCCTTTTGACTCCAGCCGAGCGAATCCTGGGAGAGGGCAGATGACGTCAACAGTCAG
GCTCC
>Black_flying-fox_H1_Bidirectional_Promoter
(SEQ ID NO: 28)
GAGAGAAAAAGCCTGCACGCAGAATATATAAGGATCCCATATCTGAAGACATTTTACGGTTACGGTGATTTCCCA
CAACACATAGCGACATGTAAATATAGTGGGGCATGCCTCTCCTGTCCCTGGGCAGCTTCTCGCCAGAACGCACGC
GCGGTGCGTGTTCCCGCCTTGTGACTAAGTTGGCGAGTCAGGGAGGAGATTGATGATGTCATCATCGTCAGCTCA
CCCGCTCC
>Black_snub-nosed_monkey_H1_Bidirectional_Promoter
(SEQ ID NO: 29)
GGGGAAGGGTGGTCCTACACAGAGCTTATAAGACTCCCATACTCAAAGACATTTCTCGTTTATGGTGACTTCCCA
GAAGCCATAGCGACATGCAAATATTGCAGGGCGTCACACCCCTGTCCCTTACAGCCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
CTTCC
>Bonobo_H1_Bidirectional_Promoter
(SEQ ID NO: 30)
GGGAAAGGGTGGTGCCACACAGAACTTATAAGACTCCCATATCCAAAGACATTTCACGTTTATGGTGATTTCCCA
GAACACATAGCGACATGCAAATATTGCAGGGCGCCACTCCCCTGTCCCTCACAGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Brush-tailed_rat_H1_Bidirectional_Promoter
(SEQ ID NO: 31)
GAAGGAAGTTAGTCACAAACGCAAATTATAAGAGGTCCAAAGCTCAGTGTACTCTATGGTTAGGGTGACTTCCCA
CAATACATAGCGATATGCAGATTTCTTCCCCAGTCTGGCCCGCTGGGCCCTCCCTAGAGCGCATGCGCTGCAAGT
CCACGGCGGAGCACCGGGCGGGCGATCCCGGAGCGGGTTGATGACGTCAGCGTTTGAACTCC
>Camel_H1_Bidirectional_Promoter
(SEQ ID NO: 32)
GAGAAAGGGTGGGCTCACGCCACCTTTATAAGGCTCCCAAACTTAAAGACATTTCTCGGTTATGGCGACTTCCCA
CAACACATAGCGACATGCAAATACTGCAGACCTGTGGCGCCGACCCGGTCCTGTGCAGCCATCTTTAAGGCTGGG
ACGCACGCGCGCTGCGTGTTCCCGCCCTGTGACTGCGCCGGCGATTACTGGGAGAGGATTGATGACGTCAACGTT
CGGGTTCC
>Cape_golden_mole_H1_Bidirectional_Promoter
(SEQ ID NO: 33)
GGGCTAACACTGTGTTGGTATTAGCTTATAAGAAACCCAAATATAAAGTCATTTAACGCTTAGTGTGACTTCCCA
TCATACAAAGCGACATGCAAATATCATGGGCCTTCCGGGAGGCGTGCCTTCCCGTCCTGCGTACTGGAGTTCTCT
CTGGGGCGCACGCGCGCTATGTGTTTCCCGCCTTGTGACTTAGGGCGGGCGATTCCTGAGATCCGAATGGTGACG
TCAACTTTCAGGCTCG
>Chinchilla_H1_Bidirectional_Promoter
(SEQ ID NO: 34)
GAAAGCCGAAGGTTTGGAGCGAAACTTATAAGAAGCCCAAATCTCACTATATTTTTAGGTCATGGCGACTTCCCA
CAAGCCACAGCGATATGTAGATATAGGAGCCCCTCCCAGTTCTGGTCCTTCCGCGTCTCACTAAAGCGCATGCGC
TGCAGGTTCGCGGCCTGCGACTGGGCCTGCAATTCCTGGGAGCGAGTTGATGACGTCAGCGTTTGAACTCC
>Chinese_hamster_H1_Bidirectional_Promoter
(SEQ ID NO: 35)
ACAGCCTGGTGAATGGCGGGCTTTATAAGGCTCCGGAGAGAAAGCGCTTTCTCAGTTATGGTGGTTTCCCACAAG
GCACAGCGCACACTTTATTTGCATGCGATCTAGCGCAGGCTCCCGCTCCAGACAAGAAGCCCGCGCTTTTCGGCT
GCTTATGATGACGTCGGGCCTCAAGCGCC
>Chinese_tree_shrew_H1_Bidirectional_Promoter
(SEQ ID NO: 36)
GGGGGAAGCTGGGTCCACTGAGTTCTTATAAGGTTTCCAGTCCTAGAGCGATTTTACCATTACGGTGATTTCCCA
GCATCCGTAGCTACATGCAAATAGCGCGGGGCGCGTCTCTCAGGTCCCTCCCCCGTGCCCTCTCACTGTACGTAC
CCGCGTCCTAGGGACGCCGCGCCCGGGGTTCCCGGACGTCAGCGTTCCGACGCA
>Consensus-1_H1_Bidirectional_Promoter
(SEQ ID NO: 37)
GGGGAAGGGTGGTCCCACACAGAACTTATAAGGCTCCCATACCTAAAGACATTTCACGGTTATGGTGACTTCCCA
CAAGACATAGCGACATGCAAATATTGCAGGGCGTCCCTCCCCTGTCCCTAGGCATCTTCTCGCCAGGGCGCACGC
GCGCTGCGTGTTCCCGCCTTGTGACACTGGGCCCGCGATTCCTGGGAGCGGGTTGATGACGTCAGCGTTCGAGCT
CC
>David's_myotis_H1_Bidirectional_Promoter
(SEQ ID NO: 38)
GAGAGGGGCTGTGCACACGGCGGATATATAAGGCCCCCTTATGAATAACCCTTTATAAGTTATGGTGATTTCCCA
CAACGCATAGCGACATGCAAATTCGATGGGCGTGCCTCCTCTGTCCCCAGGCAACTTCTCTCCTGGACGCGCGCT
CCTCGCTGCGTGTTCCCGCCTTTTGACTCCAGCCGAGCGAATCCTGGGAGAGGGCAGGTGACGTCAACAGTCAGG
CTCG
>Drill_H1_Bidirectional_Promoter
(SEQ ID NO: 39)
GGGGAAAGGTGGTCCCACACAGAACTTATAAGATTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGATGTTCCCGCGTAGTGACCCTGGGCCCGCGATTCCTTGGAGCGGGTTGATGACGTCAGCGTTCGA
ATTCC
>Gibbon_H1_Bidirectional_Promoter
(SEQ ID NO: 40)
GGGGAAAAGTAGTTTTTTTTAGACCTTATAAGATTCCCAAACCCAAAGACATTTCTCGTTTATGGTGACTTCCCA
GAAGACATAGCGACATGCAAATATTGCAGGGCGCCACTCCCCTGTCCCTCACAGCCATCTTCCTGCCAGGGCGCA
CGCGCGCTGGGTGTTCCCGCCTAGTGACACTCGGCCCGCGATTCCTTGGAGCGGGTTGATGACGTCAGCGTTCGA
ATTCC
>Goat_H1_Bidirectional_Promoter
(SEQ ID NO: 41)
GGCAAACAACGCGCGCAAACAGCATTTATAATGAGCTCATACCTAAAGCCACTTTACGATTACGGTGACTTCCCA
CAAGACATTGCGGCATGCAAATATTTTAGTGCGTCCCGCCCCTGGTAGTTCCACGCTAGGACGCACACGCACTAC
GGTTCCCGCCTTTAGACTGCGCTGGCGATTCCAGCGAGCGGACTGATGACGTCAGCGTTGGGGCTCC
>Golden_hamster_H1_Bidirectional_Promoter
(SEQ ID NO: 42)
GTGGCCCGGCGGCGGGCGAACTATATAAGCCTCCGCGGAGGAAGCGCTTTCTCGGTTAGGGTGGTTTCCCACAAG
CCTCAGCGCACAGCCTCTTTGCATACGCTCCCGCCGCCCCCGGGCTCCTCCCTCTCCGCACAAGAAGCCCGCGCA
TTTCGACTGCGGATGATGACGTCGGGCCTCGAGCGCC
>Golden_snub-nosed_monkey_H1_Bidirectional_Promoter
(SEQ ID NO: 43)
GGGGAAGGGTGGTCCTACACAGAGCTTATAAGACTCCCATACTCAAAGACATTTCTCGTTTATGGTGACTTCCCA
GAAGCCATAGCGACATGCAAATATTGCAGGGCGTCACACCCCTGTCCCTTACAGCCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Hedgehog_H1_Bidirectional_Promoter
(SEQ ID NO: 44)
GCCTAAACCGGCTCTTTCAACAGACTTATAAGGACCTCTTATCTTAGGACATTTTTTTCTTAGGGTAACTTCCCA
TGATGCACAGCGATATGTAAATATGGCGCCGCGAGTCTCTCCTAGGCGTCTCCCCAGGACGCAGGCGCACTGCTT
GTTCCCGCGTTAACATTGCTGATTCTGGGAGACTGCTGATGACGTCAGCGTCCAGTCTAC
>Killer_whale_H1_Bidirectional_Promoter
(SEQ ID NO: 45)
GCCGAAAACCAGGCTCAAGCCACATTTATAAGGCTCCCAAACCTAAATACATTTGTCGGTTATGGTGACTTCCCG
CAACACATTGCGACATGCAAATACTGCGGAGCGTTCCTCCCCTAGCAACTCCTCGCTGGGACGCACGCGCGCTAC
GTGCTCCCGCCTTTTGACCGAGCCGGCGATACTTGGGAGAGGGTTGATGACGTCAGCGTTCTGGCTCC
>Lesser_Egyptian_jerboa_H1_Bidirectional_Promoter
(SEQ ID NO: 46)
GGGCAGACCTTAACCAAGCGGAGGTTTATAAAGCGCCCACATTCAGTGACACTTCTCAGTCACGGTGACTTCCCA
CAAAACACAGCGCATGCAAATATTATGGCGGGAGGGGGGGTGCTCGCCTGGGCGCACGCGCGCTGTGGGTTCCCG
CGAGCGGGATGATGACGTCACTAAGTGAGC
>Manatee_H1_Bidirectional_Promoter
(SEQ ID NO: 47)
GAGCCAAACAGCTGTTGGTCACATTATATAAGAATCCCATATATAAAGACATTTTTGGCGTAGGGTGACTTCCCA
CAATACATAGCGACATGCAAATACCATGGTCCTCCAGGAGGCGTGCCTCCCCGTCCCCTTGGTCCGGTTCTTGCT
GGGGCGCACGCGCGCTGCGTGTTCCCGGTCTGTGACTCAGCTCGCGATTCCGGAGAGCGGATTGGTGAAGTCAAT
GTTCTGGGTCC
>Mas_night_monkey_H1_Bidirectional_Promoter
(SEQ ID NO: 48)
GGGGAAGGGTGGTCCTATACAGAACTTATAAGACTCCCATACCCAAAGACATTTCACGGTTATGGTGACTTCCCA
GAAGACACAGCGACATGCAAATATTGTAGGTCGTGCCTCGCTTGTCCCTCAGTAGTCTTCCTTTCAGAGCGCACG
CGCGCTGGGTGTCCCGCCAACTGACACTGGGCTCGCGATTCCTTGGAGCGGGTTGATGACGTCAGCGTTCGAATT
CC
>Microbat_H1_Bidirectional_Promoter
(SEQ ID NO: 49)
GGAGAAGGAGGCGTAGACGGCGGATATATAAGGCCCCCTTATGTGTAGTCCTTTTACGGTTAGGGTGACTTCCCA
CAACGCATAGCGACATGCAAATTTGACGGGCGTGCCTCCTCTGTCCCCGGGCAACTTCTCTCCTGGACGCGCGCT
CGCGCTGCGTGTTCCCGCCTTTTGACTCCAGCCGAGCGAATCCTGGGAGAGGGCAGGTGACGTCAACAGTCAGGC
TCG
>Opossum_H1_Bidirectional_Promoter
(SEQ ID NO: 50)
GGTGCGGGGCCTCAAAGAGAGCGATATATAACGCTCACAAAACCCGTGCTATTTCTTACAGAGGGTGATATCCCC
ATGATCCCCGGCGGTATGCAAATAGTAGTCGCGTCAGAGCAGAGCGCAGTCAGCCGCTCTCTCCTAGCGCGGGAA
ATCTATTTCTTCTTCAGTCTCGGTAACGAGCGCATGCGCATACTGTAGGTGACCTACGGTTTTGTCAGGAATCGG
TTGGGAGCACC
>Pacific_walrus_H1_Bidirectional_Promoter
(SEQ ID NO: 51)
GGGAAACGGTGGCCCCAAAGAGCATTTATAAAGCTCCCTCAACTAAATGCATTTATCAGTTATGGTGACTTCCCA
CAATACATCGCAACATGCAAACATCGCGGGGAGTACCTCCCCTGTCCCTACGTGTCTTCTCAGGACGCACGCACG
CGCGCTGTGTTCCCGCCCTGTGACTCTAGGCGGGCAATTCCTAGAAGACGCTTGCTGACGGGAACGTTCCGGCTC
C
>Pig-tailed_macaque_H1_Bidirectional_Promoter
(SEQ ID NO: 52)
GGGGAAAGCCGATCCCAGCCAGAACTTATAAGATTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Prairie_vole_H1_Bidirectional_Promoter
(SEQ ID NO: 53)
GGGAAGGCGGGGCGGCGGCACTAAAAGGCTCCGGAGCGGCCCAGACTTTACAGTTATGGTGGCTTCCCACGAGGC
GCAGCGCCACTCATTTGCATGGACCCGCCCCAGACGGGAAGCCCGCACCGCTCATTTGTGTGGCCCCGCCCCAGA
CGGGAAGCCCGCGCCACTCATTTGC
>Rhesus_H1_Bidirectional_Promoter
(SEQ ID NO: 54)
GGGGAAGGGTGGTCCCACACAGAACTTATAAGATTCCCATACTCAAAGACCTTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Ryukyu_mouse_H1_Bidirectional_Promoter
(SEQ ID NO: 55)
TGGAGGGTGGAGACCGGCCGCCACTATAAGGCTCGAAAGAGGAATAAATTTTACGTTTAGGGTGATTTCCCACAA
AGCACAGCGCGTAATTTGCATGTGCTCTATCCCAGGCTCCAGTGCCAGACAAGAAGCCCGCGCATCCGGGCAAGG
GATGATGACGTCGTCCTTCAAGAGCG
>Shrew_H1_Bidirectional_Promoter
(SEQ ID NO: 56)
GCGTAAGACGCGCCGCATCGCGTACTTATAAGGATCCCCTGGTCAACGATCTTTTACAGTTAGGGTGACTTCCCA
CAGTACACGGCGGTATTCAAATATGAAGGGCGTGTCTAGTCCGGGTCCTGGCTAGGCGCATGTGCAGTGCTGGTT
CCCGCCACTTCCGACGTCTACGTTTAGACTCC
>Shrew_mouse_H1_Bidirectional_Promoter
(SEQ ID NO: 57)
TGAAGGCTGTAGACCGGCCGCCACTATAAGGCTCGAAAGAGGAATAAGTTTTTCGCTTACGGTGACTTCCCACAA
AGCACAGCGCGTAATTTGCATGTACTCTATCCCAGGCTTCCTGTTCCAGACTAGAAGCCCGCGCATCCGGGCAAG
GGACGATGACATCATCCCCATCCCTCCAGCGCG
>Sifaka_H1_Bidirectional_Promoter
(SEQ ID NO: 58)
GAGGGAAAAGGGTTCTGCACAGAATTTATAAGGCTCCCAAATCTAAAAACATTTCACCATTATGGTGATTTCCCA
CAACACATAGCGACATGCAAATATCTCAGAGCGTACCTCCCCTGTCCTATACGGGCGTCAACTCGCCATGGCGCA
CGCGCGTTGTGTGTTTCCCGCCTGTGACTCTGGGCCCGCGATTCCTCCCAGCGGGTTGAGTACGTCAGCTCCGGT
GCTTC
>Sooty_mangabey_H1_Bidirectional_Promoter
(SEQ ID NO: 59)
GGGGAAAGGTGGTCCCACACCGAACTTATAAGACTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGCAGCGGGTTGGTGACGTCAGCGTTCGA
ATTCC
>Squirrel_monkey_H1_Bidirectional_Promoter
(SEQ ID NO: 60)
GGGGAAGGGTGGTCCTTCGCAGAACTTATAAGATTCCCAGTCCCGAGGACATTTCTAGATTATGGTGACTTCCCA
GAATACACAGCGACATGCAAATATTGCAGGTCGTGCCTCGCCTGTCCCTCACTGTCGTCTTCCTGCCAGGGCGCA
CGCGCGCTGGGTGTCCCGCCAACTGACACTGGGCTCGCGATTCCTTGGAGCGGGTTGATGACGTCAGCGTTCGAA
TTCC
>Star-nosed_mole_H1_Bidirectional_Promoter
(SEQ ID NO: 61)
GCGCAGAGACAAGCTTAGCTAGAATTTATAAGGCGCCCATACTTGCAGACATATATCGGTTAGGGTGACTTCCCA
CAAGCCATAGCGACATGCAAATAGAGAGGGCGGGCTTCCCCTGAGCTTAGGCGTCTTCTTACGAAGTCGCGAGCG
CGTCGCGCGCCTGTTCCCGCCCGGTCACTATTGGCCTGTCACTATTGTCATTCCGCCCTTCCCGGGCGGAGTCTG
GTGACTTTCGGTTCC
>Synthetic-1_H1_Bidirectional_Promoter
(SEQ ID NO: 62)
GCAGCGCAGCCCTCTCGCCGCTTATAAAGTGCCGCCCGCACGGCCCTTCTCGCTCACGGCGACTTCCCATAAAGC
ACAGCGCGTAATTTGCATGCGCTCTACCCCAGGCTCCTGTGCTAGACAAGAAGCCCGCGCATCCGGGCAAGGGAT
GATGACGTCAGATCTCC
>Synthetic-2_H1_Bidirectional_Promoter
(SEQ ID NO: 63)
GGGGAAAAGTAGTGCCGCTTATAAAGTGCCGCCCGCACGGCCCTTCTCGCTCACGGCGACTTCCCATAAAGCACA
GCGCGTAATTTGCATGCGCTCTACCCCAGGCTCCTGTGCTAGACAAGAAGCCCGCGCATCCCGGACGTCAGATCT
CC
>Tenrec_H1_Bidirectional_Promoter
(SEQ ID NO: 64)
AGGTTAAAGCCGCGTCGCCGCGCGCTTATAAGAATCCGGGAACTAACTACATTTCAAGGTCAGGGTGATTACCCA
CCCTGCATAGCGACATGCAAATAGCACGGAACGTCCAGGAGACGTGCCTCTAGGTCTTGGGGAGGGAGGAGTTCG
GCCCAGCGCGCACGCGCACTACGTGTTCCCGCCCGCTGTCTCGGGGGGGGAGATCCCGGGTAGGTGACGTCAGTC
CTCGGCTTC
>Tibetan_antelope_H1_Bidirectional_Promoter
(SEQ ID NO: 65)
GGCAAACGACTCCCGCAAACAGCATTTATAATGCGCTCATACATAAAGCCACTTTTCGGTTACGGTGACTTCCCA
CAAGACATTGCGACATGCAAATATTTTAGTGCATCCCGCCCCTGGTAGCTCCACGCTAGGACGCACACGCACTAC
GGTTCCCGCCTTTAGACTGCCGGGGCGATTCCAGGGAGCGGACTGATGACGTCAGCGTTCGGACTCC
>Tree_Shrew_H1_Bidirectional_Promoter
(SEQ ID NO: 66)
GGGGGAAGCTGGGTCCACTGAGTTCTTATAAGGTTTCCAGTCCTAGAGCGATTTTACCATTGCGGTGATTTCCCA
GCATCCGTAGCTACATGCAAATAGCGCGGGGCGCGTCTCTCAGGTCCCTCCCCCGTGCCCTCTCACTGTACGTAC
CCGCGTCCTAGGGACGCCGCGCCCGGGGTTCCCGGACGTCAGCGTTCCGACGCA
>Weddell_seal_H1_Bidirectional_Promoter
(SEQ ID NO: 67)
GGGGAAGAGTGGCCCCAGAGAGCATTTATAAGGCTCCCGCACCTAAATGCATTTATCAGTTATGGTGACTTCCCA
CAATACATAGCAACATGCAAATATAGCGGGGAGTACCTCCCCTGTCCCTACGTGTCTTCTCAGGACGCACGCACG
CGGGCTGTGTTCCCGCCCTGTGACTCTAAGCGGGCAATTCCTGGAAGAGGCTTGCTGACGGGGACGTTCAGGCTC
C
>White_rhinoceros_H1_Bidirectional_Promoter
(SEQ ID NO: 68)
GGAGCAAACATGCGCCAGGCAGCCTTTATAAGACTCACATATCTAAAGACATTTCACAGTTAGGGTGACTTCCCA
CAGGACACAGCGATATGCAAATATCGTGGAGCGTACCTCCCCAGTCTCCGGGCATCTTCTCGCCTACACGCACGC
GCGCCGCGTGTTCCCGCCCTGTGACGCTAGGTGGGCCTTTCATGGGAGAGGGTTGATGACGTCAACATTCGGACT
CC
>White-faced_sapajou_HI_Bidirectional_Promoter
(SEQ ID NO: 69)
GGGGAAGGGGTGGCCTACGCAGAACTTATAAGATTCCCACACCTAAAGACATTTAACGATTATGGTGACTTCCCA
GAATACACAGCGACATGCAAATATTGCAGGTCGTACCTCGCCTGTCCCCCACAGTCGTCTTCCTGCCAGGGCGCA
CGCGCGCTGGGTGTCCCGCCAACTGACAGTGGACTCGCGATTCCTTGGAGCGGGTTGATGACGTCAAAGTTCGAA
TGCC
>Alpaca_H1_Bidirectional_Promoter
(SEQ ID NO: 70)
GGGAAAGGGTGGGCTCACGCAGCCTTTATAAGACTCCCAAACTTAAAGACATTTCTCGGTTATGGCGACTTCCCA
CAAGACATAGCGACATGCAAATACTGCAGACCTGTGGCGCCGACCCGGTCCTGTGCAGCCATCTTTACGGCTGGG
ACGCACGCGCGCTGCGTGTTCCCGCCCTGTGACTGCGCCGGCGATTACTGGGAGAGGATTGATGACGTCAACGTT
CGGGTTCC
>Armadillo_H1_Bidirectional_Promoter
(SEQ ID NO: 71)
AAAGCGATAGTTTTTTAAACTGGACTTATAAGGCACCCATATCTACGTATATTTCATGGTTAGGGTGATTTCCCA
CAACACATAGCGAAATGCAAATATGTGGAGCGGGCGCTGAGGCGTGGTCGGGCGCAAGCGCGCTGCGACTTCCCG
CCTTTCGGCCCTAGGCCCCAGATTCCTGGGAGCTGGATGATGACGTTGACGTTCGGATACC
>Baboon_H1_Bidirectional_Promoter
(SEQ ID NO: 72)
GGGGAAAGGTGGTACCATACAGAACTTATAAGATTCCCATACTCAAAGACATTTCACGATTATGGTGACTTCCCA
GAAGACACAGCGACATGCAAATATTGTAGGGCGTCACACCCCCTGTCCCTCACAGTCATCTTCCTGCCAGGGCGC
ACGCGCGCTGGGTGTTCCCGCGTAGTGACACTGGGCCCGCGATTCCTTGGAGCGGGTTGATGACGTCAGCGTTCG
AATTCC
>Bottlenose_dolphin_H1_Bidirectional_Promoter
(SEQ ID NO: 73)
GCCGAAAACCAGGCTCAAGCCACATTTATAAGGCTCCCAAATCTAAGTACATTTGTCGGTTATGGTGACTTCCCG
CACCACATTGCGACATGCAAATACTGCGGAGCGTCCCTCCCCTGGCAACTCCTCGCTGGGACGCACGCGCGCTAC
GTGCTCCCGCCTTTTGACTGCGCCGGCGATACTTGGGAGAGGGTTGATGACGTCAGCGTTCTGGCTCC
>Bushbaby_H1_Bidirectional_Promoter
(SEQ ID NO: 74)
GCCTAAAAGGGCGCTTGCACAGAATTTATAAGGTTCCCAAACAGAGACACATTTCATTATTATGGTGACTTCCCA
CAATGCACAGCGCCATGCAAATATGCTAGGACCTGCCTCCCCACACCCGCTACCTTAAGGTCGTCAACTAACCAG
TGCGCGCGCGCACTGCGCGTTTCCCGCCGGTGACTCAATGCCCGCGTTTGGTGGGAGCTAGTTGGTGACCTCAGT
TCTGGAGGCTC
>Cat_H1_Bidirectional_Promoter
(SEQ ID NO: 75)
GGGAAAGGGTGGCCCCGCCGAGCATTTATAAGACTCCCATACCTAAAGACATTTCTCAGTTATGGTGATTTCCCA
CAACACACAGCAACATGCAAATATCGAGGGGTGTACCGCCCCTGTCCTTAGACGTCTTCTCTCCAGGACGCACGC
GCGCTGTATTCCCGCCTTGTGACTCTAGGCGGGCGATTCCTGGGAGAGGGTTGATGACGTCCAAGTTCTGGCTTC
>Chimp_H1_Bidirectional_Promoter
(SEQ ID NO: 76)
GGGAAAGGGTGGTGCCACACAGAACTTATAAGACTCCCATATGCAAAGACATTTCTCGTTTATGGTGATTTCCCA
GAACACATAGCGACATGCAAATATTGCAGGGCGCCACTCCCCTGTCCCTCACTGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Cow_H1_Bidirectional_Promoter
(SEQ ID NO: 77)
GGCAAACACCGCACGCAAATAGCACTTATAATGTGCTCATACCTAGAGCCACTTTTCGGTTACGGTGACTTCTCA
AAAAGACAGTGGAACATGCAAATATTACAGTGCGTCCCGCCCCTGGTAGGTCTACGCTAGGACGCACGCGCACTA
CGGTTCCCGCCTATAGACTGCGCTGGCGATTCCTGGGAGCGGACTGATGACGTCAGCGTTCGGGATCC
>Crab-eating_macaque_H1_Bidirectional_Promoter
(SEQ ID NO: 78)
GGGGAAGGGTGGTCCCACACAGAACTTATAAGATTCCCATACTCAAAGACATTTCTCGTTTATGGTGACTTCCCA
GAAGACACAGCGACATGCAAATATTGTAGGGCGTCACACCCCTGTCCCTCACAGTCATCTTCCTGCCAGGGCGCA
CGCGCGCTGGGTGTTCCCGCGTAGTGACACTGGGCCCGCGATTCCTTGGAGCGGGTTGATGACGTCAGCGTTCGA
ATTCC
>Dog_H1_Bidirectional_Promoter
(SEQ ID NO: 79)
GCAGCGCAGCCCTCTCGCCGCTTATAAAGTGCCGCCCGCACGGCCCTTCTCGCTCACGGCGACTTCCCATAACAC
ACAGCAGCATGCAAATACCGCGGGGAGCCCCGCCCCGCCCCGGCCCCCGCACCGCCTCGGGACGCATGCGCCGGC
TCTCCGTTCCCGCCTTGGGCCGGCGGCGGGGGGGGGGGGAGCGGGCGGGAGCGGCTCCGGCGAGCGGGCGCC
>Elephant_H1_Bidirectional_Promoter
(SEQ ID NO: 80)
GGGATAGGAACAAATTCGTCAGGATTTATAAGACTCTCAGAGCTGTAGACATTTCACAGTTAGGGCGATGTCCCA
CAATACATAGCAACATGCAAATACATGAGCCTTCTAGGAGGCCAGCCTCCCCGTCCGCGTGGTCATCTTCTCGCT
AGGGCGCACGCCCGCTGCGTGTTCCCGCTCTGTGACCAGGCAGGCGATTCCTGAGAACCGCTTGGTGACGTCAGT
GTTCTGGCTCC
>European_Hedgehog_H1_Bidirectional_Promoter
(SEQ ID NO: 81)
GCCTAAACCGGCTCTTTCGACAGACTTATAAGGACCTCTTATCTTAGGACATTTTTTTGTTAGGGTAACTTCCCA
CGATGCATAGCGATATGTAAATATGGCGCCGCGAGTCTCTCCTAGGCGTCTCCCCAGGACGCAGGCGCACTGCTT
GTTCCCGCGTTAACATTGCTGATTCTGGGAGACTGCTGATGACGTCAGCGTCCAGTCTAC
>Ferret_H1_Bidirectional_Promoter
(SEQ ID NO: 82)
GGGAAAGGGTGGACCCACCGAGCATTTATAAGGCTCCCGCATCTAAAGACATTTTACAGTTATGGTGACTTCCCA
CAACGCGTAGCAACATGCAAATATCGTGGAGAGTACCGCCCCTGTCCCCACGCGTCTTCTCAGCACGCACGCACG
CGCGCTGTGTTCCCGCCCTGTGACTCCAGGCGGGTATTTCCAGGGGCGGGTTTGCTGACAGGAACGTTCAGGCTT
C
>Gorilla_H1_Bidirectional_Promoter
(SEQ ID NO: 83)
GGGAAAGGGTGGTCCCACACAGAACTTATAAGACTCCCATATCCAAAGACATTTCACGGTTATGGTGATTTCCCA
GAACACATAGCGACATGTAAATATTGCAGGGCGCCACTCCCCAGTCCCTCACAGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Green_monkey_H1_Bidirectional_Promoter
(SEQ ID NO: 84)
GGGGAAGGGTGGTCCCTTACAGAACTTATAAGATTCCCAAACTCAAAGACATTTCACGTTTATGGTGACTTCCCA
GAAGACATAGCGACATGCAAATATTGCAGGGCGTCACACCCCTCTCCCTCACAGTCATCTTCCTGCCAGGGCGCA
CGCGCGCTGGGTGTTCTCGCGTAGTGACACTGGGCCCGCGATTCCTTGGAGCGGGTTGATGACGTCAGCGTTCGA
ATTCC
>Guinea_pig_H1_Bidirectional_Promoter
(SEQ ID NO: 85)
GAGAAAGAAAGGCTCAAACCTAGCCTTATAAGGCTCCCAAATGTCGGTATATTTTTTGGTTATGGTGACTTCCCA
CAATGCATAGCGATATGTAGATATAGGAGTACCTCCCACTTCTGGTCCGTCAGCTCTTTTCTAGGACGCGCGCGC
TGCAGGTTTCCAGCCTGTGATTGGGCCAGCAATTCCGGGAATGAATTGATGACGTCAGCGTTTGAATTCC
>Horse_H1_Bidirectional_Promoter
(SEQ ID NO: 86)
GGGGGAAAACAGCCCATGGCTGCATTTATAAGACTCACAGATCTAAAGCCATTTCACGAATAGGGTGACTTCCCA
CAATACACAGCGACATGCAAACATAGCGGGGCGTGCCTTTCCTGTACCTGGGCATCTCTCCTGGACGCACGCGCG
CCGGGTGTTCCCGCGCTGTGACTCTAGGCAAGCGCTTCCTGGGAGAGAGTTGATGACGGCAGCATTCGGGCTCC
>Human_H1_Bidirectional_Promoter
(SEQ ID NO: 87)
GGGAAAAAGTGGTCTCATACAGAACTTATAAGATTCCCAAATCCAAAGACATTTCACGTTTATGGTGATTTCCCA
GAACACATAGCGACATGCAAATATTGCAGGGCGCCACTCCCCTGTCCCTCACAGCCATCTTCCTGCCAGGGCGCA
CGCGCGCTGGGTGTTCCCGCCTAGTGACACTGGGCCCGCGATTCCTTGGAGCGGGTTGATGACGTCAGCGTTCGA
ATTCC
>Kangaroo_Rat_Bidirectional_Promoter
(SEQ ID NO: 88)
AGGAAAGACTTCGCTGAGGCAGACTTTATAAGGCTCCCGCGCAGAAAGAAACTTTATAGTTATGGTGATTTCCCA
CAAGCCACTGCGTCATGCAAATAAAGCAGGGTACGGCTTCCATGTACCTTAAGGTTTTTTTCTAGGCCGCGTACG
CTCTGCGTATTCAGCCACGTGACCCTGAGCCAGTGGTTGTTGGGAGCACGTTGTGGACCTCTGCGTTTGGATTCC
>Large_flying_fox_H1_Bidirectional_Promoter
(SEQ ID NO: 89)
GCGAGAAAAATTCTTCACGCAGAATATATAAGGATCCCATATCTGAAGACATTTTACGATTACGGCGATTTCCCA
CAACACATAGCGACATGTAAATGTAGTGGGGCATGCCTCCCCTGTCCCTGGGCAGCTTCTCGCCAGAACGCACGC
GCGGTGCGTGTTCCCGCCTTGTGACTAAGTTGGCGAGTCAGGGAGGAGATTGATGATGTCATCATCGTCAGCTCA
CCCGCTCC
>Little_Brown_Bat_H1_Bidirectional_Promoter
(SEQ ID NO: 90)
GGGAGAAGGAGGCGTAGAGGATATATAAGGCCCCCTTATGTGTAGTCCTTTTACGGTTAGGGTGACTTCCCACAA
CGCATAGCGACATGCAAATTTGACGGGCGTGCCTCCTCTGTCCCTGCGGGCAACTTCTCTCCTGGACGCGCGCGC
GCTGCGTGTTCCCGCCTTTTGACTCCAGCCGAGCGAATCCTGGGAGAGGGCAGGTGACGTCAACAGTCAGGCTCG
>Marmoset_H1_Bidirectional_Promoter
(SEQ ID NO: 91)
GAGGAAAAGTAGTCCCACAGACAACTTATAAGATTCCCATACCCTAAGACATTTCACGATTATGGTGACTTCCCA
GAAGACACAGCGACATGCAAATATTGCAGGTCGTGTTTCGCCTGTCCCTCACAGTCGTCTTCCTGCCAGGGCGCA
CGCGCGCTGGGTTTCCCGCCAACTGACGCTGGGCTCGCGATTCCTTGGAGCGGGTTGATGACGTCAGCGTTTGAA
TTCC
>Mouse_H1-1_Bidirectional_Promoter
(SEQ ID NO: 92)
TTCAGGATGTAGACCGGCCGCCACTATAAGGCTCGAAAGAGGAATAAATTTTTCGTTTAGGGTGATTTCCCACAA
AGCACAGCGCGTAATTTGCATGCGCTCTACCCCAGGCTCCTGTGCTAGACAAGAAGCCCGCGCATCCGGGCAAGG
GATGATGACGTCGTCCTTCAAGAGCG
>Mouse_H1-2_Bidirectional_Promoter
(SEQ ID NO: 93)
TTCAGGATGTAGACCGGCCGCCACTATAAGGCTCGAAAGAGGAATAAATTTTTCGTTTAGGGTGATTTCCCACAA
AGCACAGCGCGTAATTGCATGCGCTCTACCCCAGGCTCCTGTGCTAGACAAGAAGCCCGCGCATCCGGGCAAGGG
ATGATGACGTCGTCCTTCAAGAGCG
>Northern_Treeshrew_H1_Bidirectional_Promoter
(SEQ ID NO: 94)
GGGGGAAGCTGGGTCCACTGAGTTCTTATAAGGTTTCCAGTCCTAGAGCGATTTTACCATTGCGGTGATTTCCCA
GCATCCGTAGCTACATGCAAATAGCGCGGGGCGCGTCTCTCAGGTCCCTCCCCGCCCTCTCACTGTACGTACCCG
CGTCCTAGGGACGCCGCGCCCGGGGTTCCCGGACGTCAGCGTTCCGACGCA
>Orangutan_H1_Bidirectional_Promoter
(SEQ ID NO: 95)
GAGAAAGGGTGGTCCCGTCCAGAACTTATAAGATTCCCATACCCAAAGACATTTCACGTTTATGGTGACTTCCCA
GAATGCATAGCGACATGCAAATATTGCAGGGCGTCACTCCCCTGTCCCTCACAGCCATCTTCCTGCCAGGGCGCC
CGCGCGCTGGTGTTCCCGCCTAGTGACACTGGGCCCACGATTCCTTGGAGCGGGTTGATGACGTCAGCGCTCGTA
TTCC
>Panda_H1_Bidirectional_Promoter
(SEQ ID NO: 96)
AGGGAAAGCCGCGCCTGGGGCGGATTTATAAGGCTTCCATATCTAAAGGCATTTCACAGTCATGGTGACTTCCCA
CAATACATAGCAACATGCAAATATCGCGGGGAGAACCTCCCCTGTCCCTTGTACGCGGCTTCTAAAGACGCACGC
ACGCGCTCTGTGTTCCCGCCCTGTGACTCTAGGCGGGCAATTCCTGGGACAGTGTTCTGACGGGAACGTTCAGGC
TCC
>Pig_H1_Bidirectional_Promoter
(SEQ ID NO: 97)
GGAAAACTGCTTCTGTGAGCACTTATAAAACTCCCATAAGTAGAGAGATTTCATAGTTATGGTGATTTCCCATAA
GACATTGCGACATGCAAATATTGTGGCGCGTTCGTCCCCGTCCGGTGCAGGCAGCTTCGCTCCAGGACGCACGCG
CAATACATGTTCCCGCCTTGAGACTGCGCCGGCAGATTCCTAGGAAGTGGTTGATGACGTCGATGTTAGGGATCC
>Pika_H1_Bidirectional_Promoter
(SEQ ID NO: 98)
GGGGGAAGCTGGGCTCGATCAGCCTTTATAAAGCTCCAAAAACTCAAGACATTTTTCCGTTACGGTGGCTTCCCA
CAGTACACAGCGACATGCAAATAGGCGGACCGCTTCCCGCTCCGGCGCAGGCGCGCGGGCGCTGTCTCCCCTGGA
CGCGCGCTCGCGGTTCCCGGGAGCTGGCTGATGACGTTCGGTCTCC
>Rabbit_H1_Bidirectional_Promoter
(SEQ ID NO: 99)
GGGGAGAGGTGGATCCGAACAGACTTTATAAAGCTCCGAAAGCCCAAGGCATCTTTCCCTTACGGTAGCTTCCCA
CAAGACATAGCGACATGCAAATTTCAGACGCGCTTCTCGCCACAGCGCAAGCGCGCTGTGTGCTGACGCGGGAAC
GGGCCAGGGCGCGGTTCCCGGGAGCGGGTTGATGACGTTAGATCTCC
>Rat_H1_Bidirectional_Promoter
(SEQ ID NO: 100)
AGGAGTGTGAAGACCTGCCGCCATAATAAGACTCCAAAAGACAGTGAATTTAACACTTACGGTGACTTCCCACAA
AGCACAGCGTGTAATTTGCATGCGCTCTAGCCCAGGCTCCAGCTCCGGACCAGAAGCCCGCGCATCCCGGCAAAG
GGTGATGACGTCGTCCTTCAAGCGCT
>Rock_Hyax_Bidirectional_Promoter
(SEQ ID NO: 101)
AGGGTAAATCGGCGCTGCTCAGCATTTAAAAGAATCCCAAATGTGTCGCCATTTTACGCTTAGGGTGATATCCCA
CAAGACACAGCGACATGCAAATATCGTGAGTCTCTGTTTCCCTGTCCACGAGGGCGTCCTCTCGCTGGGGCGCAC
GCGCGGTGTGTGTGCCCCCGTTGTGTGTTCCCGCGATTCCAAAGAACTGGTTGATAACGTTAGACTTCCGGCTGC
>Sheep_H1_Bidirectional_Promoter
(SEQ ID NO: 102)
GGCGAACAATGCGCGCAAACAGCATTTATAATGAGCTCATACCTAAAGCCACTTTACGGTTACGGTGACTTCCCA
CAAGACATTGCGGCATGCAAATATTTTAGTGCGTCCCGCCCCTGGTAGTTCCACGCTAGGACGCACACGCACTAC
GGTTCCCGCCTTTAGACTGCGCTGGCGATTCCAGGAGCGGACTGATGACGTCAGCGTTGGGGCTCC
>Squirrel_H1_Bidirectional_Promoter
(SEQ ID NO: 103)
GAAAGGGACTCCGCACAAGCAGAGTTTATAAGGCTCCCATCTGTACAGCCATTTCTCGGTCATGGTAACTACCCA
CAACACACAGCGATATGCAAATATAGCAGAGCGTGTCTTCCCGCGCGCGCCTGGTCGTCTCGGCGCCGGCGCCGG
AACTGTGACAGAGACCCTGCGATTCCTGGGAGCTGGCTGATGACATCAGTGTCTAACCTCC
>Tarsier_H1_Bidirectional_Promoter
(SEQ ID NO: 104)
GCGAGAGGGTGGGTCCACACAGAGCTTATAAGGCTTCACAAGTAAAGATATTTCACGGTGACGGTGACTTCCCAC
AATACACTGCGACATGCAAATATAGCCGGGCGTGCCTCCCCGATCCCGGAAGAGCGACTCCTAGCCAGTGCGCAC
GCGCGCTGCGTGTTCGCGTCCTAGGTCGCTGGGCCCGCGGTTCCTGGGAGCGGGTGGTGACGTCAGCGGCCCAGC
TTC
>Two-Toed_Sloth_H1_Bidirectional_Promoter
(SEQ ID NO: 105)
AGAAAAAAATAGTTTATGCTGGATTTATAAGATTCCCAAATCTAAAGCCATTTCACAGTTACGGTGATTCCCCAC
TACACACGGCGATATGCAAATATAGCGGAAGTGTTCCTGAGGCGTGGTAAAGCGCGCGCGCGCTGAGAGTTCCCG
CCCTGTGGTGCTGGGCTGGAGATGCCTGAGAACTGGCTGATGACGGCAACGTTCGGGCTCC
>White_cheeked_gibbon_H1_Bidirectional_Promoter
(SEQ ID NO: 106)
GGGGAAAAGTAGTAGACCTTATAAGATTCCCAAACCCAAAGACATTTCTCGTTTATGGTGACTTCCCAGAAGACA
TAGCGACATGCAAATATTGCAGGGCGCCACTCCCCTGTCCCTCACAGCCATCTTCCTGCCAGGGCGCACGCGCGC
TGGGTGTTCCCGCCTAGTGACACTCGGCCCGCGATTCCTTGGAGCGGGTTGATGACGTCAGCGTTCGAATTCC
>GAR1-1_Bidirectional_Promoter_Homo_sapiens
(SEQ ID NO: 107)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCAGCGTCAG
>GAR1-2_Bidirectional_Promoter_Homo_sapiens
(SEQ ID NO: 108)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCAGCGTCAGGCAAGTTGGCCTCTC
TGTTGTAAATTAGTGGTTAAGGTTATCTATTATTGCCACTTTTCCAGCGCTAAAGGCTGTTTTGGAACCAGTGTT
GCTTGTTCCGCGGGTGATTGGCTTTTTTTTTTGGCAAACCAGTTATTCAAGTTTCTGGTCTTTAAAAAACTCTGT
GGCGGTACGGTAACCGAGGAGGTTCCAGCGCGGCGGAAGTACCCCGCGGGTGGGTGTGTGCGCAAGGCCAGGGCC
AGAGGGGCACGTGGCGCCG
>macaca_mulatta/1-143_Gar-1
(SEQ ID NO: 109)
CCCACCCCGCCTCTGGAAGAGAAGGCGGATCCCTAACGCCAGCTATCTCCAAGAGCAACATTGCCGCAGCACTTC
CGGGACGTCGTGCTGCGAAGGACGCAGCTATTATACGTCACTTCCACGGCGCGGCGTTAG
>ancestral_sequences9/1-143_Gar-1
(SEQ ID NO: 110)
CCTACCCCGCCTCTGGAAGAGAAGGCGGATCCCTAACGCCAGCTATCTCCAAGAGCAACATTGCCGCAGCACTTC
CGGGACGTCGTGCTGCGAAGGACGCAGCTATTATACGTCACTTCCACGGCGCGGCGTTAG
>papio_anubis/1-143_Gar-1
(SEQ ID NO: 111)
CCTACCCAGCCTCCGGAAGAGAAGGCGGATCCCTAACGCCAGCTATCTCCAAGAGCAACATTGCCGCACCACTTC
CGGGACGTCGTGCTGCGAAGGACGCAGTTATTATACGTCACTTCCACGGCGCGGCGTTAG
>ancestral_sequences10/1-143_Gar-1
(SEQ ID NO: 112)
CCTACCCAGCCTCCGGAAGAGAAGGCGGATCCCTAACGCCAGCTATCTCCAAGAGCAACATTGCCGCACCACTTC
CGGGACGTCGTGCTGCGAAGGACGCAGCTATTATACGTCACTTCCACGGCGCGGCGTTAG
>ancestral_sequences11/1-143_Gar-1
(SEQ ID NO: 113)
CCTGCCCCGCCTCTGGGAGAGAAGGCGGATCCCTAACGCCAGCTATCTCCAAGAGCAACATTGCCGCAGCACTTC
CGGGACGTCGTGCTGGGACGCCGCTATTATACGTCACTTCCACGGCTCCGCGTTAG
>callithrix_jacchus/1-143_Gar-1
(SEQ ID NO: 114)
CCCGCCCCGCCCCCGGTAGAGAGGGCGGATCTCTAACGCCAACTATCTCCAAGAGCAACATTGCCGCAGCACTTC
CGGGATGTCGTGCTGCGAAGGACGCCGCTATTGTACGTCACTTCCGCTTCTCCACTCTAG
>pan_paniscus/1-191_Gar-1
(SEQ ID NO: 115)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACAGCTCAGCGTCAG
>pan_troglodytes/1-191_Gar-1
(SEQ ID NO: 116)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCCGCGTCAG
>pongo_abelii/1-191_Gar-1
(SEQ ID NO: 117)
CCTGCCCCGCCTCTGGGAGAGAAGGCGGATCCCTAACGCCAGCTATCTCCAAGAGCAACGTTGCCACAGCACTTC
CGGGACGTCGTGCTGCAAAAGACGCCGCTGTTATACGTCACTTCCACGGCTCAGCGTTAG
>nomascus_leucogenys/1-191_Gar-1
(SEQ ID NO: 118)
CCTGCCCCGCCTCTGGGAGAGAAGGCGGATCCCTAACTCCAGCTATCTCCAAGAGCAACATTGCCGCAGCACTTC
CGGGACGTAGTGCTGCAAAGGACGCCGCTGTTATACGTCACTTCCACGTCTCAGCGTTAG
>chlorocebus_sabaeus/1-191_Gar-1
(SEQ ID NO: 119)
CCTACCCCACCTCTGGAAGGGAAGGCGGATCCCTAACGCCAGCTATCTCCAAGAGCAACATTGCCGCAGCACTTC
CGGGACGTCGTGCTGGGACGCAGCTATTATACGTCACTTCCACGGCGCCGCGTTAG
>macaca_nemestrina/1-143_Gar-1
(SEQ ID NO: 110)
CCCACCCCGCCTCTGGAAGAGAAGGCGGATCCCTAACGCCAGCTATCTCCAAGAGCAACATTGCCGCAGCACTTC
CGGGACGTCGTGCTGCGAAGGACGCAGATATTATACGTCACTTCCACGGCGCGGCGTTAG
>colobus_angolensis_palliatus/1-143_Gar-1
(SEQ ID NO: 111)
CCTGCCCCGCCTCTGGGAGAGAAGGCGGATCCCTAACGCCAGCTATCTCCAAGAGCGACATTGCCTCAGCACTTC
CGGGACGTCGTACTGCAAAGGACGCAGTTATTATACGTCACTTCCACGGCGCCGCGTTAG
>piliocolobus_tephrosceles/1-143_Gar-1
(SEQ ID NO: 112)
CCTGCTCCGCCTCTGGGAGAGAAGGCGGATCCTTAACGCCAGCTATCTCCTAGAGCAACATTGCCTCAGCACTTC
CGGGACGTCGAGCTGCAAAGGACGCAGTTATTATACGTCACTTCCAGGGCGCCGCGTTAG
>rhinopithecus_bieti/1-143_Gar-1
(SEQ ID NO: 113)
CCTGCCCCGCCTCTGGGAGAGAAGGCGGATCCCTAACGCCAGCTATCTCCAAGAGCGACATTGCCTCAGCACTTC
CGGGACGTAGTGCTGCAAAGGACGCAGTTATTATACGTCACTTCCACGGCGCCGCGTTAG
>aotus_nancymaae/1-143_Gar-1
(SEQ ID NO: 114)
CCCGCCCCGCCCCTGGGACAGAGGGCGGATCTCTAACGCCAACTATCTCCAAGAGCAACATTGCCGCAGCACTTC
CGGGACGTCGTGCTGCAAAGGACGCCGCTATTATACGTCACTTCCGCGGCTCCAG
>cebus_capucinus/1-143_Gar-1
(SEQ ID NO: 115)
CCCGCCCCGCCCCTGGGAGAGAGGGCGGATCTCTAACGCCAACTGTCTCCAAGAGCAACATTGCCGCAGCACTTC
CGGGACGTCGTCCTGCAAAGGACGCCGCTATTATACGTCACTTCTGCTGCTCACTGTAG
>saimiri_boliviensis_boliviensis/1-143_Gar-1
(SEQ ID NO: 116)
CCCGCCCCGCCCCTGGGAGAGAGGGCGGATCTCTAACGCCAACTATCTCCAAGAGCAACATTTCAGCAGCACTTC
CAGGACGTCGCCCTGCAAAGGACGCCGCTATTATACGTCACTTCCGCTGCTCCACTCTGG
>carlito_syrichta/1-143_Gar-1
(SEQ ID NO: 117)
CCTGCCCCGCCTCTAGAGAAGGGGACGGATTCGTAATGCCCGGCAATCGCGCAGCCGCATTTCCGGGACGTCACG
AGGAAAGGGCGCCGAATTGTATGTCATTTCCGCTTTTCATGGCTGG
>otolemur_garnettii/1-143_Gar-1
(SEQ ID NO: 118)
CTCGGCCAGTCTCAGGCAGAAAGGGCGGAAACCGGACCCCAGCGCAATGTCACGGCAGCACTTCCGGTATGCTCC
GTTGCAAAAGACGCTGCTATTGTACGTCACTTCCGCCACCCGGCTGG
>prolemur_simus/1-143_Gar-1
(SEQ ID NO: 119)
CCCGCCCCGCCTCTCGGAGACGGGGCGCGTCCCTCCCGCCGCCGTCTCCCGGGGCAACATGGCGGCAGCACTTCC
GGGGCGCCGGTGGCGAAAGGCGCCGCTATTATACGTCACTTCCGCCGCCCGGCGCGAG
>propithecus_coquereli/1-143_Gar-1
(SEQ ID NO: 120)
CTGGCCCAGCCTCTTATGGCGGGGGCGGACCCCTTACGCCAGCTATCGCCCAGGGCAATATGGCGACATCACTTC
CGGTATGTCAGGTTGTGAAAGGCGCCGCTATTGTACGTCACTTCCGCTGCCCAGCGCGGG
>castor_canadensis/1-143_Gar-1
(SEQ ID NO: 121)
CACAACTCGCCTCTGAGAGAGGAGGCGGATCCCTAACGCCTGCTATCTCCAAGGGCAACACTGCGGCATACTTCC
GGAACGTCAGCTCGATGGGACGCGGTTATTTTACGTCACGTCCGCTACTCTCACTCGG
>calJac3_Gar-1
(SEQ ID NO: 122)
CCCGCCCCGCCCCCGGTAGAGAGGGCGGATCTCTAACGCCAACTATCTCCAAGAGCAACATTGCCGCAGCACTTC
CGGGATGTCGTGCTGCGAAGGACGCCGCTATTGTACGTCACTTCCGCTGCTCCACTCTAG
>otoGar3_Gar-1
(SEQ ID NO: 123)
CTCGGCGTCAGTCTCAGGCAGAAAGGGCGGAAACCGGACCCCAGCGCAATGTCACGGCAGCACTTCCGGTTATGC
TCCGTTGCAAAAGACGCTGCTATTGTACGTCACTTCCGCCACCCGGCTGG
>speTri2_Gar-1
(SEQ ID NO: 124)
ACGCCCGACGGGAGAGGAGGCGGGTCCCTAACTCCGCTATCTCCTAGGGCAACTCGACGGCAATACTTCCGGTAA
CGTCCTGACGTAATGGATGCCGTTTCGCTTTACTTCCGCTTTCTCTTG
>micOch1_Gar-1
(SEQ ID NO: 125)
ACGCCCCGCTGTCTCCAAGGGCAACGAGAGACCTCACTTCCTGAAACGTCTCGTACAGAGGGCGCTGCTATTCTA
TGTCACTTCCGCTCCCCGGG
>criGril_Gar-1
(SEQ ID NO: 126)
AAGCCTCACTATAGGACGGAAGGATCCAGACTCCCGCTGTCTCCAAGGGCAACGCGCTACCACACTTCCGGAAAC
GTCGCGTACGGAGGGCACTGCTATTTTGCGTCACTTCCGCTACCCCGGC
>mesAurl_Gar-1
(SEQ ID NO: 127)
ACGCCTCACTCTAGAACGGAAGACTCCAGACGCCCGCCGTCTCCAAGGGCAACGCGCGACCACACTTCCGGAAAC
GGCGCGTACGGAGGGCGCTTCTATTTTGCGTCACTTCCTCTCCTCCAGG
>mm10_Gar-1
(SEQ ID NO: 128)
ACGCCTCACTGTAGCACGGAAGGACTCAAACAACTCCGTTTCCAAGGGCAACGCGCCGCCACACTTCCGGAAACG
TCGCGTACGGAGGGCGCTGCGATTTTGCGTCACTTCCGCCACCTCTAG
>microcebus_murinus/1-191_Gar-1
(SEQ ID NO: 129)
GCGGCGCCAGCCTCTGGGAGAGGGGGCGGACCCTTACGCCAGCTGTCTCCAAGGGCAATATAGCGGCAGCACTTC
CGGTAGCGACAGGTTGTGAAAGACGCCGCTGTTGTACGTCACTTCCGCTGCCCAGAGCGAG
>cavia_porcellus/1-191_Gar-1
(SEQ ID NO: 130)
CGAGTTGCTTCGGGCCTACTAACATCATGCGGCGTTTCTGGAAGAGGAGCCCGCTTCCGGACGCCCGCCGTCTCC
AGGGGCAACACTTCCGTGAACGTCATGTGTAAGGGACGGGTTACGTCACTTCCTGTGCTCCTTGGCT
>marmota_marmota_marmota/1-191_Gar-1
(SEQ ID NO: 131)
CGCCCGACTTCTGGCAAGAGGAGGCGGGTCCCTAACTCCGCTATCTCCTAGGGCAACACGACGGCAATACTTCCG
GTAACGTCCTGACGTAATGGTTGCCGTTTCGCTTTACTTCCGCTTTCTCTTGCTAA
>sciurus_vulgaris/1-191_Gar-1
(SEQ ID NO: 132)
CGCCCAGCCTCCGGGAAGAGGAAGCAGCTCCCGAATACCGGCTATCTCCAAGGGCAACACCACTGCAATGCTTCC
GGAAACGTCATGGCGTAATGGACGCCGTTACAACTTCACTTCCGCTTCTCTCGCTAC
>mus_caroli/1-191_Gar-1
(SEQ ID NO: 133)
CACGCCTCAACAGCTGTTAGCACGGAAGGACCCAAACAACCCCGTCTCCAAGGGCAATGCGCCGCCACACTTCCG
GAAACGTCGCGTACGGAGGGCGCTGCGATTTTGCGTCACTTCCGCCACCTCTAGCG
>mus_musculus/1-191_Gar-1
(SEQ ID NO: 134)
CACGCCTCACCAGCTGTTAGCACGGAAGGACTCAAACAACTCCGTTTCCAAGGGCAACGCGCCGCCACACTTCCG
GAAACGTCGCGTACGGAGGGCGCTGCGATTTTGCGTCACTTCCGCCACCTCTAGCG
>mus_spretus/1-191_Gar-1
(SEQ ID NO: 135)
CACGCCTCACCAGCTGTTAGCACGGAAGGACTCAAACAACTCCGTCTCCAAGGGCAACGCGCCGCCACACTTCCG
GAAACGTCGCGTACGGAGGGCGCTGCGATTTTGCGTCACTTCCGCCACCTCTAGCG
>mus_pahari/1-191_Gar-1
(SEQ ID NO: 136)
CCCAAACAACCCCGTCTCCAAGGGCAACGCGTCGCCACACTTCCGGAAACGTCGCGTACGGAGGGCGCTGCGATT
TCGCGTCACTTCCGCCACCTCTAGCG
>oryctolagus_cuniculus/1-191_Gar-1
(SEQ ID NO: 137)
CAACCGTAAACCCCAGCAGAAAGAACAGGCGGAGCCCTAACACCAACCTTCTCCCGGAGACACGCCCCCTGCTGC
ACTTCCGGAATGTTCTGGGGCAAAGGGCGCCGCTATTATACGTCACTTCCGCCGCGGTTCTTTCG
>balaenoptera_musculus/1-191_Gar-1
(SEQ ID NO: 138)
CAGCCGAGCCGCTGGGAGAGGGGCGGTCCCTGACGCCAGCCATCGCCAAGGGCAACGCCGCGGGGCGGCACTTCC
TGCAACGTCACGCTGCCAAGGACGCCGCTATTGTACGTCACTTCCTCCGCTCTCCGTAG
>delphinapterus_leucas/1-191_Gar-1
(SEQ ID NO: 139)
CAAGCCGATCCGCTGGGAGAGGGGCGGTCCCTGACGCCAGCCATCGCCAAGGGCAACGCCGCGGGGAGGCACTTC
CTGCAACGTCACGCTGCAAAGGACGCCGCTATTGTACGTCACTTCCTCCACTTCCCGGAG
>monodon_monoceros/1-191_Gar-1
(SEQ ID NO: 140)
CAAGCCGATCCGCTGGGAGAGGGGCGGTCCCTGACGCCAGCCATCGCCAGGGGCAACGCCGCGGGGCGGCACTTC
CTGCAACGTCACGCTGCAAAGGACGCCGCTATTGTACGTCACTTCCTCCACTTCCCGGAA
>phocoena_sinus/1-191_Gar-1
(SEQ ID NO: 141)
CAAGCCGATCCGCTGGGAGAGGCGCGGTCCCTGACGCCAGCCATCGCCAAGGGCAACGCCGCGGGGCGGCACTTC
CTGCAACGTCACGCTGCAAAGGACGCCGCTATTGTACGTCACTTCCTCCGCTCTCCGTAG
>physeter_catodon/1-191_Gar-1
(SEQ ID NO: 142)
CAAACCGAGCCGCTACTAGAGGGGCGGTCCCTCACGCCAGCCATCGCCAAGGGCAACGCCGCGGGGCGGCACTTC
CTGCAACGTCACGGCGCAAAGGACGCCGCTATTGTACGTCACTTCCTCCGCTCTCCGTAG
>bos_grunniens/1-191_Gar-1
(SEQ ID NO: 143)
CTTGCTGGGCCGCGGGGAGAGGGGCGGACCCTGACGCCAGTCATCGCCAAGGGCAACGCCGCAGAGCGGAACTTC
CTGCAACGTCATGCTTCCAAGGACGCCGATATTGTGTGTCACTTCCTCTGCTCGCCGTAG
>capra_hircus/1-191_Gar-1
(SEQ ID NO: 144)
CTTGCCCGGCCGCGGGGAGAGGGGGGGGCCCTGACGCCAGTTATCTCCAAGGGCAACGCCGCAGAGCGGAACTTC
CTGCAACGTCATGCTTCAAAGGACGCTGATATTGTATGTCACTTCCTCTGCTCGCCGTAG
>ovis_aries/1-191_Gar-1
(SEQ ID NO: 145)
CTTCCCGGGCCGCGGGGAGAGGGGCGGGCCCTGACGCCAGTTATCTCCAAGGGCAACGCCGCAGAGCGGAACTTC
CTGCAACGTCATGCTTCAAAGGACGCTGATATTGTATGTCACTTCCTCTGCTGGCAGTAG
>ovis_aries_rambouillet/1-191_Gar-1
(SEQ ID NO: 146)
CTTGCCGGGCCGCGGGGAGAGGGGGGGGCCCTGACGCCAGTTATCTCCAAGGGCAACGCCGCAGAGCGGAACTTC
CTGCAACGTCATGCTTCAAAGGACGCTGATATTGTATGTCACTTCCTCTGCTGGCAGTAG
>cervus_hanglu_yarkandensis/1-191_Gar-1
(SEQ ID NO: 147)
CTGGCCGGGCGGCGGGCAGAGGGGGGGGCCCTGACGCCAGTCGTCGCCAAGGGCAACGCCGCAGAGCGGAACTTC
CTGCAACGTCATGCTTCAGAGGACGCCGATATTGTATGTCACTTCCTCTGCTCGCCATAG
>catagonus_wagneri/1-191_Gar-1
(SEQ ID NO: 148)
CCCGCCTGGCCACTGGGAGAGGGGCAGTCCCTGACGCCAGTCATCGCCAAAGGGCAACCCCGCGGGGTTCCTGCA
AGCAACGTCATGCCGCAAAGGACGCCGCTATTTTACGTCACTTCCTCTGCTCCCGTTAG
>sus_scrofa/1-191_Gar-1
(SEQ ID NO: 149)
CCCGCCTCGCCACTGGGAGAGGGGCGGTGCCTGATGCCAACCATCGCCAAGGGCAACCTCGCGGGGCAGAAGTTC
CGGCGAGTAACGTCATGCCGCAAAGGACGCCGCTATTTTACGTCACTTCCTCTGCTCCCATTAG
>camelus_dromedarius/1-191_Gar-1
(SEQ ID NO: 150)
CCCGCCGGGCTGCTGGGAGAGAGGCGGTCCCTGACGCCAGCCATCTCCAAGGGCAACCCCGCGGCGGCACTTCCT
GCAGCGCCCTAAGGTAAAAGACGCCGCTATTGTACGTCACTTCCTTTGCTCGCGGTAG
>equus_caballus/1-191_Gar-1
(SEQ ID NO: 151)
AACCCGGGCGCCGGGAGAGGGCGGACCCCTGACGCCGCCGTCACCAGGGCAACCCTGCGGGCACTTCCTGCAACG
TCGCGGCAAAGGACGCCGCTATTACACGTCACTTCCTCTGCTCGTCGGTAG
>canis_lupus_dingo/1191_Garl
(SEQ ID NO: 152)
CCGCCAGGTCCCCGGGAGAGGGGGGCGGAACTCTCACGCCAACCATCTCCCGGGGCAACAGCGCGGCCGCACTTC
CGGGAACTTCTCGACTCAACGGACGCCACTATTATACGTCATTTCCTCCGCTCCTCGTAG
>canis_lupus_familiaris/1-191_Gar-1
(SEQ ID NO: 153)
CCGCCAGGTCCCCGGGAGAGGGGGGCGGAACTCTCACGCCAACCATCTCCCGGGGCAACAGCGCGGCCGCACTTC
CGGCAACTTCTCGAGTCAACGGACGCCACTATTATACGTCATTTCCTCCGCTCCTCGTAG
>rn6_Gar-1
(SEQ ID NO: 154)
AGGCCTGACGATAGAGCCGAAGAACCCAAACCACCCCTGTCTCCAAGGGCAACGCGGCACCACACTTCCGGAAGC
GTCGAGTACGGAAGGCGCTGCTATTTTGCATCATTTCCGCCACCCCTAG
>hetGla2_Gar-1
(SEQ ID NO: 155)
CACGCCCCACTCCGGGAGAGGAGCCGGGTCTCAGACGCCTGCGGTCTCCAGGGGCAACACCGCACAACGCTTCCG
TAAACGTCATGTGCAAGGGACGTCGTTACGTCACTTCAGCGCGCCTTCCTGG
>cavPor3_Gar-1
(SEQ ID NO: 156)
CATGCGGCGTTTCGGAAGAGGAGCCCGCTTCCGGACGCCCGCCGTCTCCAGGGGCAACACTTCCGTGAACGTCAT
GTGTAAGGGACGGGTTACGTCACTTCCTGTGCTCCTTGG
>chiLan1_Gar-1
(SEQ ID NO: 157)
CATGCCCAATTCTGGAAGAGGAATCGCGTCCCTGACGCCTGTTATCTCCAGGGGCAACACTACGGCAATACTTCC
GTAAACGTCATATGTAAGGGACGCTAAACGTCACTTCCTGTACTCCTTGG
>octDeg1_Gar-1
(SEQ ID NO: 158)
CGTGCCTAACTCCGGAATTGGACCCGCGTTCCGGACACCGCTGTTTCCTGGGGCAACACTTCCGTAAACGTCATA
AGCAAGGGACGGCGACGTCACTTCCTGTGTTCCGCGG
>ochPri3_Gar-1
(SEQ ID NO: 159)
AAGGGCGAGCCCCGGGCTGACGGGCGGATCCCCAATGCCCTCCATCTCCCGGAGCAACTCGGCACTTCCGCAAAG
TTCCGCGGCCAAGGACGCCGCTTTTGTGCGTCACTTCCGCCGCTGGACGCGGG
>susScr3_Gar-1
(SEQ ID NO: 160)
CCCGCCTCGCCACTGGGAGAGGGGCGGTGCCTGATGCCAACCATCGCCAAGGGCAACCTCGCGGGGCAGAAGTTC
CGGCGAGTAACGGCATGCCGCAAAGGACGCCGCTATTTTACGTCACTTCCTCTGCTCCCATTAG
>vicPac2_Gar-1
(SEQ ID NO: 161)
CCCGCCGGGCTGCTGGGAGAGAGGCGGTCCCTGACGCCAGCCATCTCCAACGGCAACCCCGCGGCGGTACTTCCT
GCAGCGCCCTAAGGTAAAGGACGCCGCTGTTGTACGTCACTTCCTCTGCTCGCGGTAG
>camFerl_Gar-1
(SEQ ID NO: 162)
CCCGCCGGGCTGCTGGGAGAGAGGCGGTCCCTGACGCCAGCCATCTCCAAGGGCAACCCCGCGGCGGCACTTCCT
GCAGCGCCCTAAGGTAAAGGACGCCGCTATTGTACGTCACTTCCTCTACTCGCGGTAG
>turTru2_Gar-1
(SEQ ID NO: 163)
CAAGCCGATCCGCTGGGAGAGGGGCGGTCCCTGACGCCAGCCATTGCCAAGGGCAACGCCGCGGGGCGGCACTTC
CTGCAACGTCACGCTGCAAAGGACGCCGCTATTGTACGTCACTTCCTCCGCTCGCCGTAG
>orcOrcl_Gar-1
(SEQ ID NO: 164)
CAAGCCGATCCGCTGGGAGAGGGGCGGTCCCTGACGCCAGCCATCGCCAAGGGCAACGCCGCGGGGCGGCACTTC
CTGCAACGTCACGCTGCAAAGGACGCCGCTATTGTACGTCACTTCCTCCGCTCGCCGTAG
>panHodl_Gar-1
(SEQ ID NO: 165)
CTTGCCGGGCCGCGGGGAGAGGGCGGGCCCTGACGCTAGTTATCTCCAAGGGCAACGCCGCAGAGCGGAACTTCC
TGCAACGTCATGCTTCAAAGGACGCTGATATTGTACGTCACTTCCTCTGCTCGCAGTAG
>dasNov3_Gar-1
(SEQ ID NO: 166)
GCCGCCAGGGACTGGGAGGAACAGCCTAATTCCCAACACCTCCCGTTTCCTAGGGCAACAAAGCGGCGTCACTTC
CTGTAACGCCCTGACGCAAAGGACGTTGCCATCCTACGCCACTTCCGCTACTCTCCGGTAG
>jacJacl_Gar-1
(SEQ ID NO: 167)
CAGGGGGGAAGGGAACCCCGGCGCCAGCATCTCCCAGGGCAACGCGGCAAGCACTTCCGGGGGGAGTCTGGAGAC
GGAGACGCCGTTATTTTACGTCACTTCCGCTGTCGCTCT
>eleEdw1_Gar-1
(SEQ ID NO: 168)
TTTAGAAAAAAAATTGGACCACTAACGCCAGGCATCTCCAAGGGCAACAAAGCCGTCCCACTTCCTAACGTCATC
AGGAAAGGCACGCTGTGCTTACGTCATTTCCTTTGCTTGACGGCAG
>tupChil_Gar-1
(SEQ ID NO: 169)
GGGAGGGGCGGCGCCCGGGGCCAGCTGTCTCCCGGGGCAACCTCGCGGGGCGCTTCCGGCGACGCCATGCAGCCA
CGGACGCCGTGACGTCACTTCCGCCACGCAGCGCCGG
>ancestral_sequences4/1-143_Gar-1
(SEQ ID NO: 170)
CCTGCCCCGCCTCTGGGAGAGAAGGCGGATCCCTAACGCCAGCTATCTCCAAGAGCAACATTGCCGCAGCACTTC
CGGGACGTCGTGCTGCAAAGGACGCCGCTGTTATACGTCACTTCCACGGCTCAGCGTTAG
>ancestral_sequences7/1-143_Gar-1
(SEQ ID NO: 171)
CCTACCCCGCCTCTGGAAGAGAAGGCGGATCCCTAACGCCAGCTATCTCCAAGAGCAACATTGCCGCAGCACTTC
CGGGACGTCGTGCTGGGACGCAGCTATTATACGTCACTTCCACGGCGCCGCGTTAG
>ursus_thibetanus_thibetanus/1-191_Gar-1
(SEQ ID NO: 172)
CCGCCAGGTCCCCAGGAGGGGAGGAGGGGGTGTTCACTAACGCCAGCCATCTCCCAGGGCAACACCGCGGCGGCA
CTTCCTGCAACTTCTTGATTGAAAGGACGCCACCATTATACGTCATTTCCTACGGAGGCGTAG
>zalophus_californianus/1-191_Gar-1
(SEQ ID NO: 173)
CCGCCAGGCCTCCGGGAAAGGGGGCGGATCACTAATGCCAGCCATCTCCCAGGGCAACACCGCGGGGGCACTTCC
TGCAACTTCTTGATTCAAAGGACGCCACTATTATACGTCATTTCCTATGGAGGACTAG
>mandrillus_leucophaeus/1-143_Gar-1
(SEQ ID NO: 174)
CCCACCCCGCCTCTGGAAGAGAAGGCGGATCCCTAACGCCAGCTATCTCCAAGAGCAACATTGCCGCCGCACTTC
CGGGACGTCGTGCTGCGGAGGACGCAGCTATTATGCGTCACTTCCACGGCGCGGCGTTAG
>dipodomys_ordii/1-143_Gar-1
(SEQ ID NO: 175)
CCCGCTCCGCCTCCGGCAACAGCCATCTCCACCGGCGCCAACGCCGCGGCACTTCCGGGACGCCTCGGCGCGAAG
GACGCGGACCTTTGACGTCACTTCCGCCGCCCTCAGGAG
>chinchilla_lanigera/1-143_Gar-1
(SEQ ID NO: 176)
CATGCCCAATTCTTGGAAGAGGAATCGCGTCCCTGACGCCTGTTATCTCCAGGGGCAACACTACGGCAATACTTC
CGAAACGTCATATGTAAGGGACGCTAAACGTCACTTCCACTCCTTGGCG
>octodon_degus/1-143_Gar-1
(SEQ ID NO: 177)
CGTGCCTAACTCCGGGAATTGGACCCGCGTTCCGGACACCGCTGTTTCCTGGGGCAACACTTCCGTAAACGTCAT
AAGCAAGGGACGGCGACGTCACTTCCTGTGTTCCGCGGCG
>fukomys_damarensis/1-143_Gar-1
(SEQ ID NO: 178)
NNNNNNNNNNNCCCGGGAGAGGAGCCGGGTCCCAGACCTCTGCGGTCTCCAGGGGCAACGCCACGCAACACTTCC
GAAACGTCATGTGCGAGGGACGCTGTGCTCACTTCCGGTGGGCCACTG
>heterocephalus_glaber_female/1-143_Gar-1
(SEQ ID NO: 179)
CACGCCCCACTCCAGGGAGAGGAGCCGGGTCTCAGACGCCTGCGGTCTCCAGGGGCAACACCGCACAACGCTTCC
GAAACGTCATGTGCAAGGGACGTCGTTACGTCACTTCCGCGCCTTCCTG
>ictidomys_tridecemlineatus/1143_Garl
(SEQ ID NO: 180)
CACGCCCGACTTCTGGGAGAGGAGGCGGGTCCCTAACTCCGCTATCTCCTAGGGCAACTCGACGGCAATACTTCC
GGAACGTCCTGACGTAATGGATGCCGTTTCGCTTTACTTCCGCTTTCTCTTGCTAA
>spermophilus_dauricus/1-143_Gar-1
(SEQ ID NO: 181)
GCCCGACTTCTGGGAGAGGAGGCGGGTCCCTAACTCCGCTATCTCCTAGGGCAACACGTCGGCAATACTTCCGGA
ACGTCCTGACGTAATGGATGCCGTTTCGCTTTACTTCCGCTTTCTCTGGCTAA
>urocitellus_parryii/1-143_Gar-1
(SEQ ID NO: 182)
GCCCGACTTCTGGGAGAGGAGGCGGGTCGCTAACTCCGCTATCTCCTAGGGCAACACGACGGCAATACTTCCGGA
ACGTCCTGACGTAATGGACGCCGTTTCGCTTTACTTCCGCTTTCTCTTGCTAA
>jaculus_jaculus/1-143_Gar-1
(SEQ ID NO: 183)
NNNNNNNNNNCCCAGCGGGGGAAGGGAACCCCGGCGCCAGCATCTCCCAGGGCAACGCGGCAAGCACTTCCGGGG
GGAGTCTGGAGAAGACGCCGTTATTTTACGTCACTTCCGCTGTCGCTCTAG
>myotis_lucifugus/1-143_Gar-1
(SEQ ID NO: 184)
GAGAGAGCCGGTCTCCACCTCCGGGGATATCCCGGGGCAAAGCCGCGGTGACACTTCCGGAACGTCAGGATGCCA
CGGACGCGGCTGTTTTACGCCACTTCCTTGGCTTGTCGGAAG
>pteropus_vampyrus/1-143_Gar-1
(SEQ ID NO: 185)
GGAGAAGGGTGGGGCCTCACCCCAGACGTTTCCTAGGGCAACACCACGGCGGCACTTCCGGAACGTTGAGATGCA
ACGGACGCCGCTATTATACGTCACTTCCTCGGCTCGTCGATAG
>choloepus_hoffmanni/1-143_Gar-1
(SEQ ID NO: 186)
ACCGCTCGGGGCCTAAGAAAGATTCTTAACGCCAGTCACCTCCAAGAGAAACAGAGCAGTTGCTCTTCCTGAACG
CCACGACGCAAAGGGCGTTGCCATTGTACGTCACTTCCTCAACTCTCTGGCAG
>dasypus_novemcinctus/1-143_Gar-1
(SEQ ID NO: 187)
GCCGCCAGGGAGCTGGGAGGAAAGCCTAATTCCCAACACCTCCCGTTTCCTAGGGCAACAAAGCGGCGTCACTTC
CTGAACGCCCTGACGCAAAGGACGTTGCCATCCTACGCCACTTCCGCTACTCTCCGGTAG
>procavia_capensis/1-143_Gar-1
(SEQ ID NO: 188)
TTCTCCAGGCTCCTGGATGAAGGGGCGGATCCTTAACGCCAACCATCTCCAACGGCAACAACGCAGGGGCACTTC
CTTTACGACAGGACGCAACGGAAGCTCTTGGCGTACGTCACTTCTGCTTGTCAG
>equCab2_Gar-1
(SEQ ID NO: 189)
CCCGGGCGCCGGAGAGGGCGGGACCCCTGACGCCGCCGTCACCAAGGGCAACCCTGCGGGCACTTCCTGCAAACG
TCGCGCCAAAGGACGCCGCTATTACACGTCACTTCCTCTGCTCGTCGGTAG
>cerSiml_Gar-1
(SEQ ID NO: 190)
CCCCCGGGCCGCCGGGAGGGGGTAGACCCCCGACGCCGGCCGTCACCAGGGCAACAGCGCGCGGCACTTCCTGCA
ACGCCGCGAGGCAGAGGACGCCGCCATTATACGTCACTTCCTCTGTTCGTCGGGAG
>felCat8_Gar-1
(SEQ ID NO: 191)
CCGCCGGACCCCCGGGAGAGGGAGCGGATCACCAACGCCAACCGTCTCCCAGGGCAACACCGAGGCGGCACTTCC
GGCAAGGTCTGGATTCAAAGGACGCCACCATTATACGTCATTTCCTCTGCTCCTCAGTAG
>mus_Furl_Gar-1
(SEQ ID NO: 192)
CCCGCAGGCTCCCGGGAGAGGGGGCGGATCACTAACGCCAGCCATCTCCCAGGGCAACAGCCTGATGGCACTTCC
TGCAGCTTCTTTGCAGTCAAAGGACGCCACTATTAAACGTCACTTCCTACGTAGGTGAAG
>ailMell_Gar-1
(SEQ ID NO: 193)
CCGCCAGGTCCCCAGGAGGGGAGGAGGGGGAGTTCACTAACGCCAGCCATCTCCCAGGGCAACACTGCGGCGGCA
CTTCCTGCAACTTCTTGATTGAAAGGACGCCACCATTATACGTCATTTCCTACGGAGGCGTAG
>odoRosDivl_Gar-1
(SEQ ID NO: 194)
CCGCCAGGCTTCCGGGAAAGGGGGCGGATCACTAACGCCAGCCATCTCCCAGGGCAACACCGCGGGGGCACTTCC
GGCAACTTCTTGATTCAAAGGACGCCACTATTATACGTCATTTCCTATGGAGGACTAG
>lepWed1_Gar-1
(SEQ ID NO: 195)
CCGCCAGGCCTCCGGGAAAGGGGGCGGATCACTAACGCCAGCCATCTCCCAGGGCAACACCGCGGCGGCACTTCC
TGCAACTTCTTAGATTCAAAGGACGCCACTATTATACGTCATTTCCTACGGAGGACTAG
>pteAlel_Gar-1
(SEQ ID NO: 196)
CCTGCAGGGCTGCTAGGAGAAGGGCGGGGCCTCACCCCAGACGTTTCCTAGGGCAACACCACGGCGGCACTTCCG
GCAACGTTGAGATGCAACGGACGCCGCTATTATACGTCACTTCCTCGGCTCGTCGATAG
>pteVaml_Gar-1
(SEQ ID NO: 197)
CCTGAAGGTCTGCTAGGAGAAGGGTGGGGCCTCACCCCAGACGTTTCCTAGGGCAACACCACGGCGGCACTTCCG
GCAACGTTGAGATGCAACGGACGCCGCTATTATACGTCACTTCCTCGGCTCGTCGATAG
>eptFus1_Gar-1
(SEQ ID NO: 198)
CCCACGAGCGGCTGGAAGAGGGCCGGTCTCCACCTCCTCCCTCCCGGGACATCCCGGGGCAACACCGCGGTGACA
CTTCCTGGAACGTCAGGATGCCACGGACGCGACTATTTGACGCCACTTCCTTGGCTTGTCGGAAG
>myoLuc2_Gar-1
(SEQ ID NO: 199)
CCGACCGGCGGCCAGGAGAGAGCCGGTCTCCACCTCCGGGGATATCCCGGGGCAAAGCCGCGGTGACACTTCCTG
GAACGTCAGGATGCCACGGACGCGGCTGTTTTACGCCACTTCCTTGGCTTGTCGGAAG
>loxAfr3_Gar-1
(SEQ ID NO: 200)
CCCTCCTGGCTCCCGGGAGAGGTGGCAGAGCCCTAACGCCATCCATCTCCAAGGGCAACAGCGCAGCGGCACTTC
CTTTAACGTCATGATGCAAAGGACGCTACCTACGTCACTTCCTCTGCCCGTCGTCAG
>triMan1_Gar-1
(SEQ ID NO: 201)
TCCTCCTGGCTCCTAGAAGAGGGGGCGGATCCCTAACGCCAGCCATCTCCAAGGGCAACAACGCGCCGGCACTTC
CTGTAATGATGCAAAGGACGCTGCTGCCGTACGTCACTTCCTTGACTCGTCGGTAG
>chrAsil_Gar-1
(SEQ ID NO: 202)
ACCTCCGGGCCTCTGGGAGAGGGGAGGATTCCTAACGCAGGTCGTTTCCAAGGGTAACAACGCAGCGGCACTTCC
TTCAACGTGTGGACGCAACGGACGCTGCACGTCACTTCCGCTGCCTGTCCGTTG
>oryAfel_Gar-1
(SEQ ID NO: 203)
TCCTTCAGGCTGTTGGGCGTGGGGGCGGATCCCTAACGCCAGCCATCTCCAAGGGTAACAACGTGTGGGCACTTC
CACACGTCATGATGCAAAGGCCATTACTATTGTACGTCACTTCCTCTGCTTGTCGGTAA
>mouse_7sk-1
(SEQ ID NO: 204)
GAGAGTAAGCAGGCTCTTGGTAGGTATATAAGGCCATAGAATTTTGTAACTTTACACATGTGGTGACCTTATGTA
GCCGACTGTACTTGATATTATAACAAATCCTGAATCCGTTTTAGGGTTAAATAATCCTTTTTATACTCGCTTCGT
TCTAAGTTTAAATTAAAATACTTAAATTTAGGATGTTTTTACTGTTAACCAAAATGCTTTGGGGCTATGCAAAAT
ACAACAGTTTGGATTGGTTAAACCTTCCGAAGCCCCGCCCCCGACGGCCATGTCT
>CD2AP_Bidirectional_Promoter
(SEQ ID NO: 205)
AGCGAGCCCAAGCTCCTCTGCACCGCTTCCTCATCCGCTCGCTGCACCTGGACGCGGTCGGCGCGCGACCCCCGG
CCGTGACGTCACCGCACCTGGCAGCAGCCGTGGGGACCGGGAGAGAGCCCGAACGCGACGGGGGGGGGTGGGGCG
GGGAGAACGAGGGCGTTCTCGCGAGATTTGCCTCCTCCCGGTCCCAGCTCCCCGCACCTTCTCGGCCTCTGTCTG
GGTCCCCACCTTAGTCTACGGTGTCGCCTTTTCTAACTGCGAGTGCTAAGGAAGAGGCGAGGGGGGGGCTCCGAG
GCTAGGCGGGCGCTCGGGGTTGGAGCCGAGGGTCTGGGCAAACCGGTGGGTCCCTCCCCACTGCGGGAGCGGCCA
GGGTGGGAAAACCGCGGTCGGGCGGGGGGGGTAGGGCCCTCCCGCCGCCGTGGCTCCTGGGGAGGCCAGGGGTGA
GGAGCTGTCGCCGCCTTTGCCTCTGCCTCGAGGGCCGCGCTGAAGAGACTGGTAGGAGAGCGCCGCGGGCGGATG
GAGGCGACTCTTCGCCCCGCCTGAGCTCAGGAGGGGCTAGCGCGGAGCGCGGGTCCCGCCTCCAGCCGCGGGAGC
GGCCGCGCGAGCCACCACTGGAGGAGGAGGAGGAGGAGCGGACGTCGGCTTCTCCCCGCGGGAGCCCCCAGC
>DCTN6_Bidirectional_Promoter
(SEQ ID NO: 206)
ACGCGACGCAAACAAGAGTCGCAAGCTTCCGGGTCCCCGCCCCACCCCGGCTCCGCCCCTCCCCCAACCCTGCCA
GGCTCTCCAATCGCATGTGGAATTATCGCTCTACCCAGGCGGTGGTGTCGATCTACGTTCCAATTGGGGCCGTAC
C
>EMBP1_Bidirectional_Promoter
(SEQ ID NO: 207)
AAAACCTTACACCTGCGCAAAAATAAGCCTCCCTCATAAGAAAGCCCAAAGATGTCCGGGGTCGGGGAGGAGGAA
AGTGTCTCTCATCTGTCCCATCAACGAAAATTAGTGAAATCTGCCTCAGATGAAGTGCAAAGGCCAGTCTGCAGG
GATAGTTTCAACCTCTCCCCACGCGATGGGCTACACATCACCTGCCCAAGCTCTCTCCCGACCTGCTAGAGCCTA
GAGGGCGGAGGCCGGAGAGGCTGCAGCCGGGAGTAGCACCGCACATCCGGGAACGCC
>EP400NL_Bidirectional_Promoter
(SEQ ID NO: 208)
ACCCGTCTACAGTGGACACGACGAAACCAGGGACATGTCCCACCATTTCAGTGGTCACAGGCAAGAGTCTTGTGG
ATCTTCGGATCCCACGTAACATCTCATCTCCCTAGGCACCCCGACTCCCCTGCCCAATTTAAAACAGACCTCAGC
CTGCCCCATCCCGGCTGCTTTGCCTGGTGCTCTTCTAACTGCATGTTTATCTATCCTCCCCGCCTAGACTGTAGG
GCCCGCGAGGGGAGCCGCTAGCTGTGCTTGTCAGTGTGACCAGCGCTCAGCAGGTGTCCGGCGGGAGGGCGGGCA
AATACAACTCAGTGCCCACGTGCGAATGAATGAACAAACTAGTTCCGGGCGGAGCCAGAGGCGCGCGCCGGCGCG
GACCGAGGCCCGGCCCTATCCGCCCCGCCCCCTCCGCCCCGCCCCCTCCGCCACGTCCCTCCGGGTCCGCTGGGC
GCTGATTGGTCCGAGCCTCGCCTGCGCAGTGCCGGGCCGGCTCCCGCGCTTGC
>FCHO21_Bidirectional_Promoter
(SEQ ID NO: 209)
CCGACTCCACTGCCGCTGGCTGGCCCTTCTCTTCCCTCTGTCCCTGGGCCAGTGCCCGTCGCACCACAAACAGTG
CGAGCAGTCTCCCCGGTGACTCCTCAAGGACCCAGTTCTCCACCATTCCTAAGAGAACACTCAACCCAGCCGCGC
CCGGGATGCAGAGAGATCTACCAACACCCGAGAATGGGGACAGGGCGCATGCGCACACCGTGGCCGTGGCGTCTA
AGTGCTCGCCCAGCTGCGGCAGCCGCTAGGTGGCGCATGCGCCCTGGAAGGTGCGGGCCGGTCTCTGGGAAGAAG
GCGGCGGCGGCGAAAGGCGGGGGTGCTGTGGGGGCCGGGCCGTGTTT
>FCHO22_Bidirectional_Promoter
(SEQ ID NO: 210)
CCGACTCCACTGCCGCTGGCTGGCCCTTCTCTTCCCTCTGTCCCTGGGCCAGTGCCCGTCGCACCACAAACAGTG
CGAGCAGTCTCCCCGGTGACTCCTCAAGGACCCAGTTCTCCACCATTCCTAAGAGAACACTCAACCCAGCCGCGC
CCGGGATGCAGAGAGATCTACCAACACCCGAGAATGGGGACAGGGCGCATGCGCACACCGTGGCCGTGGCGTCTA
AGTGCTCGCCCAGCTGCGGCAGCCGCTAGGTGGCGCATGCGCCCTGGAAGGTGCGGGCCGGTCTCTGGGAAGAAG
GCGGCGGCGGCGAAAGGCGGGGGTGCTGTGGGGGCCGGGCCGTGTTTACACAGCGGCGGGCGGGCGCGGACGCGG
AACCCGGCGCGGCGGCGGCACG
>KMT5C1_Bidirectional_Promoter
(SEQ ID NO: 211)
CGCGGGGGGGGAGGGGAGAGGGATGGCGGTGCGCGCGCATTCACCGCCTCCCTCCCGCCGGGTCTGGCTTTCTCC
CTCCTGTGGCCGAAGCTTTCCTCGGAGAAATAGAAGAGGGAGGCCGCGACTCTATGGTGATGGACGGAGGCCTTA
CCCAATGGAAAGAGGAGCTGTCCCAAGGCCAGGCAATCATATACGACTACTGGAGCTGGCAGAGCCCGCCCTCTT
TCCACTTGGACCTGAATAACCCGACCCAAACCGAGTTTCGCCCGGAGAGACTGCGCTTTCGGCCAATGAGTGCGT
CGATTTCGAGCCCCAGTGTGAGCGAAGGCGGGACAAGTCTCCATGGCAGCGACTAAAGGACAGCGATGTGAACCA
CTGACAACAGTTCGCGGCGTTTGACGGCGGCGGGGGCGTGGCGGGGTTTTATCTGTGTATTGACGAGAGCCGGGC
GCGGAGGGAAAGAGTGGGGCTTGGCCAATGGGAGCGCCGTGAGCTTCGTAGCAACGGAGGAGTGGCGGTGGCTGT
GGCCAATAGAAAGCCTCAGTGGCCTTGGCGGGGCTGGCCCGGAG
>KMT5C2_Bidirectional_Promoter
(SEQ ID NO: 212)
CGCGGGGGGGGAGGGGAGAGGGATGGCGGTGCGCGCGCATTCACCGCCTCCCTCCCGCCGGGTCTGGCTTTCTCC
CTCCTGTGGCCGAAGCTTTCCTCGGAGAAATAGAAGAGGGAGGCCGCGACTCTATGGTGATGGACGGAGGCCTTA
CCCAATGGAAAGAGGAGCTGTCCCAAGGCCAGGCAATCATATACGACTACTGGAGCTGGCAGAGCCCGCCCTCTT
TCCACTTGGACCTGAATAACCCGACCCAAACCGAGTTTCGCCCGGAGAGACTGCGCTTTCGGCCAATGAGTGCGT
CGATTTCGAGCCCCAGTGTGAGCGAAGGCGGGACAAGTCTCCATGGCAGCGACTAAAGGACAGCGATGTGAACCA
CTGACAACAGTTCGCGGCGTTTGACGGCGGCGGGGGCGTGGCGGGGTTTTATCTGTGTATTGACGAGAGCCGGGC
GCGGAGGGAAAGAGTGGGGCTTGGCCAATGGGAGCGCCGTGAGCTTCGTAGCAACGGAGGAGTGGCGGTGGCTGT
GGCCAATAGAAAGCCTCAGTGGCCTTGGCGGGGCTGGCCCGGAGAGCAGATGGGAGGTGCGGCGACAGTGTTTGA
CGAGAGCCGAAGGAGGCTGTGGGAGGTGTTGGCGGCGGCGGCGCGGGCGCCTGAGGAGGAGGAGGAGAAGCGGGT
GAGGGGCGGCGCGGGGCCCGATCTCTGAGCCCCTTCACGGCCCCAGCCCCGCGCCGCCTTGGCTCCCCAGTCGCC
CCCTGCCCCGACTGCCCCCCACCCCGCCCGGCCCCTCCTCGTGTCCAGGCGCCCAC
>LZTR11_Bidirectional_Promoter
(SEQ ID NO: 213)
TGAAGGAGCTGAGGCCCTGCTAAGTAGGAATGAGAATCCAGAGGCTCCTCGCCGGGCTGCCTCTCAGTCAGTAAG
AAAGCCAAGGGGAGAGGGGAGTTGCTGGGGGTCAGGGCTGAGGGCGCTAGCAGGAAAGGGAGCGTTGAGCCGCCT
GCAGAGGCCGCTGCGAGCCCGGAACCCTCCATGGGGGATCCCGGCAGCGGCAGACGATCCAGGCCGGAGCCACGC
GCAGACCCAGGGCATGCCGGGAACTGCGAGCCGGCCGCGGGTCTTCGGGCTGCGTGGGCCTGGGAGGCGCCGGGA
AGAGCAGTCGCGACGGGGCTAGGGACGACACACTGCATTCACTGGAAGGGACAACGCAGCGCCAGTACATAGCCT
GAAACGCTCCCCAGAAGGTCCCACGCTCGCCGCGCGGTCGACAACCGCATCCTGCGCTCGCCCGCGGTGTCTCGG
CAAGCGGTAGGCTTGTCGGGAAGAGCTGGAGGGCGCAAGTGCGGCGCTGGCCGGACGTGCCGC
>LZTR12_Bidirectional_Promoter
(SEQ ID NO: 214)
TGAAGGAGCTGAGGCCCTGCTAAGTAGGAATGAGAATCCAGAGGCTCCTCGCCGGGCTGCCTCTCAGTCAGTAAG
AAAGCCAAGGGGAGAGGGGAGTTGCTGGGGGTCAGGGCTGAGGGCGCTAGCAGGAAAGGGAGCGTTGAGCCGCCT
GCAGAGGCCGCTGCGAGCCCGGAACCCTCCATGGGGGATCCCGGCAGCGGCAGACGATCCAGGCCGGAGCCACGC
GCAGACCCAGGGCATGCCGGGAACTGCGAGCCGGCCGCGGGTCTTCGGGCTGCGTGGGCCTGGGAGGCGCCGGGA
AGAGCAGTCGCGACGGGGCTAGGGACGACACACTGCATTCACTGGAAGGGACAACGCAGCGCCAGTACATAGCCT
GAAACGCTCCCCAGAAGGTCCCACGCTCGCCGCGCGGTCGACAACCGCATCCTGCGCTCGCCCGCGGTGTCTCGG
CAAGCGGTAGGCTTGTCGGGAAGAGCTGGAGGGCGCAAGTGCGGCGCTGGCCGGACGTGCCGCACCGTCAGCGCA
GGGCTCGCCGGGAAATGTGGTTTCTCCAGCCGGCCCGGGGCGGTGGCCGCAAGTTGGGCTTACAGCGCGGCCGAT
CCGGCGTGGACCCGGG
>PATJ1_Bidirectional_Promoter
(SEQ ID NO: 215)
GAGTCGGGGCGAGGGGAGGGCCTGCCAGGTGAGGCGCGGTC
>PATJ2_Bidirectional_Promoter
(SEQ ID NO: 216)
GAGTCGGGGCGAGGGGAGGGCCTGCCAGGTGAGGCGCGGTCACCCTGGGCCTCTCACTTCCGCCCAGGTGAGGCA
GGGCCGACACCGAGCCCGCCCGACCCGGGCTCCCACCTGCTCCTCCAGCGCACCAG
>PCNX11_Bidirectional_Promoter
(SEQ ID NO: 217)
TTCACAAATATCATAAATGACAGGCAGGACGCTTTTCTGGAGTCAAGATCTGTTAGTTTCGGAGTCAGAAAGACC
CCGTTTAGAGACTCGTAGGCGAACTTGCCAGGGGGCCTACCAGGGGCAGAATGGGGTCCTCCGGACCAGCCAGCC
GCGTCTCAGCCACCTCCGCAGCCCCCGGGGCCCTGAACCCCGGCCGCGTTGACGCGCGCTTCTCCCGGACGTCGG
CAGGAGGCGCCCGCGGCGGACCAGGCGCGGCGCGCACCGTAGCCGGCCCAGGGGGGGGAGGGAGCGGA
>PCNX12_Bidirectional_Promoter
(SEQ ID NO: 218)
TTCACAAATATCATAAATGACAGGCAGGACGCTTTTCTGGAGTCAAGATCTGTTAGTTTCGGAGTCAGAAAGACC
CCGTTTAGAGACTCGTAGGCGAACTTGCCAGGGGGCCTACCAGGGGCAGAATGGGGTCCTCCGGACCAGCCAGCC
GCGTCTCAGCCACCTCCGCAGCCCCCGGGGCCCTGAACCCCGGCCGCGTTGACGCGCGCTTCTCCCGGACGTCGG
CAGGAGGCGCCCGCGGCGGACCAGGCGCGGCGCGCACCGTAGCCGGCCCAGGGGGGGGAGGGAGCGGAGAGGAGG
AGCTGGAGGGGGCGCGGCTTCCTCTCGGTCG
>PCNX13_Bidirectional_Promoter
(SEQ ID NO: 219)
TTCACAAATATCATAAATGACAGGCAGGACGCTTTTCTGGAGTCAAGATCTGTTAGTTTCGGAGTCAGAAAGACC
CCGTTTAGAGACTCGTAGGCGAACTTGCCAGGGGGCCTACCAGGGGCAGAATGGGGTCCTCCGGACCAGCCAGCC
GCGTCTCAGCCACCTCCGCAGCCCCCGGGGCCCTGAACCCCGGCCGCGTTGACGCGCGCTTCTCCCGGACGTCGG
CAGGAGGCGCCCGCGGCGGACCAGGCGCGGCGCGCACCGTAGCCGGCCCAGGGGGGGGAGGGAGCGGAGAGGAGG
AGCTGGAGGGGGCGCGGCTTCCTCTCGGTCGCTCCCTGGCGCCGGGCCTCTTTCTCTGCCTGGCCCAGGGCTGGC
GGCCGGCGGGGGTCGCGGCGGCGGCAGTGGGGGCGCTGGCGGGCCGCGGGTGGCGGGGGCCGGGCCGCGGCTCCG
GGTGTTAGGAGACAAGATGGCGGCGGCTCTCAGAAGGCCGGTCTCCTCCTCTCCGCCGTCCTCCGCCCCGCCGCT
CGCCGCCTCCTCCTCTCGGGTCTCCTCCTCCTCGTTTGCTGCCTCCTCCTCCTCCTGCAGCAGCACCAGCGACCG
CCGAAGCGCCGGCTCGCTCACCCGGAGCTCCGGAGGTGGATAGACGGGGCAGCTGCAGGCTCCGGCGACCGAGGC
CGAGCTGGGGCCGGGGGGGGACGGCGGCGGCGGCGGCGGCGACGGCGGCGGCGCCGGGTGGGG
>PTGERN_Bidirectional_Promoter
(SEQ ID NO: 220)
AATTTTTGGCATAGGCCAAGCGGCTGGTTGGTGGGGTGTTTAGCTCAGGACGAGAGGCCGAACGAGCGGGGAGTT
GGCTGAGGATAGACTAGACACGCGTGGGTGACTCCAGCGTGATGGAACGCGGGGTGTCCCGGGATAGGGCTAAAG
CGATGGGATTTCCAGACGAGTCTTTCCCAGGCCAACTTTTAAAGGTCGGAGGAAAGTTTCTCGTGGGGTGGGGGC
CCAGAGGGGATGGCAGGGTGGGCTCCGACGCCTCCTCGCCTTTAAGCGGGTGGCCCCGGCTCTTCCTCCGTTACC
TGGAGCGGGGGGGGCTTGGGAAAGTTTGTGTTTGTTGCTGGCAAAGCGCCGGATGGGAGGCGCGGGCGGGCGCT
GCGGTTCTTCCCTTCT
>RMRP_Bidirectional_Promoter
(SEQ ID NO: 221)
ACGTCCTCAGCTTCACAGAGTAGTATTTTATAGCCCTAAAGAAATTGTGTTTTATGATTAGGGTGAGAAAGTTGG
TGGCGTGAGATTAAAAAAACCGTTTTCGGGCATAACTTTCTAAGACTATAGGCTTTCAGAGGCATTGTGGCTAGC
AGAATAGCTAATAGACACGAAATGAACAAATACAGGAAAGCTAGAATGACACTATCTTATGCAAATATGGTCTGG
CCCCGCCCTACGGGGAGTGGGCGTGGCCTCCCCGGAGCCGGCCGGCCTGCTCGCGTGCGCGTGCGCGTTGGGGCG
GCCGGCCAATGCCGGACCGCTTCGGCACCGCCCGCCCGATCCCTCCACCCGTGGGCCGGCA
>RNF1871_Bidirectional_Promoter
(SEQ ID NO: 222)
CCAGGACCTTGCAGGTGGAGAGCATAGTTGCCAAAATCAAGGCGGAGGAGCGCACCGCCGCTAGGATCCAGGCGG
AGAAGCCCACCGCGGCCAGGACCTAAGGATGCAGTACACTGCTGCCAGGATCTTGTCTGTGGAGCGCAGCGCGGC
CAGGACCTCCGGCTGCAGCACACCGCTGCCAGGATCTTATCGGCAGAGCGCTCCGCGGTCCGGACCCCGCCCCGT
GCGCGTCCCCGACCCCGCCCC
>RNF1872_Bidirectional_Promoter
(SEQ ID NO: 223)
CCAGGACCTTGCAGGTGGAGAGCATAGTTGCCAAAATCAAGGCGGAGGAGCGCACCGCCGCTAGGATCCAGGCGG
AGAAGCCCACCGCGGCCAGGACCTAAGGATGCAGTACACTGCTGCCAGGATCTTGTCTGTGGAGCGCAGCGCGGC
CAGGACCTCCGGCTGCAGCACACCGCTGCCAGGATCTTATCGGCAGAGCGCTCCGCGGTCCGGACCCCGCCCCGT
GCGCGTCCCCGACCCCGCCCCGTGCGCGTCCCCGGCGTTGGCGTCTTCGTCCTGTTGCTGGTCTCCGTCCGGTCG
CCGGCCGTCTAGGTCTCCGGCCCTCCCCAGCCGCTCCTGCGCCCTTGCCGGCCCCGCCGCCCGCAGC
>SAMD4B1_Bidirectional_Promoter
(SEQ ID NO: 224)
CGCCCACTGAGGACAGCCTTGGGTGAGGCGGGCCACCCAAGGGGGGGGGAAGAGGAGGCCTGGAACGCCTGAATC
AGGAACTGTGACTTCGCTCGGGGCAGCTGGGGTGGACGCGCGCGAGCCTGCCCCCTGCGGGCCTGGAGGCCCAAC
CTCAGACTCCGCCGGGCCCGTTGCCCTGGGCAACGCCCCGCGCGCCCCGCCCCTTCCCCGCCCCCCAGCCCCAAA
CCCCAGGCCTGGCCGACTGCCCGTCACCCCCACGTCCGACCAATCCCGCCGAGGAGGGGGCGGGCCTCTTGGGCC
CCGTTCCACCACCGTCGCTCCCCCCTCGCCGCGACCCCGCCTTACTCGGCTCACACCTCCCGCCCTTCGGGCTGC
CCTCGCCGCCCGTTGGCTGGCGCGCCGTTCGTCACCCGGGCGTGAGCTAATGCCGGCGCGCGGCGGCCCCCGTCG
GGGCGGGGCCAGGGGCGGTGACGCACGGCGCGGTGACGCAGCGCGACGGCGGCGGCGGCGGC
>SAMD4B2_Bidirectional_Promoter
(SEQ ID NO: 225)
CGCCCACTGAGGACAGCCTTGGGTGAGGCGGGCCACCCAAGGGGGGGGAAGAGGAGGCCTGGAACGCCTGAATC
AGGAACTGTGACTTCGCTCGGGGCAGCTGGGGTGGACGCGCGCGAGCCTGCCCCCTGCGGGCCTGGAGGCCCAAC
CTCAGACTCCGCCGGGCCCGTTGCCCTGGGCAACGCCCCGCGCGCCCCGCCCCTTCCCCGCCCCCCAGCCCCAAA
CCCCAGGCCTGGCCGACTGCCCGTCACCCCCACGTCCGACCAATCCCGCCGAGGAGGGGGCGGGCCTCTTGGGCC
CCGTTCCACCACCGTCGCTCCCCCCTCGCCGCGACCCCGCCTTACTCGGCTCACACCTCCCGCCCTTCGGGCTGC
CCTCGCCGCCCGTTGGCTGGCGCGCCGTTCGTCACCCGGGCGTGAGCTAATGCCGGCGCGCGGCGGCCCCCGTCG
GGGCGGGGCCAGGGGCGGTGACGCACGGCGCGGTGACGCAGCGCGACGGCGGCGGCGGCGGCGGCGGCGGTGGTC
GGTGCGGGAGGAGGGAGGGGAGCTTGCGGGCCCGAGA
>SAMD4B3_Bidirectional_Promoter
(SEQ ID NO: 226)
CGCCCACTGAGGACAGCCTTGGGTGAGGCGGGCCACCCAAGGGGGGGGGAAGAGGAGGCCTGGAACGCCTGAATC
AGGAACTGTGACTTCGCTCGGGGCAGCTGGGGTGGACGCGCGCGAGCCTGCCCCCTGCGGGCCTGGAGGCCCAAC
CTCAGACTCCGCCGGGCCCGTTGCCCTGGGCAACGCCCCGCGCGCCCCGCCCCTTCCCCGCCCCCCAGCCCCAAA
CCCCAGGCCTGGCCGACTGCCCGTCACCCCCACGTCCGACCAATCCCGCCGAGGAGGGGGCGGGCCTCTTGGGCC
CCGTTCCACCACCGTCGCTCCCCCCTCGCCGCGACCCCGCCTTACTCGGCTCACACCTCCCGCCCTTCGGGCTGC
CCTCGCCGCCCGTTGGCTGGCGCGCCGTTCGTCACCCGGGCGTGAGCTAATGCCGGCGCGCGGCGGCCCCCGTCG
GGGCGGGGCCAGGGGCGGTGACGCACGGCGCGGTGACGCAGCGCGACGGCGGCGGCGGCGGCGGCGGCGGTGGTC
GGTGCGGGAGGAGGGAGGGGAGCTTGCGGGCCCGAGAGGGGGCGACGGCGGCGGCGGTGGCCTGAGGAGGCCCGA
GCGGCGGCGGTGGCGGCGAAGGCCGAGGCG
>SETDIA1_Bidirectional_Promoter
(SEQ ID NO: 227)
CGGAGGCGCCCCCTAGTCCCAGGCTCTGCACGCCCTGGCCCCGCCCCTTGACTCGGCCCCGCCCACAGCGGAATC
CGCAGATTCGCCAGGTCGG
>SETD1A2_Bidirectional_Promoter
(SEQ ID NO: 228)
CGGAGGCGCCCCCTAGTCCCAGGCTCTGCACGCCCTGGCCCCGCCCCTTGACTCGGCCCCGCCCACAGCGGAATC
CGCAGATTCGCCAGGTCGGATCCTCAGAATTCCTCGGGTCCCTCGATACTCGGCTGAAAATTCTCATCGGACTCT
GAGAGGAGCGCTGGGCTGGAGGCATTTTCCCCAGGGACAGAAGCGGGCTATTCTCTCACTTGGGCCAGTAAGAAA
AATCCAAAAAAAGTTGTCGACTCTGCCAGCAGGGATTGGCTAACGGGCCGTTATTTTCTTGACTCCACCAAGGCG
GATGAAGGGGAGGCTACGGCTGAGGCCGGGAACAGTGGCGAATCTGCAGCCTCTCAGAATTTGGCAGTGCAAGGA
AGGGACGGGGAAGAGAAGCAAAGCGGCGCGCATCCTGTCCAGCGATTCGCCCCGCCCGCCCGGTGAATCTGCGTC
TGCAGAACGCGCCACTGAAGGTTCCCCAGCGCTGGCTGGCCTCCTCCCCTCCGCCCCGCCCCTTTTCCTCAGGGA
CTAGTCGCAGCTTTCGTCGCCGCCGATTCGTCAAGGTCCCGGGCCGCAGCATCTAGATCGTCGTGGCGAAGCCGA
CTCTCCGGGGGATGCGGCCAATCTCCAAGCTCCCTGGGCCGCAACTTCCGAGCCTCCCAGGGCGCCGGCCGAGGC
GAAGCCGCTACCCTCGGCCCCGTGGGTCCCCCGGCAGCGCCTGTGGCGAAA
>SNORD651_Bidirectional_Promoter
(SEQ ID NO: 229)
GATATCTTTTTTTTTTGAAGCGAGTTTTAACAAGATCAGCTGTTTATTCATTCCACTATGGGGTTGAAGGGATCA
TTGGCCAGCTCAAGGCTTACCTTCTCTTGGGCTGAGATGCTGCTGCCAGCTCTAAAACAGCACTCTGTTCTCAAA
ACCTGGGGGAATGGAGAAGGCGCATACACCTTAGAGACTGCAGATGCAGAGCAGGACAGGCATTTCTGATGACAG
TCAATTAATGACTTTACAAATTTAAGTCCATCCTAACAAAAGCCCCTT
>SNORD652_Bidirectional_Promoter
(SEQ ID NO: 230)
GATATCTTTTTTTTTTGAAGCGAGTTTTAACAAGATCAGCTGTTTATTCATTCCACTATGGGGTTGAAGGGATCA
TTGGCCAGCTCAAGGCTTACCTTCTCTTGGGCTGAGATGCTGCTGCCAGCTCTAAAACAGCACTCTGTTCTCAAA
ACCTGGGGGAATGGAGAAGGCGCATACACCTTAGAGACTGCAGATGCAGAGCAGGACAGGCATTTCTGATGACAG
TCAATTAATGACTTTACAAATTTAAGTCCATCCTAACAAAAGCCCCTTAAGACCTAATTAGAGGTAATTTTTCTA
AGTTTTTGTAAATTATTGAGGACTACAAATCTTAATTAGCTTCTCAGTAGGTTGTAATTTTTTTTTTTTTTTTGA
GATGGAGTCTCGCTGTTGCCCAGGCTGGAGTGCAGTGGCACGATTTCGACTCACTACAACCTCCGCCTCCCGGGT
TCAAGCGATTCTCCTGGCTCAGCCCCCAAAGTAGCTGGGATTACAAGTACACGCCACCACACCCGGCTAATTTTT
GTATTTTTGGTAGAGATGGGGTTTCACCATGTCGGCCAGCCAGGCTGGTCTTGAACTCCTGACCTCAGGTGATCC
ACCCACCTTAGCCTCCCAAAGTGCTGGGATTACAGGCCACTGTGCCCAGCCTCAGGGGAGTTGTAATCTCCATTT
CAGTCATATCAATTTAAACTTCACAAAGCTAAGATTACTTTTCCTTTTCACATCTGAGGAAAACTACATCTC
>SPDYA1_Bidirectional_Promoter
(SEQ ID NO: 231)
AGGGAGGGGCGGGGTTCGCCGGCGCGCACTCCCAGGCAGGCCCCGCCCCCTCGGCCGGCTGTGCGCGCTGATTGG
CCCCTGCCGGCCTCGCGCTCCCTCGCTCCGGGTTGGCGGGAGACCTTAGAGC
>SPDYA2_Bidirectional_Promoter
(SEQ ID NO: 232)
AGGGAGGGGCGGGGTTCGCCGGCGCGCACTCCCAGGCAGGCCCCGCCCCCTCGGCCGGCTGTGCGCGCTGATTGG
CCCCTGCCGGCCTCGCGCTCCCTCGCTCCGGGTTGGCGGGAGACCTTAGAGCGGGTACCGCTGCTGGCTAGCGAC
CGACGAGCAACCGTCTGAGGCCAGGAGCGCTGCGACGGAGCCTTGACCGCCGTTGCCCGGCCCTCTCCCGCGCAG
CCCCGGGCTTCCGCAG
>SRP_Bidirectional_Promoter
(SEQ ID NO: 233)
GGTCGGATACCGGCGCAGAATAGCACTAGAAGCTGTGGTATGGTGACGTCATCAACTGGGCCAGCCCACAACGCC
TCTAAGATTTCATTTTACTCACCCAGCGAAACAACCTGACCACACTGCGCACGCGTTTCCTTTGAGCACTGCATT
CTGGGTAAACTGTCTCAAAAATTTGAAGAGCGCATGCGTGGGCCAGCTTCTTCCTTTTACCTCGTTGCACTGCTG
AGAGCAAG
>TAF151_Bidirectional_Promoter
(SEQ ID NO: 234)
CTCAGGGTCAGATTCGTGTACGATTTCGTTTTAATGTACCCTTTTCTTCCAGCATCCTTGTTTGCTACTCGGCGA
GACAGTTACAACAAACCGGGAAGCGATCAGGTACGCGAGCTGGTCACGACTCACAGTCCCAGAGCTCGCCGACTC
CGAACGCCCCCAGGTGGCCCAAGCACTCTGCAGCAAAAGCCGCCAGCTAGGACGTACCATTCGAAATTGTAGGGA
AAGAAAGGCTTTGCATAACCAAATACTCTGTGTTTATAAGGTCCCTCCTCTTTCGTTTCCTAACCGCAAATTCCA
TCACACCCAATAAAGTGAGAAATAGGATTGTAAATAAGACGGAGCAAGTAGGTTCCACTTCCTCCCCGATCGTGA
TCGTGGCATTGGTACTTTCTCTTCTCAATTCCCTCTCAATAATGGTACGGCTAGCGGAGGGGGGAATAGAGGGCC
CTGGGAAGGCCTCAGGGCTCGGCGGCTAGTACCAGTGCAGAAACATCCCTCCTGCCGCAGCTTTGTGGTACCACC
CGCTGCCCGCTGATTGGCTGCCGGGGTCCCGC
>TAF152_Bidirectional_Promoter
(SEQ ID NO: 235)
CTCAGGGTCAGATTCGTGTACGATTTCGTTTTAATGTACCCTTTTCTTCCAGCATCCTTGTTTGCTACTCGGCGA
GACAGTTACAACAAACCGGGAAGCGATCAGGTACGCGAGCTGGTCACGACTCACAGTCCCAGAGCTCGCCGACTC
CGAACGCCCCCAGGTGGCCCAAGCACTCTGCAGCAAAAGCCGCCAGCTAGGACGTACCATTCGAAATTGTAGGGA
AAGAAAGGCTTTGCATAACCAAATACTCTGTGTTTATAAGGTCCCTCCTCTTTCGTTTCCTAACCGCAAATTCCA
TCACACCCAATAAAGTGAGAAATAGGATTGTAAATAAGACGGAGCAAGTAGGTTCCACTTCCTCCCCGATCGTGA
TCGTGGCATTGGTACTTTCTCTTCTCAATTCCCTCTCAATAATGGTACGGCTAGCGGAGGGGGGAATAGAGGGCC
CTGGGAAGGCCTCAGGGCTCGGCGGCTAGTACCAGTGCAGAAACATCCCTCCTGCCGCAGCTTTGTGGTACCACC
CGCTGCCCGCTGATTGGCTGCCGGGGTCCCGCAGTCCGCCTCAGCCCGCCGCGCCGCCCTCAGTACAGCTCCGGC
CGCCGCGCCGCCTGGC
>TAF153_Bidirectional_Promoter
(SEQ ID NO: 236)
CTCAGGGTCAGATTCGTGTACGATTTCGTTTTAATGTACCCTTTTCTTCCAGCATCCTTGTTTGCTACTCGGCGA
GACAGTTACAACAAACCGGGAAGCGATCAGGTACGCGAGCTGGTCACGACTCACAGTCCCAGAGCTCGCCGACTC
CGAACGCCCCCAGGTGGCCCAAGCACTCTGCAGCAAAAGCCGCCAGCTAGGACGTACCATTCGAAATTGTAGGGA
AAGAAAGGCTTTGCATAACCAAATACTCTGTGTTTATAAGGTCCCTCCTCTTTCGTTTCCTAACCGCAAATTCCA
TCACACCCAATAAAGTGAGAAATAGGATTGTAAATAAGACGGAGCAAGTAGGTTCCACTTCCTCCCCGATCGTGA
TCGTGGCATTGGTACTTTCTCTTCTCAATTCCCTCTCAATAATGGTACGGCTAGCGGAGGGGGGAATAGAGGGCC
CTGGGAAGGCCTCAGGGCTCGGCGGCTAGTACCAGTGCAGAAACATCCCTCCTGCCGCAGCTTTGTGGTACCACC
CGCTGCCCGCTGATTGGCTGCCGGGGTCCCGCAGTCCGCCTCAGCCCGCCGCGCCGCCCTCAGTACAGCTCCGGC
CGCCGCGCCGCCTGGCTTTCGTATTCGTTGTTCTCGGCGGGCTGTGGGGCCTCCGCGCCGCGGCCGTTAGTC
>TBL31_Bidirectional_Promoter
(SEQ ID NO: 237)
CGAAGCACCCTCACAGCTCACGGCCCTCCCTCCAGGCCGGAAACGTCTCCGCCCGCTTCCGCTTCCCGATGCAGC
CGCCACTGCCCGAAGCAAAGATGGCGCCAAGTGCGCGGCGCCGGGGGGACGTCACAGTGGTCGCGCGCGGTGAC
GCCATCGCAGCGCGCC
>TBL32_Bidirectional_Promoter
(SEQ ID NO: 238)
CGAAGCACCCTCACAGCTCACGGCCCTCCCTCCAGGCCGGAAACGTCTCCGCCCGCTTCCGCTTCCCGATGCAGC
CGCCACTGCCCGAAGCAAAGATGGCGCCAAGTGCGCGGCGCCGGCGGGGACGTCACAGTGGTCGCGCGCGGTGAC
GCCATCGCAGCGCGCCGGGAGTGTGGCGTTCTGTGAAGAGTTCGGTGCTAACCTCCCTCACGCGGCGGTGGCTGC
CGGGACCCTAGCAGGTTTCAGCTGGAGCGGCGGCGGCGGCAAC
>ZFY1_Bidirectional_Promoter
(SEQ ID NO: 239)
TTTTTTTAAAGCCAACAAAGGAGACAGTGGGGAATGCTATATGTCTGTATCTGCTTTCCTCCTCAACCCTAGGAA
TAAAGTAAACACGTTTACTGAGGGCGGGGGTCTAAGGGCCTGCAACAATGAGATCTGTCGCCTTGGCTAGGACTG
GCGCCGAGAGGCGATAGGTCTCGGGAGAGCCTGGCGCAGGGTGTGGGAGATTAGGAATCCCAGGTCCACCGGAGA
TGGCAGGGGGTGGCCTGGCCCGGTGCGGGGCCGCTTGCCTGCACGCAACCAACTAAGGCGGTGGTGCGCAAGT
>ZFY2_Bidirectional_Promoter
(SEQ ID NO: 240)
TTTTTTTAAAGCCAACAAAGGAGACAGTGGGGAATGCTATATGTCTGTATCTGCTTTCCTCCTCAACCCTAGGAA
TAAAGTAAACACGTTTACTGAGGGGGGGGGTCTAAGGGCCTGCAACAATGAGATCTGTCGCCTTGGCTAGGACTG
GCGCCGAGAGGCGATAGGTCTCGGGAGAGCCTGGCGCAGGGTGTGGGAGATTAGGAATCCCAGGTCCACCGGAGA
TGGCAGGGGGTGGCCTGGCCCGGTGCGGGGCCGCTTGCCTGCACGCAACCAACTAAGGCGGTGGTGCGCAAGTAG
TGGTGACGGCGGGCGCGCGGAGAAAAGGAACGTTGTGACGGAAACTCCAGCTGCCGGAGACCCCACCGCAGTGAG
GTCACTGGACTCCCCGGACTCGGGGCGTGACCGGCGCCGACCCGGGGCGCCGAGAGGCCCACCGGGCGGAGGGGG
CCCAACTACCATCCCGCATTTTCCTGGGTCTCTCTCCCGGGCGGTGACGTGACGTGCTGACGGCGGGCCCGTGCC
GGGGAGCTGGGCCGCTTTTTGTCAGCTCCGAACTCGGCCCCTCCTCCCTCCCTCCGCCCGCCCTACCAGCCGGAG
CCCGGCCCAGTGCTCCAGAGAAAGGCCGTCCTGCAGCACCCGCCGCTGTCGCCGACCGCCCGCACATCCGTCGGG
TGAGTCCCGCGTGCCCCCGCGGCCGCGGG
>SRP-RPS29
(SEQ ID NO: 241)
CTTGCTCTCAGCAGTGCAACGAGGTAAAAGGAAGAAGCTGGCCCACGCATGCGCTCTTCAAATTTTTGAGACAGT
TTACCCAGAATGCAGTGCTCAAAGGAAACGCGTGCGCAGTGTGGTCAGGTTGTTTCGCTGGGTGAGTAAAATGAA
ATCTTAGAGGCGTTGTGGGCTGGCCCAGTTGATGACGTCACCATACCACAGCTTCTAGTGCTATTCTGCGCCGGT
ATCCGACC
>7skl_Bidirectional_Promoter
(SEQ ID NO: 242)
GAGGTACCCAAGCGGCGCACAAGCTATATAAACCTGAAGGAAGTCTCAACTTTACACTTAGGTCAAGTTGCTTAT
CGTACTAGAGCTTCAGCAGGAAATTTAACTAAAATCTAATTTAACCAGCATAGCAAATATCATTTATTCCCAAAA
TGCTAAAGTTTGAGATAAACGGACTTGATTTCCGGCTGTTTTGACACTATCCAGAATGCCTTGCAGATGGGTGGG
GCATGCTAAATACT
>7Sk2_Bidirectional_Promoter
(SEQ ID NO: 243)
GAGGTACCCAAGCGGCGCACAAGCTATATAAACCTGAAGGAAGTCTCAACTTTACACTTAGGTCAAGTTGCTTAT
CGTACTAGAGCTTCAGCAGGAAATTTAACTAAAATCTAATTTAACCAGCATAGCAAATATCATTTATTCCCAAAA
TGCTAAAGTTTGAGATAAACGGACTTGATTTCCGGCTGTTTTGACACTATCCAGAATGCCTTGCAGATGGGTGGG
GCATGCTAAATACTGCAGTCTCCATTGGTGAGGTCGTCCCGGAGCCTCGCCCAGCTCCCGCGCGCTAGAGCCGCC
TGCTGGTCTCACCCAGCCGGGACCGCTGACCTGGCGCTTTGTGCGGCTCCAGGCCTCCGAGTGGACTCCAGAAAG
CCTGAAAAGCTATC
>7sk3_Bidirectional_Promoter
(SEQ ID NO: 244)
GAGGTACCCAAGCGGCGCACAAGCTATATAAACCTGAAGGAAGTCTCAACTTTACACTTAGGTCAAGTTGCTTAT
CGTACTAGAGCTTCAGCAGGAAATTTAACTAAAATCTAATTTAACCAGCATAGCAAATATCATTTATTCCCAAAA
TGCTAAAGTTTGAGATAAACGGACTTGATTTCCGGCTGTTTTGACACTATCCAGAATGCCTTGCAGATGGGTGGG
GCATGCTAAATACTGCAGTCTCCATTGGTGAGGTCGTCCCGGAGCCTCGCCCAGCTCCCGCGCGCTAGAGCCGCC
TGCTGGTCTCACCCAGCCGGGACCGCTGACCTGGCGCTTTGTGCGGCTCCAGGCCTCCGAGTGGACTCCAG
>_RMRP-CCDC107
(SEQ ID NO: 245)
TGCCGGCCCACGGGTGGAGGGATCGGGCGGGCGGTGCCGAAGCGGTCCGGCATTGGCCGGCCGCCCCAACGCGCA
CGCGCACGCGAGCAGGCCGGCCGGCTCCGGGGAGGCCACGCCCACTCCCCGTAGGGGGGGGCCAGACCATATTTG
CATAAGATAGTGTCATTCTAGCTTTCCTGTATTTGTTCATTTCGTGTCTATTAGCTATTCTGCTAGCCACAATGC
CTCTGAAAGCCTATAGTCTTAGAAAGTTATGCCCGAAAACGGTTTTTTTAATCTCACGCCACCAACTTTCTCACC
CTAATCATAAAACACAATTTCTTTAGGGCTATAAAATACTACTCTGTGAAGCTGAGGACGT
>ALOXE3_Bidirectional_Promoter
(SEQ ID NO: 246)
TCTTCACGAGAGCTTTACTTTTTGCTTATAAGAGGGTTCTCTATAGGAAAAGCCAGGCTTGTAGAACCGACAGAG
GATTTTATCTGTGCAGCATAGAATATTTTGGCACAGATTTGGAAGCAGCGGGTGAAGCTCGCCTGCTGCTGATTG
AGCTTTTTCTGCCTCCCGTTCTTAGAGCCCCCGCCGAGGCTGCGACGCAGGGACTGTACCATAGTAGAGGCTGGA
ACAGTGCGGCGCCGGAACCGGCCGCGCGGGGCCGCTGCGGGCTATGGGCTTCTCTGAGAGGTTCCTCCCCAGTCC
CTAGTGGCCCAGATCCCGGACACCTGGGCTCCCGCCCAGGATCCTGCAGGCCCAGGGCGGTCCTGGAGCGGAAAG
A
>CGB1_Bidirectional_Promoter
(SEQ ID NO: 247)
TTGTCGGGCCCATCCTTTCTTCCCTTTGATCTTACGCAGGGTGATGGAGCCAATCACAAGAGGCTCATCCCTGAC
GTCACCCAGTCCCCAGGGCCAGTGAGGGCCCTGCGTTCCGTGGCGCCCCCTGGAGGGAGGAAGGGGAACTGCATC
TGAGAGAGAGCAGCCAATTGGGTCCGCTGACTCTGGCCAGGTTCCCGTGCCGCGTCCAACACCCCTCACTCCCTG
TCTCACTCCCCCACGGAGACTCAATTTACTTTCCATGTCCACATTCCCAGTGCTTGCGGAAGATATCCCGCTAAG
AGAGAGAC
>CGB2_Bidirectional_Promoter
(SEQ ID NO: 248)
GTGTCGGGGATCTCCTTTCTTCCTTTTGACCTTACGCAGGGTGATGGAGCCAATCAGGAGAGGCTCACCCCTGAC
GTCACCCAGTCCCCAGGGCCAGTGAGGGCCCTGCGTTCCGTGGCGCCCCCTGGAGGGAGGAAGGGGAACTGTATC
TGAGAGAGAGCAGCCAATTGGGTCCGCTGACTCCGGCCGGGTTCCCGTGCCGCGTCCAACACCCCTCACTCCCTG
TCTCACTCCCCCACGGAGACTCAATTTACTTTCCATGTCCACATCCCCAGTGCTTGCGGAAGATATCCCGCTAAG
AGAGAGAC
>Med16-1_Bidirectional_Promoter
(SEQ ID NO: 249)
GAATATTGAGTTCCACCACCAGCTATTTAAAGCCCCTGGAACAAATGTCTGTACACATAGGCCGACTTCTCTTAA
ATGACCTAGAGATTTAACCTCTATTTATATTAGCCCAATGTGTAATGCAACTAACGTAGTTATTGACTGGAGTTG
AGAAAGTGCTCGTTGTTCTACCAAATATAGCTACGGTGGCTGCTGGGAATTACTGGAAATGGTCGTATGCAAATA
GCCCCGGAGGCGGGGCAGAGCCTGAGCCGCACCGCCCTCCCAGAAGTCTTTGGGAGGCGGCCCCACGCCTCAGGC
GACTGGTTGTTACCGAGGAAGATGGCGGCGCCAGACCCGAGGCGCTAGGGAAGATCGCACCGCGGACGCCCGCTG
AGCTTGGCGCACGGGCCAGGAGCTGGTGACTGCCCTC
>Med16-2_Bidirectional_Promoter
(SEQ ID NO: 250)
GAATATTGAGTTCCACCACCAGCTATTTAAAGCCCCTGGAACAAATGTCTGTACACATAGGCCGACTTCTCTTAA
ATGACCTAGAGATTTAACCTCTATTTATATTAGCCCAATGTGTAATGCAACTAACGTAGTTATTGACTGGAGTTG
AGAAAGTGCTCGTTGTTCTACCAAATATAGCTACGGTGGCTGCTGGGAATTACTGGAAATGGTCGTATGCAAATA
GCCCCGGAGGCGGGGCAGAGCCTGAGCCGCACCGCCCTCCCAGAAGTCTTTGGGAGGCGGCCCCACGCCTCAGGC
GACTGGTTGTTACCGAGGAAGATGGCGGCGCCAGACCCGAGGCGCTAGGGAAGATCGCACCGCGGACGCCCGCTG
AGCTTGGCGCACGGGC
>DPP9-1_Bidirectional_Promoter
(SEQ ID NO: 251)
CCTGATAGGTAGCATCCTCTCCGGATATCCTTAATAGTGGGGGATCATGGGTTTGACTGAGTGATACCAAGTCAC
AGGGGGGTGTCTCTCCCTAACCCACCGGAAGATGTCGTTCATGGGGCGTTACGCACCTTAGGCCGCCGCGCCGCG
GGCTCCCCCCCAAGCGCCGCGGACGCCTTGGTACGTGCCTGGTGGTGTCCAATCCCAGGCCGCCGCCTGGGTCGC
TCAACTTCCGGGTCAAAGGTGCCTGAGCCGGCGGGTCCCCTGTGTCCGCCGCGGCTGTCGTCCCCCGCTCCCGCC
ACTTCCGGGGTCGCAGTCCCGGGCATGGAGCCGCGACCGTGAGGCGCCGCTGGACCCGGGACGACCTGCCCAGTC
CGGCCGCCGCCCCACGTCCCGGTCTGTGTCCCACGCCTGCAGCTGGAATGGAGGCTCTCTGGACCCTTTAGAAGG
CACCCCTGCCCTCCTGAGGTCAGCTGAGCGGTTA
>DPP9-2_Bidirectional_Promoter
(SEQ ID NO: 252)
CCTGATAGGTAGCATCCTCTCCGGATATCCTTAATAGTGGGGGATCATGGGTTTGACTGAGTGATACCAAGTCAC
AGGGGGGTGTCTCTCCCTAACCCACCGGAAGATGTCGTTCATGGGGCGTTACGCACCTTAGGCCGCCGCGCCGCG
GGCTCCCCCCCAAGCGCCGCGGACGCCTTGGTACGTGCCTGGTGGTGTCCAATCCCAGGCCGCCGCCTGGGTCGC
TCAACTTCCGGGTCAAAGGTGCCTGAGCCGGCGGGTCCCCTGTGTCCGCCGCGGCTGTCGTCCCCCGCTCCCGCC
ACTTCCGGGGTCGCAGTCCCGGGCATGGAGCCGCGACCGTGAGGCGCCGCTGGACCCGGGACGACCTGCCCAGTC
CGGCCGCCGCCCCACGTCCCGGTCTGTGTCCCACGCCTGCAGCTGGAATGGAGGCTCTCTGGACCCTTTAGAAG
>DPP9-3_Bidirectional_Promoter
(SEQ ID NO: 253)
CCTGATAGGTAGCATCCTCTCCGGATATCCTTAATAGTGGGGGATCATGGGTTTGACTGAGTGATACCAAGTCAC
AGGGGGGTGTCTCTCCCTAACCCACCGGAAGATGTCGTTCATGGGGCGTTACGCACCTTAGGCCGCCGCGCCGCG
GGCTCCCCCCCAAGCGCCGCGGACGCCTTGGTACGTGCCTGGTGGTGTCCAATCCCAGGCCGCCGCCTGGGTCGC
TCAACTTCCGGGTCAAAGGTGCCTGAGCCGGCGGGTCCCCTGTGTCCGCCGCGGCTGTCGTCCCCCGCTCCCGCC
ACTTCCGGGGTCGCAGTCCCGGGCATGGAGCCGCGACCGTGAGGCGCCGCTGGACCCGGGACGACCTGCCCAGTC
CGGCCGCCGCCCCACGTCCCG
>SNORD13_C8orf41
(SEQ ID NO: 254)
TCCTGACTGCAGCACCAGAAGGCTGGTCTCTCCCACAGAACGAGGATGGAGGGGGGAGGGATCCGTTGAAGAGG
GAAGGAGCGATCACCCAAAGAGAACTAAAATCAAATAAAATAAAACAGAGAGATGTCTTGGAGGAGGGGGCGAGT
CTGACCGGGATAAGAATAAAGAGAAAGGGTGAACCCGGGAGGCGGAGTTTGCAGTGAGCCGAGATCGCGCCACTG
CACTCCAGCCTGGGCGACAGAGTGAGACTCCGTCTCAGTAAAAAAAAAAAAAAAAAAAAGAATAAAGAGGAAAGG
ACGCAAGAAAGGGAAAGGGGACTCTCAGGGAGTAAAAGAGTCTTACACTTTTAACAGTGACGTTAAAAGACTACT
GTTGCCTTTCTGAAGACTAAAAAGAAAAAAAACTTAAAAATTTAAAGAAATAAACTTCTGAGCCATGTCACCAAC
TTAACCACCCCCAGGTACCTGCAACGGCTCGCGCCCGCCGGTGTCTAACAGGATCCGGACCTAGCTCATATTGCT
GCCGCAAAACGCAAGGCTAGCTTCCGCCAGTACTGCCGCAACACCTTCTTATTTCACGACGTATGGTCGTAAAGC
AATAAAGATCCAGGCTCGGGAAAATGACGGAGAGGTGGAACTATAGAGAATAAATTTGCATATATAATAATCCGC
TCGCTAATTGTGTTTCTGTTTTCCTTTGCTAAGGTAGAAACAAAAGAATAATCACAGAATCTCAGTGGGACTTTG
AAAATATCCAGGATTTTATACGTGAAGAATGGATGTATCGCATTACGGTAGTCACCCTATGTGTAAATTAGTGGC
ACATACTTGGCACTCCTTAATGTCAACTATAAGATG
>THEM259_Bidirectional_Promoter
(SEQ ID NO: 255)
GACTCAAGGGTTACTGTCACACCTATTTTAAGCCCTTCAATCAAATCATCTTTTGGTTAGGATAACTTATGGTCG
GTTTCATATTTAGCATAATTTCCTACAGTGGTATGTTGCAGAACAACTTTCGTGCTTACGCTTACTTTGATGTCT
TCGATCACGTAAAATCCCATATCTTATCGTAATTTTACCGCCTTATACTGGCCTCATAGCCGCGGTGGATTGTGG
GTGCCAATATGCAAAAGAGGTGGCCCAGATGCAGGCCCGCCCCCTGGAGCGGCCGAGGTAGGGGGTGAGGCCTCC
GCGGGCGCCGCTGGCATCCCAGCGTTCTCTGCGGGCGCAGGGGGGCCGCTCTTGCCCGGCGTGGCGACTCGCTAG
CGTCAGCAGCGCCGCAGCCGGACGAGAAAGCGGAAGATGGCGGCGGCGGCCGGGAGGCCGTGAGGAGAGCGGCGG
CTGCGAGGGCGGCCGATGGCGGCCGGGAGGCGCCCTCGGACACTTGCGGGTCGTTAGGGCGCGACGCTGGGAGGC
>H1_2-H1_83
(SEQ ID NO: 936)
TGGCAAACACCGCCGGCGCAAACAGCATTTATAATGCGCTCATACCTAAAGCCACTTGTCGGTTACGGTGACTTC
CCAACAAGACATTGCGACATGCAAATACTACAGTGCGTCCCGCCCCCTGGTGTAGTTCCACGCTGGGACGCACAC
GCACTACGGTTCCCGCCTTTAGACGACTGCGCTGGCGATTCCTGGGAGAGGACTGATGACGTCAGCGTTCGGGCT
CC
>H1_2-H1_90
(SEQ ID NO: 937)
TGGCAAACACTGCCGGCTCAAACAGCATTTATAATGCGCTCATACCTAAAGCCACTTGTCGGTTACGGTGACTTC
CCAACAAGACATTGCGACATGCAAATACTGCGGTGCGTCCCGCCCCCTGGTGTAGTTCCACGCTGGGACGCACAC
GCACTACGGTTCCCGCCTTTAGACGACTGCGCCGGCGATTCCTGGGAGAGGACTGATGACGTCAGCGTTCGGGCT
CC
>H1_2-H1_92
(SEQ ID NO: 938)
TGGCAAACAACGCCGGCGCAAACAGCATTTATAATGCGCTCATACCTAAAGCCACTTGTCGGTTACGGTGACTTC
CCAACAAGACATTGCGACATGCAAATATTACAGTGCGTCCCGCCCCCTGGTGTAGTTCCACGCTAGGACGCACAC
GCACTACGGTTCCCGCCTTTAGACGACTGCGCTGGCGATTCCTGGGAGCGGACTGATGACGTCAGCGTTCGGGCT
CC
>H1_2-H1_95
(SEQ ID NO: 939)
TGGCAAAAACTGACGGCTCAAGCAGCATTTATAAGGCTCCCATACCTAAAGACATTTGTCGGTTATGGTGACTTC
CCCACAAGACATTGCGACATGCAAATACTGCGGAGCGTACCCCCTCCCCCTGGCGCAACTCCTCGCTGGGACGCA
CGCGCGCTACGTGTTCCCGCCTTTAGTGACGTCTGCGCCGGCGATTCCTGGGAGAGGGTTGATGACGTCAGCGTT
CGGGCTCC
>H1_2-H1_98
(SEQ ID NO: 940)
TGGGAAAAAGTGGCGGCTCACGCAGCATTTATAAGGCTCCCATACCTAAAGACATTTCACGGTTATGGTGACTTC
CCCACAAGACATAGCGACATGCAAATATTGCGGAGCGTACGCGCCTCCCCCTGTCCTGTGCAGGCATCTTCTCAG
CCAGGACGCACGCGCGCTGCGTGTTCCCGCCCTGAGTGACTTCTGCGCCGGCGATTTCCTGGGAGGAGGGTTGAT
GACGTCAACGTTCGGGCTCC
>H1_2-H1_104
(SEQ ID NO: 941)
TGGCAAAAACTGCCGGCTCAAGCAGCATTTATAATGCGCCCATACCTAAAGCCACTTGTCGGTTACGGTGACTTC
CCAACAAGACATTGCGACATGCAAATACTGCGGTGCGTCCCTCCCCCTGGCGTAACTCCACGCTGGGACGCACGC
GCGCTACGTGTTCCCGCCTTTACTGACGTCTGCGCCGGCGATTCCTGGGAGAGGGTTGATGACGTCAGCGTTCGG
GCTCC
>H1_2-H1_113
(SEQ ID NO: 942)
TGGGAAAAAGTGGCGGCTCACGCAGCATTTATAAGGCTCCCATACCTAAAGACATTTCACGGTTATGGTGACTTC
CCCACAAGACATTGCGACATGCAAATATTGCGGAGCGTACGCCCTCCCCCTGTCCTGTGCAGGCATCTTCTCGCC
AGGACGCACGCGCGCTGCGTGTTCCCGCCTTGAGTGACTTCTGCGCCGGCGATTTCCTGGGAGGAGGGTTGATGA
CGTCAACGTTCGGGCTCC
>H1_2-H1_188
(SEQ ID NO: 943)
TGGGAAAAAGTGGGGGCTCACGCAGCATTTATAAGGCTCCCATACCTAAAGACATTTCACGGTTATGGTGACTTC
CCCCACAATACATAGCGACATGCAAATATCATGGTCCTTCCGCGGGGCGTGCGGCCTCCCCCTGTCCCGTGCCCC
GTAGGCGTCTTCTCAGCCAGGAGACGCACGCGGCGCGCTGCGTGTTCCCGCCCTGAGTGACTTCTGGGCCGGCGA
TTTCCCTGGGAGGAGGGTTGGATGACGTCAGCATCGCCAACGTTCGGGCTCC
>H1_2-H1_189
(SEQ ID NO: 944)
TGGGAAAAAGTGGGGCTCACGCAGAATTTATAAGGCTCCCATACCTAAAGACATTTCACGGTTATGGTGACTTCC
CCCACAATACATAGCGACATGCAAATATCATGGTCCTTCCGCGGGGCGTGCGGCCTCCCCCTGTCCCGTACCCCG
CAGGCGTCTTCTCAGCCAGGAGGCGCACGCGGCGCGCTGCGCCCTGTTCCCGCCCTGAGTGACTAGGGATTCTGG
GCCCGCGATTTCCCGCTGGGAGGCGGGTTGGATGACGTCAGCATCGCCAGCGTTCGGGCTCC
>H1_2-H1_241
(SEQ ID NO: 945)
TGGGAAAAAGTGGGGGCTCACGCAGCATTTATAAGGCTCCCATACCTAAAGACATTTCACGGTTATGGTGACTTC
CCCACAATACATAGCGACATGCAAATATCGCGGGGCGTGCGGCCTCCCCCTGTCCCGTGTAGGCGTCTTCTCAGC
CAGGACGCACGCGCGCTGCGTGTTCCCGCCCTGAGTGACTTCTGGGCCGGCGATTTCCCTGGGAGGAGGGTTGAT
GACGTCATCGCCAACGTTCGGGCTCC
>H1_2-H1_301
(SEQ ID NO: 946)
TGGGAAAAAGTGGGGCTCACGCAGAATTTATAAGGCTCCCATACCTAAAGACATTTCACGGTTATGGTGACTTCC
CCCACAATACATAGCGACATGCAAATATCATGGTCCTTCCGCGGGGCGTGCGGCCTCCCCCTGTCCCGTACCCCG
CAGGCGTCTTCTCAGCCAGGAGGCGCACGCGGCGCGCTGCGCCCTGTTCCCGCCCTGAGTGACTAGGGATTCTGG
GCCCGCGATTTCCCTGGGAGGCGGGTTGGATGACGTCAGCATCGCCAGCGTTCGGGCTCC
>H1_2-H1_306
(SEQ ID NO: 947)
TGGGAAAAAGTGGGGGCTCACGCAGAATTTATAAGGCTCCCATACCTAAAGACATTTCACGGTTATGGTGACTTC
CCCCACAATACATAGCGACATGCAAATATCATGGTCCTTCCGCGGGGCGTGCGGCCTCCCCCTGTCCCGTACCCC
GTAGGCGTCTTCTCAGCCAGGAGACGCACGCGGCGCGCTGCGCCCTGTTCCCGCCCTGAGTGACTAGGGATTCTG
GGCCGGCGATTTCCCTGGGAGGCGGGTTGGATGACGTCAGCATCGCCAGCGTTCGGGCTCC
>H1_2-H1_312
(SEQ ID NO: 948)
TGGGGAAAGGTGGGCTCAAGCAGAATTTATAAGGCTCCCAAAACTAAAGACATTTTTCGGTTATGGTGACTTCCC
CCACAATACACAGCGACATGCAAATATCATGGCCCTTCCGTGGAGTGTGCCCTCCCTGCGCTCGTCCCCCGGGCC
TCTTCTCAGCCAGGAGGCGCACGGCGCGCTGCGCCTGTTCCCGCCCTGGGGACTAGGAGCGCGCCCGCGGTTCCC
GCTGGGAGGCGGGTTGGATGACGTCAGCATCGCCAGCGTTCGGACTCC
>H1_2-H1_352
(SEQ ID NO: 949)
TGGGGAGTGGGGGGCTCAGGCCGAATTTATAAGGCTCCCAAAACGGAAGACATTTTTCAGTTATGGTGACTTCCC
CCACAAGACACAGCGCTATGCAAATATCATGGCCCCTCCGTGGAGTGTGCCCTCCCCGGCCGCTTCTCAGCCAGG
AAGCGCACGGCGCGTCTGCGCCTGTTTCCCGCCCTGGGGACTAGAAAAGCGCCCGCGCATCCCGGCCGGGCCGCG
GGTTGATGACGTCAGCATCGCCAGCGCTCGAGCGCC
>H1_2-H1_370
(SEQ ID NO: 950)
TGGGGAAAGGTGGGCTCAAGCAGAATTTATAAGGCTCCCAAACCTAAAGACATTTTACGGTTATGGTGACTTCCC
CCACAACACACAGCGACATGCAAATATCATGGTCCTTCCGTGGAGTGTGCCCTCCCTGCGCTCGTCCCCCGGGCC
TCTTCTCAGCCAGGAGGCGCACGCGCGCACGCGCGCTGCGCCTGTTCCCGCCCTGGTGACTAGGAGCGCGCCCGC
GGTTCCCGCTGGGAGGCGGGTTGGATGACGTCAGCATCGCCAGCGTTTGGACTCC
>H1_2-H1_398
(SEQ ID NO: 951)
TGGGAAAAAGTGGGGCTCAAGCAGAATTTATAAGGCTCCCAAACCTAAAGACATTTTACGGTTATGGTGACTTCC
CCCACAACACACAGCGACATGCAAATATCATGGTCCTTCCGCGGGGTGTGCGGCCTCCCTGCTCTCGTCCCCCAG
GCGTCTTCTCAGCCAGGAGGCGCACGCGCGCACGCGCGCTGCGCCCTGTTCCCGCCCTGGTGACTAGGGAGCCTG
AGCCCGCGATTTCCCGCTGGGAGGCGGGTTGGATGACGTCAGCATCGCCAGCGTTTGGACTCC
>H1_2-H1_401
(SEQ ID NO: 952)
TGGGGAGTGGGGGGCTCAGGCCGAATTTATAAGGCTCCCAAAACGGAAGACATTTTTCAGTTATGGTGACTTCCC
CCACAAGACACAGCGCTATGCAAATATCATGGCCCCTCCGTGGAGTGTGCCCTGGCCCCGGCCGCTTCTCAGCCA
GGAAGCGCACGGCGCGCTGCGCCTGTTCCCGCCCTGGGGACTAGAAAAGCGCCCGCGCATCCCGCCGGGCCGCGG
GTTGGATGACGTCAGCATCGCCAGCGCTCGAGCGCC
>H1_2-H1_402
(SEQ ID NO: 953)
TGGGGAGTGGCGGCCTCAGGCGGGATTTATAAGGCTCCCAAAACCGGTGCCATTTCTCAGTGAGGGTGACTTCCC
CCACAATACACAGCGGTATGCAAATATCAGTTGCGTCAGAGTAGAGCGCGGCCTCCCCGGCCTCTCCTCAGCCAG
GAAGCGCGCGGCGCTCCTGTTTTCGTCTCCCGCCCCGGTGACGAGAGACGCGCGCGCGCACCGTAGCCGGGCCGC
GGGTTGGTGACGTAAGCGGCATCCGCTTTCGAGCGCC
>H1_14-H1_18
(SEQ ID NO: 954)
CGGCAAATAACGCGCGCAAACAGCATTTATAATGAGCTCATACCTAAAGCCACTTTACGGTTACGGTGACTTCCC
ACAAGACATTGCGGCATGCAAATATTTTAGTGCGTCCCGCCCCTGGTAGTTCCACGCTAGGACGCACACGCACTA
CGGTTCCCGCCTTTAGACTGCGCTGGCGATTCCAGCGAGCGGACTGATGACGTCAGCGTTGGGGCTCC
>H1_16-H1_17
(SEQ ID NO: 955)
CGGCGAACAACGCGCGCAAACAGCATTTATAATGCGCTCATACCTAAAGCCACTTTTCGGTTACGGTGACTTCCC
ACAAGACATTGCGGCATGCAAATATTTTAGTGCGTCCCGCCCCTGGTAGTTCCACGCTAGGACGCACACGCACTA
CGGTTCCCGCCTTTAGACTGCGCTGGCGATTCCAGCGAGCGGACTGATGACGTCAGCGTTGGGGCTCC
>H1_21-H1_27
(SEQ ID NO: 956)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTC
CGGTTCCCGCCTTTAGACTGCGCTGGCGATTCCAGGGAGCGGGCTGATGACGTCAGTGTTCGGGCTCC
>H1_23-H1_21
(SEQ ID NO: 957)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTC
CGGTTCCCGCCTTTAGACTGCGCTGGCGATTCCAGGGAGCGGACTGATGACGTCAGTGTTCGGGCTCC
>H1_23-H1_24
(SEQ ID NO: 958)
CGGCCAACAGCTCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTG
CGGTTCCCGCCTTTAGACTGCGCTGGCGATTCCAGGGAGCGGACTGATGACGTCAGTGTTCGGGCTCC
>H1_25-H1_26
(SEQ ID NO: 959)
CGGCAAACAATGCGCGCAAACAGCATTTATAATGAGCTCATACCTAAAGCCACTTTACGGTTACGGTGACTTCCC
ACAAGACATTGCGATATGTAAATATTTTAGTGCATCCCGCCCCTGGTAGTTCCACGCTAGGACGCACACGCACTA
CGGTTCCCGCCTTTAGATTGCGCTGGCGATTCCAGGGAGCGGACTGATGACGTCAGCGTTCGGGCTCC
>H1_27-H1_28
(SEQ ID NO: 960)
CGGCAAACAACGCGCGCAAACAGCATTTATAATGAGCTCATACCTAAAGCAACTTTTCGGTTACGGTGACTTCCC
ACAAGCCATTGCGACATGCAAATATTTTAGTGTATCCCGCCCCTGGTAGTTCCACGCTAGGACGCACACGCACTC
CGGTTCCCGCCTTTAGACTGCGCTGGCGATTCCGGGGAGCGGGCTGATGACGTCAGTGTTCGGGCTCC
>H1_31-H1_33
(SEQ ID NO: 961)
CGGCAAACAATGCGTGCACACAGCACTTATAATGCGCTCACACCTAAAGCCACTTTTCAGTTACGGTGACTTCCC
ACAAGACATTGCGATATGCAAATATTTTAGCGCATCCCGCCCCTGGTAGTTCCACGCGAGGACGCACACGCACTA
CGGTTCCCGCCTTTAGACTGCGCTGGCGATTCCAGGGAGCGGCCTGATGACGTCAGCGTTCGGGCTCC
>H1_34-H1_32
(SEQ ID NO: 962)
CGGCAAACAATGCGTGCACACAGCATTTATAATGCGCTCACACCTAAAGCCACTTTTCAGTTACGGTGACTTCCC
ACAAGACATTGCGATATGCAAATATTTTAGCGCGTCCCGCCCCTGGTAGTTCCACGCGAGGACGCACACGCACTA
CGGTTCCCGCCTTTAGACTGCGCTGGCGATTCCAGGGAGCGGCCTGATGACGTCAGCGTTCGGGCTCC
>H1_35-H1_37
(SEQ ID NO: 963)
CGGCAAACAGTGCGCGCAAACAGCATTTATAATGCGCTCATACCTAAAGCCACTTTTCGGTTACGGTGACTTCCC
ACAAGACATTGCGACATGCAAATATTTTAGTGCGTCCCGCCCCTGGTAGTTCCACGCTAGGACGCACACGCACTA
CGGTTCCCGCCTTTAGACTGCGCTGGCGATTCCTGGGAGCGGACTGATGACGTCAGCGTTCGGGCTCC
>H1_36-H1_20
(SEQ ID NO: 964)
CGGCAAACAACGCGCGCAAACAGCATTTATAATGCGCTCATACCTAAAGCCACTTTACGGTTACGGTGACTTCCC
ACAAGACATTGCGACATGCAAATATTTTAGTGCATCCCGCCCCTGGTAGTTCCACGCTAGGACGCACACGCACTA
CGGTTCCCGCCTTTAGACTGCGCTGGCGATTCCAGGGAGCGGACTGATGACGTCAGCGTTCGGGCTCC
>H1_39-H1_22
(SEQ ID NO: 965)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTA
CGGTTCCCGCCTTTAGACTGCGCTGGCGATTCCAGGGAGCGGACTGATGACGTCAGTGTTCGGGCTCC
>H1_39-H1_89
(SEQ ID NO: 966)
CGGCAAACAACGCGCGCAAACAGCATTTATAATGAGCTCATACCTAAAGCAACTTTACGGTTACGGTGACTTCCC
ACAAGACATTGCGACATGCAAATATTTTAGTGTATCCCGCCCCTGGTAGTTCCACGCTAGGACGCACACGCACTA
CGGTTCCCGCCTTTAGACTGCGCTGGCGATTCCAGGGAGCGGGCTGATGACGTCAGCGCCCGGGCTCC
>H1_41-H1_40
(SEQ ID NO: 967)
TGGCAAACAATCCGCGCAAACAGCATTTATAATGCGCTCATACCTAGAGCCACTTTTCGGTTACGGTGACTTCTC
ACAAGACATTGGGACATGCAAATATTACAGTGCGTCCCGCCCCTGGTTAGTTCTACGCTAGGACGCACACGCACT
ACGGTTCCCGCCTTTAGACTGCGCTGGCGGTTCCTGGGAGCGGACTGATGACGTCAGTGTTCGGGATCC
>H1_41-H1_55
(SEQ ID NO: 968)
TGGCAAACAACGCGCGCAAACAGCATTTATAATGCGCTCATACCTAGAGCCACTTTTCGGTTACGGTGACTTCTC
ACAAGACATTGGGACATGCAAATATTACAGTGCGTCCCGCCCCTGGTTAGTTCTACGCTAGGACGCACACGCACT
ACGGTTCCCGCCTTTAGACTGCGCTGGCGATTCCTGGGAGCGGACTGATGACGTCAGCGTTCGGGATCC
>H1_47-H1_41
(SEQ ID NO: 969)
TGGCAAACAACGCCGGCGCAAACAGCATTTATAATGCGCTCATACCTAGAGCCACTTTTCGGTTACGGTGACTTC
TCAACAAGACATTGGGACATGCAAATATTACAGTGCGTCCCGCCCCTGGTGTAGTTCTACGCTAGGACGCACACG
CACTACGGTTCCCGCCTTTAGACGACTGCGCTGGCGATTCCTGGGAGCGGACTGATGACGTCAGCGTTCGGGATC
C
>H1_47-H1_43
(SEQ ID NO: 970)
TGGCAAACACCGCACGCAAATAGCATTTATAATGTGCTCATACCTAGAGCCACTTTTCGGTTACGGTGACTTCTC
AAAAAGACAGTGGGACATGCAAATATTACAGTGCGTCCCGCCCCTGGTAGGTCTACGCTAGGACGCACGCGCACT
ACGGTTCCCGCCTATAGACTGCGCTGGCGATTCCTGGGAGCGGACTGATGACGTCAGCGTTCGGGATCC
>H1_47-H1_51
(SEQ ID NO: 971)
TGGCAAACAACGCCGGCGCAAACAGCATTTATAATGTGCTCATACCTAGAGCCACTTTTCGGTTACGGTGACTTC
TCAACAAGACATTGGGACATGCAAATATTACAGTGCGTCCCGCCCCTGGTGTAGTTCTACGCTAGGACGCACGCG
CACTACGGTTCCCGCCTATAGACGACTGCGCTGGCGATTCCTGGGAGCGGACTGATGACGTCAGCGTTCGGGATC
C
>H1_47-H1_94
(SEQ ID NO: 972)
TGGCAAACAACGCCGGCGCAAACAGCATTTATAATGTGCTCATACCTAGAGCCACTTTTCGGTTACGGTGACTTC
TCAAAAAGACATTGGGACATGCAAATATTACAGTGCGTCCCGCCCCTGGTGTAGTTCTACGCTAGGACGCACGCG
CACTACGGTTCCCGCCTATAGACGACTGCGCTGGCGATTCCTGGGAGCGGACTGATGACGTCAGCGTTCGGGATC
C
>H1_53-H1_57
(SEQ ID NO: 973)
TGCCAAACAACGCGCGCAAACAGCATTTATAATGCACTCATAAGTAGAGCCACTTTTCGGTTATGGTGACTTCTC
ACAAGGAATTGGGACATGCAAATATTACAGTGCGTCCCGCCCCTGGTAGTTCTGCGCTAGGACGCAGACGCACTA
CGGTTCCCGCCTTTAGACCGCGCTGGCGATTCCTGGGAGCGGACTGATGACGTCAGCGTTCGGGATCC
>H1_59-H1_54
(SEQ ID NO: 974)
TGCCAAACAACGCGCGCAAACAGCATTTATAATGCACTCATAAGTAGAGCCACTTTTCGGTTATGGTGACTTCTC
ACAAGGAATTGGGACATGCAAATATTACAGTGCGTCCCGCCCCTGGTAGTTCTGCGCTAGGACGCAGACGCACTA
CGGTTCCCGCCTTTAGACTGCGCTGGCGATTCCTGGGAGCGGACTGATGACGTCAGCGTTCGGGATCC
>H1_59-H1_60
(SEQ ID NO: 975)
TGCCAAACAACGCGCGCAAACAGCATTTATAATGCACTCATAAGTAGAGCCACTTTTCGGTTATGGTGACTTCTC
ACAAGGAATTGGGACATGCAAATATTACAGTGCGTCCCGCCCCTGGTAGTTCTACGGACGCAGACGCACTACGGT
TCCCGCCTTTAGACTGCGCTGGCGATTCCTGGGAGCGGACTGATGACGTCAGCGTTCGGGATCC
>H1_61-H1_62
(SEQ ID NO: 976)
TGGCAAACACCGCGCGCAACCAGCATTTATAATGCGCTCGTACCTAAAGGCACTTGTCGGTTACGGTGACTTCCC
ACAAGACATTGCGACATGCAAATACTACAGTGCGTCCCGCCCCTGGTAGTTCCACGCTGGGACGCACACGCAGTA
CGGTTCCCGCCTTTAGACTGCGCTGGCGATTCCTGGGAGAGGATTGATGACGTCAGCGTTCGGGCTCC
>H1_63-H1_64
(SEQ ID NO: 977)
CGGCACAAAACGCGGGCAAACAGCATTTATAATGCGCTCATACCTAAAGCCACTTTACGGTTACGGTGACTTCCC
ACAAGACATTGCGACATGCAAATATTTTAGTGCGTCCCGCCCCTGGTAGTTCCACGCTAGGACACACACGCACTA
TGCTTCCGGCCTTTAGACTGCGCCGGTGATTCCAGGGAGCGGACTGATGACGTCAGCGTTCCGGCTCC
>H1_65-H1_63
(SEQ ID NO: 978)
CGGCAAAAAACGCGGGCAAACAGCATTTATAATGCGCTCATACCTAAAGCCACTTTACGGTTACGGTGACTTCCC
ACAAGACATTGCGACATGCAAATATTTTAGTGCGTCCCGCCCCTGGTAGTTCCACGCTAGGACACACACGCACTA
TGGTTCCGGCCTTTAGACTGCGCCGGTGATTCCAGGGAGCGGACTGATGACGTCAGCGTTCGGGCTCC
>H1_66-H1_65
(SEQ ID NO: 979)
CGGCAAACAACGCGCGCAAACAGCATTTATAATGCGCTCATACCTAAAGCCACTTTACGGTTACGGTGACTTCCC
ACAAGACATTGCGACATGCAAATATTTTAGTGCGTCCCGCCCCTGGTAGTTCCACGCTAGGACGCACACGCACTA
CGGTTCCCGCCTTTAGACTGCGCTGGCGATTCCAGGGAGCGGACTGATGACGTCAGCGTTCGGGCTCC
>H1_67-H1_69
(SEQ ID NO: 980)
TGGCGAATAACACGCGCAAAGAGCATTTATAACGCGCTCAGACCTAAAGCCACTTGTCGCTTACGGTGACTTCCC
ATAAGACATTGCAATATGCAAATACTCCAGTGCGTCCCGCCCCTGGCAGTTCCACGCTGGGACGTGCACGCACTA
CGGTTCCCGCCTTTAGACTGCGCTCGCGATTCCTGGGAGAGGGCTGATGACGTCAGCGTTCGGGCTCC
>H1_70-H1_71
(SEQ ID NO: 981)
TGGCGAAAATCACGCGCAAAGAGCATTTATAACGTGCTCAGACCTAAAGCCACTTGTCGCTTACGGTGACTCCCC
ATAAGACATTGCGATATGCAAATACTGCAGTGCGTCCCGCCCCTGGCAGTTCCACGCTGGGACGTACACGTACTA
CGGTTCCCGCCTTTAGACTGCGCTCGCGATTCCTGGGAGAGGGCTGATGACGTCAGCGTTCGGGCTCC
>H1_70-H1_76
(SEQ ID NO: 982)
TGGCGAAAAACACGCGCAAAGAGCATTTATAACGTGCTCAGACCTAAAGCCACTTGTCGCTTACGGTGACTCCCC
ATAAGACATTGCGATATGCAAATACTGCAGTGCGTCCCGCCCCTGGCAGTTCCACGCTGGGACGTGCACGCACTA
CGGTTCCCGCCTTTAGACTGCGCTCGCGATTCCTGGGAGAGGGCTGATGACGTCAGCGTTCGGGCTCC
>H1_77-H1_79
(SEQ ID NO: 983)
CGGCGAAAAACACGCGCAAAGAGCGTTTATAATGCGCTCAGACCTAAAGTAACTTGTCACTTACGGTGACTTCCC
ATAAGACATTGCGATATGCAAATATTCCAGTGCGTCCCGCCCCTGGCAGTTCCACGCCGGGACGTGCACGCACTA
CGGTTCCCGCCTTTAGACTGCGCTGGCGATTCCTGGGAGAGGGCTGATGACGTCAGCGTGCGGGCTCC
>H1_77-H1_80
(SEQ ID NO: 984)
CGGCGAAAAACACGCGCAAAGAGCGTTTATAACGCGCTCAGACCTAAAGCTACTTGTCACTTACGGTGACTTCCC
ATAAGACATTGCGATATGCAAATATTCCAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTGCGGGCTCC
>H1_77-H1_81
(SEQ ID NO: 985)
CGGCGAAAAACACGCGCAAAGAGCGTTTATAACGCGCTCAGACCTAAAGCTACTTGTCACTTACGGTGACTTCCC
ATAAGACATTGCGATATGCAAATATTCCAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTTCGGGCTCC
>H1_77-H1_82
(SEQ ID NO: 986)
TGGCGAAAAACACGCGCAAAGAGCATTTATAACGCGCTCAGACCTAAAGCCACTTGTCGCTTACGGTGACTTCCC
ATAAGACATTGCGATATGCAAATATTACAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTTCGGGCTCC
>H1_82-H1_67
(SEQ ID NO: 987)
TGGCGAAAAACACGCGCAAAGAGCATTTATAACGCGCTCAGACCTAAAGCCACTTGTCGCTTACGGTGACTTCCC
ATAAGACATTGCGATATGCAAATACTACAGTGCGTCCCGCCCCTGGCAGTTCCACGCTGGGACGTGCACGCACTA
CGGTTCCCGCCTTTAGACTGCGCTCGCGATTCCTGGGAGAGGGCTGATGACGTCAGCGTTCGGGCTCC
>H1_83-H1_77
(SEQ ID NO: 988)
TGGCGAAAAACGCGCGCAAAGAGCATTTATAATGCGCTCAGACCTAAAGCCACTTGTCGCTTACGGTGACTTCCC
ATAAGACATTGCGATATGCAAATATTACAGTGCGTCCCGCCCCTGGCAGTTCCACGCTGGGACGTGCACGCACTA
CGGTTCCCGCCTTTAGACTGCGCTGGCGATTCCTGGGAGAGGGCTGATGACGTCAGCGTTCGGGCTCC
>H1_83-H1_87
(SEQ ID NO: 989)
TGGAGGAGAACGCGCGCAAAGAGCATTTATAATGCGCGCAGACCTAAAGCCACTTGTCGCTTACGGTGACTTCCC
ATAAGACATTGCGATATGCAAATATTACAGTGCGTCCCGCCCCTGGCAGTTCCACGCTGGGACGTGCACGCGCTA
CGGTTCCCGCCTTTAGACTGCGCTGGCGATTCCTGGGAGAGGGCTGATGACGTCAGCATTCGGGCTCC
>H1_95-H1_140
(SEQ ID NO: 990)
TGGCAAAAACTGAGCTCAAGCAGCATTTATAAGGCTCCCAAACCTAAAGACATTTGTCGGTTATGGTGACTTCCC
ACAACACATTGCGACATGCAAATACTGCGGAGCGTACCCCCTCCCCTGGCAACTCCTCGCTGGGACGCACGCGCG
CTACGTGTTCCCGCCTTTTGACTGCGCCGGCGATACCTGGGAGAGGGTTGATGACGTCAGCGTTCGGGCTCC
>H1_98-H1_100
(SEQ ID NO: 991)
TGGGAAAGGGTGGGCTCACGCAGCCTTTATAAGGCTCCCAAACTTAAAGACATTTCTCGGTTATGGCGACTTCCC
ACAAGACATAGCGACATGCAAATACTGCAGACCTGTGGCGCCGACCCGGTCCTGTGCAGCCATCTTTACGGCTGG
GACGCACGCGCGCTGCGTGTTCCCGCCCTGTGACTGCGCCGGCGATTACTGGGAGAGGATTGATGACGTCAACGT
TCGGGTTCC
>H1_100-H1_101
(SEQ ID NO: 992)
TGAGAGAGGGTGGGCTCACGCCACCTTTATAAGGCTCCCAAACTTAAAGACATTTCTCGGTTATGGCGACTTCCC
ACAACACATAGCGACATGCAAATACTGCAGACCTGTGGCGCCGACCCGGTCCTGTGCAGCCATCTTTACGGCTGG
GACGCACGCGCGCTGCGTGTTCCCGCCCTGTGACTGCGCCGGCGATTACTGGGAGAGGATTGATGACGTCAACGT
TCGGGTTCC
>H1_109-H1_107
(SEQ ID NO: 993)
CGTAGGAAAACTGCTTCTGTGAGCACTTATAAAACTCCCATAAGTAGAGAGATTTCATAGTTATGGTGACTTCCC
ATAAGACATTGCGACATGCAAATATTGTGGCGCGTTCGTCCCCGTCCGGTGCAGGCAGCTTCGCTCCAGGACGCA
CGCGCAATACATGTTCCCGCCTTGAGACTGCGCCGGCAGATTCCTAGGAAGTGGTTGATGACGTCGATGTTAGGG
ATCC
>H1_111-H1_109
(SEQ ID NO: 994)
CGTAGGAAAACTGCTTCTGTGAGCACTTATAAAACTCCCATAAGTAGAGAGATTTCATAGTTATGGTGACTTCCC
ATAAGACATTGCGACATGCAAATATTGTGGCGCGTTCGTCCCCGTCCGGTGCAGGCAGCTTCGCTCCAGGACGCA
CGCGCAATACATGTTCCCGCCTTGAGACTGCGCCGGCCGATTCCTAGGAAGTGGTTGATGACGTCGATGTTGGGG
CTCC
>H1_112-H1_111
(SEQ ID NO: 995)
CGTAGGAAAACTGCTTCTGTGAGCACTTATAAAACTCCCATAAGTAGAGAGATTTCATAGTTATGGTGACTTCCC
ATAAGACATTGCGACATGCAAATATTGTGGCGCGTTCGTCCCCGTCCGGTGCAGGCAGCTTCGCTCCAGGACGCA
CGCGCACTACATGTTCCCGCCTTGAGACTGCGCCGGCCGATTCCTAGGAAGTGGTTGATGACGTCGATGTTGGGG
CTCC
>H1_113-H1_112
(SEQ ID NO: 996)
CGGAGAAAACCTGCTTCACCGAGCATTTATAAAGCTCCCATACTTAAAGAGATTTCATAGTTATGGTGACTTCCC
ACAAGACATTGCGACATGCAAATATTGTGGAGCGTACTTCCCCGTCCTGTGCAGGCAGCTTCCCGCCAGGACGCA
CGCGCGCTGCGTGTTCCCGCCTTGAGACTGCGCCGGCGATTTCCTAGGAGGGTGGTTGATGACGTCAATGTTCGG
GCTCC
>H1_114-H1_121
(SEQ ID NO: 997)
TGCCGAAAGTTTAGCTCAACCTGCATTTATAAGGCTCCCAAACCTAAAGACATTTGTCGGTTATGGTGACTTCCC
GCAACACATTGCGACATGCAAATACTGCGGAGCGTACCTCCCCTGGCAACTCCTCGCTGGGACGCACGCGCGCTG
CGTGCTCCCGCCTTTTGACTGCGCCGGCGATACTTGGGAGAGGGTTGATGACGTCAGCGTTCTGGCTCC
>H1_117-H1_115
(SEQ ID NO: 998)
TGCCGAAAGTTTAGCTCAACCTGCATTTATAAAGCTCCCAAACCTAAAGACATTTGTCGGTTATGGTGACTTCCC
GCAACACATTGCGACATGCAAATACTGCGGAGTGCACCTCCCCTGGCAACTCCTCGCTGGGACGCACGCGCGCTG
CGTGCTCCCGCCTTTTGACTGCGCCGGCGATACTTGGGAGAGGGTTGATGACGTCAGCGTTCTGGCTCC
>H1_118-H1_114
(SEQ ID NO: 999)
TGCCGAAAGTTTAGCTCAACCTGCATTTATAAGGCTCCCAAACCTAAAGACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTGCGTGCTCCCGCCTTTTGACTGCGCCGGCGATACTTGGGAGAGGGTTGATGACGTCAGCGTTCTGGCTCC
>H1_118-H1_122
(SEQ ID NO: 1000)
TGCCGAAAGTTTAGCTCAACCTGCATTTATAAGGCTCCCAAACCTAAAGACATTTGTCGGTTATGGTGACTTCCC
GCAACACATTGCGACATGCAAATACTGCGGAGTGCACCCCCTCCCCTGGCAACTCCTCGCTGGGACGCACGCGCG
CTGCGTGCTCCCGCCTTTTGACTGCGCCGGCGATACTTGGGAGAGGGTTGATGACGTCAGCGTTCTGGCTCC
>H1_118-H1_123
(SEQ ID NO: 1001)
TGCCGAAAATTTAGCTCAAGCCGCATTTATAAGGCTCCCAAACCTAAATACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTACGTGCTCCCGCCTTTTGACTGCGCCGGCGATACTTGGGAGAGGGTTGATGACGTCAGCGTTCTGGCTCC
>H1_124-H1_126
(SEQ ID NO: 1002)
CGCCGAAAACCAGGCTCAAGCCACATTTATAAGGCTCCCAAAGCGAAATACATTTGTCGGTTATGGTGACTTCCC
GCAACACATTGCGACATGCAAATACTGCGGAGCGTTCCTCCCCTGGCAACTCCTCGCTGGGACGCACGCGCACTA
CGTGCTCCCGCCTTTTGACTGCGCCGGCGATACTTGGGAGAGAGTTGATGACGTCAGCGTTCTGGCTCC
>H1_124-H1_129
(SEQ ID NO: 1003)
CGCCGAAAACCAGGCTCAAGCCACATTTATAAGGCTCCCAAACCGAAATACATTTGTCGGTTATGGTGACTTCCC
GCAACACATTGCGACATGCAAATACTGCGGAGCGTTCCTCCCCTGGCAACTCCTCGCTGGGACGCACGCGCACTA
CGTGCTCCCGCCTTTTGACTGCGCCGGCGATACTTGGGAGAGGGTTGATGACGTCAGCGTTCTGGCTCC
>H1_129-H1_127
(SEQ ID NO: 1004)
CGCCGAAAACCAGGCTCAAGCCACATTTATAAGGCTCGCAAACCGAAATACATTTGTCGGTTATGGTGACTTCCC
GCAACACATTGCGACATGCAAATACTGCGGAGCGTTCCTCCCCTGGCAACTCCTCGCTGGGACGCACGCGCACTA
CGTGCTCCCGCCTTTTGACTGCGCCGGCGATACTTGGGAGAGGGTTGATGACGTCAGCGTTCTGGCTCC
>H1_133-H1_132
(SEQ ID NO: 1005)
CGCCGAAAACCAGGCTCAAGCCACATTTATAAGGCTCCCAAACCTAAGTACATTTGTCGGTTATGGTGACTTCCC
GCAACACATTGCGACATGCAAATACTGCGGAGCGTCCCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_134-H1_133
(SEQ ID NO: 1006)
CGCCGAAAACCAGGCTCAAGCCACATTTATAAGGCTCCCAAACCTAAGTACATTTGTCGGTTATGGTGACTTCCC
GCAACACATTGCGACATGCAAATACTGCGGAGCGTTCCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_135-H1_134
(SEQ ID NO: 1007)
CGCCGAAAACCAGGCTCAAGCCACATTTATAAGGCTCCCAAACCTAAATACATTTGTCGGTTATGGTGACTTCCC
GCAACACATTGCGACATGCAAATACTGCGGAGCGTTCCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_136-H1_137
(SEQ ID NO: 1008)
TGCCGAAAACCTAGCTCAAGCCACATTTATAAGGCTCCCAAACCTAAATACATTTGTCGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_137-H1_124
(SEQ ID NO: 1009)
CGCCGAAAACCAGGCTCAAGCCACATTTATAAGGCTCCCAAACCTAAATACATTTGTCGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGCGCCGGCGATACTTGGGAGAGGGTTGATGACGTCAGCGTTCTGGCTCC
>H1_137-H1_138
(SEQ ID NO: 1100)
CGCCGAAAGCCAGGCTCAAGCCACATTTATAAGGCTCCCAAACCTAAATACATTTGTCGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGCGCCGGCGACACTTGGGAGAGGGTTGATGACGTCAGCGTTCTGGCTCC
>H1_140-H1_141
(SEQ ID NO: 1101)
TGGCAAAAACTGAGCTCAAGCCGCATTTATAAGGCTCCCAAACCTAAAGACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTACGTGCTCCCGCCTTTTGACTGCGCCGGCGATACTTGGGAGAGGGTTGATGACGTCAGCGTTCTGGCTCC
>H1_141-H1_118
(SEQ ID NO: 1102)
TGCCGAAAACTTAGCTCAAGCCGCATTTATAAGGCTCCCAAACCTAAATACATTTGTCGGTTATGGTGACTTCCC
GCAACACATTGCGACATGCAAATACTGCGGAGCGTACCCCCTCCCCTGGCAACTCCTCGCTGGGACGCACGCGCG
CTACGTGCTCCCGCCTTTTGACTGCGCCGGCGATACTTGGGAGAGGGTTGATGACGTCAGCGTTCTGGCTCC
>H1_141-H1_139
(SEQ ID NO: 1103)
TGCCGAAAACTTAGCTCACGCCGCACTTATAAGGCTCCCAAACCTAAATACATTTGTAGGTTATGGTGACTTCCC
GCAACACATTGCGACATGCAAATACTGCGGAGCGTACCTCCCCTGGCAACTCCTCGCTGGGACGCACGCGCGCTA
CGTGCTCCCGCCTTTTGACTGAGCCGGCGATACTTGGGAGAGGGTTGATGACGTCAGCGTTCTGGCTCC
>H1_141-H1_142
(SEQ ID NO: 1104)
TGCCGAAAGCTTACCTTCGCCCGCCTTATAAGGCTCCCAAACCTAAATACATTTGTAGGTTATGGTGACTTCCCG
CAACACATTGCGACATGCAAATACTGCGGAGCGTACCTCCCCTGGAAACTCCTCGCTGGGACGCACGCGCGTTAC
GTGCTCCCGCCTTTTGACTGAGCCGGCGATACTTGGGAGAGGGTTGATGACGTCAGCGTTCTGGCTCC
>H1_150-H1_146
(SEQ ID NO: 1105)
TGGGAAAGGGTGGCCCCGCCGAGCATTTATAAGACTCCCATACCTAAAGACATTTCTCAGTTATGGTGATTTCCC
TACAACACACAGCAACATGCAAATATCGAGGGGTGTACCGCCCCTGTCCTTTGTAGACGTCTTCTCTCCAGGACG
CACGCGCGCTGTATTCCCGCCTTGTGACTCTAGGCGGGCGATTCCTGGGAGAGGGTTGATGACGTCCAAGTTCTG
GCTTC
>H1_151-H1_150
(SEQ ID NO: 1106)
TGGGAAAGGGTGGCCCCGCCGAGCATTTATAAGACTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCTCCAGGACG
CACGCGCGCTGTATTCCCGCCTTGTGACTCTAGGCGGGCGATTCCTGGGAGAGGGTTGATGACGTCCAAGTTCTG
GCTTC
>H1_151-H1_153
(SEQ ID NO: 1107)
TGGGAAAGGGTGGCTCCGCCGAGCATTTATAAGACTCCCATACCTAAAGACATTTCTCAGTTATGGTGACTTCCC
ACAACGCACAGCAACATGCAAATATCGAGGGGTGTACCGCCCCTGTCCTTTGTAGACGTCTTCTCTCCAGGACGC
ACGCGCGCTGTATTCCCGCCTTGTGACTAGGCGGGCGATTCCTGGGAGAGGGTTGATGACGCCCAAGTTCTGGCT
TC
>H1_151-H1_155
(SEQ ID NO: 1108)
TGGGAAAGGGTGGCCCCGCCGAGCATTTATAAGACTCCCATACCTAAAGACATTTCTCAGTTATGGTGACTTCCC
ACAACACACAGCAACATGCAAATATCGAGGGGTGTACCGCCCCTGTCCTTTGTAGACGTCTTCTCTCCAGGACGC
ACGCGCGCTGTATTCCCGCCTTGTGACTAGGCGGGCGATTCCTGGGAGAGGGTTGATGACGTCCAAGTTCTGGCT
TC
>H1_157-H1_156
(SEQ ID NO: 1109)
TGGGAAAGGGGGGCTCCGCTGAGCGTTTATAAGGCTCCCATACCTAAAGACATTTCACAGTTATGGTGACTTCCC
ACAACACACAGCAACATGCAAATACAGAGGGGTGTACCGCCCCTGTCCTTTGTAGACGTCTTCTCGCCAGGACGC
ACGCGCGCTGTGTTCCCGCCTTGTGACTCTAGGCGGGCGATTCCTGGGAGAGGGTTGATGACGTCCAAGTTCTGA
CTCC
>H1_157-H1_158
(SEQ ID NO: 1110)
TGGGAGAGGGAGGTTCCGCTGAGCGTTTATAAGGCTCCCATATCTAAAGACATTTCACAGTTATGGTGACTTCCC
ACAACACACAGCAACATGCAAATACAGAGAAGCGTACCACCCCTGTCCTTTGCAGACGTCTTCTAGCCAGGACGC
ACGCGCACTGTGTTCCCGCCTTGTGACTCGAGGCGGGCGATACCTGGGAGAGGGTTGATGACGTCCAAGTTCTGA
CTCC
>H1_157-H1_160
(SEQ ID NO: 1111)
TGGGAAAGGGTGGCTCCGCCGAGCATTTATAAGGCTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCGCCAGGACG
CACGCGCGCTGTGTTCCCGCCTTGTGACTCTAGGCGGGCGATTCCTGGGAGAGGGTTGATGACGTCCAAGTTCTG
ACTCC
>H1_160-H1_151
(SEQ ID NO: 1112)
TGGGAAAGGGTGGCTCCGCCGAGCATTTATAAGGCTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCTCCAGGACG
CACGCGCGCTGTGTTCCCGCCTTGTGACTCTAGGCGGGCGATTCCTGGGAGAGGGTTGATGACGTCCAAGTTCTG
ACTCC
>H1_160-H1_159
(SEQ ID NO: 1113)
CAGGCAAAAGCAGTTCGGCCGAGAATTTATAAGGCTCCAATACCTAAAGACATTTCTCAGTTACGGTGACTTCCC
ACAACACACAGCAACATGCAAATATCGAGAGGTGTACCGCCCCTGTCCTTTGTAGACGTCTTCTCTTCGGGACGC
ACGCGCGCTGTGTTCCCGCCTTATGACTCTAGGCGGGCGATTCCTGGGAGAGGGTTGATGACGTCCAAGTTCTGA
CTCC
>H1_160-H1_161
(SEQ ID NO: 1114)
CAGGCAAAAGCAATTCGGCCGAGAATTTATAAGGCTCCAATACCTAAAGACATTTCTCAGTTACGGTGACTTCCC
ACAACACACAGCAACATGCAAATATCGAGAGGTGTACCGCCCCTGTCCTTTGTAGACGTCTTCTCTTCGGGACGC
ACGCGCGCTGTGTTCCCGCCTTATGACTCTAGGCGGGCGATTCCTGGGAGAGGGTTGATGACGTCCAAGTTCTGA
CTCC
>H1_162-H1_157
(SEQ ID NO: 1115)
TGGGAAAAGGTGGCTCCACAGAGCATTTATAAGGCTCCCATACCTAAAGACATTTCTCAGTTATGGTGACTTCCC
TACAACACACAGCAACATGCAAATATCGAGGGGTGTACCGCCCCTGTCCTTTGTAGACGTCTTCTCGCCAGGACG
CACGCGCGCTGTGTTCCCGCCTTGTGACTCTAGGCGGGCGATTCCTGGGAGAGGGTTGATGACGTCCAAGTTCTG
ACTCC
>H1_163-H1_196
(SEQ ID NO: 1116)
TGGGAAAGGGTGGCCCCACAGAGCATTTATAAGGCTCCCATACCTAAAGACATTTCTCAGTTATGGTGACTTCCC
ACAACGCATAGCAACATGCAAATATCGCGGGGAGTACCTCCCCTGTCCCTTGTACGCGTCTTCTCAGGACGCACG
CACGCGCGCTGTGTTCCCGCCCTGTGACTCCAGGCGGGCAATTCCCGGGAGAGGGTTGCTGACGGGAACGTTCAG
GCTCC
>H1_164-H1_167
(SEQ ID NO: 1117)
TGGGAAAGGGTGGTCCTGAGGCGGATTTATAAGGCTCCCACATCTAAAGGCATTTCACAGTCATGGTGACTTCCC
ACAATACATAGCAACATGCAAATTTCGCGGGGAGTACCTCCCCTGTCCCTTGTACGCGGCTTCTCAGGACGCACG
CACGCGCTCTGTGTTCCCGCCCTGTGACTCTAGGAGGGCAATTCCTGGGACAGTGTTCTGACGGGAACGTTCAGG
CTCC
>H1_166-H1_164
(SEQ ID NO: 1118)
TGGGAAAGGGTGGTCCTGAGGCGGATTTATAAGGCTCCCATATCTAAAGGCATTTCACAGTCATGGTGACTTCCC
ACAATACATAGCAACATGCAAATTTCGCGGGGAGTACCTCCCCTGTCCCTTGTACGCGGCTTCTCAGGACGCACG
CACGCGCTCTGTGTTCCCGCCCTGTGACTCTAGGAGGGCAATTCCTGGGACAGTGTTCTGACGGGAACGTTCAGG
CTCC
>H1_169-H1_165
(SEQ ID NO: 1119)
TGGGAAAAGGTGGTCCTGGGGCGGATTTATAAGGCTCCCATATCTAAAGGCATTTCACAGTCATGGTGACTTCCC
ACAATACATAGCAACATGCAAATATCGCGGGGAGTACCTCCCCTGTCCCTTGTACGCGGCTTCTCAGGACGCACG
CACGCGCTCTGTGTTCCCGCCCTGTGACTCTAGGCGGGCAATTCCTGGGACAGTGTTCTGACGGGAACGTTCAGG
CTCC
>H1_171-H1_172
(SEQ ID NO: 1120)
TGGAAAAGAGTGGCCCCAGAGAGCATTTATAAGGCTCCCGCACCTAAATGCATTTATCAGTTATGGTGACTTCCC
ACAATACATAGCAACATGCAAATATAGCGGGGAGTACCTCCCCTGTCCCTTGTCCGTGTCTTCTCAGGACGCACG
CACGCGCGCTGTGTTCCCGCCCTGTGACTCTAAGCGGGCAATTCCTGGAAGAGGCTTGCTGACGGGAACGTTCAG
GCTCC
>H1_171-H1_173
(SEQ ID NO: 1121)
TGGGAAAGAGTGGCCCCAGAGAGCATTTATAAGGCTCCCGCACCTAAATGCATTTATCAGTTATGGTGACTTCCC
ACAATACATAGCAACATGCAAATATAGCGGGGAGTACCTCCCCTGTCCCTTGTACGTGTCTTCTCAGGACGCACG
CACGCGCGCTGTGTTCCCGCCCTGTGACTCTAAGCGGGCAATTCCTGGAAGAGGCTTGCTGACGGGAACGTTCAG
GCTCC
>H1_175-H1_176
(SEQ ID NO: 1122)
TGGGAAAGGGTGGCCCCAGAGAGCATTTATAAGGCTCCCGCACCTAAATGCATTTATCAGTTATGGTGACTTCCC
ACAATACATAGCAACATGTAAATATCGCGGGGAGTACCTCCCCTGTCCCTTGTACGTGTCTTCTCAGGACGCACG
CACGCGCGCTGTGTTCCCGCCCTGTGACTCTAGGCGGGCAATTCCTGGAAGAGGCTTGCTGACGGGAACGTTCAG
GCTCC
>H1_177-H1_171
(SEQ ID NO: 1123)
TGGGAAAGGGTGGCCCCAGAGAGCATTTATAAGGCTCCCGCACCTAAATGCATTTATCAGTTATGGTGACTTCCC
ACAATACATAGCAACATGCAAATATCGCGGGGAGTACCTCCCCTGTCCCTTGTACGTGTCTTCTCAGGACGCACG
CACGCGCGCTGTGTTCCCGCCCTGTGACTCTAGGCGGGCAATTCCTGGAAGAGGCTTGCTGACGGGAACGTTCAG
GCTCC
>H1_177-H1_178
(SEQ ID NO: 1124)
TGGGAAACGGTGGCCCCAAAGAGCACTTATAAAGCCCCCTCACCTAAATGCATTTATCAGTTATGGTGACTTCCC
ACAATACATAGCAACATGCAAATATCGCGGGGAGTACCTCCCCTGTCCCTTGTACGTGTCTTCTCAGGACGCACG
CACGCGCGCTGTGTTCCCGCCCTGTGACTCTAGGTGGACAATTCCTGGGGGAGGCTTGCTGACGGGAACGTTCCG
GCTCC
>H1_177-H1_406
(SEQ ID NO: 1125)
TGGGAAACGGTGGCCCCAAAGAGCATTTATAAAGCTCCCTCACCTAAATGCATTTATCAGTTATGGTGACTTCCC
ACAATACATAGCAACATGCAAATATCGCGGGGAGTACCTCCCCTGTCCCTTGTACGTGTCTTCTCAGGACGCACG
CACGCGCGCTGTGTTCCCGCCCTGTGACTCTAGGCGGGCAATTCCTGGAAGAGGCTTGCTGACGGGAACGTTCCG
GCTCC
>H1_181-H1_182
(SEQ ID NO: 1126)
TGGGAAAGGGTGGCCCCAGCGAGCATTTATAAGGCTCCCGCATCTAAAGACATTTTACAGTTATGGTGACTTCCC
ACAACGCGTAGCAACATGCAAATATCGCAGGGAGTACCGCCCCTGTCCCTTGTACGCGTCTTCTCAGCACGCACG
CACGCGCGCTGTGTTCCCGCCCTGTGACTCCAGGCGGGTATTTCCCGGGGGGGGTTTGCTGACAAGAACGTTCAG
GCTCC
>H1_182-H1_183
(SEQ ID NO: 1127)
TGGGAAAGGGTGGGCCCAGCGAGCATTTATAAGGCTCCCGCATCTAAAGACATTTTACAATTATGGTGACTTCCC
ACAACGCGTAGCAACATGCAAATATCGCAGGGAGTACCGCCCCTGTCCCTTGTACGCGTCTTCTCAGCACGCACG
CACGCGCGCTGTGTTCCCGCCCTGTGACTCCAGGCGGGTATTTCCCGGGGGGGGTTTGCTGACAAGAACGTTCAG
GCTCC
>H1_184-H1_185
(SEQ ID NO: 1128)
TGGGAAAGGGTGGCCCCAGAGAGCATTTATAAGGCTCCCGCACCTAAAGGCATTTAACAGTTATGGTGACTTCCC
ACAACGCGTAGCAACATGCAAATATCGCAGGGAGTACCGCCCCTGTCCCTTGTACGCATCTTCTCAGGACGCACG
CACGCGCGCTGTGTTCCCGCCCTGTGACTCCAGGCGGGCATTTCCCGGGGGGGGTTTGCTGACAGGAACGTTCAG
GCTCC
>H1_188-H1_162
(SEQ ID NO: 1129)
TGGGAAAAGGTGGCCCCACAGAGCATTTATAAGGCTCCCATACCTAAAGACATTTCTCAGTTATGGTGACTTCCC
TACAATACATAGCAACATGCAAATATCGCGGGGCGTACCTCCCCTGTCCCTTGTAGGCGTCTTCTCAGCCAGGAC
GCACGCGCGCTGTGTTCCCGCCCTGTGACTCTAGGCGGGCGATTCCTGGGAGAGGGTTGATGACGTCAACGTTCG
GGCTCC
>H1_188-H1_163
(SEQ ID NO: 1130)
TGGGAAAGGGTGGCCCCAGAGAGCATTTATAAGGCTCCCATACCTAAAGGCATTTCTCAGTTATGGTGACTTCCC
ACAACGCATAGCAACATGCAAATATCGCGGGGAGTACCTCCCCTGTCCCTTGTACGCGTCTTCTCAGGACGCACG
CACGCGCGCTGTGTTCCCGCCCTGTGACTCCAGGCGGGCAATTCCCGGGAGAGGGTTGCTGACGGGAACGTTCAG
GCTCC
>H1_188-H1_170
(SEQ ID NO: 1131)
TGGGAAAGGGTGGCCCCAGAGAGCATTTATAAGGCTCCCGCACCTAAAGGCATTTTACAGTTATGGTGACTTCCC
ACAACGCGTAGCAACATGCAAATATCGCGGGGAGTACCGCCCCTGTCCCTTGTACGCGTCTTCTCAGGACGCACG
CACGCGCGCTGTGTTCCCGCCCTGTGACTCCAGGCGGGCAATTCCCGGGGGGGGTTTGCTGACGGGAACGTTCAG
GCTCC
>H1_188-H1_177
(SEQ ID NO: 1132)
TGGGAAAGGGTGGCCCCAGAGAGCATTTATAAGGCTCCCATACCTAAAGGCATTTCTCAGTTATGGTGACTTCCC
ACAATACATAGCAACATGCAAATATCGCGGGGAGTACCTCCCCTGTCCCTTGTACGCGTCTTCTCAGGACGCACG
CACGCGCGCTGTGTTCCCGCCCTGTGACTCTAGGCGGGCAATTCCTGGGAGAGGGTTGCTGACGGGAACGTTCAG
GCTCC
>H1_188-H1_179
(SEQ ID NO: 1133)
TGGGAAAGGGTGGCCCCAGCGAGCATTTATAAGGCTCCCGCATCTAAAGACATTTTACAGTTATGGTGACTTCCC
ACAACGCGTAGCAACATGCAAATATCGCAGGGAGTACCGCCCCTGTCCCTTGTACGCGTCTTCTCAGGACGCACG
CACGCGCGCTGTGTTCCCGCCCTGTGACTCCAGGCGGGTATTTCCCGGGGGGGGTTTGCTGACAGGAACGTTCAG
GCTCC
>H1_188-H1_180
(SEQ ID NO: 1134)
TGGGAAAGGGTGGCCCCAGCGAGCATTTATAAGGCTCCCGCATCTAAAGACATTTTACAGTTATGGTGACTTCCC
ACAACGCGTAGCAACATGCAAATATCGCAGGGAGTACCGCCCCTGTCCCTTGTACGCGTCTTCTCAGCACGCACG
CACGCGCGCTGTGTTCCCGCCCTGTGACTCCAGGCGGGTATTTCCCGGGGGGGGTTTGCTGACAGGAACGTTCAG
GCTCC
>H1_188-H1_186
(SEQ ID NO: 1135)
TGGGAAAGGGTGGCCCCACCGAGCATTTATAAGGCTCCCGCATCTAAAGACATTTTACAGTTATGGTGACTTCCC
ACAACGCGTAGCAACATGCAAATATCGCGGAGAGTACCGCCCCTGTCCCATGCACGCGTCTTCTCAGCACGCACG
CACGCGCGCTGTGTTCCCGCCCTGTGACTCCAGGCGGGTATTTCCAGGGGCGGGTTTGCTGACAGGAACGTTCAG
GCTTC
>H1_188-H1_198
(SEQ ID NO: 1136)
TGGGAAAAGGTGGCCCCAGAGAGCATTTATAAGGCTCCCATACCTAAAGGCATTTCTCAGTTATGGTGACTTCCC
ACAATACATAGCAACATGCAAATATCGCGGGGAGTACCTCCCCTGTCCCTTGTACGCGTCTTCTCAGGACGCACG
CACGCGCGCTGTGTTCCCGCCCTGTGACTCTAGGCGGGCAATTCCTGGGAGAGGGTTGCTGACGGGAACGTTCAG
GCTCC
>H1_188-H1_203
(SEQ ID NO: 1137)
TGGGAAAAAGTGGGGCCTCACGCAGCATTTATAAGGCTCCCATACCTAAAGACATTTCACGGTTATGGTGACTTC
CCCCACAATACATAGCGACATGCAAATATCATGGTCCTTCCGCGGGGCGTGCCTCCCCCTGTCCCTTGGCCCGTA
GGCGTCTTCTCAGCCAGGAGACGCACGCGGCGCGCTGCGTGTTCCCGCCCTGTGACTTCTAGGCGGGCGATTCCC
TGGGAGAGGGTTGGATGACGTCAGCATCGCCAACGTTCGGGCTCC
>H1_189-H1_1
(SEQ ID NO: 1138)
TGGGAAAAGGTGGGCCCACGCAGAATTTATAAGGCTCCCATACCTAAAGACATTTTACGATTATGGTGACTTCCC
ACAATACATAGCGACATGCAAATATCGCGGGGCGTGCCTCCCCTGTCCCGTCACAGGCGTCTTCTCAGCCAGGGC
GCACGCGCGCTGCGTGTTCCCGCCCTGTGACTCTGGGCCCGCGATTCCTGGGAGCGGGTTGATGACGTCAGCGTT
CGGGCTCC
>H1_189-H1_192
(SEQ ID NO: 1139)
TGGGAAAGGGTGGACCCACCGAGCATTTATAAGGCTCCCGCATCTAAAGACATTTTACAGTTATGGTGACTTCCC
ACAACGCGTAGCAACATGCAAATATCGTGGAGAGTACCGCCCCTGTCCCATGCACGCGTCTTCTCAGCACGCACG
CACGCGCGCTGTGTTTCCCGCCCTGTGACTCCAGGCGGGTATTTCCAGGGGCGGGTTTGCTGACAGGAACGTTCA
GGCTTC
>H1_189-H1_227
(SEQ ID NO: 1140)
TGGGAAAAGGTGGGCCCACGCAGAATTTATAAGGCTCCCATACCTAAAGACATTTCACGATTATGGTGACTTCCC
ACAATACATAGCGACATGCAAATATCGCGGGGCGTGCCTCCCCCTGTCCCGTACCCCACAGGCGTCTTCTCAGCC
AGGGCGCACGCGCGCTGCGTGTTCCCGCCCTGAGTGACTAGGGATTCTGGGCCCGCGATTCCCGTGGGAGCGGGT
TGATGACGTCAGCGTTCGGGCTCC
>H1_189-H1_234
(SEQ ID NO: 1141)
TGGGAAAAGGTGGGCCCACGCAGAATTTATAAGGCTCCCATACCTAAAGACATTTCACGATTATGGTGACTTCCC
ACAATACATAGCGACATGCAAATATCGCGGGGCGTGCCTCCCCTGTCCCGTACCCCACAGGCGTCTTCTCAGCCA
GGGCGCACGCGCGCTGCGTGTTCCCGCCCTGAGTGACTAGGGATTCTGGGCCCGCGATTCCCGTGGGAGCGGGTT
GATGACGTCAGCGTTCGGGCTCC
>H1_189-H1_237
(SEQ ID NO: 1142)
TGGGAAAAGGTGGGCCCACGCAGAATTTATAAGGCTCCCATACCTAAAGACATTTTACGATTATGGTGACTTCCC
ACAATACATAGCGACATGCAAATATCGCGGGGCGTGCCTCCCCTGTCCCGTCACAGGCGTCTTCTCAGCCAGGGC
GCACGCGCGCTGCGTGTTCCCGCCCTGAGTGACTCTGGGCCCGCGATTCCCGTGGGAGCGGGTTGATGACGTCAG
CGTTCGGGCTCC
>H1_189-H1_286
(SEQ ID NO: 1143)
TGGGAAAAGGTGGGCCCACGGAGAATTTATAAGGCTCCCATACCTAAAGACATTTTACGATTATGGTGACTTCCC
ACAACACATAGCGACATGCAAATATCGCGGGGCGTGCCTCCCCTGTCCCGTACAGGCGTCTTCTCAGCCAGGGCG
CACGCGCGCTGCGTGTTCCCGCCCTGTGACTCCGGGCCCGCGATTCCTGGGAGCGGGTTGATGACGTCAGCGTTC
GGGCTCC
>H1_195-H1_184
(SEQ ID NO: 1144)
TGGGAAAGGGTGGCCCCAGAGAGCATTTATAAGGCTCCCGCACCTAAAGGCATTTTACAGTTATGGTGACTTCCC
ACAACGCGTAGCAACATGCAAATATCGCAGGGAGTACCGCCCCTGTCCCTTGTACGCGTCTTCTCAGGACGCACG
CACGCGCGCTGTGTTCCCGCCCTGTGACTCCAGGCGGGCATTTCCCGGGGCGGGTTTGCTGACAGGAACGTTCAG
GCTCC
>H1_196-H1_197
(SEQ ID NO: 1145)
TGAGAAAGGGTGGCTCCACAGAGCATTTATAAGGCTCCCATACCTAAAGACATTTCTCAGTTATGGTGACTTCCC
ACAACGCATAGCAACATGCAAATATCGCGGGGAGTACCTCCCCTGTCCCTTGTACGCGTCTTCTCAGGACGCACG
CACGCGCGCTGTGTTCCCGCCCTGTGACTCCAGGCGGGCAATTCTCGGGAGGGGGTTGCTGACGGGAACGTTCAG
GCTCC
>H1_199-H1_200
(SEQ ID NO: 1146)
TGGGGAAAAACAGCTCACGGCGGCATTTATAAGACTCACAGATCTAAAGCCATTTCACGAATAGGGTGACTTCCC
ACAATACACAGCGACATGCAAACATAGCGGGGCGTGCCTTTCCTGTACCCTGTGGGCATCTCTCCTGGACGCACG
CGCGCCGGGTGTTCCCGCGCTGTGACTCTAGGCAAGCGCTTCCTGGGAGAGAGTTGATGACGGCAGCATTCGGGC
TCC
>H1_203-H1_199
(SEQ ID NO: 1147)
TGGGGAAAAGCGGGCTCCAGGCAGCATTTATAAGACTCACATATCTAAAGACATTTCACGGTTAGGGTGACTTCC
CACAATACACAGCGACATGCAAATATCGCGGGGCGTGCCTCCCCTGTCCCTTGTGGGCATCTTCTCGCCTGGACG
CACGCGCGCCGCGTGTTCCCGCCCTGTGACTCTAGGCGGGCGATTCCTGGGAGAGGGTTGATGACGTCAACATTC
GGGCTCC
>H1_203-H1_202
(SEQ ID NO: 1148)
CGGAGCAAACAGGCCACCAGGCAGCCTTTATAAGACTCACATATCTAAAGACATTTCACAGTTAGGGTGACTTCC
CACAGTACACAGCGATATGCAAATATCGCGGAGCGTGCCTCCCCAGTCTCTGGCGGGCATCTTCTCGCCTACACG
CACGCGCGCCGCGTGTTCCCGCCCTGTGACGCTAGGCGGGCCATTCATGGGAGAGGGTTGATGACGTCAACATTC
GGACTCC
>H1_203-H1_206
(SEQ ID NO: 1149)
TGGAGAAAAGCGGGCTCCAGGCAGCATTTATAAGACTCACATATCTAAAGACATTTCACAGTTAGGGTGACTTCC
CACAATACACAGCGACATGCAAATATCGCGGAGCGTGCCTCCCCTGTCTCTTGTGGGCATCTTCTCGCCTGGACG
CACGCGCGCCGCGTGTTCCCGCCCTGTGACGCTAGGCGGGCGATTCCTGGGAGAGGGTTGATGACGTCAACATTC
GGGCTCC
>H1_203-H1_304
(SEQ ID NO: 1150)
TGGGAAAAAGAGGGGCTTCACGCAGCATTTATAAGGCTCCCATATCTAAAGACATTTCACGGTTAGGGTGACTTC
CCCCACAATACATAGCGACATGCAAATATCATGGTCCTTCCGCGGGGCGTGCCTCCCCCTGTCCCTTGGCCCGTG
GGCATCTTCTCGCCAGGAGACGCACGCGGCGCGCTGCGTGTTCCCGCCCTGTGACTTCTAGGCGGGCGATTCCCT
GGGAGAGGGTTGGATGACGTCAGCATCGCCAACATTCGGGCTCC
>H1_206-H1_207
(SEQ ID NO: 1151)
TGAAGAAAGGCGGCTCTAAGCAGCATTTATAAGACTCACATATCTGAAGACATTTCACAGTTAGGGTGACTTCCC
ACAAGACACAGCGACATGCAAATATCGCGGAATGTGCTTCCCCTGTCTCCTGTGGGCATCTTCTCGCCTGGACGC
ACGCGCACCGCGTGTTCCCGCCCTGTGACGCTAGGCGGGCGATTCCTGGGAGAGGGTTGATGACGTCAACACTCG
GGCTCC
>H1_210-H1_208
(SEQ ID NO: 1152)
TGGGAAAGGGTGGTCCCATACAGAACTTATAAGATTCCCATATCCAAAGACATTTCACGTTTATGGTGATTTCCC
AGAACACATAGCGACATGCAAATATTGCAGGGCGCCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_210-H1_209
(SEQ ID NO: 1153)
TGGGAAAGGGTGGTCCCACACAGAACTTATAAGACTCCCATATCCAAAGACATTTCACGTTTATGGTGATTTCCC
AGAACACATAGCGACATGCAAATATTGCAGGGCGCCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_210-H1_212
(SEQ ID NO: 1154)
TGGGGAAGGGTGGTCCCATACAGAACTTATAAGATTCCCATACCCAAAGACATTTCACGTTTATGGTGACTTCCC
AGAAGACATAGCGACATGCAAATATTGCAGGGCGTCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_210-H1_220
(SEQ ID NO: 1155)
TGGGGAAGGGTGGTCCCATACAGAACTTATAAGATTCCCATACCCAAAGACATTTCACGTTTATGGTGACTTCCC
AGAAGACATAGCGACATGCAAATATTGCAGGGCGTCACTCCCCCTGTCCCTCAACAGTCATCTTCCTGCCAGGGC
GCACGCGCGCTGGGTGTTCCCGCCTAGTGACACTGGGCCCGCGATTCCTTGGAGCGGGTTGATGACGTCAGCGTT
CGAATTCC
>H1_210-H1_225
(SEQ ID NO: 1156)
TGGGAAAGGGTGGTCCCATACAGAACTTATAAGATTCCCATACCCAAAGACATTTCACGTTTATGGTGACTTCCC
AGAACACATAGCGACATGCAAATATTGCAGGGCGTCACTCCCCTGTCCCTCACAGCCATCTTCCTGCCAGGGCGC
ACGCGCGCTGGGTGTTCCCGCCTAGTGACACTGGGCCCGCGATTCCTTGGAGCGGGTTGATGACGTCAGCGTTCG
AATTCC
>H1_213-H1_219
(SEQ ID NO: 1157)
TGGGGAAAGGTGGTCCCATACAGAACTTATAAGATTCCCATACTCAAAGACATTTCACGTTTATGGTGACTTCCC
AGAAGACACAGCGACATGCAAATATTGTAGGGCGTCACACCCCCTGTCCCTCACAGTCATCTTCCTGCCAGGGCG
CACGCGCGCTGGGTGTTCCCGCGTAGTGACACTGGGCCCGCGATTCCTTGGAGCGGGTTGATGACGTCAGCGTTC
GAATTCC
>H1_219-H1_218
(SEQ ID NO: 1158)
TGGGGAAAGGTGGTCCCACACAGAACTTATAAGATTCCCATACTCAAAGACATTTCTCGTTTATGGTGACTTCCC
AGAAGACACAGCGACATGCAAATATTGTAGGGCGTCACACCCCTGTCCCTCACAGTCATCTTCCTGCCAGGGCGC
ACGCGCGCTGGGTGTTCCCGCGTAGTGACACTGGGCCCGCGATTCCTTGGAGCGGGTTGATGACGTCAGCGTTCG
AATTCC
>H1_220-H1_222
(SEQ ID NO: 1159)
TGGGGAAGGGTGGTCCCATACAGAACTTATAAGATTCCCATACTCAAAGACATTTCACGTTTATGGTGACTTCCC
AGAAGACATAGCGACATGCAAATATTGCAGGGCGTCACACCCCCTGTCCCTCAACAGTCATCTTCCTGCCAGGGC
GCACGCGCGCTGGGTGTTCCCGCGTAGTGACACTGGGCCCGCGATTCCTTGGAGCGGGTTGATGACGTCAGCGTT
CGAATTCC
>H1_220-H1_223
(SEQ ID NO: 1160)
TGGGGAAGGGTGGTCCTACACAGAACTTATAAGACTCCCATACTCAAAGACATTTCACGTTTATGGTGACTTCCC
AGAAGACATAGCGACATGCAAATATTGCAGGGCGTCACACCCCCTGTCCCTTACAGCCATCTTCCTGCCAGGGCG
CACGCGCGCTGGGTGTTCCCGCGTAGTGACACTGGGCCCGCGATTCCTTGGAGCGGGTTGATGACGTCAGCGTTC
GAATTCC
>H1_220-H1_224
(SEQ ID NO: 1161)
TGGGGAAGGGTGGTCCTACACAGAACTTATAAGACTCCCATACTCAAAGACATTTCACGTTTATGGTGACTTCCC
AGAAGACATAGCGACATGCAAATATTGCAGGGCGTCACACCCCCTGTCCCTTAACAGTCATCTTCCTGCCAGGGC
GCACGCGCGCTGGGTGTTCCCGCGTAGTGACACTGGGCCCGCGATTCCTTGGAGCGGGTTGATGACGTCAGCGTT
CGAATTCC
>H1_222-H1_213
(SEQ ID NO: 1162)
TGGGGAAGGGTGGTCCCATACAGAACTTATAAGATTCCCATACTCAAAGACATTTCACGTTTATGGTGACTTCCC
AGAAGACATAGCGACATGCAAATATTGCAGGGCGTCACACCCCCTGTCCCTCACAGTCATCTTCCTGCCAGGGCG
CACGCGCGCTGGGTGTTCCCGCGTAGTGACACTGGGCCCGCGATTCCTTGGAGCGGGTTGATGACGTCAGCGTTC
GAATTCC
>H1_227-H1_210
(SEQ ID NO: 1163)
TGGGGAAGGGTGGTCCCACACAGAACTTATAAGATTCCCATACCCAAAGACATTTCACGATTATGGTGACTTCCC
AGAAGACATAGCGACATGCAAATATTGCAGGGCGTGCCTCCCCCTGTCCCTCAACAGTCGTCTTCCTGCCAGGGC
GCACGCGCGCTGGGTGTTCCCGCCTAGTGACACTGGGCCCGCGATTCCTTGGAGCGGGTTGATGACGTCAGCGTT
CGAATTCC
>H1_227-H1_226
(SEQ ID NO: 1164)
TGGGGAAGGGTGGTCCTACACAGAACTTATAAGATTCCCATACCCAAAGACATTTCACGATTATGGTGACTTCCC
AGAAGACACAGCGACATGCAAATATTGCAGGTCGTGCCTCGCCTGTCCCTCACAGTCGTCTTCCTGCCAGGGCGC
ACGCGCGCTGGGTGTCCCGCCAACTGACACTGGGCTCGCGATTCCTTGGAGCGGGTTGATGACGTCAGCGTTCGA
ATTCC
>H1_227-H1_228
(SEQ ID NO: 1165)
TGGGGAAGGGTGGTCCCACACAGAACTTATAAGATTCCCATACCCAAAGACATTTCACGATTATGGTGACTTCCC
AGAAGACACAGCGACATGCAAATATTGCAGGTCGTGCCTCGCCTGTCCCTCACAGTCGTCTTCCTGCCAGGGCGC
ACGCGCGCTGGGTTTCCCGCCAACTGACACTGGGCTCGCGATTCCTTGGAGCGGGTTGATGACGTCAGCGTTCGA
ATTCC
>H1_227-H1_230
(SEQ ID NO: 1166)
TGGGGAAGGGTGGTCCTACGCAGAACTTATAAGATTCCCATACCCAAAGACATTTCACGATTATGGTGACTTCCC
AGAATACACAGCGACATGCAAATATTGCAGGTCGTGCCTCGCCTGTCCCTCACAGTCGTCTTCCTGCCAGGGCGC
ACGCGCGCTGGGTGTCCCGCCAACTGACACTGGGCTCGCGATTCCTTGGAGCGGGTTGATGACGTCAGCGTTCGA
ATTCC
>H1_231-H1_232
(SEQ ID NO: 1167)
TGAGGAAAAATGGTTCCACACAGAATTTATAAGGTTCCCAAATCTAAAGACATTTCACCATTATGGTGATTTCCC
ACAACACATAGCGACATGCAAATATCTCAGAGCGTACCTCCCCTGTCCTATACGGGCGTCAACTCGCCAGGGCGC
ACGCGCGCTGTGTGTTTCCCGCCTGTGACTCGGGACTCTGGGCCCGCGATTCCTCGGAGCGGGTTGAGAACGTCA
GCTCCGGTGCTTC
>H1_233-H1_231
(SEQ ID NO: 1168)
TGAGGAAAAGTGGTTCCACACAGAATTTATAAGGTTCCCAAATCTAAAGACATTTCACCATTATGGTGATTTCCC
ACAACACATAGCGACATGCAAATATCTCAGAGCGTACCTCCCCTGTCCTATACGGGCGTCAACTCGCCAGGGCGC
ACGCGCGCTGTGTGTTTCCCGCCTGTGACTCGGGACTCTGGGCCCGCGATTCCTCGGAGCGGGTTGATAACGTCA
GCTCCGGTGCTTC
>H1_234-H1_235
(SEQ ID NO: 1169)
TGGGAAAAGGTGGGCCCACACAGAATTTATAAGGCTCCCATACCTAAAGACATTTCACGATTATGGTGACTTCCC
ACAATACATAGCGACATGCAAATATCGCGGGGCGTGCCTCCCCTGTCCCGTACCCCACAGGCGTCTTCTCGCCAG
GGCGCACGCGCGCTGCGTGTTCCCGCCCTGTGACTAGGGATTCTGGGCCCGCGATTCCTGGGAGCGGGTTGATGA
CGTCAGCGTTCGGGCTCC
>H1_235-H1_233
(SEQ ID NO: 1170)
TGAGGAAAAGTGGGCCCACACAGAATTTATAAGGTTCCCAAACCTAAAGACATTTCACCATTATGGTGACTTCCC
ACAATACATAGCGACATGCAAATATCTCAGGGCGTGCCTCCCCTGTCCCGTACCCCACGGGCGTCAACTCGCCAG
GGCGCACGCGCGCTGCGTGTTTCCCGCCTGTGACTCGGGACTCTGGGCCCGCGATTCCTGGGAGCGGGTTGATGA
CGTCAGCTCTGGGGCTTC
>H1_238-H1_239
(SEQ ID NO: 1171)
TGGCAGAAAGCGGCCCGCCGCCGCATTTATAAGGCTCTCCCACCTAAAGCCATATAATGGTTATGGTGACTTCCC
AGAATACATGGCAACATGCAAATATCGTGCGGTATACCTCCCCTGTCGCGCGTAGGCGTCTCCTCCCCTGGACGC
ACGGGCGCCGCATGTTCCCGCCCTATGACTCTGGGCCGGCGACTACGGGAGAGAGCTGATGACGTGACCGCGACC
GCTCGGGCTCC
>H1_241-H1_238
(SEQ ID NO: 1172)
TGGGAAAAAGCGGCCCCCCGCCGCATTTATAAGGCTCTCCCACCTAAAGACATTTAACGGTTATGGTGACTTCCC
ACAATACATAGCAACATGCAAATATCGCGCGGTATACCTCCCCTGTCGCGCGTAGGCGTCTCCTCCCCTGGACGC
ACGGGCGCTGCGTGTTCCCGCCCTGTGACTCTGGGCCGGCGACTACGGGAGAGAGCTGATGACGTGACCGCGACC
GCTCGGGCTCC
>H1_242-H1_243
(SEQ ID NO: 1173)
TGGGAAGTAAGAGATTCACGCCGGTTATATAAGATTCCTGTAACTAAAGAAATTTCAAGGATAGGGTGACTTCCC
ACAATACAAAGCGACATGCAAATATCGCGGGGCGTGCCTGTCCTGACCTTTGTGAGACTCTTCGCTAGGACGCAG
GCGTGCTGCGAGTTCCCGCCTTATCGGCGAGTCCTGGGGGAGAGTTGATGACGCCAACATTCGGGCTCC
>H1_242-H1_248
(SEQ ID NO: 1174)
TGGGAAAAAAAGGCTTCACGCAGATTATATAAGGTTCCTGTACCTAAAGACATTTCAAGGTTAGGGTGACTTCCC
ACAATACATAGCGACATGCAAATATAGCGGGGCGTGCCTCCCCTGTCCCTTGTGGGCGTCTTCTCGCTAGGACGC
ACGCGCGCTGCGTGTTCCCGCCTTGTGACTCTAGGTCGGCGAGTCCTGGGAGAGGGTTGATGACGTCAACATTCG
GGCTCC
>H1_247-H1_246
(SEQ ID NO: 1175)
TGCGTAAAATACGCTTCTCGCAGATTATATAAGGTTCCTGTACCTAAAGACATTTCAAGGGTAGGGTGACTTCCC
ACAACACATAGCGACATGCAAATATAGGGTGTGTCTCCCCTGGCCCTTGTGGGCGTCTTCTCGCTAGGACGCACG
CGCGCTGCGTTTTCCCGCCTTCTGGCTCTAGGTCGGCGAGTCCCGGGAAAGGATTGATTACGTCAACATTCGGGC
TTC
>H1_248-H1_247
(SEQ ID NO: 1176)
TGCGTAAAAAAGGCTTCACGCAGATTATATAAGGTTCCTGTACCTAAAGACATTTCAAGGTTAGGGTGACTTCCC
ACAATACATAGCGACATGCAAATATAGGGGGGTGTGTCTCCCCTGGCCCTTGTGGGCGTCTTCTCGCTAGGACGC
ACGCGCGCTGCGTTTTCCCGCCTTGTGACTCTAGGTCGGCGAGTCCTGGGAAAGGATTGATTACGTCAACATTCG
GGCTTC
>H1_248-H1_249
(SEQ ID NO: 1177)
TGCGTAAAAAAGGCTTCACGGTGACTATATAAGGTTCCTGTACCTAATGACATTTCAAGATTAGGGTGACTTCCC
ACAATACATAGCGACATGCAAATAAAGGGGGGTTTCTCGTCTGTCCCCCCTGTGGGCGTCTTCTTGCTAGGACGC
ACGCGCGCTGCGTTTTCCCGCCTTGTGATTCTGGGTCGGCAAGTCCTGGGAAAGGATTGATTACGTCAACATTCG
GGCTTC
>H1_250-H1_251
(SEQ ID NO: 1178)
TGAGAAAAAAAGGCCACACGGAGAATATATAAGGCTCCCATATCTGAAGACATTTTAAGATTAGGGTGATTTCCC
ACAATACATAGCGACATGTAAATGTAGTGGGGCATGCCTTCCCTGTCCCTTGTGGGCAGCTTCTCGCCAGGACGC
ACGCGCGCTGCGTGTTCCCGCCTTGTGACTAAATTGGCGAGTCTGGGAGGAGACTGATGATGTCAGCATCATCAA
CTTTCCCGCTCC
>H1_251-H1_252
(SEQ ID NO: 1179)
TGAGGGAAGACTGTCGTAGGGAGAATATATAAGGCTCCCATATCGCTAGACATTTTAAGATGAGGGTGATTTCCC
ACAATGCATAGCGACATGTAAATGAAGTGGGGCATGCTTTCCCTGTCCCTTGTGGGCAGCTTCTCGCCAGGACGC
ACGCGCGCTGCGTGTTCCCGCCTTGTGACTAAATTGGCGAGTCTGGGAGGAGACTGATGATGTCAGCATCATCAA
CTTTCCCGCTCC
>H1_253-H1_242
(SEQ ID NO: 1180)
TGGGAAAAAAAGGCTTCACGCAGAATATATAAGGCTCCCATATCTAAAGACATTTCAAGGTTAGGGTGACTTCCC
ACAATACATAGCGACATGCAAATATAGCGGGGCGTGCCTCCCCTGTCCCTTGTGGGCATCTTCTCGCCAGGACGC
ACGCGCGCTGCGTGTTCCCGCCTTGTGACTCTAGGCTGGCGAGTCCCTGGGAGAGGGTTGATGACGTCAGCATCG
TCAACATTCGGGCTCC
>H1_253-H1_250
(SEQ ID NO: 1181)
TGAGAAAAAAAGGCCTCACGCAGAATATATAAGGCTCCCATATCTGAAGACATTTTAAGATTAGGGTGATTTCCC
ACAATACATAGCGACATGTAAATGTAGTGGGGCATGCCTCCCCTGTCCCTTGTGGGCAGCTTCTCGCCAGGACGC
ACGCGCGCTGCGTGTTCCCGCCTTGTGACTAAATTGGCGAGTCTGGGAGGAGATTGATGATGTCAGCATCATCAA
CTTTCCCGCTCC
>H1_253-H1_255
(SEQ ID NO: 1182)
CGCGAGAAAAATTCTTCACGCAGAATATATAAGGATCCCATATCTGAAGACATTTTACGATTACGGTGATTTCCC
ACAACACATAGCGACATGTAAATGTAGTGGGGCATGCCTCCCCTGTCCCTTGTGGGCAGCTTCTCGCCAGAACGC
ACGCGCGGTGCGTGTTCCCGCCTTGTGACTAAGTTGGCGAGTCAGGGAGGAGATTGATGATGTCATCATCGTCAG
CTCACCCGCTCC
>H1_253-H1_256
(SEQ ID NO: 1183)
CGAGAGAAAAAGTCTTCACGCAGAATATATAAGGATCCCATATCTGAAGACATTTTACGATTACGGTGATTTCCC
ACAACACATAGCGACATGTAAATGTAGTGGGGCATGCCTCCCCTGTCCCTTGTGGGCAGCTTCTCGCCAGAACGC
ACGCGCGGTGCGTGTTCCCGCCTTGTGACTAAGTTGGCGAGTCAGGGAGGAGATTGATGATGTCATCATCGTCAG
CTCACCCGCTCC
>H1_253-H1_257
(SEQ ID NO: 1184)
TGAGAAAAAAAGGCCTCACGCAGAATATATAAGGCTCCCATATCTGAAGACATTTTAAGGTTAGGGTGATTTCCC
ACAATACATAGCGACATGTAAATGTAGTGGGGCATGCCTCCCCTGTCCCTTGTGGGCAGCTTCTCGCCAGGACGC
ACGCGCGCTGCGTGTTCCCGCCTTGTGACTAAATTGGCGAGTCTGGGAGGAGATTGATGACGTCAGCATCATCAA
CTTTCCCGCTCC
>H1_253-H1_258
(SEQ ID NO: 1185)
TGAGAAAAAAAGGCCTCACGCAGAATATATAAGGCTCCCATATCTGAAGACATTTTAAGGTTAGGGTGATTTCCC
ACAATACATAGCGACATGCAAATATAGTGGGGCGTGCCTCCCCTGTCCCTTGTGGGCAGCTTCTCGCCAGGACGC
ACGCGCGCTGCGTGTTCCCGCCTTGTGACTAAATTGGCGAGTCTGGGAGGGGATTGATGACGTCAGCATCATCAA
CTTTCCCGCTCC
>H1_253-H1_261
(SEQ ID NO: 1186)
TGGGAAAAAGAGGGCTTCACGCAGAATATATAAGGCTCCCATATCTAAAGACATTTCACGGTTAGGGTGACTTCC
CCCACAATACATAGCGACATGCAAATATCATGGTCCTTCAGCGGGGCGTGCCTCCCCTGTCCCTTGTGGGCATCT
TCTCGCCAGGACACGCACGCGGCGCGCTGCGTGTTCCCGCCTTGTGACTTCTAGGCGGGCGAGTCCCTGGGAGAG
GGTTGGATGACGTCAGCATCGCCAACATTCGGGCTCC
>H1_253-H1_407
(SEQ ID NO: 1187)
TGGGAAAAAAAGGCTTCACGCAGAATATATAAGGCTCCCATATCTAAAGACATTTCAAGGTTAGGGTGACTTCCC
CCACAATACATAGCGACATGCAAATATCATGGTCCTTCAGCGGGGCGTGCCTCCCCTGTCCCTTGTGGGCATCTT
CTCGCCAGGACGCACGCGCGCTGCGTGTTCCCGCCTTGTGACTCTAGGCTGGCGAGTCCCTGGGAGAGGGTTGAT
GACGTCAGCATCGTCAACATTCGGGCTCC
>H1_261-H1_259
(SEQ ID NO: 1188)
CGGGAAAAAAACGGCTTCTGGTGGAAAATATATGAGGCCCATACCTGAAGACCTTTCACGGTTATGGTGACTTCC
CACAATACATAGCGACATGCAAATATAGTGGGGCGTGCCTCCACTGTCCTTTGCGGGCATCGTCTCGCCAGGAAG
CGCGCGCTGCGTGTTCCCGCCTTTTGACTCTAGGCGGGCGAATCCTGGGAGAGGGTTGTTGACGTCAACATTCGG
GCTCC
>H1_261-H1_260
(SEQ ID NO: 1189)
CAAGAGAAAACCGAGCCCTGCTGGAAAATATATGAGGCCCACTCTTCAAGACCTTTTATGGTTATGGTAACTTCC
CATAACACATAGCGACATGCAAATATCGTGGGGTGTGCCTCCACGGTCCTTTGCGGACACCGTCTTGCCCGTAAG
CGCGCTGGGTATTCCCGCCTTCTGACTCTAGGCGGGCGAATCCTAGGAGAGGGTTGTTGACGTCGACATTCGGGC
ACC
>H1_261-H1_264
(SEQ ID NO: 1190)
CAAGAGAGAAACGTGCCCTGCTGGAAAATATATGAGGCCCATTCCTCAAGACCTTTTATGGTTATGGTGACTTCC
CACAACACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_265
(SEQ ID NO: 1191)
CAAGAAAGAAACGTCCTCTGGTGGAAAATATATGAGGCCCATTCCTCAAGACCTTTTACGGTTATGGTGACTTCC
CACAACACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_268
(SEQ ID NO: 1192)
CAAGAAAGAAACGTCCTCTGGTGGAAAATATATGAGGCCCATTCCTCAAGACCTTTTACGGTTATGGTGACTTCC
CACAATACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_269
(SEQ ID NO: 1193)
CAAGAAAGAAACGTGCTCTGGTGGAAAATATATGAGGCCCATTCCTCAAGACCTTTTACGGTTATGGTGACTTCC
CACAACACATAGCGACATGCAAATATCGTGGGGTGTGCCTCCACTGTCCTTTGCGGACACCGTCTTGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_261-H1_270
(SEQ ID NO: 1194)
CGGGAAAAAAACGGCCTCTGGTGGAAAATATATGAGGCCCATACCTGAAGACCTTTCACGGTTATGGTGACTTCC
CACAATACATAGCGACATGCAAATATCGTGGGGCGTGCCTCCACTGTCCTTTGCGGGCATCGTCTCGCCCGGAAG
CGCGCGCTGTGTGTTCCCGCCTTTTGACTCTAGGCGGGCGAATCCTGGGAGAGGGTTGTTGACGTCAACATTCGG
GCTCC
>H1_261-H1_272
(SEQ ID NO: 1195)
TGGGAAAAAGAGGGCTTCACGCGGAATATATAAGGCTCCCATACCTAAAGACCTTTCACGGTTAGGGTGACTTCC
CCACAATACATAGCGACATGCAAATATAGTGGGGCGTGCCTCCCCTGTCCCTTGCGGGCATCTTCTCGCCAGGAC
ACGCGCGCGCCGCGCTGCGTGTTCCCGCCTTTTGACTTCTAGGCGGGCGAATCCTGGGAGAGGGTTGGATGACGT
CCAACATTCGGGCTCC
>H1_261-H1_292
(SEQ ID NO: 1196)
CGGGAAAAAAAGGGCTTCTGGCGGAAAATATATGAGGCCCATACCTGAAGACCTTTCACGGTTATGGTGACTTCC
CACAATACATAGCGACATGCAAATATAGTGGGGCGTGCCTCCCCTGTCCCTTGCGGGCATCTTCTCGCCAGGAAG
CGCGCGCGCTGCGTGTTCCCGCCTTTTGACTCTAGGCGGGCGAATCCTGGGAGAGGGTTGATGACGTCAACATTC
GGGCTCC
>H1_263-H1_271
(SEQ ID NO: 1197)
CAAGAGAGAAACTTGTCGTGCTGGAAAATATATGAGGCCCATTCCTCAGGACCTTTTATGGTTAGGGTGATTTCC
CACAATACATAGCGACATGCAAATATAGTGGGGTGTGCTTCCACTGTCCTTTGCGGACACCGTCTCGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_264-H1_263
(SEQ ID NO: 1198)
CAAGAGAGAAACTTGTCGTGCTGGAAAATATATGAGGCCCATTCCTCAGGACCTTTTATGGTTAGGGTGACTTCC
CACAACACATAGCGACATGCAAATATCGTGGGGTGTGCTTCCACTGTCCTTTGCGGACACCGTCTCGCCCGTAAG
CGCGCGCTGTGTATTCCCGCCTTTTGACTCTAGGCGGGCGAATCCTGGGAGAGGGTTGTTGACGTCGACATTCGG
GCTCC
>H1_266-H1_267
(SEQ ID NO: 1199)
CGAGGAAATAATCTCCCCTGGTGGCAAATATAGGAAGCCCATTCCTCAAGACCTTTTAAGGTTACGGTGACTTCC
CACAATACATAGCAACATGCAAATATTGTGGGGTGTGCCTTCACTGTCCTTTGCGGTCACTGTCTTGCCCATAAG
CGCGCTGTGTAATCCCGCCTTTTGACGTTAGGCAGGCGAATCCTGGGAGAGGGTTGCTGACGTCGACATTCGGCT
CC
>H1_268-H1_266
(SEQ ID NO: 1200)
CAAGGAAGTAACGTCCTCTGGTGGAAAATATATGAGGCCCATTCCTCAAGACCTTTTACGGTTATGGTGACTTCC
CACAATACATAGCAACATGCAAATATCGTGGGGTGTGCCTCCACTGTCCTTTGCGGACACTGTCTTGCCCGTAAG
CGCGCTGTGTAATCCCGCCTTTTGACTCTAGGCGGGCGAATCCTGGGAGAGGGTTGTTGACGTCGACATTCGGCT
CC
>H1_272-H1_273
(SEQ ID NO: 1201)
GGGGAGAAGGCGCTTTCCGCGGATTATATAAGGCTCCAGCACCTAGAGGCCTTTAACAGTTAGGGTGATTTCCCA
CAATGCATAGCGACATGCAAATATAGTTGGGTGTGCTTTCCCTGTTCCTTGCCTGCATCTTCTTGCCTGCGTGTT
CCCGCCTTTTGACTGCAGGCGGGCGAATCCTGGGAGAGAGTTGATGACGTCAACACTCAGGCTCC
>H1_272-H1_274
(SEQ ID NO: 1201)
GGGGAGAAAGGGGCTTCACGCGGAATATATAAGGCTCCCGTACCTAAAGGCCTTTCACGGTTAGGGTGACTTCCC
CACAATACATAGCGACATGCAAATATAGTTGGGCGTGCCTCCCCTGTCCCTTGCGGGCATCTTCTCGCCAGGACA
CGCGCGCGCCGCGCTGCGTGTTCCCGCCTTTTGACTTCCAGGCGGGCGAATCCTGGGAGAGGGTTGGATGACGTC
CAACATTCGGGCTCC
>H1_274-H1_291
(SEQ ID NO: 1202)
GGGGAGAAAGGGGCTTCACGGCGAATATATAAGGCTCCCGTACCTAAAGGCCTTTCACGGTTAGGGTGACTTCCC
CACAATACATAGCGACATGCAAATATAGTTGGGCGTGCCTCCCCTGTCCCTTGCGGGCATCTTCTCGCCCGGACA
CGCGCGCGCCGCGCTGCGTGTTCCCGCCTTTTGACTTCCAGGCGGGCGAATCCTGGGAGAGGGTTGGATGACGTC
CAACATTCGGGCTCC
>H1_276-H1_280
(SEQ ID NO: 1203)
AGGAAGGGAGCCTCACACGGCGGCTATATAAGGCCCCCTGCCCTGTAGGCCTTTCACAGTTAGGGCGACTTCCCC
ACAACACATAGCGACATGCAAATGTGGATGGGCGTGCCTCCCCGGTCCCTGCCGGCAACTTCTCTCCGGGACGCG
CGCTCGCGCTGAGTGTTCCCGCCTTTTGACGCCAGCGGAGCGAATCCGGGGAGCGGGCGGATGACGTCAACAGTG
CGGCTCC
>H1_279-H1_276
(SEQ ID NO: 1204)
AGGAAGGGAGCCTCACACGGCGGCTATATAAGGCCCCCTGCCCTGTAGGCCTTTCACAGTTAGGGCGACTTCCCC
ACAACACATAGCGACATGCAAATGTAGATGGGCGTGCCTCCCCGGTCCCTGCCGGCAACTTCTCTCCGGGACGCG
CGCTCGCGCTGAGTGTTCCCGCCTTTTGACGCCAGCCGAGCGAATCCGGGGAGCGGGCGGATGACGTCAACAGTG
CGGCTCC
>H1_280-H1_277
(SEQ ID NO: 1205)
AGGAAGGGAGCCTCACACGGCGGCTATATAAGGCCCCCTGCCCTGTAGGCCTTTCACAGTTAGGGCGACTTCCCC
ACAACACATAGCGACATGCAAATGTGGATGGGCGTGCCTCCCCGGTCCCTGCCAGCAACTTCTCTCCGGGACGCG
CGCTCGCGCTGAGTGTTCCCGCCTTTTGACGCCAGCGGAGCGAATCCGGGGAGCGGGCGGATGACGTGAACAGTG
CGGCTCC
>H1_282-H1_279
(SEQ ID NO: 1206)
GGGAAGAGAGCCTCACACGGCGGCTATATAAGGCCCCCTTACCTATAGGCCTTTTACGGTTAGGGTGACTTCCCC
ACAACACATAGCGACATGCAAATTTAGATGGGCGTGCCTCCCCTGTCCCTGTGGGCAACTTCTCTCCGGGACACG
CGCGCTCGCGCTGAGTGTTCCCGCCTTTTGACTTCCAGCCGAGCGAATCCTGGGAGAGGGCAGATGACGTCAACA
GTCAGGCTCC
>H1_282-H1_281
(SEQ ID NO: 1207)
GGGAAGAGGGCCTCACACGAGGGCTATATAAGGCCCCCTTACCTATAGGCCTTTTACGGTTAGAGTGACTTCCCA
CAACACCTAGCGACATGCAAATTTAGATGGGCGTGCCTCCTCTGTCCCTGTGGCAACACCTCTCCGGGACGCGCG
CTCGCTCTGAGTGTTCCCGCCTTTTGACTTCCAGCCGAACGAATCCTGGGAGAGGGCAGATGACGTCAATAGTCA
GGCTCC
>H1_282-H1_283
(SEQ ID NO: 1208)
GGGAAGAGGGCCTCACACGAGGGCTATATAAGGCCCCCTTACCTATAGGCCTTTTATGGTTAGAGTGACTTCCCA
CAACACCTAGCGACATGCAAATTTAGATGGGCGTGCCTCCTCTGTCCCTGTGGCAACACCTCTCCGGGACGCGCG
CTCGCTCTGAGCGTTCCCGCCTTTTGACTTCCAGCCGAACGAATCCTGGGAGAGGGCAGTGACGTCAATAGTCAG
GCTCC
>H1_282-H1_284
(SEQ ID NO: 1209)
GGGAAGAGAGCCTCACACGGCGGCTATATAAGGCCCCCTTACCTATAGGCCTTTTACGGTTAGGGTGACTTCCCA
CAACACATAGCGACATGCAAATTTAGATGGGCGTGCCTCCCCTGTCCCTGTGGGCAACTTCTCTCCGGGACACGC
GCGCTCGCGCTGAGTGTTCCCGCCTTTTGACTTCCAGCCGAGCGAATCCTGGGAGAGGGCAGATGACGTCAACAG
TCAGGCTCC
>H1_285-H1_282
(SEQ ID NO: 1210)
GGGAAGAGAGGCCTACACGGCGGCTATATAAGGCCCCCTTACCTATAGGCCTTTTACGGTTAGGGTGACTTCCCC
ACAACACATAGCGACATGCAAATTTAGATGGGCGTGCCTCCCCTGTCCCTGTGGGCAACTTCTCTCCGGGACACG
CGCGCTCGCGCTGAGTGTTCCCGCCTTTTGACTTCCAGCCGAGCGAATCCTGGGAGAGGGCAGATGACGTCAACA
GTCAGGCTCC
>H1_287-H1_285
(SEQ ID NO: 1211)
GGGAAGAGAGGCACTACACGGCGGCTATATAAGGCCCCCTTACCTATAGGCCTTTTACGGTTAGGGTGACTTCCC
CACAACACATAGCGACATGCAAATTTAGATGGGCGTGCCTCCCCTGTCCCTGTGGGCAACTTCTCTCCGGGACAC
GCGCGCTCCGCGCTGAGTGTTCCCGCCTTTTGACTTCCAGCCGAGCGAATCCTGGGAGAGGGCAGATGACGTCCA
ACAGTCAGGCTCC
>H1_287-H1_288
(SEQ ID NO: 1212)
GGGAGAAGGGGGAGTACACGGCGGATATATAAGGCCCCCTTATGTATAGTCCTTTTACGGTTAGGGTGACTTCCC
ACAACGCATAGCGACATGCAAATTTGACGGGCGTGCCTCCTCTGTCCCTGCGGGCAACTTCTCTCCTGGACGCGC
GCTCCGCGCTGCGTGTTCCCGCCTTTTGACTCCAGCCGAGCGAATCCTGGGAGAGGGCAGGTGACGTCCAACAGT
CAGGCTCG
>H1_287-H1_290
(SEQ ID NO: 1213)
GAGAGAGGCTGTGCACACGGCGGATATATAAGGCCCCCTTATGTATAATCCTTTACCGGTTAGGGTGACTTCCCA
CAACGCATAGCGACATGCAAATTTGACGGGCGTGCCTCCTCTGTCCCTGCGGGCAACTTCTCTCCTGGACGCGCG
CTCCGCGCTGCGTGTTCCCGCCTTTTGACTCCAGCCGAGCGAATCCTGGGAGAGGGCAGGTGACGTCCAACAGTC
AGGCTCG
>H1_288-H1_289
(SEQ ID NO: 1214)
GGGAGAAGGGGGAGTACACGGCGGATATATAAGGCCCCCTTATGTATAGTCCTTTTACGGTTAGGGTGACTTCCC
ACAACGCATAGCGACATGCAAATTTGACGGGCGTGCCTCCTCTGTCCCTGCGGGCAACTTCTCTCCTGGACGCGC
GCTCGCGCTGCGTGTTCCCGCCTTTTGACTCCAGCCGAGCGAATCCTGGGAGAGGGCAGGTGACGTCAACAGTCA
GGCTCG
>H1_291-H1_287
(SEQ ID NO: 1215)
GGGAAGAGAGGCACTACACGGCGGCTATATAAGGCCCCCTTACCTATAGGCCTTTTACGGTTAGGGTGACTTCCC
CACAACACATAGCGACATGCAAATTTAGATGGGCGTGCCTCCCCTGTCCCTTGTGGGCAACTTCTCTCCGGGACA
CGCGCGCTCCGCGCTGAGTGTTCCCGCCTTTTGACTTCCAGCCGAGCGAATCCTGGGAGAGGGCAGGATGACGTC
CAACAGTCAGGCTCC
>H1_294-H1_295
(SEQ ID NO: 1216)
TAGAAAAAATCGTAGTTTATGCTGGATTTATAAGATTCCCACATCTAAAGCCATTTCACAGTTACGGTGAACTTC
CCACTACACACGGCGATATGCAAATATAGCGGAAGTGTTCCTGAGGCGTGGTAAAGCGCGCGCGCGCTGAGAGTT
CCCGCCCTGTGGTGCTGGGCTGGAGATGCCTGAGAACTGGCTGATGACGGCAACGTTCGGGCTCC
>H1_295-H1_296
(SEQ ID NO: 1217)
TAGAAAAAATCGTGCCTATGCTGGATTTATAAGATTCCCACATCTAAAGCCATTTCTCAGTTACGGTGAACTTCC
CACTACACACGGCGATATGCAAATATAGCGGAAGTGTTCCTGAGGCGTGGTAAAGCGCGCGCGCGCTGAGAGTTC
CCGCCCTGTGGTGCTGGGCTGGAGATGCCTGAGAACTGGCTGATGACGGCAACGTTCGGGCTCC
>H1_296-H1_297
(SEQ ID NO: 1218)
TAGAAAAAATCGTGCCTACGCTGGATTTATAAGATTCCCACATCTAAAGCCATTTCTCAGTTACGGTGAACTTCC
CACTACACACGGCGATATGCAAATATAGCGGAAGTGTTCCTGAGGCGTGGTAAAGCGCGCGCGCGCTGAGAGTTC
CCGCCCTGTGGTGCTGGGCTGGAGATGCCTGAGAACTGGCTGATGACGGCAACGTTCGGGCTCC
>H1_298-H1_294
(SEQ ID NO: 1219)
TAGAAAAAATGGTAGTTTATGCGGGATTTATAAGACTCCCACATCTAAAGCCATTTCACAGTTACGGTGACTTCC
CCACAACACACGGCGATATGCAAATATAGCGGAAGTGTTCCTGAGGCGTGGTAAAGCGCACGCGCGCTGAGAGTT
CCCGCCCTGTGGTGCTGGGCCCGAGATGCCTGAGAGCGGGCTGATGACGGCAGCGTTTGGGCTCC
>H1_299-H1_298
(SEQ ID NO: 1220)
TAGAAAAAAGGGGAGTTTATGCGGGATTTATAAGACTCCCATATCTAAAGACATTTCACAGTTATGGTGACTTCC
CCACAACACATGGCGATATGCAAATATCGCGGAGCTGGCCCTGAGGCGTGGTAAGGCGCACGCGCGCTGAGAGTT
CCCGCCCTGTGGCGCTGGGCCCGAGATTCCTGAGAGCGGGTTGATGACGGCAGCGTTTGGGCTCC
>H1_299-H1_300
(SEQ ID NO: 1221)
TAGAGAAAAGGGGGTGTTTGCGGGATTTATAAGATTCCCATTGCTAAAGACATTTCACAGTTATGGTGACTTCCC
ACAACACTTGGCGATATGCAAATATCACGGAGTTGGCCCTGAGGCGCGGCGAGACGCACGCGCGCTGAGAGTTCC
CGCCTTCTCACCCTGGGTCCAAGGTTCCTGAAGGCGGGTTGAAGACTGCAGTGTTTGGGCGCC
>H1_301-H1_299
(SEQ ID NO: 1222)
TAGGAAAAAGGGGGGTTTATGCAGGATTTATAAGACTCCCATATCTAAAGACATTTCACGGTTATGGTGACTTCC
CCACAACACATAGCGATATGCAAATATCGCGGAGCGGGCCCTGAGGCGTGGTCAGGCGCACGCGCGCTGCGAGTT
CCCGCCCTGTGGCGCTGGGCCCGAGATTCCTGAGAGCGGGTTGATGACGTCAGCGTTTGGGCTCC
>H1_301-H1_302
(SEQ ID NO: 1223)
TAGGAAACGCGCATTTTAGGCAGGATTTATAAGACACCCATATCTAAAGACATTTCACGGTTATGGTGACTTCCC
ACAACACATAGCGAAATGCAAATATGTGGAGCAGGCGCTGAGGCGTGGTCGGGCGCACGCGCGCTGCGAGTTCCC
GCCCTTCGGCGCTAGGCCCGAGATGCCTGAGAGCTGGTTGATCACGTCTGCGTTTGGACTCA
>H1_301-H1_303
(SEQ ID NO: 1224)
TAGGAAAAGAGCATTTTAGGCAGGATTTATAAGACACCCATATCTAAAGACATTTCACGGTTATGGTGACTTCCC
ACAACACATAGCGAAATGCAAATATGTGGAGCGGGCGCTGAGGCGTGGTCGGGCGCACGCGCGCTGCGAGTTCCC
GCCCTTCGGCGCTAGGCCCGAGATTCCTGAGAGCTGGTTGATGACGTCAGCGTTTGGACTCC
>H1_304-H1_253
(SEQ ID NO: 1225)
TGGGAAAAAGAGGGGCTTCACGCAGCATTTATAAGGCTCCCATATCTAAAGACATTTCACGGTTAGGGTGACTTC
CCCCACAATACATAGCGACATGCAAATATCATGGTCCTTCAGCGGGGCGTGCCTCCCCCTGTCCCTTGGCCCGTG
GGCATCTTCTCGCCAGGACACGCACGCGGCGCGCTGCGTGTTCCCGCCTTGTGACTTCTAGGCGGGCGAGTCCCT
GGGAGAGGGTTGGATGACGTCAGCATCGCCAACATTCGGGCTCC
>H1_304-H1_293
(SEQ ID NO: 1226)
CGGGAAAAAGACGGGCCTCACGCCGCATTTATAAGGCTCCCATATCTAACGACATTTTACGGTTAGGGTGACTTC
CCACAATACATAGCGATATGCAAATATAGCGGGGCGTGTCTCCCCCTGGCCCTTGGCTCGTGGGCATCGTCTCGC
CAGGACGCATGCGCGCTGCTTGTTCCCGCCTTGACTACTTGCTAGTCCTGGGAGAGGGTTGATGACGTCAACGTT
CAGACTCC
>H1_304-H1_311
(SEQ ID NO: 1227)
CCGGCATAAGACGGGCCTCACGGCGCACTTATAAGGATCCCATATCTAACGACATTTTACGGTTAGGGTGACTTC
CCACAATACATAGCGATATGCAAATATAGCGGGGCGTGTCTACTCCTGGCCCTTGGTTTGTGGGCGTCGTCTCGC
CAGGACGCATGCGCACTGCTTGTTCCCGCCTTGACTACTTGCTAGTCCTGGGAGAGGGTTGATGACGTCAACGTT
CAGACTCC
>H1_306-H1_307
(SEQ ID NO: 1228)
TCAGCGTAAAGGAGTGCGTACAAAGAATTTATAAGGCTCGCATAGCTCTAGCTGCTTCACAGTTAGGGTGACTTC
CCACAAGCCATAGCGCATGTAAATATAAGGGCGTTTGTTCCCCCGCCCCCGTCCAGGCTGCAGCATCTCTCCAGG
ACGCAGGCGCACTGAGCCTTCCCGCCCGGTCACTCCAGACCCGCCATTCCCGGGCCAGGTTAATGACGTCACACT
TAAGCTCC
>H1_306-H1_310
(SEQ ID NO: 1229)
TCAGCGTAAAGGGATGCTTACGTAGAATTTATAAGGCTCCCATACCTAAAGCCATTTCACGGTTAGGGTGACTTC
CCACAAGACATAGCGACATGCAAATATAGAGGGGCGTGCTTCCCCTGTCCCGTCCCGTAGGCGTCTTCTCGCCAG
GGACGCACGCGCGCTGCGCCCTGTTCCCGCCCTGTCACTAGGGATTCTGGGCCGGCCATTCCCCGGGCGCAGGTT
GATGACGTCACGTTTGGGCTCC
>H1_308-H1_309
(SEQ ID NO: 1230)
TCAGCGTAAAAGAATGCTTAGCTAGAATTTATAAGGCTCCCAGACCTAAAGCCATATCTCGGTTAGGGTGACTTC
CCACAAGACATAGCGACATGCAAATATAGAGGGGGGGGCTTCCCCTGTGCCTTGTAGGCGTCTTCTCACGAAGTC
GCAAGCGCGTTGCGCCCTGTTCCCGCCCTGTCACTATTGATTATTGGCCGACCTTTCCTCGGGCGGAGTCTGATG
ACGTCATCGGTTCC
>H1_310-H1_308
(SEQ ID NO: 1231)
TCAGCGTAAAGGAATGCTTACCTAGAATTTATAAGGCTCCCAGACCTAAAGCCATATCACGGTTAGGGTGACTTC
CCACAAGACATAGCGACATGCAAATATAGAGGGGGGGGCTTCCCCTGTGCCTTGTAGGCGTCTTCTCACGAAGGA
CGCACGCGCGCTGCGCCCTGTTCCCGCCCTGTCACTATTGATTATTGGCCGACCATTCCCCGGGCGCAGTCTGAT
GACGTCATTCGGTTCC
>H1_312-H1_313
(SEQ ID NO: 1232)
TGGGGGAAGCTGGGCTCGATCAGCCTTTATAAAGCTCCAAAAACTCAAGACATTTTTCCGTTACGGTGGCTTCCC
ACAGTACACAGCGACATGCAAATAGCTTGCCAATGAATTCGCGGACCGCTTCCCGCCCCGGCGCAGGCGCGCGGA
CGCTGTCTCCCCTGGACGCGCGCTCGCGGTTCCCGGGAGCTGGCTGATGACGTTCGGTCTCC
>H1_312-H1_314
(SEQ ID NO: 1233)
TGGGGAAAGGTGGGCTCAAGCAGACTTTATAAAGCTCCAAAAACTCAAGACATTTTTCCGTTACGGTGGCTTCCC
ACAATACACAGCGACATGCAAATATAGTGGAGTGTGCTTGCCAATGATTTCCCGGGCCGCTTCTCGCCACGGCGC
AGGCGCGCTGTGTGTTCCCGCCCTGGACGGGCGCGCCCGCGGTTCCCGGGAGCGGGTTGATGACGTTCGGTCTCC
>H1_314-H1_315
(SEQ ID NO: 1234)
TGGGGAGTGGTGGATCCAAGCAGACTTTATAAAGCTCCGAAGGTCCAAGGCATCTTTCCCTTACGGTGGCTTCCC
ACAAGACATAGCGATATGCAAATTTATCGATACGTGCTTCAGACGCGCTTCTCGCCGCAGCGCAAGCGCGCTGTG
TGCTGACGCGGGGGACGGGCCAGTGCGCGATTCCCGGGAGCGGGTTGATGACGTTCGATCTCC
>H1_317-H1_316
(SEQ ID NO: 1235)
TGGGGAGAGGTGGATCCGAACAGACTTTATAAAGCTCCGAAAGCCCAAGGCATCTTTCCCTTACGGTAGCTTCCC
ACAAGACATAGCGACATGCAAATTTCTTGAAGTATGCTTCAGACGCGCTTCTCGCCACAGCGCAAGCGCGCTGTG
TGCTGACGCGGGAACGGGCCAGTGCGCGGTTCCCGGGAGCGGGTTGATGACGTTAGATCTCC
>H1_318-H1_317
(SEQ ID NO: 1236)
TGGGGAGAGGTGGATCCAAACAGACTTTATAAAGCTCCGAAAGCCCAAGGCATCTTTCCCTTACGGTGGCTTCCC
ACAAGACATAGCGACATGCAAATTTATTGAAGTATGCTTCAGACGCGCTTCTCGCCGCAGCGCAAGCGCGCTGTG
TGCTGACGCGGGAGACGGGCCAGTGCGCGGTTCCCGGGAGCGGGTTGATGACGTTCGATCTCC
>H1_322-H1_319
(SEQ ID NO: 1237)
TTCAGGGTGTAGACCGGCCGCCACTATAAGGCTCGAAAGAGGAATAAATTTTTCGTTTAGGGTGATTTCCCACAA
AGCACAGCGCGTAATTTGCATGTGCTCTACCCCAGGCTCCTGTGCTAGACAAGAAGCCCGCGCATCCGGGCAAGG
GATGATGACGTCGTCCTTCAAGAGCG
>H1_322-H1_321
(SEQ ID NO: 1238)
TTCAGGGTGTAGACCGGCCGCCACTATAAGGCTCGAAAGAGGAATAAATTTTTCGTTTAGGGTGATTTCCCACAA
AGCACAGCGCGTAATTTGCATGTGCTCTATCCCAGGCTCCTGTGCCAGACAAGAAGCCCGCGCATCCGGGCAAGG
GATGATGACGTCGTCCTTCAAGAGCG
>H1_322-H1_323
(SEQ ID NO: 1239)
TTCAGTGTGTAGACCGGCCGCCACTATAAGGTTCGAAAGAGGAATAAATTTTTCGTTTAGGGTGATTTCCCACAA
AGCACAGCGCGTAATTTGCATGTGCTCTACCCCAGGCTCCTGTGCTAGACAAGAAGCCCGCGCATCCGGGCAAGG
GATGATGACGTCGTCCTTCAAGAGCG
>H1_325-H1_327
(SEQ ID NO: 1240)
TGGAGGGTGTAGACCGGCCGCCACTATAAGGCTCGAAAGAGGAATAAATTTTTCGCTTACGGTGACTTCCCACAA
AGCACAGCGCGTAATTTGCATGTGCTCTATCCCAGGCTTCCTGTTCCAGACAAGAAGCCCGCGCATCCGGGCAAG
GGATGATGACGTCATCCCCGTCCTTCAAGCGCG
>H1_328-H1_329
(SEQ ID NO: 1241)
TGGAAGGTGGAGACCTGCCGCCATAATAAGACTCCAAAAGAGAGTGAATTTAACACTTACGGTGACTTCCCACAA
AGCACAGCGTGTAATTTGCATGCGCTCTAGCCCAGGCTCCAGCTCCGGACGAGAAGCCCGCGCATCCCGGCAAAG
GATGATGACGTCGTCCTTCAAGCGCT
>H1_328-H1_332
(SEQ ID NO: 1242)
TGGAGGGTGGAGACCGGCCACCATTATAAGACTCCAAAGCGGAATAAATTTTACGCTTATGGTGACTTCCCACAA
AGCACAGCGCGTAATTTGCATGTGCTCTATCCCAGGCTTCCTGCTCCAGACAAGAAGCCCGCGCATCCGGGCAAG
GGATGATGACGTCATCCCCGTCCTTCAAGCGCG
>H1_330-H1_328
(SEQ ID NO: 1243)
TGGAGGGTGGAGACCGGCCACCATTATAAGACTCCAAAGCGGAATAAATTTTACGCTTATGGTGACTTCCCACAA
AGCACAGCGCGTAATTTGCATGTGCTCTATCCCAGGCTTCCTGCTCCAGACAAGAAGCCCGCGCATCCGGGCAAG
GGATGATGACGTCATCCCCGTCCCTCAAGCGCG
>H1_332-H1_325
(SEQ ID NO: 1244)
TGGAGGGTGGAGACCGGCCACCATTATAAGACTCGAAAGCGGAATAAATTTTACGCTTATGGTGACTTCCCACAA
AGCACAGCGCGTAATTTGCATGTGCTCTATCCCAGGCTTCCTGCTCCAGACAAGAAGCCCGCGCATCCGGGCAAG
GGATGATGACGTCATCCCCGTCCTTCAAGCGCG
>H1_332-H1_333
(SEQ ID NO: 1245)
TACAGGGTGGAGATCGGCGAAAATTATAAGACTCGAAAGCGGCATAAAGTTTAAGCTTATGGTGACTTCCCACAA
AGCACAGCGCGTAATTTGCATGTGCTTTATCCCAGGCTCTTTCTCCAGACCAGTAGCCTGCACATCCGGGCAAGG
GGTGATGACGTCGTCCATCAAGCGCG
>H1_334-H1_330
(SEQ ID NO: 1246)
GGGAAGGTGGAGACCGGCCACCATTATAAGACTCCAAAGCGGAATACATTTTTCGGTTATGGTGACTTCCCACAA
AGCACAGCGCGTAATTTGCATGCGCTCTATCCCAGGCTTCCTGCTCCAGACAAGAAGCCCGCGCATCCGGGCAAG
GGATGATGACGTCATCCCCGTCCCTCAAGCGCG
>H1_335-H1_337
(SEQ ID NO: 1247)
ACGGCGGTGTGGAGGGCGAACTTTATAAGCCTCCGAAGAGAAAGCGATTTTTCAGTTATGGTGGTTTCCCACAAG
GCACAGCGCACAGTTTATTTGCATGCGCTCTAGCCCCGGCTCCCGCTCCAGACTAAGAAGCCCGCGCATTTCGGC
TGCGGATGATGACGTCGGGCCTCAAGCGCC
>H1_336-H1_335
(SEQ ID NO: 1248)
ACGGCGGTGTGGAGGGCGAACTTTATAAGCCTCCGAAGAGAAAGCGATTTTTCAGTTATGGTGGTTTCCCACAAG
GCACAGCGCACAGTTTATTTGCATGCGCTCCCGCCGCTTCTAGCCCCGGCTCCCGCTCCAGACTAAGAAGCCCGC
GCATTTCGGCTGCGGATGATGACGTCGGGCCTCAAGCGCC
>H1_338-H1_334
(SEQ ID NO: 1249)
GGGGAGGTGTGGGCCGGCCAGCTTTATAAGACTCCAAAGCGGAATGCATTTTTCAGTTATGGTGGCTTCCCACAA
GGCACAGCGCGCTGCTTATTTGCATGGGCTCACGCCGCTTCTAGCCCGGGCTTCCTGCTCCAGACTAAGAAGCCC
GCGCATCCCGGCCGGGCGAGGGATGATGACGTCATCCCCAGCCCTCAAGCGCG
>H1_338-H1_340
(SEQ ID NO: 1250)
GGAGGGCGGTGGCCGGCGAGCTTAATAAGCCTCGGAGGCGGGACGCCTGTTACAGTGACGGTGGTTTCCCACAAA
GCACGGCGCGGCGGTCTTGATTTGCATGCGCCTTTATGCCCGCCTCCCGCTCCGGAGAAGAAGCCCGCGCATCCC
GGCTGGGCTGGGGGTGATGACGTCAGGGCTCGAGCGCC
>H1_338-H1_342
(SEQ ID NO: 1251)
GGAGAGCGGTGGCCGGCGAGCTTAATAAGCCTCGGAAGCGGAACGCATTTTACAGTGATGGTGGTTTCCCACAAG
GCACAGCGCGGCGGCCTTTATTTGCATGCGCTTCTATTCCCGCCTCCCGCTCCAGAGAAGAAGCCCGCGCATCCC
GGCTCGGCTGGGGATGATGACGTCAGGGCTCGAGCGCC
>H1_338-H1_343
(SEQ ID NO: 1252)
GGGGTGGTGTGGCTGGCGAGCTTAATAAGGCTCCGAAGCGGAATGCATTTTACAGTGATGGTGGTTTCCCACAAG
GCACAGCGCGGCGTTTATTTGCATGCGCTTCTATTCCCGCCTCCCGCTCCAGACAAGAAGCCCGCGCATCCCGGC
TCGGCTGGGGATGATGACGTCAGGGCTCGAGCGCC
>H1_338-H1_344
(SEQ ID NO: 1253)
GGAGAGGGGTGGCCGGCGAGCTTAATAAGCCTCCGAAGCGGAACGCATTTTACAGTGATGGTGGTTTCCCACAAG
GCACAGCGCGGCGTTTATTTGCATGCGCTTCTATTCCCGCCTCCCGCTCCAGAGAAGAAGCCCGCGCATCCCGGC
TCGGCTGGGGATGATGACGTCAGGGCTCGAGCGCC
>H1_338-H1_345
(SEQ ID NO: 1254)
GGGGTGGTGTGGGTGGCGAGCTTTATAAGGCTCCGAAGCGGAATGCATTTTTCAGTTATGGTGGTTTCCCACAAG
GCACAGCGCGCCGTTTATTTGCATGGGCTCCCGCCGCTTCTAGCCCCGGCTCCCGCTCCAGACTAAGAAGCCCGC
GCATCCCGGCCCGGCTGGGGATGATGACGTCAGGCCTCAAGCGCC
>H1_338-H1_351
(SEQ ID NO: 1255)
GGGGAGGTGTGGGCGGCGAGCTTTATAAGACTCCAAAGCGGAATGCATTTTTCAGTTATGGTGGTTTCCCACAAG
GCACAGCGCGCTGCTTATTTGCATGGGCTCACGCCGCTTCTAGCCCGGGCTCCCGCTCCAGACTAAGAAGCCCGC
GCATCCCGGCCGGGCAGGGGATGATGACGTCAGCCCTCAAGCGCG
>H1_340-H1_341
(SEQ ID NO: 1256)
GCAAAGCGGTGGCCGGCGAGCTTAATAAGCCTCGGAGGCGGGACGCCTGTTACAGTGACGGTGGTTTCCCACAAA
GCACGGCGCGGCGGTCTTGATTTGCATGCGCCTTTATGCCCGCCTCCCGCTCCGGAGAAGAAGCCCGCGCATCCC
GGCTGGGCTGGGGGTGATGACGTCAGGGCTCGAGCGCC
>H1_346-H1_338
(SEQ ID NO: 1257)
GGGGAGGTGTGGGCCGGCCAGCTTTATAAGACTCCAAAGCGGAATGCATTTTTCAGTTATGGTGGCTTCCCACAA
GGCACAGCGCGCTGCTTATTTGCATGGGCTCACGCCGCTTCTAGCCCGGGCTTCCTGCTCCAGACTAAAGAAGCC
CGCGCATCCCGGCCGGGCGAGGGATGATGACGTCATCCCCAGCCCTCAAGCGCG
>H1_346-H1_347
(SEQ ID NO: 1258)
GGCGAGGGGTGGGCAGCCACCTTTATAAGACTCCAGAGCCGAATGCATTTCTCAGTTGTGGTGGCTTCCCATGAG
GCACAGCGCGCTATTTGCATGCGCTCTAGCCCGGGCTCCGGCTCTGGAATAAAAAATCCCGCGCATCCGGGTGAG
GGATGACGACGTCACCCTCAAGCGCT
>H1_349-H1_346
(SEQ ID NO: 1259)
GGGGAAGTGGGGGCAGGCCGGCTTTATAAGACTCCAGAGCGGAACGCATTTTTCAGTTATGGTGGCTTCCCACAA
GGCACAGCGCTATGCTTATTTGCATGGGCTCACGCCGCTTCTAGCCCGGGCCCCCTGCTCCAGACAAAAAAGCCC
GCGCATCCCGGCCGGGCGCGGGATGATGACGTCATCCCCAGCCCTCGAGCGCG
>H1_349-H1_348
(SEQ ID NO: 1260)
GAAGAAGTGGGGGAGACCGGCTTTATAAGACTCAGAAGGGAACAAACTTTTCAGTTGCGGTGGCTTCCCACAAGG
CACAGCGCTTTATTTGCATGCGCGCTAACCGGGGCCCCCTACTAAAAAGCCCGCGCATGCCCGGCGCGGGATGAT
GACGTCAGCCCTCGAGCGCG
>H1_349-H1_350
(SEQ ID NO: 1261)
GAAGTCGTGGGGGAGAGCGGCTTTATAAGACTCAGAAGGGAACAAACTTTTCAGTTGCGGTGGCTTCCCACAAGG
CACAGCGCTTTATTTGCATGCGCGCTAACCGGGGCCCCCTACTAAAAAGCCCGCGCATGTCCGGCGCGGGATGAT
GACGTCAGCCCCCGAGCGCG
>H1_352-H1_349
(SEQ ID NO: 1262)
GGGGAAGTGGGGGCAGGCCGGCTTTATAAGACTCCAGAGCGGAACGCATTTTTCAGTTATGGTGGCTTCCCACAA
GGCACAGCGCTATGCTTATTTCCATGGCCCCACCTCAGCATGGAAGCTCACGCCGCTTCTAGCCCGGGCCCCCTG
CTCCAGACAAAAAAGCCCGCGCATCCCGGCCGGGCGCGGGATGATGACGTCATCCCCAGCCCTCGAGCGCG
>H1_352-H1_354
(SEQ ID NO: 1263)
GGGAAGGCGGGGCCGGCGGCGCTAAAAGGCTCCGGGGCGGCCCGGACTTATCAGTTACGGTGGCTTCCCACGAGG
CGCAGCGCCGCTCATTTGCATGGCCCCACCCCAGACGGGAAGCCCGCGCCGCTCATTTGCGTGGCCCCGCCCCAG
ACGGGAAGCCCGCGCTGCTCGGCCGCGGTGGTGACGTCGGCCTCTCGCGCC
>H1_352-H1_356
(SEQ ID NO: 1264)
GGGAAAGCGGGGCCGGCGGCGCTAAAAGACTCCAGGGCGGCCCGGACTTATCAGTTACGGTGGCTTCCCACGAGG
CGCAGCGCCGCTCATTTGCATGGCCCCACCCCAGAAGGGAAGCCCGCGCCGCTCATTTGCGTGGCCCCGCCCCAG
ACGGGAAGCCCGCGCTGCCCGGCCGCGGTGGTGACGTCGGCCTCTCGCGCC
>H1_354-H1_355
(SEQ ID NO: 1265)
GGGAAGGCGGGGCCGGCGGCGCTAAAAGGCTCCGGGGCCGCCCGGACTTCACAGTTACGGTGGCTTCCCACGAGG
CGCAGCGCTGTCATTTGCATGGCCCCGCCCCAGACGGGAAGCCCGCGCTGCTCATTTGCGTGGCCCCGCCCCAGA
CGGGAAGCCCGCGCTGCTCGGCCGCGGTGGTGACGTCGGCCTCTCGCGCC
>H1_357-H1_358
(SEQ ID NO: 1266)
TGAAAGGGGCTCATCACAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTATGACAGAGACCCTGCGATTCCTGGGAGCTGGCTGATGACGTCAGTGTCTAACCTCC
>H1_357-H1_359
(SEQ ID NO: 1267)
TGAAAGGAACTCATCACAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTATGACAGAGACCCTGCGATTCCTGGGAGCTGGCTGATGACGTCAGTGTCTAACCTCC
>H1_357-H1_360
(SEQ ID NO: 1268)
TGAAAGGAACTCATCACAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTGTGACAGAGACCCTGCGATTCCTGGGAGCTGGCTGATGACGTCAGTGTCTAACCTCC
>H1_357-H1_363
(SEQ ID NO: 1269)
TGAAAGGAACTCATCTCAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTTCGCGCCGGCGCGC
TGCGTGGAGCGGAACTGTGACAGAGACCCTGCGATTCCTGGGAGCTGGCTGATGACGTCAGTGTCTAACCTCC
>H1_357-H1_365
(SEQ ID NO: 1270)
TGAGAGAAAATAAGCTCAAGCAGAACTTATAAGGCTCCCAAATGTACAGACATTTCTCGGTCATGGTAACTACCC
ACAACACACAGCGATATGCAAATATAGCAGAGTGTGCCTCCCCGCTCCCGTCCGGTCGTCTTCTCGCCGGAGCGC
AGGCGCGCTGCGTGGTGCGGGACTGTGACCCTGAGCCTGCGATTCCTGGGAGCGGGCTGATGACGTCAGCGTCTG
ACCTCC
>H1_357-H1_367
(SEQ ID NO: 1271)
TGAGAGAAACTAATCTCAAGCAGAACTTATAAGGCTCCCATATGTACAGACATTTCTCGGTCATGGTAACTACCC
ACAACACACAGCGATATGCAAATATAGCAGAGTGTGCCTCCCCGCTCGCGTCCGGTCGTCTTCTCGCCGGAGCGC
AGGCGCGCTGCGTGGTGCGGGACTGTGACCCTGAGCCTGCGATTCCTGGGAGCGGGCTGATGACGTCAGCGTCTA
ACCTCC
>H1_357-H1_368
(SEQ ID NO: 1272)
TGAGAGAAAGTAAGCTGAAGCAGAACTTATAAGGCTCCCAAATCTACAGACATTTCTCGGTCATGGTGACTACCC
ACAACACACAGCGATATGCAAATATCGCGGGGTGTGCCTCCCTGCTCTCGTCCGGTCGTCTTCTCGCCAGGGCGC
AGGCGCGCTGCGTGGTCCGGGCCTGTGACCCTGAGCCCGCGATTCCTGGGAGCGGGTTGATGACGTCAGCGTTTG
ACCTCC
>H1_357-H1_374
(SEQ ID NO: 1273)
TGGGAGAAAGTGGGCTGAAGCAGAACTTATAAGGCTCCCAAATCTAAAGACATTTTTCGGTCATGGTGACTTCCC
ACAACACACAGCGATATGCAAATATCGCGGGGTGTGCGCCTCCCTGCTCTCGTCCAGTCGTCTTCTCGCCAGGGC
GCACGCGTACTAGCGCGCTGCGTTGTTCCCGGCCTGTGACAGAGCCTGAGCCCGCGATTTCCTGGGAGCGGGTTG
ATGACGTCAGCGTTTGAACTCC
>H1_357-H1_395
(SEQ ID NO: 1274)
TGGGAGAAAGTGGGCTGAAGCAGAACTTATAAGGCTCCCAAATCTAAAGACATTTTTCGGTCATGGTGACTTCCC
ACAACACACAGCGATATGCAAATATCGCGGGGTGTGCGCCTCCCTGCTCTCGTCCAGTCGTCTTCTCGCCAGGGC
GCACGCGCGCTGCGTGTTCCCGGCCTGTGACCCTGAGCCCGCGATTCCTGGGAGCGGGTTGATGACGTCAGCGTT
TGAACTCC
>H1_363-H1_364
(SEQ ID NO: 1275)
TGAAAGGGACTCCTCTCAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTCGGCGCCGGCGCGC
TGCGTGGGGCGGAACTGTGACAGAGACCCTGCGATTCCTGGGAGCTGGCTGATGACGTCAGTGTCTAACCTCC
>H1_364-H1_361
(SEQ ID NO: 1276)
TGAAAGGGACTCCTCTCAAGCAGAGTTTATAAGGCTCCCATGTGTACAGCCATTTCTCGGTCATGGTAACTACCC
ACAACACACAGCGATATGCAAATATAGCAGAGAGTGTCTTCCCGCGCGCGCCTGGTCGTCTCGGCGCCGGCGCGC
TGCGTGGGGCGGAACTGTGACAGAGACCCTGCGATTCCTGGGAGCTGGCTGATGACATCAGTGTCTAACCTCC
>H1_365-H1_366
(SEQ ID NO: 1277)
TGAGGGAAGATAAGCTCAAGCAGAACTTATAAGGCTCCCAAATGTACAGACATTTATCGGTCATGGTAACTACCC
ACAACACACAGCGATATGCAAATATAGCAGAGCGTGCCTCCTGCACGGGCCGGTCGTCTTCTCGCCGGAGCGCAG
GCGCGCTGCGTGGTGCGGGACTGTGACCCTGAGCCTGCGATTCCTGGGAGCGGGCTGATGACGTCAGCGTCTGAG
CTCC
>H1_369-H1_396
(SEQ ID NO: 1278)
TGGGAGAAAGTGGGCTGAAGCAGGACTTATAAGGCTCCCAAATCTAAAGACATTTTTTGGTCATGGTGACTTCCC
ACAACACACAGCGTCATGCAAATATCATGGGGTGTGCGCCTCCCTGCTCCCGTCCAGTCGTCTTCTCGCCAGGGC
GCACGCGCGCTGCGTGTTCCCGGCCTGTGACCCTGAGCCCGCGATTGCTGGGAGCGAGTTGATGACGTCAGCGTT
TGAACTCC
>H1_371-H1_372
(SEQ ID NO: 1279)
TGGGGAAAGCTGGGCTCAAGCAGAGCTTATAAGGCTCTCGTACCTAAAGACATTTCACGGTCATGGTGACTACCC
ACAACACACAGCGACATGCAAATTTCGTGGAGTGTGCCTCCCTCCGCTTGTCCCGCGTCTTTTCTCTCCCGGGCG
CACGCGCGCACGCACGCGACGCGTTCCCGCCACAGCGCCCCCGCGGTTCCTGGGAGCGGGTTGATGACGTCAGCA
TTTGGACGCC
>H1_374-H1_373
(SEQ ID NO: 1280)
TGAAAGAAACTAGCCACAAACGGAAACTATAAGAGGTCCAAAGCTCAGTGTACTCTATGGTTAGGGTGACTTCCC
ACAATACATAGCGATATGCAGATTTCTTCCCCAATCTGGCCCGCCGGGCCCTCCCTAGAGCGCATGCGCTGCAGG
TCCACGGCAGAGCACTGGGCGGGCGATCCCGGGAGCGGGTTGATGACGTCAGCGTTTGAACTCC
>H1_374-H1_375
(SEQ ID NO: 1281)
TGAAAGAAACTAGCCACAAACGGAAACTATAAGAGGTCCAAAGCTCAGTGTACTCTATGGTTAGGGTGACTTCCC
ACAATACATAGCGATATGCAGATTTCTTCCCCAGTCTGGCCCGCTGGGCCCTCCCTAGAGCGCATGCGCTGCAGG
TCCACGGCAGAGCACTGGGCGGGCGATCCCGGGAGCGGGTTGATGACGTCAGCGTTTGAACTCC
>H1_374-H1_376
(SEQ ID NO: 1282)
TGAAAGAAACTAGTTACAAACGGAAACTATAAGAGGTCCAAAGCTCAGTGTACTTTATGGTCAGGGTGACTTCCC
ACAATACATAGCGATATGTAGATTTCTTCCCCGATCTGGGCCCGCCGGGTCCTCCCTAGAGCGCATGCGCTGCAG
GTCCACGGCAGAGGACTGGGCGGGCGATTCCCGGGAGCGGGTTGATGACGTCAGCGTTTGAACTCC
>H1_374-H1_391
(SEQ ID NO: 1283)
TGAGAGAAAATGGTTTGAAGCAGAACTTATAAGAATCCCAAATCTCAGTATATTTTTTGGTCATGGTGACTTCCC
ACAATACACAGCGATATGTAGATATCGCGGGGAGCACCTCCCAGTTCTGGTCCAGTCGGCTCCTCGCTAGGGCGC
ACGCGTACTAGCGCGCTGCATGGTTCCCGGCCTGTGACAGTGCCTGAGCCCGCGATTTCCTGGGAGCGAGTTGAT
GACGTCAGCGTTTGAACTCC
>H1_374-H1_392
(SEQ ID NO: 1284)
TGAAAGAAACTGGTTTCAAACGGAAACTATAAGAGGTCCAAATCTCAGTATACTTTTTGGTCAGGGTGACTTCCC
ACAATACACAGCGATATGTAGATTTCCTCCCCGATCTGGTCCCGTCGGCTCCTCGCTAGGGCGCATGCGCTGCAG
GTCCCCGGCCTATGACTGGGCCGGCGATTTCCCGGGAGCGAGTTGATGACGTCAGCGTTTGAACTCC
>H1_377-H1_378
(SEQ ID NO: 1285)
TGAAAAAAAAGGTTTCAAAGCTACACTTATAAGGCTCCCAAATGTCAGTATATTTTTTGGTCACGGTGACTTCCC
ACAATGCATAGCGATATGTAGATATTGCGAGGAGTACCTCCCAGTTCTGGTCCTGTCAGCTCTTTGCTAGGACGC
ACGCGCTGCAGGTTCCCAGCCTGTGATTGGGCCAGCGATTCCGGGAGCGAATTGATGACGTCAGCGTTTGAACTC
C
>H1_377-H1_380
(SEQ ID NO: 1286)
TGAAAAAAAAGGTTTCAAAGCTACACTTATAAGGCTCCCAAATCTCAGTATATTTTTTGGTCACGGTGACTTCCC
ACAATGCATAGCGATATGTAGATATTGCGAGGAGTACCTCCCAGTTCTGGTCCTGTCAGCTCTTTGCTAGGACGC
ACGCGCTGCAGGTTCCCAGCCTGTGATTGGGCCAGCGATTCCGGGAGCGAATTGATGACGTCAGCGTTTGAACTC
C
>H1_383-H1_377
(SEQ ID NO: 1287)
TGAAAGAAAAGGTTTCAAAGCTACACTTATAAGGATCCCAAATCTCAGTATATTTTTTGGTCACGGTGACTTCCC
ACAATACACAGCGATATGTAGATATCGCGAGGAGTACCTCCCAGTTCTGGTCCTGTCAGCTCTTTGCTAGGGCGC
ACGCGCTGCAGGTTCACAGCCTGTGATTGGGCCCGCGATTCCGGGAGCGAATTGATGACGTCAGCGTTTGAACTC
C
>H1_383-H1_384
(SEQ ID NO: 1288)
TGAAAGAAAAGGTTTCAAAGCTACACTTATAAGGATCCCAAATCTCAGTATATTTTTTGGTCACGGTGACTTCCC
ACAAGACACAGCGATATGTAGATATCGCGAGGAGTACCTCCCAGTTCTGGTCCTGTCAGCTCTTTGCTAGGGCGC
ACGCGCTGCAGGTTCACAGCCTGTGATTGGGCCCGCGATTCCGGGAGCGAATTGATGACGTCAGCGTTTGAACTC
C
>H1_386-H1_383
(SEQ ID NO: 1289)
TGAAAGAAAAAGTTTTGAAGCAGAACTTATAAGAATCCCAAATCTCAGTATATTTTTTGGTCATGGTGACTTCCC
ACAATACACAGCGATATGTAGATATCGCGAGGAGCACCTCCCAGTTCTGGTCCTGTCAGCTCCTCGCTAGGGCGC
ACGCGCGCTGCATGGTTCACAGCCTGTGACCCTGGGCCCGCGATTCCTGGGAGCGAGTTGATGACGTCAGCGTTT
GAACTCC
>H1_386-H1_385
(SEQ ID NO: 1290)
TGAAAGCAAAAGTTTTGAAGCAGAACTTATAAGAAGCCCAAATCTCAGTATATTTTTTGGTCATGGTGACTTCCC
ACAATACACAGCGATATGTAGATATCGCGAGGAGCACCTCCCAGTTCTGGTCCTGTCAGCTCCTCACTAGGGCGC
ATGCGCGCTGCATGGTTCACAGCCTGTGACCCTGGGCCTGCGATTCCTGGGAGCGAGTTGATGACGTCAGCGTTT
GAACTCC
>H1_386-H1_387
(SEQ ID NO: 1291)
TGAAAGCAAAAGTTTTGAAGCAGAACTTATAAGAAGCCCAAATCTCAGTATATTTTTTGGTCATGGTGACTTCCC
ACAATACACAGCGATATGTAGATATCGCGAGGAGCACCTCCCAGTTCTGGTCCTGTCAGCTCCTCACTAGGGCGC
ATGCGCTGCAGGTTCACAGCCTGTGACTGGGCCTGCGATTCCTGGGAGCGAGTTGATGACGTCAGCGTTTGAACT
CC
>H1_388-H1_386
(SEQ ID NO: 1292)
TGAGAGAAAATGTTTTGAAGCAGAACTTATAAGAATCCCAAATCTCAGTATATTTTTTGGTCATGGTGACTTCCC
ACAATACACAGCGATATGTAGATATCGCGGGGAGCACCTCCCAGTTCTGGTCCAGTCGGCTCCTCGCTAGGGCGC
ACGCGTACTAGCGCGCTGCATGGTTCCCGGCCTGTGACAGTGCCTGAGCCCGCGATTCCTGGGAGCGAGTTGATG
ACGTCAGCGTTTGAACTCC
>H1_388-H1_390
(SEQ ID NO: 1293)
TGAGAGAAAATGTTTTGAAGCAGAACTTATAAGAATCCCAAATCTCAGTATATTTTTTGGTCATGGTGACTTCCC
ACAATACACAGCGATATGTAGATATGGTGGGGAGCACCTCCCAGTTCTGGCCCAGTCGGCTCCTCGCTAGGGCGC
ACGCGTACTAGCGCGCTGCGGGTTCCCGGCCTGTGACAGTGCCTGAGCCCGCGATTCCTGGGAGCGAGTTGATGA
CGTCAGCGTTTGAACTCC
>H1_388-H1_393
(SEQ ID NO: 1294)
TAAGAGAAAGTTTTTTGAAGCAGAACTTATAAGGATCCCAAAACTCAGTATATTTTTTGGTCATGGTGACTTCCC
ACAATACACAGCGATATGTAGATATGGTGGGGAGCACCTCCCAGTTCTGGCCCAGTCGGCTCCTCGCTAGGGCGC
ACGCGTACTAGCGCGCTGCGGGTTCCCGGCCTGTGACAGTGCCTGAGCCCGCGATTCCTGGGAGCGAGTTGATGA
CGTCAGCGTTTGAACTCC
>H1_391-H1_388
(SEQ ID NO: 1295)
TGAGAGAAAATGGTTTGAAGCAGAACTTATAAGAATCCCAAATCTCAGTATATTTTTTGGTCATGGTGACTTCCC
ACAATACACAGCGATATGTAGATATCGCGGGGAGCACCTCCCAGTTCTGGTCCAGTCGGCTCCTCGCTAGGGCGC
ACGCGTACTAGCGCGCTGCATGGTTCCCGGCCTGTGACAGTGCCTGAGCCCGCGATTCCTGGGAGCGAGTTGATG
ACGTCAGCGTTTGAACTCC
>H1_393-H1_394
(SEQ ID NO: 1296)
TAAGAGAAAGCTTTCTGAACCAGAGCTTATAAAGATCCCAAAACTCAGGCTATATTTTGGTCATGGTGACTTCCC
ACAATACACAGCGATATGTAGATATAGTGGGGAGCACCTCCCAGTTCTGGCCCAGTCGGGTCCTCTCTAGGGCGC
ACGCGCGCTGCGGGTTCCCGGCCTGTGACAGTGCCTGAGCCCGCGATTCCTGGGAGCGAGTTGACGTCACCGTTT
GAACTTC
>H1_395-H1_369
(SEQ ID NO: 1297)
TGGGAGAAAGTGGGCTGAAGCAGAACTTATAAGGCTCCCAAATCTAAAGACATTTTTCGGTCATGGTGACTTCCC
ACAACACACAGCGATATGCAAATATCATGGGGTGTGCGCCTCCCTGCTCTCGTCCAGTCGTCTTCTCGCCAGGGC
GCACGCGCGCTGCGTGTTCCCGGCCTGTGACCCTGAGCCCGCGATTGCTGGGAGCGAGTTGATGACGTCAGCGTT
TGAACTCC
>H1_398-H1_357
(SEQ ID NO: 1298)
TGGGAAAAAGTGGGGCTCAAGCAGAATTTATAAGGCTCCCAAACCTAAAGACATTTTACGGTTATGGTGACTTCC
CACAACACACAGCGACATGCAAATATCGCGGGGTGTGCGGCCTCCCTGCTCTCGTCCAGGCGTCTTCTCGCCAGG
GCGCACGCGCGCACGCGCGCTGCGCTGTTCCCGCCCTGGTGACGGAGCCTGAGCCCGCGATTTCCTGGGAGCGGG
TTGATGACGTCAGCGTTTGGACTCC
>H1_398-H1_399
(SEQ ID NO: 1299)
CAGGAAAGACTGCGCTGAGGCAGACTTTATAAGGCTCCCGCGCAGAAAGAAACTTTATAGTTATGGTGATTTCCC
ACAAGCCACTGCGTCATGCAAATAAAGCAGGGTTGACGGCTTCCAAGTATGTACCTTAAGGTTTTTCTCTAGGCC
GCGTACGCTCTGCGTATTCAGCCACGTGACCCTGAGCCAGTGGTTGTTGGGAGCACGTTGTGGACCTCTGCGTTT
GGATTCC
>H1_398-H1_400
(SEQ ID NO: 1300)
CAGGAAAGAGTGGGGCTCAGGCAGACTTTATAAGGCTCCCAAACAGAAAGACACTTTACAGTTATGGTGACTTCC
CACAAGACACTGCGTCATGCAAATATCGCAGGGTTGGCGGCCTTCCTTCTATCTTCCTTAAGGTTTCTCTCTAGG
GCGCGTACGCGCTGCGTATTCCCGCCCCGGTGACCCTGAGCCAGTGGTTGTTGGGAGCACGTTGATGACGTCTGC
GTTTGGATTCC
>H1_402-H1_403
(SEQ ID NO: 1301)
TGGGGAGTGGCCGCCTAGGGGGCGATATATAAGGCTCACAAAACCCGTGCTATTTCTTACAGAGGGTGAATATCC
CCATGATCCTCGGCGGCATGCAAATAATAGTTGCGTCAGAGTAGAGCGCAGCCTGCCGGTCTCTCCTAGCGCGGG
AAATCCTGTTTTCTTCTTCAGTCCCGGTGACGAGGACGCGCGCGCGCACCGTAGCCGGACAACGGTCTGGTAAGG
TAGGCGGGATTCGGTTGAGAGCGCC
>H1_403-H1_404
(SEQ ID NO: 1302)
CGTGGAATCCCCGCCTAGGGGGCGCTATATAAGGCTCACCAAACCCGTGCTATTTCTTACAGAGGGTGAATATCC
CATGATCCTTGGCGGCATGCAAATAACAGCTTGCGTCAGAGTAGAGCGCAGCCTACCAGTCTTTCCTAGCGCGGG
AAATCCCGTTTTCTTCTGAGGTCGCCGGTGACGCGCGCGTGCGCCGTAGCCAGAGAACGGTCCGGGAAGGTAGGC
CGGCCGGGATTCGGTTGAGAGCGCC
>H1_407-H1_408
(SEQ ID NO: 1303)
TGGGACAAAAAACTCTTGGTCACATTATATAAGAATCCCATATCTAAAGACATTTCAGGGTTAGGGTGACTTCCC
CAACAATACATAGCGACATGCAAATATCATGGTCCTTCCAGGAGGCGTGCCTCCCCGTCCCCTTGGTCCAGGTCT
TGCTGGGGCGCACGCGCGCTGCGTGTTCCCGCTCTGTGACTCTCAGCTCGCGATTCCTGAGAGCGGATTGGTGAA
GTCAATGTTCTGGCTCC
>FIG. 17 Consensus Sequence
(SEQ ID NO: 1868)
TGAGCTTCCCTCCGCCCTATGRGRAARRGTGGTYCYAYNCAGAACTTATAAGRYTCCCAWAYYYAAAGACATTTC
WCGWTTATGGTGAYTTCCCAGAABACAYAGCGACATGCAAATATTGYAGGGCGTSMCWCCCCTGTCCCTNACRGY
CRTCTTCCTGCCAGGGCGCACGCGCGCTGSGTGTTCCCGCSTAGTGACDCTGGGCCCGCGATTCCTTGGAGCGGG
TTGATGACGTCAGCGTTCGAATTCCATGGCG

What is claimed is:

1. A non-naturally occurring nuclease system comprising a vector comprising a compact bidirectional promoter, wherein the compact bidirectional promoter comprises: a) at least one regulatory element that provides for transcription in one direction of at least one nucleotide sequence encoding a guide RNA (gRNA), wherein the gRNA hybridizes with a target sequence of a nucleic acid; and b) at least one regulatory element that provides for transcription in the opposite direction of a nucleotide sequence encoding a nuclease, wherein the gRNA targets and hybridizes with the target sequence and directs the nuclease to the nucleic acid, wherein the bidirectional promoter is not one or more of SEQ ID NO: 70-SEQ ID NO: 106 and SEQ ID NO: 241-SEQ ID NO: 255.

2. The system of claim 1, wherein the compact bidirectional promoter is between 50 and 225 bp.

3. The system of claim 1, wherein the compact bidirectional promoter is between 50 and 200 bp.

4. The system of claim 1, wherein the compact bidirectional promoter is between 50 and 180 bp.

5. The system of any preceding claim, wherein the bidirectional promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

6. The system of any preceding claim, wherein the compact bidirectional promoter comprises an H1 promoter.

7. The system of claim 6, wherein the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to the H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

8. The system of any one of claims 1-5, wherein the compact bidirectional promoter comprises a Gar1 promoter.

9. The system of claim 8, wherein the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto.

10. The system of claim 8 or 9, wherein the Gar1 promoter is a human Gar1 promoter.

11. The system of any one of claims 1-5, wherein the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

12. The system of any preceding claim, wherein the compact promoter comprises at least 95%, 98%, 99%, 99.5% or 100% identity to a naturally-occurring mammalian promoter.

13. The system of any preceding claim, wherein the target sequence comprises the nucleotide sequence AN₁₉NGG, GN₁₉NGG, CN₁₉NGG, or TN₁₉NGG.

14. The system of any preceding claim, wherein the nuclease is a nuclease-dead nuclease.

15. The system of any preceding claim, wherein the nuclease is an RNA-directed nuclease.

16. The system of claim 15, wherein the RNA-directed nuclease is a Cas protein.

17. The system of claim 16, wherein the Cas protein is codon optimized for expression in the cell and/or is a Type-II Cas protein or a Type V Cas protein.

18. The system of claim 17, wherein the cell is a eukaryotic cell.

19. The system of claim 18, wherein the eukaryotic cell is a mammalian cell (e.g. a human cell).

20. The system of any preceding claim, wherein the system is packaged into a single vector.

21. The system of claim 20, wherein the single vector is a viral vector or a plasmid.

22. An expression construct comprising the system of any preceding claim.

23. A vector comprising the expression construct of claim 22.

24. The vector of claim 23, wherein the vector comprises an adeno-associated viral (AAV) vector.

25. A method, the method comprising introducing into a cell a non-naturally occurring nuclease system comprising a vector comprising a compact bidirectional promoter, wherein the compact bidirectional promoter comprises: a) at least one regulatory element that provides for transcription in one direction of at least one nucleotide sequence encoding a guide RNA (gRNA), wherein the gRNA hybridizes with a target sequence of a nucleic acid molecule; and b) at least one regulatory element that provides for transcription in the opposite direction of a nucleotide sequence encoding a nuclease, wherein the gRNA targets and hybridizes with the target sequence and directs the nuclease to the nucleic acid molecule, wherein the bidirectional promoter is not one or more of SEQ ID NO: 70-SEQ ID NO: 106 and SEQ ID NO: 241-SEQ ID NO: 255.

26. The method of claim 25, wherein the compact bidirectional promoter is between 50 and 225 bp.

27. The method of claim 25, wherein the compact bidirectional promoter is between 50 and 200 bp.

28. The method of claim 25, wherein the compact bidirectional promoter is between 50 and 180 bp.

29. The method of any one of claims 25-28, wherein the bidirectional promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

30. The method of any one of claims 25-29, wherein the compact bidirectional promoter comprises an H1 promoter.

31. The method of claim 30, wherein the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to the H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

32. The method of any one of claims 25-29, wherein the compact bidirectional promoter comprises a Gar1 promoter.

33. The method of claim 32, wherein the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto.

34. The method of claim 32 or 33, wherein the Gar1 promoter is a human Gar1 promoter.

35. The method of any one of claims 25-29, wherein the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

36. The method of one of claims 25-35, wherein the compact promoter comprises at least 95%, 98%, 99%, 99.5% or 100% identity to a naturally-occurring mammalian promoter.

37. The method of any one of claims 25-36, wherein the target sequence comprises the nucleotide sequence AN₁₉NGG, GN₁₉NGG, CN₁₉NGG, or TN₁₉NGG.

38. The method of any one of claims 25-37, wherein the nuclease is a nuclease-dead nuclease.

39. The method of any one of claims 25-38, wherein the nuclease is an RNA-directed nuclease.

40. The method of claim 39, wherein the RNA-directed nuclease is a Cas protein.

41. The method of claim 40, wherein the Cas protein is codon optimized for expression in the cell and/or is a Type-II Cas protein or a Type-V Cas protein.

42. The method of claim 41, wherein the cell is a eukaryotic cell.

43. The method of claim 42, wherein the eukaryotic cell is a mammalian cell (e.g., a human cell).

44. The method of any one of claims 25-43, wherein the system is packaged into a single vector.

45. The method of claim 44, wherein the single vector is a viral vector or a plasmid.

46. A non-naturally occurring nuclease system comprising a vector comprising a compact bidirectional promoter, wherein the compact bidirectional promoter comprises both RNA pol II and RNA pol III activity, wherein a) the promoter provides for transcription of at least one nucleotide sequence encoding a guide RNA (gRNA), wherein the gRNA hybridizes with a target sequence of a nucleic acid; and b) the promoter provides for transcription of a nucleotide sequence encoding a nuclease, wherein the gRNA targets and hybridizes with the target sequence and directs the nuclease to the nucleic acid, wherein the bidirectional promoter is not one or more of SEQ ID NO: 70-SEQ ID NO: 106 and SEQ ID NO: 241-SEQ ID NO: 255.

47. The system of claim 46, wherein the compact bidirectional promoter is between 50 and 225 bp.

48. The system of claim 46, wherein the compact bidirectional promoter is between 50 and 200 bp.

49. The system of claim 46, wherein the compact bidirectional promoter is between 50 and 180 bp.

50. The system of any preceding claim, wherein the bidirectional promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

51. The system of any preceding claim, wherein the compact bidirectional promoter comprises an H1 promoter.

52. The system of claim 51, wherein the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to the H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

53. The system of any one of claims 46-50, wherein the compact bidirectional promoter comprises a Gar1 promoter.

54. The system of claim 53, wherein the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto.

55. The system of claim 53 or 54, wherein the Gar1 promoter is a human Gar1 promoter.

56. The system of any one of claims 46-50, wherein the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

57. The system of any one of claims 46-56, wherein the compact promoter comprises at least 95%, 98%, 99%, 99.5% or 100% identity to a naturally-occurring mammalian promoter.

58. The system of any one of claims 46-57, wherein the target sequence comprises the nucleotide sequence AN₁₉NGG, GN₁₉NGG, CN₁₉NGG, or TN₁₉NGG.

59. The system of any one of claims 46-58, wherein the nuclease is a nuclease-dead nuclease.

60. The system of any one of claims 46-59, wherein the nuclease is an RNA-directed nuclease.

61. The system of claim 60, wherein the RNA-directed nuclease is a Cas protein.

62. The system of claim 61, wherein the Cas protein is codon optimized for expression in the cell and/or is a Type-II Cas protein or a Type V Cas protein.

63. The system of claim 62, wherein the cell is a eukaryotic cell.

64. The system of claim 63, wherein the eukaryotic cell is a mammalian cell (e.g. a human cell).

65. The system of any one of claims 46-64, wherein the system is packaged into a single vector.

66. The system of claim 65, wherein the single vector is a viral vector or a plasmid.

67. An expression construct comprising the system of any one of claims 46-66.

68. A vector comprising the expression construct of claim 67.

69. The vector of claim 68, wherein the vector comprises an adeno-associated viral (AAV) vector.

70. A method, the method comprising introducing into a cell a non-naturally occurring nuclease system comprising a vector comprising a compact bidirectional promoter, wherein the compact bidirectional promoter comprises both RNA pol II and RNA pol III activity, wherein a) the promoter provides for transcription of at least one nucleotide sequence encoding a guide RNA (gRNA), wherein the gRNA hybridizes with a target sequence of a nucleic acid; and b) the promoter provides for transcription of a nucleotide sequence encoding a nuclease, wherein the gRNA targets and hybridizes with the target sequence and directs the nuclease to the nucleic acid, wherein the bidirectional promoter is not one or more of SEQ ID NO: 70-SEQ ID NO: 106 and SEQ ID NO: 241-SEQ ID NO: 255.

71. The method of claim 70, wherein the compact bidirectional promoter is between 50 and 225 bp.

72. The method of claim 70, wherein the compact bidirectional promoter is between 50 and 200 bp.

73. The method of claim 70, wherein the compact bidirectional promoter is between 50 and 180 bp.

74. The method of any one of claims 70-73, wherein the bidirectional promoter comprises a nucleic acid sequence selected from SEQ ID NOs: 107-255 or the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to an H1 promoter (e.g., from about nucleotide 20 to about nucleotide 490 as numbered in FIG. 3), or a nucleic acid sequence having at least about 80%, at least about 85%, at least about 90%, at least about 95%, at least about 98%, at least about 99%, or at least about 99.5% identity thereto.

75. The method of any one of claims 70-74, wherein the compact bidirectional promoter comprises an H1 promoter.

76. The method of claim 75, wherein the H1 promoter is selected from the portion of any one of SEQ ID NOs: 25-106, 469-476, 559-564, 609-614, 673-678, 681, 692-697, 706-711, 719-724, 729-734, 748-753, 784-789, 904-909, 920-925, 936-1303, or any sequence in FIGS. 3-19 that corresponds to the H1 promoter (e.g., from about nucleotide 20 to about nucleotide 490 as numbered in FIG. 3), or a promoter having at least about 80%, at least about 85%, at least about 90%, at least about 95%, at least about 98%, at least about 99%, or at least about 99.5% identity thereto.

77. The method of any one of claims 70-74, wherein the compact bidirectional promoter comprises a Gar1 promoter.

78. The method of claim 77, wherein the Gar1 promoter is selected from SEQ ID NOs: 107-203, or a promoter having at least about 80%, at least about 85%, at least about 90%, at least about 95%, at least about 98%, at least about 99%, or at least about 99.5% identity thereto.

79. The method of claim 77 or 78, wherein the Gar1 promoter is a human Gar1 promoter.

80. The method of any one of claims 70-74, wherein the compact promoter comprises a bidirectional promoter selected from SEQ ID NOs: 204-255, or a promoter having at least about 80%, at least about 85%, at least about 90%, at least about 95%, at least about 98%, at least about 99%, or at least about 99.5% identity thereto.

81. The method of one of claims 70-80, wherein the compact promoter comprises at least 95%, 98%, 99%, 99.5% or 100% identity to a naturally-occurring mammalian promoter.

82. The method of any one of claims 70-81, wherein the target sequence comprises the nucleotide sequence AN₁₉NGG, GN₁₉NGG, CN₁₉NGG, or TN₁₉NGG.

83. The method of any one of claims 70-82, wherein the nuclease is a nuclease-dead nuclease.

84. The method of any one of claims 70-83, wherein the nuclease is an RNA-directed nuclease.

85. The method of claim 84, wherein the RNA-directed nuclease is a Cas protein.

86. The method of claim 85, wherein the Cas protein is codon optimized for expression in the cell and/or is a Type-II Cas protein or a Type-V Cas protein.

87. The method of claim 86, wherein the cell is a eukaryotic cell.

88. The method of claim 87, wherein the eukaryotic cell is a mammalian cell (e.g., a human cell).

89. The method of any one of claims 70-88, wherein the system is packaged into a single vector.

90. The method of claim 89, wherein the single vector is a viral vector or a plasmid.
